(19) United States
(12) Patent Application Publication
(10) Pub. No.: US 2021/0107969 A1
(43) Pub. Date: Apr. 15, 2021

(54) MOLECULES, COMPOSITIONS AND METHODS FOR TREATMENT OF CANCER

(71) Applicants: THE JOHNS HOPKINS UNIVERSITY, Baltimore, MD (US); Y-TRAP, INC., Baltimore, MD (US)

(72) Inventors: Atul Bedi, Timonium, MD (US); Rishi Bedi, San Francisco, CA (US)

(21) Appl. No.: 16/925,068

(22) Filed: Jul. 9, 2020

Related U.S. Application Data
(60) Provisional application No. 62/872,194, filed on Jul. 9, 2019.

(51) Int. Cl. C07K 14/71; C07K 16/28; A61P 35/00; A61K 45/06; A61K 39/395; A61K 38/17; C07K 16/22; C07K 16/32

(52) U.S. Cl. CPC C07K 14/71; C07K 16/2803; A61P 35/00; A61K 45/06; A61K 39/3955; A61K 38/179; A61K 2039/505; C07K 16/22; C07K 16/2827; C07K 16/2818; C07K 16/32; C07K 2319/30; C07K 2317/76; C07K 16/2863

(57) ABSTRACT

The present invention relates generally to the field of multifunctional fusion proteins to counteract immune dysfunction in the tumor microenvironment and more specifically to compositions and methods employing such fusion proteins (either alone or in combination regimens) for treatment of cancer.

### CROSS-REFERENCE TO RELATED APPLICATIONS

This application claims benefit of priority under 35 U.S.C. § 119(e) of U.S. Ser. No. 62/872,194, filed Jul. 9, 2019, the entire contents of which is incorporated herein by reference in its entirety.

### STATEMENT OF GOVERNMENT SUPPORT

This invention was made with government support under CA184199 and DE019032 awarded by the National Institutes of Health. The government has certain rights in the invention.

### INCORPORATION OF SEQUENCE LISTING

The material in the accompanying sequence listing is hereby incorporated by reference into this application. The accompanying sequence listing text file, name JHU4140_1WO_Sequence_Listing, was created on ______, and is ______ kb. The file can be accessed using Microsoft Word on a computer that uses Windows OS.

### BACKGROUND OF THE INVENTION

### Field of the Invention

The present invention relates generally to the field of multifunctional fusion proteins to counteract immune dysfunction in the tumor microenvironment and more specifically to compositions and methods employing such fusion proteins (either alone or in combination regimens) for treatment of cancer.

### Background Information

Genetic mutations accruing from the inherent genomic instability of tumor cells present neo-antigens that are recognized by the immune system. Cross-presentation of tumor antigens at the immune synapse between antigen-presenting dendritic cells and T lymphocytes can potentially activate an adaptive antitumor immune response that is mediated by CD4 T-helper cells (T_{H}1) and CD8⁺ cytotoxic effector cells, and sustained by tumor-reactive central memory T cells¹. However, tumors continuously evolve to counteract and ultimately defeat such immune surveillance by co-opting and amplifying mechanisms of immune tolerance to evade elimination by the immune system. This prerequisite for tumor progression is enabled by the ability of cancers to produce multiple immunomodulatory factors that create a tolerogenic or dysfunctional immune cell microenvironment. The tumor microenvironment is enriched with multiple cytokines and ligands that act in concert to alter the recruitment, differentiation, activation, or effector function of immune cells (T cells, macrophages, dendritic cells, NK cells), thereby resulting in key signatures of immune dysfunction that enable tumorigenesis, tumor progression, and metastases: (1) Immune tolerance: via expression of ligands that suppress the activation or function of immune cells (T cells, NK cells, macrophages, DCs) and skew their differentiation toward an immuno-inhibitory phenotype (e.g. regulatory T cells, Tregs); (2) Tumor promoting inflammation; via expression of ligands that skew the differentiation of immune cells toward a phenotype (e.g. TH17 cells; M2 macrophages) that in turn, express cytokines and ligands which promote multiple tumor cell proliferation/survival, tumor angiogenesis, and metastases. As such the key signatures of immune dysfunction in the tumor microenvironment (TME) (immune tolerance and tumor-promoting inflammation) involve complex multipronged cross-talk between tumor cells and tumor-infiltrating immune cells. The key molecular determinants of such deleterious cross-talk involve interactions between ligands expressed on tumor cells and their cognate receptors on tumor-infiltrating immune cells (T cells, macrophages, DC, NK cells), or conversely, ligands expressed on immune cells (e.g. Tregs, TH17 cells) and their cognate receptors on tumor cells or tumor-associated cells (e.g endothelial cells, CAFs).

Efforts to counteract immune dysfunction in the TME are currently stymied or limited by the following key therapeutic challenges: (1) The plethora of ligands that act independently or in concert to create the dysfunctional TME. As such, therapeutic agents that address a specific molecular determinant fails to counteract other redundant or orthogonal ligands that are concurrently or adaptively upregulated to create the dysfunctional immune signature (immune tolerance or tumor-promoting inflammation); (2) The complexity and promiscuity of ligand-receptor interactions that operate in the TME. Tumor cell-immune cell cross-talk involves multiple autocrine and paracrine ligand-receptor interactions (in cis and trans) that maintain the abnormal phenotype of both tumor cells and tumor-infiltrating immune cells. Many ligands interact with more than one receptor, and in some instances the same ligand can have disparate, opposing, or bidirectional effects when it interacts with different receptors on a T cell; the effect of a ligand-receptor interaction on an immune cell is further influenced by other ligand-receptor signals that may simultaneously operate in the TME; (3) Stifling the molecular determinants of immune tolerance (for e.g. specific immune checkpoints, such as T cell co-inhibitory molecules) may fail to counteract, or even counterproductively exacerbate, ligand-receptor(s) that cause tumor-promoting inflammation and angiogenesis; (4) The selective or preferential localization of therapeutic molecules to the TME is required to effectively disrupt autocrine/paracrine ligand-receptor interactions that are hyperactive in the localized microenvironment of a tumor cell, tumor-infiltrating immune cell, or tumor-infiltrating endothelial cell.

The present invention describes novel multifunctional molecules that are designed to address these therapeutic challenges that limit current cancer therapy. The molecules of the invention are designed to simultaneously counteract one or more of the key determinants of the key signatures of the tumor microenvironment: (1) Immune cell suppression and immune tolerance; (2) tumor-promoting inflammation; (3) elevated neoangiogenesis. These signatures are ubiquitous hallmarks of cancers that are key determinants of tumor progression as well as their resistance to current anticancer therapies. Since immune dysfunction and angiogenesis are also the Achilles' heel of cancers, the multifunctional molecules of the invention may provide effective immunotherapeutic strategies. The invention also describes methods of treatment of cancers that attempt to address these therapeutic challenges. These methods include but are not limited to methods that utilize novel multifunctional molecules of the invention for cancer immunotherapy, either alone or in combination regimens.

### SUMMARY OF THE INVENTION

The present invention is based on the seminal discovery that fusion proteins comprising at least one ligand binding sequence of the extracellular domain of a protein and a targeting moiety are effective at treating various diseases and disorders.

The molecules of the invention are fusion proteins comprising at least one ligand-binding sequence of the extracellular domain (ECD, or “ligand trap”) or fragment thereof of a naturally-occurring protein, or modified version or fragment thereof.

In some embodiments, the fusion proteins of the invention comprise a targeting polypeptide to which one or more ECDs are fused. “X” is a molecule that is specifically bound by the targeting polypeptide. “Y” is a ligand-binding sequence of an extracellular domain, or fragment thereof. In some embodiments, the fusion proteins of the invention have the structure “anti-{X}-{Y}”, where the ligand-binding sequence of the extracellular domain Y is fused to the targeting polypeptide. In some embodiments, the targeting polypeptide is an antibody that comprises at least one heavy chain and one light chain. In some embodiments, Y is fused to the C terminus of the light chain of the antibody. In other embodiments, Y is fused to the C terminus of the heavy chain of the antibody. In some embodiments, Y is fused to the N terminus of the light chain of the antibody. In other embodiments, Y is fused to the N terminus of the heavy chain of the antibody.

In some embodiments, the targeting polypeptide is an antibody or other polypeptide comprising a heavy chain and light chain connected by one or more disulfide bonds. “X” is a molecule that is specifically bound by this targeting polypeptide. “Y” is a ligand-binding sequence of an extracellular domain, or fragment thereof “Z” is a ligand-binding sequence of a different extracellular domain, or fragment thereof.

In some embodiments, the fusion proteins of the invention have the structure “anti-{X}-{Y}-{Z}”, where Y and Z are fused to the polypeptide that binds X. In some embodiments, Y is fused to the C terminus of the heavy chain of the antibody and Z is fused to the C terminus of the light chain of the antibody. In other embodiments, Y is fused to the C terminus of the light chain of the antibody and Z is fused to the C terminus of the heavy chain of the antibody. In some embodiments, Y is fused to the N terminus of the heavy chain of the antibody and Z is fused to the N terminus of the light chain of the antibody. In other embodiments, Y is fused to the N terminus of the light chain of the antibody and Z is fused to the N terminus of the heavy chain of the antibody.

In some embodiments, an ECD of the invention may be modified in one or more of the following ways: (1) substitution or deletion of residues that are not necessary for ligand binding, (2) substitution of residues to remove N-linked glycosylation sites, (3) substitution, addition, or deletion of residues to increase affinity to one or more of its cognate ligands, (4) substitution, addition, or deletion of residues to improve the expression of the fusion protein, (5) substitution, addition, or deletion of residues to allow for site-specific conjugation of drug conjugates, (6) substitution, addition, or deletion of residues to decrease the specificity of the ligand trap to one or more of its cognate ligands while maintaining or increasing its specificity to other cognate ligands, (7) fusion of non-continuous domains of the same ECD, (8) fusion of domains from different isoforms of the same ECD, (9) fusion of domains from different members of the same ECD family. In some embodiments, any of these modifications refer to the same ECD if they result in a sequence that maintains 90%, 95%, 98%, or 99% sequence identity to a ligand-binding sequence of the ECD.

In some embodiments, the fusion proteins comprise two ECDs (ECD #1, ECD #2) fused together. In some embodiments, the fusion protein additionally comprises a Fc domain. In some embodiments, the fusion protein additionally comprises a linker. In some embodiments, the structure of the fusion protein is N (terminus)-ECD #1-ECD #2-C (terminus). In other embodiments, the structure is N-ECD #2-ECD #1-C. In other embodiments, the structure is N-ECD #1-linker-ECD #2-C or N-ECD #2-linker-ECD #1-C. In other embodiments, the structure is N-ECD #1-Fc-ECD #2-C or N-ECD #2-linker-ECD #1-C. In other embodiments, the structure is N-ECD #1-Fc-linker-ECD #2-C, or N-ECD #2-Fc-linker-ECD #1-C.

In one aspect, component parts of the fusion proteins of the invention are fused via a flexible linker. In a further aspect, the flexible linker comprises the polypeptide sequence (GGGGS)n where n is between 1 and 10. In another aspect, the flexible linker is selected from the following list: (GGGGS)3 (SEQ ID NO: 200), (GGGGS)4 (SEQ ID NO: 201), waldo1999 (SEQ ID NO: 202), bird1988-1 (SEQ ID NO: 203), bird1988-2 (SEQ ID NO: 204). In one aspect, a linker may be used to fuse an ECD to a targeting polypeptide. In another aspect, a linker may be used to fuse one ECD to another. In another aspect, a linker may be used to fuse an ECD to the C terminus of the CH3 region of the heavy chain of an Fc polypeptide.

In various embodiments, the fusion proteins of the invention comprise one or more of the following ECDs: (1) a ligand-binding sequence of an extracellular domain of TGFbR (e.g., TGFbRII ECD), or fragment thereof. In one aspect, this ECD binds TGFb1, TGFb2, and/or TGFb3; (2) a ligand-binding sequence of an extracellular domain of PD-1 (e.g., PD1 ECD), or fragment thereof. In one aspect, this ECD binds PD-L1 and/or PD-L2. In one embodiment, this ligand trap has one or more amino acid substitutions which increase its affinity for PD-L1 and/or PD-L2; (3) a ligand-binding sequence of an extracellular domain of VEGFR (e.g., VEGFR1, VEGFR2, VEGFR3), or fragment thereof, or a fusion of VEGF-binding sequences of one or more VEGFR extracellular domains (e.g., VEGFR1 domain 2 fused to VEGFR2 domain 3). In one aspect, this ECD binds VEGFA, VEGFB, VEGFC, and/or PIGF; (4) a ligand-binding sequence of an extracellular domain of TIM-3 (e.g., TIM3 ECD), or fragment thereof, or a hypoglycosylated variant of TIM-3, or fragment thereof. In one aspect, this ECD binds CEACAM1, CEACAM5, phosphatidyl-serine, and/or Galectin-9; (5) a ligand-binding sequence of an extracellular domain of SIRPa (e.g., SIRPa-ECD), or fragment thereof; or a hypoglycosylated variant of SIRPa, or fragment thereof. In one aspect, this ECD binds CD47; (6) a ligand-binding sequence of an extracellular domain of B- and T-lymphocyte attenuator (BTLA ECD) or fragment thereof, or a hypoglycosylated variant of BTLA or fragment thereof. In one aspect, this ECD binds herpesvirus entry mediator (HVEM); (7) a ligand-binding sequence of an extracellular domain of SIGLEC10 or fragment thereof, or a hypoglycosylated variant of SIGLEC10 or fragment thereof. In one aspect, this ECD binds CD24.

In one aspect, the targeting polypeptide (TP) comprises an antigen-binding domain of an immunoglobulin, antibody, bispecific or multispecific antibody, antibody fragment, single chain variable fragment (scFv), bivalent or multivalent scFv, Affimer, a ligand-binding sequence from the extracellular domain (ECD) of a receptor, or Fc-containing polypeptide. In certain aspects, the targeting polypeptide is an antibody.

In some embodiments, the targeting polypeptide is an antibody and this antibody is fused to one or more ECDs. In such cases, the fusion protein comprising an antibody and one or more ECDs may be referred to as an “antibody-ligand trap”, or “ALT”, which are used interchangeably.

In some embodiments, this targeting polypeptide binds a tumor-associated antigen or tumor antigen. In one embodiment, a “tumor-associated antigen” is a molecule whose expression is elevated on tumor cells. In one embodiment, the tumor-associated antigen is a growth factor receptor or a growth factor. In some embodiments, the growth factor or growth factor receptor may be selected from the following list: EGFR, EGFRvIII, HER2, HER3, PDGF, PDGFR, HGF, HGFR, IGF, IGF1R, VEGF, VEGFR, TGFb, TGFbR, FGF, FGFR.

In one embodiment, the fusion protein of the invention comprises a targeting polypeptide that specifically binds a tumor cell surface molecule. In some embodiments, the targeting polypeptide binds one of the following targets: CA125, CA19-9, CD30, CEACAM5, CEACAM1, CEACAM6, DLL3, DLL4, DPEP3, EGFR, EGFRvIII, GD2, HER2, HER3, HGF, IGF1R, IL13Ra2, LIV-1, LRRC15, MUC1, PRLR, PSCA, PSMA, PTK7, SEZ6, SLAMF7, TF, cMet, claudin, mesothelin, nectin4, uPAR, GPNMB, CD79b, CD22, NaPi2b, SLTRK6, STEAP1, MUC16, CD37, GCC, AGC-16, 5T4, CD70, TROP2, CD74, CD27L, Fra, CD138, CA6.

In some embodiments, the targeting polypeptide binds an antigen overexpressed by a hematologic malignancy. In some embodiments, the targeting polypeptide binds an antigen overexpressed by multiple myeloma. In some embodiments, the targeting polypeptide binds CD38, SLAMF7, or BCMA. In some embodiments, the targeting polypeptide is an antibody selected from the following list: MEDI2228; CC-99712; belantamab; Gemtuzumab (anti-CD33 mAb). In some embodiments, the antibody binds CD20. In some embodiments, the targeting polypeptide binds rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb). In some embodiments, the targeting polypeptide binds CD19. In some embodiments, the antibody binds CD30, or CD22. In some embodiments, the targeting polypeptide binds an antigen overexpressed by leukemia. In some embodiments, the targeting polypeptide binds CD33.

In the case that the targeting polypeptide is a bispecific antibody (bsAb), it may be an obligate or non-obligate bsAb. In some embodiments, one of the targets of the bsAb is CD3. In one aspect, the bsAb may be a CrossMab or a BiTE. Examples of bsAbs that may be used as targeting polypeptides of the fusion proteins of the invention include the following: CD3×B7-H3 (e.g., orlotamab), CD3×BCMA (e.g., AMG420, AMG701, EM801, JNJ-64007957, PF-06863135, REGN5458), CD3×CD19 (e.g., A-319, AFM11, AMG562, blinatumomab), CD3×CD20 (e.g., mosunetuzumab, plamatomab, REGN1979, CD20-TCB), CD3×CD33 (e.g., AMG330, AMG673, AMV-564, GEM333), CD3×CD38 (e.g., AMG424, GBR1342), CD3×CEA (e.g., Cibisatamab), CD3×EGFRvIII (e.g., AMG596), CD3×EpCAM (e.g., A-337, catumaxomab, removab), CD3×FLT3 (e.g., AMG427), CD3×GPC3 (e.g., ERY974), CD3×gpA33 (e.g., MGD007), CD3×GPRC5D (e.g., JNJ-64407564), CD3×HER2 (e.g., GBR1302, M802, RG6194), CD3×MUC16 (e.g., REGN4018), CD3×P-Cadherin (e.g., PF-06671008), CD3×PSMA (e.g., AMG160, MOR209, pasotuxizumab), CD3×SSTR2 (e.g., tidutamab), CD40×MSLN (e.g., ABBV-428), PD-1×ICOS (e.g., Xmab23104), or PD-L1×4-1BB (e.g., MCLA-145), or CTLA-4×PD-1.

In one embodiment, the fusion protein of the invention comprises a targeting polypeptide that specifically binds a “don't eat me” ligand or receptor that inhibits the function of macrophages, dendritic cells, or other innate immune cells. “Don't eat me” ligands expressed by cells bind their cognate receptor on a macrophage, dendritic cell, or other innate immune cell to inhibit phagocytosis. Tumor cells take advantage of this anti-phagocytic mechanism and overexpress “don't eat me” ligands in order to inhibit innate immune cell antitumor activity. In some embodiments, the targeting polypeptide binds CD47, SIRPa, CD31, CD24, SIGLEC10, or LILRB1.

In one embodiment, the fusion protein of the invention comprises a targeting polypeptide that specifically binds a T cell inhibitory receptor (TCIR), a T cell inhibitory receptor ligand (TCIR ligand), a T-cell co-inhibitory molecule, or a T cell co-stimulatory molecule.

In an additional aspect, the antibody is an antagonist of a TCIR, TCIR ligand, or T cell co-inhibitory molecule. In an additional aspect, the targeting moiety polypeptide specifically binds one or more of the following molecules: Cytotoxic T lymphocyte associated antigen-4 (CTLA-4, CD152), Programmed Death-1 protein (PD-1), Programmed death ligand-1 (PD-L1), Programmed death ligand (PD-L2), B7-H3 (CD276), T-cell immunoglobulin and mucin-domain containing-3 (TIM-3), Carcinoembryonic antigen-related cell adhesion molecule (CEACAM), V domain Ig suppressor of T cell activation (VISTA), V-set and immunoglobulin domain containing 8 (VSIG8), B and T lymphocyte attenuator (BTLA), Herpesvirus Entry Mediator (HVEM), CD160, T cell Ig and ITIM domain (TIGIT), PVRIG, CD226, CD96, Lymphocyte activation gene-3 (LAG-3).

In another aspect, the targeting polypeptide is an agonist of a T cell co-stimulatory molecule. In one aspect, the targeting polypeptide is an antibody that binds a T cell co-stimulatory molecule as an agonist. In another aspect, the targeting polypeptide is the extracellular domain of a native agonist ligand of a T cell co-stimulatory molecule. In an additional aspect, the targeting polypeptide specifically binds one of the following molecules: 4-1BB (CD137), Inducible T-Cell Costimulator (ICOS), OX-40 (CD134), Herpesvirus Entry Mediator (HVEM), glucocorticoid-induced TNFR-related protein (GITR), CD40, CD30, DNAM, or CD27.

In one embodiment, the fusion protein of the invention comprises a targeting polypeptide that specifically binds a cytokine or cytokine receptor. In a preferred embodiment, the cytokine/cytokine receptor interaction contributes to immune tolerance and/or promotion of tumor-promoting inflammation. In some embodiments, the cytokine or cytokine receptor are selected from the following: IL-17, IL-17R, IL-23, IL-23R, IL-6, IL-6R, IL-1, IL-1R, IL-10, IL-10R, TGFb, or TGFbR.

In one embodiment, the fusion protein of the invention comprises a targeting polypeptide that specifically binds a ectonucleotidase. In some embodiments, the ectonucleotidase is CD39 or CD73.

In one embodiment, the invention comprises fusion proteins comprising targeting polypeptides wherein the targeting polypeptide is an antibody fused to one or more ECDs. In one aspect, the targeting polypeptide is an antibody-drug conjugate (ADC). In one aspect, the antibody is conjugated to one or more cytotoxic agents. In some embodiments, the cytotoxic agent causes immunogenic cell death. In some embodiments, the cytotoxic agent causes genotoxic cell death.

The cytotoxic agent conjugated to the targeting polypeptide antibody may be any agent that induces cell death. In various embodiments, the cytotoxic agent may be selected from, but is not limited to, the following list: (1) maytansinoid (DM1), (2) calcheamicin, (3) auristatin (e.g., monomethyl auristatin E (MMAE) or monomethyl auristatin F (MMAF)).

In some embodiments, the cytotoxic agent may be conjugated to cysteines. In other embodiments, the cytotoxic agent may be conjugated to lysines. In some embodiments, the cytotoxic agent may be conjugated via a cleavable linker. In some embodiments, the cytotoxic agent may be conjugated via a non-cleavable linker. In various embodiments, the cytotoxic agent may be linked to the targeting polypeptide antibody via a linker, which may be selected from, but is not limited to, the following list: (1) hydrazone, (2) SMCC (maleimide), (3) valine-citrulline, (4) 4AP, (5) maleimidocaproyl (mc), (6) maleimidomethyl cyclohexane-1-carboxylate (mcc). The linker may further comprise one or more spacers. In some embodiments, the spacer may be selected from thiol-reactive maleimidocaproyl spacer and p-amino-benzyloxycarbonyl spacer. In one embodiment, the cleavable linker is maleimidocaproyl-valyl-citrullinyl-p-aminobenzyloxycarbonyl (mc-val-cit-PABC).

In one embodiment, a tumor-targeted antibody is fused to one or more receptor extracellular domains and conjugated to one or more cytotoxic agents. In one embodiment, a fusion protein comprises a targeting polypeptide wherein the targeting polypeptide is an antibody-drug conjugate and the targeting polypeptide is fused to a ligand-binding sequence of a receptor ECD. In one aspect, the receptor ECD is fused to the heavy chain of the targeting polypeptide. In another aspect, the receptor ECD is fused to the light chain of the targeting polypeptide.

In one embodiment, a fusion protein comprises a targeting polypeptide wherein the targeting polypeptide is an antibody-drug conjugate and the targeting polypeptide is fused to a ligand-binding sequence of TGFbRII ECD, or a fragment thereof. In one embodiment, a fusion protein comprises a targeting polypeptide wherein the targeting polypeptide is an antibody-drug conjugate and the targeting polypeptide is fused to a ligand-binding sequence of PD1 ECD, or a fragment thereof. In one embodiment, a fusion protein comprises a targeting polypeptide wherein the targeting polypeptide is an antibody-drug conjugate and the targeting polypeptide is fused to a ligand-binding sequence of BTLA ECD, or a fragment thereof. In one embodiment, a fusion protein comprises a targeting polypeptide wherein the targeting polypeptide is an antibody-drug conjugate and the targeting polypeptide is fused to a ligand-binding sequence of TIM-3 ECD, or a fragment thereof. In one embodiment, a fusion protein comprises a targeting polypeptide wherein the targeting polypeptide is an antibody-drug conjugate and the targeting polypeptide is fused to a ligand-binding sequence of SIRPa ECD, or a fragment thereof. In one embodiment, a fusion protein comprises a targeting polypeptide wherein the targeting polypeptide is an antibody-drug conjugate and the targeting polypeptide is fused to a ligand-binding sequence of SIGLEC10 ECD, or a fragment thereof. In one embodiment, a fusion protein comprises a targeting polypeptide wherein the targeting polypeptide is an antibody-drug conjugate and the targeting polypeptide is fused to a ligand-binding sequence of VEGFR ECD, or a fragment thereof.

In various embodiments, the targeting polypeptide is an antibody-drug conjugate selected from: gemtuzumab ozogamicin, brentuximab vedotin, trastuzumab emtansine, inotuzumab ozogamicin, polatuzumab vedotin, enfortumab vedotin, trastuzumab deruxtecan, or sacituzumab govitecan.

In one embodiment, the fusion protein comprises anti-nectin-4 antibody fused to TGFbRII on the C-terminus of the heavy chain; and MMAE is conjugated to the antibody via a protease cleavable linker comprising maleimidocaproyl, valine-citrulline, and PABC. In one aspect, this fusion protein is anti-nectin4-TGFbRII (e.g., SEQ ID NOs: 265, 160). In one embodiment, the fusion protein comprises anti-nectin-4 antibody fused to BTLA on the C-terminus of the heavy chain; and MMAE is conjugated to the antibody via a protease cleavable linker comprising maleimidocaproyl, valine-citrulline, and PABC. In one aspect, this fusion protein is anti-nectin4-BTLA (e.g., SEQ ID NOs: 256, 160). In one embodiment, the fusion protein comprises anti-nectin-4 antibody fused to SIRPa on the C-terminus of the heavy chain; and MMAE is conjugated to the antibody via a protease cleavable linker comprising maleimidocaproyl, valine-citrulline, and PABC. In one aspect, this fusion protein is anti-nectin4-SIRPa (e.g., SEQ ID NOs: 264, 160). In one embodiment, the fusion protein comprises anti-nectin-4 antibody fused to PD1 on the C-terminus of the heavy chain; and MMAE is conjugated to the antibody via a protease cleavable linker comprising maleimidocaproyl, valine-citrulline, and PABC. In one aspect, this fusion protein is anti-nectin4-PD1 (e.g., SEQ ID NOs: 261, 160). In one embodiment, the fusion protein comprises anti-nectin-4 antibody fused to TIM3 on the C-terminus of the heavy chain; and MMAE is conjugated to the antibody via a protease cleavable linker comprising maleimidocaproyl, valine-citrulline, and PABC. In one aspect, this fusion protein is anti-nectin4-TIM3 (e.g., SEQ ID NOs: 266, 160). In one embodiment, the fusion protein comprises anti-nectin-4 antibody fused to SIGLEC10 on the C-terminus of the heavy chain; and MMAE is conjugated to the antibody via a protease cleavable linker comprising maleimidocaproyl, valine-citrulline, and PABC. In one aspect, this fusion protein is anti-nectin4-SIGLEC10 (e.g., SEQ ID NOs: 263, 160).

In one embodiment, the fusion protein comprises anti-HER2 antibody fused to TGFbRII on the C-terminus of the heavy chain; and DM1 is conjugated to the antibody via a linker comprising mcc. In one aspect, this fusion protein is anti-HER2-TGFbRII (e.g., SEQ ID NOs: 253, 55). In one embodiment, the fusion protein comprises anti-HER2 antibody fused to BTLA on the C-terminus of the heavy chain; and DM1 is conjugated to the antibody via a linker comprising mcc. In one aspect, this fusion protein is anti-HER2-BTLA (e.g., SEQ ID NOs: 244, 55). In one embodiment, the fusion protein comprises anti-HER2 antibody fused to TIM-3 on the C-terminus of the heavy chain; and DM1 is conjugated to the antibody via a linker comprising mcc. In one aspect, this fusion protein is anti-HER2-TIM3 (e.g., SEQ ID NOs: 254, 55). In one embodiment, the fusion protein comprises anti-HER2 antibody fused to PD1 on the C-terminus of the heavy chain; and DM1 is conjugated to the antibody via a linker comprising mcc. In one aspect, this fusion protein is anti-HER2-PD1 (e.g., SEQ ID NOs: 249, 55). In one embodiment, the fusion protein comprises anti-HER2 antibody fused to SIRPa on the C-terminus of the heavy chain; and DM1 is conjugated to the antibody via a linker comprising mcc. In one aspect, this fusion protein is anti-HER2-SIRPa (e.g., SEQ ID NOs: 252, 55). In one embodiment, the fusion protein comprises anti-HER2 antibody fused to SIGLEC10 on the C-terminus of the heavy chain; and DM1 is conjugated to the antibody via a linker comprising mcc. In one aspect, this fusion protein is anti-HER2-SIGLEC10 (e.g., SEQ ID NOs: 251, 55). In one embodiment, the fusion protein comprises anti-HER2 antibody fused to VEGFR on the C-terminus of the heavy chain; and DM1 is conjugated to the antibody via a linker comprising mcc. In one aspect, this fusion protein is anti-HER2-VEGFR (e.g., SEQ ID NOs: 255, 55).

The targeting polypeptide may be an Fc-containing polypeptide, and the CH3 region of the Fc may end with a terminal lysine. In some embodiments, the terminal lysine of the CH3 region of the Fc may be removed.

The fusion proteins of the invention are designed to counteract the molecular determinants that contribute to key signatures of the tumor microenvironment. The targeting polypeptide and/or ECDs counteract one or more of the key receptor/ligand interactions that underlie the following signatures in the TME.

The first signature of the tumor microenvironment is immune tolerance, characterized by the following: (a) suppression of the differentiation, maturation, and function of macrophages/dendritic cells mediated by “don't-eat-me” signals (e.g., CD47/SIRPa, CD31/CD31, SIGLEC10/CD24, LILRB1/MHC), immuno-inhibitory cytokines (e.g., TGFb/TGFbR), immuno-inhibitory molecules that signal via SHP1/SHP2 (e.g., PD1/PDL1/PDL2); (b) inhibition of tumor-relative T cell maturation, activation, and function mediated by T cell co-inhibitory molecules (e.g., PD1/PDL1/PDL2, CTLA-4, LAG3, BTLA/HVEM, TIGIT/PVRIG, TIM3/CEACAM, VISTA/VSIG8), and immunosuppressive molecules involved in Treg differentiation and/or function (e.g., CTLA-4, TGFb, CD39, CD73, IL-10, HVEM).

The second signature of the tumor microenvironment is tumor promoting inflammation, characterized by the following (a) induction and maintenance of TH17 cells in the TME mediated by cytokine/cytokine receptor interactions (e.g., IL-6/IL-6R, IL-23/IL-23R, TGFb/TGFbR, IL-1/IL-1R), (b) TH17 function & TH17/tumor cell/endothelial cell crosstalk mediated by cytokine/cytokine receptor interactions (e.g., TGFb/TGFbR, IL-17/IL-17R, VEGF/VEGFR), (c) promotion of neoangiogenesis mediated by cytokine/cytokine receptor interactions (e.g., VEGF/VEGFR, TGFb/TGFbR, IL-17/IL-17).

In some embodiments, the fusion proteins of the invention are preferentially localized to a component of the tumor microenvironment. In one aspect, the fusion protein comprises a targeting polypeptide and this targeting polypeptide binds a component of the tumor microenvironment to localize the fusion protein. In a further aspect, the fusion protein comprises a targeting polypeptide and this targeting polypeptide binds to a tumor cell surface molecule, or tumor-infiltrating immune cell surface molecule, thereby localizing the fusion protein to the immediate microenvironment of the targeted tumor cell, tumor-associated endothelial cell, or tumor-infiltrating T cell (e.g., Treg or TH17). In another aspect, an ECD of the fusion protein binds a component of the tumor microenvironment to localize the fusion protein. In a further aspect, an ECD of the fusion protein binds to a tumor cell surface molecule, or tumor-infiltrating immune cell surface molecule, thereby localizing the fusion protein to the immediate microenvironment of the targeted tumor cell, tumor-associated endothelial cell, or tumor-infiltrating T cell (e.g., Treg or TH17).

In addition to localizing the fusion protein, in some embodiments, the targeting polypeptide additionally exerts a function by neutralizing a receptor/ligand interaction that aggravates immune tolerance or tumor promoting inflammation. In other embodiments, the targeting polypeptide exerts a function by neutralizing a growth factor, growth factor receptor, or other molecule that promotes tumor cell survival, growth, or metastases. In other embodiments, the targeting polypeptide serves as an agonist that binds a T cell co-stimulatory molecule.

In various embodiments, the fusion proteins of the invention counteract VEGF in the tumor microenvironment.

In some embodiments, the fusion proteins of the invention comprise a targeting polypeptide that binds VEGF or VEGFR fused to one or more receptor ECDs. These receptor ECDs are preferably selected from the following: PD1 ECD, TIM-3 ECD, TGFbRII ECD, BTLA ECD, SIRPa ECD, SIGLEC10 ECD. In some embodiments, the targeting polypeptide that binds VEGF or VEGFR is an antibody. Exemplary embodiments include anti-VEGF-TGFbR (e.g., SEQ ID NOs: 370, 32); anti-VEGF-BTLA (e.g., SEQ ID NOs: 361, 32); anti-VEGF-SIGLEC10 (e.g., SEQ ID NOs: 368, 32); anti-VEGF-PD1 (e.g., SEQ ID NOs: 366, 32); anti-VEGF-SIRPa (e.g., SEQ ID NOs: 369, 32); anti-VEGF-TIM3 (e.g., SEQ ID NOs: 371, 32). In another embodiment, the fusion protein comprises antibody that binds VEGF or VEGFR fused to one receptor ECD on heavy chain and another receptor ECD fused on light chain. Exemplary embodiments include anti-VEGF-SIRPa-SIGLEC10 (e.g., SEQ ID NOs: 369, 363); anti-VEGF-TIM3-BTLA (e.g., SEQ ID NOs: 371, 367); anti-VEGF-BTLA-SIGLEC10 (e.g., SEQ ID NOs: 361, 363); anti-VEGF-TGFbR-SIRPa (e.g., SEQ ID NOs: 370, 364); anti-VEGF-PD1-SIRPa (e.g., SEQ ID NOs: 366, 364); anti-VEGF-BTLA-TIM3 (e.g., SEQ ID NOs: 361, 365); anti-VEGF-TIM3-SIRPa (e.g., SEQ ID NOs: 371, 364); anti-VEGF-SIRPa-BTLA (e.g., SEQ ID NOs: 369, 367); anti-VEGF-SIRPa-TIM3 (e.g., SEQ ID NOs: 369, 365); anti-VEGF-TGFbR-SIGLEC10 (e.g., SEQ ID NOs: 370, 363); anti-VEGF-TGFbR-PD1 (e.g., SEQ ID NOs: 370, 362); anti-VEGF-PD1-SIGLEC10 (e.g., SEQ ID NOs: 366, 363); anti-VEGF-PD1-BTLA (e.g., SEQ ID NOs: 366, 367); anti-VEGF-TGFbR-TIM3 (e.g., SEQ ID NOs: 370, 365); anti-VEGF-SIGLEC10-BTLA (e.g., SEQ ID NOs: 368, 367); anti-VEGF-SIRPa-PD1 (e.g., SEQ ID NOs: 369, 362); anti-VEGF-BTLA-SIRPa (e.g., SEQ ID NOs: 361, 364); anti-VEGF-TGFbR-BTLA (e.g., SEQ ID NOs: 370, 367); anti-VEGF-TIM3-SIGLEC10 (e.g., SEQ ID NOs: 371, 363); anti-VEGF-SIGLEC10-TIM3 (e.g., SEQ ID NOs: 368, 365); anti-VEGF-PD1-TIM3 (e.g., SEQ ID NOs: 366, 365); anti-VEGF-TIM3-PD1 (e.g., SEQ ID NOs: 371, 362); anti-VEGF-SIGLEC10-SIRPa (e.g., SEQ ID NOs: 368, 364); anti-VEGF-BTLA-PD1 (e.g., SEQ ID NOs: 361, 362); anti-VEGF-SIGLEC10-PD1 (e.g., SEQ ID NOs: 368, 362).

In other embodiments, the fusion proteins comprise a ligand-binding sequence of an extracellular domain of VEGFR (e.g., VEGFR ECD). In one aspect, the VEGFR ECD comprises Ig domain 2 from VEGFR1, fused to Ig domain 3 from VEGFR2. In one embodiment, the fusion protein comprises amino acids 103-204 of VEGFR1 fused to amino acids 206-308 of VEGFR2. In one embodiment, the fusion protein comprises the same domains of VEGFR1 and VEGFR2 as aflibercept. In some embodiments, the VEGFR ECD may be selected from the following list: SEQ ID NOS: 184; 185; 186. In a preferred embodiment, if the targeting polypeptide is an antibody, the VEGFR ECD is fused to the C terminus of the heavy chain of the antibody.

In one aspect, the fusion proteins of the invention comprise an antibody that targets a tumor antigen or tumor-associated antigen expressed in the TME, wherein said antibody is fused to a VEGF-binding sequence from one or more extracellular domains of VEGFR (e.g. VEGFR1ECD and/or VEGFR2ECD). Exemplary embodiments include anti-HER2-VEGFR (e.g., SEQ ID NOs: 255, 55); anti-EGFRvIII-VEGFR (e.g., SEQ ID NOs: 243, 47); anti-EGFR-VEGFR (e.g., SEQ ID NOs: 231, 43); anti-nectin4-VEGFR (e.g., SEQ ID NOs: 267, 160).

In one aspect, the fusion proteins of the invention comprise an antibody with VEGFR fused to the heavy chain of the antibody and another receptor ECD fused to the light chain of the antibody. This additional receptor ECD may be selected from BTLA, PD1, SIGLEC10, SIRPa, TIM3. Exemplary embodiments include anti-HER2-VEGFR-PD1 (e.g., SEQ ID NOs: 255, 245); anti-HER2-VEGFR-SIRPa (e.g., SEQ ID NOs: 255, 247); anti-HER2-VEGFR-BTLA (e.g., SEQ ID NOs: 255, 250); anti-HER2-VEGFR-SIGLEC10 (e.g., SEQ ID NOs: 255, 246); anti-HER2-VEGFR-TIM3 (e.g., SEQ ID NOs: 255, 248); and anti-EGFRvIII-VEGFR-BTLA (e.g., SEQ ID NOs: 243, 238); anti-EGFRvIII-VEGFR-TIM3 (e.g., SEQ ID NOs: 243, 236); anti-EGFRvIII-VEGFR-SIGLEC10 (e.g., SEQ ID NOs: 243, 234); anti-EGFRvIII-VEGFR-PD1 (e.g., SEQ ID NOs: 243, 233); anti-EGFRvIII-VEGFR-SIRPa (e.g., SEQ ID NOs: 243, 235).

Antitumor efficacy of CD47 blockade may be limited by disruption of TSP-1/CD47-dependent inhibition of VEGF and angiogenesis. In one embodiment, the fusion protein is a polypeptide comprising an antibody that targets CD47, wherein said antibody is fused to a VEGFR ECD. In one embodiment, this fusion protein is anti-CD47-VEGFR (e.g., SEQ ID NOs: 392, 22). In another aspect, the fusion protein comprises a VEGF-binding sequence from VEGFR ECD and a CD47-binding sequence from one or more extracellular domains of SIRPa (SIRPa ECD). In one embodiment, this fusion protein comprises SIRPa ECD and VEGFR ECD. In one embodiment, this fusion protein is SIRPa-Fc-VEGFR (e.g., SEQ ID NO: 552) or VEGFR-Fc-SIRPa (e.g., SEQ ID NO: 568).

In a further aspect, the fusion protein comprises VEGFR ECD, a polypeptide that inhibits the interaction between CD47 and SIRPa, and another receptor ECD. In some embodiments, this fusion protein comprises anti-CD47 mAb with VEGFR ECD fused to the heavy chain; and the other ECD fused to the light chain. Exemplary embodiments are anti-CD47-VEGFR-TIM3 (e.g., SEQ ID NOs: 392, 386); anti-CD47-VEGFR-SIGLEC10 (e.g., SEQ ID NOs: 392, 385); anti-CD47-VEGFR-BTLA (e.g., SEQ ID NOs: 392, 388); anti-CD47-VEGFR-PD1 (e.g., SEQ ID NOs: 392, 384).

In a further embodiment, the fusion protein is a tumor-targeted antibody-drug conjugate fused to VEGFR ECD. For example, the fusion protein may comprise enfortumab vedotin fused to VEGFR ECD. In a further embodiment, the fusion protein is a bispecific antibody that simultaneously binds a tumor cell and a T cell, fused to VEGFR ECD. For example, the fusion protein may comprise CD3×EGFRvIII (e.g., AMG596) fused to VEGFR ECD or CD3×CEA (e.g., cibisatamab) fused to VEGFR ECD or CD3×HER2 (e.g., GBR1302, M802, RG6194) fused to VEGFR ECD.

In some embodiments, the fusion protein comprises VEGFR ECD and a polypeptide that inhibits TGFb/TGFbR signaling. In some embodiments, the fusion protein comprises VEGFR ECD and anti-TGFb mAb, anti-TGFbR mAb, anti-LAP mAb, or anti-GARP mAb. In a further embodiment, the fusion protein may additionally comprise a receptor ECD fused to the light chain of the antibody. In some embodiments, the fusion protein is selected from anti-TGFb-VEGFR-SIRPa (e.g., SEQ ID NOs: 403, 396); anti-TGFb-VEGFR-SIGLEC10 (e.g., SEQ ID NOs: 403, 395); anti-TGFb-VEGFR-BTLA (e.g., SEQ ID NOs: 403, 399); anti-TGFb-VEGFR-TIM3 (e.g., SEQ ID NOs: 403, 397); anti-TGFb-VEGFR-PD1 (e.g., SEQ ID NOs: 403, 394).

TH17 cells produce IL-17 which is a key determinant of resistance to VEGF blockade. Additionally, endothelial cells on which VEGF act express IL-17R that responds to TH17-produced IL-17. In some embodiments, the fusion protein comprises VEGFR ECD and an antibody that binds and disables IL-17/IL-17R, IL-23/IL-23R, or IL-6/IL-6R. In some embodiments, the fusion protein is selected from the following: anti-IL17-VEGFR, anti-IL17R-VEGFR (e.g., SEQ ID NOs: 336, 63), anti-IL23-VEGFR (e.g., SEQ ID NOs: 348, 75), anti-IL23R-VEGFR. In a further embodiment, the fusion protein comprises an additional receptor ECD selected from the following list: SIGLEC10 ECD, SIRPa ECD, BTLA ECD, PD1 ECD, TIM3 ECD.

In some embodiments, the fusion protein comprises VEGFR ECD and a targeting polypeptide that binds and disables a T cell co-inhibitory molecule. In some embodiments, the targeting polypeptide is an antibody. Exemplary embodiments include BTLA-Fc-VEGFR (e.g., SEQ ID NO: 534), PD1-Fc-VEGFR (e.g., SEQ ID NO: 540), TIM3-Fc-VEGFR (e.g., SEQ ID NO: 564), and anti-PDL1-VEGFR (e.g., SEQ ID NOs: 468, 109).

In some embodiments, the fusion protein comprises VEGFR ECD and an antibody that binds an ectonucleotidase. The ectonucleotidase is preferably CD39 or CD73. In a further embodiment, the fusion protein comprises an additional receptor ECD fused to the light chain of the antibody. Exemplary embodiments include anti-CD39-VEGFR-BTLA and anti-CD73-VEGFR-BTLA (e.g., SEQ ID NOs: 427, 422).

In one embodiment, the fusion protein comprises a polypeptide that binds a T cell co-stimulatory molecule and VEGFR ECD. In some embodiments, the fusion protein is a native T cell co-stimulatory molecule ECD fused to VEGFR (either N-costimulatory ECD-Fc-VEGFR ECD-C, or N-VEGFR ECD-Fc-costimulatory ECD-C). In some embodiments, this fusion protein is selected from: 41BBL-Fc-VEGFR (e.g., SEQ ID NO: 632); OX40L-Fc-VEGFR (e.g., SEQ ID NO: 646); ICOSL-Fc-VEGFR (e.g., SEQ ID NO: 642), VEGFR-Fc-41BBL (e.g., SEQ ID NO: 631); VEGFR-Fc-ICOSL (e.g., SEQ ID NO: 641); VEGFR-Fc-OX40L (e.g., SEQ ID NO: 645). In other embodiments, the fusion protein comprises an antibody or other polypeptide that binds a T cell co-stimulatory molecule fused to VEGFR. This antibody or polypeptide is preferably an agonist of the T cell co-stimulatory molecule. In some embodiments, this fusion protein is selected from: anti-OX40-VEGFR (e.g., SEQ ID NOs: 516, 97); anti-41BB-VEGFR (e.g., SEQ ID NOs: 504, 2); anti-ICOS-VEGFR (e.g., SEQ ID NOs: 528, 59).

In various embodiments, the fusion proteins of the invention counteract TGFb in the tumor microenvironment.

In some embodiments, the fusion proteins of the invention comprise a targeting polypeptide that binds TGFb, TGFbR, LAP, or GARP fused to one or more receptor ECDs. These receptor ECDs are preferably selected from the following: PD1 ECD, TIM-3 ECD, VEGFR ECD, BTLA ECD, SIRPa ECD, SIGLEC10 ECD. In some embodiments, the targeting polypeptide that binds TGFb, TGFbR, LAP, or GARP is an antibody. Exemplary embodiments include anti-TGFb-PD1 (e.g., SEQ ID NOs: 398, 133); anti-TGFb-SIRPa (e.g., SEQ ID NOs: 401, 133); anti-TGFb-TIM3 (e.g., SEQ ID NOs: 402, 133); anti-TGFb-SIGLEC10 (e.g., SEQ ID NOs: 400, 133); anti-TGFb-BTLA (e.g., SEQ ID NOs: 393, 133); anti-TGFb-VEGFR (e.g., SEQ ID NOs: 403, 133). In another embodiment, the fusion protein comprises antibody that binds TGFb, TGFbR, LAP, or GARP fused to one receptor ECD on heavy chain and another receptor ECD fused on light chain. Exemplary embodiments include anti-TGFb-SIRPa-BTLA (e.g., SEQ ID NOs: 401, 399); anti-TGFb-BTLA-TIM3 (e.g., SEQ ID NOs: 393, 397); anti-TGFb-PD1-BTLA (e.g., SEQ ID NOs: 398, 399); anti-TGFb-TIM3-SIGLEC10 (e.g., SEQ ID NOs: 402, 395); anti-TGFb-TIM3-BTLA (e.g., SEQ ID NOs: 402, 399); anti-TGFb-BTLA-SIGLEC10 (e.g., SEQ ID NOs: 393, 395); anti-TGFb-SIGLEC10-TIM3 (e.g., SEQ ID NOs: 400, 397); anti-TGFb-BTLA-PD1 (e.g., SEQ ID NOs: 393, 394); anti-TGFb-SIGLEC10-SIRPa (e.g., SEQ ID NOs: 400, 396); anti-TGFb-VEGFR-BTLA (e.g., SEQ ID NOs: 403, 399); anti-TGFb-PD1-TIM3 (e.g., SEQ ID NOs: 398, 397); anti-TGFb-BTLA-SIRPa (e.g., SEQ ID NOs: 393, 396); anti-TGFb-PD1-SIRPa (e.g., SEQ ID NOs: 398, 396); anti-TGFb-SIGLEC10-PD1 (e.g., SEQ ID NOs: 400, 394); anti-TGFb-SIRPa-TIM3 (e.g., SEQ ID NOs: 401, 397); anti-TGFb-SIRPa-PD1 (e.g., SEQ ID NOs: 401, 394); anti-TGFb-TIM3-PD1 (e.g., SEQ ID NOs: 402, 394); anti-TGFb-VEGFR-TIM3 (e.g., SEQ ID NOs: 403, 397); anti-TGFb-VEGFR-PD1 (e.g., SEQ ID NOs: 403, 394); anti-TGFb-TIM3-SIRPa (e.g., SEQ ID NOs: 402, 396); anti-TGFb-SIRPa-SIGLEC10 (e.g., SEQ ID NOs: 401, 395); anti-TGFb-VEGFR-SIGLEC10 (e.g., SEQ ID NOs: 403, 395); anti-TGFb-PD1-SIGLEC10 (e.g., SEQ ID NOs: 398, 395); anti-TGFb-VEGFR-SIRPa (e.g., SEQ ID NOs: 403, 396); anti-TGFb-SIGLEC10-BTLA (e.g., SEQ ID NOs: 400, 399).

In various embodiments, the fusion proteins of the invention counteract TGFb in the tumor microenvironment. In some embodiments, the fusion proteins comprise a ligand-binding sequence of an extracellular domain of TGFbR (e.g., TGFbRII ECD). In one embodiment, this ligand trap binds TGFb1, TGFb2, and/or TGFb3. In some embodiments, the TGFbR ECD may be a ligand-binding sequence of TGFbRII ECD. In some embodiments, the TGFbR ECD may be a fusion of domains from TGFbRII and TGFbRIII. In some embodiments, the TGFbR ECD may be selected from the following list: SEQ ID NOS: 177; 178; 179; 180

TGFb is known to interfere with phagocytosis and FcR-mediated cross-presentation. In some embodiments, the fusion protein comprises TGFbRII ECD and a polypeptide that inhibits the interaction between CD47 and SIRPa. In one embodiment, this fusion protein is anti-CD47 mAb fused to TGFbRII ECD (anti-CD47-TGFbRII (e.g., SEQ ID NOs: 390, 22)). In another embodiment, this fusion protein comprises SIRPa ECD and TGFbRII ECD. In one embodiment, this fusion protein is SIRPa-Fc-TGFbRII (e.g., SEQ ID NO: 550) or TGFbRII-Fc-SIRPa (e.g., SEQ ID NO: 556).

In other embodiments, the fusion protein comprises TGFbRII ECD, a polypeptide that inhibits the interaction between CD47 and SIRPa, and an additional receptor ECD selected from PD1 ECD, BTLA ECD, TIM-3 ECD, SIGLEC 10 ECD. Exemplary embodiments of this fusion protein include anti-CD47-TGFbRII-PD1 (e.g., SEQ ID NOs: 390, 384), anti-CD47-TGFbRII-BTLA (e.g., SEQ ID NOs: 390, 388) and anti-CD47-TGFbRII-TIM3 (e.g., SEQ ID NOs: 390, 386).

TGFb directly interferes with antibody-dependent cellular cytotoxicity (ADCC) mediated by tumor-targeted antibodies, and cross-presentation. In some embodiments, the fusion protein comprises a tumor-targeted antibody and TGFbRII ECD. In some embodiments, this tumor targeted-antibody binds a tumor growth factor or growth factor receptor. In other embodiments, this tumor targeted antibody binds a tumor cell surface molecule. Exemplary embodiments of this fusion protein include, for example, anti-EGFRvIII-TGFbRII (e.g., SEQ ID NOs: 241, 47), anti-uPAR-TGFbRII (e.g., SEQ ID NOs: 272, 162), anti-PSMA-TGFbRII (e.g., SEQ ID NOs: 279, 121), anti-nectin4-TGFbRII (e.g., SEQ ID NOs: 265, 160).

In other embodiments, the fusion protein comprises TGFbRII ECD, SIRPa ECD, and an antibody with a heavy chain and light chain. In one embodiment, the TGFbRII ECD is fused to the heavy chain of the antibody and the SIRPa ECD is fused to the light chain of the antibody. In other embodiments, the antibody of said fusion protein is a tumor-targeted antibody. In some embodiments, this tumor targeted-antibody binds a tumor growth factor or growth factor receptor. In other embodiments, this tumor targeted antibody binds a tumor cell surface molecule. Exemplary embodiments of this fusion protein include anti-EGFR-TGFbRII-SIRPa (e.g., SEQ ID NOs: 229, 223), anti-HER2-TGFbRII-SIRPa (e.g., SEQ ID NOs: 253, 247), anti-EGFRvIII-TGFbRII-SIRPa (e.g., SEQ ID NOs: 241, 235), anti-uPAR-TGFbRII-SIRPa, and anti-PSMA-TGFbRII-SIRPa. In a further embodiment, the fusion protein comprises a tumor-targeted antibody, TGFbRII fused to the heavy chain, and SIGLEC10 ECD fused to the light chain.

In a further embodiment, the fusion protein is a tumor-targeted antibody-drug conjugate fused to TGFbRII ECD. For example, the fusion protein may comprise enfortumab vedotin fused to TGFbRII ECD. In a further embodiment, the fusion protein is a bispecific antibody that simultaneously binds a tumor cell and a T cell, fused to TGFbRII ECD. For example, the fusion protein may comprise CD3×EGFRvIII (e.g., AMG596) fused to TGFbRII ECD or CD3×CEA (e.g., cibisatamab) fused to TGFbRII ECD or CD3×HER2 (e.g., GBR1302, M802, RG6194) fused to TGFbRII ECD.

In a further embodiment, the fusion protein comprises a tumor-targeted antibody, TGFbRII fused to the heavy chain, and an additional receptor ECD fused to the light chain of the antibody selected from one of the following: PD1 ECD, BTLA ECD, TIM-3 ECD. Exemplary embodiments of this fusion protein include anti-PSMA-TGFbRII-PD1, anti-PSMA-TGFbRII-BTLA, anti-nectin4-TGFbRII-PD1 (e.g., SEQ ID NOs: 265, 257), or anti-nectin4-TGFbRII-BTLA (e.g., SEQ ID NOs: 265, 262).

In some embodiments, the fusion protein comprises TGFbRII ECD and an antibody that binds and disables a T cell co-inhibitory molecule. Exemplary embodiments of the invention are anti-PVRIG-TGFbRII and anti-TIGIT-TGFbRII (e.g., SEQ ID NOs: 478, 139). In some embodiments, the fusion protein comprises TGFbRII ECD, an antibody that binds and disables a T cell co-inhibitory molecule, and an additional receptor ECD. In one aspect, the TGFbRII ECD is fused to the heavy chain of the antibody and the additional receptor ECD is fused to the light chain of the antibody. The T cell co-inhibitory molecule may be selected from the following: PD1, PDL1, CTLA4, TIGIT, TIM3. In other embodiments, the fusion protein comprises TGFbRII ECD and the ECD of a T cell co-inhibitory molecule, selected from BTLA ECD, TIM-3 ECD. Exemplary embodiments of the invention are anti-PDL1-TGFbRII-BTLA (e.g., SEQ ID NOs: 466, 464), anti-CTLA4-TGFbRII-BTLA (e.g., SEQ ID NOs: 444, 441), anti-PD1-TGFbRII-BTLA (e.g., SEQ ID NOs: 456, 453), and anti-TIGIT-TGFbRII-BTLA (e.g., SEQ ID NOs: 478, 475).

In some embodiments, the fusion protein comprises TGFbRII ECD and an antibody that binds an ectonucleotidase. The ectonucleotidase is preferably CD39 or CD73. In a further embodiment, the fusion protein comprises an additional receptor ECD fused to the light chain of the antibody. Exemplary embodiments include anti-CD39-TGFbRII-BTLA and anti-CD73-TGFbRII-BTLA (e.g., SEQ ID NOs: 425, 422).

In one embodiment, the fusion protein comprises a polypeptide that binds a T cell co-stimulatory molecule and TGFbRII ECD. In some embodiments, the fusion protein is a native T cell co-stimulatory molecule ECD fused to TGFbRII (either N-costimulatory ECD-Fc-TGFbRII ECD-C, or N-TGFbRII ECD-Fc-costimulatory ECD-C). In some embodiments, this fusion protein is selected from: 41BBL-Fc-TGFbRII (e.g., SEQ ID NO: 616); ICOSL-Fc-TGFbRII (e.g., SEQ ID NO: 626); OX40L-Fc-TGFbRII (e.g., SEQ ID NO: 630), TGFbRII-Fc-ICOSL (e.g., SEQ ID NO: 625); TGFbRII-Fc-OX40L (e.g., SEQ ID NO: 629); TGFbRII-Fc-41BBL (e.g., SEQ ID NO: 615). In other embodiments, the fusion protein comprises an antibody or other polypeptide that binds a T cell co-stimulatory molecule fused to TGFbRII. This antibody or polypeptide is preferably an agonist of the T cell co-stimulatory molecule. In some embodiments, this fusion protein is selected from: anti-ICOS-TGFbRII (e.g., SEQ ID NOs: 526, 59); anti-OX40-TGFbRII (e.g., SEQ ID NOs: 514, 97); anti-41BB-TGFbRII (e.g., SEQ ID NOs: 502, 2).

In a further aspect, the fusion protein comprises an antibody that binds a T cell-costimulatory molecule, TGFbRII ECD, and an additional receptor ECD. In one aspect, the TGFbRII ECD is fused to the heavy chain of the antibody and the additional receptor ECD is fused to the light chain of the antibody. In some embodiments, the additional receptor ECD is BTLA ECD, PD1 ECD, TIM3 ECD, SIGLEC10 ECD, or SIRPa ECD. Exemplary embodiments of the invention are anti-OX40-TGFbRII-PD1 (e.g., SEQ ID NOs: 514, 506); anti-OX40-TGFbRII-TIM3 (e.g., SEQ ID NOs: 514, 509); anti-OX40-TGFbRII-SIGLEC10 (e.g., SEQ ID NOs: 514, 507); anti-OX40-TGFbRII-BTLA (e.g., SEQ ID NOs: 514, 511); anti-OX40-TGFbRII-SIRPa (e.g., SEQ ID NOs: 514, 508).

In some embodiments, the fusion protein comprises TGFbRII ECD and a polypeptide that inhibits VEGF/VEGFR signaling. In some embodiments, the fusion protein comprises TGFbRII ECD and anti-VEGFR mAb. In a further embodiment, the fusion protein may additionally comprise a receptor ECD fused to the light chain of the antibody. In some embodiments, the fusion protein is selected from anti-VEGFR-TGFbRII-TIM3 (e.g., SEQ ID NOs: 381, 376); anti-VEGFR-TGFbRII-BTLA (e.g., SEQ ID NOs: 381, 378); anti-VEGFR-TGFbRII-SIGLEC10 (e.g., SEQ ID NOs: 381, 374); anti-VEGFR-TGFbRII-PD1 (e.g., SEQ ID NOs: 381, 373); anti-VEGFR-TGFbRII-SIRPa (e.g., SEQ ID NOs: 381, 375). In some embodiments, the fusion protein comprises TGFbRII ECD and anti-VEGF mAb. In a further embodiment, the fusion protein may additionally comprise a receptor ECD fused to the light chain of the antibody. In some embodiments, the fusion protein is selected from anti-VEGF-TGFbRII-TIM3 (e.g., SEQ ID NOs: 370, 365); anti-VEGF-TGFbRII-SIRPa (e.g., SEQ ID NOs: 370, 364); anti-VEGF-TGFbRII-PD1 (e.g., SEQ ID NOs: 370, 362); anti-VEGF-TGFbRII-BTLA (e.g., SEQ ID NOs: 370, 367); anti-VEGF-TGFbRII-SIGLEC10 (e.g., SEQ ID NOs: 370, 363). In other embodiments, the fusion protein comprises TGFbRII ECD and VEGFR ECD. In one embodiment, this fusion protein is TGFbRII-Fc-VEGFR (e.g., SEQ ID NO: 558). In another embodiment, this fusion protein is VEGFR-Fc-TGFbRII (e.g., SEQ ID NO: 569).

TGFb is a major determinant of TH17 differentiation and function, along with IL-17/IL-17R, IL-6/IL-6R, IL-23/IL-23R. In some embodiments, the fusion protein comprises TGFbRII ECD and an antibody that binds and disables IL-17/IL-17R, IL-23/IL-23R, or IL-6/IL-6R. In some embodiments, the fusion protein is selected from the following: anti-IL17-TGFbRII, anti-IL17R-TGFbRII (e.g., SEQ ID NOs: 334, 63), anti-IL23-TGFbRII (e.g., SEQ ID NOs: 346, 75), anti-IL23R-TGFbRII. In a further embodiment, the fusion protein comprises an additional receptor ECD selected from the following list: SIGLEC10 ECD, SIRPa ECD, BTLA ECD, PD1 ECD, TIM3 ECD.

In some embodiments, the fusion protein comprises TGFbRII ECD and an IL-15R binding fragment of IL-15, or an IL-12R binding fragment of IL-12. Exemplary embodiments include IL15-Fc-TGFbRII (e.g., SEQ ID NO: 590), TGFbRII-Fc-IL15 (e.g., SEQ ID NO: 589), IL12-Fc-TGFbRII (e.g., SEQ ID NO: 588) and TGFbRII-Fc-IL12 (e.g., SEQ ID NO: 587).

In various embodiments, the fusion proteins of the invention counteract PD1/PDL1 in the tumor microenvironment.

In various embodiments, the fusion protein comprises a ligand-binding sequence of an extracellular domain of PD1 (PD1 ECD). In some embodiments, the fusion protein comprises an antibody and PD1 ECD. In one embodiment, the PD1 ECD is fused to the heavy chain of the antibody. In another embodiment, the PD1 ECD is fused to the light chain of the antibody. In a preferred aspect, PD1 ECD is fused to C terminus of antibody heavy chain or light chain. In another aspect, PD1 ECD is fused to N terminus of antibody heavy chain or light chain.

In some embodiments, the fusion protein of the invention comprises a targeting polypeptide that is an antibody. In one embodiment, the antibody is fused to PD1 ECD and additional ligand traps selected from: TIM3 ECD, TGFbRII ECD, BTLA ECD, SIRPa ECD, VEGFR ECD, SIGLEC10 ECD.

In some embodiments, the fusion protein comprises PD1 ECD and a polypeptide that inhibits the interaction between CD47 and SIRPa. In one embodiment, this fusion protein is anti-CD47 mAb fused to PD1 ECD (e.g., anti-CD47-PD1 (e.g., SEQ ID NOs: 387, 22)). In another embodiment, this fusion protein comprises SIRPa ECD and PD1 ECD. In one embodiment, this fusion protein is SIRPa-Fc-PD1 (e.g., SEQ ID NO: 548) or PD1-Fc-SIRPa (e.g., SEQ ID NO: 537).

In a further aspect, the fusion protein comprises PD1 ECD, a polypeptide that inhibits the interaction between CD47 and SIRPa, and another polypeptide that inhibits a T cell co-inhibitory molecule. In some embodiments, this fusion protein comprises anti-CD47 mAb with PD1 ECD fused to the heavy chain or light chain; and the ECD of an immuno-inhibitory molecule fused to the other chain. In a preferred embodiment, the immuno-inhibitory molecule suppresses immune cells via ITIM/ITSMs. In a particular embodiment, this fusion protein is anti-CD47-PD1-TIM3 (e.g., SEQ ID NOs: 387, 386); anti-CD47-PD1-BTLA (e.g., SEQ ID NOs: 387, 388); anti-CD47-PD1-SIGLEC10 (e.g., SEQ ID NOs: 387, 385).

In another aspect, the fusion protein comprises PD1 ECD, anti-CD47 mAb, and VEGFR ECD. In some embodiments, VEGFR ECD is fused to heavy chain of anti-CD47 mAb and PD1 ECD is fused to light chain of anti-CD47 mAb. In one embodiment, this fusion protein is anti-CD47-VEGFR-PD1 (e.g., SEQ ID NOs: 392, 384).

In another aspect, the fusion protein comprises PD1 ECD, SIRPa ECD, and an targeting polypeptide that is an antibody with a heavy chain and light chain. In one aspect, the PD1 ECD is fused to the heavy chain of the antibody and the SIRPa ECD is fused to the light chain of the antibody. Exemplary embodiments include anti-CTLA4-PD1-SIRPa (e.g., SEQ ID NOs: 440, 438), anti-TIM3-PD1-SIRPa (e.g., SEQ ID NOs: 486, 484), anti-PDL1-PD1-SIRPa, anti-EGFR-PD1-SIRPa (e.g., SEQ ID NOs: 225, 223), anti-HER2-PD1-SIRPa (e.g., SEQ ID NOs: 249, 247), anti-EGFRvIII-PD1-SIRPa (e.g., SEQ ID NOs: 237, 235), anti-uPAR-PD1-SIRPa, anti-PSMA-PD1-SIRPa, anti-TGFb-PD1-SIRPa (e.g., SEQ ID NOs: 398, 396), anti-TGFbR-PD1-SIRPa, and anti-GARP-PD1-SIRPa, anti-VEGF-PD1-SIRPa (e.g., SEQ ID NOs: 366, 364), and anti-VEGFR-PD1-SIRPa (e.g., SEQ ID NOs: 377, 375).

In some embodiments, the fusion protein comprises PD1 ECD and a polypeptide that inhibits a T cell co-inhibitory molecule. In specific embodiments, the fusion protein comprises PD1 ECD and an antibody that binds and disables a T cell co-inhibitory molecule. Exemplary embodiments include anti-CTLA4-PD1 (e.g., SEQ ID NOs: 440, 28); anti-PD1-PD1 (e.g., SEQ ID NOs: 452, 101); anti-TIGIT-PD1 (e.g., SEQ ID NOs: 474, 139); anti-TIM3-PD1 (e.g., SEQ ID NOs: 486, 141). Additional exemplary embodiments are anti-PDL1 mAb fused to PD1 ECD where the anti-PDL1 mAb is atezolizumab (SEQ ID NOs: 108, 109); avelumab (SEQ ID NOs: 110, 111); durvalumab (SEQ ID NOs: 112, 113). In a further embodiment, the fusion protein may comprise an additional ECD of an immuno-inhibitory receptor selected from SIGLEC10 ECD, SIRPa ECD, TGFbRII ECD, TIM3 ECD, BTLA ECD, VEGFR ECD.

In some embodiments, the fusion protein comprises an antibody that binds a T cell co-stimulatory molecule fused to PD1. This antibody is preferably an agonist of the T cell co-stimulatory molecule. In some embodiments, this fusion protein is selected from: anti-ICOS-PD1 (e.g., SEQ ID NOs: 522, 59); anti-41BB-PD1 (e.g., SEQ ID NOs: 498, 2); anti-OX40-PD1 (e.g., SEQ ID NOs: 510, 97). In a further embodiment, the fusion protein may comprise an additional ECD of an immuno-inhibitory receptor selected from SIGLEC10 ECD, SIRPa ECD, TGFbRII ECD, TIM3 ECD, BTLA ECD, VEGFR ECD.

In one embodiment, the fusion protein comprises PD1 ECD and a polypeptide that binds either CD39 or CD73. In some embodiments, the fusion protein is an antibody that binds CD39 or CD73 fused to PD1 ECD; for example: anti-CD39-PD1 (e.g., SEQ ID NOs: 429, 18) or anti-CD73-PD1 (e.g., SEQ ID NOs: 421, 24). In some embodiments, the fusion protein comprises an additional receptor ECD fused to the antibody selected from [ECD]. In some embodiments, the fusion protein is selected from: anti-CD73-PD1-SIRPa (e.g., SEQ ID NOs: 421, 419); anti-CD73-PD1-BTLA (e.g., SEQ ID NOs: 421, 422); anti-CD73-PD1-TIM3 (e.g., SEQ ID NOs: 421, 420); anti-CD73-PD1-SIGLEC10 (e.g., SEQ ID NOs: 421, 418).

In some embodiments, the fusion protein comprises a tumor-targeted antibody and PD1 ECD. In some embodiments, this tumor targeted-antibody binds a tumor growth factor, growth factor receptor, or tumor cell surface molecule. In some embodiments, this fusion protein is selected from: anti-EGFR-PD1 (e.g., SEQ ID NOs: 225, 43), anti-HER2-PD1 (e.g., SEQ ID NOs: 249, 55), anti-EGFRvIII-PD1 (e.g., SEQ ID NOs: 237, 47), anti-uPAR-PD1 (e.g., SEQ ID NOs: 269, 162), anti-PSMA-PD1 (e.g., SEQ ID NOs: 276, 121), anti-nectin4-PD1 (e.g., SEQ ID NOs: 261, 160). In a further embodiment, the fusion protein may comprise an additional ECD of an immuno-inhibitory receptor selected from SIGLEC10 ECD, SIRPa ECD, TGFbRII ECD, TIM3 ECD, BTLA ECD, VEGFR ECD.

In some embodiments, the fusion protein comprises PD1 ECD and an antibody that binds and disables IL-17/IL-17R, IL-23/IL-23R, or IL-6/IL-6R. In some embodiments, the fusion protein is selected from: anti-IL17-PD1, anti-IL17R-PD1 (e.g., SEQ ID NOs: 330, 63), anti-IL23-PD1 (e.g., SEQ ID NOs: 342, 75), anti-IL23R-PD1, anti-IL6-PD1, anti-IL6R-PD1 (e.g., SEQ ID NOs: 318, 79). In a further embodiment, the fusion protein may comprise an additional ECD of an immuno-inhibitory receptor selected from SIGLEC10 ECD, SIRPa ECD, TGFbRII ECD, TIM3 ECD, BTLA ECD, VEGFR ECD.

In some embodiments, the fusion protein comprises PD1 ECD and an antibody that binds TGFb, TGFbR, or GARP. Exemplary embodiments of this fusion protein include anti-TGFb-PD1 (e.g., SEQ ID NOs: 398, 133), anti-TGFbR-PD1, and anti-GARP-PD1 (e.g., SEQ ID NOs: 411, 49). In some embodiments, the fusion protein further comprises an additional receptor ECD. Exemplary embodiments of this fusion protein include anti-TGFb-PD1-SIRPa (e.g., SEQ ID NOs: 398, 396); anti-TGFb-PD1-BTLA (e.g., SEQ ID NOs: 398, 399); anti-TGFb-PD1-SIGLEC10 (e.g., SEQ ID NOs: 398, 395); anti-TGFb-PD1-TIM3 (e.g., SEQ ID NOs: 398, 397).

In some embodiments, the fusion protein comprises PD1 ECD and IL-15. In some embodiments, the fusion protein is IL15-Fc-PD1 (e.g., SEQ ID NO: 578), PD1-Fc-IL15 (e.g., SEQ ID NO: 577), IL12-Fc-PD1 (e.g., SEQ ID NO: 576) or PD1-Fc-IL12 (e.g., SEQ ID NO: 575).

In some embodiments, the fusion proteins of the invention comprise a targeting polypeptide that binds PD1 or PDL1 fused to one or more receptor ECDs. These receptor ECDs are preferably selected from: TIM3 ECD, BTLA ECD, VEGFR ECD, TGFbRII ECD, SIRPa ECD, SIGLEC10 ECD. In some embodiments, the targeting polypeptide that binds PD1 or PDL1 is an antibody. Exemplary embodiments include anti-PD1-SIRPa (e.g., SEQ ID NOs: 455, 101); anti-PD1-VEGFR (e.g., SEQ ID NOs: 458, 101); anti-PD1-BTLA (e.g., SEQ ID NOs: 447, 101); anti-PD1-PD1 (e.g., SEQ ID NOs: 452, 101); anti-PD1-TIM3 (e.g., SEQ ID NOs: 457, 101); anti-PD1-SIGLEC10 (e.g., SEQ ID NOs: 454, 101); anti-PD1-TGFbR (e.g., SEQ ID NOs: 456, 101). In another embodiment, the fusion protein comprises antibody that binds PD1 or PDL1 fused to one receptor ECD on heavy chain and another receptor ECD fused on light chain.

In various embodiments, the fusion proteins of the invention counteract TIM3/CEACAM in the tumor microenvironment.

In various embodiments, the fusion protein comprises a ligand-binding sequence of an extracellular domain of TIM-3 (TIM3 ECD). In some embodiments, the fusion protein comprises an antibody and TIM3 ECD. In one embodiment, the TIM3 ECD is fused to the heavy chain of the antibody. In another embodiment, the TIM3 ECD is fused to the light chain of the antibody. In a preferred aspect, TIM3 ECD is fused to C terminus of antibody heavy chain or light chain. In another aspect, TIM3 ECD is fused to N terminus of antibody heavy chain or light chain.

In some embodiments, the fusion protein of the invention comprises a targeting polypeptide that is an antibody. In one embodiment, the antibody is fused to TIM3 ECD and additional ECDs selected from the following: PD1 ECD, TGFbRII ECD, BTLA ECD, SIRPa ECD, VEGFR ECD, SIGLEC10 ECD.

In some embodiments, the fusion protein comprises TIM3 ECD and a polypeptide that inhibits the interaction between CD47 and SIRPa. In one embodiment, this fusion protein is anti-CD47 mAb fused to TIM3 ECD (e.g., anti-CD47-TIM3 (e.g., SEQ ID NOs: 391, 22)). In another embodiment, this fusion protein comprises SIRPa ECD and TIM3 ECD. In one embodiment, this fusion protein is SIRPa-Fc-TIM3 (e.g., SEQ ID NO: 551) or TIM3-Fc-SIRPa (e.g., SEQ ID NO: 562).

In a further aspect, the fusion protein comprises TIM3 ECD, a polypeptide that inhibits the interaction between CD47 and SIRPa, and another polypeptide that inhibits a T cell co-inhibitory molecule. In some embodiments, this fusion protein comprises anti-CD47 mAb with TIM3 ECD fused to the heavy chain or light chain; and the ECD of an immuno-inhibitory molecule fused to the other chain. In a preferred embodiment, the immuno-inhibitory molecule suppresses immune cells via ITIM/ITSMs. In a particular embodiment, this fusion protein is anti-CD47-TIM3-SIGLEC10 (e.g., SEQ ID NOs: 391, 385); anti-CD47-TIM3-PD1 (e.g., SEQ ID NOs: 391, 384); anti-CD47-TIM3-BTLA (e.g., SEQ ID NOs: 391, 388).

In another aspect, the fusion protein comprises TIM3 ECD, anti-CD47 mAb, and VEGFR ECD. In some embodiments, VEGFR ECD is fused to heavy chain of anti-CD47 mAb and TIM3 ECD is fused to light chain of anti-CD47 mAb. In one embodiment, this fusion protein is anti-CD47-VEGFR-TIM3 (e.g., SEQ ID NOs: 392, 386).

In another aspect, the fusion protein comprises TIM3 ECD, SIRPa ECD, and a targeting polypeptide that is an antibody with a heavy chain and light chain. In one aspect, the TIM3 ECD is fused to the heavy chain of the antibody and the SIRPa ECD is fused to the light chain of the antibody. Exemplary embodiments include anti-CTLA4-TIM3-SIRPa (e.g., SEQ ID NOs: 445, 438), anti-PD1-TIM3-SIRPa (e.g., SEQ ID NOs: 457, 450), anti-PDL1-TIM3-SIRPa (e.g., SEQ ID NOs: 467, 461), anti-EGFR-TIM3-SIRPa (e.g., SEQ ID NOs: 230, 223), anti-HER2-TIM3-SIRPa (e.g., SEQ ID NOs: 254, 247), anti-EGFRvIII-TIM3-SIRPa (e.g., SEQ ID NOs: 242, 235), anti-uPAR-TIM3-SIRPa, anti-PSMA-TIM3-SIRPa, anti-TGFb-TIM3-SIRPa (e.g., SEQ ID NOs: 402, 396), anti-TGFbR-TIM3-SIRPa, and anti-GARP-TIM3-SIRPa, anti-VEGF-TIM3-SIRPa (e.g., SEQ ID NOs: 371, 364), and anti-VEGFR-TIM3-SIRPa (e.g., SEQ ID NOs: 382, 375).

In some embodiments, the fusion protein comprises TIM3 ECD and a polypeptide that inhibits a T cell co-inhibitory molecule. In specific embodiments, the fusion protein comprises TIM3 ECD and an antibody that binds and disables a T cell co-inhibitory molecule. Exemplary embodiments include anti-CTLA4-TIM3 (e.g., SEQ ID NOs: 445, 28); anti-TIM3-TIM3 (e.g., SEQ ID NOs: 491, 141); anti-PD1-TIM3 (e.g., SEQ ID NOs: 457, 101); anti-TIGIT-TIM3 (e.g., SEQ ID NOs: 479, 139); anti-PDL1-TIM3 (e.g., SEQ ID NOs: 467, 109). Additional exemplary embodiments are anti-PDL1 mAb fused to TIM3 ECD where the anti-PDL1 mAb is atezolizumab (SEQ ID NOs: 108, 109); avelumab (SEQ ID NOs: 110, 111); durvalumab (SEQ ID NOs: 112, 113). In a further embodiment, the fusion protein may comprise an additional ECD of an immuno-inhibitory receptor selected from SIGLEC10 ECD, SIRPa ECD, TGFbRII ECD, PD1 ECD, BTLA ECD, VEGFR ECD.

In some embodiments, the fusion protein comprises an antibody that binds a T cell co-stimulatory molecule fused to TIM3. This antibody is preferably an agonist of the T cell co-stimulatory molecule. In some embodiments, this fusion protein is selected from: anti-41BB-TIM3 (e.g., SEQ ID NOs: 503, 2); anti-OX40-TIM3 (e.g., SEQ ID NOs: 515, 97); anti-ICOS-TIM3 (e.g., SEQ ID NOs: 527, 59). In a further embodiment, the fusion protein may comprise an additional ECD of an immuno-inhibitory receptor selected from SIGLEC10 ECD, SIRPa ECD, TGFbRII ECD, PD1 ECD, BTLA ECD, VEGFR ECD.

In one embodiment, the fusion protein comprises TIM3 ECD and a polypeptide that binds either CD39 or CD73. In some embodiments, the fusion protein is an antibody that binds CD39 or CD73 fused to TIM3 ECD; for example: anti-CD39-TIM3 (e.g., SEQ ID NOs: 433, 18) or anti-CD73-TIM3 (e.g., SEQ ID NOs: 426, 24). In some embodiments, the fusion protein comprises an additional receptor ECD fused to the antibody selected from [ECD]. In some embodiments, the fusion protein is selected from the following: anti-CD73-TIM3-SIRPa (e.g., SEQ ID NOs: 426, 419); anti-CD73-TIM3-PD1 (e.g., SEQ ID NOs: 426, 417); anti-CD73-TIM3-SIGLEC10 (e.g., SEQ ID NOs: 426, 418); anti-CD73-TIM3-BTLA (e.g., SEQ ID NOs: 426, 422).

In some embodiments, the fusion protein comprises a tumor-targeted antibody and TIM3 ECD. In some embodiments, this tumor targeted-antibody binds a tumor growth factor, growth factor receptor, or tumor cell surface molecule. In some embodiments, this fusion protein is selected from the following: anti-EGFR-TIM3 (e.g., SEQ ID NOs: 230, 43), anti-HER2-TIM3 (e.g., SEQ ID NOs: 254, 55), anti-EGFRvIII-TIM3 (e.g., SEQ ID NOs: 242, 47), anti-uPAR-TIM3 (e.g., SEQ ID NOs: 273, 162), anti-PSMA-TIM3 (e.g., SEQ ID NOs: 280, 121), anti-nectin4-TIM3 (e.g., SEQ ID NOs: 266, 160). In a further embodiment, the fusion protein may comprise an additional ECD of an immuno-inhibitory receptor selected from SIGLEC10 ECD, SIRPa ECD, TGFbRII ECD, PD1 ECD, BTLA ECD, VEGFR ECD.

In some embodiments, the fusion protein comprises TIM3 ECD and an antibody that binds and disables IL-17/IL-17R, IL-23/IL-23R, or IL-6/IL-6R. In some embodiments, the fusion protein is selected from the following: anti-IL17-TIM3, anti-IL17R-TIM3 (e.g., SEQ ID NOs: 335, 63), anti-IL23-TIM3 (e.g., SEQ ID NOs: 347, 75), anti-IL23R-TIM3, anti-IL6-TIM3, anti-IL6R-TIM3 (e.g., SEQ ID NOs: 323, 79). In a further embodiment, the fusion protein may comprise an additional ECD of an immuno-inhibitory receptor selected from SIGLEC10 ECD, SIRPa ECD, TGFbRII ECD, PD1 ECD, BTLA ECD, VEGFR ECD.

In some embodiments, the fusion protein comprises TIM3 ECD and an antibody that binds TGFb, TGFbR, or GARP. Exemplary embodiments of this fusion protein include anti-TGFb-TIM3 (e.g., SEQ ID NOs: 402, 133), anti-TGFbR-TIM3, and anti-GARP-TIM3 (e.g., SEQ ID NOs: 414, 49). In some embodiments, the fusion protein further comprises an additional receptor ECD. Exemplary embodiments of this fusion protein include anti-TGFb-TIM3-SIGLEC10 (e.g., SEQ ID NOs: 402, 395); anti-TGFb-TIM3-BTLA (e.g., SEQ ID NOs: 402, 399); anti-TGFb-TIM3-PD1 (e.g., SEQ ID NOs: 402, 394); anti-TGFb-TIM3-SIRPa (e.g., SEQ ID NOs: 402, 396).

In some embodiments, the fusion protein comprises TIM3 ECD and IL-15. In some embodiments, the fusion protein is IL15-Fc-TIM3 (e.g., SEQ ID NO: 594), TIM3-Fc-IL15 (e.g., SEQ ID NO: 593), IL12-Fc-TIM3 (e.g., SEQ ID NO: 592) or TIM3-Fc-IL12 (e.g., SEQ ID NO: 591).

In some embodiments, the fusion proteins of the invention comprise a targeting polypeptide that binds TIM3 or CEACAM fused to one or more receptor ECDs. These receptor ECDs are preferably selected from the following: PD1 ECD, BTLA ECD, VEGFR ECD, TGFbRII ECD, SIRPa ECD, SIGLEC10 ECD. In some embodiments, the targeting polypeptide that binds TIM3 or CEACAM is an antibody. Exemplary embodiments include anti-TIM3-SIRPa (e.g., SEQ ID NOs: 489, 141); anti-TIM3-TGFbR (e.g., SEQ ID NOs: 490, 141); anti-TIM3-VEGFR (e.g., SEQ ID NOs: 492, 141); anti-TIM3-TIM3 (e.g., SEQ ID NOs: 491, 141); anti-TIM3-SIGLEC10 (e.g., SEQ ID NOs: 488, 141); anti-TIM3-BTLA (e.g., SEQ ID NOs: 481, 141); anti-TIM3-PD1 (e.g., SEQ ID NOs: 486, 141). In another embodiment, the fusion protein comprises antibody that binds TIM3 or CEACAM fused to one receptor ECD on heavy chain and another receptor ECD fused on light chain.

In various embodiments, the fusion proteins of the invention counteract BTLA/HVEM in the tumor microenvironment.

In various embodiments, the fusion protein comprises a ligand-binding sequence of an extracellular domain of B- and T-lymphocyte attenuator (BTLA ECD). In some embodiments, the fusion protein comprises an antibody and BTLA ECD. In one embodiment, the BTLA ECD is fused to the heavy chain of the antibody. In another embodiment, the BTLA ECD is fused to the light chain of the antibody. In a preferred aspect, BTLA ECD is fused to C terminus of antibody heavy chain or light chain. In another aspect, BTLA ECD is fused to N terminus of antibody heavy chain or light chain.

In some embodiments, the fusion protein of the invention comprises a targeting polypeptide that is an antibody. In one embodiment, the antibody is fused to BTLA ECD and additional ligand traps selected from the following: PD1 ECD, TGFbRII ECD, TIM3 ECD, SIRPa ECD, VEGFR ECD, SIGLEC10 ECD.

In some embodiments, the fusion protein comprises BTLA ECD and a polypeptide that inhibits the interaction between CD47 and SIRPa. In one embodiment, this fusion protein is anti-CD47 mAb fused to BTLA ECD (e.g., anti-CD47-BTLA (e.g., SEQ ID NOs: 383, 22)). In another embodiment, this fusion protein comprises SIRPa ECD and BTLA ECD. In one embodiment, this fusion protein is SIRPa-Fc-BTLA (e.g., SEQ ID NO: 547) or BTLA-Fc-SIRPa (e.g., SEQ ID NO: 531).

In a further aspect, the fusion protein comprises BTLA ECD, a polypeptide that inhibits the interaction between CD47 and SIRPa, and another polypeptide that inhibits a T cell co-inhibitory molecule. In some embodiments, this fusion protein comprises anti-CD47 mAb with BTLA ECD fused to the heavy chain or light chain; and the ECD of an immuno-inhibitory molecule fused to the other chain. In a preferred embodiment, the immuno-inhibitory molecule suppresses immune cells via ITIM/ITSMs. In a particular embodiment, this fusion protein is anti-CD47-BTLA-TIM3 (e.g., SEQ ID NOs: 383, 386); anti-CD47-BTLA-PD1 (e.g., SEQ ID NOs: 383, 384); anti-CD47-BTLA-SIGLEC10 (e.g., SEQ ID NOs: 383, 385).

In another aspect, the fusion protein comprises BTLA ECD, anti-CD47 mAb, and VEGFR ECD. In some embodiments, VEGFR ECD is fused to heavy chain of anti-CD47 mAb and BTLA ECD is fused to light chain of anti-CD47 mAb. In one embodiment, this fusion protein is anti-CD47-VEGFR-BTLA (e.g., SEQ ID NOs: 392, 388)

In another aspect, the fusion protein comprises BTLA ECD, SIRPa ECD, and an targeting polypeptide that is an antibody with a heavy chain and light chain. In one aspect, the BTLA ECD is fused to the heavy chain of the antibody and the SIRPa ECD is fused to the light chain of the antibody. Exemplary embodiments include anti-CTLA4-BTLA-SIRPa (e.g., SEQ ID NOs: 435, 438), anti-PD1-BTLA-SIRPa (e.g., SEQ ID NOs: 447, 450), anti-PDL1-BTLA-SIRPa (e.g., SEQ ID NOs: 459, 461), anti-EGFR-BTLA-SIRPa (e.g., SEQ ID NOs: 220, 223), anti-HER2-BTLA-SIRPa (e.g., SEQ ID NOs: 244, 247), anti-EGFRvIII-BTLA-SIRPa (e.g., SEQ ID NOs: 232, 235), anti-uPAR-BTLA-SIRPa, anti-PSMA-BTLA-SIRPa, anti-TGFb-BTLA-SIRPa (e.g., SEQ ID NOs: 393, 396), anti-TGFbR-BTLA-SIRPa, and anti-GARP-BTLA-SIRPa, anti-VEGF-BTLA-SIRPa (e.g., SEQ ID NOs: 361, 364), and anti-VEGFR-BTLA-SIRPa (e.g., SEQ ID NOs: 372, 375).

In some embodiments, the fusion protein comprises BTLA ECD and a polypeptide that inhibits a T cell co-inhibitory molecule. In specific embodiments, the fusion protein comprises BTLA ECD and an antibody that binds and disables a T cell co-inhibitory molecule. Exemplary embodiments include anti-PD1-BTLA (e.g., SEQ ID NOs: 447, 101); anti-PDL1-BTLA (e.g., SEQ ID NOs: 459, 109); anti-TIGIT-BTLA (e.g., SEQ ID NOs: 469, 139); anti-CTLA4-BTLA (e.g., SEQ ID NOs: 435, 28); anti-TIM3-BTLA (e.g., SEQ ID NOs: 481, 141). Additional exemplary embodiments are anti-PDL1 mAb fused to BTLA ECD where the anti-PDL1 mAb is atezolizumab (SEQ ID NOs: 108, 109); avelumab (SEQ ID NOs: 110, 111); durvalumab (SEQ ID NOs: 112, 113). In a further embodiment, the fusion protein may comprise an additional ECD of an immuno-inhibitory receptor selected from SIGLEC10 ECD, SIRPa ECD, TGFbRII ECD, PD1 ECD, TIM3 ECD, VEGFR ECD.

In some embodiments, the fusion protein comprises an antibody that binds a T cell co-stimulatory molecule fused to BTLA. This antibody is preferably an agonist of the T cell co-stimulatory molecule. In some embodiments, this fusion protein is selected from: anti-41BB-BTLA (e.g., SEQ ID NOs: 493, 2); anti-ICOS-BTLA (e.g., SEQ ID NOs: 517, 59); anti-OX40-BTLA (e.g., SEQ ID NOs: 505, 97). In a further embodiment, the fusion protein may comprise an additional ECD of an immuno-inhibitory receptor selected from SIGLEC10 ECD, SIRPa ECD, TGFbRII ECD, PD1 ECD, TIM3 ECD, VEGFR ECD.

In one embodiment, the fusion protein comprises BTLA ECD and a polypeptide that binds either CD39 or CD73. In some embodiments, the fusion protein is an antibody that binds CD39 or CD73 fused to BTLA ECD; for example: anti-CD39-BTLA (e.g., SEQ ID NOs: 428, 18) or anti-CD73-BTLA (e.g., SEQ ID NOs: 416, 24). In some embodiments, the fusion protein comprises an additional receptor ECD fused to the antibody selected from [ECD]. In some embodiments, the fusion protein is selected from the following: anti-CD73-BTLA-SIGLEC10 (e.g., SEQ ID NOs: 416, 418); anti-CD73-BTLA-SIRPa (e.g., SEQ ID NOs: 416, 419); anti-CD73-BTLA-TIM3 (e.g., SEQ ID NOs: 416, 420); anti-CD73-BTLA-PD1 (e.g., SEQ ID NOs: 416, 417).

In some embodiments, the fusion protein comprises a tumor-targeted antibody and BTLA ECD. In some embodiments, this tumor targeted-antibody binds a tumor growth factor, growth factor receptor, or tumor cell surface molecule. In some embodiments, this fusion protein is selected from the following: anti-EGFR-BTLA (e.g., SEQ ID NOs: 220, 43), anti-HER2-BTLA (e.g., SEQ ID NOs: 244, 55), anti-EGFRvIII-BTLA (e.g., SEQ ID NOs: 232, 47), anti-uPAR-BTLA (e.g., SEQ ID NOs: 268, 162), anti-PSMA-BTLA (e.g., SEQ ID NOs: 275, 121), anti-nectin4-BTLA (e.g., SEQ ID NOs: 256, 160). In a further embodiment, the fusion protein may comprise an additional ECD of an immuno-inhibitory receptor selected from SIGLEC10 ECD, SIRPa ECD, TGFbRII ECD, PD1 ECD, TIM3 ECD, VEGFR ECD.

In some embodiments, the fusion protein comprises BTLA ECD and an antibody that binds and disables IL-17/IL-17R, IL-23/IL-23R, or IL-6/IL-6R. In some embodiments, the fusion protein is selected from the following: anti-IL17-BTLA, anti-IL17R-BTLA (e.g., SEQ ID NOs: 325, 63), anti-IL23-BTLA (e.g., SEQ ID NOs: 337, 75), anti-IL23R-BTLA, anti-IL6-BTLA, anti-IL6R-BTLA (e.g., SEQ ID NOs: 313, 79). In a further embodiment, the fusion protein may comprise an additional ECD of an immuno-inhibitory receptor selected from SIGLEC10 ECD, SIRPa ECD, TGFbRII ECD, PD1 ECD, TIM3 ECD, VEGFR ECD.

In some embodiments, the fusion protein comprises BTLA ECD and an antibody that binds TGFb, TGFbR, or GARP. Exemplary embodiments of this fusion protein include anti-TGFb-BTLA (e.g., SEQ ID NOs: 393, 133), anti-TGFbR-BTLA, and anti-GARP-BTLA (e.g., SEQ ID NOs: 410, 49). In some embodiments, the fusion protein further comprises an additional receptor ECD. Exemplary embodiments of this fusion protein include anti-TGFb-BTLA-TIM3 (e.g., SEQ ID NOs: 393, 397); anti-TGFb-BTLA-PD1 (e.g., SEQ ID NOs: 393, 394); anti-TGFb-BTLA-SIRPa (e.g., SEQ ID NOs: 393, 396); anti-TGFb-BTLA-SIGLEC10 (e.g., SEQ ID NOs: 393, 395).

In some embodiments, the fusion protein comprises BTLA ECD and IL-15. In some embodiments, the fusion protein is IL15-Fc-BTLA (e.g., SEQ ID NO: 574), BTLA-Fc-IL15 (e.g., SEQ ID NO: 573), IL12-Fc-BTLA (e.g., SEQ ID NO: 572) or BTLA-Fc-IL12 (e.g., SEQ ID NO: 571).

In some embodiments, the fusion proteins of the invention comprise a targeting polypeptide that binds BTLA or HVEM fused to one or more receptor ECDs. These receptor ECDs are preferably selected from the following: PD1 ECD, TIM-3 ECD, VEGFR ECD, TGFbRII ECD, SIRPa ECD, SIGLEC10 ECD. In some embodiments, the targeting polypeptide that binds BTLA or HVEM is an antibody. In another embodiment, the fusion protein comprises antibody that binds BTLA or HVEM fused to one receptor ECD on heavy chain and another receptor ECD fused on light chain.

In various embodiments, the fusion proteins of the invention counteract SIRPa/CD47 in the tumor microenvironment.

In various embodiments, the fusion protein comprises a ligand-binding sequence of an extracellular domain of SIRPa (SIRPa ECD). In some embodiments, the fusion protein comprises an antibody and SIRPa ECD. In one embodiment, the SIRPa ECD is fused to the heavy chain of the antibody. In another embodiment, the SIRPa ECD is fused to the light chain of the antibody. In a preferred aspect, SIRPa ECD is fused to C terminus of antibody heavy chain or light chain. In another aspect, SIRPa ECD is fused to N terminus of antibody heavy chain or light chain.

In some embodiments, the fusion protein of the invention comprises a targeting polypeptide that is an antibody. In one embodiment, the antibody is fused to SIRPa ECD and additional ligand traps selected from the following: TIM3 ECD, TGFbRII ECD, BTLA ECD, PD1 ECD, VEGFR ECD, SIGLEC10 ECD.

In some embodiments, the fusion protein comprises SIRPa ECD and a polypeptide that inhibits a T cell co-inhibitory molecule. In specific embodiments, the fusion protein comprises SIRPa ECD and an antibody that binds and disables a T cell co-inhibitory molecule. Exemplary embodiments include anti-PD1-SIRPa (e.g., SEQ ID NOs: 455, 101); anti-TIM3-SIRPa (e.g., SEQ ID NOs: 489, 141); anti-CTLA4-SIRPa (e.g., SEQ ID NOs: 443, 28); anti-TIGIT-SIRPa (e.g., SEQ ID NOs: 477, 139); anti-PDL1-SIRPa (e.g., SEQ ID NOs: 465, 109). Additional exemplary embodiments are anti-PDL1 mAb fused to SIRPa ECD where the anti-PDL1 mAb is atezolizumab (SEQ ID NOs: 108, 109); avelumab (SEQ ID NOs: 110, 111); durvalumab (SEQ ID NOs: 112, 113). In a further embodiment, the fusion protein may comprise an additional ECD of an immuno-inhibitory receptor selected from SIGLEC10 ECD, PD1 ECD, TGFbRII ECD, TIM3 ECD, BTLA ECD, VEGFR ECD.

In some embodiments, the fusion protein comprises an antibody that binds a T cell co-stimulatory molecule fused to SIRPa. This antibody is preferably an agonist of the T cell co-stimulatory molecule. In some embodiments, this fusion protein is selected from: anti-ICOS-SIRPa (e.g., SEQ ID NOs: 525, 59); anti-OX40-SIRPa (e.g., SEQ ID NOs: 513, 97); anti-41BB-SIRPa (e.g., SEQ ID NOs: 501, 2). In a further embodiment, the fusion protein may comprise an additional ECD of an immuno-inhibitory receptor selected from SIGLEC10 ECD, PD1 ECD, TGFbRII ECD, TIM3 ECD, BTLA ECD, VEGFR ECD.

In one embodiment, the fusion protein comprises SIRPa ECD and a polypeptide that binds either CD39 or CD73. In some embodiments, the fusion protein is an antibody that binds CD39 or CD73 fused to SIRPa ECD; for example: anti-CD39-SIRPa (e.g., SEQ ID NOs: 431, 18) or anti-CD73-SIRPa (e.g., SEQ ID NOs: 424, 24). In some embodiments, the fusion protein comprises an additional receptor ECD fused to the antibody selected from [ECD]. In some embodiments, the fusion protein is selected from the following: anti-CD73-SIRPa-BTLA (e.g., SEQ ID NOs: 424, 422); anti-CD73-SIRPa-PD1 (e.g., SEQ ID NOs: 424, 417); anti-CD73-SIRPa-SIGLEC10 (e.g., SEQ ID NOs: 424, 418); anti-CD73-SIRPa-TIM3 (e.g., SEQ ID NOs: 424, 420).

In some embodiments, the fusion protein comprises a tumor-targeted antibody and SIRPa ECD. In some embodiments, this tumor targeted-antibody binds a tumor growth factor, growth factor receptor, or tumor cell surface molecule. In some embodiments, this fusion protein is selected from the following: anti-EGFR-SIRPa (e.g., SEQ ID NOs: 228, 43), anti-HER2-SIRPa (e.g., SEQ ID NOs: 252, 55), anti-EGFRvIII-SIRPa (e.g., SEQ ID NOs: 240, 47), anti-uPAR-SIRPa (e.g., SEQ ID NOs: 271, 162), anti-PSMA-SIRPa (e.g., SEQ ID NOs: 278, 121), anti-nectin4-SIRPa (e.g., SEQ ID NOs: 264, 160). In a further embodiment, the fusion protein may comprise an additional ECD of an immuno-inhibitory receptor selected from SIGLEC10 ECD, PD1 ECD, TGFbRII ECD, TIM3 ECD, BTLA ECD, VEGFR ECD.

In some embodiments, the fusion protein comprises SIRPa ECD and an antibody that binds and disables IL-17/IL-17R, IL-23/IL-23R, or IL-6/IL-6R. In some embodiments, the fusion protein is selected from the following: anti-IL17-SIRPa, anti-IL17R-SIRPa (e.g., SEQ ID NOs: 333, 63), anti-IL23-SIRPa (e.g., SEQ ID NOs: 345, 75), anti-IL23R-SIRPa, anti-IL6-SIRPa, anti-IL6R-SIRPa (e.g., SEQ ID NOs: 321, 79). In a further embodiment, the fusion protein may comprise an additional ECD of an immuno-inhibitory receptor selected from SIGLEC10 ECD, PD1 ECD, TGFbRII ECD, TIM3 ECD, BTLA ECD, VEGFR ECD.

In some embodiments, the fusion protein comprises SIRPa ECD and an antibody that binds TGFb, TGFbR, or GARP. Exemplary embodiments of this fusion protein include anti-TGFb-SIRPa (e.g., SEQ ID NOs: 401, 133), anti-TGFbR-SIRPa, and anti-GARP-SIRPa (e.g., SEQ ID NOs: 413, 49). In some embodiments, the fusion protein further comprises an additional receptor ECD. Exemplary embodiments of this fusion protein include anti-TGFb-SIRPa-BTLA (e.g., SEQ ID NOs: 401, 399); anti-TGFb-SIRPa-TIM3 (e.g., SEQ ID NOs: 401, 397); anti-TGFb-SIRPa-PD1 (e.g., SEQ ID NOs: 401, 394); anti-TGFb-SIRPa-SIGLEC10 (e.g., SEQ ID NOs: 401, 395).

In some embodiments, the fusion protein comprises SIRPa ECD and IL-15. In some embodiments, the fusion protein is IL15-Fc-SIRPa (e.g., SEQ ID NO: 586), SIRPa-Fc-IL15 (e.g., SEQ ID NO: 585), IL12-Fc-SIRPa (e.g., SEQ ID NO: 584) or SIRPa-Fc-IL12 (e.g., SEQ ID NO: 583).

In some embodiments, the fusion proteins of the invention comprise a targeting polypeptide that binds SIRPa or CD47 fused to one or more receptor ECDs. These receptor ECDs are preferably selected from the following: TIM3 ECD, BTLA ECD, VEGFR ECD, TGFbRII ECD, PD1 ECD, SIGLEC10 ECD. In some embodiments, the targeting polypeptide that binds SIRPa or CD47 is an antibody. Exemplary embodiments include anti-CD47-TGFbR (e.g., SEQ ID NOs: 390, 22); anti-CD47-SIGLEC10 (e.g., SEQ ID NOs: 389, 22); anti-CD47-VEGFR (e.g., SEQ ID NOs: 392, 22); anti-CD47-BTLA (e.g., SEQ ID NOs: 383, 22); anti-CD47-PD1 (e.g., SEQ ID NOs: 387, 22); anti-CD47-TIM3 (e.g., SEQ ID NOs: 391, 22). In another embodiment, the fusion protein comprises antibody that binds SIRPa or CD47 fused to one receptor ECD on heavy chain and another receptor ECD fused on light chain.

In various embodiments, the fusion proteins of the invention counteract SIGLEC10/CD24 in the tumor microenvironment.

In various embodiments, the fusion protein comprises a ligand-binding sequence of an extracellular domain of SIGLEC10 (SIGLEC10 ECD). In some embodiments, the fusion protein comprises an antibody and SIGLEC10 ECD. In one embodiment, the SIGLEC10 ECD is fused to the heavy chain of the antibody. In another embodiment, the SIGLEC10 ECD is fused to the light chain of the antibody. In a preferred aspect, SIGLEC10 ECD is fused to C terminus of antibody heavy chain or light chain. In another aspect, SIGLEC10 ECD is fused to N terminus of antibody heavy chain or light chain.

In some embodiments, the fusion protein of the invention comprises a targeting polypeptide that is an antibody. In one embodiment, the antibody is fused to SIGLEC10 ECD and additional ligand traps selected from the following: TIM3 ECD, TGFbRII ECD, BTLA ECD, PD1 ECD, VEGFR ECD, SIRPa ECD.

In some embodiments, the fusion protein comprises SIGLEC10 ECD and a polypeptide that inhibits the interaction between CD47 and SIRPa. In one embodiment, this fusion protein is anti-CD47 mAb fused to SIGLEC10 ECD (e.g., anti-CD47-SIGLEC10 (e.g., SEQ ID NOs: 389, 22)). In another embodiment, this fusion protein comprises SIRPa ECD and SIGLEC10 ECD. In one embodiment, this fusion protein is SIRPa-Fc-SIGLEC10 (e.g., SEQ ID NO: 549) or SIGLEC10-Fc-SIRPa (e.g., SEQ ID NO: 543).

In a further aspect, the fusion protein comprises SIGLEC10 ECD, a polypeptide that inhibits the interaction between CD47 and SIRPa, and another polypeptide that inhibits a T cell co-inhibitory molecule. In some embodiments, this fusion protein comprises anti-CD47 mAb with SIGLEC10 ECD fused to the heavy chain or light chain; and the ECD of an immuno-inhibitory molecule fused to the other chain. In a preferred embodiment, the immuno-inhibitory molecule suppresses immune cells via ITIM/ITSMs. In a particular embodiment, this fusion protein is anti-CD47-SIGLEC10-TIM3 (e.g., SEQ ID NOs: 389, 386); anti-CD47-SIGLEC10-BTLA (e.g., SEQ ID NOs: 389, 388).

In another aspect, the fusion protein comprises SIGLEC10 ECD, anti-CD47 mAb, and VEGFR ECD. In some embodiments, VEGFR ECD is fused to heavy chain of anti-CD47 mAb and SIGLEC10 ECD is fused to light chain of anti-CD47 mAb. In one embodiment, this fusion protein is anti-CD47-VEGFR-SIGLEC10 (e.g., SEQ ID NOs: 392, 385).

In another aspect, the fusion protein comprises SIGLEC10 ECD, PD1 ECD, and an targeting polypeptide that is an antibody with a heavy chain and light chain. In one aspect, the SIGLEC10 ECD is fused to the heavy chain of the antibody and the PD1 ECD is fused to the light chain of the antibody. Exemplary embodiments include anti-CTLA4-SIGLEC10-PD1 (e.g., SEQ ID NOs: 442, 436), anti-TIM3-SIGLEC10-PD1 (e.g., SEQ ID NOs: 488, 482), anti-PDL1-SIGLEC10-PD1, anti-EGFR-SIGLEC10-PD1 (e.g., SEQ ID NOs: 227, 221), anti-HER2-SIGLEC10-PD1 (e.g., SEQ ID NOs: 251, 245), anti-EGFRvIII-SIGLEC10-PD1 (e.g., SEQ ID NOs: 239, 233), anti-uPAR-SIGLEC10-PD1, anti-PSMA-SIGLEC10-PD1, anti-TGFb-SIGLEC10-PD1 (e.g., SEQ ID NOs: 400, 394), anti-TGFbR-SIGLEC10-PD1, and anti-GARP-SIGLEC10-PD1, anti-VEGF-SIGLEC10-PD1 (e.g., SEQ ID NOs: 368, 362), and anti-VEGFR-SIGLEC10-PD1 (e.g., SEQ ID NOs: 379, 373).

In some embodiments, the fusion protein comprises SIGLEC10 ECD and a polypeptide that inhibits a T cell co-inhibitory molecule. In specific embodiments, the fusion protein comprises SIGLEC10 ECD and an antibody that binds and disables a T cell co-inhibitory molecule. Exemplary embodiments include anti-PDL1-SIGLEC10 (e.g., SEQ ID NOs: 463, 109); anti-TIM3-SIGLEC10 (e.g., SEQ ID NOs: 488, 141); anti-PD1-SIGLEC10 (e.g., SEQ ID NOs: 454, 101); anti-CTLA4-SIGLEC10 (e.g., SEQ ID NOs: 442, 28); anti-TIGIT-SIGLEC10 (e.g., SEQ ID NOs: 476, 139). Additional exemplary embodiments are anti-PDL1 mAb fused to SIGLEC10 ECD where the anti-PDL1 mAb is atezolizumab (SEQ ID NOs: 108, 109); avelumab (SEQ ID NOs: 110, 111); durvalumab (SEQ ID NOs: 112, 113). In a further embodiment, the fusion protein may comprise an additional ECD of an immuno-inhibitory receptor selected from SIRPa ECD, PD1 ECD, TGFbRII ECD, TIM3 ECD, BTLA ECD, VEGFR ECD.

In some embodiments, the fusion protein comprises an antibody that binds a T cell co-stimulatory molecule fused to SIGLEC10. This antibody is preferably an agonist of the T cell co-stimulatory molecule. In some embodiments, this fusion protein is selected from: anti-41BB-SIGLEC10 (e.g., SEQ ID NOs: 500, 2); anti-OX40-SIGLEC10 (e.g., SEQ ID NOs: 512, 97); anti-ICOS-SIGLEC10 (e.g., SEQ ID NOs: 524, 59). In a further embodiment, the fusion protein may comprise an additional ECD of an immuno-inhibitory receptor selected from SIRPa ECD, PD1 ECD, TGFbRII ECD, TIM3 ECD, BTLA ECD, VEGFR ECD.

In one embodiment, the fusion protein comprises SIGLEC10 ECD and a polypeptide that binds either CD39 or CD73. In some embodiments, the fusion protein is an antibody that binds CD39 or CD73 fused to SIGLEC10 ECD; for example: anti-CD39-SIGLEC10 (e.g., SEQ ID NOs: 430, 18) or anti-CD73-SIGLEC10 (e.g., SEQ ID NOs: 423, 24). In some embodiments, the fusion protein comprises an additional receptor ECD fused to the antibody selected from [ECD]. In some embodiments, the fusion protein is selected from the following: anti-CD73-SIGLEC10-PD1 (e.g., SEQ ID NOs: 423, 417); anti-CD73-SIGLEC10-TIM3 (e.g., SEQ ID NOs: 423, 420); anti-CD73-SIGLEC10-SIRPa (e.g., SEQ ID NOs: 423, 419); anti-CD73-SIGLEC10-BTLA (e.g., SEQ ID NOs: 423, 422).

In some embodiments, the fusion protein comprises a tumor-targeted antibody and SIGLEC10 ECD. In some embodiments, this tumor targeted-antibody binds a tumor growth factor, growth factor receptor, or tumor cell surface molecule. In some embodiments, this fusion protein is selected from the following: anti-EGFR-SIGLEC10 (e.g., SEQ ID NOs: 227, 43), anti-HER2-SIGLEC10 (e.g., SEQ ID NOs: 251, 55), anti-EGFRvIII-SIGLEC10 (e.g., SEQ ID NOs: 239, 47), anti-uPAR-SIGLEC10 (e.g., SEQ ID NOs: 270, 162), anti-PSMA-SIGLEC10 (e.g., SEQ ID NOs: 277, 121), anti-nectin4-SIGLEC10 (e.g., SEQ ID NOs: 263, 160). In a further embodiment, the fusion protein may comprise an additional ECD of an immuno-inhibitory receptor selected from SIRPa ECD, PD1 ECD, TGFbRII ECD, TIM3 ECD, BTLA ECD, VEGFR ECD.

In some embodiments, the fusion protein comprises SIGLEC10 ECD and an antibody that binds IL-17/IL-17R, IL-23/IL-23R, or IL-6/IL-6R. In a preferred embodiment, the antibody interrupts the interaction between the ligand and receptor. In some embodiments, the fusion protein is selected from the following: anti-IL17-SIGLEC10, anti-IL17R-SIGLEC10 (e.g., SEQ ID NOs: 332, 63), anti-IL23-SIGLEC10 (e.g., SEQ ID NOs: 344, 75), anti-IL23R-SIGLEC10, anti-IL6-SIGLEC10, anti-IL6R-SIGLEC10 (e.g., SEQ ID NOs: 320, 79). In a further embodiment, the fusion protein may comprise an additional ECD of an immuno-inhibitory receptor selected from SIRPa ECD, PD1 ECD, TGFbRII ECD, TIM3 ECD, BTLA ECD, VEGFR ECD.

In some embodiments, the fusion protein comprises SIGLEC10 ECD and an antibody that binds TGFb, TGFbR, or GARP. Exemplary embodiments of this fusion protein include anti-TGFb-SIGLEC10 (e.g., SEQ ID NOs: 400, 133), anti-TGFbR-SIGLEC10, and anti-GARP-SIGLEC10 (e.g., SEQ ID NOs: 412, 49). In some embodiments, the fusion protein further comprises an additional receptor ECD. Exemplary embodiments of this fusion protein include anti-TGFb-SIGLEC10-TIM3 (e.g., SEQ ID NOs: 400, 397); anti-TGFb-SIGLEC10-PD1 (e.g., SEQ ID NOs: 400, 394); anti-TGFb-SIGLEC10-SIRPa (e.g., SEQ ID NOs: 400, 396); anti-TGFb-SIGLEC10-BTLA (e.g., SEQ ID NOs: 400, 399).

In some embodiments, the fusion protein comprises SIGLEC10 ECD and IL-15 ECD or IL-12 ECD. In some embodiments, the fusion protein is IL15-Fc-SIGLEC10 (e.g., SEQ ID NO: 582), SIGLEC10-Fc-IL15 (e.g., SEQ ID NO: 581), IL12-Fc-SIGLEC10 (e.g., SEQ ID NO: 580) or SIGLEC10-Fc-IL12 (e.g., SEQ ID NO: 579).

In some embodiments, the fusion proteins of the invention comprise a targeting polypeptide that binds SIGLEC10 or CD47 fused to one or more receptor ECDs. These receptor ECDs are preferably selected from the following: TIM3 ECD, BTLA ECD, VEGFR ECD, TGFbRII ECD, PD1 ECD, SIRPa ECD. In some embodiments, the targeting polypeptide that binds SIGLEC10 or CD47 is an antibody. Exemplary embodiments include anti-CD47-TGFbR (e.g., SEQ ID NOs: 390, 22); anti-CD47-SIGLEC10 (e.g., SEQ ID NOs: 389, 22); anti-CD47-VEGFR (e.g., SEQ ID NOs: 392, 22); anti-CD47-BTLA (e.g., SEQ ID NOs: 383, 22); anti-CD47-PD1 (e.g., SEQ ID NOs: 387, 22); anti-CD47-TIM3 (e.g., SEQ ID NOs: 391, 22). In another embodiment, the fusion protein comprises antibody that binds SIGLEC10 or CD47 fused to one receptor ECD on heavy chain and another receptor ECD fused on light chain.

In one embodiment the present invention provides methods of treating cancer by administering agent(s) that counteract multiple ligand/receptor interactions that promote tumor angiogenesis and/or immune dysfunction in the tumor microenvironment (TME).

The molecules of the invention may be used for the treatment of cancer. Further, the molecules of the invention may be used in conjunction or in combination with any other type of therapy including surgery, chemotherapy, radiation therapy, targeted small molecules, anti-angiogenic therapy or immunotherapy. Immunotherapy may include any immuno-oncologic drug selected from a broad range of agents, including antibodies, vaccines, adjuvant therapies, cytokines, oncolytic viruses, bispecific molecules, and cellular therapies. In a specific embodiment, the molecules of the invention may be administered to a subject in combination with (Chimeric Antigen Receptor (CAR) T cell therapy. In various aspects, the molecules of the invention may be administered in combination with one or more different molecules of the invention. In various aspects, the molecules of the invention may be administered prior to, concurrently, sequentially, and/or following another therapy. In various aspects, the molecules of the invention may be administered in a composition with any other therapeutic agent or molecule of the invention.

In some embodiments, molecules of the invention alone or in combination with other therapies counteract immune tolerance in the tumor microenvironment. In some embodiments, molecules of the invention alone or in combination with other therapies counteract angiogenesis in the tumor microenvironment.

In various embodiments, a subject may be administered one or more molecules from one or more of the following types of fusion proteins of the invention: fusion protein comprising an anti-VEGF antibody or anti-VEGFR antibody or VEGF-binding sequence of VEGFR ECD; fusion protein comprising anti-TGFb antibody or TGFb-binding sequence of TGFbR ECD; fusion protein comprising anti-PD1 antibody or anti-PDL1 antibody or PD1-binding sequence of PD1 ECD; fusion protein comprising anti-BTLA antibody or anti-HVEM antibody or HVEM-binding sequence of BTLA ECD; fusion protein comprising anti-CEACAM antibody or anti-TIM3 antibody or CEACAM-binding sequence of TIM3 ECD; fusion protein comprising anti-CD47 antibody or anti-SIRPa antibody or CD47-binding sequence of SIRPa ECD; fusion protein comprising anti-CD24 antibody or anti-SIGLEC10 antibody or CD24-binding sequence of SIGLEC10 ECD.

In one embodiment, the present invention provides method of treating a subject having a disease or disorder comprising administering to the subject a fusion protein of the invention. In certain embodiments the patient has cancer.

In various embodiments, a subject may be administered one or more fusion proteins described in the invention. In some embodiments, the fusion proteins comprise one or more of TGFbRII ECD, VEGFR ECD, PD1 ECD, BTLA ECD, SIRPa ECD, TIM3 ECD, and SIGLEC10 ECD.

In various embodiments, a subject may be administered one or more fusion proteins described in the invention in combination with a polypeptide that disables an immune cell inhibitory molecule or T cell co-inhibitory molecule (e.g., CTLA-4, BTLA, TIM-3, CEACAM1, or CEACAM-5, TIGIT, PVRIG, VISTA, VSIG8, LAG-3, CD47, SIRPa, CD24, SIGLEC10, or LILRB1). In some embodiments, this polypeptide is an antibody. In other embodiments, the polypeptide is a fusion protein comprising the ECD of a T cell co-inhibitory molecule. In some embodiments, this polypeptide may be PD1-Fc, TIM3-Fc, or BTLA-Fc. In some embodiments, the polypeptide may be an anti-PD¹/anti-PDL1 mAb. Exemplary such antibodies are anti-PD1 (e.g., nivolumab, pembrolizumab) and anti-PDL1 (e.g., durvalumab, avelumab, atezolizumab).

In various embodiments, a subject may be administered one or more fusion proteins described in the invention in combination with a second fusion protein described in the invention. In various aspects, this second fusion protein disables a T cell co-inhibitory molecule. In some embodiments, this second fusion protein comprises BTLA ECD, TIM-3 ECD, or PD-1 ECD.

In various embodiments, a subject may be administered one or more fusion proteins described in the invention in combination with an antibody or fusion protein that activates an T cell co-stimulatory molecule (e.g., OX40, 41BB, ICOS, GITR, HVEM, CD27, CD40, CD30, DNAM). In some embodiments, the fusion protein comprises the ECD of a T cell co-stimulatory ligand (e.g., OX40L, 41BBL, ICOSL, GITRL, LIGHT, CD70, CD40L, CD30L) that binds a T cell co-stimulatory receptor as an agonist.

In various embodiments, a subject may be administered one or more fusion proteins described in the invention in combination with an inhibitor of TGFb/TGFbR. In some aspects, this TGFb/TGFbR inhibitor is selected from: a-TGFb antibody; a-TGFbR antibody; TGFbRII ECD containing fusion protein (e.g. TGFbRIIecd-Fc); TGFbR TKI (e.g. galunisertib); anti-GARP antibody; anti-LAP antibody; ALT comprising TGFbRII ECD; ALT that inhibits TGFb/TGFbR; fusion proteins described in this invention that inhibit TGFb/TGFbR.

In various embodiments, a subject may be administered one or more fusion proteins described in the invention in combination with an inhibitor of VEGF/VEGFR. In some aspects, the VEGF/VEGFR inhibitor may be selected from: anti-VEGF antibody (e.g., bevacizumab), anti-VEGFR antibody (e.g. ramucirumab), VEGFR kinase inhibitor (e.g., sunitinib, sorafenib, axitinib, cabozantinib, regorafenib, pazopanib, vandetanib, lenvatenib), or VEGFR ECD-Fc fusion protein (e.g., aflibercept).

In various embodiments, a subject may be administered one or more fusion proteins described in the invention in combination with an inhibitor of TH17 differentiation and/or function. In some aspects, this agent is an inhibitor of the interaction between IL23/IL23R, IL1/IL1R, IL6/IL6R, or IL17/IL17R. In some aspects, this agent is selected from: anti-IL17R antibody, anti-IL17 antibody, anti-IL17 nanobody; anti-IL6R antibody, anti-IL6 antibody; IL23 antibody, anti-IL23R antibody; anti-IL1R antibody, anti-IL1 antibody. In other aspects, this agent is a fusion protein described in the invention that inhibits IL23/IL23R, IL1/IL1R, IL6/IL6R, IL17/IL17R.

In various embodiments, a subject may be administered one or more fusion proteins described in the invention in combination with an immunocytokine or cytokine fusion protein comprising an active ligand or ligand fragment of IL12 or IL15.

In various embodiments, a subject may be administered one or more fusion proteins described in the invention in combination with a polypeptide that binds a tumor cell- or tumor antigen, tumor growth factor or growth factor receptor. In one aspect, this polypeptide is an antibody. In another aspect, this polypeptide is conjugated to a cytotoxic compound. In a further aspect, this polypeptide is an ADC. In a further aspect, this polypeptide is an ALT-DC.

In various embodiments, a subject may be administered one or more fusion proteins described in the invention in combination with a chimeric antigen receptor T cell (CAR T cell)

In various embodiments, a subject may be administered one or more fusion proteins described in the invention in combination with an immunotherapeutic agent.

In various embodiments, a subject may be administered one or more fusion proteins described in the invention in combination with an inhibitor of tumor cell signaling that promotes tumor cell survival, proliferation, invasion, and/or metastases; tumor angiogenesis; or immune dysfunction in the TME.

In various embodiments, a subject may be administered one or more fusion proteins described in the invention in combination with a chemotherapeutic or cytotoxic agent, a DNA repair inhibitor or PARP inhibitor, a tumor vaccine or viriolytic agent; or ionizing radiation.

### BRIEF DESCRIPTION OF THE DRAWINGS

FIG. 1 is a Schematic representation of anti-PDL1-BTLA ECD and anti-PDL1-TGFbRII ECD-BTLA ECD.

FIGS. 2A-2B show antibody-ligand traps containing BTLA ECD localize to HVEM-expressing cells and simultaneously counteract BTLA-mediated suppression & promote HVEM-mediated activation of T cells. BTLA ligation by HVEM inhibits T cell activation via SHP-1-mediated inhibition of CD28 and CD3z signaling. HVEM ligation by LIGHT or BTLA (in trans) promotes T cell activation. The antibody ligand traps of the invention comprising a BTLA ECD which binds HVEM, thereby disrupting its interaction with both BTLA and CD160. In addition, ligation of T cell HVEM by BTLA ECD of the ALT may promote HVEM-mediated costimulatory signals for T cell activationPD-1 ligation by PD-1 ligands (PD-L1 or PD-L2) inhibits T cell activation via SHP-2-mediated inhibition of CD28 signaling. The interaction of PD-L1 with PD-1 can be disrupted by antibodies targeting either PD-L1 or PD-1, or a PD1 ECD that binds both PD-L1 and PD-L2. Antibody ligand traps comprising a BTLA ECD fused to an antibody that specifically binds PD-L1, or PD-1 can simultaneously inhibit PD-L1/PD-1 and HVEM/BTLA induced SHP1/2 mediated suppression of CD28 and CD3 signaling. As such, these molecules of the invention can counteract both HVEM/BTLA and PD-L1 mediated immune suppression in the tumor environment, thereby enhancing antitumor immune responses.

FIGS. 3A-3B show the characterization of a-PDL1-BTLA ECD, a-PDL1-TGFbRII and a-PDL1-TGFbRII ECD-BTLA ECD. 3A: SDS-PAGE under reducing (R) and non-reducing (NR) conditions was used to compare the full-length (FL), heavy chain (HC) and light chain (LC) of anti-PDL1 (atezolizumab), anti-PDL1-BTLA ECD and anti-PDL1-TGFbRII ECD-BTLA ECD. 3B: SEC-HPLC analysis of anti-PDL1 (atezolizumab), anti-PDL1-BTLA ECD and anti-PDL1-TGFbRII; ECD-BTLA ECD (>99% monomericity).

FIGS. 4A-4D show the target binding ability of a-PDL1-BTLA ECD, a-PDL1-TGFbRII and a-PDL1-TGFbRII ECD-BTLA ECD. 4A: Standard ELISA showing the ability of anti-PDL1 (atezolizumab), anti-PDL1-BTLA ECD and anti-PDL1-TGFbRII ECD-BTLA ECD to bind PD-L1. 4B: Standard ELISA showing the ability of anti-PDL1-TGFbRII ECD and anti-PDL1-TGFbRII ECD-BTLA ECD to bind TGFb. 4C: Standard ELISA showing the ability of; anti-PDL1-BTLA ECD and anti-PDL1-TGFbRII ECD-BTLA ECD to bind the BTLA ligand HVEM. 4D: ELISA showing the ability of anti-PDL1-BTLA ECD and anti-PDL1-TGFbRII ECD-BTLA ECD to simultaneously bind PD-L1 and the BTLA ligand HVEM.

FIGS. 5A-5C show anti-PDL1, and anti-PDL1-BTLA ECD activity. 5A: Ability of anti-PDL1, anti-PDL1-BTLA ECD to elicit antitumor immunity and inhibit the growth of syngeneic B16-F10 tumors in C57BL/6 muMt-mice. 5B: Ability of anti-PDL1, anti-PDL1-BTLA ECD to elicit antitumor immunity and inhibit the growth of cancers was examined in human immune reconstituted NSG mice bearing human WiDR-colorectal cancer cells. 5C: Ability of anti-PDL1, anti-PDL1-BTLA ECD, anti-PDL1-TGFbRII ECD and anti-PDL1-BTLA ECD-TGFbRII ECD to elicit antitumor immunity and inhibit the growth of cancers was examined in human immune reconstituted NSG mice bearing human BXPC3-pancreatic cancer cells.

FIGS. 6A-6C show a-VEGF-PD1 activity. 6A: Mice are treated with mAbs 24 h prior to the radiotracer injection. 6B: CD3+ are counted in immunohistochemistry images of tumors in control group. anti-VEGF group, or anti-VEGF-PD1 ECD group. 6C: NSG mice immune reconstituted with tumor-matched HLA A2+ human CD34+ HSC and bearing KRAS mutant D-MUT1 human colorectal cancer tumor xenografts were treated (5 mg/kg i.p. weekly) with vehicle alone (untreated control) or the following antibodies (either alone or in combination), as indicated: with either vehicle alone (untreated control) or the following antibodies: a-VEGF-PD1 ECD; a-VEGF (bevacizumab.

FIGS. 7A-7C show a-VEGF-TGFbRII-PD1 activity. 7A: Structure of anti-VEGF-TGFbRII-PD1. anti-VEGF binds VEGF, TGFbRII binds TGFb, PD1 binds PD-L1 and PD-L2. 7B: NSG mice immune were reconstituted with tumor-matched HLA A2+ human CD34+ HSC followed by subcutaneous implantation of WiDR tumor xenografts. 7C: NSG mice immune were reconstituted with tumor-matched HLA A2+ human CD34+ HSC followed by subcutaneous implantation of BXPC3 tumor xenografts.

FIGS. 8A-8C show a-VEGF-TGFbRII-PD1 activity. 8A: Standard ELISA showing the ability of anti-VEGF (bevacizumab), anti-VEGF-TGFbRII-PD1 to bind VEGF. 8B: Standard ELISA showing the ability of anti-VEGF-TGFbRII-PD1 to bind TGFb. 8C: Standard ELISA showing the ability of anti-VEGF-TGFbRII-PD1 to bind TGFb as well as IgG-TGFbRII.

FIGS. 9A-9C shows anti-HER2-TGFbRII and anti-HER2-PD1 activity. 9A: SDS-PAGE under reducing (R) and non-reducing (NR) conditions was used to compare the full-length (FL), heavy chain (HC) and light chain (LC) of anti-HER2-TGFβRIIECD, anti-HER2-PD1 ECD, and anti-HER2 (Trastuzumab). 9B: The ability of anti-HER2-TGFβRII to simultaneously bind HER2 and TGF-β1 was evaluated by a ‘double-sandwich’ ELISA wherein anti-HER2-TGFβRIIECD was added to HER2-Fc coated plates, followed by rhTGF-β1 (1 ng/ml) that was detected by a biotinylated anti-huTGF-β1 antibody. 9C: The ability of anti-HER2-TGFβRII to bind TGF-β1 was also evaluated by competition immunoassays.

FIGS. 10A-10B show anti-HER2-TGFbRII activity. 10A: Residual tumors in TrastuzumabR BT-474 (BT-474-TR) F2 tumor-bearing mice following treatment with anti-HER2-TGFβRII were significantly smaller (mean±SEM=31.7±6.5) than those in F2 mice treated with anti-HER2 mAb (mean±SEM=453.9±121.4) (p=0.003). 10B: Serum was collected from TrastuzumabR BT-474 tumor-bearing mice.

FIGS. 11A-11C show a-EGFR-TGFbRII activity. 11A: Human tumor xenografts were generated by mammary fat pad implantation of the MDA-MB-231-Luc (D3H2LN) TNBC line in female immune deficient NOG mice (NOD/Shi-scid IL-2rgnull). 11B: Immune deficient NSG mice (NOD/Shi-scid IL-2rgnull; 6-8 weeks old) were irradiated at 200 cGy and rested for 6-8 h, followed by adoptive transfer of human CD34+ cells (7×104/mouse) from a normal donor (HLA-matched to the D-MUT1 line)(ALLCELLS). 11C: Nude mice were inoculated subcutaneously with a PDX of human HNSCC (SCC harvested from the floor of the mouth).

FIGS. 12A-12B show anti-PD1-TIM3 and anti-PDL1-TIM3 activity. 12A: (top) Schematic of anti-PD1-TIM3 ECD and anti-PDL1-TIM3 ECD. Anti-PDL1-TIM3 ECD was designed to target both PD-L1 and TIM3 ligands by fusing the C-terminus of the heavy chain of an anti-PDL1 antibody with a ligand binding sequence of the extracellular domain of TIM3 (TIM3 ECD) via a flexible linker peptide, (GGGGS)3. 12B: (top) a-PD1-TIM3 ECD (430.3±29.9) inhibits tumor growth significantly more effectively than untreated control (908.2±40.3), a-PD-1 (824.0±38.3), IgG-TIM3 ECD (825.1±79.0) or the combination of IgG-TIM3 ECD and a-PD1 (884.7±97.4) (p<0.0001). 12 B: (bottom) a-PDL1-TIM3 ECD (226.4±71.4) inhibits tumor growth more effectively than a-PDL1 (617.5±144.3), a-TIM-3 (640.9±99.6) or the combination of a-TIM-3 and a-PDL1 mAbs (653.0±59.8) (p<0.001).

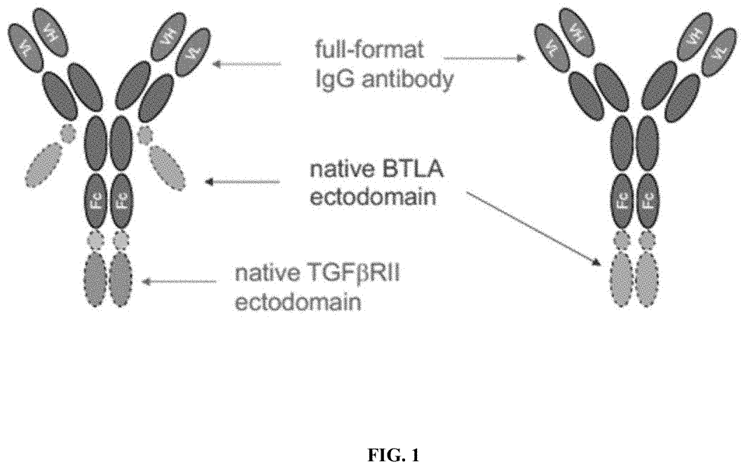

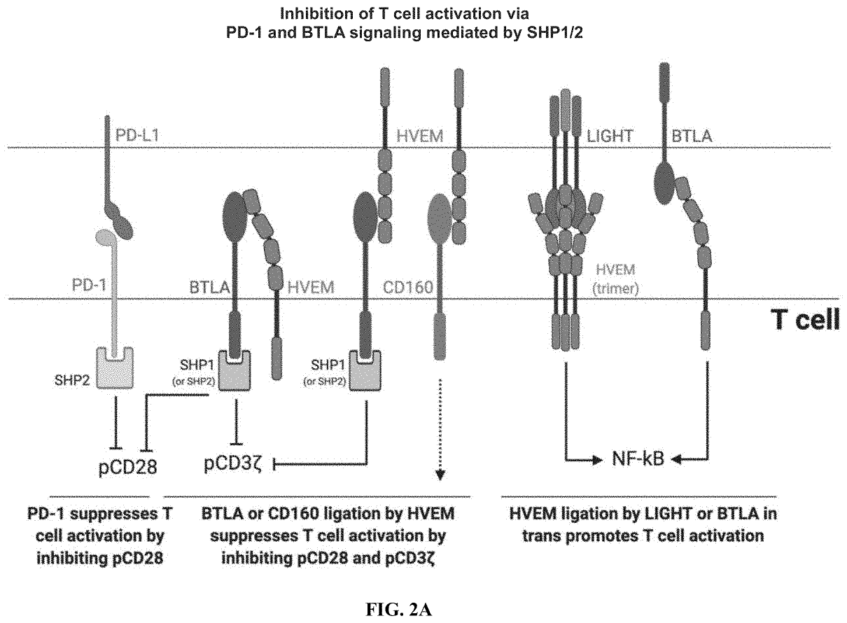

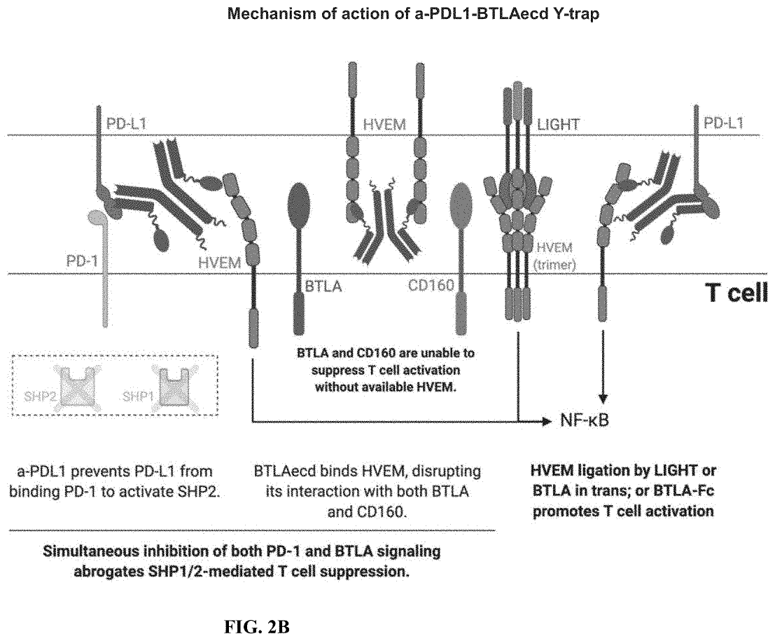

### DETAILED DESCRIPTION OF THE INVENTION

The present invention is based on the seminal discovery that fusion proteins comprising at least one ligand binding sequence of the extracellular domain of a protein and a targeting moiety are effective at treating various diseases and disorders.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In some embodiments, the fusion proteins of the invention comprise a targeting polypeptide to which one or more ECDs are fused. “X” is a molecule that is specifically bound by the targeting polypeptide. “Y” is a ligand-binding sequence of an extracellular domain, or fragment thereof. In some embodiments, the fusion proteins of the invention have the structure “anti-{X}-{Y}”, where the ligand-binding sequence of the extracellular domain Y is fused to the targeting polypeptide. In some embodiments, the targeting polypeptide is an antibody that comprises at least one heavy chain and one light chain. In some embodiments, Y is fused to the C terminus of the light chain of the antibody. In other embodiments, Y is fused to the C terminus of the heavy chain of the antibody. In some embodiments, Y is fused to the N terminus of the light chain of the antibody. In other embodiments, Y is fused to the N terminus of the heavy chain of the antibody.

In some embodiments, the targeting polypeptide is an antibody or other polypeptide comprising a heavy chain and light chain connected by one or more disulfide bonds. “X” is a molecule that is specifically bound by this targeting polypeptide. “Y” is a ligand-binding sequence of an extracellular domain, or fragment thereof “Z” is a ligand-binding sequence of a different extracellular domain, or fragment thereof. In some embodiments, the fusion proteins of the invention have the structure “anti-{X}-{Y}-{Z}”, where Y and Z are fused to the polypeptide that binds X. In some embodiments, Y is fused to the C terminus of the heavy chain of the antibody and Z is fused to the C terminus of the light chain of the antibody. In other embodiments, Y is fused to the C terminus of the light chain of the antibody and Z is fused to the C terminus of the heavy chain of the antibody. In some embodiments, Y is fused to the N terminus of the heavy chain of the antibody and Z is fused to the N terminus of the light chain of the antibody. In other embodiments, Y is fused to the N terminus of the light chain of the antibody and Z is fused to the N terminus of the heavy chain of the antibody.

In some embodiments, the ECD is fused to the C terminus of the antibody heavy chain or light chain. In other cases, the ECD may be fused to the N terminus of the antibody heavy chain or light chain.

In some embodiments, the fusion proteins comprise two ECDs (ECD #1, ECD #2) fused together. Fusion proteins that comprise two ECDs fused together may be referred to as “ECD-ECD”s in this invention. In some embodiments, the fusion protein additionally comprises a Fc domain. In some embodiments, the fusion protein additionally comprises a linker. In some embodiments, the structure of the fusion protein is N (terminus)-ECD #1-ECD #2-C (terminus). In other embodiments, the structure is N-ECD #2-ECD #1-C. In other embodiments, the structure is N-ECD #1-linker-ECD #2-C or N-ECD #2-linker-ECD #1-C. In other embodiments, the structure is N-ECD #1-Fc-ECD #2-C or N-ECD #2-linker-ECD #1-C. In other embodiments, the structure is N-ECD #1-Fc-linker-ECD #2-C, or N-ECD #2-Fc-linker-ECD #1-C.

In one aspect, two or more ECDs may be fused in serial. In one aspect, the fusion protein comprises a targeting polypeptide and two or more ECDs are fused in serial on the same chain of the targeting polypeptide. In one aspect, two or more ECDs are fused in serial on the light chain of a targeting polypeptide that is an antibody. In one aspect, the two or more ECDs are connected by linkers. For example, the light chain of a fusion protein of the invention might be N terminus-antibody light chain-linker-ECD #1-linker-ECD #1-C terminus. In another aspect, the ECD may be fused to both the N and C terminii of the same chain. For example, the light chain of a fusion protein of the invention might be N terminus-ECD #1-linker-antibody light chain-linker-ECD #1-C terminus.

In one aspect, component parts of the fusion proteins of the invention are fused via a flexible linker. In a further aspect, the flexible linker comprises the polypeptide sequence (GGGGS)n where n is between 1 and 10. In another aspect, the flexible linker is selected from the following list: (GGGGS)3 (SEQ ID NO: 200), (GGGGS)4 (SEQ ID NO: 201), waldo1999 (SEQ ID NO: 202), bird1988-1 (SEQ ID NO: 203), bird1988-2 (SEQ ID NO: 204). In one aspect, a linker may be used to fuse an ECD to a targeting polypeptide. In another aspect, a linker may be used to fuse one ECD to another. In another aspect, a linker may be used to fuse an ECD to the C terminus of the CH3 region of the heavy chain of an Fc polypeptide. In another aspect, the linker is rigid. In a further aspect, the rigid linker is selected from the following list: (EAAAK)3 (SEQ ID NO: 205), A(EAAAK)3A (SEQ ID NO: 206), (AP)7 (SEQ ID NO: 207).

“Reeptor ECD”, “extracellular domain”, “ECD”, “ECD”, “ligand trap” and “LT” are used interchangeably in this invention. In any case where any of these terms are used, they may also refer to any ligand-binding sequence of the extracellular domain or fragment thereof in question.

The ECD(s), or “ligand traps”, of the fusion protein enable one or more of the following functions: (1) sequestration of ligands/cytokines that contribute to angiogenesis in the tumor microenvironment; (2) sequestration of ligands/cytokines that promote tumor cell survival, proliferation, invasion, and/or metastasis; (3) sequestration of ligands/cytokines that contribute to tumor-induced immune tolerance or immune dysfunction; (4) activation of a T cell co-stimulatory molecule; (5) inhibition of a T cell co-inhibitory molecule; (5) preferential localization to the target tissue/cell microenvironment expressing its cognate ligand(s).

In some embodiments, an ECD of the invention may be modified in one or more of the following ways: (1) substitution or deletion of residues that are not necessary for ligand binding, (2) substitution of residues to remove N-linked glycosylation sites, (3) substitution, addition, or deletion of residues to increase affinity to one or more of its cognate ligands, (4) substitution, addition, or deletion of residues to improve the expression of the fusion protein, (5) substitution, addition, or deletion of residues to allow for site-specific conjugation of drug conjugates, (6) substitution, addition, or deletion of residues to decrease the specificity of the ligand trap to one or more of its cognate ligands while maintaining or increasing its specificity to other cognate ligands, (7) fusion of non-continuous domains of the same ECD, (8) fusion of domains from different isoforms of the same ECD, (9) fusion of domains from different members of the same ECD family. In some embodiments, any of these modifications refer to the same ECD if they result in a sequence that maintains 90%, 95%, 98%, or 99% sequence identity to a ligand-binding sequence of the ECD.

N-glycosylation occurs at the following consensus sites: NX1S or NX1T, where X1 is any amino acid that is not proline. N is asparagine, S is serine, and T is threonine. More rarely, N-glycosylation can occur at NX2C where N is asparagine, X2 is any amino acid, and C is cysteine. In some embodiments, one or more N-glycosylation consensus sites may be mutated to reduce glycosylation of the ligand trap. In some embodiments, hypoglycosylation of the ligand trap is achieved by mutation of an asparagine residue in a N-glycosylation consensus sequence to another polar amino acid (i.e., serine, threonine, or glutamine). In other embodiments, hypoglycosylation of the ligand trap is achieved by mutation of an asparagine residue in a N-glycosylation consensus sequence to alanine.

In various embodiments, the fusion proteins of the invention comprise one or more of the following ECDs: (1) a ligand-binding sequence of an extracellular domain of TGFbR (e.g., TGFbRII ECD), or fragment thereof. In one aspect, this ECD binds TGFb1, TGFb2, and/or TGFb3; (2) a ligand-binding sequence of an extracellular domain of PD-1 (e.g., PD1 ECD), or fragment thereof. In one aspect, this ECD binds PD-L1 and/or PD-L2. In one embodiment, this ligand trap has one or more amino acid substitutions which increase its affinity for PD-L1 and/or PD-L2; (3) a ligand-binding sequence of an extracellular domain of VEGFR (e.g., VEGFR1, VEGFR2, VEGFR3), or fragment thereof, or a fusion of VEGF-binding sequences of one or more VEGFR extracellular domains (e.g., VEGFR1 domain 2 fused to VEGFR2 domain 3). In one aspect, this ECD binds VEGFA, VEGFB, VEGFC, and/or PIGF; (4) a ligand-binding sequence of an extracellular domain of TIM-3 (e.g., TIM3 ECD), or fragment thereof, or a hypoglycosylated variant of TIM-3, or fragment thereof. In one aspect, this ECD binds CEACAM1, CEACAM5, phosphatidyl-serine, and/or Galectin-9; (5) a ligand-binding sequence of an extracellular domain of SIRPa (e.g., SIRPa-ECD), or fragment thereof; or a hypoglycosylated variant of SIRPa, or fragment thereof. In one aspect, this ECD binds CD47; (6) a ligand-binding sequence of an extracellular domain of B- and T-lymphocyte attenuator (BTLA ECD) or fragment thereof, or a hypoglycosylated variant of BTLA or fragment thereof. In one aspect, this ECD binds herpesvirus entry mediator (HVEM); (7) a ligand-binding sequence of an extracellular domain of SIGLECi0 or fragment thereof, or a hypoglycosylated variant of SIGLECi0 or fragment thereof. In one aspect, this ECD binds CD24.

In one embodiment, the fusion protein comprises a ligand-binding sequence of an extracellular domain of TGFbR (e.g., TGFbRII ECD) or fragment thereof to bind and disable TGFb in the target cell microenvironment. In some embodiments, the TGFbR ECD may be a ligand-binding sequence of TGFbRII ECD. In some embodiments, the TGFbR ECD may be a fusion of domains from TGFbRII and TGFbRIII. In some embodiments, the TGFbR ECD may be selected from the following list: SEQ ID NOS: 177; 178; 179; 180. TGFβ inhibits the expression of cytotoxic effector molecules in immune cells and suppresses their ability to induce antibody-mediated ADCC of tumor cells. TGFb results in a significant decrease in their expression of several cytotoxic effector molecules, including granzyme B, Apo2L/TRAIL, CD95L/FasL, and IFN-γ. TGFb inhibits T cell-mediated antitumor immunity. TGF suppresses the expression of interferon-gamma (IFNgamma), restricts the differentiation of TH1 cells, attenuates the activation and cytotoxic function of CD8+ effector cells, and inhibits the development of central memory T cells. Most significantly, TGFb induces the differentiation of regulatory T cells (Tregs), a sub-population of immunosuppressive CD4+ T cells that express the CD25 and FOXP3. TGFb induces the expression of FOXP3, the signature transcription factor that determines and maintains the functional program of the Treg lineage. Besides inhibiting NK cells and regulating T cell lineage determination and function, TGFb also promotes the polarization of pro-tumorigenic M2 macrophages which secrete high levels of TGFb, IL-6, and IL-10. As such, TGF counteracts the ability of tumor-targeted antibody to induce adaptive antitumor immunity via Fc-FcR mediated antigen cross-presentation by DCs. In one aspect, the ligand-binding sequence of TGFbRII ECD that is fused to a targeting antibody binds and traps TGFb1, TGFb2, and/or TGFb3. As such, a fusion protein of the invention comprising TGFbRII ECD thus uniquely sequesters TGFb1, TGFb2, and TGFb3 in the tumor or target microenvironment in such a way that all immunosuppressive, angiogenic, and tumor-promoting effects of TGFb are blocked in the target or tumor cell microenvironment.

In one embodiment, the fusion protein comprises a ligand-binding sequence of an extracellular domain of Programmed Death-1 (PD-1 ECD) or fragment thereof to bind and disable PD-1 ligands (PD-L1 or PD-L2) in the target cell microenvironment. Upregulation of Programmed death-1 ligands (PD-L1, PD-L2) is a major common denominator of immune tolerance via suppression of natural killer (NK) cell and T cell-mediated antitumor immunity. Engagement of PD-1 by its ligands [PD-L1 (B7-H1); PD-L2 (B7-DC)], inhibits the proliferation, survival, and function of T cells, and cooperates with TGF-b to promote the differentiation and function of Tregs. Tumor cell expression of PD-1 ligands in the tumor microenvironment inhibits activation of tumor infiltrating T cells via interaction of PD-L1 and PD-L2 with either PD-1 or B7. PD1 ECD completely sequesters both PD-L1 and PD-L2, preventing either ligand from interacting with PD-1 on T cells/NK cells or CD80/86 on DCs. This complete inhibition of all PD-1 ligand activity is not recapitulated by either anti-PD1 or anti-PDL1 antibodies: anti-PDL1 has no effect on PD-L2 signaling; and anti-PD1 has no effect on PD-L1/2 binding to CD80/86 on DCs. In one aspect, the ligand-binding sequence of PD1 ECD that is fused to a targeting antibody binds and traps PD-L1 and/or PD-L2. In one aspect, the PD-1ECD fused to the targeting antibody comprises a PD-1ECD sequence incorporating specific mutations in residues to increase the affinity to PD-L1 and/or PD-L2 (high affinity PD-1ECD). As such, the fusion protein comprising a fused PD-1ECD uniquely sequesters both PD-L1 and PD-L2 in the tumor microenvironment in such a way that all immunosuppressive effects of PD-L1/2 are blocked in the target cell microenvironment. In some embodiments, the PD1 ECD may be selected from the following list: SEQ ID NOS: 169; 170; 171

In one embodiment, the fusion protein comprises a ligand-binding sequence of an extracellular domain of Vascular endothelial growth factor receptor (VEGFR1 and/or VEGFR2) or fragment thereof to bind and disable VEGF in the target cell microenvironment. VEGF induces angiogenesis and immune suppression in the tumor microenvironment. VEGF is a high-affinity ligand for receptor tyrosine kinases VEGFR1, VEGFR2, and VEGFR3. VEGFA mRNA is overexpressed in most human tumors, where its expression correlates with invasiveness, increased vascular density, metastasis, tumor recurrence and poor prognosis. Tumors frequently harbor intrinsic genetic alterations that activate signaling pathways (RAS/MAPK, PI3K/AKT, or STAT3) leading to hypoxianti-inducible factor 1 (HIF-1)-mediated induction of VEGF expression in the tumor microenvironment. In addition, specific cytokines (IL-6, TGFb, IL-17) play a key role in increasing local levels of VEGF in the tumor immune microenvironment. VEGFA secreted by tumor cells and surrounding stroma stimulates the proliferation and survival of endothelial cells, leading to the formation of new blood vessels. In addition to secreting VEGF, tumors themselves express VEGF-R1 and VEGF-R2. VEGF promotes tumor migration and metastasis, via autocrine/paracrine stimulation of angiogenesis, cancer stem cell renewal and stability. VEGF also plays an angiogenesis-independent role in cancer immune evasion. VEGF inhibits anti-tumor immunity on multiple levels including promotion and expansion of inhibitory immune cells, such as myeloid-derived suppressor cells (MDSC), suppression of dendritic cell (DC) maturation, mitigation of effector T cell responses, and alteration of lymphocyte development and trafficking. In one aspect, the fusion protein comprises a ligand-binding sequence of VEGFR ECD that binds VEGFR ligands, (VEGF-A, VEGF-B) and optionally placental growth factor (PIGF), and prevents these ligands from binding their endothelial receptors, VEGFR-1 and VEGFR-2. In one aspect, the fusion protein comprises the vascular endothelial growth factor (VEGF) binding portions from the extracellular domains of human VEGF receptors 1 and 2. In one aspect, the VEGF trap comprises Ig domain 2 from VEGFR1, fused to Ig domain 3 from VEGFR2. In one embodiment, the fusion protein comprises amino acids 103-204 of VEGFR1 fused to amino acids 206-308 of VEGFR2. In one embodiment, the fusion protein comprises the same domains of VEGFR1 and VEGFR2 as aflibercept. In some embodiments, the VEGFR ECD may be selected from the following list: SEQ ID NOS: 184; 185; 186

In one embodiment, the fusion protein comprises a ligand-binding sequence of an extracellular domain of TIM-3 (TIM3 ECD) or fragment thereof to bind and disable TIM-3 ligands (CEACAM1, CEACAM5) in the target cell microenvironment. TIM-3 and CEACAM1 are transmembrane proteins expressed on activated T cells, and their expression is induced by cytokines involved in effector T cell differentiation (e.g., IFNgamma). CEACAM1 suppresses T cell activity via homodimerization of its extracellular domains in cis or trans (CEACAM1/CEACAM1). This homophilic interaction is required for phosphorylation of CEACAM1 cytoplasmic domain ITIMs and recruitment of the SHP-1/SHP-2 tyrosine phosphatases which effect proximal suppression of TCR signaling and inhibition of effector functions, including T cell proliferation, TH1 cytokine production and cytotoxicity. CEACAM1 also suppresses TCR signaling via binding TIM-3 along their shared signature “cleft.” The interaction of CEACAM1 with TIM-3 induces phosphorylation-mediated release of Bat3 and loss of Lck-mediated TCR signaling. CEACAM family members (CEACAM1, CEACAM5) are highly expressed in many tumor types, especially gastrointestinal adenocarcinomas (e.g., pancreatic cancer, colorectal cancer). Besides the cis interactions on T cells involving CEACAM1/TIM-3, CEACAM1 and CEACAM5 on tumor cells are able to induce exhaustion via trans interactions with CEACAM1 or TIM-3 on T cells. CEACAM family members also suppress the effector functions of innate immune cells. In one aspect, the ALT comprises a ligand-binding sequence of TIM-3ECD that binds CEACAM1 and/or CEACAM5. In another aspect, the TIM-3ECD sequence may contain mutations in amino acid residues that are normally glycosylated in order to reduce glycosylation at these sites. In one aspect, the fused TIM3 ECD serves as a decoy to sequester TIM-3 ligands (CEACAM1 and CEACAM5 on T cells and/or tumor cells), thereby disrupting cis and trans homodimeric and heterodimeric interactions of the CEACAM axis that lead to T cell exhaustion. Decoration of fusion protein-targeted cells in the TME with a decoy TIM-3ECD sequesters CEACAM family members by competing with native TIM-3 to bind the FG-CC′ cleft of CEACAM, enabling native TIM-3 on T cells to remain unligated and able to sustain Bat3/Lck-mediated T cell activation. In addition to counteracting T cell exhaustion, in another aspect the fusion protein comprising TIM3 ECD can simultaneously binds a tumor cell and CEACAM-1 on a T cell, thereby recruiting and sustaining tumor-reactive cytotoxic T cells in the tumor cell microenvironment.

In one embodiment, the ligand trap may be a ligand-binding sequence of an extracellular domain of TIM-3 (TIM3 ECD) or fragment thereof that is hypoglycosylated or deglycosylated (hypoglycosylated TIM-3 ECD or deglycosylated TIM-3 ECD). In some embodiments, any N-glycosylation consensus site of TIM3 ECD is mutated to reduce glycosylation of TIM3 ECD. In some embodiments, hypoglycosylation of the TIM3 ECD is achieved by mutations at one or more of the following sites: N78, V79, T80, N151, L152, and/or T153.

In some embodiments, hypoglycosylation of the TIM3 ECD is achieved by mutation of a threonine residue in a N-glycosylation consensus site to isoleucine. In some embodiments, hypoglycosylation of the TIM3 ECD is achieved by a T801 mutation.

In some embodiments, the TIM3 ECD or TIM3 ECD variant may be selected from the following list: SEQ ID NOS: 181; 182; 183.

In one embodiment, the fusion protein comprises a ligand-binding sequence of an extracellular domain of B- and T-lymphocyte attenuator (BTLA-ECD) or fragment thereof. In one aspect, this ligand trap (BTLA ECD) binds herpesvirus entry mediator (HVEM). In one aspect, the BTLA ECD comprises sequence that binds at least the cysteine-rich domain 1 region (CRD1) of HVEM. In another aspect the BTLA ECD sequence that binds HVEM enables HVEM to recruit the signaling molecules TNFR-associated factor (TRAF1, TRAF2, TRAF3 and/or TRAF5, activation of nuclear factor-κB (NF-κB) and/or activator protein 1 (AP1) transcription factors. In another aspect the BTLA ECD sequence promotes the co-stimulation or survival of immune cells or T-cells. In one aspect, the BTLA ECD sequence does not interfere with the interaction between HVEM and LIGHT. In another aspect, the BTLA ECD sequence binds HVEM and promotes HVEM-mediated activation of NF-κB. In another aspect, the BTLA ECD sequence lacks the C-terminal cytoplasmic domain that recruits SHP-1 or SHP-2. In this aspect, BTLA ECD binds HVEM and prevents HVEM from binding native BTLA, thereby blocking the ability of HVEM to bind native BTLA and consequent BTLA-mediated SHP1/SHP2-dependent inhibition of TCR signaling. In addition to blocking inhibitory signaling, the BTLA ECD sequence may activate HVEM-mediated activation and/or survival of T cells.

In one embodiment, the ligand trap may be a ligand-binding sequence of an extracellular domain of BTLA (BTLA ECD) or fragment thereof that is hypoglycosylated or deglycosylated (hypoglycosylated BTLA ECD or deglycosylated BTLA ECD). In some embodiments, any N-glycosylation consensus site of BTLA ECD is mutated to reduce glycosylation of BTLA ECD. In some embodiments, hypoglycosylation of the BTLA ECD is achieved by mutations at one or more of the following sites: N76, G77, T78, N95, 196, S97, N111, G112, and/or S113.

In some embodiments, the BTLA ECD or BTLA ECD variant may be selected from the following list: SEQ ID NOS: 165; 166; 167; 168

In one embodiment, the fusion protein comprises a ligand-binding sequence of an extracellular domain of SIRPa (SIRPa-ECD) or fragment thereof to bind and disable CD47. The ligand-binding N-terminal domain of SIRPa (SIRPa d1) binds CD47 through the loops at the end of the domain. CD47 possesses an unusual disulphide link between the IgSF domain and one of the loops between the transmembrane regions, which is required for optimal binding of SIRPa. In one embodiment, the fusion protein comprises a CD47 binding sequence of the extracellular region or IgSF domain of SIRPa (SIRPa ECD). In one aspect, the fusion protein comprises a CD47 binding sequence of the N-terminal domain of SIRPa (SIRPa dl). In one aspect, the SIRPa ECD may be mutated to have higher affinity for CD47. In some embodiments, the SIRPa-ECD may be selected from the following list: SEQ ID NOS: 173; 174; 175; 176

In one embodiment, the fusion protein comprises a ligand-binding sequence of an extracellular domain of Sialic Acid Binding Ig Like Lectin 10 (SIGLEC10 ECD) or fragment thereof to bind and disable CD24. SIGLEC10 is highly expressed by tumor-associated macrophages; and tumors often express high levels of CD24. Endogenous SIGLEC10 contains two ITIM domains on its cytoplasmic tail, and ligation of SIGLEC10 by CD24 results in SHP1/SHP2-mediated inhibitory signaling that prevent the macrophage from phagocytosing the CD24-expressing tumor cell. In some embodiments, the SIGLEC10 ECD may be selected from the following list: SEQ ID NOS: 172.

In some embodiments, the fusion protein comprises a ligand-binding sequence of the extracellular domain of a T cell co-stimulatory molecule or a fragment thereof. In one aspect, this ECD is capable of binding its cognate T cell co-inhibitory molecule and promote T cell activation. In some embodiments, this T cell co-stimulatory molecule is selected from OX40L, 41BBL, ICOSL, GITRL, LIGHT, CD70, CD40L, CD30L.

In some embodiments, the fusion protein may comprise a ligand-binding sequence of the extracellular domain of CD160 or a fragment thereof.

In various embodiments, the fusion proteins of the invention comprise a targeting polypeptide (TP) in addition to one or more ligand traps. The targeting polypeptide comprises a polypeptide which specifically binds a component of a tumor cell, tumor microenvironment, tumor associated growth factor or receptor, tumor associated cytokine or receptor, tumor associated T lymphocyte, T cell co-stimulatory or inhibitory molecule, immune cell, pathogen, or pathogen-associated cell.

In some embodiments, this targeting polypeptide is an antibody. In such cases, the fusion protein of the invention may be referred to as an “antibody-ligand trap”, or “ALT”, which are used interchangeably.

In one aspect, the targeting polypeptide comprises an antigen-binding domain of an immunoglobulin, antibody, bispecific or multispecific antibody, antibody fragment, single chain variable fragment (scFv), bivalent or multivalent scFv, Affimer, a ligand-binding sequence from the extracellular domain (ECD) of a receptor, or Fc-containing polypeptide. In certain aspects, the targeting polypeptide is an antibody.

In the case that the targeting polypeptide is a bispecific antibody (bsAb), it may be an obligate or non-obligate bsAb. In some embodiments, one of the targets of the bsAb is CD3.

In some embodiments, the bsAb is bivalent in a 1+1 format (i.e., one binding site for each target). In a further embodiment, the bispecific antibody may be a tandem VHH nanobody fusion, tandem scFvs (e.g., BiTE), DART, diabody, F(ab)2, or scFv-Fab fusion. In another embodiment, the bispecific antibody may comprise two or more asymmetric chains: for example, hetero heavy chains with forced knob-and-hole HL pairing, hetero heavy chains with CrossMab VH/VL swapped domains, hetero heavy chains with CrossMAB CH1/CL swapped domains, DART-Fc, LP-DART, or half-life-extended BiTE.

In other embodiments, the bsAb is trivalent in a 1+2 format (i.e., 1 binding site for one target and 2 binding sites for the other target). In a further embodiment, the bsAb is a CrossMab with 3 F(ab) regions.

In other embodiments, the bsAb is tetravalent in a 2+2 format (i.e., 2 binding sites for each target). In a further embodiment, the bsAb is a fusion of a normal IgG with 2 scFv domains, Bs4Ab, DVD-Ig, tetravalent DART-Fc, four scFv domains fused to Fc, CODV-Ig, a pair of tandem VHH nanobodies fused to Fc, or a CrossMab with 4 F(ab) regions.

Examples of bsAbs that may be used as targeting polypeptides of the fusion proteins of the invention include the following: CD3×B7-H3 (e.g., orlotamab), CD3×BCMA (e.g., AMG420, AMG701, EM801, JNJ-64007957, PF-06863135, REGN5458), CD3×CD19 (e.g., A-319, AFM11, AMG562, blinatumomab), CD3×CD20 (e.g., mosunetuzumab, plamatomab, REGN1979, CD20-TCB), CD3×CD33 (e.g., AMG330, AMG673, AMV-564, GEM333), CD3×CD38 (e.g., AMG424, GBR1342), CD3×CEA (e.g., Cibisatamab), CD3×EGFRvIII (e.g., AMG596), CD3×EpCAM (e.g., A-337, catumaxomab, removab), CD3×FLT3 (e.g., AMG427), CD3×GPC3 (e.g., ERY974), CD3×gpA33 (e.g., MGD007), CD3×GPRC5D (e.g., JNJ-64407564), CD3×HER2 (e.g., GBR1302, M802, RG6194), CD3×MUC16 (e.g., REGN4018), CD3×P-Cadherin (e.g., PF-06671008), CD3×PSMA (e.g., AMG160, MOR209, pasotuxizumab), CD3×SSTR2 (e.g., tidutamab), CD40×MSLN (e.g., ABBV-428), PD-1×ICOS (e.g., Xmab23104), or PD-L1×4-1BB (e.g., MCLA-145)

Exemplary fusion proteins of the invention comprising one or more receptor ECDs and a bispecific antibody include anti-CEACAM5/CD3 (cibisatamab) fused to BTLA on HC—e.g., SEQ ID NOs: 747, 748, 218, 219; anti-CEACAM5/CD3 (cibisatamab) fused to BTLA on LC—e.g., SEQ ID NOs: 216, 217, 749, 219; anti-CEACAM5/CD3 (cibisatamab) fused to PD1 on HC—e.g., SEQ ID NOs: 751, 752, 218, 219; anti-CEACAM5/CD3 (cibisatamab) fused to PD1 on LC—e.g., SEQ ID NOs: 216, 217, 753, 219; anti-CEACAM5/CD3 (cibisatamab) fused to SIGLEC10 on HC—e.g., SEQ ID NOs: 755, 756, 218, 219; anti-CEACAM5/CD3 (cibisatamab) fused to SIGLEC10 on LC—e.g., SEQ ID NOs: 216, 217, 757, 219; anti-CEACAM5/CD3 (cibisatamab) fused to SIRPa on HC—e.g., SEQ ID NOs: 759, 760, 218, 219; anti-CEACAM5/CD3 (cibisatamab) fused to SIRPa on LC—e.g., SEQ ID NOs: 216, 217, 761, 219; anti-CEACAM5/CD3 (cibisatamab) fused to TGFbR on HC—e.g., SEQ ID NOs: 763, 764, 218, 219; anti-CEACAM5/CD3 (cibisatamab) fused to TIM3 on HC—e.g., SEQ ID NOs: 765, 766, 218, 219; anti-CEACAM5/CD3 (cibisatamab) fused to TIM3 on LC—e.g., SEQ ID NOs: 216, 217, 767, 219; anti-CEACAM5/CD3 (cibisatamab) fused to VEGFR on HC—e.g., SEQ ID NOs: 769, 770, 218, 219; anti-CD3/PSMA (pasotuxizumab) fused to BTLA—e.g., SEQ ID NO: 729; anti-CD3/PSMA (pasotuxizumab) fused to PD1—e.g., SEQ ID NO: 733; anti-CD3/PSMA (pasotuxizumab) fused to SIGLEC10—e.g., SEQ ID NO: 737; anti-CD3/PSMA (pasotuxizumab) fused to SIRPa—e.g., SEQ ID NO: 741; anti-CD3/PSMA (pasotuxizumab) fused to TIM3—e.g., SEQ ID NO: 745.

In some embodiments, one of the targets of the bispecific antibody targeting polypeptide is CD28.

In some embodiments, this targeting polypeptide is an antibody. In such cases, the fusion protein comprising an antibody and one or more ECDs may be referred to as an “antibody-ligand trap”, or “ALT”, which are used interchangeably.

In one embodiment, the targeting polypeptidein is an immunoglobulin. As used herein, the term “immunoglobulin” includes natural or artificial mono- or polyvalent antibodies including, but not limited to, polyclonal, monoclonal, multispecific, human, humanized or chimeric antibodies, single chain antibodies, Fab fragments. F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin ion can be of any type (e.g., IgG, IgE, IgM, IgD, IgA, and IgY), class (e.g., IgG1, IgG2, IgG3, IgG4, IgA1, and IgA2) or subclass of immunoglobulin molecule.

An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In one embodiment, the targeting moiety is an antibody or scFv.

An antigen-binding antibody fragment, including single-chain antibody, may include the variable region(s) alone or in combination with the entirety or a portion of the following: hinge region, CH1, CH2, and CH3 domains. An antigen-binding fragment can also include any combination of variable region(s) with a hinge region, CHI, CH2, and CH3 domains. Also includes is a Fc fragment, antigen-Fc fusion proteins, and Fc-targeting moiety. The antibody may be from any animal origin including birds and mammals. In one aspect, the antibody is, or derived from, a human, murine (e.g., mouse and rat), donkey, sheep, rabbit, goat, guinea pig, camel, horse, or chicken. Further, such antibody may be a humanized version of an antibody. The antibody may be monospecific, bispecific, trispecific, or of greater multi specificity.

The intact antibody may have one or more “effector functions” which refer to those biological activities attributable to the Fc region (a native sequence Fc region or amino acid sequence variant Fc region or any other modified Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells. In one embodiment, the targeting antibody or Fc-containing fusion protein facilitates focused or preferential delivery of a immunomodulatory moiety to a target cell. In another aspect, a targeting antibody can induce death of the targeted cell or sensitize it to immune cell-mediated cytotoxicity. In another aspect, the Fc-fusion protein or antibody can facilitate delivery of the immunomodulatory moiety or immunogenic apoptotic material from antibody-bound tumor targets, or both, to an antigen presenting cells (APC) via interactions between their Fc and Fc receptors (on APC).

The Fc region may have one or more modifications to alter one or more of its biophysical and/or functional properties; for example, extend half-life, reduce effector function, or increase effector function. Such modifications are well-known in the art. Exemplary modifications include the “LALA” (L234A/L235A) mutation of IgG1, and the S228P mutation of IgG4.

The term “fragment” refers to any subject polypeptide having an amino acid residue sequence shorter than that of a polypeptide whose amino acid residue sequence is disclosed herein

In some embodiments, this targeting polypeptide binds a tumor-associated antigen or tumor antigen. In one embodiment, a “tumor-associated antigen” is a molecule whose expression is elevated on tumor cells. In one embodiment, the tumor-associated antigen is a growth factor receptor or a growth factor.

In one embodiment, the fusion protein of the invention comprises a targeting polypeptide that specifically binds a tumor-associated molecule. In some embodiments, the targeting polypeptide binds one of the following targets: CA125, CA19-9, CD30, CEACAM5, CEACAM1, CEACAM6, DLL3, DLL4, DPEP3, EGFR, EGFRvIII, GD2, HER2, HER3, HGF, IGF1R, IL13Ra2, LIV-1, LRRC15, MUC1, PRLR, PSCA, PSMA, PTK7, SEZ6, SLAMF7, TF, cMet, claudin, mesothelin, nectin4, uPAR, GPNMB, CD79b, CD22, NaPi2b, SLTRK6, STEAP1, MUC16, CD37, GCC, AGC-16, 5T4, CD70, TROP2, CD74, CD27L, Fra, CD138, CA6.

In various embodiments, the fusion protein of the invention comprises an antibody selected from the following: 41BB: urelumab (SEQ ID NOs: 1, 2); CA125: ab1 (SEQ ID NOs: 3, 4), sofituzumab (SEQ ID NOs: 5, 6); CA19-9: MVT-5873 (SEQ ID NOs: 7, 8); CD20: rituximab (SEQ ID NOs: 9, 10); CD30: brentuximab (SEQ ID NOs: 11, 12); CD33: gemtuzumab (SEQ ID NOs: 13, 14); CD38: daratumumab (SEQ ID NOs: 15, 16); CD39: IPH5201 (SEQ ID NOs: 17, 18); CD40: ABBV-428 (SEQ ID NOs: 19, 20); CD47: 5F9 (SEQ ID NOs: 21, 22); CD73: GS1423 (SEQ ID NOs: 23, 24); CEACAM5: labetuzumab (SEQ ID NOs: 25, 26); CTLA4: ipilimumab (SEQ ID NOs: 27, 28); DLL3: rovalpituzumab (SEQ ID NOs: 29, 30); DLL4: ABT-165 (SEQ ID NOs: 31, 32); DPEP3: ab1 (SEQ ID NOs: 33, 34), ab2 (SEQ ID NOs: 35, 34); EGFR: panitumumab (SEQ ID NOs: 36, 37), necitumumab (SEQ ID NOs: 38, 39), ABBV-321 (SEQ ID NOs: 40, 41), cetuximab (SEQ ID NOs: 42, 43); EGFRvIII: ab1 (SEQ ID NOs: 44, 45), depatuxizumab (SEQ ID NOs: 46, 47); GARP: ARGX-115 (SEQ ID NOs: 48, 49); GD2: dinutuximab (SEQ ID NOs: 50, 51); HER2: pertuzumab (SEQ ID NOs: 52, 53), trastuzumab (SEQ ID NOs: 54, 55); HGF: rilotumumab (SEQ ID NOs: 56, 57); ICOS: GSK3359609 (SEQ ID NOs: 58, 59); IL13Ra2: ab1 (SEQ ID NOs: 60, 61); IL17R: brodalumab (SEQ ID NOs: 62, 63); ILla: 3D12r16 (SEQ ID NOs: 64, 65); ILlb: E26.35 (SEQ ID NOs: 66, 67), canakinumab (SEQ ID NOs: 68, 69); IL23: brazikumab (SEQ ID NOs: 70, 71), guselkumab (SEQ ID NOs: 72, 73), risankizumab (SEQ ID NOs: 74, 75), tildrakizumab (SEQ ID NOs: 76, 77); IL6R: tocilizumab (SEQ ID NOs: 78, 79); LAP: 7H4 (SEQ ID NOs: 80, 81); LIV-1: ladiratuzumab (SEQ ID NOs: 82, 83); LRRC15: ABBV-085-huAD208.4.1 (SEQ ID NOs: 84, 85), ABBV-085-huM25 (SEQ ID NOs: 86, 87); MUC1: clivatuzumab (SEQ ID NOs: 88, 89); OX40: ABBV-368-Hu3726 (SEQ ID NOs: 90, 91), ABBV-368-Hu3739 (SEQ ID NOs: 92, 93), ABBV-368-Hu3741 (SEQ ID NOs: 94, 95), GSK3174998 (SEQ ID NOs: 96, 97); PD1: spartalizumab (SEQ ID NOs: 98, 99), pembrolizumab (SEQ ID NOs: 100, 101), ABBV-181 (SEQ ID NOs: 102, 103), nivolumab (SEQ ID NOs: 104, 105); PDGF: ab1 (SEQ ID NOs: 106, 107); PDL1: atezolizumab (SEQ ID NOs: 108, 109), avelumab (SEQ ID NOs: 110, 111), durvalumab (SEQ ID NOs: 112, 113); PRLR: ab1 (SEQ ID NOs: 114, 115); PSCA: ab1 (SEQ ID NOs: 116, 117); PSMA: ab2 (SEQ ID NOs: 118, 119), ab1 (SEQ ID NOs: 120, 121); PTK7: hSC6.24 (SEQ ID NOs: 122, 123); SEZ6: hSC17.200 (SEQ ID NOs: 124, 125); SLAMF7: elotuzumab (SEQ ID NOs: 126, 127); TF: tisotumab (SEQ ID NOs: 128, 129); TGFb: XOMA-089 (SEQ ID NOs: 130, 131), fresolimumab (SEQ ID NOs: 132, 133); TGFbRII: LY3022859 (SEQ ID NOs: 134, 135); TIGIT: etigilimab (SEQ ID NOs: 136, 137), tiragolumab (SEQ ID NOs: 138, 139); TIM3: M6903 (SEQ ID NOs: 140, 141); VEGF: ranibizumab (SEQ ID NOs: 142, 143), bevacizumab (SEQ ID NOs: 144, 32), ABT-165 (SEQ ID NOs: 145, 146), ab1 (SEQ ID NOs: 144, 32); VEGFR: ramucirumab (SEQ ID NOs: 147, 148); VISTA: onvatilimab (SEQ ID NOs: 149, 150); cMet: telisotuzumab (SEQ ID NOs: 151, 152); claudin: hSC27.1 (SEQ ID NOs: 153, 154); gp120: B12 (SEQ ID NOs: 155, 156); mesothelin: ABBV-428 (SEQ ID NOs: 157, 158); nectin4: enfortumab (SEQ ID NOs: 159, 160); uPAR: ab1 (SEQ ID NOs: 161, 162), ab2 (SEQ ID NOs: 163, 164).

In one embodiment, the targeting polypeptide specifically binds human epidermal growth factor receptor 2 (HER2; ErbB2). HER2 is overexpressed in many human cancers, including breast cancer and gastric cancer. In HER2-overexpressing cells, excess levels of HER2 expression can result in spontaneous and constitutive ligand-independent dimerization with subsequent activation of the cytoplasmic kinase region. HER2 can additionally heterodimerize with HER3 and EGFR. Each of these interactions leads to kinase signaling that stimulates phosphorylation and downstream signaling, primarily through the PI3K-Akt-mTOR and Ras-Raf-MEK-Erk pathways. Activation of these pathways promotes proliferation, evasion of apoptosis, angiogenesis, and invasion, leading to tumor growth and progression. anti-HER2 antibody (trastuzumab) inhibits homodimerization and autophosphorylation of HER2, as well as heterodimerization of HER2 with EGFR. anti-HER2 antibody (pertuzumab) interrupts the HER2/HER3 interaction or downstream signaling of the HER2/HER3 heterodimer in complex with HER3 ligand (heregulin). In some embodiments, the antibody of the ALT that binds HER2 is selected from one of the following: pertuzumab (SEQ ID NOS. 52, 53); trastuzumab (SEQ ID NOS. 54, 55). In one aspect, the HER2 targeted antibody is conjugated to a cytotoxic agent (anti-HER2-ADC), such as ado-trastuzumab (Trastuzumab-DM1). In various embodiments, the fusion protein of the invention comprises an antibody that targets and inhibits HER2 fused to one or more receptor ECDs.

In one embodiment, the targeting polypeptide specifically binds epidermal growth factor receptor (EGFR). In epithelial tumors, the epidermal growth factor receptor (EGFR) controls key signaling pathways responsible for growth, proliferation, migration, and survival of tumor cells. The epidermal growth factor receptor variant III (EGFRvIII) is the most common EGFR mutation that occurs frequently in high-grade gliomas especially glioblastoma multiforme (GBM). EGFRvIII arises from the deletion of exon 2-7 that leads to the formation of the constitutively activated mutant receptor incapable of binding any known ligand. EGFRvIII-expressing cells are resistant to EGFR inhibitors, and EGFRvIII expression in tumors is often correlates with poor prognosis. The presence of the unique glycine site in EGFRvIII provides an option to develop EGFRvIII-specific monoclonal antibodies. Examples of antibodies targeting EGFRvIII include depatuxizumab. Examples of EGFR antibodies include cetuximab, panitumumab, and necitumumab. In some embodiments, the antibody of the ALT that binds EGFR is selected from one of the following: panitumumab (SEQ ID NOS. 36, 37); necitumumab (SEQ ID NOS. 38, 39); ABBV-321 (SEQ ID NOS. 40, 41); cetuximab (SEQ ID NOS. 42, 43). In various embodiments, the fusion protein of the invention comprises a polypeptide that targets and inhibits EGFR or EGFRvIII fused to one or more receptor ECDs.

In one embodiment, the targeting polypeptide specifically binds Prostate-specifc membrane antigen (PSMA). PSMA is a non-soluble type 2 integral membrane protein. It is weakly expressed in normal prostate tissue but strongly upregulated in prostate cancer. It is also expressed in the neovasculature of numerous solid malignancies. PSMA overexpression is associated with higher Prostate Cancer grade and androgen deprivation, further increasing in metastatic disease and castration resistant Prostate Cancer. In one embodiment, the ALT specifically binds an epitope of PSMA necessary for its functional activity, such as PI3K activation. In some embodiments, the antibody of the ALT that binds PSMA is selected from one of the following: ab2 (SEQ ID NOS. 118, 119); ab1 (SEQ ID NOS. 120, 121). In one aspect, the PSMA targeted antibody is conjugated to a cytotoxic agent (anti-PSMA-ADC). In various embodiments, the fusion protein of the invention comprises a polypeptide that targets and binds PSMA fused to one or more receptor ECDs.

In one embodiment, the targeting polypeptide specifically binds the urokinase-type plasminogen activator receptor (uPAR). uPAR is a GPI-anchored cell membrane receptor, composed by three homologous domains (DI, DII, DIII). Its main function is focusing of urokinase (uPA) proteolytic activity, responsible for degradation of extracellular matrix (ECM) components, on the cell surface. When uPA binds uPAR, it consequently converts plasminogen to active plasmin, which activates several proteases related to the degradation of extracellular matrix proteins and basal membranes, thereby facilitating cancer cell invasion and metastasis. uPAR expression is increased in many human cancers and correlates with a poor prognosis and early invasion and metastasis. uPAR is an adhesion receptor, as it binds vitronectin (VN), an abundant component of provisional extracellular matrix (ECM). This direct interaction between uPAR and VN is critical for triggering changes in cell morphology, migration and signaling and is an important requirement for the induction of epithelial mesenchymal transition (EMT). In some embodiments, the antibody of the ALT specifically inhibits the uPA/uPAR interaction. In other embodiments, the ALT specifically inhibits the vitronectin/uPAR interaction. In one aspect the antibody binds a sequence or domain of uPAR that remains on the cell surface following cleavage. In some embodiments, the antibody of the ALT that binds uPAR is selected from one of the following: ab1 (SEQ ID NOS. 161, 162); ab2 (SEQ ID NOS. 163, 164). In one aspect, the uPAR targeted antibody is conjugated to a cytotoxic agent (anti-uPAR-ADC). In various embodiments, the fusion protein of the invention comprises a polypeptide that targets and binds uPAR, fused to one or more receptor ECDs.

In some embodiments, the targeting polypeptide binds an antigen overexpressed by a hematologic malignancy. In some embodiments, the targeting polypeptide binds an antigen overexpressed by multiple myeloma. In some embodiments, the targeting polypeptide binds CD38, SLAMF7, or BCMA. In some embodiments, the targeting polypeptide is an antibody selected from the following list: MEDI2228; CC-99712; belantamab; Gemtuzumab (anti-CD33 mAb). In some embodiments, the targeting polypeptide binds an antigen overexpressed by Non-Hodgkin's B cell lymphomas. In some embodiments, the antibody binds CD20. In some embodiments, the targeting polypeptide binds rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb). In some embodiments, the targeting polypeptide binds CD19. In some embodiments, the targeting polypeptide binds an antigen overexpressed by Hodgkin's lymphomas. In some embodiments, the antibody binds CD30, or CD22. In some embodiments, the targeting polypeptide binds an antigen overexpressed by leukemia. In some embodiments, the ALT binds CD33.

T cell activation begins with the recognition of an antigenic peptide in the context of a major histocompatibility complex (MHC) on an antigen-presenting cell by the T cell receptor (TCR). The process of T cell activation is mediated by a number of signaling proteins through inducible phosphorylation, enzyme activation and protein-protein and protein-lipid interactions. T cells require two signals to become fully activated. A first signal, which is antigen-specific, is provided through the T cell receptor (TCR) which interacts with peptide-MHC molecules on the membrane of antigen presenting cells (APC). A second signal, the co-stimulatory signal, is antigen nonspecific and is provided by the interaction between co-stimulatory molecules expressed on the membrane of APC and the T cell. T cell co-stimulation is necessary for T cell proliferation, differentiation and survival. Activation of T cells without co-stimulation may lead to T cell anergy, T cell deletion or the development of immune tolerance.

The best characterized T cell co-stimulatory molecules expressed by T cells is CD28 which interacts with CD80 (B7.1) and CD86 (B7.2) on the membrane of APC. Other costimulatory receptors expressed by T cells include 4-1BB (receptor for 4-1BB ligand), ICOS (Inducible Costimulator) (receptor for ICOS-L), OX40 (receptor for OX40 ligand), GITR (receptor for GITR ligand), CD27 (interacts with CD70), CD40L/CD40, HVEM (interacts with LIGHT), CD226 (interacts with CD155).

The activation signals are modulated by a family of receptors termed, T cell co-inhibitory receptors that include CTLA-4, PD-1, LAG-3, TIM-3, CEACAM-1, TIGIT, CD96, BTLA, CD160, VISTA, VSIG8, LAIR. Co-inhibitory receptors modulate signaling by utilizing mechanisms such as ectodomain competition with counter receptors and by the use of intracellular mediators such as protein phosphatases. Co-inhibitory receptors can act as threshold-setters, modulators, checkpoints and feedback mechanisms that can fine tune the quality and magnitude of the T cell immune response.

Receptors that are inhibitory to T cell function are termed T cell co-inhibitory receptors. Inhibitory receptors attenuate and counterbalance activation signals initiated by stimulatory receptors. The subsequent outcomes on T cell function can range from temporary inhibition to permanent inactivation and cell death. TCR signaling can be controlled by various mechanisms that differ in their time of action and/or target molecule. Negative regulatory mechanisms are in place to act before T cell activation to maintain its quiescent state.

The majority of T cell co-inhibitory receptors belong to the immunoglobulin (Ig) superfamily. One mechanism involves the sequestration of the ligands for co-stimulatory receptors, depriving the T cell from receiving activation signals necessary for complete activation. The second mechanism involves the recruitment of intracellular phosphatases by an immunoreceptor tyrosine-based inhibition motif (ITIM) and/or an immunoreceptor tyrosine-based switch motif (ITSM) that make up the cytoplasmic tail of certain inhibitory receptors, which dephosphorylate signalling molecules downstream of the TCR and co-stimulatory pathways, leading to a quantitative reduction in activation-induced gene expression. The third mechanism involves the upregulation (or downregulation) of genes that code for proteins involved in the inhibition of immune functions. A co-inhibitory receptor could use a combination of the above and possibly other yet to be discovered mechanisms to regulate T cell signaling.

T cell co-inhibitory receptors are transmembrane glycoproteins that transmit dominant negative signals mainly via intracellular phosphatases that bind to phosphorylated tyrosine residues in the cytoplasmic domain. T cell co-inhibitory receptors can act as safety mechanisms and threshold setters to prevent uncontrolled detrimental extremes of reactivity by counteracting the stimulatory signals.

In one embodiment, the fusion protein of the invention comprises a targeting polypeptide that specifically binds a T cell co-inhibitory receptor (TCIR), a T cell co-inhibitory receptor ligand (TCIR ligand), a T-cell co-inhibitory molecule, or a T cell co-stimulatory molecule. In an additional aspect, the antibody is an antagonist of a TCIR, TCIR ligand, or T cell co-inhibitory molecule. In an additional aspect, the targeting moiety polypeptide specifically binds one or more of the following molecules: Cytotoxic T lymphocyte associated antigen-4 (CTLA-4, CD152), Programmed Death-1 protein (PD-1), Programmed death ligand-1 (PD-L1), Programmed death ligand (PD-L2), B7-H3 (CD276), T-cell immunoglobulin and mucin-domain containing-3 (TIM-3), Carcinoembryonic antigen-related cell adhesion molecule (CEACAM), V domain Ig suppressor of T cell activation (VISTA), V-set and immunoglobulin domain containing 8 (VSIG8), B and T lymphocyte attenuator (BTLA), Herpesvirus Entry Mediator (HVEM), CD160, T cell Ig and ITIM domain (TIGIT), CD226, CD96, Lymphocyte activation gene-3 (LAG-3).

In another aspect, the targeting polypeptide is an agonist of a T cell co-stimulatory molecule. In one aspect, the targeting polypeptide is an antibody that binds a T cell co-stimulatory molecule as an agonist. In another aspect, the targeting polypeptide is the extracellular domain of a native agonist ligand of a T cell co-stimulatory molecule. In an additional aspect, the targeting polypeptide specifically binds one of the following molecules: 4-1BB (CD137), Inducible T-Cell Costimulator (ICOS), OX-40 (CD134), glucocorticoid-induced TNFR-related protein (GITR), CD40, DNAM, CD30, or CD27. In various embodiments, an ALT of the invention comprises an antibody that binds a T cell co-inhibitory molecule or a T cell co-stimulatory molecule, wherein the said antibody is fused with one or more receptor ECDs.

In one embodiment, the fusion protein of the invention comprises a targeting polypeptide that specifically binds a “don't eat me” or anti-phagocytic ligand or receptor that inhibits the function of macrophages, dendritic cells, or other innate immune cells. Anti-phagocytic ligands expressed by cells bind their cognate receptor on a macrophage, dendritic cell, or other innate immune cell to inhibit phagocytosis. Tumor cells take advantage of this anti-phagocytic mechanism and overexpress “don't eat me” ligands in order to inhibit innate immune cell antitumor activity. In some embodiments, the targeting polypeptide binds CD47, SIRPa, CD31, CD24, SIGLEC10, or LILRB1.

In another aspect, the targeting polypeptide binds and disables the interaction of CD47 and SIRPa. CD47 is a “don't eat me signal” expressed by tumor cells that binds SIRPa on macrophages and induces SHP1/SHP2-mediated inhibition of macrophage phagocytosis. In various embodiments, an ALT of the invention comprises an antibody that binds and disables CD47, wherein the said antibody is fused with one or more receptor ECDs. In another aspect, the targeting polypeptide binds and disables a different “don't eat me” interaction—for example, LILRB1/MHC, SIGLEC10/CD24, or CD31/CD31. In various embodiments, the targeting polypeptide is an antibody that binds one of these targets.

In one embodiment, the targeting polypeptide comprises a polypeptide or antibody that specifically binds Programmed death-1 (PD-1; CD279) or Programmed death-1 ligands [PD-L1 (B7-H1); PD-L2 (B7-H4)]. Tumor cells express PD-1 ligands which inhibit T cell effector function and induce T cell exhaustion/apoptosis via engagement of PD-1. In one embodiment, the ALT comprises an antibody that specifically binds PD-1 and disrupts its interaction with PD-L1 or PD-L2. In one embodiment, the ALT comprises an antibody that specifically binds PD-L1 and disrupts its interaction with either PD-1 or B7: atezolizumab (SEQ ID NOS. 108, 109); avelumab (SEQ ID NOS. 110, 111); durvalumab (SEQ ID NOS. 112, 113). In another embodiment, the ALT comprises an antibody that specifically binds PD-1: spartalizumab (SEQ ID NOS. 98, 99); pembrolizumab (SEQ ID NOS. 100, 101); ABBV-181 (SEQ ID NOS. 102, 103); nivolumab (SEQ ID NOS. 104, 105).

In one embodiment, the invention comprises fusion proteins comprising targeting polypeptides wherein the targeting polypeptide is an antibody fused to one or more ECDs. In one aspect, the targeting polypeptide is an antibody-drug conjugate (ADC). In one aspect, the antibody is conjugated to one or more cytotoxic agents. In some embodiments, the cytotoxic agent causes immunogenic cell death. In some embodiments, the cytotoxic agent causes genotoxic cell death.

In some embodiments, the drug conjugate is selected from: mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

The cytotoxic agent conjugated to the targeting polypeptide antibody may be any agent that induces cell death. In various embodiments, the cytotoxic agent may be selected from, but is not limited to, the following list: (1) maytansinoid (DM1), (2) calcheamicin, (3) auristatin (e.g., monomethyl auristatin E (MMAE) or monomethyl auristatin F (MMAF)).

In some embodiments, the cytotoxic agent may be conjugated to cysteines. In other embodiments, the cytotoxic agent may be conjugated to lysines. In some embodiments, the cytotoxic agent may be conjugated via a cleavable linker. In some embodiments, the cytotoxic agent may be conjugated via a non-cleavable linker. In various embodiments, the cytotoxic agent may be linked to the targeting polypeptide antibody via a linker, which may be selected from, but is not limited to, the following list: (1) hydrazone, (2) SMCC (maleimide), (3) valine-citrulline, (4) 4AP, (5) maleimidocaproyl (mc), (6) maleimidomethyl cyclohexane-1-carboxylate (mcc). The linker may further comprise one or more spacers. In some embodiments, the spacer may be selected from thiol-reactive maleimidocaproyl spacer and p-amino-benzyloxycarbonyl spacer. In one embodiment, the cleavable linker is maleimidocaproyl-valyl-citrullinyl-p-aminobenzyloxycarbonyl (mc-val-cit-PABC).

In one embodiment, a tumor-targeted antibody is fused to one or more receptor extracellular domains and conjugated to one or more cytotoxic agents. In one embodiment, a fusion protein comprises a targeting polypeptide wherein the targeting polypeptide is an antibody-drug conjugate and the targeting polypeptide is fused to a ligand-binding sequence of a receptor ECD. In one aspect, the receptor ECD is fused to the heavy chain of the targeting polypeptide. In another aspect, the receptor ECD is fused to the light chain of the targeting polypeptide.

In one embodiment, a fusion protein comprises a targeting polypeptide wherein the targeting polypeptide is an antibody-drug conjugate and the targeting polypeptide is fused to a ligand-binding sequence of TGFbRII ECD, or a fragment thereof. In one embodiment, a fusion protein comprises a targeting polypeptide wherein the targeting polypeptide is an antibody-drug conjugate and the targeting polypeptide is fused to a ligand-binding sequence of PD1 ECD, or a fragment thereof. In one embodiment, a fusion protein comprises a targeting polypeptide wherein the targeting polypeptide is an antibody-drug conjugate and the targeting polypeptide is fused to a ligand-binding sequence of BTLA ECD, or a fragment thereof. In one embodiment, a fusion protein comprises a targeting polypeptide wherein the targeting polypeptide is an antibody-drug conjugate and the targeting polypeptide is fused to a ligand-binding sequence of TIM-3 ECD, or a fragment thereof. In one embodiment, a fusion protein comprises a targeting polypeptide wherein the targeting polypeptide is an antibody-drug conjugate and the targeting polypeptide is fused to a ligand-binding sequence of SIRPa ECD, or a fragment thereof. In one embodiment, a fusion protein comprises a targeting polypeptide wherein the targeting polypeptide is an antibody-drug conjugate and the targeting polypeptide is fused to a ligand-binding sequence of SIGLEC10 ECD, or a fragment thereof. In one embodiment, a fusion protein comprises a targeting polypeptide wherein the targeting polypeptide is an antibody-drug conjugate and the targeting polypeptide is fused to a ligand-binding sequence of VEGFR ECD, or a fragment thereof.

In various embodiments, the targeting polypeptide is an antibody-drug conjugate selected from: gemtuzumab ozogamicin, brentuximab vedotin, trastuzumab emtansine, inotuzumab ozogamicin, polatuzumab vedotin, enfortumab vedotin, trastuzumab deruxtecan, or sacituzumab govitecan.

In one embodiment, the fusion protein comprises anti-nectin-4 antibody fused to TGFbRII on the C-terminus of the heavy chain; and MMAE is conjugated to the antibody via a protease cleavable linker comprising maleimidocaproyl, valine-citrulline, and PABC. In one aspect, this fusion protein is anti-nectin4-TGFbRII (e.g., SEQ ID NOs: 265, 160). In one embodiment, the fusion protein comprises anti-nectin-4 antibody fused to BTLA on the C-terminus of the heavy chain; and MMAE is conjugated to the antibody via a protease cleavable linker comprising maleimidocaproyl, valine-citrulline, and PABC. In one aspect, this fusion protein is anti-nectin4-BTLA (e.g., SEQ ID NOs: 256, 160). In one embodiment, the fusion protein comprises anti-nectin-4 antibody fused to SIRPa on the C-terminus of the heavy chain; and MMAE is conjugated to the antibody via a protease cleavable linker comprising maleimidocaproyl, valine-citrulline, and PABC. In one aspect, this fusion protein is anti-nectin4-SIRPa (e.g., SEQ ID NOs: 264, 160). In one embodiment, the fusion protein comprises anti-nectin-4 antibody fused to PD1 on the C-terminus of the heavy chain; and MMAE is conjugated to the antibody via a protease cleavable linker comprising maleimidocaproyl, valine-citrulline, and PABC. In one aspect, this fusion protein is anti-nectin4-PD1 (e.g., SEQ ID NOs: 261, 160). In one embodiment, the fusion protein comprises anti-nectin-4 antibody fused to TIM3 on the C-terminus of the heavy chain; and MMAE is conjugated to the antibody via a protease cleavable linker comprising maleimidocaproyl, valine-citrulline, and PABC. In one aspect, this fusion protein is anti-nectin4-TIM3 (e.g., SEQ ID NOs: 266, 160). In one embodiment, the fusion protein comprises anti-nectin-4 antibody fused to SIGLEC10 on the C-terminus of the heavy chain; and MMAE is conjugated to the antibody via a protease cleavable linker comprising maleimidocaproyl, valine-citrulline, and PABC. In one aspect, this fusion protein is anti-nectin4-SIGLEC10 (e.g., SEQ ID NOs: 263, 160).

In one embodiment, the fusion protein comprises anti-HER2 antibody fused to TGFbRII on the C-terminus of the heavy chain; and DM1 is conjugated to the antibody via a linker comprising mcc. In one aspect, this fusion protein is anti-HER2-TGFbRII (e.g., SEQ ID NOs: 253, 55). In one embodiment, the fusion protein comprises anti-HER2 antibody fused to BTLA on the C-terminus of the heavy chain; and DM1 is conjugated to the antibody via a linker comprising mcc. In one aspect, this fusion protein is anti-HER2-BTLA (e.g., SEQ ID NOs: 244, 55). In one embodiment, the fusion protein comprises anti-HER2 antibody fused to TIM-3 on the C-terminus of the heavy chain; and DM1 is conjugated to the antibody via a linker comprising mcc. In one aspect, this fusion protein is anti-HER2-TIM3 (e.g., SEQ ID NOs: 254, 55). In one embodiment, the fusion protein comprises anti-HER2 antibody fused to PD1 on the C-terminus of the heavy chain; and DM1 is conjugated to the antibody via a linker comprising mcc. In one aspect, this fusion protein is anti-HER2-PD1 (e.g., SEQ ID NOs: 249, 55). In one embodiment, the fusion protein comprises anti-HER2 antibody fused to SIRPa on the C-terminus of the heavy chain; and DM1 is conjugated to the antibody via a linker comprising mcc. In one aspect, this fusion protein is anti-HER2-SIRPa (e.g., SEQ ID NOs: 252, 55). In one embodiment, the fusion protein comprises anti-HER2 antibody fused to SIGLEC10 on the C-terminus of the heavy chain; and DM1 is conjugated to the antibody via a linker comprising mcc. In one aspect, this fusion protein is anti-HER2-SIGLEC10 (e.g., SEQ ID NOs: 251, 55). In one embodiment, the fusion protein comprises anti-HER2 antibody fused to VEGFR on the C-terminus of the heavy chain; and DM1 is conjugated to the antibody via a linker comprising mcc. In one aspect, this fusion protein is anti-HER2-VEGFR (e.g., SEQ ID NOs: 255, 55).

In one embodiment, the fusion protein comprising a tumor-targeted antibody and one or more ECDs is expressed using recombinant methods well-known in the art, and then a conjugation procedure well-known in the art is applied to attach the cytotoxic agent to the fusion protein. In some embodiments, the cytotoxic agent may be conjugated to the fusion protein using cysteine-specific conjugation methods well-known in the art. In other embodiments, the cytotoxic agent may be conjugated to the fusion protein using lysine-specific conjugation methods well-known in the art. In other embodiments, the cytotoxic agent may be conjugated to the fusion protein in a site-specific manner well-known in the art. In some embodiments, this may be achieved via HIPS ligation (Hydrazinyl-Iso-Pictet-Spengler (HIPS) ligation to formylglycine), trapped Knoevenagel condensation, or tandem Knoevenagel condensation-Michael Addition (TKM) ligation.

In various embodiments, the fusion proteins of the invention counteract VEGF in the tumor microenvironment. In some embodiments, the fusion proteins comprise a ligand-binding sequence of an extracellular domain of VEGFR (e.g., VEGFR ECD).

In one aspect, the fusion proteins of the invention comprise an antibody that targets a tumor antigen or tumor-associated antigen expressed in the TME, wherein said antibody is fused to a VEGF-binding sequence from one or more extracellular domains of VEGFR (e.g. VEGFR1ECD and/or VEGFR2ECD). In one example, the ALT comprises vascular endothelial growth factor (VEGF) binding portions from the extracellular domains of human VEGF receptors 1 and 2. The VEGFR ECD fused to the ALT localizes to the TME, where it serves as a decoy receptor to bind and disable VEGF (e.g VEGF-A, VEGF-B). In another aspect an ALT comprising a fused ligand-binding sequence of VEGFR ECD is additionally fused to a different receptor ECD that captures and disables its cognate ligands (e.g. TGFbRII ECD, PD-1ECD, TIM-3ECD, SIRPa ECD, BTLA ECD). In one aspect, the VEGFR ECD is fused to the C-terminus of the heavy chain of the targeting antibody, and another different receptor ECD is fused to the C-terminus of the light chain.

In other embodiments, the fusion proteins of the invention comprise a targeting polypeptide that binds VEGF or VEGFR fused to one or more receptor ECDs. These receptor ECDs are preferably selected from the following: PD1 ECD, TIM-3 ECD, TGFbRII ECD, BTLA ECD, SIRPa ECD, SIGLEC10 ECD. In some embodiments, the targeting polypeptide that binds VEGF or VEGFR is an antibody.

In another aspect, the ALT is a polypeptide comprising an antibody that targets VEGF or VEGFR, wherein said antibody is fused to a ligand-binding sequence of an extracellular domain of a receptor. In one example, the ALT comprises an antibody that targets either VEGF or VEGFR, wherein said antibody is fused to a TGFb-binding sequence of an extracellular domain of the TGFbR (e.g. TGFbRII ECD). In another example, the ALT comprises an antibody that targets either VEGF or VEGFR, wherein said antibody is fused to a PD-1 ligand-binding sequence of an extracellular domain of PD-1 (PD-1ECD). In another example, the ALT comprises an antibody that targets either VEGF or VEGFR, wherein said antibody is fused to a TIM-3 ligand-binding sequence of an extracellular domain of TIM-3 (TIM-3ECD). In another example, the ALT comprises an antibody that targets either VEGF or VEGFR, wherein said antibody is fused to a CD47-binding sequence of an extracellular domain of SIRPa (e.g. SIRPa ECD). In another example, the ALT comprises an antibody that targets either VEGF or VEGFR, wherein said antibody is fused to a HVEM-binding sequence of an extracellular domain of BTLA (e.g. BTLA ECD). In another aspect an ALT comprises an antibody that targets either VEGF or VEGFR, wherein said antibody is fused to a ligand-binding sequence of a specific receptor ECD (receptor ECD-1), and additionally fused to a ligand-binding sequence of another different receptor ECD (receptor ECD-2). In various examples, receptor ECD-1 and receptor ECD-2 may be selected from a group comprising TGFbRII ECD, PD-1ECD, TIM-3ECD, BTLA ECD and SIRPa ECD. In one aspect, the receptor ECD-1 sequence is fused to the C-terminus of the heavy chain of the targeting antibody, and another different receptor ECD-2 sequence is fused to the C-terminus of the light chain. In one aspect, the receptor ECD (that is fused to a VEGF or VEGFR1 targeting antibody) binds to a cognate ligand expressed in the TME, thereby localizing the ALT and consequent VEGF/VEGFR blockade to the TME. In one example, a PD-1ECD binds PD-L1 or PD-L2 expressed on tumor cells or the TME. In another example, a TIM-3ECD binds CEACAM-1 or CEACAM-5 or CEACAM-6 expressed on tumor cells or the TME. In another example, a SIRPa ECD binds CD47 expressed on tumor cells or the TME. In another example, a BTLA ECD binds HVEM expressed on tumor cells or the TME.

Examples of such ALTs that capture and disable VEGF or block VEGFR signaling in the TME include, but are not limited to the following: ALTs comprising an antibody fused to VEGFR ECD (with or without another receptor ECD fused to the same antibody); ALTs where the antibody binds VEGF, fused to one or more Receptor ECD(s); ALTs where the antibody binds VEGFR, fused to one or more Receptor ECD(s)

In one embodiment, the fusion proteins of the invention may comprise a ligand-binding sequence of an extracellular domain of Vascular endothelial growth factor receptor (VEGFR1 and/or VEGFR2) to bind and disable VEGF.

In some embodiments, fusion proteins of the invention comprise VEGFR ECD and a polypeptide that inhibits CD47/SIRPa. Although CD47 targeted antibodies can promote antitumor immune responses by inhibiting the interaction of CD47 with SIRPa, its antitumor efficacy may be limited by disruption of TSP-1/CD47-dependent inhibition of VEGF and angiogenesis. In one embodiment, the ALT is a polypeptide comprising an antibody that targets CD47, wherein said antibody is fused to a VEGFR ECD. In this aspect, the ALT promotes antitumor immunity by disrupting the interaction of CD47 with SIRPa, while simultaneously counteracting VEGF-mediated tumor angiogenesis. In another aspect, the ALT comprises a VEGF-binding sequence from VEGFR ECD and a CD47-binding sequence from one or more extracellular domains of SIRPa (SIRPa ECD).

In some embodiments, the fusion protein comprises VEGFR ECD and a polypeptide that inhibits the interaction between CD47 and SIRPa. In one embodiment, this fusion protein comprises VEGFR ECD and a polypeptide that binds CD47. In one embodiment, this fusion protein is anti-CD47 mAb fused to VEGFR ECD (anti-CD47-VEGFR (e.g., SEQ ID NOs: 392, 22)). In another embodiment, this fusion protein comprises SIRPa ECD and VEGFR ECD. In one embodiment, this fusion protein is SIRPa-Fc-VEGFR (e.g., SEQ ID NO: 552) or VEGFR-Fc-SIRPa (e.g., SEQ ID NO: 568).

In a further aspect, the fusion protein comprises VEGFR ECD, a polypeptide that inhibits the interaction between CD47 and SIRPa, and another polypeptide that inhibits a T cell co-inhibitory molecule. In some embodiments, this fusion protein comprises anti-CD47 mAb with VEGFR ECD fused to the heavy chain; and a T cell co-inhibitory molecule ECD fused to the light chain. In a particular embodiment, this fusion protein is anti-CD47-VEGFR-PD1 (e.g., SEQ ID NOs: 392, 384).

In another aspect, the fusion protein comprises VEGFR ECD, SIRPa ECD, and an antibody with a heavy chain and light chain. In a preferred embodiment, the VEGFR ECD is fused to the heavy chain of the antibody and the SIRPa ECD is fused to the light chain of the antibody. In some embodiments, the antibody of said fusion protein binds a T cell co-inhibitory molecule as an antagonist. Exemplary embodiments of such fusion proteins include anti-CTLA4-VEGFR-SIRPa (e.g., SEQ ID NOs: 446, 438), anti-PD1-VEGFR-SIRPa (e.g., SEQ ID NOs: 458, 450), and anti-PDL1-VEGFR-SIRPa (e.g., SEQ ID NOs: 468, 461). In other embodiments, the antibody of said fusion protein binds a T cell co-stimulatory molecule as an agonist. Exemplary embodiments of this fusion protein include anti-OX40-VEGFR-SIRPa (e.g., SEQ ID NOs: 516, 508), anti-41BB-VEGFR-SIRPa (e.g., SEQ ID NOs: 504, 496), and anti-CD40-VEGFR-SIRPa. In other embodiments, the antibody of said fusion protein is a tumor-targeted antibody. In some embodiments, this tumor targeted-antibody binds a tumor growth factor or growth factor receptor. In other embodiments, this tumor targeted antibody binds a tumor cell surface molecule. Exemplary embodiments of this fusion protein include anti-EGFR-VEGFR-SIRPa (e.g., SEQ ID NOs: 231, 223), anti-HER2-VEGFR-SIRPa (e.g., SEQ ID NOs: 255, 247), anti-EGFRvIII-VEGFR-SIRPa (e.g., SEQ ID NOs: 243, 235), anti-uPAR-VEGFR-SIRPa, and anti-PSMA-VEGFR-SIRPa.

In other embodiments, the antibody of said fusion protein binds a member of the TGFb pathway. In some embodiments, this antibody binds TGFb, TGFbR, or GARP. Exemplary embodiments of this fusion protein include anti-TGFb-VEGFR-SIRPa (e.g., SEQ ID NOs: 403, 396), anti-TGFbR-VEGFR-SIRPa, and anti-GARP-VEGFR-SIRPa.

In some embodiments, the fusion protein comprises VEGFR ECD and a polypeptide that inhibits a T cell co-inhibitory molecule. In specific embodiments, the fusion protein comprises VEGFR ECD and an antibody that binds and disables a T cell co-inhibitory molecule.

In some embodiments, the fusion protein comprises VEGFR ECD and a polypeptide that inhibits the interaction of TIGIT or PVRIG with PVRL2 or PVR. In some embodiments, this fusion protein comprises an antibody that binds TIGIT or PVRIG fused to VEGFR ECD. Exemplary embodiments of this fusion protein include anti-TIGIT-VEGFR (e.g., SEQ ID NOs: 480, 139) and anti-PVRIG-VEGFR.

In some embodiments, the fusion protein comprises VEGFR ECD and a polypeptide that inhibits the interaction of VISTA and VSIG8. In some embodiments, this fusion protein comprises an antibody that binds VISTA or VSIG8 fused to VEGFR ECD. Exemplary embodiments of this fusion protein include anti-VISTA-VEGFR and anti-VSIG8-VEGFR.

In some embodiments, the fusion protein comprises VEGFR ECD and a polypeptide that inhibits the interaction of PD-1 and PD-L1. In some embodiments, this fusion protein comprises an antibody that binds PD-1 or PD-L1 fused to VEGFR ECD. Exemplary embodiments of this fusion protein include anti-PD1-VEGFR (e.g., SEQ ID NOs: 458, 101) and anti-PDLL-VEGFR (e.g., SEQ ID NOs: 468, 109).

In some embodiments, the fusion protein comprises VEGFR ECD and a polypeptide that inhibits CTLA-4. In some embodiments, this fusion protein comprises an antibody that binds CTLA-4 fused to VEGFR ECD. Exemplary embodiments of this fusion protein include anti-CTLA4-VEGFR (e.g., SEQ ID NOs: 446, 28).

In some embodiments, the fusion protein comprises VEGFR ECD, an antibody that binds and disables a T cell co-inhibitory molecule, and an additional receptor ECD. In one aspect, the VEGFR ECD is fused to the heavy chain of the antibody and the additional receptor ECD is fused to the light chain of the antibody. Alternatively, the VEGFR ECD is fused to the light chain and the additional receptor ECD is fused to the heavy chain.

In some embodiments, the fusion protein comprises VEGFR ECD, an antibody that binds and disables a T cell co-inhibitory molecule, and PD1 ECD. Exemplary embodiments of this fusion protein include anti-CTLA4-VEGFR-PD1 (e.g., SEQ ID NOs: 446, 436); anti-PD1-VEGFR-PD1 (e.g., SEQ ID NOs: 458, 448); anti-TIGIT-VEGFR-PD1 (e.g., SEQ ID NOs: 480, 470); anti-TIM3-VEGFR-PD1 (e.g., SEQ ID NOs: 492, 482).

In other embodiments, the fusion protein comprises VEGFR ECD, an antibody that binds and disables a T cell co-inhibitory molecule, and SIRPa ECD. Exemplary embodiments of this fusion protein include anti-PDLL-VEGFR-SIRPa (e.g., SEQ ID NOs: 468, 461); anti-PD1-VEGFR-SIRPa (e.g., SEQ ID NOs: 458, 450); anti-CTLA4-VEGFR-SIRPa (e.g., SEQ ID NOs: 446, 438); anti-TIGIT-VEGFR-SIRPa (e.g., SEQ ID NOs: 480, 472); anti-TIM3-VEGFR-SIRPa (e.g., SEQ ID NOs: 492, 484).

In other embodiments, the fusion protein comprises VEGFR ECD, an antibody that binds and disables a T cell co-inhibitory molecule, and SIGLEC10 ECD. Exemplary embodiments of this fusion protein include anti-TIGIT-VEGFR-SIGLEC10 (e.g., SEQ ID NOs: 480, 471); anti-CTLA4-VEGFR-SIGLEC10 (e.g., SEQ ID NOs: 446, 437); anti-PD1-VEGFR-SIGLEC10 (e.g., SEQ ID NOs: 458, 449); anti-TIM3-VEGFR-SIGLEC10 (e.g., SEQ ID NOs: 492, 483); anti-PDL1-VEGFR-SIGLEC10 (e.g., SEQ ID NOs: 468, 460).

In some embodiments, the fusion protein comprises an antibody that binds a T cell co-stimulatory molecule fused to VEGFR. This antibody is preferably an agonist of the T cell co-stimulatory molecule. In some embodiments, this fusion protein is selected from: anti-OX40-VEGFR (e.g., SEQ ID NOs: 516, 97); anti-41BB-VEGFR (e.g., SEQ ID NOs: 504, 2); anti-ICOS-VEGFR (e.g., SEQ ID NOs: 528, 59).

In a further aspect, the fusion protein comprises an antibody that binds a T cell-costimulatory molecule, VEGFR ECD, and an additional receptor ECD. In a preferred embodiment, the VEGFR ECD is fused to the heavy chain of the antibody and the additional receptor ECD is fused to the light chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-41BB-VEGFR-PD1 (e.g., SEQ ID NOs: 504, 494); anti-OX40-VEGFR-PD1 (e.g., SEQ ID NOs: 516, 506); anti-ICOS-VEGFR-PD1 (e.g., SEQ ID NOs: 528, 518). In another aspect, the additional receptor ECD is SIRPa ECD. In some embodiments, the fusion protein is selected from: anti-41BB-VEGFR-SIRPa (e.g., SEQ ID NOs: 504, 496); anti-ICOS-VEGFR-SIRPa (e.g., SEQ ID NOs: 528, 520); anti-OX40-VEGFR-SIRPa (e.g., SEQ ID NOs: 516, 508). In another aspect, the additional receptor ECD is SIGLEC10 ECD. In some embodiments, the fusion protein is selected from: anti-OX40-VEGFR-SIGLEC10 (e.g., SEQ ID NOs: 516, 507); anti-41BB-VEGFR-SIGLEC10 (e.g., SEQ ID NOs: 504, 495); anti-ICOS-VEGFR-SIGLEC10 (e.g., SEQ ID NOs: 528, 519).

In some embodiments, the fusion protein comprises an antibody, VEGFR ECD, and the ECD of a T cell co-stimulatory molecule. In a preferred embodiment, the VEGFR ECD is fused to heavy chain and the ECD of a T cell co-stimulatory molecule fused to light chain. In some embodiments, the fusion protein comprises VEGFR ECD and one of the following: OX40L, 41BBL, ICOSL. Exemplary embodiments of these fusion proteins include the following: VEGFR-Fc-41BBL (e.g., SEQ ID NO: 631); VEGFR-Fc-ICOSL (e.g., SEQ ID NO: 641); VEGFR-Fc-OX40L (e.g., SEQ ID NO: 645) and 41BBL-Fc-VEGFR (e.g., SEQ ID NO: 632); OX40L-Fc-VEGFR (e.g., SEQ ID NO: 646); ICOSL-Fc-VEGFR (e.g., SEQ ID NO: 642).

In one embodiment, the fusion protein comprises VEGFR ECD and a polypeptide that binds an ectonucleotidase. In a preferred embodiment, the ectonucleotidase is either CD39 or CD73. In some embodiments, the fusion protein is an antibody that binds CD39 or CD73 fused to VEGFR ECD; for example: anti-CD39-VEGFR (e.g., SEQ ID NOs: 434, 18) or anti-CD73-VEGFR (e.g., SEQ ID NOs: 427, 24).

In some embodiments, the fusion protein comprises VEGFR ECD, an antibody that binds CD39 or CD73, and an additional receptor ECD. In a preferred embodiment, the VEGFR ECD is fused to the heavy chain of the antibody and the additional receptor ECD is fused to the light chain of the antibody. In some embodiments, the fusion protein is selected from the following: anti-CD73-VEGFR-SIRPa (e.g., SEQ ID NOs: 427, 419); anti-CD73-VEGFR-PD1 (e.g., SEQ ID NOs: 427, 417).

In some embodiments, the fusion protein comprises a tumor-targeted antibody and VEGFR ECD. In some embodiments, this tumor targeted-antibody binds a tumor growth factor or growth factor receptor. In other embodiments, this tumor targeted antibody binds a tumor cell surface molecule. In some embodiments, this fusion protein is selected from the following: anti-EGFR-VEGFR (e.g., SEQ ID NOs: 231, 43), anti-HER2-VEGFR (e.g., SEQ ID NOs: 255, 55), anti-EGFRvIII-VEGFR (e.g., SEQ ID NOs: 243, 47), anti-uPAR-VEGFR (e.g., SEQ ID NOs: 274, 162), anti-PSMA-VEGFR (e.g., SEQ ID NOs: 281, 121), anti-nectin-4-VEGFR.

In a further embodiment, the fusion protein comprises a tumor-targeted antibody, VEGFR ECD, and an additional receptor ECD. In a preferred embodiment, the VEGFR ECD is fused to the heavy chain of the antibody and the additional receptor ECD is fused to the light chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-EGFRvIII-VEGFR-PD1 (e.g., SEQ ID NOs: 243, 233); anti-HER2-VEGFR-PD1 (e.g., SEQ ID NOs: 255, 245); anti-EGFR-VEGFR-PD1 (e.g., SEQ ID NOs: 231, 221); anti-nectin4-VEGFR-PD1 (e.g., SEQ ID NOs: 267, 257). In another aspect, the additional receptor ECD is SIRPa ECD. In some embodiments, the fusion protein is selected from: anti-EGFR-VEGFR-SIRPa (e.g., SEQ ID NOs: 231, 223); anti-nectin4-VEGFR-SIRPa (e.g., SEQ ID NOs: 267, 259); anti-HER2-VEGFR-SIRPa (e.g., SEQ ID NOs: 255, 247); anti-EGFRvIII-VEGFR-SIRPa (e.g., SEQ ID NOs: 243, 235). In another aspect, the additional receptor ECD is SIGLEC10 ECD. In some embodiments, the fusion protein is selected from: anti-EGFR-VEGFR-SIGLEC10 (e.g., SEQ ID NOs: 231, 222); anti-EGFRvIII-VEGFR-SIGLEC10 (e.g., SEQ ID NOs: 243, 234); anti-HER2-VEGFR-SIGLEC10 (e.g., SEQ ID NOs: 255, 246); anti-nectin4-VEGFR-SIGLEC10 (e.g., SEQ ID NOs: 267, 258).

In some embodiments, the fusion protein comprises an antibody that binds IL-17 or IL-17R and VEGFR ECD. In some embodiments, this fusion protein is selected from the following: anti-IL17-VEGFR, anti-IL17R-VEGFR (e.g., SEQ ID NOs: 336, 63).

In a further embodiment, the fusion protein comprises an antibody that binds IL-17 or IL-17R, VEGFR ECD, and an additional receptor ECD. In one embodiment, VEGFR ECD is fused to the heavy chain of the antibody and the additional receptor ECD is fused to the light chain of the antibody. Alternatively, VEGFR ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL17R-VEGFR-PD1 (e.g., SEQ ID NOs: 336, 326). In another aspect, the additional receptor ECD is SIRPa ECD. In some embodiments, the fusion protein is selected from: anti-IL17R-VEGFR-SIRPa (e.g., SEQ ID NOs: 336, 328). In another aspect, the additional receptor ECD is SIGLEC10 ECD. In some embodiments, the fusion protein is selected from: anti-IL17R-VEGFR-SIGLEC10 (e.g., SEQ ID NOs: 336, 327).

In some embodiments, the fusion protein comprises an antibody that binds IL-23 or IL-23R and VEGFR ECD. If the antibody binds IL-23, it is preferred that the antibody bind the p19 subunit of IL-23 that is not shared with IL-12. In some embodiments, this fusion protein is selected from the following: anti-IL23-VEGFR (e.g., SEQ ID NOs: 348, 75), anti-IL23R-VEGFR.

In a further embodiment, the fusion protein comprises an antibody that binds IL-23 or IL-23R, VEGFR ECD, and an additional receptor ECD. In one embodiment, VEGFR ECD is fused to the heavy chain of the antibody and the additional receptor ECD is fused to the light chain of the antibody. Alternatively, VEGFR ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL23-VEGFR-PD1 (e.g., SEQ ID NOs: 348, 338). In another aspect, the additional receptor ECD is SIRPa ECD. In some embodiments, the fusion protein is selected from: anti-IL23-VEGFR-SIRPa (e.g., SEQ ID NOs: 348, 340). In another aspect, the additional receptor ECD is SIGLEC10 ECD. In some embodiments, the fusion protein is selected from: anti-IL23-VEGFR-SIGLEC10 (e.g., SEQ ID NOs: 348, 339).

In some embodiments, the fusion protein comprises an antibody that binds IL-6 or IL-6R and VEGFR ECD. In some embodiments, this fusion protein is selected from the following: anti-IL6-VEGFR, anti-IL6R-VEGFR (e.g., SEQ ID NOs: 324, 79).

In some embodiments, the fusion protein comprises an antibody that binds IL-6 or IL-6R, VEGFR ECD, and an additional receptor ECD. In one embodiment, VEGFR ECD is fused to the heavy chain of the antibody and the additional receptor ECD is fused to the light chain of the antibody. Alternatively, VEGFR ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL6R-VEGFR-PD1 (e.g., SEQ ID NOs: 324, 314). In another aspect, the additional receptor ECD is SIRPa ECD. In some embodiments, the fusion protein is selected from: anti-IL6R-VEGFR-SIRPa (e.g., SEQ ID NOs: 324, 316). In another aspect, the additional receptor ECD is SIGLEC10 ECD. In some embodiments, the fusion protein is selected from: anti-IL6R-VEGFR-SIGLEC10 (e.g., SEQ ID NOs: 324, 315).

In another embodiment, the ALT comprises an antibody that targets TGFb or TGFbR or GARP or LAP, wherein said antibody is fused to a ligand-binding sequence of an extracellular domain of VEGFR (VEGFR ECD). In another aspect an ALT comprises an antibody that targets TGFb or TGFbR, wherein said antibody is fused to a ligand-binding sequence of a VEGFR ECD, and additionally fused to a ligand-binding sequence of another different receptor ECD (receptor ECD-2). In various examples, receptor ECD-2 may be selected from a group comprising PD-1ECD, TIM-3ECD, BTLA ECD, or SIRPa. In one aspect, the VEGFR ECD sequence is fused to the C-terminus of the heavy chain of the targeting antibody, and another different receptor ECD-2 sequence is fused to the C-terminus of the light chain. In one aspect, the receptor ECD-2 fused to a TGFb or TGFbR targeting antibody binds to a cognate ligand expressed in the TME, thereby localizing the ALT and consequent VEGF/VEGFR and TGFb blockade to the TME. In one example, the receptor ECD-2 is a PD-1ECD sequence that binds PD-L1 or PD-L2 expressed on tumor cells or the TME. In another example, the receptor ECD-2 is a TIM-3ECD sequence that binds CEACAM-1 or CEACAM-5 or CEACAM-6 expressed on tumor cells or the TME. In another example, receptor ECD-2 is a BTLA ECD that binds HVEM expressed on tumor cells or the TME. In another example, the receptor ECD-2 is a SIRPa ECD sequence that binds CD47 expressed on tumor cells or the TME.

In some embodiments, the fusion protein comprises VEGFR ECD and a polypeptide that binds TGFb, TGFbR, or GARP. Exemplary embodiments of this fusion protein include anti-TGFb-VEGFR (e.g., SEQ ID NOs: 403, 133), anti-TGFbR-VEGFR, and anti-GARP-VEGFR (e.g., SEQ ID NOs: 415, 49).

In some embodiments, the fusion protein further comprises an additional receptor ECD. Exemplary embodiments of this fusion protein include anti-TGFb-VEGFR-SIRPa (e.g., SEQ ID NOs: 403, 396); anti-TGFb-VEGFR-SIGLEC10 (e.g., SEQ ID NOs: 403, 395); anti-TGFb-VEGFR-BTLA (e.g., SEQ ID NOs: 403, 399); anti-TGFb-VEGFR-TIM3 (e.g., SEQ ID NOs: 403, 397); anti-TGFb-VEGFR-PD1 (e.g., SEQ ID NOs: 403, 394).

In some embodiments, the fusion protein comprises VEGFR ECD and TGFbRII ECD. Exemplary embodiments of this fusion protein include VEGFR-Fc-TGFbRII (e.g., SEQ ID NO: 569) and TGFbRII-Fc-VEGFR (e.g., SEQ ID NO: 558).

In some embodiments, the fusion protein comprises VEGFR ECD and IL-15. In some embodiments, the fusion protein is IL15-Fc-VEGFR (e.g., SEQ ID NO: 598) or VEGFR-Fc-IL15 (e.g., SEQ ID NO: 597). In other embodiments, the fusion protein is IL12-Fc-VEGFR (e.g., SEQ ID NO: 596) or VEGFR-Fc-IL12 (e.g., SEQ ID NO: 595). In other embodiments, the fusion protein comprises an antibody with VEGFR ECD fused to heavy chain and IL-15 fused to light chain. In other embodiments, the fusion protein comprises an antibody with VEGFR ECD fused to heavy chain and IL-12 fused to light chain.

In various embodiments, the fusion proteins of the invention counteract TGFb in the tumor microenvironment. These fusion proteins are referred to as belonging to “Group 2”. In some embodiments, the fusion proteins comprise a ligand-binding sequence of an extracellular domain of TGFbR (e.g., TGFbRII ECD). In one embodiment, this ligand trap binds TGFb1, TGFb2, and/or TGFb3. In some embodiments, the TGFbR ECD may be a ligand-binding sequence of TGFbRII ECD. In some embodiments, the TGFbR ECD may be a fusion of domains from TGFbRII and TGFbRIII. In some embodiments, the TGFbR ECD may be selected from the following list: SEQ ID NOS: 177; 178; 179; 180.

In other embodiments, the fusion proteins of the invention comprise a targeting polypeptide that binds TGFb, TGFbR, LAP, or GARP fused to one or more receptor ECDs. These receptor ECDs are preferably selected from the following: PD1 ECD, TIM-3 ECD, VEGFR ECD, BTLA ECD, SIRPa ECD, SIGLEC 10 ECD. In some embodiments, the targeting polypeptide that binds TGFb, TGFbR, LAP, or GARP is an antibody.

In some embodiments, the ALT is a polypeptide comprising an antibody that targets TGFb or TGFbR, wherein said antibody is fused to a ligand-binding sequence of an extracellular domain of a Receptor. In one example, the ALT comprises an antibody that targets TGFb, wherein said antibody is fused to a ligand-binding sequence of an extracellular domain of Receptor (e.g. PD-1ECD, TIM-3ECD, VEGFR ECD, BTLA ECD, SIRPa ECD). In another aspect an ALT comprises an antibody that targets TGFb, wherein said antibody is fused to a ligand-binding sequence of a specific receptor ECD (receptor ECD-1), and additionally fused to a ligand-binding sequence of another different receptor ECD (receptor ECD-2). In various examples, receptor ECD-1 and receptor ECD-2 may be selected from a group comprising PD-1ECD, TIM-3ECD, VEGFR ECD, BTLA ECD, SIRPa ECD, SIGLEC10 ECD. In one aspect, the receptor ECD-1 sequence is fused to the C-terminus of the heavy chain of the targeting antibody, and another different receptor ECD-2 sequence is fused to the C-terminus of the light chain. In one aspect, the receptor ECD fused to a TGFb targeting antibody binds to a cognate ligand expressed in the TME, thereby localizing the ALT and consequent TGFb blockade to the TME. In one example, a PD-1ECD binds PD-L1 or PD-L2 expressed on tumor cells or the TME. In another example, a TIM-3ECD binds CEACAM-1 or CEACAM-5 or CEACAM-6 expressed on tumor cells or the TME. In another example, a BTLA ECD binds HVEM expressed on tumor cells or the TME. In another example, a SIRPa ECD binds CD47 expressed on tumor cells or the TME. Exemplary embodiments include anti-TGFb-PD1 (e.g., SEQ ID NOs: 398, 133); anti-TGFb-SIRPa (e.g., SEQ ID NOs: 401, 133); anti-TGFb-TIM3 (e.g., SEQ ID NOs: 402, 133); anti-TGFb-SIGLEC10 (e.g., SEQ ID NOs: 400, 133); anti-TGFb-BTLA (e.g., SEQ ID NOs: 393, 133); anti-TGFb-VEGFR (e.g., SEQ ID NOs: 403, 133)

Although CD47 targeted antibodies can promote antitumor immune responses by inhibiting the interaction of CD47 with SIRPa, its antitumor efficacy may be limited by TSP-1 dependent or TSP-1 independent activation of TGFb. In one embodiment, the ALT is a polypeptide comprising an antibody that targets CD47, wherein said antibody is fused to a TGFb-binding sequence from a extracellular domain of TGFbR (e.g. TGFbRII ECD). In this aspect, the ALT promotes antitumor immunity by disrupting the interaction of CD47 with SIRPa, while simultaneously counteracting TGFb-mediated immune dysfunction and angiogenesis. In another aspect, the ALT comprises a TGFb-binding sequence from one or more extracellular domains of TGFbR (e.g. TGFbRII ECD) and a CD47-binding sequence from one or more extracellular domains of SIRPa (SIRPa ECD).

Examples of fusion proteins of the invention that capture and disable TGFb in the TME include, but are not limited to the following: ALTs comprising an antibody fused to TGFbRECD (with or without another receptor ECD fused to the same antibody); ALTs where the antibody binds TGFb, TGFbR, LAP, or GARP, fused to one or more Receptor ECD(s).

In one aspect, the ALT is a polypeptide comprising an antibody fused to a TGFb-binding sequence from an extracellular domain of TGFbR (e.g. TGFbRII ECD). The TGFbRECD fused to the ALT localizes to the TME, where it serves as a decoy receptor to bind and disable TGFb (e.g TGFb1, TGFb2, TGFb3). In another aspect an ALT comprising a fused ligand-binding sequence of TGFbRECD is additionally fused to a different receptor ECD that captures and disables its cognate ligands (e.g. PD-1ECD, TIM-3ECD, VEGFR ECD, BTLA ECD, SIRPa ECD, SIGLEC10 ECD). In one aspect, the TGFbRECD is fused to the C-terminus of the heavy chain of the targeting antibody, and another different receptor ECD is fused to the C-terminus of the light chain. In one example, the TGFbRECD is fused to the C-terminus of the heavy chain of the targeting antibody, and a PD-1 ligand-binding sequence of PD-1ECD is fused to the C-terminus of the light chain. In another example, the TGFbRECD is fused to the C-terminus of the heavy chain of the targeting antibody, and a TIM-3 ligand-binding sequence of TIM-3ECD is fused to the C-terminus of the light chain. In another example, the TGFbRECD is fused to the C-terminus of the heavy chain of the targeting antibody, and a BTLA ligand-binding sequence of BTLA ECD is fused to the C-terminus of the light chain. In another example, the TGFbRECD is fused to the C-terminus of the heavy chain of the targeting antibody, and a CD47 ligand-binding sequence of SIRPa ECD is fused to the C-terminus of the light chain.

In some embodiments, the fusion protein comprises TGFbRII ECD and a polypeptide that inhibits the interaction between CD47 and SIRPa. In one embodiment, this fusion protein comprises TGFbRII ECD and a polypeptide that binds CD47. In one embodiment, this fusion protein is anti-CD47 mAb fused to TGFbRII ECD (anti-CD47-TGFbRII (e.g., SEQ ID NOs: 390, 22)). In another embodiment, this fusion protein comprises SIRPa ECD and TGFbRII ECD. In one embodiment, this fusion protein is SIRPa-Fc-TGFbRII (e.g., SEQ ID NO: 550) or TGFbRII-Fc-SIRPa (e.g., SEQ ID NO: 556).

In some embodiments, the fusion protein comprises TGFbRII ECD, a polypeptide that inhibits the interaction between CD47 and SIRPa, and another polypeptide. In a further embodiment, the fusion protein comprises TGFbRII ECD, a polypeptide that inhibits the interaction between CD47 and SIRPa, and another polypeptide that inhibits a T cell co-inhibitory molecule. In some embodiments, this fusion protein comprises anti-CD47 mAb with TGFbRII ECD fused to the heavy chain and a T cell co-inhibitory molecule ECD fused to the light chain. In a particular embodiment, this fusion protein is anti-CD47-TGFbRII-PD1 (e.g., SEQ ID NOs: 390, 384). In another embodiment, this fusion protein is anti-CD47-TGFbRII-BTLA (e.g., SEQ ID NOs: 390, 388). In another embodiment, this fusion protein is anti-CD47-TGFbRII-TIM3 (e.g., SEQ ID NOs: 390, 386).

In other embodiments, the fusion protein comprises TGFbRII ECD, SIRPa ECD, and an antibody with a heavy chain and light chain. In one embodiment, the TGFbRII ECD is fused to the heavy chain of the antibody and the SIRPa ECD is fused to the light chain of the antibody. In some embodiments, the antibody of said fusion protein binds a T cell co-inhibitory molecule as an antagonist. Exemplary embodiments of such fusion proteins include anti-CTLA4-TGFbRII-SIRPa (e.g., SEQ ID NOs: 444, 438), anti-PD1-TGFbRII-SIRPa (e.g., SEQ ID NOs: 456, 450), and anti-PDL1-TGFbRII-SIRPa (e.g., SEQ ID NOs: 466, 461). In other embodiments, the antibody of said fusion protein binds a T cell co-stimulatory molecule as an agonist. Exemplary embodiments of this fusion protein include anti-OX40-TGFbRII-SIRPa (e.g., SEQ ID NOs: 514, 508), anti-41BB-TGFbRII-SIRPa (e.g., SEQ ID NOs: 502, 496), and anti-CD40-TGFbRII-SIRPa. In other embodiments, the antibody of said fusion protein is a tumor-targeted antibody. In some embodiments, this tumor targeted-antibody binds a tumor growth factor or growth factor receptor. In other embodiments, this tumor targeted antibody binds a tumor cell surface molecule. Exemplary embodiments of this fusion protein include anti-EGFR-TGFbRII-SIRPa (e.g., SEQ ID NOs: 229, 223), anti-HER2-TGFbRII-SIRPa (e.g., SEQ ID NOs: 253, 247), anti-EGFRvIII-TGFbRII-SIRPa (e.g., SEQ ID NOs: 241, 235), anti-uPAR-TGFbRII-SIRPa, and anti-PSMA-TGFbRII-SIRPa.

In some embodiments, the fusion protein comprises TGFbRII ECD and a polypeptide that inhibits the interaction of BTLA and HVEM. In some embodiments, this fusion protein comprises an antibody that binds BTLA or HVEM fused to TGFbRII ECD. Exemplary embodiments of this fusion protein include anti-BTLA-TGFbRII and anti-HVEM-TGFbRII, BTLA-Fc-TGFbRII (e.g., SEQ ID NO: 532), and TGFbRII-Fc-BTLA (e.g., SEQ ID NO: 553).

In some embodiments, the fusion protein comprises TGFbRII ECD and a polypeptide that inhibits the interaction of TIGIT or PVRIG with PVRL2 or PVR. In some embodiments, this fusion protein comprises an antibody that binds TIGIT or PVRIG fused to TGFbRII ECD. Exemplary embodiments of this fusion protein include anti-TIGIT-TGFbRII (e.g., SEQ ID NOs: 478, 139) and anti-PVRIG-TGFbRII.

In some embodiments, the fusion protein comprises TGFbRII ECD and a polypeptide that inhibits the interaction of VISTA and VSIG8. In some embodiments, this fusion protein comprises an antibody that binds VISTA or VSIG8 fused to TGFbRII ECD. Exemplary embodiments of this fusion protein include anti-VISTA-TGFbRII and anti-VSIG8-TGFbRII.

In some embodiments, the fusion protein comprises TGFbRII ECD, an antibody that binds and disables a T cell co-inhibitory molecule, and an additional receptor ECD. In one aspect, the TGFbRII ECD is fused to the heavy chain of the antibody and the additional receptor ECD is fused to the light chain of the antibody.

In some embodiments, the fusion protein comprises TGFbRII ECD, an antibody that binds and disables a T cell co-inhibitory molecule, and PD1 ECD. Exemplary embodiments of this fusion protein include anti-TIGIT-TGFbRII-PD1 (e.g., SEQ ID NOs: 478, 470); anti-TIM3-TGFbRII-PD1 (e.g., SEQ ID NOs: 490, 482); anti-PD1-TGFbRII-PD1 (e.g., SEQ ID NOs: 456, 448); anti-CTLA4-TGFbRII-PD1 (e.g., SEQ ID NOs: 444, 436).

In other embodiments, the fusion protein comprises TGFbRII ECD, an antibody that binds and disables a T cell co-inhibitory molecule, and BTLA ECD. Exemplary embodiments of this fusion protein include anti-TIGIT-TGFbRII-BTLA (e.g., SEQ ID NOs: 478, 475); anti-PDL1-TGFbRII-BTLA (e.g., SEQ ID NOs: 466, 464); anti-CTLA4-TGFbRII-BTLA (e.g., SEQ ID NOs: 444, 441); anti-PD1-TGFbRII-BTLA (e.g., SEQ ID NOs: 456, 453); anti-TIM3-TGFbRII-BTLA (e.g., SEQ ID NOs: 490, 487).

In other embodiments, the fusion protein comprises TGFbRII ECD, an antibody that binds and disables a T cell co-inhibitory molecule, and TIM3 ECD. Exemplary embodiments of this fusion protein include anti-TIGIT-TGFbRII-TIM3 (e.g., SEQ ID NOs: 478, 473); anti-TIM3-TGFbRII-TIM3 (e.g., SEQ ID NOs: 490, 485); anti-CTLA4-TGFbRII-TIM3 (e.g., SEQ ID NOs: 444, 439); anti-PD1-TGFbRII-TIM3 (e.g., SEQ ID NOs: 456, 451); anti-PDL1-TGFbRII-TIM3 (e.g., SEQ ID NOs: 466, 462).

In other embodiments, the fusion protein comprises TGFbRII ECD, an antibody that binds and disables a T cell co-inhibitory molecule, and SIRPa ECD. Exemplary embodiments of this fusion protein include anti-PDL1-TGFbRII-SIRPa (e.g., SEQ ID NOs: 466, 461); anti-CTLA4-TGFbRII-SIRPa (e.g., SEQ ID NOs: 444, 438); anti-TIM3-TGFbRII-SIRPa (e.g., SEQ ID NOs: 490, 484); anti-PD1-TGFbRII-SIRPa (e.g., SEQ ID NOs: 456, 450); anti-TIGIT-TGFbRII-SIRPa (e.g., SEQ ID NOs: 478, 472).

In other embodiments, the fusion protein comprises TGFbRII ECD, an antibody that binds and disables a T cell co-inhibitory molecule, and SIGLEC10 ECD. Exemplary embodiments of this fusion protein include anti-CTLA4-TGFbRII-SIGLEC10 (e.g., SEQ ID NOs: 444, 437); anti-PDL1-TGFbRII-SIGLEC10 (e.g., SEQ ID NOs: 466, 460); anti-TIM3-TGFbRII-SIGLEC10 (e.g., SEQ ID NOs: 490, 483); anti-PD1-TGFbRII-SIGLEC10 (e.g., SEQ ID NOs: 456, 449); anti-TIGIT-TGFbRII-SIGLEC10 (e.g., SEQ ID NOs: 478, 471).

In one embodiment, the fusion protein comprises a polypeptide that binds a T cell co-stimulatory molecule and TGFbRII ECD.

In some embodiments, the fusion protein is a native T cell co-stimulatory molecule ECD fused to TGFbRII (either N-costimulatory ECD-Fc-TGFbRII ECD-C, or N-TGFbRII ECD-Fc-costimulatory ECD-C). In some embodiments, this fusion protein is selected from: 41BBL-Fc-TGFbRII (e.g., SEQ ID NO: 616); ICOSL-Fc-TGFbRII (e.g., SEQ ID NO: 626); OX40L-Fc-TGFbRII (e.g., SEQ ID NO: 630), TGFbRII-Fc-ICOSL (e.g., SEQ ID NO: 625); TGFbRII-Fc-OX40L (e.g., SEQ ID NO: 629); TGFbRII-Fc-41BBL (e.g., SEQ ID NO: 615).

In other embodiments, the fusion protein comprises an antibody or other polypeptide that binds a T cell co-stimulatory molecule fused to TGFbRII. This antibody or polypeptide is preferably an agonist of the T cell co-stimulatory molecule. In some embodiments, this fusion protein is selected from: anti-ICOS-TGFbRII (e.g., SEQ ID NOs: 526, 59); anti-OX40-TGFbRII (e.g., SEQ ID NOs: 514, 97); anti-41BB-TGFbRII (e.g., SEQ ID NOs: 502, 2).

In a further aspect, the fusion protein comprises an antibody that binds a T cell-costimulatory molecule, TGFbRII ECD, and an additional receptor ECD. In one aspect, the TGFbRII ECD is fused to the heavy chain of the antibody and the additional receptor ECD is fused to the light chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-41BB-TGFbRII-PD1 (e.g., SEQ ID NOs: 502, 494); anti-OX40-TGFbRII-PD1 (e.g., SEQ ID NOs: 514, 506); anti-ICOS-TGFbRII-PD1 (e.g., SEQ ID NOs: 526, 518). In another embodiment, the T cell co-inhibitory molecule is BTLA. In some embodiments, the fusion protein is selected from: anti-OX40-TGFbRII-BTLA (e.g., SEQ ID NOs: 514, 511); anti-41BB-TGFbRII-BTLA (e.g., SEQ ID NOs: 502, 499); anti-ICOS-TGFbRII-BTLA (e.g., SEQ ID NOs: 526, 523). In another embodiment, the T cell co-inhibitory molecule is TIM3. In some embodiments, the fusion protein is selected from: anti-41BB-TGFbRII-TIM3 (e.g., SEQ ID NOs: 502, 497); anti-OX40-TGFbRII-TIM3 (e.g., SEQ ID NOs: 514, 509); anti-ICOS-TGFbRII-TIM3 (e.g., SEQ ID NOs: 526, 521). In another aspect, the additional receptor ECD is SIRPa ECD. In some embodiments, the fusion protein is selected from: anti-ICOS-TGFbRII-SIRPa (e.g., SEQ ID NOs: 526, 520); anti-41BB-TGFbRII-SIRPa (e.g., SEQ ID NOs: 502, 496); anti-OX40-TGFbRII-SIRPa (e.g., SEQ ID NOs: 514, 508). In another aspect, the additional receptor ECD is SIGLEC10 ECD. In some embodiments, the fusion protein is selected from: anti-41BB-TGFbRII-SIGLEC10 (e.g., SEQ ID NOs: 502, 495); anti-ICOS-TGFbRII-SIGLEC10 (e.g., SEQ ID NOs: 526, 519); anti-OX40-TGFbRII-SIGLEC10 (e.g., SEQ ID NOs: 514, 507).

In one embodiment, the fusion protein comprises TGFbRII ECD and a polypeptide that binds an ectonucleotidase. In a preferred embodiment, the ectonucleotidase is either CD39 or CD73. In some embodiments, the fusion protein comprises TGFbRII ECD, an antibody that binds CD39 or CD73, and an additional receptor ECD. In a preferred embodiment, the TGFbRII ECD is fused to the heavy chain of the antibody and the additional receptor ECD is fused to the light chain of the antibody. In some embodiments, the fusion protein is selected from the following: anti-CD73-TGFbRII-SIRPa (e.g., SEQ ID NOs: 425, 419); anti-CD73-TGFbRII-SIGLEC10 (e.g., SEQ ID NOs: 425, 418); anti-CD73-TGFbRII-BTLA (e.g., SEQ ID NOs: 425, 422); anti-CD73-TGFbRII-PD1 (e.g., SEQ ID NOs: 425, 417); anti-CD73-TGFbRII-TIM3 (e.g., SEQ ID NOs: 425, 420).

In some embodiments, the fusion protein comprises a tumor-targeted antibody and TGFbRII ECD. In some embodiments, this tumor targeted-antibody binds a tumor growth factor or growth factor receptor. In other embodiments, this tumor targeted antibody binds a tumor cell surface molecule. In some embodiments, this fusion protein is selected from the following: anti-EGFRvIII-TGFbRII (e.g., SEQ ID NOs: 241, 47), anti-uPAR-TGFbRII (e.g., SEQ ID NOs: 272, 162), anti-PSMA-TGFbRII (e.g., SEQ ID NOs: 279, 121), anti-nectin-4-TGFbRII.

In a further embodiment, the fusion protein comprises a tumor-targeted antibody, TGFbRII ECD, and an additional receptor ECD. In a preferred embodiment, the TGFbRII is fused to the heavy chain of the antibody and the additional receptor ECD is fused to the light chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-HER2-TGFbRII-PD1 (e.g., SEQ ID NOs: 253, 245); anti-EGFR-TGFbRII-PD1 (e.g., SEQ ID NOs: 229, 221); anti-nectin4-TGFbRII-PD1 (e.g., SEQ ID NOs: 265, 257); anti-EGFRvIII-TGFbRII-PD1 (e.g., SEQ ID NOs: 241, 233). In another embodiment, the T cell co-inhibitory molecule is BTLA. In some embodiments, the fusion protein is selected from: anti-HER2-TGFbRII-BTLA (e.g., SEQ ID NOs: 253, 250); anti-EGFR-TGFbRII-BTLA (e.g., SEQ ID NOs: 229, 226); anti-EGFRvIII-TGFbRII-BTLA (e.g., SEQ ID NOs: 241, 238); anti-nectin4-TGFbRII-BTLA (e.g., SEQ ID NOs: 265, 262). In another embodiment, the T cell co-inhibitory molecule is TIM3. In some embodiments, the fusion protein is selected from: anti-EGFR-TGFbRII-TIM3 (e.g., SEQ ID NOs: 229, 224); anti-EGFRvIII-TGFbRII-TIM3 (e.g., SEQ ID NOs: 241, 236); anti-HER2-TGFbRII-TIM3 (e.g., SEQ ID NOs: 253, 248); anti-nectin4-TGFbRII-TIM3 (e.g., SEQ ID NOs: 265, 260). In another aspect, the additional receptor ECD is SIRPa ECD. In some embodiments, the fusion protein is selected from: anti-EGFR-TGFbRII-SIRPa (e.g., SEQ ID NOs: 229, 223); anti-nectin4-TGFbRII-SIRPa (e.g., SEQ ID NOs: 265, 259); anti-HER2-TGFbRII-SIRPa (e.g., SEQ ID NOs: 253, 247); anti-EGFRvIII-TGFbRII-SIRPa (e.g., SEQ ID NOs: 241, 235). In another aspect, the additional receptor ECD is SIGLEC10 ECD. In some embodiments, the fusion protein is selected from: anti-nectin4-TGFbRII-SIGLEC10 (e.g., SEQ ID NOs: 265, 258); anti-HER2-TGFbRII-SIGLEC10 (e.g., SEQ ID NOs: 253, 246); anti-EGFRvIII-TGFbRII-SIGLEC10 (e.g., SEQ ID NOs: 241, 234); anti-EGFR-TGFbRII-SIGLEC10 (e.g., SEQ ID NOs: 229, 222).

In one aspect an ALT of the invention simultaneously counteracts VEGF and TGFb in the tumor microenvironment. In one example, the ALT comprises an antibody that targets either VEGF or VEGFR, wherein said antibody is fused to a TGFb-binding sequence of an extracellular domain of the TGFbR (e.g. TGFbRII ECD). In another aspect an ALT comprises an antibody that targets either VEGF or VEGFR, wherein said antibody is fused to a ligand-binding sequence of TGFbRECD, and additionally fused to a ligand-binding sequence of another different receptor ECD (receptor ECD-2). In various examples, receptor ECD-2 may be selected from a group comprising PD-1ECD, TIM-3ECD, BTLA ECD, or SIRPa ECD. In one aspect, the TGFb-binding TGFbRII ECD sequence is fused to the C-terminus of the heavy chain of the targeting antibody, and the receptor ECD-2 sequence is fused to the C-terminus of the light chain. In one aspect, the receptor ECD-2 sequence is fused to a VEGF or VEGFR targeting antibody; and binds to a cognate ligand expressed in the TME, thereby localizing the ALT and consequent VEGF/VEGFR and TGFb blockade to the TME. In one example, the receptor ECD-2 is a PD-1ECD sequence that binds PD-L1 or PD-L2 expressed on tumor cells or the TME. In another example, the receptor ECD-2 is a TIM-3ECD sequence that binds CEACAM-1 or CEACAM-5 or CEACAM-6 expressed on tumor cells or the TME. In another example, receptor ECD-2 is a BTLA ECD that binds HVEM expressed on tumor cells or the TME. In another example, the receptor ECD-2 is a SIRPa ECD sequence that binds CD47 expressed on tumor cells or the TME.

In some embodiments, the fusion protein comprises TGFbRII ECD and a polypeptide that inhibits VEGF/VEGFR signaling.

In some embodiments, the fusion protein comprises TGFbRII ECD and anti-VEGFR mAb. In some embodiments, this fusion protein is anti-VEGFR-TGFbRII (e.g., SEQ ID NOs: 381, 148). In a further embodiment, the fusion protein may additionally comprise a receptor ECD fused to the light chain of the antibody. In some embodiments, the fusion protein is selected from anti-VEGFR-TGFbRII-TIM3 (e.g., SEQ ID NOs: 381, 376); anti-VEGFR-TGFbRII-BTLA (e.g., SEQ ID NOs: 381, 378); anti-VEGFR-TGFbRII-SIGLEC10 (e.g., SEQ ID NOs: 381, 374); anti-VEGFR-TGFbRII-PD1 (e.g., SEQ ID NOs: 381, 373); anti-VEGFR-TGFbRII-SIRPa (e.g., SEQ ID NOs: 381, 375).

In some embodiments, the fusion protein comprises TGFbRII ECD and anti-VEGF mAb. In some embodiments, this fusion protein is anti-VEGF-TGFbRII (e.g., SEQ ID NOs: 370, 32). In a further embodiment, the fusion protein may additionally comprise a receptor ECD fused to the light chain of the antibody. In some embodiments, the fusion protein is selected from anti-VEGF-TGFbRII-TIM3 (e.g., SEQ ID NOs: 370, 365); anti-VEGF-TGFbRII-SIRPa (e.g., SEQ ID NOs: 370, 364); anti-VEGF-TGFbRII-PD1 (e.g., SEQ ID NOs: 370, 362); anti-VEGF-TGFbRII-BTLA (e.g., SEQ ID NOs: 370, 367); anti-VEGF-TGFbRII-SIGLEC10 (e.g., SEQ ID NOs: 370, 363).

In other embodiments, the fusion protein comprises TGFbRII ECD and VEGFR ECD. In one embodiment, this fusion protein is TGFbRII-Fc-VEGFR (e.g., SEQ ID NO: 558). In another embodiment, this fusion protein is VEGFR-Fc-TGFbRII (e.g., SEQ ID NO: 569).

In some embodiments, the fusion protein comprises an antibody that binds IL-17 or IL-17R and TGFbRII ECD. In some embodiments, this fusion protein is selected from the following: anti-IL17-TGFbRII, anti-IL17R-TGFbRII (e.g., SEQ ID NOs: 334, 63).

In a further embodiment, the fusion protein comprises an antibody that binds IL-17 or IL-17R, TGFbRII ECD, and an additional receptor ECD. In a preferred embodiment, the TGFbRII is fused to the heavy chain of the antibody and the additional receptor ECD is fused to the light chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-IL17R-TGFbRII-PD1 (e.g., SEQ ID NOs: 334, 326). In another embodiment, the T cell co-inhibitory molecule is BTLA. In some embodiments, the fusion protein is selected from: anti-IL17R-TGFbRII-BTLA (e.g., SEQ ID NOs: 334, 331). In another embodiment, the T cell co-inhibitory molecule is TIM3. In some embodiments, the fusion protein is selected from: anti-IL17R-TGFbRII-TIM3 (e.g., SEQ ID NOs: 334, 329). In another aspect, the additional receptor ECD is SIRPa ECD. In some embodiments, the fusion protein is selected from: anti-IL17R-TGFbRII-SIRPa (e.g., SEQ ID NOs: 334, 328). In another aspect, the additional receptor ECD is SIGLEC10 ECD. In some embodiments, the fusion protein is selected from: anti-IL17R-TGFbRII-SIGLEC10 (e.g., SEQ ID NOs: 334, 327).

In some embodiments, the fusion protein comprises an antibody that binds IL-23 or IL-23R and TGFbRII ECD. If the antibody binds IL-23, it is preferred that the antibody bind the p19 subunit of IL-23 that is not shared with IL-12. In some embodiments, this fusion protein is selected from the following: anti-IL23-TGFbRII (e.g., SEQ ID NOs: 346, 75), anti-IL23R-TGFbRII.

In a further embodiment, the fusion protein comprises an antibody that binds IL-23 or IL-23R, TGFbRII ECD, and an additional receptor ECD. In a preferred embodiment, the TGFbRII is fused to the heavy chain of the antibody and the additional receptor ECD is fused to the light chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-IL23-TGFbRII-PD1 (e.g., SEQ ID NOs: 346, 338). In another embodiment, the T cell co-inhibitory molecule is BTLA. In some embodiments, the fusion protein is selected from: anti-IL23-TGFbRII-BTLA (e.g., SEQ ID NOs: 346, 343). In another embodiment, the T cell co-inhibitory molecule is TIM3. In some embodiments, the fusion protein is selected from: anti-IL23-TGFbRII-TIM3 (e.g., SEQ ID NOs: 346, 341). In another aspect, the additional receptor ECD is SIRPa ECD. In some embodiments, the fusion protein is selected from: anti-IL23-TGFbRII-SIRPa (e.g., SEQ ID NOs: 346, 340). In another aspect, the additional receptor ECD is SIGLEC10 ECD. In some embodiments, the fusion protein is selected from: anti-IL23-TGFbRII-SIGLEC10 (e.g., SEQ ID NOs: 346, 339).

In some embodiments, the fusion protein comprises an antibody that binds IL-6 or IL-6R, TGFbRII ECD, and an additional receptor ECD. In a preferred embodiment, the TGFbRII is fused to the heavy chain of the antibody and the additional receptor ECD is fused to the light chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-IL6R-TGFbRII-PD1 (e.g., SEQ ID NOs: 322, 314). In another embodiment, the T cell co-inhibitory molecule is BTLA. In some embodiments, the fusion protein is selected from: anti-IL6R-TGFbRII-BTLA (e.g., SEQ ID NOs: 322, 319). In another embodiment, the T cell co-inhibitory molecule is TIM3. In some embodiments, the fusion protein is selected from: anti-IL6R-TGFbRII-TIM3 (e.g., SEQ ID NOs: 322, 317). In another aspect, the additional receptor ECD is SIRPa ECD. In some embodiments, the fusion protein is selected from: anti-IL6R-TGFbRII-SIRPa (e.g., SEQ ID NOs: 322, 316). In another aspect, the additional receptor ECD is SIGLEC10 ECD. In some embodiments, the fusion protein is selected from: anti-IL6R-TGFbRII-SIGLEC10 (e.g., SEQ ID NOs: 322, 315).

In some embodiments, the fusion protein comprises TGFbRII ECD and IL-15. In some embodiments, the fusion protein is IL15-Fc-TGFbRII (e.g., SEQ ID NO: 590) or TGFbRII-Fc-IL15 (e.g., SEQ ID NO: 589). In other embodiments, the fusion protein is IL12-Fc-TGFbRII (e.g., SEQ ID NO: 588) or TGFbRII-Fc-IL12 (e.g., SEQ ID NO: 587). In other embodiments, the fusion protein comprises an antibody with TGFbRII ECD fused to heavy chain and IL-15 fused to light chain. In other embodiments, the fusion protein comprises an antibody with TGFbRII ECD fused to heavy chain and IL-12 fused to light chain.

The data in Example 3 demonstrates that a fusion protein that blocks VEGF and also comprises the ECD of a molecule that inhibits immune cells (e.g., T cells, dendritic cells, macrophages) is effective in treating cancer. As such, in some embodiments of the invention, the fusion protein comprises a VEGF/VEGFR-blocking polypeptide and the ECD of an immuno-inhibitory receptor (e.g., SIRPa, SIGLEC10, PD1, BTLA, TIM-3). In other embodiments, the fusion protein comprises VEGFR ECD and an antibody that binds and disables the interaction of an immuno-inhibitory receptor and its ligand (e.g., anti-BTLA-VEGFR, anti-CD47-VEGFR, anti-PD1-VEGFR, anti-PDL1-VEGFR). In other embodiments, the fusion protein comprises VEGFR and the ECD of an immuno-inhibitory receptor (e.g., VEGFR-Fc-SIRPa, VEGFR-Fc-BTLA, VEGFR-Fc-SIGLEC10). In other embodiments, the fusion protein comprises anti-VEGF/VEGFR mAb and the ECD of an immuno-inhibitory receptor (e.g., anti-VEGF-SIRPa, anti-VEGF-BTLA, anti-VEGF-TIM3).

Furthermore, Example 3 demonstrates that a fusion protein that blocks VEGF and also comprises the ECD of a T cell co-inhibitory molecule is effective in treating cancer. As such, in some embodiments of the invention, the fusion protein comprises a VEGF/VEGFR-blocking polypeptide and the ECD of a T cell co-inhibitory molecule (e.g., PD1, BTLA, TIM-3).

Furthermore, Example 3 demonstrates that a fusion protein that blocks VEGF and also comprises a polypeptide that binds a tumor cell surface molecule or molecule enriched in the tumor microenvironment is effective in localizing VEGF to the tumor microenvironment. As such, in some embodiments of the invention, the fusion protein comprises a VEGF/VEGFR-blocking polypeptide and a polypeptide that binds a tumor cell surface molecule or molecule enriched in the tumor microenvironment. In some embodiments, this fusion protein comprises VEGFR ECD fused to an antibody that localizes to the TME (anti-nectin-4-VEGFR, anti-PSMA-VEGFR, anti-IL17R-VEGFR, anti-CD47-VEGFR). In other embodiments, this fusion protein comprises anti-VEGF/VEGFR antibody fused to a receptor ECD that localizes to the TME (e.g., anti-VEGF-BTLA, anti-VEGF-TIM3).

The data in Example 4 demonstrates that blockade of TGFb and VEGF is more effective in treatment of cancer than blockade of VEGF alone. As such, in some embodiments of the invention, the fusion protein comprises a polypeptide that inhibits TGFb and a polypeptide that inhibits VEGF. In some embodiments, this fusion protein comprises TGFbRII and VEGFR (e.g., TGFbRII-Fc-VEGFR). In other embodiments, the fusion protein comprises antibody that binds TGFb, TGFbR, LAP, or GARP and VEGFR (e.g., anti-TGFb-VEGFR). In other embodiments, the fusion protein comprises antibody that binds VEGF or VEGFR and TGFbRII ECD (e.g., anti-VEGF-TGFbRII, anti-VEGFR-TGFbRII).

Furthermore, Example 4 demonstrates that a fusion protein comprising an anti-VEGF/VEGFR polypeptide and another polypeptide that inhibits angiogenesis (e.g., TGFb) is effective in the treatment of cancer. As such, in some embodiments of the invention, the fusion protein comprises VEGFR ECD and a polypeptide that inhibits another determinant of angiogenesis. This additional determinant of angiogenesis may be TGFb, IL-17, or IL-17R. In some embodiments, this fusion protein is anti-TGFb-VEGFR, anti-IL17-VEGFR, anti-IL17R-VEGFR, or TGFbRII-Fc-VEGFR.

Furthermore, Example 4 demonstrates that a fusion protein comprising an antibody that inhibits angiogenesis fused to a receptor ECD that inhibits angiogenesis is effective in the treatment of cancer. As such, in some embodiments of the invention, the fusion protein comprises an antibody that inhibits angiogenesis (e.g., anti-VEGF, anti-VEGFR, anti-TGFb, anti-TGFbR, anti-IL-17, anti-IL17R) fused to a receptor ECD that inhibits angiogenesis (VEGFR ECD, TGFbRII ECD). Exemplary embodiments of these fusion proteins include anti-VEGF-TGFbRII, anti-IL17-TGFbRII, anti-IL17R-TGFbRII, anti-IL17-VEGFR, anti-IL17R-VEGFR, anti-TGFb-VEGFR.

Furthermore, Example 4 demonstrates that a fusion protein comprising anti-VEGF/VEGFR antibody and a receptor ECD that inhibits angiogenesis is effective in the treatment of cancer. As such, in some embodiments of the invention, the fusion protein comprises anti-VEGF/VEGFR mAb and a receptor ECD that inhibits angiogenesis. In some embodiments, this receptor ECD that inhibits angiogenesis is TGFbRII.

Furthermore, Example 4 demonstrates that a fusion protein comprising an anti-angiogenic polypeptide and a polypeptide that inhibits a key determinant of TH17 differentiation is effective in the treatment of cancer. As such, in some embodiments of the invention, the fusion protein comprises VEGFR and polypeptide that binds a key determinant of TH17 differentiation. In some embodiments, this key determinant of TH17 differentiation is TGFb/TGFbR, IL-6/IL-6R, IL-1/IL-1R, or IL-23/IL-23R. Exemplary embodiments of these fusion proteins include anti-IL23-TGFbRII, anti-IL23R-TGFbRII, anti-IL23-VEGFR, and anti-IL23R-VEGFR.

Furthermore, Example 4 demonstrates that localized blockade of VEGF and/or TGFb in the tumor microenvironment is effective in the treatment of cancer. As such, in some embodiments of the invention, a combination therapy of a tumor-localized inhibitor of VEGF is combined with an inhibitor of TGFb. In other embodiments, a combination therapy of a tumor-localized inhibitor of VEGF is combined with a tumor-localized inhibitor of TGFb. In other embodiments, a combination therapy of an inhibitor of VEGF is combined with a tumor-localized inhibitor of TGFb.

In some embodiments, tumor localization of VEGF inhibition is achieved via an ALT or ECD-ECD of the invention comprising VEGFR ECD and a polypeptide that binds a tumor cell surface molecule, cell surface molecule of a tumor-infiltrating immune cell, or other factor enriched in the tumor microenvironment. Exemplary embodiments of this agent include anti-CD47-VEGFR, anti-PDL1-VEGFR, anti-HER2-VEGFR, anti-EGFRvIII-VEGFR, anti-PSMA-VEGFR, anti-nectin-4-VEGFR. Further exemplary embodiments of this agent that localize to tumor-infiltrating T cells (e.g., tumor-infiltrating Tregs) include anti-CD39-VEGFR, anti-CD73-VEGFR, anti-CTLA4-VEGFR. In a further embodiment, the localizing polypeptide of the VEGFR-containing fusion protein is a receptor ECD. Exemplary embodiments of this fusion protein include VEGFR-Fc-SIRPa, VEGFR-Fc-BTLA, VEGFR-Fc-PD1, VEGFR-Fc-TIM3. In other embodiments, tumor localization of VEGF inhibition is achieved by an ALT of the invention comprising anti-VEGF/VEGFR mAb fused to a receptor ECD that binds a tumor cell or tumor-infiltrating immune cell. Exemplary embodiments of this agent include anti-VEGF-SIRPa, anti-VEGF-SIGLEC10, anti-VEGF-BTLA, anti-VEGF-TIM3, anti-VEGF-PD1.

In some embodiments, tumor localization of TGFb inhibition is achieved via an ALT or ECD-ECD of the invention comprising TGFbRII ECD and a polypeptide that binds a tumor cell surface molecule, cell surface molecule of a tumor-infiltrating immune cell, or other factor enriched in the tumor microenvironment. Exemplary embodiments of this agent include anti-CD47-TGFbRII, anti-PDL1-TGFbRII, anti-HER2-TGFbRII, and anti-nectin-4-TGFbRII. Further exemplary embodiments of this agent that localize to tumor-infiltrating T cells (e.g., tumor-infiltrating Tregs) include anti-CD39-TGFbRII, anti-CD73-TGFbRII, anti-CTLA4-TGFbRII. In a further embodiment, the localizing polypeptide of the TGFbRII-containing fusion protein is a receptor ECD. Exempary embodiments of this fusion protein include TGFbRII-Fc-SIRPa, TGFbRII-Fc-BTLA, TGFbRII-Fc-PD1, TGFbRII-Fc-TIM3. In other embodiments, tumor localization of TGFb inhibition is achieved by an ALT of the invention comprising anti-TGFb/TGFbR/GARP/LAP mAb fused to a receptor ECD that binds a tumor cell or tumor-infiltrating immune cell. Exemplary embodiments of this agent include anti-TGFb-SIRPa, anti-TGFb-SIGLEC10, anti-TGFb-BTLA, anti-TGFb-TIM3, anti-TGFb-PD1.

In some embodiments, the VEGF inhibitor is selected from the following: anti-VEGF antibody (e.g., bevacizumab), VEGFR antibody (e.g. ramucirumab), VEGFR kinase inhibitor (e.g., sunitinib, sorafenib, axitinib, cabozantinib, regorafenib, pazopanib, vandetanib, lenvatenib), or VEGFRecd-Fc fusion protein (e.g., aflibercept), or ALT comprising a ligand-binding sequence of VEGFRecd.

In some embodiments, the TGFb inhibitor is selected from the following: TGFbRI kinase inhibitor (e.g., galunisertib), anti-TGFb antibody (e.g., fresolimumab), anti-GARP antibody, anti-LAP antibody, anti-TGFbR antibody, fusion protein comprising TGFbRecd (e.g., TGFbRII-Fc), ALT comprising TGFbRecd (e.g., anti-PDL1-TGFbRIIecd, M7824, bintrafusp alfa, anti-CD73-TGFbRII, anti-CD39-TGFbRII).

The data in Example 5 demonstrate that tumor-targeted TGFbRII is effective in treating cancer. As such, in some embodiments of the invention, the fusion protein comprises TGFbRII ECD and a polypeptide that localizes the fusion protein to the tumor microenvironment. Exemplary embodiments of this fusion protein include anti-EGFRvIII-TGFbRII, anti-PSMA-TGFbRII, anti-nectin-4-TGFbRII, anti-CD47-TGFbRII.

Furthermore, Example 5 demonstrates that fusion proteins comprising TGFbRII and a polypeptide that induces or promotes ADCC/FcR-mediated cross-presentation is effective in treating cancer. As such, in some embodiments of the invention, the fusion protein comprises TGFbRII and an antibody that induces or promotes ADCC/FcR-mediated cross-presentation. In some embodiments, this fusion protein promotes ADCC/FcR-mediated cross-presentation by disabling a “don't eat me” signal on the tumor cell. In some embodiments, this “don't eat me” signal is CD47/SIRPa, SIGLEC10/CD24, CD31/CD31, or LILRB1/MHC. Exemplary embodiments of this fusion protein include anti-CD47-TGFbRII and SIRPa-Fc-TGFbRII and SIGLEC10-Fc-TGFbRII.

Furthermore, the invention comprises methods of treatment of cancer comprising one agent that is a TGFbRII-comprising fusion protein and another agent that promotes ADCC/FcR-mediated cross-presentation. In some embodiments, the TGFbRII-comprising fusion protein is an ALT or ECD-ECD comprising TGFbRII. In a particular aspect, the TGFbRII-comprising fusion protein comprises an antibody that binds a tumor cell surface molecule or tumor-infiltrating T cell cell surface molecule. Exemplary TGFbRII-comprising fusion proteins that bind tumor cell surface molecules include anti-EGFR-TGFbRII, anti-HER2-TGFbRII, anti-PSMA-TGFbRII, anti-nectin-4-TGFbRII, anti-IL17R-TGFbRII, and anti-PDL1-TGFbRII. Exemplary TGFbRII-comprising fusion proteins that bind tumor-infiltrating T cell cell surface molecules include anti-CD73-TGFbRII, anti-CD39-TGFbRII, anti-CTLA4-TGFbRII. Exemplary agents that promote ADCC/FcR-mediated cross presentation include anti-CD47, SIRPa-Fc, ALTs comprising anti-CD47, and ALTs comprising SIRPa ECD. Exemplary embodiments of this method of treatment include combination of anti-CD47 mAb with anti-CD73-TGFbRII, anti-CD47 mAb with anti-CD39-TGFbRII, anti-CD47 with anti-PDL1-TGFbRII, or anti-CD47 with anti-CTLA4-TGFbRII.

Furthermore, the invention comprises methods of treatment of cancer comprising one agent that blocks TGFb in the tumor microenvironment, and another agent that promotes ADCC/FcR-mediated cross-presentation. In some embodiments, the agent that blocks TGFb in the tumor microenvironment comprises an antibody to TGFb, TGFbR, LAP, or GARP fused to a receptor ECD that binds a tumor cell surface molecule or tumor-infiltrating T cell cell surface molecule (e.g., SIRPa ECD, BTLA ECD, TIM-3 ECD, PD-1 ECD, SIGLEC10 ECD).

In another aspect, fusion proteins of the invention counteract ITIM/ITSM signaling in the TME. In one embodiment, the fusion protein of the invention counteracts an immune cell inhibitory molecule that inhibits immune cell signaling, TCR signaling, T cell activation, macrophage phagocytosis, or dendritic cell antigen cross-presentation. In one embodiment, the immune cell inhibitor molecule exerts its inhibitor function via ITIMs or ITSMs. In one embodiment, the molecule of the invention comprises a ligand-binding sequence of the extracellular domain of a T cell co-inhibitory molecule (and is devoid of the transmembrane and intracellular domains containing ITIM or ITSM). In one aspect, the ligand-binding sequence of the extracellular domain of the T cell co-inhibitory molecule serves as a decoy or ligand-trap that binds its cognate ligand(s), thereby preventing ITIM/ITSM signaling by inhibiting the interaction of the ligand with the co-inhibitory molecule on the immune cell. In various examples, the molecule contains a ligand-binding sequence of the extracellular domain of PD-1 (PD1 ECD). In various examples, the molecule contains a ligand-binding sequence of the extracellular domain of TIM3 (TIM3 ECD). In various examples, the molecule contains a ligand-binding sequence of the extracellular domain of BTLA (BTLA ECD). In another aspect, the molecule of the invention comprises a ligand-binding sequence of the extracellular domain of an immune inhibitory molecule that exerts its inhibitory function via ITIMs and/or ITSMs. In various examples, the molecule contains a ligand-binding sequence of the extracellular domain of SIRPa (SIRPa ECD). In various examples, the molecule contains a ligand-binding sequence of the extracellular domain of SIGLEC10 (SIGLEC10 ECD).

In another embodiment, the molecule of the invention comprises a targeting polypeptide that binds either the immune cell inhibitory receptor or ligand to prevent the interaction leading to ITIM/ITSM-mediated inhibition, fused to one or more receptor ECDs. In one embodiment, the targeting polypeptide is an antibody. In various examples, the targeting polypeptide binds and disables CD24 or SIGLEC10; PD-1 or PD-L1; SIRPa or CD47; TIM-3 or a CEACAM family member that binds TIM-3; BTLA or HVEM. In various examples, the targeting polypeptide is an antibody that binds and disables CD24, SIGLEC10, PD-1, PD-L1, SIRPa, CD47, TIM-3, a CEACAM family member, BTLA, or HVEM.

As shown in Example 2, antibody-ligand traps containing BTLAecd localize to HVEM-expressing cells and simultaneously counteract BTLA-mediated suppression & promote HVEM-mediated activation of T cells. BTLA ligation by HVEM inhibits T cell activation via SHP-1-mediated inhibition of CD28 and CD3z signaling. HVEM ligation by LIGHT or BTLA (in trans) promotes T cell activation. The antibody ligand traps of the invention comprising a BTLA ecd which binds HVEM, thereby disrupting its interaction with both BTLA and CD160. In addition, ligation of T cell HVEM by BTLAecd of the ALT may promote HVEM-mediated costimulatory signals for T cell activation. PD-1 ligation by PD-1 ligands (PD-L1 or PD-L2) inhibits T cell activation via SHP-2-mediated inhibition of CD28 signaling. The interaction of PD-L1 with PD-1 can be disrupted by antibodies targeting either PD-L1 or PD-1, or a PD1 ecd that binds both PD-L1 and PD-L2. Antibody ligand traps comprising a BTLA ecd fused to an antibody that specifically binds PD-L1, or PD-1 can simultaneously inhibit PD-L1/PD-1 and HVEM/BTLA induced SHP1/2 mediated suppression of CD28 and CD3 signaling. As such, these molecules of the invention can counteract both HVEM/BTLA and PD-L1 mediated immune suppression in the tumor environment, thereby enhancing antitumor immune responses.

The data in Example 2 demonstrate that BTLA ECD fused to the heavy or light chain of an antibody is capable of binding HVEM, thereby disrupting native BTLA-mediated SHP1/SHP2 inhibition, and promoting HVEM-mediated co-stimulatory signaling. As such, in some embodiments, the fusion proteins of the invention comprise an antibody and BTLA ECD where BTLA ECD is fused to either light chain or heavy chain of antibody. In one aspect, the BTLAecd is fused to the heavy chain of an antibody, with or without a linker. In a further aspect, BTLAecd is fused to the C terminus of immunoglobulin Fc, with or without a linker. In one aspect, BTLAecd is fused to the light chain of an antibody, with or without a linker. In a further aspect, BTLAecd is fused to the C terminus of the light chain, with or without a linker.

Furthermore, Example 2 demonstrates that inhibition of BTLA/HVEM signaling with a decoy BTLA receptor ECD fused to a polypeptide that binds and disables another immuno-inhibitory molecule is effective in the treatment of cancer. As such, in some embodiments, the fusion proteins of the invention comprise a BTLA ECD and a targeting polypeptide that specifically binds an immune cell inhibitory molecule that inhibits the function of T cells, macrophages and/or dendritic cells. In one aspect, the immune cell inhibitory molecule has an intracellular domain comprising ITIM or ITSM motifs. In one aspect, the inhibitory molecule is a ligand that binds an inhibitory receptor containing ITIM or ITSM motifs. In one aspect, the inhibitory receptor signals via SHP1 or SHP2. In one aspect, the targeting polypeptide inhibits the function of the immune cell inhibitory molecule as an antagonist. In one aspect, the targeting polypeptide is an antibody. In another aspect, the targeting polypeptide is a Fc fusion protein. Examples of the immuno-inhibitory molecules include, but are not limited to the following: CD47, SIRPa, CD24, SIGLEC-10, LILRB, PD-L1, PD-L2, PD1, TIGIT, PVRIG, TIM-3, CEACAM1, CEACAM5.

Furthermore, Example 2 demonstrates that the decoy BTLA receptor ECD fused to a polypeptide that binds and disables another T cell co-inhibitory molecule is effective in the treatment of cancer. As such, in some fusion proteins, BTLA ECD is fused to a targeting polypeptide that specifically binds a T cell co-inhibitory molecule. In one aspect, the targeting polypeptide inhibits the function of the T cell co-inhibitory molecule. In one aspect, the targeting polypeptide is an antibody. In another aspect, the targeting polypeptide is a Fc fusion protein. Examples of the T cell co-inhibitory molecule include, but are not limited to the following: PD-L1, PD-L2, PD1, CTLA-4, TIGIT, PVRIG, TIM-3, TIM-3 ligand, CEACAM1, CEACAM5, VISTA, VSIG8.

Furthermore, Example 2 demonstrates that decoy BTLA receptor ECD fused to a polypeptide that inhibits the interaction of a cytokine and its cytokine receptor is effective in the treatment of cancer. As such, in some embodiments of the invention, BTLA ECD is fused to a targeting polypeptide that specifically binds a cytokine or cytokine receptor. In one aspect, the cytokine or cytokine receptor inhibit the function of T cells, macrophages, and/or dendritic cells. In one aspect, the cytokine or cytokine receptor promote tumor angiogenesis. In one aspect, the targeting polypeptide is an antibody. In another aspect, the targeting polypeptide is a ligand-binding sequence of a cytokine receptor extracellular domain. Examples of the cytokine/cytokine receptor include TGFb/TGFbR, IL-17/IL-17R, IL-23/IL-23R, IL-6/IL-6R, IL-1/IL-1R, IL-10/IL-10R, and VEGF/VEGFR.

Furthermore, Example 2 demonstrates that decoy BTLA receptor ECD fused to a polypeptide that binds a tumor cell surface molecule is effective in the treatment of cancer. As such, in some embodiments of the invention, BTLA ECD is fused to a polypeptide that binds a tumor cell surface molecule. In some embodiments, this tumor cell-surface molecule is a growth factor or growth factor receptor. In other embodiments, this tumor cell surface molecule is a protein that is overexpressed on tumor cells. Examples of the tumor cell surface molecule include PD-L1, EGFR, HER2, EGFRvIII, PSMA, nectin-4, and uPAR. In a particular aspect, the targeting polypeptide is a bispecific antibody. In a further aspect, the bispecific antibody is a CrossMab or a BiTE. In a further aspect, the bispecific antibody binds CD3 and a tumor cell surface molecule. Examples of the bispecific antibody include CD3×HER2 bsAbs and CD3×CEA bsAbs.

Furthermore, Example 2 demonstrates that decoy BTLA receptor ECD on either the heavy or light chain of the targeting polypeptide is capable of binding HVEM, thereby disrupting native BTLA-mediated SHP1/SHP2 inhibition and promoting HVEM-mediated co-stimulatory signaling, even while another receptor ECD is additionally fused to the antibody. Furthermore, these data demonstrate that decoy BTLA receptor ECD is effective in the treatment of cancer when part of a fusion protein comprising an additional ECD of a cytokine or cytokine receptor. As such, in some embodiments of the invention, the fusion protein comprising BTLA ECD further comprises an additional ECD (ECD #2) selected from: TGFbRII ECD, VEGFR ECD, SIRPa ECD, SIGLEC10 ECD, ECD of a T cell co-inhibitory molecule (e.g., TIM3 ECD, or PD1 ECD). In other embodiments, the fusion protein comprising BTLA ECD further comprises an additional ECD (ECD #2) of a cytokine. In some embodiments, this cytokine is IL-15 or IL-12. In some embodiments, BTLA ECD is fused to the heavy chain or light chain of the targeting antibody and ECD #2 is fused to the heavy or light chain. In some embodiments, ECD #2 is fused to the heavy chain. In other embodiments, ECD #2 is fused to the light chain. In some embodiments, ECD #2 is TGFbRII ECD. In other embodiments, ECD #2 is a ligand-binding fragment of a T cell co-inhibitory molecule. In some embodiments, ECD #2 is TIM3 ECD. In other embodiments, ECD #2 is VEGFR ECD. In other embodiments, ECD #2 is a cytokine receptor ECD. In other embodiments, ECD #2 is the ECD of a receptor whose ligation inhibits phagocytosis. In some embodiments, ECD #2 is SIRPa ECD or SIGLEC10 ECD. In other embodiments, BTLA ECD and ECD #2 are fused together, with or without a linker; with or without an Fc domain between them.

Furthermore, Example 2 demonstrates that decoy BTLA receptor ECD fused to an antibody can enable recruitment of T cells to tumor cells, since these data show that BTLA ECD can bind HVEM while the targeting antibody simultaneously binds a T cell surface molecule. As such, in some embodiments of the invention, BTLA ECD is fused to an antibody that binds T cells. In some embodiments, the antibody binds CD3. In a particular aspect, the antibody is a bispecific antibody. In a further aspect, the antibody is a bispecific antibody that binds CD3 and another target.

In various embodiments, the fusion proteins of the invention counteract PD-1/PD-L1 in the tumor microenvironment. In some embodiments, the fusion proteins comprise a ligand-binding sequence of an extracellular domain of PD-1 (e.g., PD1 ECD).

In one embodiment, the fusion protein of the invention comprises an antibody that binds PD1 or PD-1 ligand. In one example, the ALT comprises an antibody that binds PD1 and interferes with its interaction with PD-1 ligand. In one example, the ALT comprises an antibody that binds PDL1 and interferes with its interaction with PD-1 or B7. In one aspect, the antibody is an antagonist that inhibits PD1/PD1 ligand interaction or intracellular ITIM or ITSM signaling, thereby promoting immune cell activation. In various examples, the antibody that binds PD1 or PD1 ligand is fused to one or more ligand traps. In one example the PD1 or PDL1 antibody is fused to a ligand-binding sequence of the extracellular domain of TIM3 (TIM3 ECD). In one example the PD1 or PDL1 antibody is fused to a ligand-binding sequence of the extracellular domain of TGFbR (TGFbRII ECD). In one example the PD1 or PDL1 antibody is fused to a ligand-binding sequence of the extracellular domain of BTLA (BTLA ECD). In one example the PD1 or PDL1 antibody is fused to a ligand-binding sequence of the extracellular domain of SIRPa (SIRPa ECD). In one example the PD1 or PDL1 antibody is fused to a ligand-binding sequence of the extracellular domain of VEGFR (VEGFR ECD). In one embodiment, the PD1 or PDL1 binding antibody is fused to multiple ligand traps selected from the following: TIM3 ECD, TGFbRII ECD, BTLA ECD, SIRPa ECD, VEGFR ECD. In one aspect, a ligand trap is fused to the heavy chain, and a second ligand trap is fused to the light chain.

In various examples, the ALT comprises a PD1 or PDL1 binding antibody, wherein the heavy chain is fused to TGFbRII ECD and the light chain is fused to TIM3 ECD or BTLA ECD or SIRPa ECD or SIGLEC10 ECD. In various examples, the ALT comprises a PD1 or PDL1 binding antibody, wherein the heavy chain is fused to TIM3 ECD and the light chain is fused to BTLA ECD or SIRPa ECD or SIGLEC10 ECD. In various examples, the ALT comprises a PD1 or PDL1 binding antibody, wherein the heavy chain is fused to BTLA ECD and the light chain is fused to TIM3 ECD or SIGLEC10 ECD or SIRPa ECD.

In various examples, the ALT comprises a PD1 or PDL1 binding antibody, wherein the heavy chain is fused to VEGFR ECD and the light chain is fused to TIM3 ECD, SIGLEC10 ECD, SIRPa ECD, or BTLA ECD.

In one embodiment, the fusion proteins of the invention may comprise a ligand-binding sequence of an extracellular domain of Programmed-Death 1 (PD1). In one aspect, the PD1 ECD binds and disables PD-L1 or PD-L2. In one aspect, the PD1 ECD interferes with the interaction of PD1 ligands with either PD1 or B7.

In some embodiments, the fusion protein of the invention comprises a targeting polypeptide that is an antibody. In one embodiment, the antibody is fused to PD1 ECD and additional ligand traps selected from the following: TIM3 ECD, TGFbRII ECD, BTLA ECD, SIRPa ECD, VEGFR ECD, SIGLEC10 ECD. In one aspect, a ligand trap (LT1) is fused to the heavy chain, and a second ligand trap (LT2) is fused to the light chain. In one aspect, the PD1 ECD is fused to the heavy chain and another ligand trap is fused to the light chain. In one aspect, the PD1 ECD is fused to the light chain and another ligand trap is fused to the heavy chain. In various examples, the ALT comprises an antibody, wherein the heavy chain is fused to PD1 ECD and the light chain is fused to one of TIM3 ECD, BTLA ECD, SIRPa ECD, or SIGLEC10 ECD. In various examples, the ALT comprises an antibody wherein the light chain is fused to PD1 ECD and the heavy chain is fused to one of TIM3 ECD, TGFbRII ECD, BTLA ECD, SIRPa ECD, VEGFR ECD, SIGLEC10 ECD.

In some embodiments, the fusion protein comprises PD1 ECD and a polypeptide that inhibits the interaction between CD47 and SIRPa. In one embodiment, this fusion protein comprises PD1 ECD and a polypeptide that binds CD47. In one embodiment, this fusion protein is anti-CD47 mAb fused to PD1 ECD (anti-CD47-PD1 (e.g., SEQ ID NOs: 387, 22)). In another embodiment, this fusion protein comprises SIRPa ECD and PD1 ECD. In one embodiment, this fusion protein is SIRPa-Fc-PD1 (e.g., SEQ ID NO: 548) or PD1-Fc-SIRPa (e.g., SEQ ID NO: 537).

In a further aspect, the fusion protein comprises PD1 ECD, a polypeptide that inhibits the interaction between CD47 and SIRPa, and another polypeptide that inhibits a T cell co-inhibitory molecule. In some embodiments, this fusion protein comprises anti-CD47 mAb with PD1 ECD fused to the heavy chain or light chain; and a T cell co-inhibitory molecule ECD fused to the other chain.

In another aspect, the fusion protein comprises PD1 ECD, SIRPa ECD, and an antibody with a heavy chain and light chain. In one aspect, the PD1 ECD is fused to the heavy chain of the antibody and the SIRPa ECD is fused to the light chain of the antibody. In another aspect, the PD1 ECD is fused to the light chain of the antibody and the SIRPa ECD is fused to the heavy chain of the antibody. In some embodiments, the antibody of said fusion protein binds a T cell co-inhibitory molecule as an antagonist. An exemplary embodiment of such a fusion protein includes anti-CTLA4-PD1-SIRPa (e.g., SEQ ID NOs: 440, 438). In other embodiments, the antibody of said fusion protein binds a T cell co-stimulatory molecule as an agonist. Exemplary embodiments of this fusion protein include anti-OX40-PD1-SIRPa (e.g., SEQ ID NOs: 510, 508), anti-41BB-PD1-SIRPa (e.g., SEQ ID NOs: 498, 496), and anti-CD40-PD1-SIRPa. In other embodiments, the antibody of said fusion protein is a tumor-targeted antibody. In some embodiments, this tumor targeted-antibody binds a tumor growth factor or growth factor receptor. In other embodiments, this tumor targeted antibody binds a tumor cell surface molecule. Exemplary embodiments of this fusion protein include anti-EGFR-PD1-SIRPa (e.g., SEQ ID NOs: 225, 223), anti-HER2-PD1-SIRPa (e.g., SEQ ID NOs: 249, 247), anti-EGFRvIII-PD1-SIRPa (e.g., SEQ ID NOs: 237, 235), anti-uPAR-PD1-SIRPa, and anti-PSMA-PD1-SIRPa.

In other embodiments, the antibody of said fusion protein binds a member of the TGFb pathway. In some embodiments, this antibody binds TGFb, TGFbR, or GARP. Exemplary embodiments of this fusion protein include anti-TGFb-PD1-SIRPa (e.g., SEQ ID NOs: 398, 396), anti-TGFbR-PD1-SIRPa, and anti-GARP-PD1-SIRPa.

In other embodiments, the antibody of said fusion protein binds VEGF or VEGFR. Exemplary embodiments of this fusion protein include anti-VEGF-PD1-SIRPa (e.g., SEQ ID NOs: 366, 364) and anti-VEGFR-PD1-SIRPa (e.g., SEQ ID NOs: 377, 375).

In some embodiments, the fusion protein comprises PD1 ECD and a polypeptide that inhibits a T cell co-inhibitory molecule. In specific embodiments, the fusion protein comprises PD1 ECD and an antibody that binds and disables a T cell co-inhibitory molecule. In various examples, the T cell co-inhibitory molecule is CTLA-4, LAG3, TIM-3, CEACAM, CD47, SIRPa, TIGIT, VISTA, VSIG8, PVRIG, or BTLA.

In some embodiments, the fusion protein comprises PD1 ECD and a polypeptide that inhibits the interaction of TIGIT or PVRIG with PVRL2 or PVR. In some embodiments, this fusion protein comprises an antibody that binds TIGIT or PVRIG fused to PD1 ECD. Exemplary embodiments of this fusion protein include anti-TIGIT-PD1 (e.g., SEQ ID NOs: 474, 139) and anti-PVRIG-PD1.

In some embodiments, the fusion protein comprises PD1 ECD and a polypeptide that inhibits the interaction of VISTA and VSIG8. In some embodiments, this fusion protein comprises an antibody that binds VISTA or VSIG8 fused to PD1 ECD. Exemplary embodiments of this fusion protein include anti-VISTA-PD1 and anti-VSIG8-PD1.

In some embodiments, the fusion protein comprises PD1 ECD, an antibody that binds and disables a T cell co-inhibitory molecule, and an additional receptor ECD. In one aspect, the PD1 ECD is fused to the heavy chain of the antibody and the additional receptor ECD is fused to the light chain of the antibody. Alternatively, the PD1 ECD is fused to the light chain and the additional receptor ECD is fused to the heavy chain.

In other embodiments, the fusion protein comprises PD1 ECD, an antibody that binds and disables a T cell co-inhibitory molecule, and SIRPa ECD. Exemplary embodiments of this fusion protein include anti-PD1-PD1-SIRPa (e.g., SEQ ID NOs: 452, 450); anti-CTLA4-PD1-SIRPa (e.g., SEQ ID NOs: 440, 438); anti-TIGIT-PD1-SIRPa (e.g., SEQ ID NOs: 474, 472); anti-TIM3-PD1-SIRPa (e.g., SEQ ID NOs: 486, 484).

In other embodiments, the fusion protein comprises PD1 ECD, an antibody that binds and disables a T cell co-inhibitory molecule, and SIGLEC10 ECD. Exemplary embodiments of this fusion protein include anti-TIGIT-PD1-SIGLEC10 (e.g., SEQ ID NOs: 474, 471); anti-PD1-PD1-SIGLEC10 (e.g., SEQ ID NOs: 452, 449); anti-CTLA4-PD1-SIGLEC10 (e.g., SEQ ID NOs: 440, 437); anti-TIM3-PD1-SIGLEC10 (e.g., SEQ ID NOs: 486, 483).

In some embodiments, the fusion protein comprises an antibody that binds a T cell co-stimulatory molecule fused to PD1. This antibody is preferably an agonist of the T cell co-stimulatory molecule. In some embodiments, this fusion protein is selected from: anti-ICOS-PD1 (e.g., SEQ ID NOs: 522, 59); anti-41BB-PD1 (e.g., SEQ ID NOs: 498, 2); anti-OX40-PD1 (e.g., SEQ ID NOs: 510, 97).

In a further aspect, the fusion protein comprises an antibody that binds a T cell-costimulatory molecule, PD1 ECD, and an additional receptor ECD. In one aspect, the PD1 ECD is fused to the heavy chain of the antibody and the additional receptor ECD is fused to the light chain of the antibody. Alternatively, the PD1 ECD is fused to the light chain of the antibody and the additional receptor ECD is fused to the heavy chain of the antibody. In some embodiments, the additional receptor ECD is the ECD of a T cell co-inhibitory molecule. In another aspect, the additional receptor ECD is SIRPa ECD. In some embodiments, the fusion protein is selected from: anti-ICOS-PD1-SIRPa (e.g., SEQ ID NOs: 522, 520); anti-OX40-PD1-SIRPa (e.g., SEQ ID NOs: 510, 508); anti-41BB-PD1-SIRPa (e.g., SEQ ID NOs: 498, 496). In another aspect, the additional receptor ECD is SIGLEC10 ECD. In some embodiments, the fusion protein is selected from: anti-OX40-PD1-SIGLEC10 (e.g., SEQ ID NOs: 510, 507); anti-ICOS-PD1-SIGLEC10 (e.g., SEQ ID NOs: 522, 519); anti-41BB-PD1-SIGLEC10 (e.g., SEQ ID NOs: 498, 495).

In some embodiments, the fusion protein comprises an antibody, PD1 ECD, and the ECD of a T cell co-stimulatory molecule. In one aspect, the PD1 ECD is fused to heavy chain and the ECD of a T cell co-stimulatory molecule fused to light chain. Alternatively, the PD1 ECD is fused to the light chain and the T cell co-stimulatory molecule ECD is fused to the heavy chain. In some embodiments, the fusion protein comprises PD1 ECD and one of the following: OX40L, 41BBL, ICOSL.

In one embodiment, the fusion protein comprises PD1 ECD and a polypeptide that binds an ectonucleotidase. In a preferred embodiment, the ectonucleotidase is either CD39 or CD73. In some embodiments, the fusion protein is an antibody that binds CD39 or CD73 fused to PD1 ECD; for example: anti-CD39-PD1 (e.g., SEQ ID NOs: 429, 18) or anti-CD73-PD1 (e.g., SEQ ID NOs: 421, 24).

In some embodiments, the fusion protein comprises PD1 ECD, an antibody that binds CD39 or CD73, and an additional receptor ECD. In some embodiments, the PD1 ECD is fused to the heavy chain of the antibody and the additional receptor ECD is fused to the light chain of the antibody. Alternatively, the PD1 ECD is fused to the light chain of the antibody and the additional receptor ECD is fused to the heavy chain of the antibody. In some embodiments, the fusion protein is selected from the following: anti-CD39-PD1-SIRPa, or anti-CD73-PD1-SIRPa (e.g., SEQ ID NOs: 421, 419).

In some embodiments, the fusion protein comprises a tumor-targeted antibody and PD1 ECD. In some embodiments, this tumor targeted-antibody binds a tumor growth factor or growth factor receptor. In other embodiments, this tumor targeted antibody binds a tumor cell surface molecule. In some embodiments, this fusion protein is selected from the following: anti-EGFR-PD1 (e.g., SEQ ID NOs: 225, 43), anti-HER2-PD1 (e.g., SEQ ID NOs: 249, 55), anti-EGFRvIII-PD1 (e.g., SEQ ID NOs: 237, 47), anti-uPAR-PD1 (e.g., SEQ ID NOs: 269, 162), anti-PSMA-PD1 (e.g., SEQ ID NOs: 276, 121).

In a further embodiment, the fusion protein comprises a tumor-targeted antibody, PD1 ECD, and an additional receptor ECD. In one embodiment, the PD1 ECD is fused to the heavy chain of the antibody and the additional receptor ECD is fused to the light chain of the antibody. Alternatively, the PD1 ECD is fused to the light chain and the additional receptor ECD is fused to the heavy chain. In some embodiments, the additional receptor ECD is the ECD of a T cell co-inhibitory molecule. In another aspect, the additional receptor ECD is SIRPa ECD. In some embodiments, the fusion protein is selected from: anti-EGFR-PD1-SIRPa (e.g., SEQ ID NOs: 225, 223); anti-HER2-PD1-SIRPa (e.g., SEQ ID NOs: 249, 247); anti-EGFRvIII-PD1-SIRPa (e.g., SEQ ID NOs: 237, 235); anti-nectin4-PD1-SIRPa (e.g., SEQ ID NOs: 261, 259). In another aspect, the additional receptor ECD is SIGLEC10 ECD. In some embodiments, the fusion protein is selected from: anti-EGFR-PD1-SIGLEC10 (e.g., SEQ ID NOs: 225, 222); anti-nectin4-PD1-SIGLEC10 (e.g., SEQ ID NOs: 261, 258); anti-HER2-PD1-SIGLEC10 (e.g., SEQ ID NOs: 249, 246); anti-EGFRvIII-PD1-SIGLEC10 (e.g., SEQ ID NOs: 237, 234).

In some embodiments, the fusion protein comprises PD1 ECD and a polypeptide that inhibits VEGF/VEGFR signaling. In some embodiments, the fusion protein comprises PD1 ECD and anti-VEGFR mAb with PD1 ECD fused to either the heavy chain or light chain of the antibody. In some embodiments, this fusion protein is anti-VEGFR-PD1 (e.g., SEQ ID NOs: 377, 148). In a further embodiment, the fusion protein may additionally comprise a receptor ECD fused to the other chain of the antibody. In some embodiments, the fusion protein is selected from anti-VEGFR-PD1-TIM3 (e.g., SEQ ID NOs: 377, 376); anti-VEGFR-PD1-BTLA (e.g., SEQ ID NOs: 377, 378); anti-VEGFR-PD1-SIRPa (e.g., SEQ ID NOs: 377, 375); anti-VEGFR-PD1-SIGLEC10 (e.g., SEQ ID NOs: 377, 374).

In some embodiments, the fusion protein comprises PD1 ECD and anti-VEGF mAb with PD1 ECD fused to either the heavy chain or light chain of the antibody. In some embodiments, this fusion protein is anti-VEGF-PD1 (e.g., SEQ ID NOs: 366, 32). In a further embodiment, the fusion protein may additionally comprise a receptor ECD fused to the other chain of the antibody. In some embodiments, the fusion protein is selected from anti-VEGF-PD1-BTLA (e.g., SEQ ID NOs: 366, 367); anti-VEGF-PD1-TIM3 (e.g., SEQ ID NOs: 366, 365); anti-VEGF-PD1-SIRPa (e.g., SEQ ID NOs: 366, 364); anti-VEGF-PD1-SIGLEC10 (e.g., SEQ ID NOs: 366, 363).

In other embodiments, the fusion protein comprises PD1 ECD and VEGFR ECD. In one embodiment, this fusion protein is PD1-Fc-VEGFR (e.g., SEQ ID NO: 540). In another embodiment, this fusion protein is VEGFR-Fc-PD1 (e.g., SEQ ID NO: 566)

In some embodiments, the fusion protein comprises an antibody that binds IL-17 or IL-17R and PD1 ECD. In some embodiments, this fusion protein is selected from the following: anti-IL17-PD1, anti-IL17R-PD1 (e.g., SEQ ID NOs: 330, 63).

In a further embodiment, the fusion protein comprises an antibody that binds IL-17 or IL-17R, PD1 ECD, and an additional receptor ECD. In one embodiment, PD1 ECD is fused to the heavy chain of the antibody and the additional receptor ECD is fused to the light chain of the antibody. Alternatively, PD1 ECD is fused to light chain and additional receptor ECD is fused to heavy chain. In some embodiments, the additional receptor ECD is the ECD of a T cell co-inhibitory molecule. In another aspect, the additional receptor ECD is SIRPa ECD. In some embodiments, the fusion protein is selected from: anti-IL17R-PD1-SIRPa (e.g., SEQ ID NOs: 330, 328). In another aspect, the additional receptor ECD is SIGLEC10 ECD. In some embodiments, the fusion protein is selected from: anti-IL17R-PD1-SIGLEC10 (e.g., SEQ ID NOs: 330, 327).

In some embodiments, the fusion protein comprises an antibody that binds IL-23 or IL-23R and PD1 ECD. If the antibody binds IL-23, it is preferred that the antibody bind the p19 subunit of IL-23 that is not shared with IL-12. In some embodiments, this fusion protein is selected from the following: anti-IL23-PD1 (e.g., SEQ ID NOs: 342, 75), anti-IL23R-PD1.

In a further embodiment, the fusion protein comprises an antibody that binds IL-23 or IL-23R, PD1 ECD, and an additional receptor ECD. In one embodiment, PD1 ECD is fused to the heavy chain of the antibody and the additional receptor ECD is fused to the light chain of the antibody. Alternatively, PD1 ECD is fused to light chain and additional receptor ECD is fused to heavy chain. In some embodiments, the additional receptor ECD is the ECD of a T cell co-inhibitory molecule. In another aspect, the additional receptor ECD is SIRPa ECD. In some embodiments, the fusion protein is selected from: anti-IL23-PD1-SIRPa (e.g., SEQ ID NOs: 342, 340). In another aspect, the additional receptor ECD is SIGLEC10 ECD. In some embodiments, the fusion protein is selected from: anti-IL23-PD1-SIGLEC10 (e.g., SEQ ID NOs: 342, 339).

In some embodiments, the fusion protein comprises an antibody that binds IL-6 or IL-6R, PD1 ECD, and an additional receptor ECD. In one embodiment, PD1 ECD is fused to the heavy chain of the antibody and the additional receptor ECD is fused to the light chain of the antibody. Alternatively, PD1 ECD is fused to light chain and additional receptor ECD is fused to heavy chain. In some embodiments, the additional receptor ECD is the ECD of a T cell co-inhibitory molecule. In another aspect, the additional receptor ECD is SIRPa ECD. In some embodiments, the fusion protein is selected from: anti-IL6R-PD1-SIRPa (e.g., SEQ ID NOs: 318, 316). In another aspect, the additional receptor ECD is SIGLEC10 ECD. In some embodiments, the fusion protein is selected from: anti-IL6R-PD1-SIGLEC10 (e.g., SEQ ID NOs: 318, 315).

In some embodiments, the fusion protein comprises PD1 ECD and an antibody that binds TGFb, TGFbR, or GARP. Exemplary embodiments of this fusion protein include anti-TGFb-PD1 (e.g., SEQ ID NOs: 398, 133), anti-TGFbR-PD1, and anti-GARP-PD1 (e.g., SEQ ID NOs: 411, 49).

In some embodiments, the fusion protein further comprises an additional receptor ECD. Exemplary embodiments of this fusion protein include anti-TGFb-PD1-SIRPa (e.g., SEQ ID NOs: 398, 396); anti-TGFb-PD1-BTLA (e.g., SEQ ID NOs: 398, 399); anti-TGFb-PD1-SIGLEC10 (e.g., SEQ ID NOs: 398, 395); anti-TGFb-PD1-TIM3 (e.g., SEQ ID NOs: 398, 397).

In some embodiments, the fusion protein comprises PD1 ECD and IL-15. In some embodiments, the fusion protein is IL15-Fc-PD1 (e.g., SEQ ID NO: 578) or PD1-Fc-IL15 (e.g., SEQ ID NO: 577). In other embodiments, the fusion protein is IL12-Fc-PD1 (e.g., SEQ ID NO: 576) or PD1-Fc-IL12 (e.g., SEQ ID NO: 575). In other embodiments, the fusion protein comprises an antibody with PD1 ECD fused to heavy chain and IL-15 fused to light chain. In other embodiments, the fusion protein comprises an antibody with PD1 ECD fused to heavy chain and IL-12 fused to light chain.

In various embodiments, the fusion proteins of the invention counteract TIM-3/CEACAM in the tumor microenvironment. In some embodiments, the fusion proteins comprise a ligand-binding sequence of an extracellular domain of TIM-3 (e.g., TIM-3 ECD).

In one embodiment, the fusion protein is an ALT that comprises an antibody that binds TIM3 or a TIM3 ligand (e.g. CEACAM1). In one example, the ALT comprises an antibody that binds TIM3 and interferes with its interaction with CEACAM1. In one example, the ALT comprises an antibody that binds CEACAM and interferes with its heterodimerization with TIM3 or homodimerization with CEACAM. In one aspect, the antibody is an antagonist that inhibits TIM3/TIM3 ligand interaction or intracellular ITIM or ITSM signaling, thereby promoting immune cell activation. In various examples, the antibody that binds TIM3 or CEACAM is fused to one or more ligand traps. In one example the TIM3 or CEACAM antibody is fused to a ligand-binding sequence of the extracellular domain of PD1 (PD1 ECD). In one example the TIM3 or CEACAM antibody is fused to a ligand-binding sequence of the extracellular domain of TGFbR (TGFbRII ECD). In one example the TIM3 or CEACAM antibody is fused to a ligand-binding sequence of the extracellular domain of BTLA (BTLA ECD). In one example the TIM3 or CEACAM antibody is fused to a ligand-binding sequence of the extracellular domain of SIRPa (SIRPa ECD). In one example the TIM3 or CEACAM antibody is fused to a ligand-binding sequence of the extracellular domain of VEGFR (VEGFR ECD). In one embodiment, the TIM3 or CEACAM binding antibody is fused to multiple ligand traps selected from the following: PD1 ECD, TGFbRII ECD, BTLA ECD, SIRPa ECD, VEGFR ECD, SIGLEC10 ECD. In one aspect, a ligand trap is fused to the heavy chain, and a second ligand trap is fused to the light chain.

In various examples, the ALT comprises a TIM3 or CEACAM binding antibody, wherein the heavy chain is fused to TGFbRII ECD and the light chain is fused to PD1 ECD, BTLA ECD, SIRPa ECD, or SIGLEC10 ECD. In various examples, the ALT comprises a TIM3 or CEACAM binding antibody, wherein the heavy chain is fused to VEGFR ECD and the light chain is fused to PD1 ECD, BTLA ECD, SIRPa ECD, or SIGLEC10 ECD. In various examples, the ALT comprises a TIM3 or CEACAM binding antibody, wherein the heavy chain is fused to PD1 ECD and the light chain is fused to BTLA ECD, SIRPa ECD, or SIGLEC10 ECD. In various examples, the ALT comprises a TIM3 or CEACAM binding antibody, wherein the heavy chain is fused to BTLA ECD and the light chain is fused to PD1 ECD, SIRPa ECD, or SIGLEC10 ECD. In various examples, the ALT comprises a TIM3 or CEACAM binding antibody, wherein the heavy chain is fused to SIRPa ECD and the light chain is fused to BTLA ECD, PD1 ECD, or SIGLEC10 ECD. In various examples, the ALT comprises a TIM3 or CEACAM binding antibody, wherein the heavy chain is fused to SIGLEC10 ECD and the light chain is fused to BTLA ECD, SIRPa ECD, or PD1 ECD.

In one embodiment, the fusion proteins of the invention comprise a ligand-binding sequence of an extracellular domain of TIM-3 (TIM3 ECD) to bind and disable TIM-3 ligands (CEACAM1, CEACAM5).

In some embodiments, the fusion protein of the invention comprises a targeting polypeptide that is an antibody. In one embodiment, the antibody is fused to TIM3 ECD and additional ligand traps selected from the following: PD1 ECD, TGFbRII ECD, BTLA ECD, SIRPa ECD, VEGFR ECD, SIGLEC10 ECD. In one aspect, a ligand trap (LT1) is fused to the heavy chain, and a second ligand trap (LT2) is fused to the light chain. In one aspect, the TIM3 ECD is fused to the heavy chain and another ligand trap is fused to the light chain. In one aspect, the TIM3 ECD is fused to the light chain and another ligand trap is fused to the heavy chain. In various examples, the ALT comprises an antibody, wherein the heavy chain is fused to TIM3 ECD and the light chain is fused to one of PD1 ECD, BTLA ECD, SIRPa ECD, or SIGLEC10 ECD. In various examples, the ALT comprises an antibody wherein the light chain is fused to TIM3 ECD and the heavy chain is fused to one of PD1 ECD, TGFbRII ECD, BTLA ECD, SIRPa ECD, VEGFR ECD, SIGLEC10 ECD.

In some embodiments, the fusion protein comprises TIM3 ECD and a polypeptide that inhibits the interaction between CD47 and SIRPa. In one embodiment, this fusion protein comprises TIM3 ECD and a polypeptide that binds CD47. In one embodiment, this fusion protein is anti-CD47 mAb fused to TIM3 ECD (anti-CD47-TIM3 (e.g., SEQ ID NOs: 391, 22)). In another embodiment, this fusion protein comprises SIRPa ECD and TIM3 ECD. In one embodiment, this fusion protein is SIRPa-Fc-TIM3 (e.g., SEQ ID NO: 551) or TIM3-Fc-SIRPa (e.g., SEQ ID NO: 562).

In a further aspect, the fusion protein comprises TIM3 ECD, a polypeptide that inhibits the interaction between CD47 and SIRPa, and another polypeptide that inhibits a T cell co-inhibitory molecule. In some embodiments, this fusion protein comprises anti-CD47 mAb with TIM3 ECD fused to the heavy chain or light chain; and a T cell co-inhibitory molecule ECD fused to the other chain. In a particular embodiment, this fusion protein is anti-CD47-TIM3-PD1 (e.g., SEQ ID NOs: 391, 384).

In another aspect, the fusion protein comprises TIM3 ECD, anti-CD47 mAb, and VEGFR ECD. In some embodiments, VEGFR ECD is fused to heavy chain of anti-CD47 mAb and TIM3 ECD is fused to light chain of anti-CD47 mAb. In one embodiment, this fusion protein is anti-CD47-VEGFR-TIM3 (e.g., SEQ ID NOs: 392, 386)

In another aspect, the fusion protein comprises TIM3 ECD, SIRPa ECD, and an antibody with a heavy chain and light chain. In one aspect, the TIM3 ECD is fused to the heavy chain of the antibody and the SIRPa ECD is fused to the light chain of the antibody. In another aspect, the TIM3 ECD is fused to the light chain of the antibody and the SIRPa ECD is fused to the heavy chain of the antibody. In some embodiments, the antibody of said fusion protein binds a T cell co-inhibitory molecule as an antagonist. Exemplary embodiments of such fusion proteins include anti-CTLA4-TIM3-SIRPa (e.g., SEQ ID NOs: 445, 438), anti-PD1-TIM3-SIRPa (e.g., SEQ ID NOs: 457, 450), and anti-PDL1-TIM3-SIRPa (e.g., SEQ ID NOs: 467, 461). In other embodiments, the antibody of said fusion protein binds a T cell co-stimulatory molecule as an agonist. Exemplary embodiments of this fusion protein include anti-OX40-TIM3-SIRPa (e.g., SEQ ID NOs: 515, 508), anti-41BB-TIM3-SIRPa (e.g., SEQ ID NOs: 503, 496), and anti-CD40-TIM3-SIRPa. In other embodiments, the antibody of said fusion protein is a tumor-targeted antibody. In some embodiments, this tumor targeted-antibody binds a tumor growth factor or growth factor receptor. In other embodiments, this tumor targeted antibody binds a tumor cell surface molecule. Exemplary embodiments of this fusion protein include anti-EGFR-TIM3-SIRPa (e.g., SEQ ID NOs: 230, 223), anti-HER2-TIM3-SIRPa (e.g., SEQ ID NOs: 254, 247), anti-EGFRvIII-TIM3-SIRPa (e.g., SEQ ID NOs: 242, 235), anti-uPAR-TIM3-SIRPa, and anti-PSMA-TIM3-SIRPa.

In other embodiments, the antibody of said fusion protein binds a member of the TGFb pathway. In some embodiments, this antibody binds TGFb, TGFbR, or GARP.

Exemplary embodiments of this fusion protein include anti-TGFb-TIM3-SIRPa (e.g., SEQ ID NOs: 402, 396), anti-TGFbR-TIM3-SIRPa, and anti-GARP-TIM3-SIRPa.

In other embodiments, the antibody of said fusion protein binds VEGF or VEGFR. Exemplary embodiments of this fusion protein include anti-VEGF-TIM3-SIRPa (e.g., SEQ ID NOs: 371, 364) and anti-VEGFR-TIM3-SIRPa (e.g., SEQ ID NOs: 382, 375).

In some embodiments, the fusion protein comprises TIM3 ECD and a polypeptide that inhibits a T cell co-inhibitory molecule. In specific embodiments, the fusion protein comprises TIM3 ECD and an antibody that binds and disables a T cell co-inhibitory molecule.

In some embodiments, the fusion protein comprises TIM3 ECD and a polypeptide that inhibits the interaction of TIGIT or PVRIG with PVRL2 or PVR. In some embodiments, this fusion protein comprises an antibody that binds TIGIT or PVRIG fused to TIM3 ECD. Exemplary embodiments of this fusion protein include anti-TIGIT-TIM3 (e.g., SEQ ID NOs: 479, 139) and anti-PVRIG-TIM3.

In some embodiments, the fusion protein comprises TIM3 ECD and a polypeptide that inhibits the interaction of VISTA and VSIG8. In some embodiments, this fusion protein comprises an antibody that binds VISTA or VSIG8 fused to TIM3 ECD. Exemplary embodiments of this fusion protein include anti-VISTA-TIM3 and anti-VSIG8-TIM3.

In some embodiments, the fusion protein comprises TIM3 ECD and a polypeptide that inhibits the interaction of PD-1 and PD-L1. In some embodiments, this fusion protein comprises an antibody that binds PD-1 or PD-L1 fused to TIM3 ECD. Exemplary embodiments of this fusion protein include anti-PD1-TIM3 (e.g., SEQ ID NOs: 457, 101) and anti-PDL1-TIM3 (e.g., SEQ ID NOs: 467, 109).

In some embodiments, the fusion protein comprises TIM3 ECD and a polypeptide that inhibits CTLA-4. In some embodiments, this fusion protein comprises an antibody that binds CTLA-4 fused to TIM3 ECD. Exemplary embodiments of this fusion protein include anti-CTLA4-TIM3 (e.g., SEQ ID NOs: 445, 28).

In some embodiments, the fusion protein comprises TIM3 ECD, an antibody that binds and disables a T cell co-inhibitory molecule, and an additional receptor ECD. In one aspect, the TIM3 ECD is fused to the heavy chain of the antibody and the additional receptor ECD is fused to the light chain of the antibody. Alternatively, the TIM3 ECD is fused to the light chain and the additional receptor ECD is fused to the heavy chain.

In some embodiments, the fusion protein comprises TIM3 ECD, an antibody that binds and disables a T cell co-inhibitory molecule, and PD1 ECD. Exemplary embodiments of this fusion protein include anti-CTLA4-TIM3-PD1 (e.g., SEQ ID NOs: 445, 436); anti-PD1-TIM3-PD1 (e.g., SEQ ID NOs: 457, 448); anti-TIGIT-TIM3-PD1 (e.g., SEQ ID NOs: 479, 470); anti-TIM3-TIM3-PD1 (e.g., SEQ ID NOs: 491, 482).

In other embodiments, the fusion protein comprises TIM3 ECD, an antibody that binds and disables a T cell co-inhibitory molecule, and SIRPa ECD. Exemplary embodiments of this fusion protein include anti-PD1-TIM3-SIRPa (e.g., SEQ ID NOs: 457, 450); anti-PDL1-TIM3-SIRPa (e.g., SEQ ID NOs: 467, 461); anti-TIGIT-TIM3-SIRPa (e.g., SEQ ID NOs: 479, 472); anti-CTLA4-TIM3-SIRPa (e.g., SEQ ID NOs: 445, 438); anti-TIM3-TIM3-SIRPa (e.g., SEQ ID NOs: 491, 484).

In other embodiments, the fusion protein comprises TIM3 ECD, an antibody that binds and disables a T cell co-inhibitory molecule, and SIGLEC10 ECD. Exemplary embodiments of this fusion protein include anti-TIGIT-TIM3-SIGLEC10 (e.g., SEQ ID NOs: 479, 471); anti-TIM3-TIM3-SIGLEC10 (e.g., SEQ ID NOs: 491, 483); anti-PDL1-TIM3-SIGLEC10 (e.g., SEQ ID NOs: 467, 460); anti-CTLA4-TIM3-SIGLEC10 (e.g., SEQ ID NOs: 445, 437); anti-PD1-TIM3-SIGLEC10 (e.g., SEQ ID NOs: 457, 449).

In other embodiments, the fusion protein comprises TIM3 ECD, an antibody that binds and disables a T cell co-inhibitory molecule, and VEGFR ECD. Exemplary embodiments of this fusion protein include anti-PDL1-VEGFR-TIM3 (e.g., SEQ ID NOs: 468, 462); anti-CTLA4-VEGFR-TIM3 (e.g., SEQ ID NOs: 446, 439); anti-TIM3-VEGFR-TIM3 (e.g., SEQ ID NOs: 492, 485); anti-PD1-VEGFR-TIM3 (e.g., SEQ ID NOs: 458, 451); anti-TIGIT-VEGFR-TIM3 (e.g., SEQ ID NOs: 480, 473)

In some embodiments, the fusion protein comprises an antibody that binds a T cell co-stimulatory molecule fused to TIM3. This antibody is preferably an agonist of the T cell co-stimulatory molecule. In some embodiments, this fusion protein is selected from: anti-41BB-TIM3 (e.g., SEQ ID NOs: 503, 2); anti-OX40-TIM3 (e.g., SEQ ID NOs: 515, 97); anti-ICOS-TIM3 (e.g., SEQ ID NOs: 527, 59).

In a further aspect, the fusion protein comprises an antibody that binds a T cell-costimulatory molecule, TIM3 ECD, and an additional receptor ECD. In one aspect, the TIM3 ECD is fused to the heavy chain of the antibody and the additional receptor ECD is fused to the light chain of the antibody. Alternatively, the TIM3 ECD is fused to the light chain of the antibody and the additional receptor ECD is fused to the heavy chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-ICOS-TIM3-PD1 (e.g., SEQ ID NOs: 527, 518); anti-41BB-TIM3-PD1 (e.g., SEQ ID NOs: 503, 494); anti-OX40-TIM3-PD1 (e.g., SEQ ID NOs: 515, 506). In another aspect, the additional receptor ECD is SIRPa ECD. In some embodiments, the fusion protein is selected from: anti-41BB-TIM3-SIRPa (e.g., SEQ ID NOs: 503, 496); anti-ICOS-TIM3-SIRPa (e.g., SEQ ID NOs: 527, 520); anti-OX40-TIM3-SIRPa (e.g., SEQ ID NOs: 515, 508). In another aspect, the additional receptor ECD is SIGLEC10 ECD. In some embodiments, the fusion protein is selected from: anti-ICOS-TIM3-SIGLEC10 (e.g., SEQ ID NOs: 527, 519); anti-41BB-TIM3-SIGLEC10 (e.g., SEQ ID NOs: 503, 495); anti-OX40-TIM3-SIGLEC10 (e.g., SEQ ID NOs: 515, 507).

In some embodiments, the fusion protein comprises an antibody, TIM3 ECD, and the ECD of a T cell co-stimulatory molecule. In one aspect, the TIM3 ECD is fused to heavy chain and the ECD of a T cell co-stimulatory molecule fused to light chain. Alternatively, the TIM3 ECD is fused to the light chain and the T cell co-stimulatory molecule ECD is fused to the heavy chain. In some embodiments, the fusion protein comprises TIM3 ECD and one of the following: OX40L, 41BBL, ICOSL.

In one embodiment, the fusion protein comprises TIM3 ECD and a polypeptide that binds an ectonucleotidase. In a preferred embodiment, the ectonucleotidase is either CD39 or CD73. In some embodiments, the fusion protein is an antibody that binds CD39 or CD73 fused to TIM3 ECD; for example: anti-CD39-TIM3 (e.g., SEQ ID NOs: 433, 18) or anti-CD73-TIM3 (e.g., SEQ ID NOs: 426, 24).

In some embodiments, the fusion protein comprises TIM3 ECD, an antibody that binds CD39 or CD73, and an additional receptor ECD. In some embodiments, the TIM3 ECD is fused to the heavy chain of the antibody and the additional receptor ECD is fused to the light chain of the antibody. Alternatively, the TIM3 ECD is fused to the light chain of the antibody and the additional receptor ECD is fused to the heavy chain of the antibody. In some embodiments, the fusion protein is selected from the following: anti-CD73-TIM3-SIRPa (e.g., SEQ ID NOs: 426, 419); anti-CD73-TIM3-PD1 (e.g., SEQ ID NOs: 426, 417).

In some embodiments, the fusion protein comprises a tumor-targeted antibody and TIM3 ECD. In some embodiments, this tumor targeted-antibody binds a tumor growth factor or growth factor receptor. In other embodiments, this tumor targeted antibody binds a tumor cell surface molecule. In some embodiments, this fusion protein is selected from the following: anti-EGFR-TIM3 (e.g., SEQ ID NOs: 230, 43), anti-HER2-TIM3 (e.g., SEQ ID NOs: 254, 55), anti-EGFRvIII-TIM3 (e.g., SEQ ID NOs: 242, 47), anti-uPAR-TIM3 (e.g., SEQ ID NOs: 273, 162), anti-PSMA-TIM3 (e.g., SEQ ID NOs: 280, 121), anti-nectin-4-TIM3.

In a further embodiment, the fusion protein comprises a tumor-targeted antibody, TIM3 ECD, and an additional receptor ECD. In one embodiment, the TIM3 ECD is fused to the heavy chain of the antibody and the additional receptor ECD is fused to the light chain of the antibody. Alternatively, the TIM3 ECD is fused to the light chain and the additional receptor ECD is fused to the heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-HER2-TIM3-PD1 (e.g., SEQ ID NOs: 254, 245); anti-EGFR-TIM3-PD1 (e.g., SEQ ID NOs: 230, 221); anti-nectin4-TIM3-PD1 (e.g., SEQ ID NOs: 266, 257); anti-EGFRvIII-TIM3-PD1 (e.g., SEQ ID NOs: 242, 233). In another aspect, the additional receptor ECD is SIRPa ECD. In some embodiments, the fusion protein is selected from: anti-EGFR-TIM3-SIRPa (e.g., SEQ ID NOs: 230, 223); anti-nectin4-TIM3-SIRPa (e.g., SEQ ID NOs: 266, 259); anti-HER2-TIM3-SIRPa (e.g., SEQ ID NOs: 254, 247); anti-EGFRvIII-TIM3-SIRPa (e.g., SEQ ID NOs: 242, 235). In another aspect, the additional receptor ECD is SIGLEC10 ECD. In some embodiments, the fusion protein is selected from: anti-HER2-TIM3-SIGLEC10 (e.g., SEQ ID NOs: 254, 246); anti-nectin4-TIM3-SIGLEC10 (e.g., SEQ ID NOs: 266, 258); anti-EGFR-TIM3-SIGLEC10 (e.g., SEQ ID NOs: 230, 222); anti-EGFRvIII-TIM3-SIGLEC10 (e.g., SEQ ID NOs: 242, 234). In another aspect, the additional receptor ECD is VEGFR ECD. In some embodiments, the fusion protein is selected from: anti-EGFR-VEGFR-TIM3 (e.g., SEQ ID NOs: 231, 224); anti-EGFRvIII-VEGFR-TIM3 (e.g., SEQ ID NOs: 243, 236); anti-HER2-VEGFR-TIM3 (e.g., SEQ ID NOs: 255, 248); anti-nectin4-VEGFR-TIM3 (e.g., SEQ ID NOs: 267, 260).

In some embodiments, the fusion protein comprises TIM3 ECD and a polypeptide that inhibits VEGF/VEGFR signaling.

In some embodiments, the fusion protein comprises TIM3 ECD and anti-VEGFR mAb with TIM3 ECD fused to either the heavy chain or light chain of the antibody. In some embodiments, this fusion protein is anti-VEGFR-TIM3 (e.g., SEQ ID NOs: 382, 148). In a further embodiment, the fusion protein may additionally comprise a receptor ECD fused to the other chain of the antibody. In some embodiments, the fusion protein is selected from anti-VEGFR-TIM3-BTLA (e.g., SEQ ID NOs: 382, 378); anti-VEGFR-TIM3-SIRPa (e.g., SEQ ID NOs: 382, 375); anti-VEGFR-TIM3-SIGLEC10 (e.g., SEQ ID NOs: 382, 374); anti-VEGFR-TIM3-PD1 (e.g., SEQ ID NOs: 382, 373).

In some embodiments, the fusion protein comprises TIM3 ECD and anti-VEGF mAb with TIM3 ECD fused to either the heavy chain or light chain of the antibody. In some embodiments, this fusion protein is anti-VEGF-TIM3 (e.g., SEQ ID NOs: 371, 32). In a further embodiment, the fusion protein may additionally comprise a receptor ECD fused to the other chain of the antibody. In some embodiments, the fusion protein is selected from anti-VEGF-TIM3-SIGLEC10 (e.g., SEQ ID NOs: 371, 363); anti-VEGF-TIM3-BTLA (e.g., SEQ ID NOs: 371, 367); anti-VEGF-TIM3-PD1 (e.g., SEQ ID NOs: 371, 362); anti-VEGF-TIM3-SIRPa (e.g., SEQ ID NOs: 371, 364).

In other embodiments, the fusion protein comprises TIM3 ECD and VEGFR ECD. In one embodiment, this fusion protein is TIM3-Fc-VEGFR (e.g., SEQ ID NO: 564). In another embodiment, this fusion protein is VEGFR-Fc-TIM3 (e.g., SEQ ID NO: 570).

In some embodiments, the fusion protein comprises an antibody that binds IL-17 or IL-17R and TIM3 ECD. In some embodiments, this fusion protein is selected from the following: anti-IL17-TIM3, anti-IL17R-TIM3 (e.g., SEQ ID NOs: 335, 63).

In a further embodiment, the fusion protein comprises an antibody that binds IL-17 or IL-17R, TIM3 ECD, and an additional receptor ECD. In one embodiment, TIM3 ECD is fused to the heavy chain of the antibody and the additional receptor ECD is fused to the light chain of the antibody. Alternatively, TIM3 ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL17R-TIM3-PD1 (e.g., SEQ ID NOs: 335, 326). In another aspect, the additional receptor ECD is SIRPa ECD. In some embodiments, the fusion protein is selected from: anti-IL17R-TIM3-SIRPa (e.g., SEQ ID NOs: 335, 328). In another aspect, the additional receptor ECD is SIGLEC10 ECD. In some embodiments, the fusion protein is selected from: anti-IL17R-TIM3-SIGLEC10 (e.g., SEQ ID NOs: 335, 327). In another aspect, the additional receptor ECD is VEGFR ECD. In some embodiments, the fusion protein is selected from: anti-IL6R-VEGFR-TIM3 (e.g., SEQ ID NOs: 324, 317).

In some embodiments, the fusion protein comprises an antibody that binds IL-23 or IL-23R and TIM3 ECD. If the antibody binds IL-23, it is preferred that the antibody bind the p19 subunit of IL-23 that is not shared with IL-12. In some embodiments, this fusion protein is selected from the following: anti-IL23-TIM3 (e.g., SEQ ID NOs: 347, 75), anti-IL23R-TIM3.

In a further embodiment, the fusion protein comprises an antibody that binds IL-23 or IL-23R, TIM3 ECD, and an additional receptor ECD. In one embodiment, TIM3 ECD is fused to the heavy chain of the antibody and the additional receptor ECD is fused to the light chain of the antibody. Alternatively, TIM3 ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL23-TIM3-PD1 (e.g., SEQ ID NOs: 347, 338). In another aspect, the additional receptor ECD is SIRPa ECD. In some embodiments, the fusion protein is selected from: anti-IL23-TIM3-SIRPa (e.g., SEQ ID NOs: 347, 340). In another aspect, the additional receptor ECD is SIGLEC10 ECD. In some embodiments, the fusion protein is selected from: anti-IL23-TIM3-SIGLEC10 (e.g., SEQ ID NOs: 347, 339). In another aspect, the additional receptor ECD is VEGFR ECD. In some embodiments, the fusion protein is selected from: anti-IL23-VEGFR-TIM3 (e.g., SEQ ID NOs: 348, 341).

In some embodiments, the fusion protein comprises an antibody that binds IL-6 or IL-6R and TIM3 ECD. In some embodiments, this fusion protein is selected from the following: anti-IL6-TIM3, anti-IL6R-TIM3 (e.g., SEQ ID NOs: 323, 79).

In some embodiments, the fusion protein comprises an antibody that binds IL-6 or IL-6R, TIM3 ECD, and an additional receptor ECD. In one embodiment, TIM3 ECD is fused to the heavy chain of the antibody and the additional receptor ECD is fused to the light chain of the antibody. Alternatively, TIM3 ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL6R-TIM3-PD1 (e.g., SEQ ID NOs: 323, 314). In another aspect, the additional receptor ECD is SIRPa ECD. In some embodiments, the fusion protein is selected from: anti-IL6R-TIM3-SIRPa (e.g., SEQ ID NOs: 323, 316). In another aspect, the additional receptor ECD is SIGLEC10 ECD. In some embodiments, the fusion protein is selected from: anti-IL6R-TIM3-SIGLEC10 (e.g., SEQ ID NOs: 323, 315). In another aspect, the additional receptor ECD is VEGFR ECD. In some embodiments, the fusion protein is selected from: anti-IL6R-VEGFR-TIM3 (e.g., SEQ ID NOs: 324, 317).

In some embodiments, the fusion protein comprises TIM3 ECD and an antibody that binds TGFb, TGFbR, or GARP. Exemplary embodiments of this fusion protein include anti-TGFb-TIM3 (e.g., SEQ ID NOs: 402, 133), anti-TGFbR-TIM3, and anti-GARP-TIM3 (e.g., SEQ ID NOs: 414, 49).

In some embodiments, the fusion protein further comprises an additional receptor ECD. Exemplary embodiments of this fusion protein include anti-TGFb-TIM3-SIGLEC10 (e.g., SEQ ID NOs: 402, 395); anti-TGFb-TIM3-BTLA (e.g., SEQ ID NOs: 402, 399); anti-TGFb-TIM3-PD1 (e.g., SEQ ID NOs: 402, 394); anti-TGFb-TIM3-SIRPa (e.g., SEQ ID NOs: 402, 396).

In some embodiments, the fusion protein comprises TIM3 ECD and IL-15. In some embodiments, the fusion protein is IL15-Fc-TIM3 (e.g., SEQ ID NO: 594) or TIM3-Fc-IL15 (e.g., SEQ ID NO: 593). In other embodiments, the fusion protein is IL12-Fc-TIM3 (e.g., SEQ ID NO: 592) or TIM3-Fc-IL12 (e.g., SEQ ID NO: 591). In other embodiments, the fusion protein comprises an antibody with TIM3 ECD fused to heavy chain and IL-15 fused to light chain. In other embodiments, the fusion protein comprises an antibody with TIM3 ECD fused to heavy chain and IL-12 fused to light chain.

In various embodiments, the fusion proteins of the invention counteract BTLA/HVEM in the tumor microenvironment. In some embodiments, the fusion proteins comprise a ligand-binding sequence of an extracellular domain of BTLA (e.g., BTLA ECD).

In one embodiment, the fusion protein of the invention is an ALT comprising an antibody that binds BTLA or BTLA ligand (e.g. HVEM). In one example, the ALT comprises an antibody that binds BTLA and interferes with its interaction with HVEM. In one example, the ALT comprises an antibody that binds HVEM and interferes with its interaction with BTLA. In one aspect, the antibody is an antagonist that inhibits BTLA intracellular ITIM or ITSM signaling, thereby promoting immune cell activation. In various examples, the antibody that binds BTLA or HVEM is fused to one or more ligand traps. In one example the BTLA or HVEM antibody is fused to a ligand-binding sequence of the extracellular domain of PD1 (PD1 ECD). In one example the BTLA or HVEM antibody is fused to a ligand-binding sequence of the extracellular domain of TGFbR (TGFbRII ECD). In one example the BTLA or HVEM antibody is fused to a ligand-binding sequence of the extracellular domain of TIM3 (TIM3 ECD). In one example the BTLA or HVEM antibody is fused to a ligand-binding sequence of the extracellular domain of SIRPa (SIRPa ECD). In one example the BTLA or HVEM antibody is fused to a ligand-binding sequence of the extracellular domain of VEGFR (VEGFR ECD). In one embodiment, the BTLA or HVEM binding antibody is fused to multiple ligand traps selected from the following: PD1 ECD, TGFbRII ECD, TIM3 ECD, SIRPa ECD, VEGFR ECD. In one aspect, a ligand trap (LT1) is fused to the heavy chain, and a second ligand trap (LT2) is fused to the light chain.

In various examples, the ALT comprises a BTLA or HVEM binding antibody, wherein the heavy chain is fused to TGFbRII ECD and the light chain is fused to PD1 ECD, TIM3 ECD, SIRPa ECD, or SIGLEC10 ECD. In various examples, the ALT comprises a BTLA or HVEM binding antibody, wherein the heavy chain is fused to VEGFR ECD and the light chain is fused to PD1 ECD, TIM3 ECD, SIRPa ECD, or SIGLEC10 ECD. In various examples, the ALT comprises a BTLA or HVEM binding antibody, wherein the heavy chain is fused to PD1 ECD and the light chain is fused to TIM3 ECD, SIRPa ECD, or SIGLEC10 ECD. In various examples, the ALT comprises a BTLA or HVEM binding antibody, wherein the heavy chain is fused to TIM3 ECD and the light chain is fused to PD1 ECD, SIRPa ECD, or SIGLEC10 ECD. In various examples, the ALT comprises a BTLA or HVEM binding antibody, wherein the heavy chain is fused to SIRPa ECD and the light chain is fused to TIM3 ECD, PD1 ECD, or SIGLEC10 ECD. In various examples, the ALT comprises a BTLA or HVEM binding antibody, wherein the heavy chain is fused to SIGLEC10 ECD and the light chain is fused to TIM3 ECD, SIRPa ECD, or PD1 ECD. In another embodiment, the molecule contains a ligand-binding sequence of the extracellular domain of TIM3 (TIM3 ECD). In one aspect, the TIM3 ECD binds and disables TIM3 ligands (e.g. CEACAM1). In one aspect, the TIM3 ECD interferes with the interaction of TIM3 with CEACAM1 or homodimerization of CEACAM1.

In one embodiment, the fusion protein comprises a ligand-binding sequence of an extracellular domain of B- and T-lymphocyte attenuator (BTLA ECD). In some embodiments, the fusion protein comprises an antibody and BTLA ECD. In one embodiment, the BTLA ECD is fused to the heavy chain of the antibody. In another embodiment, the BTLA ECD is fused to the light chain of the antibody.

In some embodiments, the fusion protein of the invention comprises a targeting polypeptide that is an antibody. In one embodiment, the antibody is fused to BTLA ECD and additional ligand traps selected from the following: PD1 ECD, TGFbRII ECD, TIM3 ECD, SIRPa ECD, VEGFR ECD, SIGLEC10 ECD. In one aspect, a ligand trap (LT1) is fused to the heavy chain, and a second ligand trap (LT2) is fused to the light chain. In one aspect, the BTLA ECD is fused to the heavy chain and another ligand trap is fused to the light chain. In one aspect, the BTLA ECD is fused to the light chain and another ligand trap is fused to the heavy chain. In various examples, the ALT comprises an antibody, wherein the heavy chain is fused to TIM3 ECD and the light chain is fused to one of PD1 ECD, TIM3 ECD, SIRPa ECD, or SIGLEC10 ECD. In various examples, the ALT comprises an antibody wherein the light chain is fused to TIM3 ECD and the heavy chain is fused to one of PD1 ECD, TGFbRII ECD, TIM3 ECD, SIRPa ECD, VEGFR ECD, SIGLEC10 ECD.

In some embodiments, the fusion protein comprises BTLA ECD and a polypeptide that inhibits the interaction between CD47 and SIRPa. In one embodiment, this fusion protein comprises BTLA ECD and a polypeptide that binds CD47. In one embodiment, this fusion protein is anti-CD47 mAb fused to BTLA ECD (anti-CD47-BTLA (e.g., SEQ ID NOs: 383, 22)). In another embodiment, this fusion protein comprises SIRPa ECD and BTLA ECD. In one embodiment, this fusion protein is SIRPa-Fc-BTLA (e.g., SEQ ID NO: 547) or BTLA-Fc-SIRPa (e.g., SEQ ID NO: 531).

In a further aspect, the fusion protein comprises BTLA ECD, a polypeptide that inhibits the interaction between CD47 and SIRPa, and another polypeptide that inhibits a T cell co-inhibitory molecule. In some embodiments, this fusion protein comprises anti-CD47 mAb with BTLA ECD fused to the heavy chain or light chain; and a T cell co-inhibitory molecule ECD fused to the other chain. In a particular embodiment, this fusion protein is anti-CD47-BTLA-PD1 (e.g., SEQ ID NOs: 383, 384). In another embodiment, this fusion protein is anti-CD47-BTLA-TIM3 (e.g., SEQ ID NOs: 383, 386).

In another aspect, the fusion protein comprises BTLA ECD, anti-CD47 mAb, and VEGFR ECD. In some embodiments, VEGFR ECD is fused to heavy chain of anti-CD47 mAb and BTLA ECD is fused to light chain of anti-CD47 mAb. In one embodiment, this fusion protein is anti-CD47-VEGFR-BTLA (e.g., SEQ ID NOs: 392, 388).

In another aspect, the fusion protein comprises BTLA ECD, SIRPa ECD, and an antibody with a heavy chain and light chain. In one aspect, the BTLA ECD is fused to the heavy chain of the antibody and the SIRPa ECD is fused to the light chain of the antibody. In another aspect, the BTLA ECD is fused to the light chain of the antibody and the SIRPa ECD is fused to the heavy chain of the antibody. In some embodiments, the antibody of said fusion protein binds a T cell co-inhibitory molecule as an antagonist. Exemplary embodiments of such fusion proteins include anti-CTLA4-BTLA-SIRPa (e.g., SEQ ID NOs: 435, 438), anti-PD1-BTLA-SIRPa (e.g., SEQ ID NOs: 447, 450), and anti-PDL1-BTLA-SIRPa (e.g., SEQ ID NOs: 459, 461). In other embodiments, the antibody of said fusion protein binds a T cell co-stimulatory molecule as an agonist. Exemplary embodiments of this fusion protein include anti-OX40-BTLA-SIRPa (e.g., SEQ ID NOs: 505, 508), anti-41BB-BTLA-SIRPa (e.g., SEQ ID NOs: 493, 496), and anti-CD40-BTLA-SIRPa. In other embodiments, the antibody of said fusion protein is a tumor-targeted antibody. In some embodiments, this tumor targeted-antibody binds a tumor growth factor or growth factor receptor. In other embodiments, this tumor targeted antibody binds a tumor cell surface molecule. Exemplary embodiments of this fusion protein include anti-EGFR-BTLA-SIRPa (e.g., SEQ ID NOs: 220, 223), anti-HER2-BTLA-SIRPa (e.g., SEQ ID NOs: 244, 247), anti-EGFRvIII-BTLA-SIRPa (e.g., SEQ ID NOs: 232, 235), anti-uPAR-BTLA-SIRPa, and anti-PSMA-BTLA-SIRPa.

In other embodiments, the antibody of said fusion protein binds a member of the TGFb pathway. In some embodiments, this antibody binds TGFb, TGFbR, or GARP. Exemplary embodiments of this fusion protein include anti-TGFb-BTLA-SIRPa (e.g., SEQ ID NOs: 393, 396), anti-TGFbR-BTLA-SIRPa, and anti-GARP-BTLA-SIRPa.

In other embodiments, the antibody of said fusion protein binds VEGF or VEGFR. Exemplary embodiments of this fusion protein include anti-VEGF-BTLA-SIRPa (e.g., SEQ ID NOs: 361, 364) and anti-VEGFR-BTLA-SIRPa (e.g., SEQ ID NOs: 372, 375).

In some embodiments, the fusion protein comprises BTLA ECD and a polypeptide that inhibits a T cell co-inhibitory molecule. In specific embodiments, the fusion protein comprises BTLA ECD and an antibody that binds and disables a T cell co-inhibitory molecule.

In some embodiments, the fusion protein comprises BTLA ECD and a polypeptide that inhibits the interaction of TIGIT or PVRIG with PVRL2 or PVR. In some embodiments, this fusion protein comprises an antibody that binds TIGIT or PVRIG fused to BTLA ECD. Exemplary embodiments of this fusion protein include anti-TIGIT-BTLA (e.g., SEQ ID NOs: 469, 139) and anti-PVRIG-BTLA.

In some embodiments, the fusion protein comprises BTLA ECD and a polypeptide that inhibits the interaction of VISTA and VSIG8. In some embodiments, this fusion protein comprises an antibody that binds VISTA or VSIG8 fused to BTLA ECD. Exemplary embodiments of this fusion protein include anti-VISTA-BTLA and anti-VSIG8-BTLA.

In some embodiments, the fusion protein comprises BTLA ECD and a polypeptide that inhibits the interaction of PD-1 and PD-L1. In some embodiments, this fusion protein comprises an antibody that binds PD-1 or PD-L1 fused to BTLA ECD. Exemplary embodiments of this fusion protein include anti-PD1-BTLA (e.g., SEQ ID NOs: 447, 101) and anti-PDL1-BTLA (e.g., SEQ ID NOs: 459, 109) and anti-PDL1-TGFbRII-BTLA (e.g., SEQ ID NOs: 466, 464). In other embodiments, the polypeptide that inhibits the interaction of PD-1/PD-L1 is PD1 ECD. In one aspect, the fusion protein comprises Fc, BTLA ECD, and PD-1 ECD; and has the structure N-BTLA ECD-Fc-PD1 ECD-C, or N-PD1 ECD-Fc-BTLA ECD.

In some embodiments, the fusion protein comprises BTLA ECD and a polypeptide that inhibits CTLA-4. In some embodiments, this fusion protein comprises an antibody that binds CTLA-4 fused to BTLA ECD. Exemplary embodiments of this fusion protein include anti-CTLA4-BTLA (e.g., SEQ ID NOs: 435, 28).

In some embodiments, the fusion protein comprises BTLA ECD and a polypeptide that inhibits TIM-3. In some embodiments, this fusion protein comprises an antibody that binds TIM-3 fused to BTLA ECD. Exemplary embodiments of this fusion protein include anti-TIM3-BTLA (e.g., SEQ ID NOs: 481, 141).

In some embodiments, the fusion protein comprises BTLA ECD and a polypeptide that binds CEACAM1 and/or CEACAM5. In some embodiments, this fusion protein comprises an antibody that binds CEACAM1 or CEACAM5 fused to BTLA ECD. Exemplary embodiments of this fusion protein include anti-CEACAM5-BTLA (e.g., SEQ ID NOs: 282, 26). In other embodiments, this CEACAM-binding polypeptide is TIM3 ECD.

Fusion proteins comprising BTLA ECD and mAb that inhibits a T cell co-inhibitory molecule and additional receptor ECD

In some embodiments, the fusion protein comprises BTLA ECD, an antibody that binds and disables a T cell co-inhibitory molecule, and an additional receptor ECD. In one aspect, the BTLA ECD is fused to the heavy chain of the antibody and the additional receptor ECD is fused to the light chain of the antibody. Alternatively, the BTLA ECD is fused to the light chain and the additional receptor ECD is fused to the heavy chain.

In some embodiments, the fusion protein comprises BTLA ECD, an antibody that binds and disables a T cell co-inhibitory molecule, and PD1 ECD. In some embodiments, BTLA is fused to the light chain and PD-1 is fused to the heavy chain. In other embodiments, BTLA is fused to the heavy chain and PD-1 is fused to the light chain. Exemplary embodiments of this fusion protein include anti-PD1-BTLA-PD1 (e.g., SEQ ID NOs: 447, 448); anti-CTLA4-BTLA-PD1 (e.g., SEQ ID NOs: 435, 436); anti-TIGIT-BTLA-PD1 (e.g., SEQ ID NOs: 469, 470); anti-TIM3-BTLA-PD1 (e.g., SEQ ID NOs: 481, 482).

In other embodiments, the fusion protein comprises BTLA ECD, an antibody that binds and disables a T cell co-inhibitory molecule, and TIM3 ECD. Exemplary embodiments of this fusion protein include anti-TIM3-BTLA-TIM3 (e.g., SEQ ID NOs: 481, 485); anti-CTLA4-BTLA-TIM3 (e.g., SEQ ID NOs: 435, 439); anti-TIGIT-BTLA-TIM3 (e.g., SEQ ID NOs: 469, 473); anti-PDL1-BTLA-TIM3 (e.g., SEQ ID NOs: 459, 462); anti-PD1-BTLA-TIM3 (e.g., SEQ ID NOs: 447, 451).

In other embodiments, the fusion protein comprises BTLA ECD, an antibody that binds and disables a T cell co-inhibitory molecule, and SIRPa ECD. Exemplary embodiments of this fusion protein include anti-TIGIT-BTLA-SIRPa (e.g., SEQ ID NOs: 469, 472); anti-PDL1-BTLA-SIRPa (e.g., SEQ ID NOs: 459, 461); anti-CTLA4-BTLA-SIRPa (e.g., SEQ ID NOs: 435, 438); anti-TIM3-BTLA-SIRPa (e.g., SEQ ID NOs: 481, 484); anti-PD1-BTLA-SIRPa (e.g., SEQ ID NOs: 447, 450).

In other embodiments, the fusion protein comprises BTLA ECD, an antibody that binds and disables a T cell co-inhibitory molecule, and SIGLEC10 ECD. Exemplary embodiments of this fusion protein include anti-CTLA4-BTLA-SIGLEC10 (e.g., SEQ ID NOs: 435, 437); anti-TIM3-BTLA-SIGLEC10 (e.g., SEQ ID NOs: 481, 483); anti-TIGIT-BTLA-SIGLEC10 (e.g., SEQ ID NOs: 469, 471); anti-PD1-BTLA-SIGLEC10 (e.g., SEQ ID NOs: 447, 449); anti-PDL1-BTLA-SIGLEC10 (e.g., SEQ ID NOs: 459, 460).

In other embodiments, the fusion protein comprises BTLA ECD, an antibody that binds and disables a T cell co-inhibitory molecule, and VEGFR ECD. Exemplary embodiments of this fusion protein include anti-TIGIT-VEGFR-BTLA (e.g., SEQ ID NOs: 480, 475); anti-TIM3-VEGFR-BTLA (e.g., SEQ ID NOs: 492, 487); anti-PDL1-VEGFR-BTLA (e.g., SEQ ID NOs: 468, 464); anti-PD1-VEGFR-BTLA (e.g., SEQ ID NOs: 458, 453); anti-CTLA4-VEGFR-BTLA (e.g., SEQ ID NOs: 446, 441).

In some embodiments, the fusion protein comprises an antibody that binds a T cell co-stimulatory molecule fused to BTLA. This antibody is preferably an agonist of the T cell co-stimulatory molecule. In some embodiments, this fusion protein is selected from: anti-41BB-BTLA (e.g., SEQ ID NOs: 493, 2); anti-ICOS-BTLA (e.g., SEQ ID NOs: 517, 59); anti-OX40-BTLA (e.g., SEQ ID NOs: 505, 97).

In a further aspect, the fusion protein comprises an antibody that binds a T cell-costimulatory molecule, BTLA ECD, and an additional receptor ECD. In one aspect, the BTLA ECD is fused to the heavy chain of the antibody and the additional receptor ECD is fused to the light chain of the antibody. Alternatively, the BTLA ECD is fused to the light chain of the antibody and the additional receptor ECD is fused to the heavy chain of the antibody. In some embodiments, the additional receptor ECD is the ECD of a T cell co-inhibitory molecule. In one embodiment, the T cell co-inhibitory molecule is PD-1. In some embodiments, the fusion protein is selected from: anti-OX40-BTLA-PD1 (e.g., SEQ ID NOs: 505, 506); anti-ICOS-BTLA-PD1 (e.g., SEQ ID NOs: 517, 518); anti-41BB-BTLA-PD1 (e.g., SEQ ID NOs: 493, 494). In another embodiment, the T cell co-inhibitory molecule is TIM3. In some embodiments, the fusion protein is selected from: anti-ICOS-BTLA-TIM3 (e.g., SEQ ID NOs: 517, 521); anti-41BB-BTLA-TIM3 (e.g., SEQ ID NOs: 493, 497); anti-OX40-BTLA-TIM3 (e.g., SEQ ID NOs: 505, 509). In another aspect, the additional receptor ECD is SIRPa ECD. In some embodiments, the fusion protein is selected from: anti-ICOS-BTLA-SIRPa (e.g., SEQ ID NOs: 517, 520); anti-OX40-BTLA-SIRPa (e.g., SEQ ID NOs: 505, 508); anti-41BB-BTLA-SIRPa (e.g., SEQ ID NOs: 493, 496). In another aspect, the additional receptor ECD is SIGLEC10 ECD. In some embodiments, the fusion protein is selected from: anti-41BB-BTLA-SIGLEC10 (e.g., SEQ ID NOs: 493, 495); anti-ICOS-BTLA-SIGLEC10 (e.g., SEQ ID NOs: 517, 519); anti-OX40-BTLA-SIGLEC10 (e.g., SEQ ID NOs: 505, 507).

Fusion proteins comprising BTLA ECD and mAb and T cell co-stimulatory ECD

In some embodiments, the fusion protein comprises an antibody, BTLA ECD, and the ECD of a T cell co-stimulatory molecule. In one aspect, the BTLA ECD is fused to heavy chain and the ECD of a T cell co-stimulatory molecule fused to light chain. Alternatively, the BTLA ECD is fused to the light chain and the T cell co-stimulatory molecule ECD is fused to the heavy chain. In some embodiments, the fusion protein comprises BTLA ECD and one of the following: OX40L, 41BBL, ICOSL.

In one embodiment, the fusion protein comprises BTLA ECD and a polypeptide that binds an ectonucleotidase. In a preferred embodiment, the ectonucleotidase is either CD39 or CD73. In some embodiments, the fusion protein is an antibody that binds CD39 or CD73 fused to BTLA ECD; for example: anti-CD39-BTLA (e.g., SEQ ID NOs: 428, 18) or anti-CD73-BTLA (e.g., SEQ ID NOs: 416, 24).

In some embodiments, the fusion protein comprises BTLA ECD, an antibody that binds CD39 or CD73, and an additional receptor ECD. In some embodiments, the BTLA ECD is fused to the heavy chain of the antibody and the additional receptor ECD is fused to the light chain of the antibody. Alternatively, the BTLA ECD is fused to the light chain of the antibody and the additional receptor ECD is fused to the heavy chain of the antibody. In some embodiments, the fusion protein is selected from the following: anti-CD73-BTLA-SIRPa (e.g., SEQ ID NOs: 416, 419); anti-CD73-BTLA-TIM3 (e.g., SEQ ID NOs: 416, 420); anti-CD73-BTLA-PD1 (e.g., SEQ ID NOs: 416, 417).

In some embodiments, the fusion protein comprises a tumor-targeted antibody and BTLA ECD. In some embodiments, this tumor targeted-antibody binds a tumor growth factor or growth factor receptor. In other embodiments, this tumor targeted antibody binds a tumor cell surface molecule. In some embodiments, this fusion protein is selected from the following: anti-EGFR-BTLA (e.g., SEQ ID NOs: 220, 43), anti-HER2-BTLA (e.g., SEQ ID NOs: 244, 55), anti-EGFRvIII-BTLA (e.g., SEQ ID NOs: 232, 47), anti-uPAR-BTLA (e.g., SEQ ID NOs: 268, 162), anti-PSMA-BTLA (e.g., SEQ ID NOs: 275, 121), anti-nectin-4-BTLA.

In a further embodiment, the fusion protein comprises a tumor-targeted antibody, BTLA ECD, and an additional receptor ECD. In one embodiment, the BTLA ECD is fused to the heavy chain of the antibody and the additional receptor ECD is fused to the light chain of the antibody. Alternatively, the BTLA ECD is fused to the light chain and the additional receptor ECD is fused to the heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-HER2-BTLA-PD1 (e.g., SEQ ID NOs: 244, 245); anti-EGFR-BTLA-PD1 (e.g., SEQ ID NOs: 220, 221); anti-EGFRvIII-BTLA-PD1 (e.g., SEQ ID NOs: 232, 233); anti-nectin4-BTLA-PD1 (e.g., SEQ ID NOs: 256, 257). In another embodiment, the T cell co-inhibitory molecule is TIM3. In some embodiments, the fusion protein is selected from: anti-EGFRvIII-BTLA-TIM3 (e.g., SEQ ID NOs: 232, 236); anti-nectin4-BTLA-TIM3 (e.g., SEQ ID NOs: 256, 260); anti-EGFR-BTLA-TIM3 (e.g., SEQ ID NOs: 220, 224); anti-HER2-BTLA-TIM3 (e.g., SEQ ID NOs: 244, 248). In another aspect, the additional receptor ECD is SIRPa ECD. In some embodiments, the fusion protein is selected from: anti-EGFRvIII-BTLA-SIRPa (e.g., SEQ ID NOs: 232, 235); anti-nectin4-BTLA-SIRPa (e.g., SEQ ID NOs: 256, 259); anti-HER2-BTLA-SIRPa (e.g., SEQ ID NOs: 244, 247); anti-EGFR-BTLA-SIRPa (e.g., SEQ ID NOs: 220, 223). In another aspect, the additional receptor ECD is SIGLEC10 ECD. In some embodiments, the fusion protein is selected from: anti-EGFR-BTLA-SIGLEC10 (e.g., SEQ ID NOs: 220, 222); anti-nectin4-BTLA-SIGLEC10 (e.g., SEQ ID NOs: 256, 258); anti-HER2-BTLA-SIGLEC10 (e.g., SEQ ID NOs: 244, 246); anti-EGFRvIII-BTLA-SIGLEC10 (e.g., SEQ ID NOs: 232, 234). In another aspect, the additional receptor ECD is VEGFR ECD. In some embodiments, the fusion protein is selected from: anti-nectin4-VEGFR-BTLA (e.g., SEQ ID NOs: 267, 262); anti-EGFR-VEGFR-BTLA (e.g., SEQ ID NOs: 231, 226); anti-HER2-VEGFR-BTLA (e.g., SEQ ID NOs: 255, 250); anti-EGFRvIII-VEGFR-BTLA (e.g., SEQ ID NOs: 243, 238).

In some embodiments, the fusion protein comprises BTLA ECD and a polypeptide that inhibits VEGF/VEGFR signaling.

In some embodiments, the fusion protein comprises BTLA ECD and anti-VEGFR mAb with BTLA ECD fused to either the heavy chain or light chain of the antibody. In some embodiments, this fusion protein is anti-VEGFR-BTLA (e.g., SEQ ID NOs: 372, 148). In a further embodiment, the fusion protein may additionally comprise a receptor ECD fused to the other chain of the antibody. In some embodiments, the fusion protein is selected from anti-VEGFR-BTLA-SIRPa (e.g., SEQ ID NOs: 372, 375); anti-VEGFR-BTLA-PD1 (e.g., SEQ ID NOs: 372, 373); anti-VEGFR-BTLA-TIM3 (e.g., SEQ ID NOs: 372, 376); anti-VEGFR-BTLA-SIGLEC10 (e.g., SEQ ID NOs: 372, 374).

In some embodiments, the fusion protein comprises BTLA ECD and anti-VEGF mAb with BTLA ECD fused to either the heavy chain or light chain of the antibody. In some embodiments, this fusion protein is anti-VEGF-BTLA (e.g., SEQ ID NOs: 361, 32). In a further embodiment, the fusion protein may additionally comprise a receptor ECD fused to the other chain of the antibody. In some embodiments, the fusion protein is selected from anti-VEGF-BTLA-SIGLEC10 (e.g., SEQ ID NOs: 361, 363); anti-VEGF-BTLA-SIRPa (e.g., SEQ ID NOs: 361, 364); anti-VEGF-BTLA-PD1 (e.g., SEQ ID NOs: 361, 362); anti-VEGF-BTLA-TIM3 (e.g., SEQ ID NOs: 361, 365).

In other embodiments, the fusion protein comprises BTLA ECD and VEGFR ECD. In one embodiment, this fusion protein is BTLA-Fc-VEGFR (e.g., SEQ ID NO: 534). In another embodiment, this fusion protein is VEGFR-Fc-BTLA (e.g., SEQ ID NO: 565).

In some embodiments, the fusion protein comprises an antibody that binds IL-17 or IL-17R and BTLA ECD. In some embodiments, this fusion protein is selected from the following: anti-IL17-BTLA, anti-IL17R-BTLA (e.g., SEQ ID NOs: 325, 63).

In a further embodiment, the fusion protein comprises an antibody that binds IL-17 or IL-17R, BTLA ECD, and an additional receptor ECD. In one embodiment, BTLA ECD is fused to the heavy chain of the antibody and the additional receptor ECD is fused to the light chain of the antibody. Alternatively, BTLA ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL17R-BTLA-PD1 (e.g., SEQ ID NOs: 325, 326). In another embodiment, the T cell co-inhibitory molecule is TIM3. In some embodiments, the fusion protein is selected from: anti-IL17R-BTLA-TIM3 (e.g., SEQ ID NOs: 325, 329). In another aspect, the additional receptor ECD is SIRPa ECD. In some embodiments, the fusion protein is selected from: anti-IL17R-BTLA-SIRPa (e.g., SEQ ID NOs: 325, 328). In another aspect, the additional receptor ECD is SIGLEC10 ECD. In some embodiments, the fusion protein is selected from: anti-IL17R-BTLA-SIGLEC10 (e.g., SEQ ID NOs: 325, 327). In another aspect, the additional receptor ECD is VEGFR ECD. In some embodiments, the fusion protein is selected from: anti-IL6R-VEGFR-BTLA (e.g., SEQ ID NOs: 324, 319).

In some embodiments, the fusion protein comprises an antibody that binds IL-23 or IL-23R and BTLA ECD. If the antibody binds IL-23, it is preferred that the antibody bind the p19 subunit of IL-23 that is not shared with IL-12. In some embodiments, this fusion protein is selected from the following: anti-IL23-BTLA (e.g., SEQ ID NOs: 337, 75), anti-IL23R-BTLA.

In a further embodiment, the fusion protein comprises an antibody that binds IL-23 or IL-23R, BTLA ECD, and an additional receptor ECD. In one embodiment, BTLA ECD is fused to the heavy chain of the antibody and the additional receptor ECD is fused to the light chain of the antibody. Alternatively, BTLA ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL23-BTLA-PD1 (e.g., SEQ ID NOs: 337, 338). In another embodiment, the T cell co-inhibitory molecule is TIM3. In some embodiments, the fusion protein is selected from: anti-IL23-BTLA-TIM3 (e.g., SEQ ID NOs: 337, 341). In another aspect, the additional receptor ECD is SIRPa ECD. In some embodiments, the fusion protein is selected from: anti-IL23-BTLA-SIRPa (e.g., SEQ ID NOs: 337, 340). In another aspect, the additional receptor ECD is SIGLEC10 ECD. In some embodiments, the fusion protein is selected from: anti-IL23-BTLA-SIGLEC10 (e.g., SEQ ID NOs: 337, 339). In another aspect, the additional receptor ECD is VEGFR ECD. In some embodiments, the fusion protein is selected from: anti-IL23-VEGFR-BTLA (e.g., SEQ ID NOs: 348, 343).

In some embodiments, the fusion protein comprises an antibody that binds IL-6 or IL-6R and BTLA ECD. In some embodiments, this fusion protein is selected from the following: anti-IL6-BTLA, anti-IL6R-BTLA (e.g., SEQ ID NOs: 313, 79).

In a further embodiment, the fusion protein comprises an antibody that binds IL-6 or IL-6R, BTLA ECD, and an additional receptor ECD. In one embodiment, BTLA ECD is fused to the heavy chain of the antibody and the additional receptor ECD is fused to the light chain of the antibody. Alternatively, BTLA ECD is fused to light chain and additional receptor ECD is fused to heavy chain. In some embodiments, the additional receptor ECD is the ECD of a T cell co-inhibitory molecule. In one embodiment, the T cell co-inhibitory molecule is PD-1. In some embodiments, the fusion protein is selected from: anti-IL6R-BTLA-PD1 (e.g., SEQ ID NOs: 313, 314). In another embodiment, the T cell co-inhibitory molecule is TIM3. In some embodiments, the fusion protein is selected from: anti-IL6R-BTLA-TIM3 (e.g., SEQ ID NOs: 313, 317). In another aspect, the additional receptor ECD is SIRPa ECD. In some embodiments, the fusion protein is selected from: anti-IL6R-BTLA-SIRPa (e.g., SEQ ID NOs: 313, 316). In another aspect, the additional receptor ECD is SIGLEC10 ECD. In some embodiments, the fusion protein is selected from: anti-IL6R-BTLA-SIGLEC10 (e.g., SEQ ID NOs: 313, 315). In another aspect, the additional receptor ECD is VEGFR ECD. In some embodiments, the fusion protein is selected from: anti-IL6R-VEGFR-BTLA (e.g., SEQ ID NOs: 324, 319).

In some embodiments, the fusion protein comprises BTLA ECD and an antibody that binds TGFb, TGFbR, or GARP. Exemplary embodiments of this fusion protein include anti-TGFb-BTLA (e.g., SEQ ID NOs: 393, 133), anti-TGFbR-BTLA, and anti-GARP-BTLA (e.g., SEQ ID NOs: 410, 49).

In some embodiments, the fusion protein further comprises an additional receptor ECD. Exemplary embodiments of this fusion protein include anti-TGFb-BTLA-TIM3 (e.g., SEQ ID NOs: 393, 397); anti-TGFb-BTLA-PD1 (e.g., SEQ ID NOs: 393, 394); anti-TGFb-BTLA-SIRPa (e.g., SEQ ID NOs: 393, 396); anti-TGFb-BTLA-SIGLEC10 (e.g., SEQ ID NOs: 393, 395).

In some embodiments, the fusion protein comprises BTLA ECD and IL-15. In some embodiments, the fusion protein is IL15-Fc-BTLA (e.g., SEQ ID NO: 574) or BTLA-Fc-IL15 (e.g., SEQ ID NO: 573). In other embodiments, the fusion protein is IL12-Fc-BTLA (e.g., SEQ ID NO: 572) or BTLA-Fc-IL12 (e.g., SEQ ID NO: 571). In other embodiments, the fusion protein comprises an antibody with BTLA ECD fused to heavy chain and IL-15 fused to light chain. In other embodiments, the fusion protein comprises an antibody with BTLA ECD fused to heavy chain and IL-12 fused to light chain.

In various embodiments, the fusion proteins of the invention counteract CD47/SIRPa in the tumor microenvironment. In some embodiments, the fusion proteins comprise a ligand-binding sequence of an extracellular domain of SIRPa (e.g., SIRPa ECD).

In one embodiment, the fusion protein of the invention is an ALT that comprises an antibody that binds CD47 or CD47 ligand (e.g. SIRPa). In one example, the ALT comprises an antibody that binds CD47 and interferes with its interaction with SIRPa. In one example, the ALT comprises an antibody that binds SIRPa and interferes with its interaction with CD47. In one aspect, the antibody is an antagonist that inhibits SIRPa intracellular ITIM or ITSM signaling, thereby promoting immune cell activation. In various examples, the antibody that binds CD47 or SIRPa is fused to one or more ligand traps. In one example the CD47 or SIRPa antibody is fused to a ligand-binding sequence of the extracellular domain of PD1 (PD1 ECD). In one example the CD47 or SIRPa antibody is fused to a ligand-binding sequence of the extracellular domain of TGFbR (TGFbRII ECD). In one example the CD47 or SIRPa antibody is fused to a ligand-binding sequence of the extracellular domain of TIM3 (TIM3 ECD). In one example the CD47 or SIRPa antibody is fused to a ligand-binding sequence of the extracellular domain of BTLA (BTLA ECD). In one example the CD47 or SIRPa antibody is fused to a ligand-binding sequence of the extracellular domain of SIGLEC10 (SIGLEC10 ECD). In one example the CD47 or SIRPa antibody is fused to a ligand-binding sequence of the extracellular domain of VEGFR (VEGFR ECD). In one embodiment, the CD47 or SIRPa binding antibody is fused to multiple ligand traps selected from the following: PD1 ECD, TGFbRII ECD, TIM3 ECD, BTLA ECD, VEGFR ECD, SIGLEC10 ECD. In one aspect, a ligand trap (LT1) is fused to the heavy chain, and a second ligand trap (LT2) is fused to the light chain. In another aspect, LT1 or LT2 functions to localize the fusion protein to the tumor microenvironment. In one

In various examples, the ALT comprises a CD47 or SIRPa binding antibody, wherein the heavy chain is fused to TGFbRII ECD and the light chain is fused to PD1 ECD, BTLA ECD, TIM3 ECD, or SIGLEC10 ECD. In various examples, the ALT comprises a CD47 or SIRPa binding antibody, wherein the heavy chain is fused to VEGFR ECD and the light chain is fused to PD1 ECD, BTLA ECD, TIM3 ECD, or SIGLEC10 ECD. In various examples, the ALT comprises a CD47 or SIRPa binding antibody, wherein the heavy chain is fused to PD1 ECD and the light chain is fused to BTLA ECD, TIM3 ECD, or SIGLEC10 ECD. In various examples, the ALT comprises a CD47 or SIRPa binding antibody, wherein the heavy chain is fused to BTLA ECD and the light chain is fused to PD1 ECD, TIM3 ECD, or SIGLEC10 ECD. In various examples, the ALT comprises a CD47 or SIRPa binding antibody, wherein the heavy chain is fused to TIM3 ECD and the light chain is fused to BTLA ECD, PD1 ECD, or SIGLEC10 ECD. In various examples, the ALT comprises a CD47 or SIRPa binding antibody, wherein the heavy chain is fused to SIGLEC10 ECD and the light chain is fused to BTLA ECD, TIM3 ECD, or PD1 ECD.

In one embodiment, the fusion proteins of the invention may comprise a ligand-binding sequence of an extracellular domain of SIRPa to interrupt the interaction of SIRPa and CD47. In one embodiment, the fusion protein comprises a targeting polypeptide and the SIRPa ECD is fused to the targeting polypeptide. In some embodiments, the targeting polypeptide is an antibody and SIRPa ECD is fused to the heavy chain of the antibody. In other embodiments, the targeting polypeptide is an antibody and SIRPa ECD is fused to the light chain of the antibody.

In some embodiments, the fusion protein of the invention comprises a targeting polypeptide that is an antibody. In one embodiment, the antibody is fused to SIRPa ECD and additional ligand traps selected from the following: PD1 ECD, TGFbRII ECD, BTLA ECD, TIM3 ECD, VEGFR ECD, SIGLEC10 ECD. In one aspect, a ligand trap (LT1) is fused to the heavy chain, and a second ligand trap (LT2) is fused to the light chain. In one aspect, the SIRPa ECD is fused to the heavy chain and another ligand trap is fused to the light chain. In one aspect, the SIRPa ECD is fused to the light chain and another ligand trap is fused to the heavy chain. In various examples, the ALT comprises an antibody, wherein the heavy chain is fused to TIM3 ECD and the light chain is fused to one of PD1 ECD, BTLA ECD, SIRPa ECD, or SIGLEC10 ECD. In various examples, the ALT comprises an antibody wherein the light chain is fused to TIM3 ECD and the heavy chain is fused to one of PD1 ECD, TGFbRII ECD, BTLA ECD, TIM3 ECD, VEGFR ECD, SIGLEC10 ECD.

In some embodiments, the fusion protein comprises SIRPa ECD and a polypeptide that inhibits a T cell co-inhibitory molecule. In specific embodiments, the fusion protein comprises SIRPa ECD and an antibody that binds and disables a T cell co-inhibitory molecule.

In some embodiments, the fusion protein comprises SIRPa ECD and a polypeptide that inhibits the interaction of TIGIT or PVRIG with PVRL2 or PVR. In some embodiments, this fusion protein comprises an antibody that binds TIGIT or PVRIG fused to SIRPa ECD. Exemplary embodiments of this fusion protein include anti-TIGIT-SIRPa (e.g., SEQ ID NOs: 477, 139) and anti-PVRIG-SIRPa.

In some embodiments, the fusion protein comprises SIRPa ECD and a polypeptide that inhibits the interaction of VISTA and VSIG8. In some embodiments, this fusion protein comprises an antibody that binds VISTA or VSIG8 fused to SIRPa ECD. Exemplary embodiments of this fusion protein include anti-VISTA-SIRPa and anti-VSIG8-SIRPa.

In some embodiments, the fusion protein comprises SIRPa ECD and a polypeptide that inhibits the interaction of PD-1 and PD-L1. In some embodiments, this fusion protein comprises an antibody that binds PD-1 or PD-L1 fused to SIRPa ECD. Exemplary embodiments of this fusion protein include anti-PD1-SIRPa (e.g., SEQ ID NOs: 455, 101) and anti-PDL1-SIRPa (e.g., SEQ ID NOs: 465, 109).

In some embodiments, the fusion protein comprises SIRPa ECD and a polypeptide that inhibits CTLA-4. In some embodiments, this fusion protein comprises an antibody that binds CTLA-4 fused to SIRPa ECD. Exemplary embodiments of this fusion protein include anti-CTLA4-SIRPa (e.g., SEQ ID NOs: 443, 28).

In some embodiments, the fusion protein comprises SIRPa ECD and a polypeptide that inhibits TIM-3. In some embodiments, this fusion protein comprises an antibody that binds TIM-3 fused to SIRPa ECD. Exemplary embodiments of this fusion protein include anti-TIM3-SIRPa (e.g., SEQ ID NOs: 489, 141).

In some embodiments, the fusion protein comprises SIRPa ECD, an antibody that binds and disables a T cell co-inhibitory molecule, and an additional receptor ECD. In one aspect, the SIRPa ECD is fused to the heavy chain of the antibody and the additional receptor ECD is fused to the light chain of the antibody. Alternatively, the SIRPa ECD is fused to the light chain and the additional receptor ECD is fused to the heavy chain

In other embodiments, the fusion protein comprises SIRPa ECD, an antibody that binds and disables a T cell co-inhibitory molecule, and SIGLEC10 ECD. Exemplary embodiments of this fusion protein include anti-TIGIT-SIRPa-SIGLEC10 (e.g., SEQ ID NOs: 477, 471); anti-PD1-SIRPa-SIGLEC10 (e.g., SEQ ID NOs: 455, 449); anti-PDL1-SIRPa-SIGLEC10 (e.g., SEQ ID NOs: 465, 460); anti-TIM3-SIRPa-SIGLEC10 (e.g., SEQ ID NOs: 489, 483); anti-CTLA4-SIRPa-SIGLEC10 (e.g., SEQ ID NOs: 443, 437).

In some embodiments, the fusion protein comprises an antibody that binds a T cell co-stimulatory molecule fused to SIRPa. This antibody is preferably an agonist of the T cell co-stimulatory molecule. In some embodiments, this fusion protein is selected from: anti-ICOS-SIRPa (e.g., SEQ ID NOs: 525, 59); anti-OX40-SIRPa (e.g., SEQ ID NOs: 513, 97); anti-41BB-SIRPa (e.g., SEQ ID NOs: 501, 2).

In a further aspect, the fusion protein comprises an antibody that binds a T cell-costimulatory molecule, SIRPa ECD, and an additional receptor ECD. In one aspect, the SIRPa ECD is fused to the heavy chain of the antibody and the additional receptor ECD is fused to the light chain of the antibody. Alternatively, the SIRPa ECD is fused to the light chain of the antibody and the additional receptor ECD is fused to the heavy chain of the antibody. In some embodiments, the additional receptor ECD is the ECD of a T cell co-inhibitory molecule. In another aspect, the additional receptor ECD is SIGLEC10 ECD. In some embodiments, the fusion protein is selected from: anti-41BB—SIRPa-SIGLEC10 (e.g., SEQ ID NOs: 501, 495); anti-ICOS-SIRPa-SIGLEC10 (e.g., SEQ ID NOs: 525, 519); anti-OX40-SIRPa-SIGLEC10 (e.g., SEQ ID NOs: 513, 507).

In some embodiments, the fusion protein comprises an antibody, SIRPa ECD, and the ECD of a T cell co-stimulatory molecule. In one aspect, the SIRPa ECD is fused to heavy chain and the ECD of a T cell co-stimulatory molecule fused to light chain. Alternatively, the SIRPa ECD is fused to the light chain and the T cell co-stimulatory molecule ECD is fused to the heavy chain. In some embodiments, the fusion protein comprises SIRPa ECD and one of the following: OX40L, 41BBL, ICOSL.

In one embodiment, the fusion protein comprises SIRPa ECD and a polypeptide that binds an ectonucleotidase. In a preferred embodiment, the ectonucleotidase is either CD39 or CD73. In some embodiments, the fusion protein is an antibody that binds CD39 or CD73 fused to SIRPa ECD; for example: anti-CD39-SIRPa (e.g., SEQ ID NOs: 431, 18) or anti-CD73-SIRPa (e.g., SEQ ID NOs: 424, 24).

In some embodiments, the fusion protein comprises SIRPa ECD, an antibody that binds CD39 or CD73, and an additional receptor ECD. In some embodiments, the SIRPa ECD is fused to the heavy chain of the antibody and the additional receptor ECD is fused to the light chain of the antibody. Alternatively, the SIRPa ECD is fused to the light chain of the antibody and the additional receptor ECD is fused to the heavy chain of the antibody.

In some embodiments, the fusion protein comprises a tumor-targeted antibody and SIRPa ECD. In some embodiments, this tumor targeted-antibody binds a tumor growth factor or growth factor receptor. In other embodiments, this tumor targeted antibody binds a tumor cell surface molecule. In some embodiments, this fusion protein is selected from the following: anti-EGFR-SIRPa (e.g., SEQ ID NOs: 228, 43), anti-HER2-SIRPa (e.g., SEQ ID NOs: 252, 55), anti-EGFRvIII-SIRPa (e.g., SEQ ID NOs: 240, 47), anti-uPAR-SIRPa (e.g., SEQ ID NOs: 271, 162), anti-PSMA-SIRPa (e.g., SEQ ID NOs: 278, 121), anti-nectin-4-SIRPa.

In a further embodiment, the fusion protein comprises a tumor-targeted antibody, SIRPa ECD, and an additional receptor ECD. In one embodiment, the SIRPa ECD is fused to the heavy chain of the antibody and the additional receptor ECD is fused to the light chain of the antibody. Alternatively, the SIRPa ECD is fused to the light chain and the additional receptor ECD is fused to the heavy chain. In some embodiments, the additional receptor ECD is the ECD of a T cell co-inhibitory molecule. In another embodiment, the additional receptor ECD is SIGLEC10 ECD. In some embodiments, the fusion protein is selected from: anti-HER2-SIRPa-SIGLEC10 (e.g., SEQ ID NOs: 252, 246); anti-nectin4-SIRPa-SIGLEC10 (e.g., SEQ ID NOs: 264, 258); anti-EGFR—SIRPa-SIGLEC10 (e.g., SEQ ID NOs: 228, 222); anti-EGFRvIII—SIRPa-SIGLEC10 (e.g., SEQ ID NOs: 240, 234).

In some embodiments, the fusion protein comprises SIRPa ECD and a polypeptide that inhibits VEGF/VEGFR signaling.

In some embodiments, the fusion protein comprises SIRPa ECD and anti-VEGFR mAb with SIRPa ECD fused to either the heavy chain or light chain of the antibody. In some embodiments, this fusion protein is anti-VEGFR-SIRPa (e.g., SEQ ID NOs: 380, 148). In a further embodiment, the fusion protein may additionally comprise a receptor ECD fused to the other chain of the antibody. In some embodiments, the fusion protein is selected from anti-VEGFR-SIRPa-PD1 (e.g., SEQ ID NOs: 380, 373); anti-VEGFR-SIRPa-TIM3 (e.g., SEQ ID NOs: 380, 376); anti-VEGFR-SIRPa-BTLA (e.g., SEQ ID NOs: 380, 378); anti-VEGFR-SIRPa-SIGLEC10 (e.g., SEQ ID NOs: 380, 374).

In some embodiments, the fusion protein comprises SIRPa ECD and anti-VEGF mAb with SIRPa ECD fused to either the heavy chain or light chain of the antibody. In some embodiments, this fusion protein is anti-VEGF-SIRPa (e.g., SEQ ID NOs: 369, 32). In a further embodiment, the fusion protein may additionally comprise a receptor ECD fused to the other chain of the antibody. In some embodiments, the fusion protein is selected from anti-VEGF-SIRPa-BTLA (e.g., SEQ ID NOs: 369, 367); anti-VEGF-SIRPa-SIGLEC10 (e.g., SEQ ID NOs: 369, 363); anti-VEGF-SIRPa-TIM3 (e.g., SEQ ID NOs: 369, 365); anti-VEGF-SIRPa-PD1 (e.g., SEQ ID NOs: 369, 362)

In some embodiments, the fusion protein comprises an antibody that binds IL-17 or IL-17R and SIRPa ECD. In some embodiments, this fusion protein is selected from the following: anti-IL17-SIRPa, anti-IL17R-SIRPa (e.g., SEQ ID NOs: 333, 63).

In a further embodiment, the fusion protein comprises an antibody that binds IL-17 or IL-17R, SIRPa ECD, and an additional receptor ECD. In one embodiment, SIRPa ECD is fused to the heavy chain of the antibody and the additional receptor ECD is fused to the light chain of the antibody. Alternatively, SIRPa ECD is fused to light chain and additional receptor ECD is fused to heavy chain. In some embodiments, the additional receptor ECD is the ECD of a T cell co-inhibitory molecule. In another aspect, the additional receptor ECD is SIGLEC10 ECD. In some embodiments, the fusion protein is selected from: anti-IL17R-SIRPa-SIGLEC10 (e.g., SEQ ID NOs: 333, 327).

In some embodiments, the fusion protein comprises an antibody that binds IL-23 or IL-23R and SIRPa ECD. If the antibody binds IL-23, it is preferred that the antibody bind the p19 subunit of IL-23 that is not shared with IL-12. In some embodiments, this fusion protein is selected from the following: anti-IL23-SIRPa (e.g., SEQ ID NOs: 345, 75), anti-IL23R-SIRPa.

In a further embodiment, the fusion protein comprises an antibody that binds IL-23 or IL-23R, SIRPa ECD, and an additional receptor ECD. In one embodiment, SIRPa ECD is fused to the heavy chain of the antibody and the additional receptor ECD is fused to the light chain of the antibody. Alternatively, SIRPa ECD is fused to light chain and additional receptor ECD is fused to heavy chain. In some embodiments, the additional receptor ECD is the ECD of a T cell co-inhibitory molecule. In another aspect, the additional receptor ECD is SIGLEC10 ECD. In some embodiments, the fusion protein is selected from: anti-IL23-SIRPa-SIGLEC10 (e.g., SEQ ID NOs: 345, 339).

In some embodiments, the fusion protein comprises an antibody that binds IL-6 or IL-6R and SIRPa ECD. In some embodiments, this fusion protein is selected from the following: anti-IL6-SIRPa, anti-IL6R-SIRPa (e.g., SEQ ID NOs: 321, 79).

In some embodiments, the fusion protein comprises an antibody that binds IL-6 or IL-6R, SIRPa ECD, and an additional receptor ECD. In one embodiment, SIRPa ECD is fused to the heavy chain of the antibody and the additional receptor ECD is fused to the light chain of the antibody. Alternatively, SIRPa ECD is fused to light chain and additional receptor ECD is fused to heavy chain. In some embodiments, the additional receptor ECD is the ECD of a T cell co-inhibitory molecule. In another aspect, the additional receptor ECD is SIGLEC10 ECD. In some embodiments, the fusion protein is selected from: anti-IL6R-SIRPa-SIGLEC10 (e.g., SEQ ID NOs: 321, 315).

In some embodiments, the fusion protein comprises SIRPa ECD and an antibody that binds TGFb, TGFbR, or GARP. Exemplary embodiments of this fusion protein include anti-TGFb-SIRPa (e.g., SEQ ID NOs: 401, 133), anti-TGFbR-SIRPa, and anti-GARP-SIRPa (e.g., SEQ ID NOs: 413, 49).

In some embodiments, the fusion protein further comprises an additional receptor ECD. Exemplary embodiments of this fusion protein include anti-TGFb-SIRPa-BTLA (e.g., SEQ ID NOs: 401, 399); anti-TGFb-SIRPa-TIM3 (e.g., SEQ ID NOs: 401, 397); anti-TGFb-SIRPa-PD1 (e.g., SEQ ID NOs: 401, 394); anti-TGFb-SIRPa-SIGLEC10 (e.g., SEQ ID NOs: 401, 395).

In some embodiments, the fusion protein comprises SIRPa ECD and IL-15. In some embodiments, the fusion protein is IL15-Fc-SIRPa (e.g., SEQ ID NO: 586) or SIRPa-Fc-IL15 (e.g., SEQ ID NO: 585). In other embodiments, the fusion protein is IL12-Fc-SIRPa (e.g., SEQ ID NO: 584) or SIRPa-Fc-IL12 (e.g., SEQ ID NO: 583). In other embodiments, the fusion protein comprises an antibody with SIRPa ECD fused to heavy chain and IL-15 fused to light chain. In other embodiments, the fusion protein comprises an antibody with SIRPa ECD fused to heavy chain and IL-12 fused to light chain.

In various embodiments, the fusion proteins of the invention counteract SIGLEC10/CD24 in the tumor microenvironment. In some embodiments, the fusion proteins comprise a ligand-binding sequence of an extracellular domain of SIGLEC10 (e.g., SIGLEC10 ECD).

In one embodiment, the fusion protein of the invention is an ALT that comprises an antibody that binds SIGLEC10 or CD24. In one example, the ALT comprises an antibody that binds CD24 and interferes with its interaction with SIGLEC10. In one example, the ALT comprises an antibody that binds SIGLEC10 and interferes with its interaction with CD24. In one aspect, the antibody is an antagonist that inhibits SIGLEC10 intracellular ITIM or ITSM signaling, thereby promoting immune cell activation. In various examples, the antibody that binds CD24 or SIGLEC10 is fused to one or more ligand traps. In one example the CD24 or SIGLEC10 antibody is fused to a ligand-binding sequence of the extracellular domain of PD1 (PD1 ECD). In one example the CD24 or SIGLEC10 antibody is fused to a ligand-binding sequence of the extracellular domain of TGFbR (TGFbRII ECD). In one example the CD24 or SIGLEC10 antibody is fused to a ligand-binding sequence of the extracellular domain of TIM3 (TIM3 ECD). In one example the CD24 or SIGLEC10 antibody is fused to a ligand-binding sequence of the extracellular domain of BTLA (BTLA ECD). In one example the CD24 or SIGLEC10 antibody is fused to a ligand-binding sequence of the extracellular domain of SIRPa (SIRPa ECD). In one example the CD24 or SIGLEC10 antibody is fused to a ligand-binding sequence of the extracellular domain of VEGFR (VEGFR ECD). In one embodiment, the CD24 or SIGLEC10 binding antibody is fused to multiple ligand traps selected from the following: PD1 ECD, TGFbRII ECD, TIM3 ECD, BTLA ECD, VEGFR ECD, SIRPa ECD. In one aspect, a ligand trap (LT1) is fused to the heavy chain, and a second ligand trap (LT2) is fused to the light chain.

In various examples, the ALT comprises a CD24 or SIGLEC10 binding antibody, wherein the heavy chain is fused to TGFbRII ECD and the light chain is fused to PD1 ECD, BTLA ECD, TIM3 ECD, or SIRPa ECD. In various examples, the ALT comprises a CD24 or SIGLEC10 binding antibody, wherein the heavy chain is fused to VEGFR ECD and the light chain is fused to PD1 ECD, BTLA ECD, TIM3 ECD, or SIRPa ECD. In various examples, the ALT comprises a CD24 or SIGLEC10 binding antibody, wherein the heavy chain is fused to PD1 ECD and the light chain is fused to BTLA ECD, TIM3 ECD, or SIRPa ECD. In various examples, the ALT comprises a CD24 or SIGLEC10 binding antibody, wherein the heavy chain is fused to BTLA ECD and the light chain is fused to PD1 ECD, TIM3 ECD, or SIRPa ECD. In various examples, the ALT comprises a CD24 or SIGLEC10 binding antibody, wherein the heavy chain is fused to TIM3 ECD and the light chain is fused to BTLA ECD, PD1 ECD, or SIRPa ECD. In various examples, the ALT comprises a CD24 or SIGLEC10 binding antibody, wherein the heavy chain is fused to SIRPa ECD and the light chain is fused to BTLA ECD, TIM3 ECD, or PD1 ECD.

In one embodiment, the fusion proteins of the invention may comprise a ligand-binding sequence of an extracellular domain of SIGLEC10 to interrupt the interaction of SIGLEC10 and CD24. In one embodiment, the fusion protein comprises a targeting polypeptide and the SIGLEC10 ECD is fused to the targeting polypeptide. In some embodiments, the targeting polypeptide is an antibody and SIGLEC10 ECD is fused to the heavy chain of the antibody. In other embodiments, the targeting polypeptide is an antibody and SIGLEC10 ECD is fused to the light chain of the antibody.

In some embodiments, the fusion protein of the invention comprises a targeting polypeptide that is an antibody. In one embodiment, the antibody is fused to SIGLEC10 ECD and additional ligand traps selected from the following: PD1 ECD, TGFbRII ECD, BTLA ECD, TIM3 ECD, VEGFR ECD, SIRPa ECD. In one aspect, a ligand trap (LT1) is fused to the heavy chain, and a second ligand trap (LT2) is fused to the light chain. In one aspect, the SIGLEC10 ECD is fused to the heavy chain and another ligand trap is fused to the light chain. In one aspect, the SIGLEC10 ECD is fused to the light chain and another ligand trap is fused to the heavy chain. In various examples, the ALT comprises an antibody, wherein the heavy chain is fused to TIM3 ECD and the light chain is fused to one of PD1 ECD, BTLA ECD, SIGLEC10 ECD, or SIRPa ECD. In various examples, the ALT comprises an antibody wherein the light chain is fused to TIM3 ECD and the heavy chain is fused to one of PD1 ECD, TGFbRII ECD, BTLA ECD, TIM3 ECD, VEGFR ECD, SIRPa ECD.

In some embodiments, the fusion protein comprises SIGLEC10 ECD and a polypeptide that inhibits the interaction between CD47 and SIRPa. In one embodiment, this fusion protein comprises SIGLEC10 ECD and a polypeptide that binds CD47. In one embodiment, this fusion protein is anti-CD47 mAb fused to SIGLEC10 ECD (anti-CD47-SIGLEC10 (e.g., SEQ ID NOs: 389, 22)). In another embodiment, this fusion protein comprises SIRPa ECD and SIGLEC10 ECD. In one embodiment, this fusion protein is SIRPa-Fc-SIGLEC10 (e.g., SEQ ID NO: 549) or SIGLEC10-Fc-SIRPa (e.g., SEQ ID NO: 543).

In a further aspect, the fusion protein comprises SIGLEC10 ECD, a polypeptide that inhibits the interaction between CD47 and SIRPa, and another polypeptide that inhibits a T cell co-inhibitory molecule. In some embodiments, this fusion protein comprises anti-CD47 mAb with SIGLEC10 ECD fused to the heavy chain or light chain; and a T cell co-inhibitory molecule ECD fused to the other chain. In a particular embodiment, this fusion protein is anti-CD47-SIGLEC10-PD1 (e.g., SEQ ID NOs: 389, 384). In another embodiment, this fusion protein is anti-CD47-SIGLEC10-TIM3 (e.g., SEQ ID NOs: 389, 386).

In another aspect, the fusion protein comprises SIGLEC10 ECD, anti-CD47 mAb, and VEGFR ECD. In some embodiments, VEGFR ECD is fused to heavy chain of anti-CD47 mAb and SIGLEC10 ECD is fused to light chain of anti-CD47 mAb. In one embodiment, this fusion protein is anti-CD47-VEGFR-SIGLEC10 (e.g., SEQ ID NOs: 392, 385).

In another aspect, the fusion protein comprises SIGLEC10 ECD, SIRPa ECD, and an antibody with a heavy chain and light chain. In one aspect, the SIGLEC10 ECD is fused to the heavy chain of the antibody and the SIRPa ECD is fused to the light chain of the antibody. In another aspect, the SIGLEC10 ECD is fused to the light chain of the antibody and the SIRPa ECD is fused to the heavy chain of the antibody. In some embodiments, the antibody of said fusion protein binds a T cell co-inhibitory molecule as an antagonist. Exemplary embodiments of such fusion proteins include anti-CTLA4-SIGLEC10-SIRPa (e.g., SEQ ID NOs: 442, 438), anti-PD1-SIGLEC10-SIRPa (e.g., SEQ ID NOs: 454, 450), and anti-PDL1-SIGLEC10-SIRPa (e.g., SEQ ID NOs: 463, 461). In other embodiments, the antibody of said fusion protein binds a T cell co-stimulatory molecule as an agonist. Exemplary embodiments of this fusion protein include anti-OX40-SIGLEC10-SIRPa (e.g., SEQ ID NOs: 512, 508), anti-41BB-SIGLEC10-SIRPa (e.g., SEQ ID NOs: 500, 496), and anti-CD40-SIGLEC10-SIRPa. In other embodiments, the antibody of said fusion protein is a tumor-targeted antibody. In some embodiments, this tumor targeted-antibody binds a tumor growth factor or growth factor receptor. In other embodiments, this tumor targeted antibody binds a tumor cell surface molecule. Exemplary embodiments of this fusion protein include anti-EGFR-SIGLEC10-SIRPa (e.g., SEQ ID NOs: 227, 223), anti-HER2-SIGLEC10-SIRPa (e.g., SEQ ID NOs: 251, 247), anti-EGFRvIII-SIGLEC10-SIRPa (e.g., SEQ ID NOs: 239, 235), anti-uPAR-SIGLEC10-SIRPa, and anti-PSMA-SIGLEC10-SIRPa.

In other embodiments, the antibody of said fusion protein binds a member of the TGFb pathway. In some embodiments, this antibody binds TGFb, TGFbR, or GARP. Exemplary embodiments of this fusion protein include anti-TGFb-SIGLEC10-SIRPa (e.g., SEQ ID NOs: 400, 396), anti-TGFbR-SIGLEC10-SIRPa, and anti-GARP-SIGLEC10-SIRPa.

In other embodiments, the antibody of said fusion protein binds VEGF or VEGFR. Exemplary embodiments of this fusion protein include anti-VEGF-SIGLEC10-SIRPa (e.g., SEQ ID NOs: 368, 364) and anti-VEGFR-SIGLEC10-SIRPa (e.g., SEQ ID NOs: 379, 375).

In some embodiments, the fusion protein comprises SIGLEC10 ECD and a polypeptide that inhibits a T cell co-inhibitory molecule. In specific embodiments, the fusion protein comprises SIGLEC10 ECD and an antibody that binds and disables a T cell co-inhibitory molecule.

In some embodiments, the fusion protein comprises SIGLEC10 ECD and a polypeptide that inhibits the interaction of TIGIT or PVRIG with PVRL2 or PVR. In some embodiments, this fusion protein comprises an antibody that binds TIGIT or PVRIG fused to SIGLEC10 ECD. Exemplary embodiments of this fusion protein include anti-TIGIT-SIGLEC10 (e.g., SEQ ID NOs: 476, 139) and anti-PVRIG-SIGLEC10.

In some embodiments, the fusion protein comprises SIGLEC10 ECD and a polypeptide that inhibits the interaction of VISTA and VSIG8. In some embodiments, this fusion protein comprises an antibody that binds VISTA or VSIG8 fused to SIGLEC10 ECD. Exemplary embodiments of this fusion protein include anti-VISTA-SIGLEC10 and anti-VSIG8-SIGLEC10.

In some embodiments, the fusion protein comprises SIGLEC10 ECD and a polypeptide that inhibits the interaction of PD-1 and PD-L1. In some embodiments, this fusion protein comprises an antibody that binds PD-1 or PD-L1 fused to SIGLEC10 ECD. Exemplary embodiments of this fusion protein include anti-PD1-SIGLEC10 (e.g., SEQ ID NOs: 454, 101) and anti-PDL1-SIGLEC10 (e.g., SEQ ID NOs: 463, 109).

In some embodiments, the fusion protein comprises SIGLEC10 ECD and a polypeptide that inhibits CTLA-4. In some embodiments, this fusion protein comprises an antibody that binds CTLA-4 fused to SIGLEC10 ECD. Exemplary embodiments of this fusion protein include anti-CTLA4-SIGLEC10 (e.g., SEQ ID NOs: 442, 28).

In some embodiments, the fusion protein comprises SIGLEC10 ECD and a polypeptide that inhibits TIM-3. In some embodiments, this fusion protein comprises an antibody that binds TIM-3 fused to SIGLEC10 ECD. Exemplary embodiments of this fusion protein include anti-TIM3-SIGLEC10 (e.g., SEQ ID NOs: 488, 141).

In some embodiments, the fusion protein comprises SIGLEC10 ECD, an antibody that binds and disables a T cell co-inhibitory molecule, and an additional receptor ECD. In one aspect, the SIGLEC10 ECD is fused to the heavy chain of the antibody and the additional receptor ECD is fused to the light chain of the antibody. Alternatively, the SIGLEC10 ECD is fused to the light chain and the additional receptor ECD is fused to the heavy chain.

Fusion proteins comprising SIGLEC10 ECD and antibody that binds a T cell co-stimulatory molecule as an agonist

In some embodiments, the fusion protein comprises an antibody that binds a T cell co-stimulatory molecule fused to SIGLEC10. This antibody is preferably an agonist of the T cell co-stimulatory molecule. In some embodiments, this fusion protein is selected from: anti-41BB-SIGLEC10 (e.g., SEQ ID NOs: 500, 2); anti-OX40-SIGLEC10 (e.g., SEQ ID NOs: 512, 97); anti-ICOS-SIGLEC10 (e.g., SEQ ID NOs: 524, 59).

In a further aspect, the fusion protein comprises an antibody that binds a T cell-costimulatory molecule, SIGLEC10 ECD, and an additional receptor ECD. In one aspect, the SIGLEC10 ECD is fused to the heavy chain of the antibody and the additional receptor ECD is fused to the light chain of the antibody. Alternatively, the SIGLEC10 ECD is fused to the light chain of the antibody and the additional receptor ECD is fused to the heavy chain of the antibody. In some embodiments, the additional receptor ECD is the ECD of a T cell co-inhibitory molecule.

In some embodiments, the fusion protein comprises an antibody, SIGLEC10 ECD, and the ECD of a T cell co-stimulatory molecule. In one aspect, the SIGLEC10 ECD is fused to heavy chain and the ECD of a T cell co-stimulatory molecule fused to light chain. Alternatively, the SIGLEC10 ECD is fused to the light chain and the T cell co-stimulatory molecule ECD is fused to the heavy chain. In some embodiments, the fusion protein comprises SIGLEC10 ECD and one of the following: OX40L, 41BBL, ICOSL.

In one embodiment, the fusion protein comprises SIGLEC10 ECD and a polypeptide that binds an ectonucleotidase. In a preferred embodiment, the ectonucleotidase is either CD39 or CD73. In some embodiments, the fusion protein is an antibody that binds CD39 or CD73 fused to SIGLEC10 ECD; for example: anti-CD39-SIGLEC10 (e.g., SEQ ID NOs: 430, 18) or anti-CD73-SIGLEC10 (e.g., SEQ ID NOs: 423, 24).

In some embodiments, the fusion protein comprises SIGLEC10 ECD, an antibody that binds CD39 or CD73, and an additional receptor ECD. In some embodiments, the SIGLEC10 ECD is fused to the heavy chain of the antibody and the additional receptor ECD is fused to the light chain of the antibody. Alternatively, the SIGLEC10 ECD is fused to the light chain of the antibody and the additional receptor ECD is fused to the heavy chain of the antibody.

In some embodiments, the fusion protein comprises a tumor-targeted antibody and SIGLEC10 ECD. In some embodiments, this tumor targeted-antibody binds a tumor growth factor or growth factor receptor. In other embodiments, this tumor targeted antibody binds a tumor cell surface molecule. In some embodiments, this fusion protein is selected from the following: anti-EGFR-SIGLEC10 (e.g., SEQ ID NOs: 227, 43), anti-HER2-SIGLEC10 (e.g., SEQ ID NOs: 251, 55), anti-EGFRvIII-SIGLEC10 (e.g., SEQ ID NOs: 239, 47), anti-uPAR-SIGLEC10 (e.g., SEQ ID NOs: 270, 162), anti-PSMA-SIGLEC10 (e.g., SEQ ID NOs: 277, 121), anti-nectin-4-SIGLEC10.

In a further embodiment, the fusion protein comprises a tumor-targeted antibody, SIGLEC10 ECD, and an additional receptor ECD. In one embodiment, the SIGLEC10 ECD is fused to the heavy chain of the antibody and the additional receptor ECD is fused to the light chain of the antibody. Alternatively, the SIGLEC10 ECD is fused to the light chain and the additional receptor ECD is fused to the heavy chain.

In some embodiments, the fusion protein comprises SIGLEC10 ECD and a polypeptide that inhibits VEGF/VEGFR signaling.

In some embodiments, the fusion protein comprises SIGLEC10 ECD and anti-VEGFR mAb with SIGLEC10 ECD fused to either the heavy chain or light chain of the antibody. In some embodiments, this fusion protein is anti-VEGFR-SIGLEC10 (e.g., SEQ ID NOs: 379, 148). In a further embodiment, the fusion protein may additionally comprise a receptor ECD fused to the other chain of the antibody.

In some embodiments, the fusion protein comprises SIGLEC10 ECD and anti-VEGF mAb with SIGLEC10 ECD fused to either the heavy chain or light chain of the antibody. In some embodiments, this fusion protein is anti-VEGF-SIGLEC10 (e.g., SEQ ID NOs: 368, 32). In a further embodiment, the fusion protein may additionally comprise a receptor ECD fused to the other chain of the antibody.

In other embodiments, the fusion protein comprises SIGLEC10 ECD and VEGFR ECD. In one embodiment, this fusion protein is SIGLEC10-Fc-VEGFR (e.g., SEQ ID NO: 546). In another embodiment, this fusion protein is VEGFR-Fc-SIGLEC10 (e.g., SEQ ID NO: 567).

In some embodiments, the fusion protein comprises an antibody that binds IL-17 or IL-17R and SIGLEC10 ECD. In some embodiments, this fusion protein is selected from the following: anti-IL17-SIGLEC10, anti-IL17R-SIGLEC10 (e.g., SEQ ID NOs: 332, 63).

In a further embodiment, the fusion protein comprises an antibody that binds IL-17 or IL-17R, SIGLEC10 ECD, and an additional receptor ECD. In one embodiment, SIGLEC10 ECD is fused to the heavy chain of the antibody and the additional receptor ECD is fused to the light chain of the antibody. Alternatively, SIGLEC10 ECD is fused to light chain and additional receptor ECD is fused to heavy chain. In some embodiments, the additional receptor ECD is the ECD of a T cell co-inhibitory molecule.

In some embodiments, the fusion protein comprises an antibody that binds IL-23 or IL-23R and SIGLEC10 ECD. If the antibody binds IL-23, it is preferred that the antibody bind the p19 subunit of IL-23 that is not shared with IL-12. In some embodiments, this fusion protein is selected from the following: anti-IL23-SIGLEC10 (e.g., SEQ ID NOs: 344, 75), anti-IL23R-SIGLEC10.

In a further embodiment, the fusion protein comprises an antibody that binds IL-23 or IL-23R, SIGLEC10 ECD, and an additional receptor ECD. In one embodiment, SIGLEC10 ECD is fused to the heavy chain of the antibody and the additional receptor ECD is fused to the light chain of the antibody. Alternatively, SIGLEC10 ECD is fused to light chain and additional receptor ECD is fused to heavy chain. In some embodiments, the additional receptor ECD is the ECD of a T cell co-inhibitory molecule.

In some embodiments, the fusion protein comprises an antibody that binds IL-6 or IL-6R and SIGLEC10 ECD. In some embodiments, this fusion protein is selected from the following: anti-IL6-SIGLEC10, anti-IL6R-SIGLEC10 (e.g., SEQ ID NOs: 320, 79).

In a further embodiment, the fusion protein comprises an antibody that binds IL-6 or IL-6R, SIGLEC10 ECD, and an additional receptor ECD. In one embodiment, SIGLEC10 ECD is fused to the heavy chain of the antibody and the additional receptor ECD is fused to the light chain of the antibody. Alternatively, SIGLEC10 ECD is fused to light chain and additional receptor ECD is fused to heavy chain. In some embodiments, the additional receptor ECD is the ECD of a T cell co-inhibitory molecule.

In some embodiments, the fusion protein comprises SIGLEC10 ECD and an antibody that binds TGFb, TGFbR, or GARP. Exemplary embodiments of this fusion protein include anti-TGFb-SIGLEC10 (e.g., SEQ ID NOs: 400, 133), anti-TGFbR-SIGLEC10, and anti-GARP-SIGLEC10 (e.g., SEQ ID NOs: 412, 49). In some embodiments, the fusion protein further comprises an additional receptor ECD.

In some embodiments, the fusion protein comprises SIGLEC10 ECD and IL-15. In some embodiments, the fusion protein is IL15-Fc-SIGLEC10 (e.g., SEQ ID NO: 582) or SIGLEC10-Fc-IL15 (e.g., SEQ ID NO: 581). In other embodiments, the fusion protein is IL12-Fc-SIGLEC10 (e.g., SEQ ID NO: 580) or SIGLEC10-Fc-IL12 (e.g., SEQ ID NO: 579). In other embodiments, the fusion protein comprises an antibody with SIGLEC10 ECD fused to heavy chain and IL-15 fused to light chain. In other embodiments, the fusion protein comprises an antibody with SIGLEC10 ECD fused to heavy chain and IL-12 fused to light chain.

In some embodiments, this invention covers fusion proteins comprising two different extracellular domains (ECDs).

In some embodiments, ECD #1 is a ligand-binding fragment of PD1 ECD. In some embodiments, ECD #2 is a ligand-binding fragment of a T cell co-inhibitory molecule. In some embodiments, ECD #2 is BTLA ECD or TIM3 ECD. In other embodiments, ECD #2 is VEGFR ECD. In other embodiments, ECD #2 is TGFbRII ECD. In other embodiments, ECD #2 is a cytokine receptor ECD. In other embodiments, ECD #2 is the ECD of a receptor whose ligation inhibits phagocytosis. In some embodiments, ECD #2 is SIRPa ECD or SIGLEC10 ECD.

In some embodiments, ECD #1 is a ligand-binding fragment of BTLA ECD. In some embodiments, ECD #2 is a ligand-binding fragment of a T cell co-inhibitory molecule. In some embodiments, ECD #2 is TIM3 ECD. In other embodiments, ECD #2 is VEGFR ECD. In other embodiments, ECD #2 is TGFbRII ECD. In other embodiments, ECD #2 is a cytokine receptor ECD. In other embodiments, ECD #2 is the ECD of a receptor whose ligation inhibits phagocytosis. In some embodiments, ECD #2 is SIRPa ECD or SIGLEC10 ECD.

In some embodiments, ECD #1 is a ligand-binding fragment of TIM3 ECD. In some embodiments, ECD #2 is a ligand-binding fragment of a T cell co-inhibitory molecule. In other embodiments, ECD #2 is VEGFR ECD. In other embodiments, ECD #2 is TGFbRII ECD. In other embodiments, ECD #2 is a cytokine receptor ECD. In other embodiments, ECD #2 is the ECD of a receptor whose ligation inhibits phagocytosis. In some embodiments, ECD #2 is SIRPa ECD or SIGLEC10 ECD.

In some embodiments, ECD #1 is a ligand-binding fragment of TGFbRII ECD. In some embodiments, ECD #2 is a ligand-binding fragment of a T cell co-inhibitory molecule. In other embodiments, ECD #2 is VEGFR ECD. In other embodiments, ECD #2 is a cytokine receptor ECD. In other embodiments, ECD #2 is the ECD of a receptor whose ligation inhibits phagocytosis. In some embodiments, ECD #2 is SIRPa ECD or SIGLEC10 ECD. In other embodiments, ECD #2 is the ECD of a T cell co-stimulatory ligand. In some embodiments, ECD #2 is one of OX40L, 41BBL, ICOSL, GITRL, LIGHT, CD70, CD40L, CD30L.

In some embodiments, ECD #1 is a ligand-binding fragment of VEGFR ECD. In some embodiments, ECD #2 is a ligand-binding fragment of a T cell co-inhibitory molecule. In other embodiments, ECD #2 is a cytokine receptor ECD. In other embodiments, ECD #2 is the ECD of a receptor whose ligation inhibits phagocytosis. In some embodiments, ECD #2 is SIRPa ECD or SIGLEC10 ECD. In other embodiments, ECD #2 is the ECD of a T cell co-stimulatory ligand. In some embodiments, ECD #2 is one of OX40L, 41BBL, ICOSL, GITRL, LIGHT, CD70, CD40L, CD30L.

In some embodiments, ECD #1 is a ligand-binding fragment of SIRPa ECD. In some embodiments, ECD #2 is a ligand-binding fragment of a T cell co-inhibitory molecule. In other embodiments, ECD #2 is a cytokine receptor ECD. In other embodiments, ECD #2 is the ECD of a receptor whose ligation inhibits phagocytosis. In some embodiments, ECD #2 is SIGLEC 10 ECD.

In some embodiments, ECD #1 is a ligand-binding fragment of SIGLEC10 ECD. In some embodiments, ECD #2 is a ligand-binding fragment of a T cell co-inhibitory molecule.

In other embodiments, ECD #2 is a cytokine receptor ECD. In other embodiments, ECD #2 is the ECD of a receptor whose ligation inhibits phagocytosis. In other embodiments, ECD #2 is the ECD of a T cell co-stimulatory ligand. In some embodiments, ECD #2 is one of OX40L, 41BBL, ICOSL, GITRL, LIGHT, CD70, CD40L, CD30L.

In various embodiments, the invention provides preferred fusion proteins for treatment of cancer by counteracting key ligands/receptors that promote angiogenesis and immune dysfunction in the TME.

In one embodiment, the invention describes VEGF or VEGFR binding fusion proteins (ALT or ECD-ECD comprising a VEGF antibody, VEGFR antibody, or VEGFR ECD). In various embodiments, the ALT or ECD-ECD enables preferential localization of VEGF blockade to the TME.

In one embodiment, the VEGF/VEGFR blocking fusion protein of the invention comprises a tumor targeted antibody fused to VEGFR ECD. In various examples, without being limited to them, the ALT comprises an antibody that targets a tumor-associated cell surface antigen or molecule wherein the heavy chain is fused to VEGFR ECD (e.g. anti-HER2-VEGFR ECD; anti-EGFRvIII-VEGFR ECD; anti-PSMA-VEGFR ECD; anti-CEA-VEGFR ECD). In another aspect, the light chain of the ALT may be additionally fused to a receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD.

In another aspect the ALT may be any antibody wherein the heavy chain is fused to VEGFR ECD, and the light chain is fused to another receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD.

In another embodiment, the VEGF/VEGFR blocking ALT comprises a VEGF or VEGFR binding antibody fused to a receptor ECD that binds a ligand expressed on tumor cells or the TME. In various examples the ALT contains PD1 ECD (e.g. anti-VEGF-PD1 ECD to bind PDL1/2 on tumor cells), or BTLA ECD (anti-VEGF-BTLA ECD to bind HVEM on tumor cells), or TIM3 ECD (anti-VEGF-TIM3 ECD to bind CEACAM1/5 on tumor cells) or SIRPa ECD (anti-VEGF-SIRPa ECD to bind CD47 on tumor cells) or SIGLEC10 ECD (anti-VEGF-SIGLEC10 ECD). In another aspect, the VEGF-binding ECD-ECD may comprise a polypeptide containing VEGFR ECD and a receptor ECD that binds a ligand expressed on tumor cells or the TME (e.g. PD1 ECD, BTLA ECD, TIM3 ECD, SIRPa ECD, SIGLEC10 ECD).

In another embodiment, the VEGF/VEGFR binding fusion protein of the invention simultaneously binds CD47. In one aspect, the ALT comprises a CD47 binding antibody fused to VEGFR ECD. In another aspect the heavy chain is fused to VEGFR ECD, and the light chain is fused to another receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD. In another aspect, the ALT comprises a VEGF binding antibody or VEGFR binding antibody fused to SIRPa ECD. In another aspect, the ECD-ECD comprises VEGFR ECD and SIRPa ECD (e.g. VEGFR ECD-Fc-SIRPa ECD; SIRPa ECD-Fc-VEGFR ECD).

In another embodiment, the VEGF/VEGFR binding fusion protein of the invention binds VEGF or VEGFR and disrupts a ligand/receptor that promotes TH17 cell differentiation/function or angiogenesis in the TME.

In another embodiment, the VEGF/VEGFR binding fusion protein of the invention binds VEGF and TGFb. In one aspect a TGFb binding antibody is fused to VEGFR ECD. In one aspect a GARP or LAP binding antibody is fused to VEGFR ECD. In another aspect, a VEGF or VEGFR binding antibody is fused to TGFbRII ECD. In another aspect, the light chain of the ALT may be additionally fused to a receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD. In another aspect, an ECD-ECD comprises VEGFR ECD and TGFbRII ECD (VEGFR ECD-Fc-TGFbRII ECD).

In another embodiment, the VEGF/VEGFR binding fusion protein of the invention simultaneously binds and disables a-IL17R or IL17. In one aspect, the ALT comprises an IL17R antibody fused to VEGFR ECD. In another aspect, a VEGF or VEFR antibody is fused to an anti-IL17 nanobody. In another aspect, VEFR ECD is fused to an anti-IL17 nanobody. In another embodiment, the VEGF/VEGFR binding ALT simultaneously binds and disables a-IL6R or IL6. In one aspect, the ALT comprises an IL6R or IL6 binding antibody fused to VEGFR ECD. In another embodiment, the VEGF/VEGFR binding ALT simultaneously binds and disables a-IL23 or IL23R. In one aspect, the ALT comprises an IL23 or IL23R binding antibody fused to VEGFR ECD. In another embodiment, the VEGF/VEGFR binding ALT simultaneously binds and disables a-IL1 or IL1R. In one aspect, the ALT comprises an IL1 or IL1R binding antibody fused to VEGFR ECD. In an additional aspect, the ALT may be any antibody (targeting IL17, IL17R, IL23, IL23R, IL1, IL1R, IL6, or IL6R) wherein the heavy chain is fused to VEGFR ECD, and the light chain is fused to another receptor ECD (e.g. SIRPa ECD; BTLA ECD, TIM3 ECD, or PD1 ECD).

In another preferred embodiment the VEGF/VEGFR binding fusion protein of the invention binds VEGF and a co-inhibitory molecule. In one aspect a the ALT comprises an antibody that binds a T cell co-inhibitory molecule (e.g. CTLA-4, PD-L1, TIM3, BTLA, HVEM, TIGIT, PVRIG, PVRL2, PVR, VISTA, VSIG8) is fused to VEGFR ECD (e.g. a-CTLA4-VEGFR, a-PDL1-VEGFR, a-PD1-VEGFR, a-TIM3-VEGFR, a-BTLA mAb-VEGFR, a-HVEM mAb-VEGFR). In an additional aspect, the ALT may comprise the antibody wherein the heavy chain is fused to VEGFR ECD, and the light chain is fused to another receptor ECD (e.g. SIRPacd; BTLA ECD, TIM3 ECD, or PD1 ECD). In another aspect the ECD-ECD comprises VEGFR ECD and another receptor that binds a ligand expressed on tumor cells (e.g. PDL1/2, HVEM, CEACAM1/5, SIRPa). Examples of such ECD-ECDs include (VEGFR ECD-Fc-BTLA ECD, VEGFR ECD-Fc-PD1 ECD, VEGFR ECD-Fc-TIM3 ECD, VEGFR ECD-Fc-SIRPa ECD, VEGFR ECD-Fc-SIGLEC10 ECD).

In another embodiment, the VEGF/VEGFR binding fusion protein of the invention binds VEGF and a T cell co-stimulatory molecule. In one aspect a the ALT comprises an antibody that binds a T cell co-stumulatory molecule (e.g. 41BB, OX40, CD40, ICOS, GITR, DNAM) is fused to VEGFR ECD (e.g. a-41BB-VEGFR ECD, a-OX40-VEGFR ECD, a-CD40-VEGFR ECD, a-ICOS-VEGFR ECD, a-GITR-VEGFR ECD, a-DNAM-VEGFR ECD). In another aspect, the ALT may be any antibody wherein the heavy chain is fused to VEGFR ECD, and the light chain is fused to another receptor ECD (e.g. SIRPa ECD; BTLA ECD, TIM3 ECD, or PD1 ECD, SIGLEC10 ECD). In another aspect the ECD-ECD comprises VEGFR ECD and another co-stimulatory ligand (e.g. 41BBL, OX40L, GITRL, CD40L, ICOSL). Examples of such ECD-ECDs include (VEGFR ECD-Fc-41BBL, VEGFR ECD-Fc-OX40L, VEGFR ECD-Fc-ICOSL).

In another embodiment, the VEGF/VEGFR binding fusion protein of the invention comprises an antibody that binds either CD73 or CD39, fused to VEGFR ECD (a-CD73-VEGFR ECD and a-CD39-VEGFR ECD). In another aspect, the ALT may be any antibody wherein the heavy chain is fused to VEGFR ECD, and the light chain is fused to another receptor ECD that binds a ligand expressed on tumor cells or the TME (e.g. SIRPa ECD; BTLA ECD, TIM3 ECD, or PD1 ECD, SIGLEC10 ECD).

In another aspect, the ALT may be any antibody wherein the heavy chain is fused to VEGFR ECD, and the light chain is fused to another receptor ECD that binds a ligand expressed on tumor cells or the TME (e.g. SIRPa ECD; BTLA ECD, TIM3 ECD, or PD1 ECD, SIGLEC10 ECD).

In one embodiment, the invention describes TGFb or TGFbR blocking fusion proteins. In various aspects, the fusion proteins are an ALT or ECD-ECD comprising an antibody that binds TGFb, TGFbR, GARP, or LAP, or comprising TGFbRII ECD. In various embodiments, the ALT or ECD-ECD enables preferential localization of TGFb blockade to the TME.

In one aspect, the TGFb/TGFBR blocking ALT comprises a tumor targeted antibody fused to TGFbRII ECD. In various examples, without being limited to them, the ALT comprises an antibody that targets a tumor-associated cell surface antigen or molecule fused to TGFbRII ECD (e.g. anti-HER2-TGFbRII ECD; anti-EGFR-TGFbRII ECD; anti-EGFRvIII-TGFbRII ECD; anti-PSMA-TGFbRII ECD; anti-CEA-TGFbRII ECD). In another aspect, the light chain of the ALT may be additionally fused to a receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD.

In another aspect the ALT may be any antibody wherein the heavy chain is fused to TGFbRII ECD, and the light chain is fused to another receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD.

In another aspect, the TGFb/TGFBR blocking ALT comprises a TGFb or TGFBR or GARP or LAP binding antibody fused to a receptor ECD that binds a ligand expressed on tumor cells or the TME. In various examples the ALT contains PD1 ECD to bind PDL1/2 on tumor cells (e.g. anti-TGFb-PD1 ECD; anti-LAP-PD1 ECD; anti-GARP-PD1 ECD), or BTLA ECD to bind HVEM on tumor cells (anti-TGFb-BTLA ECD; anti-LAP-BTLA ECD; anti-GARP-BTLA ECD), or TIM3 ECD to bind CEACAM1/5 on tumor cells (anti-TGFb-TIM3 ECD; anti-LAP-TIM3 ECD; anti-GARP-TIM3 ECD), or SIRPa ECD to bind CD47 on tumor cells (anti-TGFb-SIRP ECD; anti-LAP-SIRPa ECD; anti-GARP-SIRPa ECD)) or SIGLEC10 ECD. In another aspect, the ALT may be TGFb or TGFBR or GARP or LAP binding antibody wherein the heavy chain is fused to one receptor ECD, and the light chain is fused to another receptor ECD that binds a ligand expressed on tumor cells or the TME (e.g. SIRPa ECD; BTLA ECD, TIM3 ECD, or PD1 ECD, or SIGLEC10 ECD). In another embodiment, the TGFb-binding ECD-ECD may comprise a polypeptide containing TGFbRII ECD and a receptor ECD that binds a ligand expressed on tumor cells or the TME (e.g. PD1 ECD, BTLA ECD, TIM3 ECD, SIRPa ECD, SIGLEC10 ECD).

In another preferred embodiment the TGFb/TGFBR blocking ALT or ECD-ECD simultaneously binds CD47. In one aspect, the ALT comprises a CD47 binding antibody fused to TGFbRII ECD. In another aspect the heavy chain is fused to TGFbRII ECD, and the light chain is fused to another receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD. In another aspect, the ALT comprises a TGFb or TGFbR or LAP or GARP binding antibody fused to SIRPa ECD. In another aspect, the heavy chain is fused to SIRPa ECD, and the light chain is fused to another receptor ECD that binds a ligand expressed on tumor cells or the TME (e.g. BTLA ECD, TIM3 ECD, or PD1 ECD, or SIGLEC10 ECD). In another aspect, the ECD-ECD comprises TGFbRII ECD and SIRPa ECD (e.g. TGFbRII ECD-Fc-SIRPa ECD; SIRPa ECD-Fc-TGFbRII ECD).

In a preferred embodiment, the TGFb/TGFBR blocking ALT or ECD-ECD binds TGFb or TGFbR or LAP or GARP, and disrupts a ligand/receptor that promotes TH17 cell differentiation/function and angiogenesis in the TME.

In another embodiment, the TGFb/TGFBR blocking fusion protein of the invention binds VEGF and either TGFb, TGFbR, LAP or GARP. In one aspect a TGFb or TGFBR or GARP or LAP binding antibody is fused to VEGFR ECD. In another aspect, the ALT may be a TGFb or TGFBR or GARP or LAP binding antibody wherein the heavy chain is fused to VEGFR ECD, and the light chain is fused to another receptor ECD that binds a ligand expressed on tumor cells or the TME (e.g. SIRPa ECD; BTLA ECD, TIM3 ECD, or PD1 ECD or SIGLEC10 ECD). In another aspect, a VEGF or VEGFR binding antibody is fused to TGFbRII ECD. In another aspect, the ALT may be a VEGF or VEGFR antibody wherein the heavy chain is fused to TGFbRII ECD, and the light chain is fused to another receptor ECD that binds a ligand expressed on tumor cells or the TME (e.g. SIRPa ECD; BTLA ECD, TIM3 ECD, or PD1 ECD or SIGLEC10 ECD). In another aspect, an ECD-ECD comprises VEGFR ECD and TGFbRII ECD (VEGFR ECD-Fc-TGFbRII ECD).

In another embodiment, the TGFb/TGFBR blocking ALT simultaneously binds and disables a-IL17R or IL17. In one aspect, the ALT comprises an IL17R antibody fused to TGFbRII ECD. In another aspect, a TGFb or TGFBR or GARP or LAP binding antibody is fused to an anti-IL17 nanobody. In another aspect, TGFbRII ECD is fused to an anti-IL17 nanobody. In another embodiment, the TGFb/TGFBR blocking ALT simultaneously binds and disables a-IL6R or IL6. In one aspect, the ALT comprises an IL6R or IL6 binding antibody fused to TGFbRII ECD. In another embodiment, the TGFb/TGFBR blocking ALT simultaneously binds and disables a-IL23 or IL23R. In one aspect, the ALT comprises an IL23 or IL23R binding antibody fused to TGFbRII ECD. In another embodiment, the TGFb/TGFBR blocking ALT simultaneously binds and disables a-IL1 or IL1R. In one aspect, the ALT comprises an IL1 or IL1R binding antibody fused to TGFbRII ECD. In another aspect, the light chain of the ALT comprising an antibody (that targets IL17R, IL17, IL23R, IL23, IL6R, IL6, IL1R, or IL1) may be additionally fused to a receptor ECD that binds a ligand expressed on tumor cells or the TME. In various examples the additional receptor ECD fused to the light chain is PD1 ECD, TIM3 ECD, BTLA ECD, SIRPa ECD, or SIGLEC10 ECD.

In another preferred embodiment the TGFb/TGFBR binding ALT or ECD-ECD binds either TGFb or TGFbR or GARP or LAP, and a co-inhibitory molecule. In one aspect a the ALT comprises an antibody that binds a T cell co-inhibitory molecule (e.g. CTLA-4, PD-L1, TIM3, BTLA, HVEM, TIGIT, PVRIG, PVRL2, PVR, VISTA, VSIG8) is fused to TGFbRII ECD (e.g. a-CTLA4-TGFbRII, a-PDL1-TGFbRII, a-PD1-TGFbRII, a-TIM3-TGFbRII, a-BTLA mAb-TGFbRII, a-HVEM-TGFbRII). In an additional aspect, the ALT may comprise the antibody wherein the heavy chain is fused to TGFbRII ECD, and the light chain is fused to another receptor ECD that binds a ligand expressed on tumor cells or the TME (e.g. SIRPa ECD; BTLA ECD, TIM3 ECD, or PD1 ECD or SIGLEC10 ECD). In another aspect the ECD-ECD comprises TGFbRII ECD and another receptor ECD that binds a ligand expressed on tumor cells (e.g. PDL1/2, HVEM, CEACAM1/5, SIRPa). Examples of such ECD-ECDs include (TGFbRII ECD-Fc-BTLA ECD, TGFbRII ECD-Fc-PD1 ECD, TGFbRII ECD-Fc-TIM3 ECD, TGFbRII ECD-Fc-SIRPa ECD, TGFbRII ECD-Fc-SIGLEC10 ECD).

In another embodiment, the TGFb/TGFBR binding ALT or ECD-ECD binds TGFb and a T cell co-stimulatory molecule. In one aspect a the ALT comprises an antibody that binds a T cell co-stimulatory molecule (e.g. 41BB, OX40, CD40, ICOS, GITR, DNAM) is fused to TGFbRII ECD (e.g. a-41BB-TGFbRII ECD, a-OX40-TGFbRII ECD, a-CD40-TGFbRII ECD, a-ICOS-TGFbRII ECD, a-GITR-TGFbRII ECD, a-DNAM-TGFbRII ECD). In another aspect, the ALT may be any antibody wherein the heavy chain is fused to TGFbRII ECD, and the light chain is fused to another receptor ECD that binds a ligand expressed on tumor cells or the TME (e.g. SIRPa ECD; BTLA ECD, TIM3 ECD, or PD1 ECD, SIGLEC10 ECD). In another aspect the ECD-ECD comprises TGFbRII ECD and another co-stimulatory ligand (e.g. 41BBL, OX40L, GITRL, CD40L, ICOSL). Examples of such ECD-ECDs include (TGFbRII ECD-Fc-41BBL, TGFbRII ECD-Fc-OX40L, TGFbRII ECD-Fc-ICOSL).

In another embodiment, the TGFb/TGFbR binding ALT or ECD-ECD comprises an antibody that binds either CD73 or CD39, fused to TGFbRII ECD (a-CD73-TGFbRII and a-CD39-TGFbRII)

In another aspect, the ALT may be any antibody wherein the heavy chain is fused to TGFbRIIR ECD, and the light chain is fused to another receptor ECD that binds a ligand expressed on tumor cells or the TME (e.g. SIRPa ECD; BTLA ECD, TIM3 ECD, or PD1 ECD, SIGLEC10 ECD).

In one embodiment, an ALT comprises an antibody that binds a TH17-associated cytokine or cytokine receptor fused to a receptor ECD that binds a ligand expressed on tumor cells or the TME. In various examples the additional receptor ECD fused to the heavy or light chain is TGFbRII ECD, VEGFR ECD, PD1 ECD, TIM3 ECD, BTLA ECD, SIRPa ECD, or SIGLEC10 ECD. In one aspect a first receptor ECD is fused to the heavy chain and a second receptor ECD is fused to the light chain. In another aspect, the ALT may be an antibody wherein the heavy chain is fused to either TGFbRII R ECD or VEGFR ECD, and the light chain is fused to another receptor ECD (e.g. SIRPa ECD; BTLA ECD, TIM3 ECD, or PD1 ECD, SIGLEC10 ECD).

In some embodiments, fusion proteins of the invention disrupt cytokines/cytokine receptor interactions involved in TH17 cell differentiation and function: IL23/IL23R, IL1/IL1R, IL6/IL6R; IL17/IL17R.

In one embodiment the ALT comprises an antibody that binds IL17R, fused to one or more of the following: TGFbRII ECD, VEGFR ECD, PD1 ECD, TIM3 ECD, BTLA ECD, SIRPa ECD, or SIGLEC10 ECD. Exemplary molecules include anti-IL17R-TGFbRII ECD, anti-IL17R-VEGFR ECD, anti-IL17R-PD1 ECD, anti-IL17R-TIM3 ECD, anti-IL17R-BTLA ECD, anti-IL17R-SIRPa ECD, or anti-IL17R-SIGLEC10 ECD. In one aspect a first receptor ECD is fused to the heavy chain and a second receptor ECD is fused to the light chain. In one aspect, the heavy chain is fused to either TGFbRII or VEGFR ECD, and the light chain is fused to an additional receptor ECD selected from the following PD1 ECD, TIM3 ECD, BTLA ECD, SIRPa ECD, or SIGLEC10 ECD. Examples of these ALTs include anti-IL17R-TGFbRII ECD-BTLA ECD, anti-IL17R-TGFbRII ECD-PD1 ECD, anti-IL17R-VEGFR ECD-PD1 ECD, anti-IL17R-VEGFR ECD-BTLA ECD, anti-IL17R-BTLA ECD-PD1 ECD, anti-IL17R-BTLA ECD-SIRPa ECD, anti-IL17R-BTLA ECD-TIM3 ECD, anti-IL17R-SIRPa ECD-PD1 ECD.

In one embodiment the ALT comprises an antibody that binds IL23 or IL23R, fused to one or more of the following: TGFbRII ECD, VEGFR ECD, PD1 ECD, TIM3 ECD, BTLA ECD, SIRPa ECD, or SIGLEC10 ECD. Exemplary molecules include: anti-IL23-TGFbRII ECD, anti-1L23-VEGFR ECD, anti-IL23-PD1 ECD, anti-IL23-TIM3 ECD, anti-IIL23-BTLA ECD, anti-IIL23-SIRPa ECD, or anti-IL23-SIGLEC10 ECD. In one aspect, the heavy chain is fused to either TGFbRII or VEGFR ECD, and the light chain is fused to an additional receptor ECD selected from the following PD1 ECD, TIM3 ECD, BTLA ECD, SIRPa ECD, or SIGLEC10 ECD. Examples of these ALTs include anti-IL23R-TGFbRII ECD-BTLA ECD, anti-IL23R-TGFbRII ECD-BTLA ECD, anti-IL23R/IL23-TGFbRII ECD-PD1 ECD, anti-IL23R/IL23-VEGFR ECD-BTLA ECD. anti-IL23R/IL23-VEGFR ECD-BTLA ECD, anti-IL123R/IL23-BTLA ECD-PD1 ECD.

In one embodiment the ALT comprises an antibody that binds IL6R or IL6, fused to one or more of the following: TGFbRII ECD, VEGFR ECD, PD1 ECD, TIM3 ECD, BTLA ECD, SIRPa ECD, or SIGLEC10 ECD. In one embodiment the ALT comprises an antibody that binds IL1R or IL1, fused to one or more of the following: TGFbRII ECD, VEGFR ECD, PD1 ECD, TIM3 ECD, BTLA ECD, SIRPa ECD, or SIGLEC10 ECD. In one aspect, the heavy chain of the antibody is fused to either TGFbRII or VEGFR ECD, and the light chain is fused to an additional receptor ECD selected from the following PD1 ECD, TIM3 ECD, BTLA ECD, SIRPa ECD, or SIGLEC10 ECD.

In one embodiment, the present invention provides method of treating a subject having a disease or disorder comprising administering to the subject a fusion protein of the invention. In certain embodiments the patient has cancer.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The term “cancer” refers to a group diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

In some aspects administration can be in combination with one or more additional therapeutic agents. The phrases “combination therapy”, “combined with” and the like refer to the use of more than one medication or treatment simultaneously to increase the response. The fusion proteins of the present invention might for example be used in combination with other drugs or treatment in use to treat cancer. Specifically the administration of the composition of the present invention to a subject can be in combination with any anti-cancer therapies. Such therapies can be administered prior to, simultaneously with, sequentially with or following administration of the fusion protein of the present invention.

The term “anti-cancer therapy” or “anti-cancer treatment” as used herein is meant to refer to any treatment that can be used to treat cancer, such as surgery, radiotherapy, chemotherapy, immunotherapy, and checkpoint inhibitor therapy.

Examples of chemotherapy include treatment with a chemotherapeutic, cytotoxic or antineoplastic agents including, but not limited to, (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

Examples of immunotherapy include treatment with antibodies including, but not limited to, alemtuzumab, Avastin (bevacizumab), Bexxar (tositumomab), CDP 870, and CEA-Scan (arcitumomab), denosumab, Erbitux (cetuximab), Herceptin (trastuzumab), Humira (adalimumab), IMC-IIF 8, LeukoScan (sulesomab), MabCampath (alemtuzumab), MabThera (Rituximab), matuzumab, Mylotarg (gemtuzumab oxogamicin), natalizumab, NeutroSpec (Technetium (99mTc) fanolesomab), panitumamab, Panorex (Edrecolomab), ProstaScint (Indium-Ill labeled Capromab Pendetide), Raptiva (efalizumab), Remicade (infliximab), ReoPro (abciximab), rituximab, Simulect (basiliximab), Synagis (palivizumab), TheraCIM hR3, tocilizumab, Tysabri (natalizumab), Verluma (nofetumomab), Xolair (omalizumab), Zenapax (dacliximab), Zevalin (ibritumomab tiuxetan (IDEC-Y2B8) conjugated to yttrium 90), Gilotrif (afatinib), Lynparza (olaparib), Perj eta (pertuzumab), Otdivo (nivolumab), Bosulif (bosutinib), Cabometyx (cabozantinib), trastuzumab-dkst (Ogivri), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib).

“Checkpoint inhibitor therapy” is a form of cancer treatment that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), MR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

In some embodiments the fusion proteins may be used in in combination with another therapy including surgery, chemotherapy, radiation therapy, targeted small molecules, anti-angiogenic therapy or immunotherapy. In certain embodiments the fusion protein is administered with another fusion protein. Immunotherapy may include any immuno-oncologic drug selected from a broad range of agents, including antibodies, vaccines, adjuvant therapies, cytokines, oncolytic viruses, bispecific molecules, and cellular therapies. In a specific embodiment, the molecules of the invention may be administered to a subject in combination with (Chimeric Antigen Receptor (CAR) T cell therapy.

In one embodiment, the present invention provides for compositions comprising the previously described molecule or fusion protein and a pharmaceutical carrier.

In an additional embodiment, the present invention provides a method of treating a subject having cancer comprising administering to the subject the previously described molecules, fusion proteins or compositions. In one aspect, the molecule, fusion protein or composition is administered by intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal or ocular administrations, by infusion, inhalation, or nebulization or by parenteral administration.

The molecules of the invention may be used in conjunction or in combination with any other type of therapy including surgery, chemotherapy, radiation therapy, targeted small molecules, anti-angiogenic therapy or immunotherapy. Immunotherapy may include any immuno-oncologic drug selected from a broad range of agents, including antibodies, fusion proteins, vaccines, adjuvant therapies, cytokines, oncolytic viruses, bispecific molecules, and cellular therapies. In a specific embodiment, the molecules of the invention may be administered to a subject in combination with (Chimeric Antigen Receptor (CAR) T cell therapy. In various aspects, the molecules of the invention may be administered prior to, concurrently, sequentially, and/or following any other type of said therapy. In various aspects, the molecules of the invention may be administered in a composition with any other therapeutic agent.

In some embodiments, fusion proteins described in the invention and combination therapies counteract immune dysfunction in the tumor microenvironment. In some embodiments, fusion proteins described in the invention and combination therapies counteract angiogenesis in the tumor microenvironment.

In various embodiments, a subject may be administered one or more fusion proteins described in the invention. In some embodiments, the fusion proteins comprise one or more of TGFbRII ECD, VEGFR ECD, PD1 ECD, BTLA ECD, SIRPa ECD, TIM3 ECD, and SIGLEC10 ECD.

In various embodiments, a subject may be administered one or more fusion proteins described in the invention in combination with a polypeptide that disables an immune cell inhibitory molecule or T cell co-inhibitory molecule (e.g., CTLA-4, BTLA, TIM-3, CEACAM1, or CEACAM-5, TIGIT, PVRIG, VISTA, VSIG8, LAG-3, CD47, SIRPa, CD24, SIGLEC10, or LILRB1). In some embodiments, this polypeptide is an antibody. In other embodiments, the polypeptide is a fusion protein comprising the ECD of a T cell co-inhibitory molecule. In some embodiments, this polypeptide may be PD1-Fc, TIM3-Fc, or BTLA-Fc. In some embodiments, the polypeptide may be an anti-PD1/anti-PDL1 mAb. Exemplary such antibodies are anti-PD1 (e.g., nivolumab, pembrolizumab) and anti-PDL1 (e.g., durvalumab, avelumab, atezolizumab).

In various embodiments, a subject may be administered one or more fusion proteins described in the invention in combination with a second fusion protein described in the invention. In various aspects, this second fusion protein disables a T cell co-inhibitory molecule. In some embodiments, this second fusion protein comprises BTLA ECD, TIM-3 ECD, or PD-1 ECD.

In various embodiments, a subject may be administered one or more fusion proteins described in the invention in combination with an antibody or fusion protein that activates an T cell co-stimulatory molecule (e.g., OX40, 41BB, ICOS, GITR, HVEM, CD27, CD40, CD30, DNAM). In some embodiments, the fusion protein comprises the ECD of a T cell co-stimulatory ligand (e.g., OX40L, 41BBL, ICOSL, GITRL, LIGHT, CD70, CD40L, CD30L) that binds a T cell co-stimulatory receptor as an agonist. In one aspect, the T cell co-stimulatory ligand may be fused to a tumor-targeted antibody (e.g., anti-EGFR-LIGHT).

In various embodiments, a subject may be administered one or more fusion proteins described in the invention in combination with an inhibitor of TGFb/TGFbR. In some aspects, this TGFb/TGFbR inhibitor is selected from: a-TGFb antibody; a-TGFbR antibody; TGFbRII ECD containing fusion protein (e.g. TGFbRIIecd-Fc); TGFbR TKI (e.g. galunisertib); anti-GARP antibody; anti-LAP antibody; ALT comprising TGFbRII ECD; ALT that inhibits TGFb/TGFbR; fusion proteins described in this invention that inhibit TGFb/TGFbR.

In various embodiments, a subject may be administered one or more fusion proteins described in the invention in combination with an inhibitor of VEGF/VEGFR. In some aspects, the VEGF/VEGFR inhibitor may be selected from: anti-VEGF antibody (e.g., bevacizumab), anti-VEGFR antibody (e.g. ramucirumab), VEGFR kinase inhibitor (e.g., sunitinib, sorafenib, axitinib, cabozantinib, regorafenib, pazopanib, vandetanib, lenvatenib), or VEGFR ECD-Fc fusion protein (e.g., aflibercept).

In various embodiments, a subject may be administered one or more fusion proteins described in the invention in combination with an inhibitor of TH17 differentiation and/or function. In some aspects, this agent is an inhibitor of the interaction between IL23/IL23R, IL1/IL1R, IL6/IL6R, or IL17/IL17R. In some aspects, this agent is selected from: anti-IL17R antibody, anti-IL17 antibody, anti-IL17 nanobody; anti-IL6R antibody, anti-IL6 antibody; IL23 antibody, anti-IL23R antibody; anti-IL1R antibody, anti-IL1 antibody. In other aspects, this agent is a fusion protein described in the invention that inhibits IL23/IL23R, IL1/IL1R, IL6/IL6R, IL17/IL17R.

In various embodiments, a subject may be administered one or more fusion proteins described in the invention in combination with an immunocytokine or cytokine fusion protein comprising an active ligand or ligand fragment of IL12 or IL15.

In various embodiments, a subject may be administered one or more fusion proteins described in the invention in combination with a polypeptide that binds a tumor cell- or tumor antigen, tumor growth factor or growth factor receptor. In one aspect, this polypeptide is an antibody. In another aspect, this polypeptide is conjugated to a cytotoxic compound. In a further aspect, this polypeptide is an ADC. In a further aspect, this polypeptide is an ALT-DC.

In various embodiments, a subject may be administered one or more fusion proteins described in the invention in combination with a chimeric antigen receptor T cell (CAR T cell)

In various embodiments, a subject may be administered one or more fusion proteins described in the invention in combination with an immunotherapeutic agent.

In various embodiments, a subject may be administered one or more fusion proteins described in the invention in combination with an inhibitor of tumor cell signaling that promotes tumor cell survival, proliferation, invasion, and/or metastases; tumor angiogenesis; or immune dysfunction in the TME.

In various embodiments, a subject may be administered one or more fusion proteins described in the invention in combination with a chemotherapeutic or cytotoxic agent, a DNA repair inhibitor or PARP inhibitor, a tumor vaccine or viriolytic agent; or ionizing radiation.

Any VEGF or VEGFR-binding fusion protein of the invention may be used for the treatment of cancer. Any TGFb/TGFbR-inhibiting fusion protein of the invention may be used for the treatment of cancer. Any fusion protein of the invention that interrupts a cytokine/cytokine receptor interaction involved in TH17 cell differentiation or function may be used for the treatment of cancer. In some embodiments, this cytokine/cytokine receptor interaction involved in TH17 inhibition is selected from IL23/IL23R, IL1/IL1R, IL6/IL6R, or IL17/IL17R.

In one embodiment, a VEGF or VEGFR-binding fusion protein of the invention may be used in combination with a TGF/TGFbR-inhibiting fusion protein of the invention for the treatment of cancer.

In another embodiment, a VEGF or VEGFR-binding fusion protein of the invention may be used in combination with a TGFb/TGFbR inhibitor for the treatment of cancer. In some aspects, this TGFb/TGFbR inhibitor is selected from: a-TGFb antibody; a-TGFbR antibody; TGFbRII ECD containing fusion protein (e.g. TGFbRIIecd-Fc); TGFbR TKI (e.g. galunisertib); anti-GARP antibody; anti-LAP antibody; ALT comprising TGFbRII ECD.

In another embodiment, a TGFb/TGFbR-inhibiting fusion protein of the invention may be used in combination with a VEGF/VEGFR inhibitor for the treatment of cancer. In some aspects, the VEGF/VEGFR inhibitor may be selected from: anti-VEGF antibody (e.g., bevacizumab), anti-VEGFR antibody (e.g. ramucirumab), VEGFR kinase inhibitor (e.g., sunitinib, sorafenib, axitinib, cabozantinib, regorafenib, pazopanib, vandetanib, lenvatenib), or VEGFR ECD-Fc fusion protein (e.g., aflibercept).

In another embodiment, a VEGF/VEGFR-inhibiting fusion protein of the invention may be used in combination with a fusion protein of the invention that counteracts TH17 differentiation or function for the treatment of cancer.

In another embodiment, a VEGF or VEGFR-binding fusion protein of the invention may be used in combination with an agent that counteracts TH17 differentiation or function for the treatment of cancer. In some aspects, this agent is an inhibitor of the interaction between IL23/IL23R, IL1/IL1R, IL6/IL6R, or IL17/IL17R. In some aspects, this agent is selected from: anti-IL17R antibody, anti-IL17 antibody, anti-IL17 nanobody; anti-IL6R antibody, anti-IL6 antibody; IL23 antibody, anti-IL23R antibody; anti-IL1R antibody, anti-IL1 antibody.

In another embodiment, a TGFb/TGFbR-inhibiting fusion protein of the invention may be used in combination with a fusion protein of the invention that counteracts TH17 differentiation or function for the treatment of cancer.

In another embodiment, a TGFb/TGFbR-inhibiting fusion protein of the invention may be used in combination with an agent that counteracts TH17 differentiation or function for the treatment of cancer. In some aspects, this agent is an inhibitor of the interaction between IL23/IL23R, IL1/IL1R, IL6/IL6R, or IL17/IL17R. In some aspects, this agent is selected from: anti-IL17R antibody, anti-IL17 antibody, anti-IL17 nanobody; anti-IL6R antibody, anti-IL6 antibody; IL23 antibody, anti-IL23R antibody; anti-IL1R antibody, anti-IL1 antibody.

In another embodiment, a fusion protein of the invention that counteracts TH17 differentiation or function may be used in combination with a TGFb/TGFbR inhibitor for the treatment of cancer. In some aspects, this TGFb/TGFbR inhibitor is selected from: a-TGFb antibody; a-TGFbR antibody; TGFbRII ECD containing fusion protein (e.g. TGFbRIIecd-Fc); TGFbR TKI (e.g. galunisertib); anti-GARP antibody; anti-LAP antibody; ALT comprising TGFbRII ECD.

In another embodiment, a fusion protein of the invention that counteracts TH17 differentiation or function may be used in combination with a VEGF/VEGFR inhibitor for the treatment of cancer. In some aspects, the VEGF/VEGFR inhibitor may be selected from: anti-VEGF antibody (e.g., bevacizumab), anti-VEGFR antibody (e.g. ramucirumab), VEGFR kinase inhibitor (e.g., sunitinib, sorafenib, axitinib, cabozantinib, regorafenib, pazopanib, vandetanib, lenvatenib), or VEGFR ECD-Fc fusion protein (e.g., aflibercept).

In another embodiment, a TGFb/TGFbR-inhibiting fusion protein of the invention may be used in combination with an agent that inhibits the interaction of CD47 and SIRPa. In some aspects, the CD47/SIRPa inhibitor may be selected from: a-CD47 mAb (e.g., magrolimab), a-SIRPa mAb, SIRPa containing fusion protein (e.g., SIRPa-Fc). In another aspect, the agent that inhibits the interaction of CD47 and SIRPa is a fusion protein of the invention.

In another embodiment, a VEGF/VEGFR-binding fusion protein of the invention may be used in combination with an agent that inhibits the interaction of CD47 and SIRPa. In some aspects, the CD47/SIRPa inhibitor may be selected from: a-CD47 mAb (e.g., magrolimab), a-SIRPa mAb, or SIRPa containing fusion protein (e.g., SIRPa-Fc). In another aspect, the agent that inhibits the interaction of CD47 and SIRPa is a fusion protein of the invention.

In another embodiment, a fusion protein of the invention that counteracts TH17 differentiation or function may be used in combination with an agent that inhibits the interaction of CD47 and SIRPa. In some aspects, the CD47/SIRPa inhibitor may be selected from: a-CD47 mAb (e.g., magrolimab), a-SIRPa mAb, or SIRPa containing fusion protein (e.g., SIRPa-Fc). In another aspect, the agent that inhibits the interaction of CD47 and SIRPa is a fusion protein of the invention.

In another embodiment, a CD47/SIRPa-binding fusion protein of the invention may be used in combination with a TGFb/TGFbR inhibitor for the treatment of cancer. In some aspects, this TGFb/TGFbR inhibitor is selected from: a-TGFb antibody; a-TGFbR antibody; TGFbRII ECD containing fusion protein (e.g. TGFbRIIecd-Fc); TGFbR TKI (e.g. galunisertib); anti-GARP antibody; anti-LAP antibody; ALT comprising TGFbRII ECD. In one embodiment, a-CD73-TGFbRII is combined with anti-CD47 or SIRPa-Fc for the treatment of cancer. In one aspect, the anti-CD47 polypeptide is magrolimab.

In another embodiment, a CD47/SIRPa-binding fusion protein of the invention may be used in combination with a VEGF/VEGFR inhibitor for the treatment of cancer. In some aspects, the VEGF/VEGFR inhibitor may be selected from: anti-VEGF antibody (e.g., bevacizumab), anti-VEGFR antibody (e.g. ramucirumab), VEGFR kinase inhibitor (e.g., sunitinib, sorafenib, axitinib, cabozantinib, regorafenib, pazopanib, vandetanib, lenvatenib), or VEGFR ECD-Fc fusion protein (e.g., aflibercept).

In another embodiment, a CD47/SIRPa-binding fusion protein of the invention may be used in combination with an agent that counteracts TH17 differentiation or function for the treatment of cancer. In some aspects, this agent is an inhibitor of the interaction between IL23/IL23R, IL1/IL1R, IL6/IL6R, or IL17/IL17R. In some aspects, this agent is selected from: anti-IL17R antibody, anti-IL17 antibody, anti-IL17 nanobody; anti-IL6R antibody, anti-IL6 antibody; IL23 antibody, anti-IL23R antibody; anti-IL1R antibody, anti-IL1 antibody.

In one embodiment, a polypeptide that simultaneously binds VEGF and a component of the tumor microenvironment is combined with a polypeptide that simultaneously binds TGFb and a component of the tumor microenvironment. In various aspects, the component of the tumor microenvironment may be a tumor cell surface molecule. In another aspect, the component of the tumor microenvironment may be a immune cell surface molecule associated with tumor-infilitrating T cells such as TH17 cells (e.g., IL-23R) or Tregs (e.g., CTLA-4).

In some embodiments, a fusion protein described in the invention that inhibits TGFb/TGFbR is used for treatment of cancer in combination with a fusion protein described in the invention that inhibits one or more of: PD1/PDL1, CD47/SIRPa, BTLA/HVEM, TIM3/CEACAM, VEGF/VEGFR, SIGLEC10/CD24. In some embodiments, a fusion protein described in the invention that inhibits PD1/PDL1 is used for treatment of cancer in combination with a fusion protein described in the invention that inhibits one or more of: TGFb/TGFbR, CD47/SIRPa, BTLA/HVEM, TIM3/CEACAM, VEGF/VEGFR, SIGLEC10/CD24. In some embodiments, a fusion protein described in the invention that inhibits CD47/SIRPa is used for treatment of cancer in combination with a fusion protein described in the invention that inhibits one or more of: TGFb/TGFbR, PD1/PDL1, BTLA/HVEM, TIM3/CEACAM, VEGF/VEGFR, SIGLEC10/CD24. In some embodiments, a fusion protein described in the invention that inhibits BTLA/HVEM is used for treatment of cancer in combination with a fusion protein described in the invention that inhibits one or more of: TGFb/TGFbR, CD47/SIRPa, PD1/PDL1, TIM3/CEACAM, VEGF/VEGFR, SIGLEC10/CD24. In some embodiments, a fusion protein described in the invention that inhibits TIM3/CEACAM is used for treatment of cancer in combination with a fusion protein described in the invention that inhibits one or more of: TGFb/TGFbR, CD47/SIRPa, BTLA/HVEM, PD1/PDL1, VEGF/VEGFR, SIGLEC10/CD24. In some embodiments, a fusion protein described in the invention that inhibits VEGF/VEGFR is used for treatment of cancer in combination with a fusion protein described in the invention that inhibits one or more of: TGFb/TGFbR, CD47/SIRPa, BTLA/HVEM, TIM3/CEACAM, PD1/PDL1, SIGLEC10/CD24. In some embodiments, a fusion protein described in the invention that inhibits SIGLEC10/CD24 is used for treatment of cancer in combination with a fusion protein described in the invention that inhibits one or more of: TGFb/TGFbR, CD47/SIRPa, BTLA/HVEM, TIM3/CEACAM, VEGF/VEGFR, PD1/PDL1.

Sequences of the components and fusion proteins of the invention are in Table 1

TABLE 1

| | Sequence |
|---|---|
| SEQ ID NO: 1 | QVQLQQWGAGLLKPSETLSLTCAVYGGSFSGYYWSWIRQSPEKGLEWIGEI |
| anti-41BB | NHGGYVTYNPSLESRVTISVDTSKNQFSLKLSSVTAADTAVYYCARDYGPG |
| antibody | NYDWYFDLWGRGTLVTVSSASTKGPSVFPLAPCSRSTSESTAALGCLVKD |
| (urelumab) - | YFPEPVTVSWNSGALTSGVHTFPAVLQSSGLYSLSSVVTVPSSSLGTKTYTC |
| heavy chain | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGK |
| SEQ ID NO: 2 | EIVLTQSPATLSLSPGERATLSCRASQSVSSYLAWYQQKPGQAPRLLIYDAS |
| anti-41BB | NRATGIPARFSGSGSGTDFTLTISSLEPEDFAVYYCQQRSNWPPALTFCGGT |
| antibody | KVEIKRTVAAPSVFIFPPSDEQLKSGTASVVCLLNNFYPREAKVQWKVDNA |
| (urelumab) - light | LQSGNSQESVTEQDSKDSTYSLSSTLTLSKADYEKHKVYACEVTHQGLSSP |
| chain | VTKSFNRGEC |
| SEQ ID NO: 3 | EVQLVESGGGLVQPGGSLRLSCAASGYSITNDYAWNWVRQAPGKGLEWV |
| anti-CA125 | GYISYSGYTTYNPSLKSRFTISRDTSKNTLYLQMNSLRAEDTAVYYCARWT |
| antibody (ab1) - | SGLDYWGQGTLVTVSSCSTKGPSVFPLAPSSKSTSGGTAALGCLVKDYFPE |
| heavy chain | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 4 | DIQMTQSPSSLSASVGDRVTITCKASDLIHNWLAWYQQKPGKAFKLLIYGA |
| anti-CA125 | TSLETGVPSRFSGSGSGTDFTLTISSLQPEDFATYYCQQYWTTPFTFGQGTK |
| antibody (ab1) - | VEIKRTVAAPSVFIFPPSDEQLKSGTASVVCLLENFYPREAKVQWKVDNAL |
| light chain | QSGNSQESVTEQDSKDSTYSLSSTLTLSKADYEKHKVYACEVTHQGLSSFV |
| | TKSFERGEC |
| SEQ ID NO: 5 | EVQLVESGGGLVQPGGSLRLSCAASGYSITNDYAWNWVRQAPGKGLEWV |
| anti-CA125 | GYISYSGYTTYNPSLKSRFTISRDTSKNTLYLQMNSLRAEDTAVYYCARWT |
| antibody | SGLDYWGQGTLVTVSSASTKGPSVFPLAPSSKSTSGGTAALGCLVKDYFPE |
| (sofituzumab) - | PVTVSWNSGALTSGVHTFPAVLQSSGLYSLSSVVTVPSSSLGTQTYICNVNH |
| heavy chain | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 6 | DIQMTQSPSSLSASVGDRVTITCKASDLIHNWLAWYQQKPGKAPKLLIYGA |
| anti-CA125 | TSLETGVPSRFSGSGSGTDFTLTISSLQPEDFATYYCQQYWTTPFTFGQGTK |
| antibody | VEIKRTVAAPSVFIFPPSDEQLKSGTASVVCLLNNFYPREAKVQWKVDNAL |
| (sofituzumab) - | QSGNSQESVTEQDSKDSTYSLSSTLTLSKADYEKHKVYACEVTHQGLSSPV |
| light chain | TKSFNRGEC |
| SEQ ID NO: 7 | QVQLVESGGGSVQPGRSLRLSCEASGFTFEAYAMHWVRQPPGKGLEWVSS |
| anti-CA19-9 | INWNSGRIAYADSVKGRFTISRDNARNSLYLQMNSLRLEDTAFYYCAKDIR |
| antibody (MVT- | RFSTGGAEFEYWGQGTLVTVSSASTKGPSVFPLAPSSKSTSGGTAALGCLV |
| 5873) - heavy | KDYFPEPVTVSWNSGALTSGVHTFPAVLQSSGLYSLSSVVTVPSSSLGTQTY |
| chain | ICNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 8 | QSVLTQPPSASGTPGQRVTISCSGSSSNIGSNFVYWYQQLPGTAPKLLIYRN |
| anti-CA19-9 | NQRPSGVPDRFSGSRSGTSASLAISGLRSEDEADYYCAAWDDSLGGHYVFG |
| antibody (MVT- | TGTKVTVLGQPKAAPSVTLFPPSSEELQANKATLVCLISDFYPGAVTVAWK |
| 5873) - light chain | ADSSPVKAGVETTTPSKQSNNKYAASSYLSLTPEQWKSHRSYSCQVTHEGS |
| | TVEKTVAPTECS |
| SEQ ID NO: 9 | QVQLQQPGAELVKPGASVKMSCKASGYTFTSYNMHWVKQTPGRGLEWIG |
| anti-CD20 | AIYPGNGDTSYNQKFKGKATLTADKSSSTAYMQLSSLTSEDSAVYYCARST |
| antibody | YYGGDWYFNVWGAGTTVTVSAASTKGPSVFPLAPSSKSTSGGTAALGCLV |
| (rituximab) - | KDYFPEPVTVSWNSGALTSGVHTFPAVLQSSGLYSLSSVVTVPSSSLGTQTY |
| heavy chain | ICNVNHKPSNTKVDKKAEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 10 | QIVLSQSPAILSASPGEKVTMTCRASSSVSYIHWFQQKPGSSPKPWIYATSNL |
| anti-CD20 | ASGVPVRFSGSGSGTSYSLTISRVEAEDAATYYCQQWTSNPPTFGGGTKLEI |
| antibody | KRTVAAPSVFIFPPSDEQLKSGTASVVCLLNNFYPREAKVQWKVDNALQSG |
| (rituximab) - light | NSQESVTEQDSKDSTYSLSSTLTLSKADYEKHKVYACEVTHQGLSSPVTKS |
| chain | FNRGEC |
| SEQ ID NO: 11 | QIQLQQSGPEVVKPGASVKISCKASGYTFTDYYITWVKQKPGQGLEWIGWI |
| anti-CD30 | YPGSGNTKYNEKFKGKATLTVDTSSSTAFMQLSSLTSEDTAVYFCANYGN |
| antibody | YWFAYWGQGTQVTVSAASTKGPSVFPLAPSSKSTSGGTAALGCLVKDYFP |
| (brentuximab) - | EPVTVSWNSGALTSGVHTFPAVLQSSGLYSLSSVVTVPSSSLGTQTYICNVN |
| heavy chain | HKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPG |
| SEQ ID NO: 12 | DIVLTQSPASLAVSLGQRATISCKASQSVDFDGDSYMNWYQQKPGQPPKV |
| anti-CD30 | LIYAASNLESGIPARFSGSGSGTDFTLNIHPVEEEDAATYYCQQSNEDPWTF |
| antibody | GGGTKLEIKRTVAAPSVFIFPPSDEQLKSGTASVVCLLNNFYPREAKVQWK |
| (brentuximab) - | VDNALQSGNSQESVTEQDSKDSTYSLSSTLTLSKADYEKHKVYACEVTHQ |
| light chain | GLSSPVTKSFNRGEC |
| SEQ ID NO: 13 | QVQLQQSGAELAKPGASVKMSCKASGYTFTSYRMHWVKQRPGQGLEWIG |
| anti-CD33 | YINPSTGYTEYNQKFKDKATLTADKSSSTAYMQLSSLTFEDSAVYYCARGG |
| antibody | GVFDYWGQGTTLTVSSASTKGPSVFPLAPSSKSTSGGTAALGCLVKDYFPE |
| (gemtuzumab) - | PVTVSWNSGALTSGVHTFPAVLQSSGLYSLSSVVTVPSSSLGTQTYICNVNH |
| heavy chain | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 14 | DIQMTQSPSTLSASVGDRVTITCRASQSINTWLAWYQQKPGKAPKLLMYK |
| anti-CD33 | ASSLESGVPSRFIGSGSGTEFTLTISSLQPDDFATYYCQQYNSDSKMFGQGT |
| antibody | KVEVKRTVAAPSVFIFPPSDEQLKSGTASVVCLLNNFYPREAKVQWKVDN |
| (gemtuzumab) - | ALQSGNSQESVTEQDSKDSTYSLSSTLTLSKADYEKHKVYACEVTHQGLSS |
| light chain | PVTKSFNRGEC |
| SEQ ID NO: 15 | EVQLLESGGGLVQPGGSLRLSCAVSGFTFNSFAMSWVRQAPGKGLEWVSA |
| anti-CD38 | ISGSGGGTYYADSVKGRFTISRDNSKNTLYLQMNSLRAEDTAVYFCAKDKI |
| antibody | LWFGEPVFDYWGQGTLVTVSSASTKGPSVFPLAPSSKSTSGGTAALGCLVK |
| (daratumumab) - | DYFPEPVTVSWNSGALTSGVHTFPAVLQSSGLYSLSSVVTVPSSSLGTQTYI |
| heavy chain | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 16 | EIVLTQSPATLSLSPGERATLSCRASQSVSSYLAWYQQKPGQAPRLLIYDAS |
| anti-CD38 | NRATGIPARFSGSGSGTDFTLTISSLEPEDFAVYYCQQRSNWPPTFGQGTKV |
| antibody | EIKRTVAAPSVFIFPPSDEQLKSGTASVVCLLNNFYPREAKVQWKVDNALQ |
| (daratumumab) - | SGNSQESVTEQDSKDSTYSLSSTLTLSKADYEKHKVYACEVTHQGLSSPVT |
| light chain | KSFNRGEC |
| SEQ ID NO: 17 | QVOLVOSGSELKKPGASVKVSCKASGYTFTHYGMNWVRQAPGQGLKWM |
| anti-CD39 | GWINTYTGEPTYADDFKGRFVFSLDTSVSTAYLQISSLKAEDTAVYYCARR |
| antibody | RYEGNYVFYYFDYWGQGTTVTVSSASTKGPSVFPLAPSSKSTSGGTAALGC |
| (IPH5201) - heavy | LVKDYFPEPVTVSWNSGALTSGVHTFPAVLQSSGLYSLSSVVTVPSSSLGTQ |
| chain | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| SEQ ID NO: 18 | DIQMTQSPSSLSASVGDRVTITCRASENIYSYFSWYOQKPGKAPKLLIYTAK |
| anti-CD39 | TLAEGVPSRFSGSGSGTDFTLTISSLOPEDFATYYCQHHYVTPYTFGGGTKV |
| antibody | EIKRTVAAPSVFIFPPSDEOLKSGTASVVCLLNNFYPREAKVOWKVDNAL0 |
| (IPH5201) - light | SGNSQESVTEQDSKDSTYSLSSTLTLSKADYEKHKVYACEVTHOGLSSPVT |
| chain | KSFNRGEC |
| SEQ ID NO: 19 | EVQLQESGPGLVKPSETLSLTCTVSGYSITSNYYWNWIRQPPGKGLEWMG |
| anti-CD40 | YIRYDGSNNYNPSLKNRVTISRDTSKNQFSLKLSSVTAADTAVYYCARLDY |
| antibody (ABBV- | WGQGTTVTVSSASTKGPSVFPLAPSSKSTSGGTAALGCLVKDYFPEPVTVS |
| 428) - heavy chain | WNSGALTSGVHTFPAVLQSSGLYSLSSVVTVPSSSLGTQTYICNVNHKPSNT |
| | KVDKKVEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKN |
| | QVSLTCLVKGFYPSDIAVEWESNGQPENNYKTTPPVLDSDGSFFLYSKLTV |
| | DKSRWQQGNVFSCSVMHEALHNHYTQKSLSLSPGK |
| SEQ ID NO: 20 | DAVMTQTPLSLSVTPGQPASISCRSSQSLENTNGNTFLNWYLQKPGQSPQL |
| anti-CD40 | LIYRVSNRFSGVPDRFSGSGSGTDFTLKISRVEAEDVGVYYCLQVTHVPFTF |
| antibody (ABBV- | GQGTKLEIKRTVAAPSVFIFPPSDEQLKSGTASVVCLLNNFYPREAKVQWK |
| 428) - light chain | VDNALQSGNSQESVTEQDSKDSTYSLSSTLTLSKADYEKHKVYACEVTHQ |
| | GLSSPVTKSFNRGEC |
| SEQ ID NO: 21 | QVQLQQPGAELVKPGASVMMSCKASGYTFTNYNMHWVKQTPGQGLEWI |
| anti-CD47 | GTIYPGNDDTSYNQKFKDKATLTADKSSSAAYMQLSSLTSEDSAVYYCAR |
| antibody (5F9) - | GGYRAMDYWGQTSVTVSSASTKGPSVFPLAPSSKSTSGGTAALGCLVKDY |
| heavy chain | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 22 | DVLMTQTPLSLPVSLGDQASISCRSSQSIVYSNGNTYLGWYLQKPGQSPKL |
| anti-CD47 | LIYKVSNRFSGVPDRFSGSGSGTDFTLKISRVEAEDLGVYHCFQGSHVPYTF |
| antibody (5F9) - | GGGTKVEIKRTVAAPSVFIFPPSDEQLKSGTASVVCLLNNFYPREAKVQWK |
| light chain | VDNALQSGNSQESVTEQDSKDSTYSLSSTLTLSKADYEKHKVYACEVTHQ |
| | GLSSPVTKSFNRGEC |
| SEQ ID NO: 23 | EVQLVQSGAEVKKPGESLKISCKASGYAFSSSWINWVRQMPGKGLEWMG |
| anti-CD73 | RIYPRAGDTNYAGKFKDQVTISADKSISTAYLQWSSLKASDTAMYYCASLL |
| antibody | DYSMDYWGQGTLVTVSSASTKGPSVFPLAPSSKSTSGGTAALGCLVKDYF |
| (GS1423) - heavy | PEPVTVSWNSGALTSGVHTFPAVLQSSGLYSLSSVVTVPSSSLGTQTYICNV |
| chain | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| SEQ ID NO: 24 | DIQMTQSPSSLSASVGDRVTITCRASQDISNYLNWYQQKPGKAPKLLIYYTS |
| anti-CD73 | RLHSGVPSRFSGSGSGTDFTFTISSLQPEDIATYYCQQGNTLPLTFGQGTKVE |
| antibody | IKRTVAAPSVFIFPPSDEQLKSGTASVVCLLNNFYPREAKVQWKVDNALQS |
| (GS1423) - light | GNSQESVTEQDSKDSTYSLSSTLTLSKADYEKHKVYACEVTHQGLSSPVTK |
| chain | SFNRGEC |
| SEQ ID NO: 25 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| antibody | PWFAYWGQGTPVTVSSASTKGPSVFPLAPSSKSTSGGTAALGCLVKDYFPE |
| (labetuzumab) - | PVTVSWNSGALTSGVHTFPAVLQSSGLYSLSSVVTVPSSSLGTQTYICNVNH |
| heavy chain | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| | DIQLTQSPSSLSASVGDRVTITCKASQDVGTSVAWYQQKPGKAPKLLIYWT |
| SEQ ID NO: 26 | STRHTGVPSRFSGSGSGTDFTFTISSLQPEDIATYYCQQYSLYRSFGQGTKVE |
| anti-CEACAM5 | IKRTVAAPSVFIFPPSDEQLKSGTASVVCLLNNFYPREAKVQWKVDNALQS |
| antibody | GNSQESVTEQDSKDSTYSLSSTLTLSKADYEKHKVYACEVTHQGLSSPVTK |
| (labetuzumab) - | SFNRGEC |
| light chain | |
| SEQ ID NO: 27 | QVQLVESGGGVVQPGRSLRLSCAASGFTFSSYTMHWVRQAPGKGLEWVT |
| anti-CTLA4 | FISYDGNNKYYADSVKGRFTISRDNSKNTLYLQMNSLRAEDTAIYYCARTG |
| antibody | WLGPFDYWGQGTLVTVSSASTKGPSVFPLAPSSKSTSGGTAALGCLVKDYF |
| (ipilimumab) - | PEPVTVSWNSGALTSGVHTFPAVLQSSGLYSLSSVVTVPSSSLGTQTYICNV |
| heavy chain | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQ |
| SEQ ID NO: 28 | EIVLTQSPGTLSLSPGERATLSCRASQSVGSSYLAWYQQKPGQAPRLLIYGA |
| anti-CTLA4 | FSRATGIPDRFSGSGSGTDFTLTISRLEPEDFAVYYCQQYGSSPWTFGQGTK |
| antibody | VEIKRTVAAPSVFIFPPSDEQLKSGTASVVCLLNNFYPREAKVQWKVDNAL |
| (ipilimumab) - | QSGNSQESVTEQDSKDSTYSLSSTLTLSKADYEKHKVYACEVTHQGLSSPV |
| light chain | TKSFNRGEC |
| SEQ ID NO: 29 | QVQLVQSGAEVKKPGASVKVSCKASGYTFTNYGMNWVRQAPGQGLEWM |
| anti-DLL3 | GWINTYTGEPTYADDFKGRVTMTTDTSTSTAYMELRSLRSDDTAVYYCAR |
| antibody | IGDSSPSDYWGQGTLVTVSSASTKGPSVFPLAPSSKSTSGGTAALGCLVKD |
| (rovalpituzumab) - | YFPEPVTVSWNSGALTSGVHTFPAVLQSSGLYSLSSVVTVPSSSLGTQTYIC |
| heavy chain | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPG |
| SEQ ID NO: 30 | EIVMTQSPATLSVSPGERATLSCKASQSVSNDVVWYQQKPGQAPRLLIYYA |
| anti-DLL3 | SNRYTGIPARFSGSGSGTEFTLTISSLQSEDFAVYYCQQDYTSPWTFGQGTK |
| antibody | LEIKRTVAAPSVFIFPPSDEQLKSGTASVVCLLNNFYPREAKVQWKVDNAL |
| (rovalpituzumab) - | QSGNSQESVTEQDSKDSTYSLSSTLTLSKADYEKHKVYACEVTHQGLSSPV |
| light chain | TKSFNRGEC |
| SEQ ID NO: 31 | EVQLVESGGGLVQPGGSLRLSCAASGYTFTNYGMNWVRQAPGKGLEWVG |
| anti-DLL4 | WINTYTGEPTYAADFKRRFTFSLDTSKSTAYLQMNSLRAEDTAVYYCAKY |
| antibody (ABT- | PHYYGSSHWYFDVWGQGTLVTVSSASTKGPSVFPLAPSSKSTSGGTAALG |
| 165) - heavy chain | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPEAAGGPSVFLFPPK |
| | PKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| SEQ ID NO: 32 | DIQMTQSPSSLSASVGDRVTITCSASQDISNYLNWYQQKPGKAPKVLIYFTS |
| anti-DLL4 | SLHSGVPSRFSGSGSGTDFTLTISSLQPEDFATYYCQQYSTVPWTFGQGTKV |
| antibody (ABT- | EIKRTVAAPSVFIFPPSDEQLKSGTASVVCLLNNFYPREAKVQWKVDNALQ |
| 165) - light chain | SGNSQESVTEQDSKDSTYSLSSTLTLSKADYEKHKVYACEVTHQGLSSPVT |
| | KSFNRGEC |
| SEQ ID NO: 33 | QVQLVQSGAEVKKPGSSVKVSCKASGGTFSSYWIEWVRQAPGQGLEWMG |
| anti -DPEP3 | EILPGSGNTYYNERFKDRVTITADESTSTAYMELSSLRSEDTAVYYCARRA |
| antibody (ab1) - | AAYYSNPEWFAYWGQGTLVTVSSASTKGPSVFPLAPSSKSTSGGTAALGCL |
| heavy chain | VKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPG |
| SEQ ID NO: 34 | EIVLTQSPATLSLSPGERATLSCTASSSVNSFYLHWYQQKPGLAPRLLIYSTS |
| anti-DPEP3 | NLASGIPDRFSGSGSGTDFTLTISRLEPEDFAVYYCHQYHRSPYTFGQGTKL |
| antibody (ab1) - | EIKRTVAAPSVFIFPPSDEQLKSGTASVVCLLNNFYPREAKVQWKVDNALQ |
| light chain | SGNSQESVTEQDSKDSTYSLSSTLTLSKADYEKHKVYACEVTHQGLSSPVT |
| | KSFNRGEC |
| SEQ ID NO: 35 | QVQLVQSGAEVKKPGSSVKVSCKASGGTFSSYWIEWVRQAPGQGLEWMG |
| anti-DPEP3 | EILPGSGNTYYNERFKDRVTITADESTSTAYMELSSLRSEDTAVYYCARRA |
| antibody (ab2) - | AAYYSNPEWFAYWGQGTLVTVSSASTKGPSVFPLAPSSKSTSGGTAALGCL |
| heavy chain | VKDYFPEPVTVSWNSGALTSGVHTFPAVLQSSGLYSLSSVVTVPSSSLGTQT |
| | YICNVNHKPSNTKVDKKVEPKSSDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPG |
| SEQ ID NO: 36 | QVQLQESGPGLVKPSETLSLTCTVSGGSVSSGDYYWTWIRQSPGKGLEWIG |
| anti-EGFR | HIYYSGNTNYNPSLKSRLTISIDTSKTQFSLKLSSVTAADTAIYYCVRDRVTG |
| antibody | AFDIWGQGTMVTVSSASTKGPSVFPLAPCSRSTSESTAALGCLVKDYFPEPV |
| (panitumumab) - | TVSWNSGALTSGVHTFPAVLQSSGLYSLSSVVTVPSSNFGTQTYTCNVDHK |
| heavy chain | PSNTKVDKTVERKCCVECPPCPAPPVAGPSVFLFPPKPKDTLMISRTPEVTC |
| | VVVDVSHEDPEVQFNWYVDGVEVHNAKTKPREEQFNSTFRVVSVLTVVH |
| | QDWLNGKEYKCKVSNKGLPAPIEKTISKTKGQPREPQVYTLPPSREEMTKN |
| | QVSLTCLVKGFYPSDIAVEWESNGQPENNYKTTPPMLDSDGSFFLYSKLTV |
| | DKSRWQQGNVFSCSVMHEALHNHYTQKSLSLSPGK |
| SEQ ID NO: 37 | DIQMTQSPSSLSASVGDRVTITCQASQDISNYLNWYQQKPGKAPKLLIYDA |
| anti-EGFR | SNLETGVPSRFSGSGSGTDFTFTISSLQPEDIATYFCQHFDHLPLAFGGGTKV |
| antibody | EIKRTVAAPSVFIFPPSDEQLKSGTASVVCLLNNFYPREAKVQWKVDNALQ |
| (panitumumab) - | SGNSQESVTEQDSKDSTYSLSSTLTLSKADYEKHKVYACEVTHQGLSSPVT |
| light chain | KSFNRGEC |
| SEQ ID NO: 38 | QVQLQESGPGLVKPSQTLSLTCTVSGGSISSGDYYWSWIRQPPGKGLEWIG |
| anti-EGFR | YIYYSGSTDYNPSLKSRVTMSVDTSKNQFSLKVNSVTAADTAVYYCARVSI |
| antibody | FGVGTFDYWGQGTLVTVSSASTKGPSVLPLAPSSKSTSGGTAALGCLVKDY |
| (necitumumab) - | FPEPVTVSWNSGALTSGVHTFPAVLQSSGLYSLSSVVTVPSSSLGTQTYICN |
| heavy chain | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 39 | EIVMTQSPATLSLSPGERATLSCRASQSVSSYLAWYQQKPGQAPRLLIYDAS |
| anti-EGFR | NRATGIPARFSGSGSGTDFTLTISSLEPEDFAVYYCHQYGSTPLTFGGGTKA |
| antibody | EIKRTVAAPSVFIFPPSDEQLKSGTASVVCLLNNFYPREAKVQWKVDNALQ |
| (necitumumab) - | SGNSQESVTEQDSKDSTYSLSSTLTLSKADYEKHKVYACEVTHQGLSSPVT |
| light chain | KSFNRGEC |
| SEQ ID NO: 40 | EVQLQESGPGLVKPSQTLSLTCTVSGYSISNDFAWNWIRQLPGKGLEWMG |
| anti-EGFR | YISYKGNTRYQPSLKSRITISRDTSKNQFFLKLNSVTAADTATYYCVTASRG |
| antibody (ABBV- | LPYWGQGTLVTVSSASTKGPSVFPLAPSSKSTSGGTAALGCLVKDYFPEPV |
| 321) - heavy chain | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ ID NO: 41 | DIQMTQSPSSMSVSVGDRVTITCHSSQDITYNIGWLQQKPGKSFKGLIYHGA |
| anti-EGFR | NLDDGVPSRFSGSGSGTDYTLTISSLQPEDFATYYCVQYDEFPWTFGGGTK |
| antibody (ABBV- | LEIKRTVAAPSVFIFPPSDEQLKSGTASVVCLLNNFYPREAKVQWKVDNAL |
| 321) - light chain | QSGNSQESVTEQDSKDSTYSLSSTLTLSKADYEKHKVYACEVTHQGLSSPV |
| | TKSFNRGEC |
| SEQ ID NO: 42 | QVQLKQSGPGLVQPSQSLSITCTVSGFSLTNYGVHWVRQSPGKGLEWLGVI |
| anti-EGFR | WSGGNTDYNTPFTSRLSINKDNSKSQVFFKMNSLQSNDTAIYYCARALTYY |
| antibody | DYEFAYWGQGTLVTVSAASTKGPSVFPLAPSSKSTSGGTAALGCLVKDYFP |
| (cetuximab) - | EPVTVSWNSGALTSGVHTFYAVLQSSGLYSLSSVVTVPSSSLGTQTYICNVN |
| heavy chain | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 43 | DILLTQSPVILSVSPGERVSFSCRASQSIGTNIHWYQQRTNGSPRLLIKYASES |
| anti-EGFR | ISGIPSRFSGSGSGTDFTLSINSVESEDIADYYCQQNNNWPTTFGAGTKLELK |
| antibody | RTVAAPSVFIFPPSDEQLKSGTASVVCLLNNFYPREAKVQWKVDNALQSGN |
| (cetuximab) - light | SQESVTEQDSKDSTYSLSSTLTLSKADYEKHKVYACEVTHQGLSSPVTKSF |
| chain | NRGEC |
| SEQ ID NO: 44 | DVQLQESGPGLVKPSQSLSLTCTVTAYSVTSDYAWNWIRQFPGNKLEWMG |
| anti-EGFRvIII | YISYSGTTRYNPSLKSRISITRDTSKNQFFLQLNSMTAEDTATYYCSRQGRG |
| antibody (ab1) - | FPYWGQGTLVTVSAASTKGPSVFPLAPSSKSTSGGTAALGCLVKDYFPEPV |
| heavy chain | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ ID NO: 45 | DILMTQSPSSMSVSLGDTVSITCHASQDINSNIGWLQQKPGKSFKGLIYHGT |
| anti-EGFRvIII | NLEDGVPSRFSGSGSGADYSLTISSLESEDFADYYCVQYAQFPWTFGGGTK |
| antibody (ab1) - | LEIKRTVAAPSVFIFPPSDEQLKSGTASVVCLLNNFYPREAKVQWKVDNAL |
| light chain | QSGNSQESVTEQDSKDSTYSLSSTLTLSKADYEKHKVYACEVTHQGLSSPV |
| | TKSFNRGEC |
| SEQ ID NO: 46 | QVQLQESGPGLVKPSQTLSLTCTVSGYSISSDFAWNWIRQPPGKGLEWMG |
| anti-EGFRvIII | YISYSGNTRYQPSLKSRITISRDTSKNQFFLKLNSVTAADTATYYCVTAGRG |
| antibody | FPYWGQGTLVTVSSASTKGPSVFPLAPSSKSTSGGTAALGCLVKDYFPEPV |
| (depatuxizumab) - | TVSWNSGALTSGVHTFPAVLQSSGLYSLSSVVTVPSSSLGTQTYICNVNHKP |
| heavy chain | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ ID NO: 47 | DIQMTQSPSSMSVSVGDRVTITCHSSQDINSNIGWLQQKPGKSFKGLIYHGT |
| anti-EGFRvIII | NLDDGVPSRFSGSGSGTDYTLTISSLQPEDFATYYCVQYAQFPWTFGGGTK |
| antibody | LEIKRTVAAPSVFIFPPSDEQLKSGTASVVCLLNNFYPREAKVQWKVDNAL |
| (depatuxizumab) - | QSGNSQESVTEQDSKDSTYSLSSTLTLSKADYEKHKVYACEVTHQGLSSPV |
| light chain | TKSFNRGEC |
| SEQ ID NO: 48 | QVQLVQPGAEVRKPGASVKVSCKASGYRFTSYYIDWVRQAPGQGLEWMG |
| anti-GARP | RIDPEDAGTKYAQKFQGRVTMTADTSTSTVYVELSSLRSEDTAVYYCARY |
| antibody (ARGX- | EWETVVVGDLMYEYEYWGQGTLVTVSSASTKGPSVFPLAPCSRSTSESTA |
| 115) - heavy chain | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLG |
| SEQ ID NO: 49 | DIQMTQSPSSLSASVGDRVTITCQASQSISSYLAWYQQKPGQAPKILIYGAS |
| anti-GARP | RLKTGVPSRFSGSGSGTSFTLTISSLEPEDAATYYCQQYASVPVTFGQGTKV |
| antibody (ARGX- | EIKRTVAAPSVFIFPPSDEQLKSGTASVVCLLNNFYPREAKVQWKVDNALQ |
| 115) - light chain | SGNSQESVTEQDSKDSTYSLSSTLTLSKADYEKHKVYACEVTHQGLSSPVT |
| | KSFNRGEC |
| SEQ ID NO: 50 | EVQLLQSGPELEKPGASVMISCKASGSSFTGYNMNWVRQNIGKSLEWIGAI |
| anti-GD2 | DPYYGGTSYNQKFKGRATLTVDKSSSTAYMHLKSLTSEDSAVYYCVSGME |
| antibody | YWGQGTSVTVSSASTKGPSVFPLAPSSKSTSGGTAALGCLVKDYFPEPVTV |
| (dinutuximab) - | SWNSGALTSGVHTFPAVLQSSGLYSLSSVVTVPSSSLGTQTYICNVNHKPSN |
| heavy chain | TKVDKRVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVL |
| | HQDWLNGKEYKCKVSNKALPAPIEKTISKAKGQPREPQVYTLPPSREEMTK |
| | NQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| | EIVMTQSPATLSVSPGERATLSCRSSQSLVHRNGNTYLHWYLQKPGQSPKL |
| SEQ ID NO: 51 | LIHKVSNRFSGVPDRFSGSGSGTDFTLKISRVEAEDLGVYFCSQSTHVPPLTF |
| anti-GD2 | GAGTKLELKRTVAAPSVFIFPPSDEQLKSGTASVVCLLNNFYPREAKVQWK |
| antibody | VDNALQSGNSQESVTEQDSKDSTYSLSSTLTLSKADYEKHKVYACEVTHQ |
| (dinutuximab) - | GLSSPVTKSFNRGEC |
| light chain | |
| SEQ ID NO: 52 | EVQLVESGGGLVQPGGSLRLSCAASGFTFTDYTMDWVRQAPGKGLEWVA |
| anti-HER2 | DVNPNSGGSIYNQRFKGRFTLSVDRSKNTLYLQMNSLRAEDTAVYYCARN |
| antibody | LGPSFYFDYWGQGTLVTVSSASTKGPSVFPLAPSSKSTSGGTAALGCLVKD |
| (pertuzumab) - | YFPEPVTVSWNSGALTSGVHTFPAVLQSSGLYSLSSVVTVPSSSLGTQTYIC |
| heavy chain | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPG |
| SEQ ID NO: 53 | DIQMTQSPSSLSASVGDRVTITCKASQDVSIGVAWYQQKPGKAPKLLIYSAS |
| anti-HER2 | YRYTGVPSRFSGSGSGTDFTLTISSLQPEDFATYYCQQYYIYPYTFGQGTKV |
| antibody | EIKRTVAAPSVFIFPPSDEQLKSGTASVVCLLNNFYPREAKVQWKVDNALQ |
| (pertuzumab) - | SGNSQESVTEQDSKDSTYSLSSTLTLSKADYEKHKVYACEVTHQGLSSPVT |
| light chain | KSFNRGEC |
| SEQ ID NO: 54 | EVQLVESGGGLVQPGGSLRLSCAASGFNIKDTYIHWVRQAPGKGLEWVAR |
| anti-HER2 | IYPTNGYTRYADSVKGRFTISADTSKNTAYLQMNSLRAEDTAVYYCSRWG |
| antibody | GDGFYAMDYWGQGTLVTVSSASTKGPSVFPLAPSSKSTSGGTAALGCLVK |
| (trastuzumab) - | DYFPEPVTVSWNSGALTSGVHTFPAVLQSSGLYSLSSVVTVPSSSLGTQTYI |
| heavy chain | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 55 | DIQMTQSPSSLSASVGDRVTITCRASQDVNTAVAWYQQKPGKAPKLLIYSA |
| anti-HER2 | SFLYSGVPSRFSGSRSGTDFTLTISSLQPEDFATYYCQQHYTTPPTFGQGTKV |
| antibody | EIKRTVAAPSVFIFPPSDEQLKSGTASVVCLLNNFYPREAKVQWKVDNALQ |
| (trastuzumab) - | SGNSQESVTEQDSKDSTYSLSSTLTLSKADYEKHKVYACEVTHQGLSSPVT |
| light chain | KSFNRGEC |
| SEQ ID NO: 56 | QVQLQESGPGLVKPSETLSLTCTVSGGSISIYYWSWIRQPPGKGLEWIGYVY |
| anti-HGF | YSGSTNYNPSLKSRVTISVDTSKNQFSLKLNSVTAADTAVYYCARGGYDF |
| antibody | WSGYFDYWGQGTLVTVSSASTKGPSVFPLAPCSRSTSESTAALGCLVKDYF |
| (rilotumumab) - | PEPVTVSWNSGALTSGVHTFYAVLQSSGLYSLSSVVTVPSSNFGTQTYTCN |
| heavy chain | VDHKPSNTKVDKTVERKCCVECPPCPAPPVAGPSVFLFPPKPKDTLMISRTP |
| | EVTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGK |
| SEQ ID NO: 57 | EIVMTQSPATLSVSPGERATLSCRASQSVDSNLAWYRQKPGQAPRLLIYGA |
| anti-HGF | STRATGIPARFSGSGSGTEFTLTISSLQSEDFAVYYCQQYINWPPITFGQGTR |
| antibody | LEIKRTVAAPSVFIFPPSDEQLKSGTASVVCLLNNFYPREAKVQWKVDNAL |
| (rilotumumab) - | QSGNSQESVTEQDSKDSTYSLSSTLTLSKADYEKHKVYACEVTHQGLSSPV |
| light chain | TKSFNRGEC |
| SEQ ID NO: 58 | QVQLVQSGAEVKKPGSSVKVSCKASGYTFTDYAMHWVRQAPGQGLEWM |
| anti-ICOS | GLISTYSDHTNYNQKFQGRVTITADKSTSTAYMELSSLRSEDTAVYYCGRN |
| antibody | NYGNYGWYFDVWGQGTTVTVSS |
| (GSK3359609) - | |
| heavy chain | |
| SEQ ID NO: 59 | EIVLTQSPATLSLSPGERATLSCSASSSVSYMHWYQQKPGQAPRLLIYDTSK |
| anti-ICOS | LASGIPARFSGSGSGTDYTLTISSLEPEDFAVYYCFQGSGYPYTFGOGTKLEI |
| antibody | KRTVAAPSVFIFPPSDEQLKSGTASVVCLLNNFYPREAKVQWKVDNALQSG |
| (G5K3359609) - | NSQESVTEQDSKDSTYSLSSTLTLSKADYEKHKVYACEVTHQGLSSPVTKS |
| light chain | FNRGEC |
| SEQ ID NO: 60 | QVQLQQPGAELVRPGASVKLSCKASGYTFSNYLMNWVKQRPEQDLDWIG |
| anti-IL13Ra2 | RIDPYDGDIDYNQNFKDKAILTVDKSSSTAYMQLSSLTSEDSAVYYCARGY |
| antibody (ab1) - | GTAYGVDYWGQGTSVTVSSASTKGPSVFPLAPSSKSTSGGTAALGCLVKD |
| heavy chain | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| SEQ ID NO: 61 | DIVLTQSPASLAVSLGQRATISCRASESVDNYGISFMNWFQQKPGQPPKLLI |
| anti-IL13Ra2 | YAASRQGSGVPARFSGSGSGTDFSLNIHPMEEDDTAMYFCQQSKEVPWTF |
| antibody (ab1) - | GGGTKLEIKRTVAAPSVFIFPPSDEQLKSGTASVVCLLNNFYPREAKVQWK |
| light chain | VDNALQSGNSQESVTEQDSKDSTYSLSSTLTLSKADYEKHKVYACEVTHQ |
| | GLSSPVTKSFNRGEC |
| SEQ ID NO: 62 | QVQLVQSGAEVKKPGASVKVSCKASGYTFTRYGISWVRQAPGQGLEWMG |
| anti-IL17R | WISTYSGNTNYAQKLQGRVTMTTDTSTSTAYMELRSLRSDDTAVYYCARR |
| antibody | QLYFDYWGQGTLVTVSSASTKGPSVFPLAPCSRSTSESTAALGCLVKDYFP |
| (brodalumab) - | EPVTVSWNSGALTSGVHTFPAVLQSSGLYSLSSVVTVPSSNFGTQTYTCNV |
| heavy chain | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGK |
| SEQ ID NO: 63 | EIVMTQSPATLSVSPGERATLSCRASQSVSSNLAWFQQKPGQAPRPLIYDAS |
| anti-IL17R | TRATGVPARFSGSGSGTDFTLTISSLQSEDFAVYYCQQYDNWPLTFGGGTK |
| antibody | VEIKRTVAAPSVFIFPPSDEQLKSGTASVVCLLNNFYPREAKVQWKVDNAL |
| (brodalumab) - | QSGNSQESVTEQDSKDSTYSLSSTLTLSKADYEKHKVYACEVTHQGLSSPV |
| light chain | TKSFNRGEC |
| SEQ ID NO: 64 | EVQLVQSGAEVKKPGASVKVSCKASGYTFKYYGMNWVRQAPGQGLERM |
| anti-IL1a | GWINTYTGQSTYADDFKGRVTFTLDTSTSTAYMELSSLRSEDTAVYYCAR |
| antibody | DIYYYGSDFAMDYWGQGTTVTVSSASTKGPSVFPLAPSSKSTSGGTAALGC |
| (3D12r16) - heavy | LVKDYFPEPVTVSWNSGALTSGVHTFPAVLQSSGLYSLSSVVTVPSSSLGTQ |
| chain | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| SEQ ID NO: 65 | DIQMTQSPSSLSASVGDRVTITCRASQDISNMLNWYQQKPGKAPKLLIYYT |
| anti-IL1a | SRLKPGVPSRFSGSGSGTDYTFTISSLQPEDIATYFCQQGKTAPYTFGQGTKL |
| antibody | EIKRTVAAPSVFIFPPSDEQLKSGTASVVCLLNNFYPREAKVQWKVDNALQ |
| (3D12r16) - light | SGNSQESVTEQDSKDSTYSLSSTLTLSKADYEKHKVYACEVTHQGLSSPVT |
| chain | KSFNRGEC |
| SEQ ID NO: 66 | EVQLVESGGGVVQPGRSLRLSCSASGFIFSRYDMSWVRQAPGKGLEWVAY |
| anti-IL1b | ISHGGAGTYYPDSVKGRFTISRDNSKNTLFLQMDSLRAEDTAVYYCARGG |
| antibody (E26.35) | VYKGYFDVWGQGTPVTVSSASTKGPSVFPLAPSSKSTSGGTAALGCLVKD |
| - heavy chain | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| SEQ ID NO: 67 | DIQMTQSPSSLSASVGDRVTITCRASGNIHNYLTWYQQTPGKAPKLLIYNA |
| anti-IL1b | KTLADGVPSRFSGSGSGTDYTFTISSLQPEDIATYYCQHFWSIPYTFGQGTK |
| antibody (E26.35) | LQITRTVAAPSVFIFPPSDEQLKSGTASVVCLLNNFYPREAKVQWKVDNAL |
| - light chain | QSGNSQESVTEQDSKDSTYSLSSTLTLSKADYEKHKVYACEVTHQGLSSPV |
| | TKSFNRGEC |
| SEQ ID NO: 68 | QVQLVESGGGVVQPGRSLRLSCAASGFTFSVYGMNWVRQAPGKGLEWVA |
| anti-IL1b | IIWYDGDNQYYADSVKGRFTISRDNSKNTLYLQMNGLRAEDTAVYYCARD |
| antibody | LRTGPFDYWGQGTLVTVSSASTKGPSVFPLAPSSKSTSGGTAALGCLVKDY |
| (canakinumab) - | FPEPVTVSWNSGALTSGVHTFPAVLQSSGLYSLSSVVTVPSSSLGTQTYICN |
| heavy chain | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 69 | EIVLTQSPDFQSVTPKEKVTITCRASQSIGSSLHWYQQKPDQSPKLLIKYASQ |
| anti-IL1b | SFSGVPSRFSGSGSGTDFTLTINSLEAEDAAAYYCHQSSSLPFTFGPGTKVDI |
| antibody | KRTVAAPSVFIFPPSDEQLKSGTASVVCLLNNFYPREAKVQWKVDNALQSG |
| (canakinumab) - | NSQESVTEQDSKDSTYSLSSTLTLSKADYEKHKVYACEVTHQGLSSPVTKS |
| light chain | FNRGEC |
| SEQ ID NO: 70 | QVQLVESGGGVVQPGRSLRLSCAASGFTFSSYGMHWVRQAPGKGLEWVA |
| anti-IL23 | VIWYDGSNEYYADSVKGRFTISRDNSKNTLYLQMNSLRAEDTAVYYCARD |
| antibody | RGYTSSWYPDAFDIWGQGTMVTVSSASTKGPSVFPLAPCSRSTSESTAALG |
| (brazikumab) - | CLVKDYFPEPVTVSWNSGALTSGVHTFPAVLQSSGLYSLSSVVTVPSSNFG |
| heavy chain | TQTYTCNVDHKPSNTKVDKTVERKCCVECPPCPAPPVAGPSVFLFPPKPKD |
| | TLMISRTPEVTCVVVDVSHEDPEVQFNWYVDGVEVHNAKTKPREEQFNST |
| | FRVVSVLTVVHQDWLNGKEYKCKVSNKGLPAPIEKTISKTKGQPREPQVY |
| | TLPPSREEMTKNQVSLTCLVKGFYPSDIAVEWESNGQPENNYKTTPPMLDS |
| | DGSFFLYSKLTVDKSRWQQGNVFSCSVMHEALHNHYTQKSLSLSPGK |
| SEQ ID NO: 71 | QSVLTQPPSVSGAPGQRVTISCTGSSSNTGAGYDVHWYQQVPGTAPKLLIY |
| anti-IL23 | GSGNRPSGVPDRFSGSKSGTSASLAITGLQAEDEADYYCQSYDSSLSGWVF |
| antibody | GGGTRLTVLGQPKAAPSVTLFPPSSEELQANKATLVCLISDFYPGAVTVAW |
| (brazikumab) - | KADSSPVKAGVETTTPSKQSNNKYAASSYLSLTPEQWKSHRSYSCQVTHE |
| light chain | GSTVEKTVAPTECS |
| SEQ ID NO: 72 | EVQLVQSGAEVKKPGESLKISCKGSGYSFSNYWIGWVRQMPGKGLEWMGI |
| anti-IL23 | IDPSNSYTRYSPSFQGQVTISADKSISTAYLQWSSLKASDTAMYYCARWYY |
| antibody | KPFDVWGQGTLVTVSSASTKGPSVFPLAPSSKSTSGGTAALGCLVKDYFPE |
| (guselkumab) - | PVTVSWNSGALTSGVHTFPAVLQSSGLYSLSSVVTVPSSSLGTQTYICNVNH |
| heavy chain | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 73 | QSVLTQPPSVSGAPGQRVTISCTGSSSNIGSGYDVHWYQQLPGTAPKLLIYG |
| anti-IL23 | NSKRPSGVPDRFSGSKSGTSASLAITGLQSEDEADYYCASWTDGLSLVVFG |
| antibody | GGTKLTVLGQPKAAPSVTLFPPSSEELQANKATLVCLISDFYPGAVTVAWK |
| (guselkumab) - | ADSSPVKAGVETTTPSKQSNNKYAASSYLSLTPEQWKSHRSYSCQVTHEGS |
| light chain | TVEKTVAPTECS |
| SEQ ID NO: 74 | QVQLVQSGAEVKKPGSSVKVSCKASGYTFTDQTIHWMRQAPGQGLEWIG |
| anti-IL23 | YIYPRDDSPKYNENFKGKVTITADKSTSTAYMELSSLRSEDTAVYYCAIPDR |
| antibody | SGYAWFIYWGQGTLVTVSSASTKGPSVFPLAPSSKSTSGGTAALGCLVKDY |
| (risankizumab) - | FPEPVTVSWNSGALTSGVHTFPAVLQSSGLYSLSSVVTVPSSSLGTQTYICN |
| heavy chain | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPG |
| SEQ ID NO: 75 | DIQMTQSPSSLSASVGDRVTITCKASRDVAIAVAWYQQKPGKVPKLLIYWA |
| anti-IL23 | STRHTGVPSRFSGSGSRTDFTLTISSLQPEDVADYFCHQYSSYPFTFGSGTKL |
| antibody | EIKRTVAAPSVFIFPPSDEQLKSGTASVVCLLNNFYPREAKVQWKVDNALQ |
| (risankizumab) - | SGNSQESVTEQDSKDSTYSLSSTLTLSKADYEKHKVYACEVTHQGLSSPVT |
| light chain | KSFNRGEC |
| SEQ ID NO: 76 | QVQLVQSGAEVKKPGASVKVSCKASGYIFITYWMTWVRQAPGQGLEWM |
| anti-IL23 | GQIFPASGSADYNEKFEGRVTMTTDTSTSTAYMELRSLRSDDTAVYYCAR |
| antibody | GGGGFAYWGQGTLVTVSSASTKGPSVFPLAPSSKSTSGGTAALGCLVKDY |
| (tildrakizumab) - | FPEPVTVSWNSGALTSGVHTFPAVLQSSGLYSLSSVVTVPSSSLGTQTYICN |
| heavy chain | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 77 | DIQMTQSPSSLSASVGDRVTITCRTSENIYSYLAWYQQKPGKAPKLLIYNAK |
| anti-IL23 | TLAEGVPSRFSGSGSGTDFTLTISSLQPEDFATYYCQHHYGIPFTFGQGTKVE |
| antibody | IKRTVAAPSVFIFPPSDEQLKSGTASVVCLLNNFYPREAKVQWKVDNALQS |
| (tildrakizumab) - | GNSQESVTEQDSKDSTYSLSSTLTLSKADYEKHKVYACEVTHQGLSSPVTK |
| light chain | SFNRGEC |
| SEQ ID NO: 78 | QVQLQESGPGLVRPSQTLSLTCTVSGYSITSDHAWSWVRQPPGRGLEWIGY |
| anti-IL6R | ISYSGITTYNPSLKSRVTMLRDTSKNQFSLRLSSVTAADTAVYYCARSLART |
| antibody | TAMDYWGQGSLVTVSSASTKGPSVFPLAPSSKSTSGGTAALGCLVKDYFPE |
| (tocilizumab) - | PVTVSWNSGALTSGVHTFPAVLQSSGLYSLSSVVTVPSSSLGTQTYICNVNH |
| heavy chain | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 79 | DIQMTQSPSSLSASVGDRVTITCRASQDISSYLNWYQQKPGKAPKLLIYYTS |
| anti-IL6R | RLHSGVPSRFSGSGSGTDFTFTISSLQPEDIATYYCQQGNTLPYTFGQGTKV |
| antibody | EIKRTVAAPSVFIFPPSDEQLKSGTASVVCLLNNFYPREAKVQWKVDNALQ |
| (tocilizumab) - | SGNSQESVTEQDSKDSTYSLSSTLTLSKADYEKHKVYACEVTHQGLSSPVT |
| light chain | KSFNRGEC |
| SEQ ID NO: 80 | DVQLQESGPGLVKPSQSLSLTCSVTGYSITSGYYWNWIRQFPGNKLEWMG |
| anti-LAP | YISYDGTNNYNPSLKNRISITRDTSKHQFFLKLNSVTTEDTATYYCARSFYN |
| antibody (7H4) - | NYFDFWGQGTTLTVSSASTKGPSVFPLAPSSKSTSGGTAALGCLVKDYFPE |
| heavy chain | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 81 | DIQMTOSPSSLSASLGGKVTITCKASODIDKYIAWYQHKPGKGPRLLIHYTS |
| anti-LAP | TLQPGIPSRFSGSGSGRDYSFNISNLEPEDIATYYCLQYDNLRTFGGGTKLEI |
| antibody (7H4) - | KRTVAAPSVFIFPPSDEQLKSGTASVVCLLNNFYPREAKVQWKVDNALQSG |
| light chain | NSQESVTEQDSKDSTYSLSSTLTLSKADYEKHKVYACEVTHQGLSSPVTKS |
| | FNRGEC |
| SEQ ID NO: 82 | QVQLVQSGAEVKKPGASVKVSCKASGLTIEDYYMHWVRQAPGQGLEWM |
| anti-LIV-1 | GWIDPENGDTEYGPKFQGRVTMTRDTSINTAYMELSRLRSDDTAVYYCAV |
| antibody | HNAHYGTWFAYWGQGTLVTVSSASTKGPSVFPLAPSSKSTSGGTAALGCL |
| (ladiratuzumab) - | VKDYFPEPVTVSWNSGALTSGVHTFPAVLQSSGLYSLSSVVTVPSSSLGTQT |
| heavy chain | YICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPG |
| SEQ ID NO: 83 | DVVMTQSPLSLPVTLGQPASISCRSSQSLLHSSGNTYLEWYQQRPGQSPRPL |
| anti-LIV-1 | IYKISTRFSGVPDRFSGSGSGTDFTLKISRVEAEDVGVYYCFQGSHVPYTFG |
| antibody | GGTKVEIKRTVAAPSVFIFPPSDEQLKSGTASVVCLLNNFYPREAKVQWKV |
| (ladiratuzumab) - | DNALQSGNSQESVTEQDSKDSTYSLSSTLTLSKADYEKHKVYACEVTHQG |
| light chain | LSSPVTKSFNRGEC |
| SEQ ID NO: 84 | EVQLVQSGAEVKKPGSSVKVSCKASGFTFTDYYIHWVKQAPGQGLEWIGL |
| anti-LRRC15 | VYPYIGGTNYNQKFKGKATLTVDTSTTTAYMEMSSLRSEDTAVYYCARGD |
| an (ABBV- | NKYDAMDYWGQGTTVTVSSASTKGPSVFPLAPSSKSTSGGTAALGCLVKD |
| 085- | YFPEPVTVSWNSGALTSGVHTFPAVLQSSGLYSLSSVVTVPSSSLGTQTYIC |
| huAD208.4.1) - | NVNHKPSNTKVDKKVEPKSCDKTHTCPPCPAPELLGGPSVFLFPPKPKDTL |
| heavy chain | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| SEQ ID NO: 85 | FFLYSKLTVDKSRWQQGNVFSCSVMHEALHNHYTQKSLSLSPGK |
| anti-LRRC15 | DIVLTQSPDSLAVSLGERATINCRASQSVSTSSYSYMHWYQQKPGQPPKLLI |
| an (ABBV- | KYASSLESGVPDRFSGSGSGTDFTLTISSLQAEDVAVYYCEQSWEIRTFGGG |
| 085- | TKVEIKRTVAAPSVFIFPPSDEQLKSGTASVVCLLNNFYPREAKVQWKVDN |
| huAD208.4.1) - | ALQSGNSQESVTEQDSKDSTYSLSSTLTLSKADYEKHKVYACEVTHQGLSS |
| light chain | PVTKSFNRGEC |
| SEQ ID NO: 86 | EVQLVQSGAEVKKPGASVKVSCKASGYKFSSYWIEWVKQAPGQGLEWIG |
| anti-LRRC15 | EILPGSDTTNYNEKFKDRATFTSDTSINTAYMELSRLRSDDTAVYYCARDR |
| antibody (ABBV- | GNYRAWFGYWGQGTLVTVSSASTKGPSVFPLAPSSKSTSGGTAALGCLVK |
| 085-huM25) - | DYFPEPVTVSWNSGALTSGVHTFPAVLQSSGLYSLSSVVTVPSSSLGTQTYI |
| heavy chain | CNVIVHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| SEQ ID NO: 87 | DIQMTQSPSSLSASVGDRVTITCRASQDISNYLNWYQQKPGGAVKFLIYYTS |
| anti-LRRC15 | RLHSGVPSRFSGSGSGTDYTLTISSLQPEDFATYFCQQGEALPWTFGGGTKV |
| antibody (ABBV- | EIKRTVAAPSVFIFPPSDEQLKSGTASVVCLLNNFYPREAKVQWKVDNALQ |
| 085-huM25) - | SGNSQESVTEQDSKDSTYSLSSTLTLSKADYEKHKVYACEVTHQGLSSPVT |
| light chain | KSFNRGEA |
| SEQ ID NO: 88 | QVQLQQSGAEVKKFGASVKVSCEASGYTFPSYVLHWVKQAPGQGLEWIG |
| anti-MUC1 | YINPYNDGTQYNKKFKGKATLTRDTSINTAYMELSRLRSDDTAVYYCARG |
| antibody | FGGSYGFAYWGQGTLVIVSSASTKGPSVFPLAPSSKSTSGGTAALGCLVKD |
| (clivatuzumab) - | YFPEPVTVSWNSGALTSGVHTFPAVLQSSGLYSLSSVVTVPSSSLGTQTYIC |
| heavy chain | NVNHKPSNTKVDKRVEPKSCDKTHTCPPCPAPELLGGPSVFLFPPKPKDTL |
| | MISRTPEVTCVVVDVSHEDPEVKFNWYVDGVEVHNAKTKPRESQYNSTYR |
| | VVSVLTVLHQDWLNGKEYKCKVSNEALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVNHEALHNHYTQKSLSLSPGK |
| SEQ ID NO: 89 | DIQLTQSPSSLSASVGDRVTMTCSASSSVSSSYLYWYQQKPGKAPKLWIYS |
| anti-MUC1 | TSNLASGVPARFSGSGSGTDFTLTISSLQPEDSASYFCHQWNRYPYTFGGGT |
| antibody | RLEIKRTVAAPSVFIFPPSDEQLKSGTASVVCLLNNFYPREAKVQWKVDNA |
| (clivatuzumab) - | LQSGNSQESVTEQDSKDSTYSLSSTLTLSKADYEKHKVYACEVTHQGLSSP |
| light chain | VTKSFNRGEC |
| SEQ ID NO: 90 | EVQLQESGPGLVKPSDTLSLTCAVSGYSIASGYYWNWIRQPPGKGLEWMG |
| anti-OX40 | YISYDGSNNYNPSLGNRITISRDTSKNQVSLKLSSVTAVDTAVYYCVKTLPY |
| antibody (ABBV- | YFDYWGQGTTVTVSSASTKGPSVFPLAPSSKSTSGGTAALGCLVKDYFPEP |
| 368-Hu3726) - | VTVSWNSGALTSGVHTFPAVLQSSGLYSLSSVVTVPSSSLGTQTYICNVNH |
| heavy chain | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 91 | DIQMTQTPSSLSASVGDRVTITCRASQDISNYLNWYQQKPGKAPKLLIFYTS |
| anti-OX40 | RLHSGVPSRFSGSGSGTDYTLTISSLQPEDFATYYCQQGNTLPLTFGQGTKL |
| antibody (ABBV- | EIKRTVAAPSVFIFPPSDEQLKSGTASVVCLLNNFYPREAKVQWKVDNALQ |
| 368-Hu3726) - | SGNSQESVTEQDSKDSTYSLSSTLTLSKADYEKHKVYACEVTHQGLSSPVT |
| light chain | KSFNRGEC |
| | EVQLVQSGAEVKKPGSSVKVSCKASGFNIKDTYMHWVRQAPGQGLEWIG |
| SEQ ID NO: 92 | RIDPANGNTKYDPKFQGRATITADTSTNTAYMELSSLRSEDTAVYYCARGG |
| anti-OX40 | PAWFVYWGQGTLVTVSSASTKGPSVFPLAPSSKSTSGGTAALGCLVKDYFP |
| antibody (ABBV- | EPVTVSWNSGALTSGVHTFPAVLQSSGLYSLSSVVTVPSSSLGTQTYICNVN |
| 368-Hu3739) - | HKPSNTKVDKKVEPKSCDKTHTCPPCPAPELLGGPSVFLFPPKPKDTLMISR |
| heavy chain | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 93 | DIQMTQSPSSLSASVGDRVTITCRASQDISNYLNWYQQKPGKAPKLLIYYTS |
| anti-OX40 | RLRSGLPSRFSGSGSGTDYTLTISSLQPEDFATYYCQQGNTLPWTFGGGTKV |
| antibody (ABBV- | EIKRTVAAPSVFIFPPSDEQLKSGTASVVCLLNNFYPREAKVQWKVDNALQ |
| 368-Hu3739) - | SGNSQESVTEQDSKDSTYSLSSTLTLSKADYEKHKVYACEVTHQGLSSPVT |
| light chain | KSFNRGEC |
| SEQ ID NO: 94 | EVQLVESGGGLVQPGGSLRLSCAVSGFSLTSYGVHWVRQAPGKGLEWLG |
| anti-OX40 | VIWSGGSTDYNAAFISRLTISKDNSKSTVYLQMNSLRAEDTAVYYCAREEF |
| antibody (ABBV- | DYWGQGTTVTVSSASTKGPSVFPLAPSSKSTSGGTAALGCLVKDYFPEPVT |
| 368-Hu3741) - | VSWNSGALTSGVHTFPAVLQSSGLYSLSSVVTVPSSSLGTQTYICNVNHKPS |
| heavy chain | NTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| SEQ ID NO: 95 | DIQMTQSPSSLSASVGDRVTITCRASQDISNYLNWFQQKPGKAPKLLIYYTS |
| anti-OX40 | RLHSGVPSRFSGSGSGTDYTLTISSLQPEDFATYYCQQGYTLPPTFGGGTKV |
| antibody (ABBV- | EIKRTVAAPSVFIFPPSDEQLKSGTASVVCLLNNFYPREAKVQWKVDNALQ |
| 368-Hu3741) - | SGNSQESVTEQDSKDSTYSLSSTLTLSKADYEKHKVYACEVTHQGLSSPVT |
| light chain | KSFNRGEC |
| SEQ ID NO: 96 | QVQLVQSGSELKKPGASVKVSCKASGYTFTDYSMHWVRQAPGQGLKWM |
| anti-OX40 | GWINTETGEPTYADDFKGRFVFSLDTSVSTAYLQISSLKAEDTAVYYCANP |
| antibody | YYDYVSYYAMDYWGOGTTVTVSSASTKGPSVFPLAPSSKSTSGGTAALGC |
| (GSK3174998) - | LVKDYFPEPVTVSWNSGALTSGVHTFPAVLQSSGLYSLSSVVTVPSSSLGTQ |
| heavy chain | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| SEQ ID NO: 97 | DIQMTQSPSSLSASVGDRVTITCKASQDVSTAVAWYQQKPGKAPKLLIYSA |
| anti-OX40 | SYLYTGVPSRFSGSGSGTDFTFTISSLQPEDIATYYCOQHYSTPRTFGQGTKL |
| antibody | EIKRTVAAPSVFIFPPSDEQLKSGTASVVCLLNNFYPREAKVQWKVDNALQ |
| (GSK3174998) - | SGNSQESVTEQDSKDSTYSLSSTLTLSKADYEKHKVYACEVTHQGLSSPVT |
| light chain | KSFNRGEC |
| SEQ ID NO: 98 | EVQLVQSGAEVKKPGESLRISCKGSGYTFTTYWMHWVRQATGQGLEWMG |
| anti-PD1 | NIYPGTGGSNFDEKFKNRVTITADKSTSTAYMELSSLRSEDTAVYYCTRWT |
| antibody | TGTGAYWGQGTTVTVSSASTKGPSVFPLAPCSRSTSESTAALGCLVKDYFP |
| (spartalizumab) - | EPVTVSWNSGALTSGVHTFPAVLQSSGLYSLSSVVTVPSSSLGTKTYTCNV |
| heavy chain | DHKPSNTKVDKRVESKYGPPCPPCPAPEFLGGPSVFLFPPKPKDTLMISRTP |
| | EVTCVVVDVSQEDPEVQFNWYVDGVEVHNAKTKPREEQFNSTYRVVSVL |
| | TVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYS |
| | RLTVDKSRWQEGNVFSCSVMHEALHNHYTQKSLSLSLG |
| SEQ ID NO: 99 | EIVLTQSPATLSLSPGERATLSCKSSQSLLDSGNQKNFLTWYQQKPGQAPRL |
| anti-PD1 | LTYWASTRESGVPSRFSGSGSGTDFTFTISSLEAEDAATYYCQNDYSYPYTF |
| antibody | GQGTKVEIKRTVAAPSVFIFPPSDEQLKSGTASVVCLLNNFYPREAKVQWK |
| (spartalizumab) - | VDNALQSGNSQESVTEQDSKDSTYSLSSTLTLSKADYEKHKVYACEVTHQ |
| light chain | GLSSPVTKSFNRGEC |
| SEQ ID NO: 100 | QVQLVQSGVEVKKPGASVKVSCKASGYTFTNYYMYWVRQAPGQGLEWM |
| anti-PD1 | GGINPSNGGTNFNEKFKNRVTLTTDSSTTTAYMELKSLQFDDTAVYYCARR |
| antibody | DYRFDMGFDYWGQGITVTVSSASTKGPSVFPLAPCSRSTSESTAALGCLVK |
| (pembrolizumab) - | DYFPEPVTVSWNSGALTSGVHTFPAVLQSSGLYSLSSVVTVPSSSLGTKTYT |
| heavy chain | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGK |
| SEQ ID NO: 101 | EIVLTQSPATLSLSPGERATLSCRASKGVSTSGYSYLHWYQQKPGQAPRLLI |
| anti-PD1 | YLASYLESGVPARFSGSGSGTDFTLTISSLEPEDFAVYYCQHSRDLPLTFGG |
| antibody | GTKVEIKRTVAAPSVFIFPPSDEQLKSGTASVVCLLNNFYPREAKVQWKVD |
| (pembrolizumab) - | NALQSGNSQESVTEQDSKDSTYSLSSTLTLSKADYEKHKVYACEVTHQGLS |
| light chain | SPVTKSFNRGEC |
| SEQ ID NO: 102 | EIQLVQSGAEVKKPGSSVKVSCKASGYTFTHYGMNWVRQAPGQGLEWVG |
| anti-PD1 | WVNTYTGEPTYADDFKGRLTFTLDTSTSTAYMELSSLRSEDTAVYYCTRE |
| antibody (ABBV- | GEGLGFGDWGQGTTVTVSSASTKGPSVFPLAPSSKSTSGGTAALGCLVKDY |
| 181) - heavy chain | FPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 103 | DVVMTQSPLSLPVTPGEPASISCRSSQSIVHSHGDTYLEWYLQKPGQSPQLL |
| anti -PD1 | IYKVSNRFSGVPDRFSGSGSGTDFTLKISRVEAEDVGVYYCFQGSHIPVTFG |
| antibody (ABBV. - | QGTKLEIKRTVAAPSVFIFPPSDEQLKSGTASVVCLLNNFYPREAKVQWKV |
| 181) - light chain | DNALQSGNSQESVTEQDSKDSTYSLSSTLTLSKADYEKHKVYACEVTHQG |
| | LSSPVTKSFNRGEC |
| SEQ ID NO: 104 | QVQLVESGGGVVQPGRSLRLDCKASGITFSNSGMHWVRQAPGKGLEWVA |
| anti-PD1 | VIWYDGSKRYYADSVKGRFTISRDNSKNTLFLQMNSLRAEDTAVYYCATN |
| antibody | DDYWGQGTLVTVSSASTKGPSVFPLAPCSRSTSESTAALGCLVKDYFPEPV |
| (nivolumab) - | TVSWNSGALTSGVHTFPAVLQSSGLYSLSSVVTVPSSSLGTKTYTCNVDHK |
| heavy chain | PSNTKVDKRVESKYGPPCPPCPAPEFLGGPSVFLFPPKPKDTLMISRTPEVTC |
| | VVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTV |
| | DKSRWQEGNVFSCSVMHEALHNHYTQKSLSLSLGK |
| SEQ ID NO: 105 | EIVLTQSPATLSLSPGERATLSCRASQSVSSYLAWYQQKPGQAPRLLIYDAS |
| anti-PD1 | NRATGIPARFSGSGSGTDFTLTISSLEPEDFAVYYCQQSSNWPRTFGQGTKV |
| antibody | EIKRTVAAPSVFIFPPSDEQLKSGTASVVCLLNNFYPREAKVQWKVDNALQ |
| (nivolumab) - | SGNSQESVTEQDSKDSTYSLSSTLTLSKADYEKHKVYACEVTHQGLSSPVT |
| light chain | KSFNRGEC |
| SEQ ID NO: 106 | EVTLRESGPALVKPTQTLTLTCTFSGFSLSTYGMGVGWIRQPPGKALEWLA |
| anti-PDGF | NIWWDDDKYYNPSLKNRLTISKDTSKNQVVLTMTNMDPVDTATYYCARIE |
| antibody (ab1) - | SSGPKYSFDYWGQGTMVTVSSASTKGPSVFPLAPSSKSTSGGTAALGCLVK |
| heavy chain | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 107 | EIVLTOSPGTLSLSPGERATLSCRASSGSIWYSFVSWYOQKPGQAPRLLIYA |
| anti-PDGF | DDQRASGIPDRFSGSGSGTDFTLTISRLEPEDFAVYYCQSYGINIDVVFGGGT |
| antibody (ab1) - | KVEIKRTVAAPSVFIFPPSDEQLKSGTASVVCLLNNFYPREAKVQWKVDNA |
| light chain | LQSGNSQESVTEQDSKDSTYSLSSTLTLSKADYEKHKVYACEVTHQGLSSP |
| | VTKSFNRGEC |
| SEQ ID NO: 108 | EVQLVESGGGLVQPGGSLRLSCAASGFTFSDSWIHWVRQAPGKGLEWVA |
| anti-PDL1 | WISPYGGSTYYADSVKGRFTISADTSKNTAYLQMNSLRAEDTAVYYCARR |
| antibody | HWPGGFDYWGQGTLVTVSAASTKGPSVFPLAPSSKSTSGGTAALGCLVKD |
| (atezolizumab) - | YFPEPVTVSWNSGALTSGVHTFPAVLQSSGLYSLSSVVTVPSSSLGTQTYIC |
| heavy chain | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| SEQ ID NO: 109 | DIQMTQSPSSLSASVGDRVTITCRASQDVSTAVAWYQQKPGKAPKLLIYSA |
| anti-PDL1 | SFLYSGVPSRFSGSGSGTDFTLTISSLQPEDFATYYCQQYLYHPATFGQGTK |
| antibody | VEIKRTVAAPSVFIFPPSDEQLKSGTASVVCLLNNFYPREAKVQWKVDNAL |
| (atezolizumab) - | QSGNSQESVTEQDSKDSTYSLSSTLTLSKADYEKHKVYACEVTHQGLSSPV |
| light chain | TKSFNRGEC |
| SEQ ID NO: 110 | EVQLLESGGGLVQPGGSLRLSCAASGFTFSSYIMMWVRQAPGKGLEWVSSI |
| anti-PDL1 | YPSGGITFYADTVKGRFTISRDNSKNTLYLQMNSLRAEDTAVYYCARIKLG |
| antibody | TVTTVDYWGQGTLVTVSSASTKGPSVFPLAPSSKSTSGGTAALGCLVKDYF |
| (avelumab) - | PEPVTVSWNSGALTSGVHTFPAVLQSSGLYSLSSVVTVPSSSLGTQTYICNV |
| heavy chain | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| SEQ ID NO: 111 | QSALTQPASVSGSPGQSITISCTGTSSDVGGYNYVSWYQQHPGKAPKLMIY |
| anti-PDL1 | DVSNRPSGVSNRFSGSKSGNTASLTISGLQAEDEADYYCSSYTSSSTRVFGT |
| antibody | GTKVTVLGQPKANPTVTLFPPSSEELQANKATLVCLISDFYPGAVTVAWKA |
| (avelumab) - light | DGSPVKAGVETTKPSKQSNNKYAASSYLSLTPEQWKSHRSYSCQVTHEGS |
| chain | TVEKTVAPTECS |
| SEQ ID NO: 112 | EVQLVESGGGLVQPGGSLRLSCAASGFTFSRYWMSWVRQAPGKGLEWVA |
| anti-PDL1 | NIKQDGSEKYYVDSVKGRFTISRDNAKNSLYLQMNSLRAEDTAVYYCARE |
| antibody | GGWFGELAFDYWGQGTLVTVSSASTKGPSVFPLAPSSKSTSGGTAALGCL |
| (durvalumab) - | VKDYFPEPVTVSWNSGALTSGVHTFPAVLQSSGLYSLSSVVTVPSSSLGTQT |
| heavy chain | YICNVNHKPSNTKVDKRVEPKSCDKTHTCPPCPAPEFEGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPASIEKTISKAKGQPREPQVY |
| | TLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| SEQ ID NO: 113 | EIVLTQSPGTLSLSPGERATLSCRASQRVSSSYLAWYQQKPGQAPRLLIYDA |
| anti-PDL1 | SSRATGIPDRFSGSGSGTDFTLTISRLEPEDFAVYYCQQYGSLPWTFGQGTK |
| antibody | VEIKRTVAAPSVFIFPPSDEQLKSGTASVVCLLNNFYPREAKVQWKVDNAL |
| (durvalumab) - | QSGNSQESVTEQDSKDSTYSLSSTLTLSKADYEKHKVYACEVTHQGLSSPV |
| light chain | TKSFNRGEC |
| SEQ ID NO: 114 | EVQLVQSGAEVKKPGSSVKVSCKASGYTFTTYWMHWVRQAPGQGLEWIG |
| anti-PRLR | EIDPSDSYSNYNQKFKDRATLTVDKSTSTAYMELSSLRSEDTAVYYCARNG |
| antibody (ab1) - | GLGPAWFSYWGQGTLVTVSSASTKGPSVFPLAPSSKSTSGGTAALGCLVKD |
| heavy chain | YFPEPVTVSWNSGALTSGVHTFPAVLQSSGLYSLSSVVIVPSSSLGTQTYICN |
| | VNHKPSNTKVDKKVEPKSCDKTHTCPPCPAPELLGGPCVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 115 | DIQMTQSPSSVSASVGDRVTITCKASQYVGTAVAWYQQKPGKSPKLLIYSA |
| anti-PRLR | SNRYTGVPSRFSDSGSGTDFTLTISSLQPEDFATYFCQQYSSYPWTFGGGTK |
| antibody (ab1) - | VEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPV |
| light chain | TKSFNRGEC |
| SEQ ID NO: 116 | QLQESGPGLVKPSQTLSLTCTVSGGSISSGGYYWSWIRQHPGKGLEWIGYIY |
| anti-PSCA | YSGSTYYNPSLKSRVTISVDTSKNQFSLKLSSGTAADTAVYYCARDHITMV |
| antibody (ab1) - | RGVPKGMDVWGQGTTVTVSSASTKGPSVFPLAPSSKSTSGGTAALGCLVK |
| heavy chain | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 117 | QLTQSPSSLSASVGDRVTITCRASQSISRHLNWYQQKPGKAPKFLIYVASSL |
| anti-PSCA | QSGVPSRFSGSGSGTDFTLTISSLQPEDFATYFCQQSYSIPRTFGQGTKVEIK |
| antibody (ab1) - | RTVAAPSVFIFPPSDEQLKSGTASVVCLLNNFYPREAKVQWKVDNALQSGN |
| light chain | SQESVTEQDSKDSTYSLSSTLTLSKADYEKHKVYACEVTHQGLSSPVTKSF |
| | NRGEC |
| SEQ ID NO: 118 | QVQLVESGGGVVQPGRSLRLSCAASGFTFSNYVMHWVRQAPGKGLEWVA |
| anti-PSMA | IIWYDGSNKYYADSVKGRFTISRDNSKNTLYLQMNSLRAEDTAVYYCAGG |
| antibody (ab2) - | YNWNYEYHYYGMDVWGQGTTVTVSSASTKGPSVFPLAPSSKSTSGGTAA |
| heavy chain | LGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | K |
| SEQ ID NO: 119 | DIQMTQSPSSLSASVGDRVTITCRASQGITNYLAWFQQKPGKAPKSLIYAAS |
| anti-PSMA | SLQSGVPSKFSGSGSGTDFSLTISSLQPEDFATYYCQQYNSYPITFGQGTRLE |
| antibody (ab2) - | IKRTVAAPSVFIFPPSDEQLKSGTASVVCLLNNFYPREAKVQWKVDNALQS |
| light chain | GNSQESVTEQDSKDSTYSLSSTLTLSKADYEKHKVYACEVTHQGLSSPVTK |
| | SFNRGEC |
| SEQ ID NO: 120 | QVQLVESGGGVVQPGRSLRLSCAASGFAFSRYGMHWVRQAPGKGLEWVA |
| anti-PSMA | VIWYDGSNKYYADSVKGRFTISRDNSKNTQYLQMNSLRAEDTAVYYCAR |
| antibody (ab1) - | GGDFLYYYYYGMDVWGQGTTVTVSSASTKGPSVFPLAPSSKSTSGGTAAL |
| heavy chain | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | K |
| SEQ ID NO: 121 | DIQMTQSPSSLSASVGDRVTITCRASQGISNYLAWYQQKTGKVPKFLIYEAS |
| anti-PSMA | TLQSGVPSRFSGGGSGTDFTLTISSLQPEDVATYYCQNYNSAPFTFGPGTKV |
| antibody (ab1) - | DIKRTVAAPSVFIFPPSDEQLKSGTASVVCLLNNFYPREAKVQWKVDNALQ |
| light chain | SGNSQESVTEQDSKDSTYSLSSTLTLSKADYEKHKVYACEVTHQGLSSPVT |
| | KSFNRGEC |
| SEQ ID NO: 122 | QVQLVQSGPEVKKPGASVKVSCKASGYTFTDYAVHWVRQAPGKRLEWIG |
| anti-PTK7 | VISTYNDYTYNNQDFKGRVTMTRDTSASTAYMELSRLRSEDTAVYYCARG |
| antibody | NSYFYALDYWGQGTSVTVSSASTKGPSVFPLAPSSKSTSGGTAALGCLVKD |
| (hSC6.24) - heavy | YFPEPVTVSWNSGALTSGVHTFPAVLQSSGLYSLSSVVTVPSSSLGTQTYIC |
| chain | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| SEQ ID NO: 123 | EIVLTQSPATLSLSPGERATLSCRASESVDSYGKSFMHWYQQKPGQAPRLLI |
| anti-PTK7 | YRASNLESGIPARFSGSGSGTDFTLTISSLEPEDFAVYYCQQSNEDPWTFGG |
| antibody | GTKLEIKRTVAAPSVFIFPPSDEQLKSGTASVVCLLNNFYPREAKVQWKVD |
| (hSC6.24) - light | NALQSGNSQESVTEQDSKDSTYSLSSTLTLSKADYEKHKVYACEVTHQGLS |
| chain | SPVTKSFNRGEC |
| SEQ ID NO: 124 | EVQLVQSGAEVKKPGESLKISCKGSGYSFTSSWINWVRQMPGKGLEWMGR |
| anti-SEZ6 | IYPGEGDTNYSGNFEGQVTISADKSISTAYLQWSSLKASDTAMYYCTRGLV |
| antibody | MDYWGQGTLVTVSSASTKGPSVFPLAPSSKSTSGGTAALGCLVKDYFPEPV |
| (hSC17.200) - | TVSWNSGALTSGVHTFPAVLQSSGLYSLSSVVTVPSSSLGTQTYICNVNHKP |
| heavy chain | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ ID NO: 125 | EIVLTQSPATLSLSPGERATLSCRASQSVDYNGISYMHWYQQKPGQAPRLLI |
| anti-SEZ6 | YAASNVQSGIPARFSGSGSGTDFTLTISSLEPEDFAVYYCQQSIEDPPTFGGG |
| antibody | TKVEIKRTVAAPSVFIFPPSDEQLKSGTASVVCLLNNFYPREAKVQWKVDN |
| (hSC17.200) - | ALQSGNSQESVTEQDSKDSTYSLSSTLTLSKADYEKHKVYACEVTHQGLSS |
| light chain | PVTKSFNRGEC |
| SEQ ID NO: 126 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| antibody | NYWYFDVWGQGTLVTVSSASTKGPSVFPLAPSSKSTSGGTAALGCLVKDY |
| (elotuzumab) - | FPEPVTVSWNSGALTSGVHTFPAVLQSSGLYSLSSVVTVPSSSLGTQTYICN |
| heavy chain | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 127 | DIQMTQSPSSLSASVGDRVTITCKASQDVGIAVAWYQQKPGKVPKLLIYWA |
| anti-SLAMF7 | STRHTGVPDRFSGSGSGTDFTLTISSLQPEDVATYYCQQYSSYPYTFGQGTK |
| antibody | VEIKRTVAAPSVFIFPPSDEQLKSGTASVVCLLNNFYPREAKVQWKVDNAL |
| (elotuzumab) - | QSGNSQESVTEQDSKDSTYSLSSTLTLSKADYEKHKVYACEVTHQGLSSPV |
| light chain | TKSFNRGEC |
| SEQ ID NO: 128 | EVQLLESGGGLVQPGGSLRLSCAASGFTFSNYAMSWVRQAPGKGLEWVSS |
| anti-TF antibody | ISGSGDYTYYTDSVKGRFTISRDNSKNTLYLQMNSLRAEDTAVYYCARSP |
| (tisotumab) - | WGYYLDSWGQGTLVTVSSASTKGPSVFPLAPSSKSTSGGTAALGCLVKDY |
| heavy chain | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID NO: 129 | DIQMTQSPPSLSASAGDRVTITCRASQGISSRLAWYQQKPEKAPKSLIYAAS |
| anti-TF antibody | SLQSGVPSRFSGSGSGTDFTLTISSLQPEDFATYYCQQYNSYPYTFGQGTKL |
| (tisotumab) - light | EIKRTVAAPSVFIFPPSDEQLKSGTASVVCLLNNFYPREAKVQWKVDNALQ |
| chain | SGNSQESVTEQDSKDSTYSLSSTLTLSKADYEKHKVYACEVTHQGLSSPVT |
| | KSFNRGEC |
| SEQ ID NO: 130 | QVQLVQSGAEVKKPGSSVKVSCKASGGTFSSYAISWVRQAPGQGLEWMG |
| anti-TGFb | GIIPIFGTANYAQKFQGRVTITADESTSTAYMELSSLRSEDTAVYYCARGLW |
| antibody (XOMA- | EVRALPSVYWGQGTLVTVSSASTKGPSVFPLAPSSKSTSGGTAALGCLVKD |
| 089) - heavy chain | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| SEQ ID NO: 131 | SYELTQPPSVSVAPGQTARITCGANDIGSKSVHWYQQKAGQAPVLVVSEDI |
| anti-TGFb | IRPSGIPERISGSNSGNTATLTISRVEAGDEADYYCQVWDRDSDQYVFGTGT |
| antibody (XOMA- | KVTVLGQPKAAPSVTLFPPSSEELQANKATLVCLISDFYPGAVTVAWKADS |
| 089) - light chain | SPVKAGVETTTPSKQSNNKYAASSYLSLTPEQWKSHRSYSCQVTHEGSTVE |
| | KTVAPTECS |
| SEQ ID NO: 132 | QVQLVQSGAEVKKPGSSVKVSCKASGYTFSSNVISWVRQAPGQGLEWMG |
| anti-TGFb | GVIPIVDIANYAQRFKGRVTITADESTSTTYMELSSLRSEDTAVYYCASTLG |
| antibody | LVLDAMDYWGQGTLVTVSSASTKGPSVFPLAPCSRSTSESTAALGCLVKD |
| (fresolimumab) - | YFPEPVTVSWNSGALTSGVHTFPAVLQSSGLYSLSSVVTVPSSSLGTKTYTC |
| heavy chain | NVDHKPSNTKVDKRVESKYGPPCPSCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGK |
| SEQ ID NO: 133 | ETVLTQSPGTLSLSPGERATLSCRASQSLGSSYLAWYQQKPGQAPRLLIYGA |
| anti-TGFb | SSRAPGIPDRFSGSGSGTDFTLTISRLEPEDFAVYYCQQYADSPITFGQGTRL |
| antibody | EIKRTVAAPSVFIFPPSDEQLKSGTASVVCLLNNFYPREAKVQWKVDNALQ |
| (fresolimumab) - | SGNSQESVTEQDSKDSTYSLSSTLTLSKADYEKHKVYACEVTHQGLSSPVT |
| light chain | KSFNRGEC |
| SEQ ID NO: 134 | QLQVQESGPGLVKPSETLSLTCTVSGGSISNSYFSWGWIRQPPGKGLEWIGS |
| anti-TGFbRII | FYYGEKTYYNPSLKSRATISIDTSKSQFSLKLSSVTAADTAVYYCPRGPTMI |
| antibody | RGVIDSWGQGTLVTVSSASTKGPSVFPLAPSSKSTSGGTAALGCLVKDYFP |
| (LY3022859) - | EPVTVSWNSGALTSGVHTFPAVLQSSGLYSLSSVVTVPSSSLGTQTYICNVN |
| heavy chain | HKPSNTKVDKRVEPKSCDKTHTCPPCPAPEAEGAPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPSSIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 135 | EIVLTQSPATLSLSPGERATLSCRASQSVRSYLAWYQQKPGQAPRLLIYDAS |
| anti-TGFbRII | NRATGIPARFSGSGSGTDFTLTISSLEPEDFAVYYCQQRSNWPPTFGQGTKV |
| antibody | EIKRTVAAPSVFIFPPSDEQLKSGTASVVCLLNNFYPREAKVQWKVDNALQ |
| (LY3022859) - | SGNSQESVTEQDSKDSTYSLSSTLTLSKADYEKFDVYACEVTHQGLSSPVT |
| light chain | KSFNRGEC |
| SEQ ID NO: 136 | QVQLQESGPGLVKPSETLSLTCAVSGYSITSDYAWNWIRQPPGKGLEWIGY |
| anti-TIGIT | ISYSGSTSYNPSLRSRVTISRDTSKNQFFLKLSSVTAADTAVYYCARRQVGL |
| antibody | GFAYWGQGTLVTVSSASTKGPSVFPLAPSSKSTSGGTAALGCLVKDYFPEP |
| (etigilimab) - | VTVSWNSGALTSGVHTFPAVLQSSGLYSLSSVVTVPSSSLGTQTYICNVNH |
| heavy chain | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 137 | DIQMTQSPSSLSASVGDRVTITCKASQDVSTAVAWYQQKPGKAPKLLIYSA |
| anti-TIGIT | SYRYTGVPSRFSGSGSGTDFTFTISSLQPEDIATYYCQQHYSTPWTFGQGTK |
| antibody | VEIKRTVAAPSVFIFPPSDEQLKSGTASVVCLLNNFYPREAKVQWKVDNAL |
| (etigilimab) - light | QSGNSQESVTEQDSKDSTYSLSNTLTLSKADYEKHKVYACEVTHQGLSSPV |
| chain | TKSFNRGEC |
| SEQ ID NO: 138 | EVQLQQSGPGLVKPSQTLSLTCAISGDSVSSNSAAWNWIRQSPSRGLEWLG |
| anti-TIGIT | KTYYRFKWYSDYAVSVKGRITINPDTSKNQFSLQLNSVTPEDTAVFYCTRE |
| antibody | STTYDLLAGPFDYWGQGTLVTVSSASTKGPSVFPLAPSSKSTSGGTAALGC |
| (tiragolumab) - | LVKDYFPEPVTVSWNSGALTSGVHTFPAVLQSSGLYSLSSVVTVPSSSLGTQ |
| heavy chain | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| SEQ ID NO: 139 | DIVMTQSPDSLAVSLGERATINCKSSQTVLYSSNNKKYLAWYQQKPGQPPN |
| anti-TIGIT | LLIYWASTRESGVPDRFSGSGSGTDFTLTISSLQAEDVAVYYCQQYYSTPFT |
| antibody | FGPGTKVEIKRTVAAPSVFIFPPSDEQLKSGTASVVCLLNNFYPREAKVQW |
| (tiragolumab) - | KVDNALQSGNSQESVTEQDSKDSTYSLSSTLTLSKADYEKHKVYACEVTH |
| light chain | QGLSSPVTKSFNRGEC |
| SEQ ID NO: 140 | QSALTQPRSVSGSPGQSVTISCTGTSSDVGGYNYVSWYQQHPGKAPKLMIY |
| anti-TIM3 | DVSKRPSGVPDRFSGSKSGNTASLTISGLQAEDEADYYCSSYADSVVFGGG |
| antibody (M6903) | TKVTVLGQPKAAPSVTLFPPSSEELQANKATLVCLISDFYPGAVTVAWKAD |
| - heavy chain | SSPVKAGVETTTPSKQSNNKYAASSYLSLTPEQWKSHKSYSCQVTHEGSTV |
| | EKTVAPTECS |
| SEQ ID NO: 141 | EVQLVESGGGLVQPGGSLRLSCAASGFTFSSYAMSWVRQAPGKGLEWVSA |
| anti-TIM3 | ISVSGGSTYYADSVKGRFTISRDNSKNTLYLQMNSLRAEDTAVYYCAKAN |
| antibody (M6903) | WGFFDYWGQGTLVTVSSASTKGPSVFPLAPSSKSTSGGTAALGCLVKDYFP |
| - light chain | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 142 | EVQLVESGGGLVQPGGSLRLSCAASGYDFTHYGMNWVRQAPGKGLEWV |
| anti-VEGF | GWINTYTGEPTYAADFKRRFTFSLDTSKSTAYLQMNSLRAEDTAVYYCAK |
| antibody | YPYYYGTSHWYFDVWGQGTLVTVSSASTKGPSVFPLAPSSKSTSGGTAAL |
| (ranibizumab) - | GCLVKDYFPEPVTVSWNSGALTSGVHTFPAVLQSSGLYSLSSVVTVPSSSL |
| heavy chain | GTQTYICNVNHKPSNTKVDKKVEPKSCDKTHLCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | K |
| SEQ ID NO: 143 | DIQLTQSPSSLSASVGDRVTITCSASQDISNYLNWYQQKPGKAPKVLIYFTSS |
| anti-VEGF | LHSGVPSRFSGSGSGTDFTLTISSLQPEDFATYYCQQYSTVPWTFGQGTKVE |
| antibody | IKRTVAAPSVFIFPPSDEQLKSGTASVVCLLNNFYPREAKVQWKVDNALQS |
| (ranibizumab) - | GNSQESVTEQDSKDSTYSLSSTLTLSKADYEKHKVYACEVTHQGLSSPVTK |
| light chain | SFNRGEC |
| SEQ ID NO: 144 | EVQLVESGGGLVQPGGSLRLSCAASGYTFTNYGMNWVRQAPGKGLEWVG |
| anti-VEGF | WINTYTGEPTYAADFKRRFTFSLDTSKSTAYLQMNSLRAEDTAVYYCAKY |
| antibody | PHYYGSSHWYFDVWGQGTLVTVSSASTKGPSVFPLAPSSKSTSGGTAALG |
| (bevacizumab) - | CLVKDYFPEPVTVSWNSGALTSGVHTFPAVLQSSGLYSLSSVVTVPSSSLGT |
| heavy chain | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| SEQ ID NO: 145 | EVQLVESGGGLVQPGGSLRLSCAASGFTFSNFPMAWVRQAPGKGLEWVAT |
| anti-VEGF | ISSSDGTTYYRDSVKGRFTISRDNAKNSLYLQMNSLRAEDTAVYYCARGY |
| antibody (ABT- | YNSPFAYWGQGTLVTVSSASTKGPSVFPLAPSSKSTSGGTAALGCLVKDYF |
| 165) - heavy chain | PEPVTVSWNSGALTSGVHIFPAVLQSSGLYSLSSVVTVPSSSLGTQTYICNV |
| | NHKPSNTKVDKKVEPKSCDKTHTCPPCPAPEAAGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| SEQ ID NO: 146 | DIQMTQSPSSLSASVGDRVTITCRASEDIYSNLAWYQQKPGKAPKLLIYDTN |
| anti-VEGF | NLADGVPSRFSGSGSGTDFTLTISSLQPEDFATYYCQQYNNYPPTFGQGTKL |
| antibody (ABT- | EIKRTVAAPSVFIFPPSDEQLKSGTASVVCLLNNFYPREAKVQWKVDNALQ |
| 165) - light chain | SGNSQESVTEQDSKDSTYSLSSTLTLSKADYEKHKVYACEVTHQGLSSPVT |
| | KSFNRGEC |
| SEQ ID NO: 147 | EVQLVQSGGGLVKPGGSLRLSCAASGFTFSSYSMNWVRQAPGKGLEWVSS |
| anti-VEGFR | ISSSSSYIYYADSVKGRFTISRDNAKNSLYLQMNSLRAEDTAVYYCARVTD |
| antibody | AFDIWGQGTMVTVSSASTKGPSVFPLAPSSKSTSGGTAALGCLVKDYFPEP |
| (ramucirumab) - | VTVSWNSGALTSGVHTFPAVLQSSGLYSLSSVVTVPSSSLGTQTYICNVNH |
| heavy chain | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 148 | DIQMTQSPSSVSASIGDRVTITCRASQGIDNWLGWYQQKPGKAPKLLIYDA |
| anti-VEGFR | SNLDTGVPSRFSGSGSGTYFTLTISSLQAEDFAVYFCQQAKAFPPTFGGGTK |
| antibody | VDIKGTVAAPSVFIFPPSDEQLKSGTASVVCLLNNFYPREAKVQWKVDNAL |
| (ramucirumab) - | QSGNSQESVTEQDSKDSTYSLSSTLTLSKADYEKHKVYACEVTHQGLSSPV |
| light chain | TKSFNRGEC |
| SEQ ID NO: 149 | QVQLVQSGAEVKKPGSSVKVSCKASGGTFSSYAISWVRQAPGQGLEWMG |
| anti-VISTA | GIIPIFGTANYAQKFQGRVTITADESTSTAYMELSSLRSEDTAVYYCARSSY |
| antibody | GWSYEFDYWGQGTLVTVSSASTKGPSVFPLAPSSKSTSGGTAALGCLVKD |
| (onvatilimab) - | YFPEPVTVSWNSGALTSGVHTFPAVLQSSGLYSLSSVVTVPSSSLGTQTYIC |
| heavy chain | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| SEQ ID NO: 150 | DIQMTQSPSSLSASVGDRVTITCRASQSIDTRLNWYQQKPGKAPKLLIYSAS |
| anti-VISTA | SLQSGVPSRFSGSGSGTDFTLTISSLQPEDFATYYCQQSAYNPITFGQGTKVE |
| antibody | IKRTVAAPSVFIFPPSDEQLKSGTASVVCLLNNFYPREAKVQWKVDNALQS |
| (onvatilimab) - | GNSQESVTEQDSKDSTYSLSSTLTLSKADYEKHKVYACEVTHQGLSSPVTK |
| light chain | SFNRGEC |
| SEQ ID NO: 151 | QVQLVQSGAEVKKPGASVKVSCKASGYIFTAYTMHWVRQAPGQGLEWM |
| anti-cMet | GWIKPNNGLANYAQKFQGRVTMTRDTSISTAYMELSRLRSDDTAVYYCAR |
| antibody | SEITTEFDYWGQGTLVTVSSASTKGPSVFPLAPSSKSTSGGTAALGCLVKDY |
| (telisotuzumab) - | FPEPVTVSWNSGALTSGVHTFPAVLQSSGLYSLSSVVTVPSSSLGTQTYICN |
| heavy chain | VNHKPSNTKVDKRVEPKSCDCHCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPG |
| SEQ ID NO: 152 | DIVMTQSPDSLAVSLGERATINCKSSESVDSYANSFLHWYQQKPGQPPKLLI |
| anti-cMet | YRASTRESGVPDRFSGSGSGTDFTLTISSLQAEDVAVYYCQQSKEDPLTFGG |
| antibody | GTKVEIKRTVAAPSVFIFPPSDEQLKSGTASVVCLLNNFYPREAKVQWKVD |
| (telisotuzumab) - | NALQSGNSQESVTEQDSKDSTYSLSSTLTLSKADYEKHKVYACEVTHQGLS |
| light chain | SPVTKSFNRGEC |
| SEQ ID NO: 153 | QVQLVQSGAEVKKPGASVKVSCKASGYTFTEYTLHWVRQAPGQRLEWM |
| anti-claudin | GGINPNNGDTIYNQKFKGRVTITRDTSASTAYMELSSLRSEDTAVYYCARR |
| antibody | AITVYAMDYWGQGTLVTVSSASTKGPSVFPLAPSSKSTSGGTAALGCLVK |
| (hSC27.1) - heavy | DYFPEPVTVSWNSGALTSGVHTFPAVLQSSGLYSLSSVVTVPSSSLGTQTYI |
| chain | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 154 | DIQMTQSPSSVSASVGDRVTITCKASEDIYNRLAWYQQKPGKAPKLLIYGA |
| anti-claudin | TSLETGVPSRFSGSGSGTDYTLTISSLQPEDFATYYCQQYWSTPLTFGQGTK |
| antibody | LEIKRTVAAPSVFIFPPSDEQLKSGTASVVCLLNNFYPREAKVQWKVDNAL |
| (hSC27. 1) - light | QSGNSQESVTEQDSKDSTYSLSSTLTLSKADYEKHKVYACEVTHQGLSSPV |
| chain | TKSFNRGEC |
| SEQ ID NO: 155 | QVQLVQSGAEVKKPGASVKVSCQASGYRFSNFVIHWVRQAPGQRFEWMG |
| anti- gp120 | WINPYNGNKEFSAKFQDRVTFTADTSANTAYMELRSLRSADTAVYYCARV |
| antibody (B12) - | GPYSWDDSPQDNYYMDVWGKGTTVIVSSASTKGPSVFPLAPSSKSTSGGT |
| heavy chain | AALGCLVKDYFPEPVTVSWNSGALTSGVHTFPAVLQSSGLYSLSSVVTVPS |
| | SSLGTQTYICNVNHKPSNTKVDKRVEPKSCDKTHTCPPCPAPELLGGPSVFL |
| | FPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQ |
| | PREPQVYTLPPSREEMTKNQVSLTCLVKGFYSDIAVEWESNGQPENNYKTT |
| | PPVLDSDGSFFLYSKLTVDKSRWQQGNVFSCSVMHEALHNHYTQKSLSLSP |
| | GK |
| SEQ ID NO: 156 | EIVLTQSPGTLSLSPGERATFSCRSSHSIRSRRVAWYQHKPGQAPRLVIHGVS |
| anti-gp120 | NRASGISDRFSGSGSGTDFTLTITRVEPEDFALYYCQVYGASSYTFGQGTKL |
| antibody (B12) - | ERKRTVAAPSVFIFPPSDEQLKSGTASVVCLLNNFYPREAKVQWKVDNALQ |
| light chain | SGNSQESVTEQDSKDSTYSLSSTLTLSKADYEKHKVYACEVTHQGLSSPVT |
| | KSFNRGEC |
| SEQ ID NO: 157 | EVQLVQSGAEVKKPGASVKVSCKASGDTFKRYYVHWVRQAPGQGLEWM |
| anti-mesothelin | GIINPSGVSTTYAQKFQGRVTMTRDTSTSTVYMELSSLRSEDTAVYYCAEV |
| antibody (ABBV- | RGSGFNYFGMDVWGQGTLVTVSSASTKGPSVFPLAPSSKSTSGGTAALGCL |
| 428) - heavy chain | VKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| SEQ ID NO: 158 | SYELTQPPSVSVSPGQTASITCSGDKLGDKYASWYQQKPGQSPVLVIYQDN |
| anti-mesothelin | RRPSGIPERFSGSNSGNTATLTISGTQAMDEADYYCQAWDSDTYVFGTGTK |
| antibody (ABBV- | VTVLGQPKAAPSVTLFPPSSEELQANKATLVCLISDFYPGAVTVAWKADSS |
| 428) - light chain | PVKAGVETTTPSKQSNNKYAASSYLSLTPEQWKSHRSYSCQVTHEGSTVEK |
| | TVAPTECS |
| SEQ ID NO: 159 | EVQLVESGGGLVQPGGSLRLSCAASGFTFSSYNMNWVRQAPGKGLEWVS |
| anti-nectin4 | YISSSSSTIYYADSVKGRFTISRDNAKNSLSLQMNSLRDEDTAVYYCARAY |
| antibody | YYGMDVWGQGTTVTVSSASTKGPSVFPLAPSSKSTSGGTAALGCLVKDYF |
| (enfortumab) - | PEPVTVSWNSGALTSGVHTFPAVLQSSGLYSLSSVVTVPSSSLGTQTYICNV |
| heavy chain | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| SEQ ID NO: 160 | DIQMTQSPSSVSASVGDRVTITCRASQGISGWLAWYQQKPGKAPKFLIYAA |
| anti-nectin4 | STLQSGVPSRFSGSGSGTDFTLTISSLQPEDFATYYCQQANSFPPTFGGGTKV |
| antibody | EIKRTVAAPSVFIFPPSDEQLKSGTASVVCLLNNFYPREAKVQWKVDNALQ |
| (enfortumab) - | SGNSQESVTEQDSKDSTYSLSSTLTLSKADYEKHKVYACEVTHQGLSSPVT |
| light chain | KSFNRGEC |
| SEQ ID NO: 161 | QVQLQQWGAGLLKPSETLSLTCAVYGGSFSGYYWSWIRQPPGKGLEWIGE |
| anti-uPAR | INHSGSTNYNPSLKSRVTISVDTSKNQFSLKLSSVTAADTAVYYCARGRRFG |
| antibody (ab1) - | DFDYWGQGTLVTVSSASTKGPSVFPLAPSSKSTSGGTAALGCLVKDYFPEP |
| heavy chain | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 162 | QPVLTQPPSVSVAPGKTARITCGGNNIGSKSVHWYQQKPGQAPVLVVYDD |
| anti-uPAR | SDRPPGIPERFSGSNSGNTATLTISRVEAGDEADYYCQVWDSSSDHSPFGTG |
| antibody (ab1) - | TKVTVLGQPKANPTVTLFPPSSEELQANKATLVCLISDFYPGAVTVAWKAD |
| light chain | GSPVKAGVETTKPSKQSNNKYAASSYLSLTPEQWKSHRSYSCQVTHEGST |
| | VEKTVAPTECS |
| SEQ ID NO: 163 | QVQLQQSGPGLVKPSQTLSLTCAISGDSVSSNSAAWNWIRQSPSRGLEWLG |
| anti-uPAR | RTYYRSKWYNDYAVSVKSRIIINPDTSKNQFSLQLNSVTPEDTAVYYCARD |
| antibody (ab2) - | PGGPLDDSFDIWGQGTMVTVSSASTKGPSVFPLAPSSKSTSGGTAALGCLV |
| heavy chain | KDYFPEPVTVSWNSGALTSGVHTFPAVLQSSGLYSLSSVVTVPSSSLGTQTY |
| | ICNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| SEQ ID NO: 164 | LDVVMTQSPLSLPVTPGEPASISCRSSQSLLRSNGYNYLDWYLQKPGQSPQ |
| anti-uPAR | LLIYLGSIRASGVPDRFSGSGSGTDFTLRISRVEAEDVGVYYCMQALQTPFT |
| antibody (ab2) - | FGQGTKLEIKRTVAAPSVFIFPPSDEQLKSGTASVVCLLNNFYPREAKVQW |
| light chain | KVDNALQSGNSQESVTEQDSKDSTYSLSSTLTLSKADYEKHKVYACEVTH |
| | QGLSSPVTKSFNRGEC |
| SEQ ID NO: 165 | IWNIHGKESCDVQLYIKRQSEHSILAGDPFELECPVKYCANRPHVTWCKLN |
| BTLA ECD (25- | GTTCVKLEDRQTSWKEEKNISFFILHFEPVLPNDNGSYRCSANFQSNLIESHS |
| 150) | TTLYVTDVKSASERPSKDEMAS |
| SEQ ID NO: 166 | KESCDVQLYIKRQSEHSILAGDPFELECPVKYCANRPHVTWCKLNGTTCVK |
| BTLA ECD (31- | LEDRQTSWKEEKNISFFILHFEPVLPNDNGSYRCSANFQSNLIESHSTTLYVT |
| 134) | |
| SEQ ID NO: 167 | KESCDVQLYIKRQSEHSILAGDPFELECPVKYCANRPHVTWCKLNGTTCVK |
| BTLA ECD (31- | LEDRQTSWKEEKNISFFILHFEPVLPNDNGSYRCSANFQSNLIESHSTTLYVT |
| 150) | DVKSASERPSKDEMAS |
| SEQ ID NO: 168 | KESCDVQLYIKRQSEHSILAGDPFELECPVKYCANRPHVTWCKLNGTTCVK |
| BTLA ECD (31- | LEDRQTSWKEEKNISFFILHFEPVLPNDNGSYRCSANFQSNLIESHSTTLYVT |
| 157) | DVKSASERPSKDEMASRPWLLYR |
| SEQ ID NO: 169 | PGWFLDSPDRPWNPPTFSPALLVVTEGDNATFTCSFSNTSESFVLNWYRMS |
| PD1 ECD | PSNQTDKLAAFPEDRSQPGQDCRFRVTQLPNGRDFHMSVVRARRNDSGTY |
| | LCGAISLAPKAQIKESLRAELRVTERRAEVPTAHPSPSPRPAGQFQTLV |
| SEQ ID NO: 170 | DSPDRPWNPPTFSPALLVVTEGDNATFTCSFSNTSESFHVVWHRESPSGQTD |
| PD1 ECD (HAC- | TLAAFPEDRSQPGQDARFRVTQLPNGRDFHMSVVRARRNDSGTYVCGVIS |
| V) | LAPKIQIKESLRAELRVTER |
| SEQ ID NO: 171 | PGWFLDSPDRPWNPPTFSPALLVVTEGDNATFTCSFSNTSESFHVVWHRES |
| PD1 ECD (HAC- | PSGQTDTLAAFPEDRSQPGQDARFRVTQLPNGRDFHMSVVRARRNDSGTY |
| V_extended) | VCGVISLAPKIQIKESLRAELRVTERRAEVPTAHPSPSPRPAGQFQTLV |
| SEQ ID NO: 172 | MDGRFWIRVQESVMVPEGLCISVPCSFSYPRQDWTGSTPAYGYWFKAVTE |
| SIGLEC10 ECD | TTKGAPVATNHQSREVEMSTRGRFQLTGDPAKGNCSLVIRDAQMQDESQY |
| (17-546) | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLIST |
| SEQ ID NO: 173 | EEEVQIIQPDKSVSVAAGESAILHCTITSLFPVGPIQWFRGAGPARVLIYNQR |
| SIRPa ECD | QGPFPRVTTISETTRRENMDFSISISNITPADAGTYYCIKFRKGSPDTEFKSGA |
| (FD6) | GTELSVRAKPS |
| SEQ ID NO: 174 | EEELQVIQPDKSVLVAAGETATLRCTATSLIPVGPIQWFRGAGPGRELIYNQ |
| SIRPa ECD (31- | KEGHFPRVTTVSDLTKRNNMDFSIRIGNITPADAGTYYCVKFRKGSPDDVE |
| 150) | FKSGAGTELSVRAKPS |
| SEQ ID NO: 175 | EEELQVIQPDKSVLVAAGETATLRCTATSLIPVGPIQWFRGAGPGRELIYNQ |
| SIRPa ECD (31- | KEGHFPRVTTVSDLTKRNNMDFSIRIGNITPADAGTYYCVKFRKGSPDDVE |
| 373) | FKSGAGTELSVRAKPSAPVVSGPAARATPQHTVSFTCESHGFSPRDITLKWF |
| | KNGNELSDFQTNVDPVGESVSYSIHSTAKVVLTREDVHSQVICEVAHVTLQ |
| | GDPLRGTANLSETIRVPPTLEVTQQPVRAENQVNVTCQVRKFYPQRLQLTW |
| | LENGNVSRTETASTVTENKDGTYNWMSWLLVNVSAHRDDVKLTCQVEHD |
| | GQPAVSKSHDLKVSAHPKEQGSNTAAENTGSNERNIY |
| SEQ ID NO: 176 | EEELQIIQPDKSVLVAAGETATLRCTITSLFPVGPIQWFRGAGPGRVLIYNQR |
| SIRPa ECD | QGPFPRVTTVSDTTKRNNMDFSIRIGNITPADAGTYYCIKFRKGSPDDVEFK |
| (CV1) | SGAGTELSVRAKPS |
| SEQ ID NO: 177 | TIPPHVQKSVNNDMIVTDNNGAVKFPQLCKFCDVRFSTCDNQKSCMSNCSI |
| TGFbR ECD | TSICEKPQEVCVAVWRKNDENITLETVCHDPKLPYHDFILEDAASPKCIMKE |
| (TGFbRII) | KKKPGETFFMCSCSSDECNDNIIFSEEYNTSNPD |
| SEQ ID NO: 178 | IPPHVQKSVNNDMIVTDNNGAVKFPQLCKFCDVRFSTCDNQKSCMSNCSIT |
| TGFbR ECD | SICEKPQEVCVAVWRKNDENITLETVCHDPKLPYHDFILEDAASPKCIMKE |
| (TGFbRII-1) | KKKPGETFFMCSCSSDECNDNIIFSEEYNTSNPD |
| SEQ ID NO: 179 | NGAVKFPQLCKFCDVRFSTCDNQKSCMSNCSITSICEKPQEVCVAVWRKN |
| TGFbR ECD | DENITLETVCHDPKLPYHDFILEDAASPKCIMKEKKKPGETFFMCSCSSDEC |
| (huTGFbRII- | NDNIIFSEEYNTSNPDGLGPVESSPGHGLDTAAAGPEPGALCELSPVSASHP |
| huTGFbRIII- | VQALMESFTVLSGCASRGTTGLPQEVHVLNLRTAGQGPGQLQREVTLHLN |
| huTGFbRII) | PISSVHIHHKSVVFLLNSPHPLVWHLKTERLATGVSRLFLVSEGSVVQFSSA |
| | NFSLTAETEERNFPHGNEHLLNWARKEYGAVTSFTELKIARNIYIKVGEDQ |
| | VFPPKCNIGKNFLSLNYLAEYLQPKAAEGCVMSSQPQNEEVHIIELITPNSNP |
| | YSAFQVDITIDIRPSQEDLEVVKNLILILKCKKSVNWVIKSFDVKGSLKIIAPN |
| | SIGFGKESERSMTMTKSIRDDIPSTQGNLVKWALDNGYSPITSYTMAPVAN |
| | RFHLRLENNAEEMGDEEVHTIPPELRILLDPTCKAQLCKFCDVRFSTCDNQ |
| | KSCMSNCSITSICEKPQEVCVAVWRKNDENITLETVCHDPKLPYHDFILEDA |
| | ASPKCIMKEKKKPGETFFMCSCSSDECNDNIIFSEEYNTSNPD |
| SEQ ID NO: 180 | NGAVKFPQLCKFCDVRFSTCDNQKSCMSNCSITSICEKPQEVCVAVWRKN |
| TGFbR ECD | DENITLETVCHDPKLPYHDFILEDAASPKCIMKEKKKPGETFFMCSCSSDEC |
| (RER) | NDNIIFSEEYNTSNPDGLGPVESSPGHGLDTAAAGPEPSTRCELSPINASHPV |
| | QALMESFTVLSGCASHGTTGLPREVHVLNLRSTDQGPGQRQREVTLHLNPI |
| | ASVHTHHKPIVFLLNSPQPLVWRLKTERLAAGVPRLFLVSEGSVVQFPSGN |
| | FSLTAETEERNFPQENEHLLRWAQKEYGAVTSFTELKIARNIYIKVGEDQVF |
| | PPTCNIGKNFLSLNYLAEYLQPKAAEGCVLPSQPHEKEVHIIELITPSSNPYS |
| | AFQVDIIVDIRPAQEDPEVVKNLVLILKSKKSVNWVIKSFDVKGNLKVIAPN |
| | SIGFGKESERSMTMTKLVRDDIPSTQENLMKWALDAGYRPVTSYTMAPVA |
| | NRFHLRLENNEEMRDEEVHTIPPELRILLDPDKLPQLCKFCDVRFSTCDNQK |
| | SCMSNCSITSICEKPQEVCVAVWRKNDENITLETVCHDPKLPYHDFILEDAA |
| | SPKCIMKEKKKPGETFFMCSCSSDECNDNIIFSEEYNTSNPD |
| SEQ ID NO: 181 | SEVEYRAEVGQNAYLPCFYTPAAPGNLVPVCWGKGACPVFECGNVVLRT |
| TIM3 ECD | DERDVNYWTSRYWLNGDFRKGDVSLTIENVTLADSGIYCCRIQIPGIMNDE |
| | KFNLKLVIKPAKVTPAPTRQRDFTAAFPRMLTTRGHGPAETQTLGSLPDINL |
| | TQISTLANELRDSRLANDLRDSGATIRIG |
| SEQ ID NO: 182 | SEVEYRAEVGQNAYLPCFYTPAAPGNLVPVCWGKGACPVFECGNVVLRT |
| TIM3 ECD | DERDVNYWTSRYWLNGDFRKGDVSLTIENVILADSGIYCCRIQIPGIMNDE |
| (hypoglycosylated | KFNLKLVIKPAKVTPAPTRQRDFTAAFPRMLTTRGHGPAETQTLGSLPDINL |
| TIM3 (T80I, | IQISTLANELRDSRLANDLRDSGATIRIG |
| T153I)) | |
| SEQ ID NO: 183 | SEVEYRAEVGQNAYLPCFYTPAAPGNLVPVCWGKGACPVFECGNVVLRT |
| TIM3 ECD | DERDVNYWTSRYWLNGDFRKGDVSLTIENVILADSGIYCCRIQIPGIMNDE |
| (hypoglycosylated | KFNLKLVIKPAKVTPAPTRQRDFTAAFPRMLTTRGHGPAETQTLGSLPDINL |
| TIM3 (T80I) | TQISTLANELRDSRLANDLRDSGATIRIG |
| SEQ ID NO: 184 | ASVGLPSVSLDLPRLSIQKDILTIKANTTLQITCRGQRDLDWLWPNNQSGSE |
| VEGFR ECD | QRVEVTECSDGLFCKTLTIPKVIGNDTGAYKCFYRETDLASVIYVYVQDYR |
| (VEGFR2) | SPFIASVSDQHGVVYITENKNKTVVIPCLGSISNLNVSLCARYPEKRFVPDG |
| | NRISWDSKKGFTIPSYMISYAGMVFCEAKINDESYQSIMYIVVVVGYRIYDV |
| | VLSPSHGIELSVGEKLVLNCTARTELNVGIDFNWEYPSSKHQHKKLVNRDL |
| | KTQSGSEMKKFLSTLTIDGVTRSDQGLYTCAASSGLMTKKNSTFVRVHEKP |
| | FVAFGSGMESLVEATVGERVRIPAKYLGYPPPEIKWYKNGIPLESNHTIKAG |
| | HVLTIMEVSERDTGNYTVILTNPISKEKQSHVVSLVVYVPPQIGEKSLISPVD |
| | SYQYGTTQTLTCTVYAIPPPHHIHWYWQLEEECANEPSQAVSVTNPYPCEE |
| | WRSVEDFQGGNKIEVNKNQFALIEGKNKTVSTLVIQAANVSALYKCEAVN |
| | KVGRGERVISFHVTRGPEITLQPDMQPTEQESVSLWCTADRSTFENLTWYK |
| | LGPQPLPIHVGELPTPVCKNLDTLWKLNATMFSNSTNDILIMELKNASLQD |
| | QGDYVCLAQDRKTKKRHCVVRQLTVLERVAPTITGNLENQTTSIGESIEVS |
| | CTASGNPPPQIMWFKDNETLVEDSGIVLKDGNRNLTIRRVRKEDEGLYTCQ |
| | ACSVLGCAKVEAFFIIEGAQEKTNLE |
| SEQ ID NO: 185 | SKLKDPELSLKGTQHIMQAGQTLHLQCRGEAAHKWSLPEMVSKESERLSIT |
| VEGFR ECD | KSACGRNGKQFCSTLTLNTAQANHTGFYSCKYLAVPTSKKKETESAIYIFIS |
| (VEGFR1) | DTGRPFVEMYSEIPEIIHMTEGRELVIPCRVTSPNITVTLKKFPLDTLIPDGKR |
| | IIWDSRKGFIISNATYKEIGLLTCEATVNGHLYKTNYLTHRQTNTIIDVQISTP |
| | RPVKLLRGHTLVLNCTAFIPLNTRVQMTWSYPDEKNKRASVRRRIDQSNS |
| | HANIFYSVLTIDKMQNKDKGLYTCRVRSGPSFKSVNTSVHIYDKAFITVKH |
| | RKQQVLETVAGKRSYRLSMKVKAFPSPEVVWLKDGLPATEKSARYLTRG |
| | YSLIIKDVTEEDAGNYTILLSIKQSNVFKNLTATLIVNVKPQIYEKAVSSFPD |
| | PALYPLGSRQILTCTAYGIPQPTIKWFWHPCNHNHSEARCDFCSNNEESFIL |
| | DADSNMGNRIESITQRMAIIEGKNKMASTLVVADSRISGIYICIASNKVGTV |
| | GRNISFYITDVPNGFHVNLEKMPTEGEDLKLSCTVNKFLYRDVTWILLRTV |
| | NNRTMHYSISKQKMAITKEHSITLNLTIMNVSLQDSGTYACRARNVYTGEE |
| | ILQKKEITIRDQEAPYLLRNLSDHTVAISSSTTLDCHANGVPEPQITWFKNNH |
| | KIQQEPGIILGPGSSTLFIERVTEEDEGVYHCKATNQKGSVESSAYLTVQGTS |
| | DKSNLE |
| SEQ ID NO: 186 | SDTGRPFVEMYSEIPEIIHMTEGRELVIPCRVTSPNITVTLKKFPLDTLIPDGK |
| VEGFR ECD | RIIWDSRKGFIISNATYKEIGLLTCEATVNGHLYKTNYLTHRQTNTIIDVVLS |
| (aflibercept) | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEK |
| SEQ ID NO: 187 | EKATKRNDEECEVQLNIKRNSKHSAWTGELFKIECPVKYCVHRPNVTWCK |
| mBTLA ECD | HNGTIWVPLEVGPQLYTSWEENRSVPVFVLHFKPIHLSDNGSYSCSTNFNS |
| (30-176) | QVINSHSVTIHVRERTQNSSEHPLIISDIPDATNASGPSTMEKRPG |
| SEQ ID NO: 188 | ACPWAVSGARASPGSAASPRLREGPELSPDDPAGLLDLRQGMFAQLVAQN |
| 41BBL ECD (50- | VLLIDGPLSWYSDPGLAGVSLTGGLSYKEDTKELVVAKAGVYYVFFQLEL |
| 254) | RRVVAGEGSGSVSLALHLQPLRSAAGAAALALTVDLPPASSEARNSAFGFQ |
| | GRLLHLSAGQRLGVHLHTEARARHAWQLTQGATVLGLFRVTPEIPAGLPSP |
| | RSE |
| SEQ ID NO: 189 | QRTDSIPNSPDNVPLKGGNCSEDLLCILKRAPFKKSWAYLQVAKHLNKTKL |
| CD30L ECD (63- | SWNKDGILHGVRYQDGNLVIQFPGLYFIICQLQFLVQCPNNSVDLKLELLIN |
| 234) | KHIKKQALVTVCESGMQTKHVYQNLSQFLLDYLQVNTTISVNVDTFQYIDT |
| | STFPLENVLSIFLYSNSD |
| SEQ ID NO: 190 | HRRLDKIEDERNLHEDFVFMKTIQRCNTGERSLSLLNCEEIKSQFEGFVKDI |
| CD40L ECD (47- | MLNKEETKKENSFEMQKGDQNPQIAAHVISEASSKTTSVLQWAEKGYYTM |
| 261) | SNNLVTLENGKQLTVKRQGLYYIYAQVTFCSNREASSQAPFIASLCLKSPGR |
| | FERILLRAANTHSSAKPCGQQSIHLGGVFELQPGASVFVNVTDPSQVSHGTG |
| | FTSFGLLKL |
| SEQ ID NO: 191 | GDQNPQIAAHVISEASSKTTSVLQWAEKGYYTMSNNLVTLENGKQLTVKR |
| CD40L ECD | QGLYYIYAQVTFCSNREASSQAPFIASLCLKSPGRFERILLRAANTHSSAKPC |
| (116-261) | GQQSIHLGGVFELQPGASVFVNVTDPSQVSHGTGFTSFGLLKL |
| SEQ ID NO: 192 | QRFAQAQQQLPLESLGWDVAELQLNHTGPQQDPRLYWQGGPALGRSFLH |
| CD70 ECD (39- | GPELDKGQLRIHRDGIYMVHIQVTLAICSSTTASRHHPTTLAVGICSPASRSI |
| 193) | SLLRLSFHQGCTIASQRLTPLARGDTLCTNLTGTLLPSRNTDETFFGVQWVR |
| | P |
| SEQ ID NO: 193 | QLETAKEPCMAKFGPLPSKWQMASSEPPCVNKVSDWKLEILQNGLYLIYG |
| GITRL ECD (72- | QVAPNANYNDVAPFEVRLYKNKDMIQTLTNKSKIQNVGGTYELHVGDTID |
| 199) | LIFNSEHQVLKNNTYWGIILLANPQFIS |
| SEQ ID NO: 194 | DTQEKEVRAMVGSDVELSCACPEGSRFDLNDVYVYWQTSESKTVVTYHIP |
| ICOSL ECD (19- | QNSSLENVDSRYRNRALMSPAGMLRGDFSLRLFNVTPQDEQKFHCLVLSQ |
| 258) | SLGFQEVLSVEVTLHVAANFSVPVVSAPHSPSQDELTFTCTSINGYPRPNVY |
| | WINKTDNSLLDQALQNDTVFLNMRGLYDVVSVLRIARTPSVNIGCCIENVL |
| | LQQNLTVGSQTGNDIGERDKITENPVSTGEKNAATWS |
| SEQ ID NO: 195 | IWELKKDVYVVELDWYPDAPGEMVVLTCDTPEEDGITWTLDQSSEVLGSG |
| IL12 ECD (p40- | KTLTIQVKEFGDAGQYTCHKGGEVLSHSLLLLHKKEDGIWSTDILKDQKEP |
| p35) | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMNIALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNAS |
| SEQ ID NO: 196 | NWVNVISDLKKIEDLIQSMHIDATLYTESDVHPSCKVTAMKCFLLELQVISL |
| IL15 ECD (49- | ESGDASIHDTVENLIILANNSLSSNGNVTESGCKECEELEEKNIKEFLQSFVH |
| 162) | IVQMFINTS |
| SEQ ID NO: 197 | LQLHWRLGEMVTRLPDGPAGSWEQLIQERRSHEVNPAAHLTGANSSLTGS |
| (LIGHT ECD (59- | GGPLLWETQLGLAFLRGLSYHDGALVVTKAGYYYIYSKVQLGGVGCPLGL |
| 240) | ASTITHGLYKRTPRYPEELELLVSQQSPCGRATSSSRVWWDSSFLGGVVHL |
| | EAGEKVVVRVLDERLVRLRDGTRSYFGAFMV |
| SEQ ID NO: 198 | DGPAGSWEQLIQERRSHEVNPAAHLTGANSSLTGSGGPLLWETQLGLAFLR |
| LIGHT ECD (74- | GLSYHDGALVVTKAGYYYIYSKVQLGGVGCPLGLASTITHGLYKRTPRYP |
| 240) | EELELLVSQQSPCGRATSSSRVWWDSSFLGGVVHLEAGEKVVVRVLDERL |
| | VRLRDGTRSYFGAFMV |
| SEQ ID NO: 199 | QVSHRYPRIQSIKVQFTEYKKEKGFILTSQKEDEIMKVQNNSVIINCDGFYLI |
| OX40L ECD (51- | SLKGYFSQEVNISLHYQKDEEPLFQLKKVRSVNSLMVASLTYKDKVYLNV |
| 183) | TTDNTSLDDFHVNGGELILIHQNPGEFCVL |
| SEQ ID NO: 200 | GGGGSGGGGSGGGGS |
| flexible linker - | |
| (GGGGS)3 | |
| SEQ ID NO: 201 | GGGGSGGGGSGGGGSGGGGS |
| flexible linker - | |
| (GGGGS)4 | |
| SEQ ID NO: 202 | GSAGSAAGSGEF |
| flexible linker - | |
| waldo 1999 | |
| SEQ ID NO: 203 | KESGSVSSEQLAQFRSLD |
| flexible linker - | |
| bird1988-1 | |
| SEQ ID NO: 204 | EGKSSGSGSESKST |
| flexible linker - | |
| bird1988-2 | |
| SEQ ID NO: 205 | EAAAKEAAAKEAAAK |
| rigid linker - | |
| (EAAAK)3 | |
| SEQ ID NO: 206 | AEAAAKEAAAKEAAAKA |
| rigid linker - | |
| A(EAAAK)3 A | |
| SEQ ID NO: 207 | APAPAPAPAPAPAP |
| rigid linker - | |
| (AP)7 | |
| SEQ ID NO: 208 | ASTKGPSVFPLAPSSKSTSGGTAALGCLVKDYFPEPVTVSWNSGALTSGVH |
| HC constant | TFPAVLQSSGLYSLSSVVTVPSSSLGTQTYICNVNHKPSNTKVDKKVEPKSC |
| region - IgG1 | DKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK |
| SEQ ID NO: 209 | ASTKGPSVFPLAPSSKSTSGGTAALGCLVKDYFPEPVTVSWNSGALTSGVH |
| HC constant | TFPAVLQSSGLYSLSSVVTVPSSSLGTQTYICNVNHKPSNTKVDKKVEPKSC |
| region - | DKTHTCPPCPAPEAAGGPSVFLFPPKPKDTLMISRTPEVTCVVVDVSHEDPE |
| IgG1_L234A_L23 | VKFNWYVDGVEVHNAKTKPREEQYNSTYRVVSVLTVLHQDWLNGKEYK |
| 5A | CKVSNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK |
| SEQ ID NO: 210 | ASTKGPSVFPLAPCSRSTSESTAALGCLVKDYFPEPVTVSWNSGALTSGVHT |
| HC constant | FPAVLQSSGLYSLSSVVTVPSSSLGTKTYTCNVDHKPSNTKVDKRVESKYG |
| region - IgG4 | PPCPSCPAPEFLGGPSVFLFPPKPKDTLMISRTPEVTCVVVDVSQEDPEVQFN |
| | WYVDGVEVHNAKTKPREEQFNSTYRVVSVLTVLHQDWLNGKEYKCKVS |
| | NKGLPSSIEKTISKAKGQPREPQVYTLPPSQEEMTKNQVSLTCLVKGFYPSD |
| | IAVEWESNGQPENNYKTTPPVLDSDGSFFLYSRLTVDKSRWQEGNVFSCSV |
| | MHEALHNHYTQKSLSLSLGK |
| SEQ ID NO: 211 | ASTKGPSVFPLAPCSRSTSESTAALGCLVKDYFPEPVTVSWNSGALTSGVHT |
| HC constant | FPAVLQSSGLYSLSSVVTVPSSSLGTKTYTCNVDHKPSNTKVDKRVESKYG |
| region - | PPCPPCPAPEFLGGPSVFLFPPKPKDTLMISRTPEVTCVVVDVSQEDPEVQFN |
| IgG4_S228P | WYVDGVEVHNAKTKPREEQFNSTYRVVSVLTVLHQDWLNGKEYKCKVS |
| | NKGLPSSIEKTISKAKGQPREPQVYTLPPSQEEMTKNQVSLTCLVKGFYPSD |
| | IAVEWESNGQPENNYKTTPPVLDSDGSFFLYSRLTVDKSRWQQGNVFSCSV |
| | MHEALHNHYTQKSLSLSGK |
| SEQ ID NO: 212 | RTVAAPSVFIFPPSDEQLKSGTASVVCLLNNFYPREAKVQWKVDNALQSGN |
| LC constant | SQESVTEQDSKDSTYSLSSTLTLSKADYEKHKVYACEVTHQGLSSPVTKSF |
| region - kappa | NRGEC |
| SEQ ID NO: 213 | QPKAAPSVTLFPPSSEELQANKATLVCLISDFYPGAVTVAWKADSSPVKAG |
| LC constant | VETTTPSKQSNNKYAASSYLSLTPEQWKSHRSYSCQVTHEGSTVEKTVAPT |
| region - lambda | ECS |
| SEQ ID NO: 214 | DIQLTQSPASLAVSLGQRATISCKASQSVDYDGDSYLNWYQQIPGQPPKLLI |
| anti-CD3/CD19 | YDASNLVSGIPPRFSGSGSGTDFTLNIHPVEKVDAATYHCQQSTEDPWTFG |
| BiTE | GGTKLEIKGGGGSGGGGSGGGGSQVQLQQSGAELVRPGSSVKISCKASGY |
| (blinatumomab) | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKHHHH |
| | HH |
| SEQ ID NO: 215 | QVQLVESGGGLVKPGESLRLSCAASGFTFSDYYMYWVRQAPGKGLEWVA |
| anti-CD3/PSMA | IISDGGYYTYYSDIIKGRFTISRDNAKNSLYLQMNSLKAEDTAVYYCARGFP |
| BiTE | LLRHGAMDYWGQGTLVTVSSGGGGSGGGGSGGGGSDIQMTQSPSSLSASV |
| (pasotuxizumab) | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLHHHH |
| | HH |
| SEQ ID NO: 216 | QVQLVQSGAEVKKPGASVKVSCKASGYTFTEFGMNWVRQAPGQGLEWM |
| anti- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| CrossMab | LVKDYFPEPVTVSWNSGALTSGVHTFPAVLQSSGLYSLSSVVTVPSSSLGTQ |
| (cibisatamab) - | TYICNVNHKPSNTKVDKKVEPKSCDKTHTCPPCPAPEAAGGPSVFLFPPKP |
| HC1 | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| SEQ ID NO: 217 | QVQLVQSGAEVKKPGASVKVSCKASGYTFTEFGMNWVRQAPGQGLEWM |
| anti- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| CrossMab | LVKDYFPEPVTVSWNSGALTSGVHTFPAVLQSSGLYSLSSVVTVPSSSLGTQ |
| (cibisatamab) - | TYICNVNHKPSNTKVDKKVEPKSCDGGGGSGGGGSEVQLLESGGGLVQPG |
| HC2 | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGK |
| SEQ ID NO: 218 | DIQMTQSPSSLSASVGDRVTITCKASAAVGTYVAWYQQKPGKAPKLLIYSA |
| anti- | SYRKRGVPSRFSGSGSGTDFTLTISSLQPEDFATYYCHQYYTYPLFTFGQGT |
| CEACAM5/CD3 | KLEIKRTVAAPSVFIFPPSDEQLKSGTASVVCLLNNFYPREAKVQWKVDNA |
| CrossMab | LQSGNSQESVTEQDSKDSTYSLSSTLTLSKADYEKHKVYACEVTHQGLSSP |
| (cibisatamab) - | VTKSFNRGEC |
| LC1 | |
| SEQ ID NO: 219 | QAVVTQEPSLTVSPGGTVTLTCGSSTGAVTTSNYANWVQEKPGQAFRGLIG |
| anti- | GTNKRAPGTPARFSGSLLGGKAALTLSGAQPEDEAEYYCALWYSNLWVFG |
| CEACAM5/CD3 | GGTKLTVLSSASTKGPSVFPLAPSSKSTSGGTAALGCLVKDYFPEPVTVSW |
| CrossMab | NSGALTSGVHTFPAVLQSSGLYSLSSVVTVPSSSLGTQTYICNVNHKPSNTK |
| (cibisatamab) - | VDKKVEPKSC |
| LC2 | |
| SEQ ID NO: 220 | QVQLKQSGPGLVQPSQSLSITCTVSGFSLTNYGVHWVRQSPGKGLEWLGVI |
| anti-EGFR | WSGGNTDYNTPFTSRLSINKDNSKSQVFFKMNSLQSNDTAIYYCARALTYY |
| fused to BTLA | DYEFAYWGQGTLVTVSAASTKGPSVFPLAPSSKSTSGGTAALGCLVKDYFP |
| ECD | EPVTVSWNSGALTSGVHTFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | HCTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSKESCDVQLYIKRQSEHSILAGDPFELECPVKYCANRPHVTWCKL |
| | NGTTCVKLEDRQTSWKEEKNISFFILHEEPVLPNDNGSYRCSANFQSNLIES |
| | HSTTLYVTDVKSASERPSKDEMAS |
| SEQ ID NO: 221 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to PD1 ECD | RTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSF |
| | NRGECGGGGSGGGGSGGGGSPGWFLDSPDRPWNPPTFSPALLVVTEGDNA |
| | TFTCSFSNTSESFVLNWYRMSPSNQTDKLAAFPEDRSQPGQDCRFRVTQLP |
| | NGRDFHMSVVRARRNDSGTYLCGAISLAPKAQIKESLRAELRVTERRAEVP |
| | TAHPSPSPRPAGQFQTLV |
| SEQ ID NO: 222 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to | RTVAAPSVFIFPPSDEQLKSGTASVVCLLNNFYPREAKVQWKVDNALQSGN |
| SIGLEC10 ECD | SQESVTEQDSKDSTYSLSSTLTLSKADYEKHKVYACEVTHQGLSSPVTKSF |
| | NRGECGGGGSGGGGSGGGGSMDGRFWIRVQESVMVPEGLCISVPCSFSYP |
| | RQDWTGSTPAYGYWFKAVTETTKGAPVATNHQSREVEMSTRGRFQLTGD |
| | PAKGNCSLVIRDAQMQDESQYFFRVERGSYVRYNFMNDGFFLKVTALTQK |
| | PDVYIPETLEPGQPVTVICVFNWAFEECPPPSFSWTGAALSSQGTKPTTSHFS |
| | VLSFTPRPQDHNTDLTCHVDFSRKGVSAQRTVRLRVAYAPRDLVISISRDN |
| | TPALEPQPQGNVPYLEAQKGQFLRLLCAADSQPPATLSWVLQNRVLSSSHP |
| | WGPRPLGLELPGVKAGDSGRYTCRAENRLGSQQRALDLSVQYPPENLRVM |
| | VSQANRTVLENLGNGTSLPVLEGQSLCLVCVTHSSPPARLSWTQRGQVLSP |
| | SQPSDPGVLELPRVQVEHEGEFTCHARHPLGSQHVSLSLSVHYSPKLLGPSC |
| | SWEAEGLHCSCSSQASPAPSLRWWLGEELLEGNSSQDSFEVTPSSAGPWAN |
| | SSLSLHGGLSSGLRLRCEAWNVHGAQSGSILQLPDKKGLIST |
| SEQ ID NO: 223 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to SIRPa | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSEEELQVIQPDKSVLVAAGETATLRCTATSLI |
| | PVGPIQWFRGAGPGRELIYNQKEGHFPRVTTVSDLTKRNNMDFSIRIGNITP |
| | ADAGTYYCVKFRKGSPDDVEFKSGAGTELSVRAKPS |
| SEQ ID NO: 224 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to TIM3 | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSSEVEYRAEVGQNAYLPCFYTPAAPGNLVPV |
| | CWGKGACPVFECGNVVLRTDERDVNYWTSRYWLNGDFRKGDVSLTIENV |
| | TLADSGIYCCRIQIPGIMNDEKFNLKLVIKPAKVTPAPTRQRDFTAAFPRML |
| | TTRGHGPAETQTLGSLPDINLTQISTLANELRDSRLANDLRDSGATIRIG |
| SEQ ID NO: 225 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to PD1 ECD | DYEFAYWGQGTLVTVSAASTKGPSVFPLAPSSKSTSGGTAALGCLVKDYFP |
| | EPVTVSWNSGALTSGVHIFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSPGWFLDSPDRPWNPPTFSPALLVVTEGDNATFTCSFSNTSESFVL |
| | NWYRMSPSNQTDKLAAFPEDRSQPGQDCRFRVTQLPNGRDFHMSVVRAR |
| | RNDSGTYLCGAISLAPKAQIKESLRAELRVTERRAEVPTAHPSPSPRPAGQF |
| | QTLV |
| SEQ ID NO: 226 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to BTLA | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSKESCDVQLYIKRQSEHSILAGDPFELECPVK |
| | YCANRPHVTWCKLNGTTCVKLEDRQTSWKEEKNISFFILHEEPVLPNDNGS |
| | YRCSANFQSNLIESHSTTLYVTDVKSASERPSKDEMAS |
| SEQ ID NO: 227 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to | DYEFAYWGQGTLVTVSAASTKGPSVFPLAPSSKSTSGGTAALGCLVKDYFP |
| SIGLEC10 ECD | EPVTVSWNSGALTSGVHIFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSMDGRFWIRVQESVMVPEGLCISVPCSFSYPRQDWTGSTPAYGY |
| | WFKAVTETTKGAPVATNHQSREVEMSTRGRFQLTGDPAKGNCSLVIRDAQ |
| | MQDESQYFFRVERGSYVRYNFMNDGFFLKVTALTQKPDVYIPETLEPGQP |
| | VTVICVFNWAFEECPPPSFSWTGAALSSQGTKPTTSHFSVLSFTPRPQDHNT |
| | DLTCHVDFSRKGVSAQRTVRLRVAYAPRDLVISISRDNTPALEPQPQGNVP |
| | YLEAQKGQFLRLLCAADSQPPATLSWVLQNRVLSSSHPWGPRPLGLELPGV |
| | KAGDSGRYTCRAENRLGSQQRALDLSVQYPPENLRVMVSQANRTVLENL |
| | GNGTSLPVLEGQSLCLVCVTHSSPPARLSWTQRGQVLSPSQPSDPGVLELPR |
| | VQVEHEGEFTCHARHPLGSQHVSLSLSVHYSPKLLGPSCSWEAEGLHCSCS |
| | SQASPAPSLRWWLGEELLEGNSSQDSFEVTPSSAGPWANSSLSLHGGLSSG |
| | LRLRCEAWNVHGAQSGSILQLPDKKGLIST |
| SEQ ID NO: 228 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to SIRPa | DYEFAYWGQGTLVTVSAASTKGPSVFPLAPSSKSTSGGTAALGCLVKDYFP |
| ECD | EPVTVSWNSGALTSGVHIFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSEEELQVIQPDKSVLVAAGETATLRCTATSLIPVGPIQWFRGAGPG |
| | RELIYNQKEGHFPRVTTVSDLTKRNNMDFSIRIGNITPADAGTYYCVKFRK |
| | GSPDDVEFKSGAGTELSVRAKPS |
| SEQ ID NO: 229 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to TGFbR | DYEFAYWGQGTLVTVSAASTKGPSVFPLAPSSKSTSGGTAALGCLVKDYFP |
| ECD | EPVTVSWNSGALTSGVHIFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSTIPPHVQKSVNNDMIVTDNNGAVKFPQLCKFCDVRFSTCDNQK |
| | SCMSNCSITSICEKPQEVCVAVWRKNDENITLETVCHDPKLPYHDFILEDAA |
| | SPKCIMKEKKKPGETFFMCSCSSDECNDNIIFSEEYNTSNPD |
| SEQ ID NO: 230 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to TIM3 | DYEFAYWGQGTLVTVSAASTKGPSVFPLAPSSKSTSGGTAALGCLVKDYFP |
| ECD | EPVTVSWNSGALTSGVHIFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSSEVEYRAEVGQNAYLPCFYTPAAPGNLVPVCWGKGACPVFECG |
| | NVVLRTDERDVNYWTSRYWLNGDFRKGDVSLTIENVTLADSGIYCCRIQIP |
| | GIMNDEKFNLKLVIKPAKVTPAPTRQRDFTAAFPRMLTTRGHGPAETQTLG |
| | SLPDINLTQISTLANELRDSRLANDLRDSGATIRIG |
| SEQ ID NO: 231 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to VEGFR | DYEFAYWGQGTLVTVSAASTKGPSVFPLAPSSKSTSGGTAALGCLVKDYFP |
| ECD | EPVTVSWNSGALTSGVHIFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSSDTGRPFVEMYSEIPEIIHMTEGRELVIPCRVTSPNITVTLKKFPL |
| | DTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |
| SEQ ID NO: 232 | QVQLQESGPGLVKPSQTLSLTCTVSGYSISSDFAWNWIRQPPGKGLEWMG |
| anti-EGFRvIII | YISYSGNTRYQPSLKSRITISRDTSKNQFFLKLNSVTAADTATYYCVTAGRG |
| HC fused to | FPYWGQGTLVTVSSASTKGPSVFPLAPSSKSTSGGTAALGCLVKDYFPEPV |
| BTLA ECD | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSKESCDVQLYIKRQSEHSILAGDPFELECPVKYCANRPHVTWCKLNGT |
| | TCVKLEDRQTSWKEEKNISFFILHFEPVLPNDNGSYRCSANFQSNLIESHSTT |
| | LYVTDVKSASERPSKDEMAS |
| SEQ ID NO: 233 | DIQMTQSPSSMSVSVGDRVTITCHSSQDINSNIGWLQQKPGKSFKGLIYHGT |
| anti-EGFRvIII | NLDDGVPSRFSGSGSGTDYTLTISSLQPEDFATYYCVQYAQFPWTFGGGTK |
| LC fused to PD1 | LEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSPGWFLDSPDRPWNPPTFSPALLVVTE |
| | GDNATFTCSFSNTSESFVLNWYRMSPSNQTDKLAAFPEDRSQPGQDCRFRV |
| | TQLPNGRDFHMSVVRARRNDSGTYLCGAISLAPKAQIKESLRAELRVTERR |
| | AEVPTAHPSPSPRPAGQFQTLV |
| SEQ ID NO: 234 | DIQMTQSPSSMSVSVGDRVTITCHSSQDINSNIGWLQQKPGKSFKGLIYHGT |
| anti-EGFRvIII | NLDDGVPSRFSGSGSGTDYTLTISSLQPEDFATYYCVQYAQFPWTFGGGTK |
| LC fused to | LEIKRTVAAPSVFIFPPSDEQLKSGTASVVCLLNNFYPREAKVQWKVDNAL |
| SIGLEC10 ECD | QSGNSQESVTEQDSKDSTYSLSSTLTLSKADYEKHKVYACEVTHQGLSSPV |
| | TKSFNRGECGGGGSGGGGSGGGGSMDGRFWIRVQESVMVPEGLCISVPCS |
| | FSYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 235 | DIQMTQSPSSMSVSVGDRVTITCHSSQDINSNIGWLQQKPGKSFKGLIYHGT |
| anti-EGFRvIII | NLDDGVPSRFSGSGSGTDYTLTISSLQPEDFATYYCVQYAQFPWTFGGGTK |
| LC fused to | LEIKRTVAAPSVFIFPPSDEQLKSGTASVVCLLNNFYPREAKVQWKVDNAL |
| SIRPa ECD | QSGNSQESVTEQDSKDSTYSLSSTLTLSKADYEKHKVYACEVTHQGLSSPV |
| | TKSFNRGECGGGGSGGGGSGGGGSEEELQVIQPDKSVLVAAGETATLRCT |
| | ATSLIPVGPIQWFRGAGPGRELIYNQKEGHFPRVTTVSDLTKRNNMDFSIRI |
| | GNITPADAGTYYCVKFRKGSPDDVEFKSGAGTELSVRAKPS |
| SEQ ID NO: 236 | DIQMTQSPSSMSVSVGDRVTITCHSSQDINSNIGWLQQKPGKSFKGLIYHGT |
| anti-EGFRvIII | NLDDGVPSRFSGSGSGTDYTLTISSLQPEDFATYYCVQYAQFPWTFGGGTK |
| LC fused to TIM3 | LEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSSEVEYRAEVGQNAYLPCFYTPAAPGN |
| | LVPVCWGKGACPVFECGNVVLRTDERDVNYWTSRYWLNGDFRKGDVSL |
| | TIENVTLADSGIYCCRIQIPGIMNDEKFNLKLVIKPAKVTPAPTRQRDFTAAF |
| | PRMLTTRGHGPAETQTLGSLPDINLTQISTLANELRDSRLANDLRDSGATIRI |
| | G |
| SEQ ID NO: 237 | QVQLQESGPGLVKPSQTLSLTCTVSGYSISSDFAWNWIRQPPGKGLEWMG |
| anti-EGFRvIII | YISYSGNTRYQPSLKSRITISRDTSKNQFFLKLNSVTAADTATYYCVTAGRG |
| HC fused to PD1 | FPYWGQGTLVTVSSASTKGPSVFPLAPSSKSTSGGTAALGCLVKDYFPEPV |
| ECD | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSPGWFLDSPDRPWNPPTFSPALLVVTEGDNATFTCSFSNTSESFVLNW |
| | YRMSPSNQTDKLAAFPEDRSQPGQDCRFRVTQLPNGRDFHMSVVRARRND |
| | SGTYLCGAISLAPKAQIKESLRAELRVTERRAEVPTAHPSPSPRPAGQFQTL |
| | V |
| SEQ ID NO: 238 | DIQMTQSPSSMSVSVGDRVTITCHSSQDINSNIGWLQQKPGKSFKGLIYHGT |
| anti-EGFRAII | NLDDGVPSRFSGSGSGTDYTLTISSLQPEDFATYYCVQYAQFPWTFGGGTK |
| LC fused to | LEIKRTVAAPSVFIFPPSDEQLKSGTASVVCLLNNFYPREAKVQWKVDNAL |
| BTLA ECD | QSGNSQESVTEQDSKDSTYSLSSTLTLSKADYEKHKVYACEVTHQGLSSPV |
| | TKSFNRGECGGGGSGGGGSGGGGSKESCDVQLYIKRQSEHSILAGDPFELE |
| | CPVKYCANRPHVTWCKLNGTTCVKLEDRQTSWKEEKNISFFILHFEPVLPN |
| | DNGSYRCSANFQSNLIESHSTTLYVTDVKSASERPSKDEMAS |
| SEQ ID NO: 239 | QVQLQESGPGLVKPSQTLSLTCTVSGYSISSDFAWNWIRQPPGKGLEWMG |
| anti-EGFRvIII | YISYSGNTRYQPSLKSRITISRDTSKNQFFLKLNSVTAADTATYYCVTAGRG |
| HC fused to | FPYWGQGTLVTVSSASTKGPSVFPLAPSSKSTSGGTAALGCLVKDYFPEPV |
| SIGLEC10 ECD | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSMDGRFWIRVQESVMVPEGLCISVPCSFSYPRQDWTGSTPAYGYWFK |
| | AVTETTKGAPVATNHQSREVEMSTRGRFQLTGDPAKGNCSLVIRDAQMQD |
| | ESQYFFRVERGSYVRYNFMNDGFFLKVTALTQKPDVYIPETLEPGQPVTVI |
| | CVFNWAFEECPPPSFSWTGAALSSQGTKPTTSHFSVLSFTPRPQDHNTDLTC |
| | HVDFSRKGVSAQRTVRLRVAYAPRDLVISISRDNTPALEPQPQGNVPYLEA |
| | QKGQFLRLLCAADSQPPATLSWVLQNRVLSSSHPWGPRPLGLELPGVKAG |
| | DSGRYTCRAENRLGSQQRALDLSVQYPPENLRVMVSQANRTVLENLGNGT |
| | SLPVLEGQSLCLVCVTHSSPPARLSWTQRGQVLSPSQPSDPGVLELPRVQVE |
| | HEGEFTCHARHPLGSQHVSLSLSVHYSPKLLGPSCSWEAEGLHCSCSSQASP |
| | APSLRWWLGEELLEGNSSQDSFEVTPSSAGPWANSSLSLHGGLSSGLRLRC |
| | EAWNVHGAQSGSILQLPDKKGLIST |
| SEQ ID NO: 240 | QVQLQESGPGLVKPSQTLSLTCTVSGYSISSDFAWNWIRQPPGKGLEWMG |
| anti-EGFRvIII | YISYSGNTRYQPSLKSRITISRDTSKNQFFLKLNSVTAADTATYYCVTAGRG |
| HC fused to | FPYWGQGTLVTVSSASTKGPSVFPLAPSSKSTSGGTAALGCLVKDYFPEPV |
| SIRPa ECD | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSEEELQVIQPDKSVLVAAGETATLRCTATSLIPVGPIQWFRGAGPGREL |
| | IYNQKEGHFPRVTTVSDLTKRNNMDFSIRIGNITPADAGTYYCVKFRKGSPD |
| | DVEFKSGAGTELSVRAKPS |
| SEQ ID NO: 241 | QVQLQESGPGLVKPSQTLSLTCTVSGYSISSDFAWNWIRQPPGKGLEWMG |
| anti-EGFRvIII | YISYSGNTRYQPSLKSRITISRDTSKNQFFLKLNSVTAADTATYYCVTAGRG |
| HC fused to | FPYWGQGTLVTVSSASTKGPSVFPLAPSSKSTSGGTAALGCLVKDYFPEPV |
| TGFbRECD | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSTIPPHVQKSVNNDMIVTDNNGAVKFPQLCKFCDVRFSTCDNQKSCM |
| | SNCSITSICEKPQEVCVAVWRKNDENITLETVCHDPKLPYHDFILEDAASPK |
| | CIMKEKKKPGETFFMCSCSSDECNDNIIFSEEYNTSNPD |
| SEQ ID NO: 242 | QVQLQESGPGLVKPSQTLSLTCTVSGYSISSDFAWNWIRQPPGKGLEWMG |
| anti-EGFRvIII | YISYSGNTRYQPSLKSRITISRDTSKNQFFLKLNSVTAADTATYYCVTAGRG |
| HC fused to TIM3 | FPYWGQGTLVTVSSASTKGPSVFPLAPSSKSTSGGTAALGCLVKDYFPEPV |
| ECD | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSSEVEYRAEVGQNAYLPCFYTPAAPGNLVPVCWGKGACPVFECGNV |
| | VLRTDERDVNYWTSRYWLNGDFRKGDVSLTIENVTLADSGIYCCRIQIPGI |
| | MNDEKFNLKLVIKPAKVTPAPTRQRDFTAAFPRMLTTRGHGPAETQTLGSL |
| | PDINLTQISTLANELRDSRLANDLRDSGATIRIG |
| SEQ ID NO: 243 | QVQLQESGPGLVKPSQTLSLTCTVSGYSISSDFAWNWIRQPPGKGLEWMG |
| anti-EGFRAII | YISYSGNTRYQPSLKSRITISRDTSKNQFFLKLNSVTAADTATYYCVTAGRG |
| HC fused to | FPYWGQGTLVTVSSASTKGPSVFPLAPSSKSTSGGTAALGCLVKDYFPEPV |
| VEGFR ECD | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSG |
| | GGGSSDTGRPFVEMYSEIPEIIHMTEGRELVIPCRVTSPNITVTLKKFPLDTLI |
| | PDGKRIIWDSRKGFIISNATYKEIGLLTCEATVNGHLYKTNYLTHRQTNTIID |
| | VVLSPSHGIELSVGEKLVLNCTARTELNVGIDFNWEYPSSKHQHKKLVNRD |
| | LKTQSGSEMKKFLSTLTIDGVTRSDQGLYTCAASSGLMTKKNSTFVRVHEK |
| SEQ ID NO: 244 | EVQLVESGGGLVQPGGSLRLSCAASGFNIKDTYIHWVRQAPGKGLEWVAR |
| anti-HER2 HC | IYPTNGYTRYADSVKGRFTISADTSKNTAYLQMNSLRAEDTAVYYCSRWG |
| fused to BTLA | GDGFYAMDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSKESCDVQLYIKRQSEHSILAGDPFELECPVKYCANRPHVT |
| | WCKLNGTTCVKLEDRQTSWKEEKNISFFILHFEPVLPNDNGSYRCSANFQS |
| | NLIESHSTTLYVTDVKSASERPSKDEMAS |
| SEQ ID NO: 245 | DIQMTQSPSSLSASVGDRVTITCRASQDVNTAVAWYQQKPGKAPKLLIYSA |
| anti-HER2 LC | SFLYSGVPSRFSGSRSGTDFTLTISSLQPEDFATYYCQQHYTTPPTFGQGTKV |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 246 | DIQMTQSPSSLSASVGDRVTITCRASQDVNTAVAWYQQKPGKAPKLLIYSA |
| anti-HER2 LC | SFLYSGVPSRFSGSRSGTDFTLTISSLQPEDFATYYCQQHYTTPPTFGQGTKV |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 247 | DIQMTQSPSSLSASVGDRVTITCRASQDVNTAVAWYQQKPGKAPKLLIYSA |
| anti-HER2 LC | SFLYSGVPSRFSGSRSGTDFTLTISSLQPEDFATYYCQQHYTTPPTFGQGTKV |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 248 | DIQMTQSPSSLSASVGDRVTITCRASQDVNTAVAWYQQKPGKAPKLLIYSA |
| anti-HER2 LC | SFLYSGVPSRFSGSRSGTDFTLTISSLQPEDFATYYCQQHYTTPPTFGQGTKV |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 249 | EVQLVESGGGLVQPGGSLRLSCAASGFNIKDTYIHWVRQAPGKGLEWVAR |
| anti-HER2 HC | IYPTNGYTRYADSVKGRFTISADTSKNTAYLQMNSLRAEDTAVYYCSRWG |
| fused to PD1 ECD | GDGFYAMDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSPGWFLDSPDRPWNPPTFSPALLVVTEGDNATFTCSFSNTS |
| | ESFVLNWYRMSPSNQTDKLAAFPEDRSQPGQDCRFRVTQLPNGRDFHMSV |
| | VRARRNDSGTYLCGAISLAPKAQIKESLRAELRVTERRAEVPTAHPSPSPRP |
| | AGQFQTLV |
| SEQ ID NO: 250 | DIQMTQSPSSLSASVGDRVTITCRASQDVNTAVAWYQQKPGKAPKLLIYSA |
| anti-HER2 LC | SFLYSGVPSRFSGSRSGTDFTLTISSLQPEDFATYYCQQHYTTPPTFGQGTKV |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 251 | EVQLVESGGGLVQPGGSLRLSCAASGFNIKDTYIHWVRQAPGKGLEWVAR |
| anti-HER2 HC | IYPTNGYTRYADSVKGRFTISADTSKNTAYLQMNSLRAEDTAVYYCSRWG |
| fused to | GDGFYAMDYWGQGTLVTVSSASTKGPSVFPLAPSSKSTSGGTAALGCLVK |
| SIGLEC10 ECD | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSMDGRFWIRVQESVMVPEGLCISVPCSFSYPRQDWTGSTP |
| | AYGYWFKAVTETTKGAPVATNHQSREVEMSTRGRFQLTGDPAKGNCSLVI |
| | RDAQMQDESQYFFRVERGSYVRYNFMNDGFFLKVTALTQKPDVYIPETLE |
| | PGQPVTVICVFNWAFEECPPPSFSWTGAALSSQGTKPTTSHFSVLSFTPRPQ |
| | DHNTDLTCHVDFSRKGVSAQRTVRLRVAYAPRDLVISISRDNTPALEPQPQ |
| | GNVPYLEAQKGQFLRLLCAADSQPPATLSWVLQNRVLSSSHPWGPRPLGL |
| | ELPGVKAGDSGRYTCRAENRLGSQQRALDLSVQYPPENLRVMVSQANRTV |
| | LENLGNGTSLPVLEGQSLCLVCVTHSSPPARLSWTQRGQVLSPSQPSDPGV |
| | LELPRVQVEHEGEFTCHARHPLGSQHVSLSLSVHYSPKLLGPSCSWEAEGL |
| | HCSCSSQASPAPSLRWWLGEELLEGNSSQDSFEVTPSSAGPWANSSLSLHG |
| | GLSSGLRLRCEAWNVHGAQSGSILQLPDKKGLIST |
| SEQ ID NO: 252 | EVQLVESGGGLVQPGGSLRLSCAASGFNIKDTYIHWVRQAPGKGLEWVAR |
| anti-HER2 HC | IYPTNGYTRYADSVKGRFTISADTSKNTAYLQMNSLRAEDTAVYYCSRWG |
| fused to SIRPa | GDGFYAMDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSEEELQVIQPDKSVLVAAGETATLRCTATSLIPVGPIQWFRG |
| | AGPGRELIYNQKEGHFPRVTTVSDLTKRNNMDFSIRIGNITPADAGTYYCV |
| | KFRKGSPDDVEFKSGAGTELSVRAKPS |
| SEQ ID NO: 253 | EVQLVESGGGLVQPGGSLRLSCAASGFNIKDTYIHWVRQAPGKGLEWVAR |
| anti-HER2 HC | IYPTNGYTRYADSVKGRFTISADTSKNTAYLQMNSLRAEDTAVYYCSRWG |
| fused to TGFbR | GDGFYAMDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSTIPPHVQKSVNNDMIVTDNNGAVKFPQLCKFCDVRFSTC |
| | DNQKSCMSNCSITSICEKPQEVCVAVWRKNDENITLETVCHDPKLPYHDFIL |
| | EDAASPKCIMKEKKKPGETFFMCSCSSDECNDNIIFSEEYNTSNPD |
| SEQ ID NO: 254 | EVQLVESGGGLVQPGGSLRLSCAASGFNIKDTYIHWVRQAPGKGLEWVAR |
| anti-HER2 HC | IYPTNGYTRYADSVKGRFTISADTSKNTAYLQMNSLRAEDTAVYYCSRWG |
| fused to TIM3 | GDGFYAMDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSSEVEYRAEVGQNAYLPCFYTPAAPGNLVPVCWGKGACP |
| | VFECGNVVLRTDERDVNYWTSRYWLNGDFRKGDVSLTIENVTLADSGIYC |
| | CRIQIPGIMNDEKFNLKLVIKPAKVTPAPTRQRDFTAAFPRMLTTRGHGPAE |
| | TQTLGSLPDINLTQISTLANELRDSRLANDLRDSGATIRIG |
| SEQ ID NO: 255 | EVQLVESGGGLVQPGGSLRLSCAASGFNIKDTYIHWVRQAPGKGLEWVAR |
| anti-HER2 HC | IYPTNGYTRYADSVKGRFTISADTSKNTAYLQMNSLRAEDTAVYYCSRWG |
| fused to VEGFR | GDGFYAMDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSSDTGRPFVEMYSEIPEIIHMTEGRELVIPCRVTSPNITVTLK |
| | KFPLDTLIPDGKRIIWDSRKGFIISNATYKEIGLLTCEATVNGHLYKTNYLTH |
| | RQTNTIIDVVLSPSHGIELSVGEKLVLNCTARTELNVGIDFNWEYPSSKHQH |
| | KKLVNRDLKTQSGSEMKKFLSTLTIDGVTRSDQGLYTCAASSGLMTKKNS |
| | TFVRVHEK |
| SEQ ID NO: 256 | EVQLVESGGGLVQPGGSLRLSCAASGFTFSSYNMNWVRQAPGKGLEWVS |
| anti-nectin4 HC | YISSSSSTIYYADSVKGRFTISRDNAKNSLSLQMNSLRDEDTAVYYCARAY |
| fused to BTLA | YYGMDVWGQGTTVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSKESCDVQLYIKRQSEHSILAGDPFELECPVKYCANRPHVTWCK |
| | LNGTTCVKLEDRQTSWKEEKNISFFILHEEPVLPNDNGSYRCSANFQSNLIE |
| | SHSTTLYVTDVKSASERPSKDEMAS |
| SEQ ID NO: 257 | DIQMTQSPSSVSASVGDRVTITCRASQGISGWLAWYQQKPGKAPKFLIYAA |
| anti-nectin4 LC | STLQSGVPSRFSGSGSGTDFTLTISSLQPEDFATYYCQQANSFPPTFGGGTKV |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 258 | DIQMTQSPSSVSASVGDRVTITCRASQGISGWLAWYQQKPGKAPKFLIYAA |
| anti-nectin4 LC | STLQSGVPSRFSGSGSGTDFTLTISSLQPEDFATYYCQQANSFPPTFGGGTKV |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 259 | DIQMTQSPSSVSASVGDRVTITCRASQGISGWLAWYQQKPGKAPKFLIYAA |
| anti-nectin4 LC | STLQSGVPSRFSGSGSGTDFTLTISSLQPEDFATYYCQQANSFPPTFGGGTKV |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 260 | DIQMTQSPSSVSASVGDRVTITCRASQGISGWLAWYQQKPGKAPKFLIYAA |
| anti-nectin4 LC | STLQSGVPSRFSGSGSGTDFTLTISSLQPEDFATYYCQQANSFPPTFGGGTKV |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 261 | EVQLVESGGGLVQPGGSLRLSCAASGFTFSSYNMNWVRQAPGKGLEWVS |
| anti-nectin4 HC | YISSSSSTIYYADSVKGRFTISRDNAKNSLSLQMNSLRDEDTAVYYCARAY |
| fused to PD1 ECD | YYGMDVWGQGTTVTVSSASTKGPSVFPLAPSSKSTSGGTAALGCLVKDYF |
| | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSPGWFLDSPDRPWNPPTFSPALLVVTEGDNATFTCSFSNTSESFV |
| | LNWYRMSPSNQTDKLAAFPEDRSQPGQDCRFRVTQLPNGRDFHMSVVRA |
| | RRNDSGTYLCGAISLAPKAQIKESLRAELRVTERRAEVPTAHPSPSPRPAGQ |
| | FQTLV |
| SEQ ID NO: 262 | DIQMTQSPSSVSASVGDRVTITCRASQGISGWLAWYQQKPGKAPKFLIYAA |
| anti-nectin4 LC | STLQSGVPSRFSGSGSGTDFTLTISSLQPEDFATYYCQQANSFPPTFGGGTKV |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 263 | EVQLVESGGGLVQPGGSLRLSCAASGFTFSSYNMNWVRQAPGKGLEWVS |
| anti-nectin4 HC | YISSSSSTIYYADSVKGRFTISRDNAKNSLSLQMNSLRDEDTAVYYCARAY |
| fused to | YYGMDVWGQGTTVTVSSASTKGPSVFPLAPSSKSTSGGTAALGCLVKDYF |
| SIGLEC10 ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSMDGRFWIRVQESVMVPEGLCISVPCSFSYPRQDWTGSTPAYG |
| | YWFKAVTETTKGAPVATNHQSREVEMSTRGRFQLTGDPAKGNCSLVIRDA |
| | QMQDESQYFFRVERGSYVRYNFMNDGFFLKVTALTQKPDVYIPETLEPGQ |
| | PVTVICVFNWAFEECPPPSFSWTGAALSSQGTKPTTSHFSVLSFTPRPQDHN |
| | TDLTCHVDFSRKGVSAQRTVRLRVAYAPRDLVISISRDNTPALEPQPQGNV |
| | PYLEAQKGQFLRLLCAADSQPPATLSWVLQNRVLSSSHPWGPRPLGLELPG |
| | VKAGDSGRYTCRAENRLGSQQRALDLSVQYPPENLRVMVSQANRTVLEN |
| | LGNGTSLPVLEGQSLCLVCVTHSSPPARLSWTQRGQVLSPSQPSDPGVLELP |
| | RVQVEHEGEFTCHARHPLGSQHVSLSLSVHYSPKLLGPSCSWEAEGLHCSC |
| | SSQASPAPSLRWWLGEELLEGNSSQDSFEVTPSSAGPWANSSLSLHGGLSS |
| | GLRLRCEAWNVHGAQSGSILQLPDKKGLIST |
| SEQ ID NO: 264 | EVQLVESGGGLVQPGGSLRLSCAASGFTFSSYNMNWVRQAPGKGLEWVS |
| anti-nectin4 HC | YISSSSSTIYYADSVKGRFTISRDNAKNSLSLQMNSLRDEDTAVYYCARAY |
| fused to SIRPa | YYGMDVWGQGTTVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSEEELQVIQPDKSVLVAAGETATLRCTATSLIPVGPIQWFRGAGP |
| | GRELIYNQKEGHFPRVTTVSDLTKRNNMDFSIRIGNITPADAGTYYCVKFR |
| | KGSPDDVEFKSGAGTELSVRAKPS |
| SEQ ID NO: 265 | EVQLVESGGGLVQPGGSLRLSCAASGFTFSSYNMNWVRQAPGKGLEWVS |
| anti-nectin4 HC | YISSSSSTIYYADSVKGRFTISRDNAKNSLSLQMNSLRDEDTAVYYCARAY |
| fused to TGFbR | YYGMDVWGQGTTVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSTIPPHVQKSVNNDMIVTDNNGAVKFPQLCKFCDVRFSTCDNQ |
| | KSCMSNCSITSICEKPQEVCVAVWRKNDENITLETVCHDPKLPYHDFILEDA |
| | ASPKCIMKEKKKPGETFFMCSCSSDECNDNIIFSEEYNTSNPD |
| SEQ ID NO: 266 | EVQLVESGGGLVQPGGSLRLSCAASGFTFSSYNMNWVRQAPGKGLEWVS |
| anti-nectin4 HC | YISSSSSTIYYADSVKGRFTISRDNAKNSLSLQMNSLRDEDTAVYYCARAY |
| fused to TIM3 | YYGMDVWGQGTTVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSSEVEYRAEVGQNAYLPCFYTPAAPGNLVPVCWGKGACPVFEC |
| | GNVVLRTDERDVNYWTSRYWLNGDFRKGDVSLTIENVTLADSGIYCCRIQI |
| | PGIMNDEKFNLKLVIKPAKVTPAPTRQRDFTAAFPRMLTTRGHGPAETQTL |
| | GSLPDINLTQISTLANELRDSRLANDLRDSGATIRIG |
| SEQ ID NO: 267 | EVQLVESGGGLVQPGGSLRLSCAASGFTFSSYNMNWVRQAPGKGLEWVS |
| anti-nectin4 HC | YISSSSSTIYYADSVKGRFTISRDNAKNSLSLQMNSLRDEDTAVYYCARAY |
| fused to VEGFR | YYGMDVWGQGTTVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSSDTGRPFVEMYSEIPEIIHMTEGRELVIPCRVTSPNITVTLKKFP |
| | LDTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |
| SEQ ID NO: 268 | QVQLQQWGAGLLKPSETLSLTCAVYGGSFSGYYWSWIRQPPGKGLEWIGE |
| anti-uPAR HC | INHSGSTNYNPSLKSRVTISVDTSKNQFSLKLSSVTAADTAVYYCARGRRFG |
| fused to BTLA | DFDYWGQGTLVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSKESCDVQLYIKRQSEHSILAGDPFELECPVKYCANRPHVTWCKLN |
| | GTTCVKLEDRQTSWKEEKNISFFILHFEPVLPNDNGSYRCSANFQSNLIESHS |
| | TTLYVTDVKSASERPSKDEMAS |
| SEQ ID NO: 269 | QVQLQQWGAGLLKPSETLSLTCAVYGGSFSGYYWSWIRQPPGKGLEWIGE |
| anti-uPAR HC | INHSGSTNYNPSLKSRVTISVDTSKNQFSLKLSSVTAADTAVYYCARGRRFG |
| fused to PD1 ECD | DFDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSPGWFLDSPDRPWNPPTFSPALLVVTEGDNATFTCSFSNTSESFVLN |
| | WYRMSPSNQTDKLAAFPEDRSQPGQDCRFRVTQLPNGRDFHMSVVRARR |
| | NDSGTYLCGAISLAPKAQIKESLRAELRVTERRAEVPTAHPSPSPRPAGQFQ |
| | TLV |
| SEQ ID NO: 270 | QVQLQQWGAGLLKPSETLSLTCAVYGGSFSGYYWSWIRQPPGKGLEWIGE |
| anti-uPAR HC | INHSGSTNYNPSLKSRVTISVDTSKNQFSLKLSSVTAADTAVYYCARGRRFG |
| fused to | DFDYWGQGTLVTVSSASTKGPSVFPLAPSSKSTSGGTAALGCLVKDYFPEP |
| SIGLEC10 ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSMDGRFWIRVQESVMVPEGLCISVPCSFSYPRQDWTGSTPAYGYW |
| | FKAVTETTKGAPVATNHQSREVEMSTRGRFQLTGDPAKGNCSLVIRDAQM |
| | QDESQYFFRVERGSYVRYNFMNDGFFLKVTALTQKPDVYIPETLEPGQPVT |
| | VICVFNWAFEECPPPSFSWTGAALSSQGTKPTTSHFSVLSFTPRPQDHNTDL |
| | TCHVDFSRKGVSAQRTVRLRVAYAPRDLVISISRDNTPALEPQPQGNVPYL |
| | EAQKGQFLRLLCAADSQPPATLSWVLQNRVLSSSHPWGPRPLGLELPGVK |
| | AGDSGRYTCRAENRLGSQQRALDLSVQYPPENLRVMVSQANRTVLENLG |
| | NGTSLPVLEGQSLCLVCVTHSSPPARLSWTQRGQVLSPSQPSDPGVLELPRV |
| | QVEHEGEFTCHARHPLGSQHVSLSLSVHYSPKLLGPSCSWEAEGLHCSCSS |
| | QASPAPSLRWWLGEELLEGNSSQDSFEVTPSSAGPWANSSLSLHGGLSSGL |
| | RLRCEAWNVHGAQSGSILQLPDKKGLIST |
| SEQ ID NO: 271 | QVQLQQWGAGLLKPSETLSLTCAVYGGSFSGYYWSWIRQPPGKGLEWIGE |
| anti-uPA RHC | INHSGSTNYNPSLKSRVTISVDTSKNQFSLKLSSVTAADTAVYYCARGRRFG |
| fused to SIRPa | DFDYWGQGTLVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSEEELQVIQPDKSVLVAAGETATLRCTATSLIPVGPIQWFRGAGPGR |
| | ELIYNQKEGHFPRVTTVSDLTKRNNMDFSIRIGNITPADAGTYYCVKFRKGS |
| | PDDVEFKSGAGTELSVRAKPS |
| SEQ ID NO: 272 | QVQLQQWGAGLLKPSETLSLTCAVYGGSFSGYYWSWIRQPPGKGLEWIGE |
| anti-uPAR HC | INHSGSTNYNPSLKSRVTISVDTSKNQFSLKLSSVTAADTAVYYCARGRRFG |
| fused to TGFbR | DFDYWGQGTLVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSTIPPHVQKSVNNDMIVTDNNGAVKFPQLCKFCDVRFSTCDNQKS |
| | CMSNCSITSICEKPQEVCVAVWRKNDENITLETVCHDPKLPYHDFILEDAAS |
| | PKCIMKEKKKPGETFFMCSCSSDECNDNIIFSEEYNTSNPD |
| SEQ ID NO: 273 | QVQLQQWGAGLLKPSETLSLTCAVYGGSFSGYYWSWIRQPPGKGLEWIGE |
| anti-uPAR HC | INHSGSTNYNPSLKSRVTISVDTSKNQFSLKLSSVTAADTAVYYCARGRRFG |
| fused to TIM3 | DFDYWGQGTLVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSEVEYRAEVGQNAYLPCFYTPAAPGNLVPVCWGKGACPVFECGN |
| | VVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGS |
| | LPDINLTQISTLANELRDSRLANDLRDSGATIRIG |
| SEQ ID NO: 274 | QVQLQQWGAGLLKPSETLSLTCAVYGGSFSGYYWSWIRQPPGKGLEWIGE |
| anti-uPAR HC | INHSGSTNYNPSLKSRVTISVDTSKNQFSLKLSSVTAADTAVYYCARGRRFG |
| fused to VEGFR | DFDYWGQGTLVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSDTGRPFVEMYSEIPEIIHMTEGRELVIPCRVTSPNITVTLKKFPLDT |
| | LIPDGKRIIWDSRKGFIISNATYKEIGLLTCEATVNGHLYKTNYLTHRQTNTI |
| | IDVVLSPSHGIELSVGEKLVLNCTARTELNVGIDFNWEYPSSKHQHKKLVN |
| | RDLKTQSGSEMKKFLSTLTIDGVTRSDQGLYTCAASSGLMTKKNSTFVRVH |
| | EK |
| SEQ ID NO: 275 | QVQLVESGGGVVQPGRSLRLSCAASGFAFSRYGMHWVRQAPGKGLEWVA |
| anti-PSMA HC | VIWYDGSNKYYADSVKGRFTISRDNSKNTQYLQMNSLRAEDTAVYYCAR |
| fused to BTLA | GGDFLYYYYYGMDVWGQGTTVTVSSASTKGPSVFPLAPSSKSTSGGTAAL |
| ECD | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSKESCDVQLYIKRQSEHSILAGDPFELECPVKYCAN |
| | RPHVTWCKLNGTTCVKLEDRQTSWKEEKNISFFILHFEPVLPNDNGSYRCS |
| | ANFQSNLIESHSTTLYVTDVKSASERPSKDEMAS |
| SEQ ID NO: 276 | QVQLVESGGGVVQPGRSLRLSCAASGFAFSRYGMHWVRQAPGKGLEWVA |
| anti-PSMA HC | VIWYDGSNKYYADSVKGRFTISRDNSKNTQYLQMNSLRAEDTAVYYCAR |
| fused to PD1 ECD | GGDFLYYYYYGMDVWGQGTTVTVSSASTKGPSVFPLAPSSKSTSGGTAAL |
| | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSPGWFLDSPDRPWNPPTFSPALLVVTEGDNATFTC |
| | SFSNTSESFVLNWYRMSPSNQTDKLAAFPEDRSQPGQDCRFRVTQLPNGRD |
| | FHMSVVRARRNDSGTYLCGAISLAPKAQIKESLRAELRVTERRAEVPTAHP |
| | SPSPRPAGQFQTLV |
| SEQ ID NO: 277 | QVQLVESGGGVVQPGRSLRLSCAASGFAFSRYGMHWVRQAPGKGLEWVA |
| anti-PSMA HC | VIWYDGSNKYYADSVKGRFTISRDNSKNTQYLQMNSLRAEDTAVYYCAR |
| fused to | GGDFLYYYYYGMDVWGQGTTVTVSSASTKGPSVFPLAPSSKSTSGGTAAL |
| SIGLEC10 ECD | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSMDGRFWIRVQESVMVPEGLCISVPCSFSYPRQDW |
| | TGSTPAYGYWFKAVTETTKGAPVATNHQSREVEMSTRGRFQLTGDPAKG |
| | NCSLVIRDAQMQDESQYFFRVERGSYVRYNFMNDGFFLKVTALTQKPDVY |
| | IPETLEPGQPVTVICVFNWAFEECPPPSFSWTGAALSSQGTKPTTSHFSVLSF |
| | TPRPQDHNTDLTCHVDFSRKGVSAQRTVRLRVAYAPRDLVISISRDNTPAL |
| | EPQPQGNVPYLEAQKGQFLRLLCAADSQPPATLSWVLQNRVLSSSHPWGP |
| | RPLGLELPGVKAGDSGRYTCRAENRLGSQQRALDLSVQYPPENLRVMVSQ |
| | ANRTVLENLGNGTSLPVLEGQSLCLVCVTHSSPPARLSWTQRGQVLSPSQP |
| | SDPGVLELPRVQVEHEGEFTCHARHPLGSQHVSLSLSVHYSPKLLGPSCSW |
| | EAEGLHCSCSSQASPAPSLRWWLGEELLEGNSSQDSFEVTPSSAGPWANSS |
| | LSLHGGLSSGLRLRCEAWNVHGAQSGSILQLPDKKGLIST |
| SEQ ID NO: 278 | QVQLVESGGGVVQPGRSLRLSCAASGFAFSRYGMHWVRQAPGKGLEWVA |
| anti-PSMA HC | VIWYDGSNKYYADSVKGRFTISRDNSKNTQYLQMNSLRAEDTAVYYCAR |
| fused to SIRPa | GGDFLYYYYYGMDVWGQGTTVTVSSASTKGPSVFPLAPSSKSTSGGTAAL |
| ECD | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSEEELQVIQPDKSVLVAAGETATLRCTATSLIPVGPI |
| | QWFRGAGPGRELIYNQKEGHFPRVTTVSDLTKRNNMDFSIRIGNITPADAG |
| | TYYCVKFRKGSPDDVEFKSGAGTELSVRAKPS |
| SEQ ID NO: 279 | QVQLVESGGGVVQPGRSLRLSCAASGFAFSRYGMHWVRQAPGKGLEWVA |
| anti-PSMA HC | VIWYDGSNKYYADSVKGRFTISRDNSKNTQYLQMNSLRAEDTAVYYCAR |
| fused to TGFbR | GGDFLYYYYYGMDVWGQGTTVTVSSASTKGPSVFPLAPSSKSTSGGTAAL |
| ECD | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSTIPPHVQKSVNNDMIVTDNNGAVKFPQLCKFCDV |
| | RFSTCDNQKSCMSNCSITSICEKPQEVCVAVWRKNDENITLETVCHDPKLP |
| | YHDFILEDAASPKCIMKEKKKPGETFFMCSCSSDECNDNIIFSEEYNTSNPD |
| SEQ ID NO: 280 | QVQLVESGGGVVQPGRSLRLSCAASGFAFSRYGMHWVRQAPGKGLEWVA |
| anti-PSMA HC | VIWYDGSNKYYADSVKGRFTISRDNSKNTQYLQMNSLRAEDTAVYYCAR |
| fused to TIM3 | GGDFLYYYYYGMDVWGQGTTVTVSSASTKGPSVFPLAPSSKSTSGGTAAL |
| ECD | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSSEVEYRAEVGQNAYLPCFYTPAAPGNLVPVCWG |
| | KGACPVFECGNVVLRTDERDVNYWTSRYWLNGDFRKGDVSLTIENVTLA |
| | DSGIYCCRIQIPGIMNDEKFNLKLVIKPAKVTPAPTRQRDFTAAFPRMLTTR |
| | GHGPAETQTLGSLPDINLTQISTLANELRDSRLANDLRDSGATIRIG |
| SEQ ID NO: 281 | QVQLVESGGGVVQPGRSLRLSCAASGFAFSRYGMHWVRQAPGKGLEWVA |
| anti-PSMA HC | VIWYDGSNKYYADSVKGRFTISRDNSKNTQYLQMNSLRAEDTAVYYCAR |
| fused to VEGFR | GGDFLYYYYYGMDVWGQGTTVTVSSASTKGPSVFPLAPSSKSTSGGTAAL |
| ECD | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSSDTGRPFVEMYSEIPEIIHMTEGRELVIPCRVTSPNI |
| | TVTLKKFPLDTLIPDGKRIIWDSRKGFIISNATYKEIGLLTCEATVNGHLYKT |
| | NYLTHRQTNTIIDVVLSPSHGIELSVGEKLVLNCTARTELNVGIDFNWEYPS |
| | SKHQHKKLVNRDLKTQSGSEMKKFLSTLTIDGVTRSDQGLYTCAASSGLM |
| | TKKNSTFVRVHEK |
| SEQ ID NO: 282 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| HC fused to | PWFAYWGQGTPVTVSSASTKGPSVFPLAPSSKSTSGGTAALGCLVKDYFPE |
| BTLAECD | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSKESCDVQLYIKRQSEHSILAGDPFELECPVKYCANRPHVTWCKLN |
| | GTTCVKLEDRQTSWKEEKNISFFILHFEPVLPNDNGSYRCSANFQSNLIESHS |
| | TTLYVTDVKSASERPSKDEMAS |
| SEQ ID NO: 283 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| HC fused to PD1 | PWFAYWGQGTPVTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSPGWFLDSPDRPWNPPTFSPALLVVTEGDNATFTCSFSNTSESFVLN |
| | WYRMSPSNQTDKLAAFPEDRSQPGQDCRFRVTQLPNGRDFHMSVVRARR |
| | NDSGTYLCGAISLAPKAQIKESLRAELRVTERRAEVPTAHPSPSPRPAGQFQ |
| | TLV |
| SEQ ID NO: 284 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| HC fused to | PWFAYWGQGTPVTVSSASTKGPSVFPLAPSSKSTSGGTAALGCLVKDYFPE |
| SIGLEC10 ECD | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSMDGRFWIRVQESVMVPEGLCISVPCSFSYPRQDWTGSTPAYGYW |
| | FKAVTETTKGAPVATNHQSREVEMSTRGRFQLTGDPAKGNCSLVIRDAQM |
| | QDESQYFFRVERGSYVRYNFMNDGFFLKVTALTQKPDVYIPETLEPGQPVT |
| | VICVFNWAFEECPPPSFSWTGAALSSQGTKPTTSHFSVLSFTPRPQDHNTDL |
| | TCHVDFSRKGVSAQRTVRLRVAYAPRDLVISISRDNTPALEPQPQGNVPYL |
| | EAQKGQFLRLLCAADSQPPATLSWVLQNRVLSSSHPWGPRPLGLELPGVK |
| | AGDSGRYTCRAENRLGSQQRALDLSVQYPPENLRVMVSQANRTVLENLG |
| | NGTSLPVLEGQSLCLVCVTHSSPPARLSWTQRGQVLSPSQPSDPGVLELPRV |
| | QVEHEGEFTCHARHPLGSQHVSLSLSVHYSPKLLGPSCSWEAEGLHCSCSS |
| | QASPAPSLRWWLGEELLEGNSSQDSFEVTPSSAGPWANSSLSLHGGLSSGL |
| | RLRCEAWNVHGAQSGSILQLPDKKGLIST |
| SEQ ID NO: 285 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| HC fused to | PWFAYWGQGTPVTVSSASTKGPSVFPLAPSSKSTSGGTAALGCLVKDYFPE |
| SIRPa ECD | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSEEELQVIQPDKSVLVAAGETATLRCTATSLIPVGPIQWFRGAGPGR |
| | ELIYNQKEGHFPRVTTVSDLTKRNNMDFSIRIGNITPADAGTYYCVKFRKGS |
| | PDDVEFKSGAGTELSVRAKPS |
| SEQ ID NO: 286 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| HC fused to | PWFAYWGQGTPVTVSSASTKGPSVFPLAPSSKSTSGGTAALGCLVKDYFPE |
| TGFbR ECD | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSTIPPHVQKSVNNDMIVTDNNGAVKFPQLCKFCDVRFSTCDNQKS |
| | CMSNCSITSICEKPQEVCVAVWRKNDENITLETVCHDPKLPYHDFILEDAAS |
| | PKCIMKEKKKPGETFFMCSCSSDECNDNIIFSEEYNTSNPD |
| SEQ ID NO: 287 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| HC fused to TIM3 | PWFAYWGQGTPVTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSEVEYRAEVGQNAYLPCFYTPAAPGNLVPVCWGKGACPVFECGN |
| | VVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGS |
| | LPDINLTQISTLANELRDSRLANDLRDSGATIRIG |
| SEQ ID NO: 288 | EVQLVESGGGVVQPGRSLRLSCSASGFDFTTYWMSWVRQAPGKGLEWIGE |
| anti-CEACAM5 | IHPDSSTINYAPSLKDRFTISRDNAKNTLFLQMDSLRPEDTGVYFCASLYFGF |
| HC fused to | PWFAYWGQGTPVTVSSASTKGPSVFPLAPSSKSTSGGTAALGCLVKDYFPE |
| VEGFR ECD | PVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSDTGRPFVEMYSEIPEIIHMTEGRELVIPCRVTSPNITVTLKKFPLDT |
| | LIPDGKRIIWDSRKGFIISNATYKEIGLLTCEATVNGHLYKTNYLTHRQTNTI |
| | IDVVLSPSHGIELSVGEKLVLNCTARTELNVGIDFNWEYPSSKHQHKKLVN |
| | RDLKTQSGSEMKKFLSTLTIDGVTRSDQGLYTCAASSGLMTKKNSTFVRVH |
| | EK |
| SEQ ID NO: 289 | EVQLLESGGGLVQPGGSLRLSCAVSGFTFNSFAMSWVRQAPGKGLEWVSA |
| anti-CD38 HC | ISGSGGGTYYADSVKGRFTISRDNSKNTLYLQMNSLRAEDTAVYFCAKDKI |
| fused to BTLA | LWFGEPVFDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSKESCDVQLYIKRQSEHSILAGDPFELECPVKYCANRPHVT |
| | WCKLNGTTCVKLEDRQTSWKEEKNISFFILHFEPVLPNDNGSYRCSANFQS |
| | NLIESHSTTLYVTDVKSASERPSKDEMAS |
| SEQ ID NO: 290 | EIVLTQSPATLSLSPGERATLSCRASQSVSSYLAWYQQKPGQAPRLLIYDAS |
| anti-CD38 LC | NRATGIPARFSGSGSGTDFTLTISSLEPEDFAVYYCQQRSNWPPTFGQGTKV |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 291 | EIVLTQSPATLSLSPGERATLSCRASQSVSSYLAWYQQKPGQAPRLLIYDAS |
| anti-CD38 LC | NRATGIPARFSGSGSGTDFTLTISSLEPEDFAVYYCQQRSNWPPTFGQGTKV |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 292 | EIVLTQSPATLSLSPGERATLSCRASQSVSSYLAWYQQKPGQAPRLLIYDAS |
| anti- CD38 LC | NRATGIPARFSGSGSGTDFTLTISSLEPEDFAVYYCQQRSNWPPTFGQGTKV |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 293 | EIVLTQSPATLSLSPGERATLSCRASQSVSSYLAWYQQKPGQAPRLLIYDAS |
| anti-CD38 LC | NRATGIPARFSGSGSGTDFTLTISSLEPEDFAVYYCQQRSNWPPTFGQGTKV |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 294 | EVQLLESGGGLVQPGGSLRLSCAVSGFTFNSFAMSWVRQAPGKGLEWVSA |
| anti-CD38 HC | ISGSGGGTYYADSVKGRFTISRDNSKNTLYLQMNSLRAEDTAVYFCAKDKI |
| fused to PD1 ECD | LWFGEPVFDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSPGWFLDSPDRPWNPPTFSPALLVVTEGDNATFTCSFSNTS |
| | ESFVLNWYRMSPSNQTDKLAAFPEDRSQPGQDCRFRVTQLPNGRDFHMSV |
| | VRARRNDSGTYLCGAISLAPKAQIKESLRAELRVTERRAEVPTAHPSPSPRP |
| | AGQFQTLV |
| SEQ ID NO: 295 | EIVLTQSPATLSLSPGERATLSCRASQSVSSYLAWYQQKPGQAPRLLIYDAS |
| anti- CD38 LC | NRATGIPARFSGSGSGTDFTLTISSLEPEDFAVYYCQQRSNWPPTFGQGTKV |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 296 | EVQLLESGGGLVQPGGSLRLSCAVSGFTFNSFAMSWVRQAPGKGLEWVSA |
| anti-CD38 HC | ISGSGGGTYYADSVKGRFTISRDNSKNTLYLQMNSLRAEDTAVYFCAKDKI |
| fused to | LWFGEPVFDYWGQGTLVTVSSASTKGPSVFPLAPSSKSTSGGTAALGCLVK |
| SIGLEC10 ECD | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSMDGRFWIRVQESVMVPEGLCISVPCSFSYPRQDWTGSTP |
| | AYGYWFKAVTETTKGAPVATNHQSREVEMSTRGRFQLTGDPAKGNCSLVI |
| | RDAQMQDESQYFFRVERGSYVRYNFMNDGFFLKVTALTQKPDVYIPETLE |
| | PGQPVTVICVFNWAFEECPPPSFSWTGAALSSQGTKPTTSHFSVLSFTPRPQ |
| | DHNTDLTCHVDFSRKGVSAQRTVRLRVAYAPRDLVISISRDNTPALEPQPQ |
| | GNVPYLEAQKGQFLRLLCAADSQPPATLSWVLQNRVLSSSHPWGPRPLGL |
| | ELPGVKAGDSGRYTCRAENRLGSQQRALDLSVQYPPENLRVMVSQANRTV |
| | LENLGNGTSLPVLEGQSLCLVCVTHSSPPARLSWTQRGQVLSPSQPSDPGV |
| | LELPRVQVEHEGEFTCHARHPLGSQHVSLSLSVHYSPKLLGPSCSWEAEGL |
| | HCSCSSQASPAPSLRWWLGEELLEGNSSQDSFEVTPSSAGPWANSSLSLHG |
| | GLSSGLRLRCEAWNVHGAQSGSILQLPDKKGLIST |
| SEQ ID NO: 297 | EVQLLESGGGLVQPGGSLRLSCAVSGFTFNSFAMSWVRQAPGKGLEWVSA |
| anti-CD38 HC | ISGSGGGTYYADSVKGRFTISRDNSKNTLYLQMNSLRAEDTAVYFCAKDKI |
| fused to SIRPa | LWFGEPVFDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSEEELQVIQPDKSVLVAAGETATLRCTATSLIPVGPIQWFRG |
| | AGPGRELIYNQKEGHFPRVTTVSDLTKRNNMDFSIRIGNITPADAGTYYCV |
| | KFRKGSPDDVEFKSGAGTELSVRAKPS |
| SEQ ID NO: 298 | EVQLLESGGGLVQPGGSLRLSCAVSGFTFNSFAMSWVRQAPGKGLEWVSA |
| anti-CD38 HC | ISGSGGGTYYADSVKGRFTISRDNSKNTLYLQMNSLRAEDTAVYFCAKDKI |
| fused to TGFbR | LWFGEPVFDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSTIPPHVQKSVNNDMIVTDNNGAVKFPQLCKFCDVRFSTC |
| | DNQKSCMSNCSITSICEKPQEVCVAVWRKNDENITLETVCHDPKLPYHDFIL |
| | EDAASPKCIMKEKKKPGETFFMCSCSSDECNDNIIFSEEYNTSNPD |
| SEQ ID NO: 299 | EVQLLESGGGLVQPGGSLRLSCAVSGFTFNSFAMSWVRQAPGKGLEWVSA |
| anti-CD38 HC | ISGSGGGTYYADSVKGRFTISRDNSKNTLYLQMNSLRAEDTAVYFCAKDKI |
| fused to TIM3 | LWFGEPVFDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSSEVEYRAEVGQNAYLPCFYTPAAPGNLVPVCWGKGACP |
| | VFECGNVVLRTDERDVNYWTSRYWLNGDFRKGDVSLTIENVTLADSGIYC |
| | CRIQIPGIMNDEKFNLKLVIKPAKVTPAPTRQRDFTAAFPRMLTTRGHGPAE |
| | TQTLGSLPDINLTQISTLANELRDSRLANDLRDSGATIRIG |
| SEQ ID NO: 300 | EVQLLESGGGLVQPGGSLRLSCAVSGFTFNSFAMSWVRQAPGKGLEWVSA |
| anti-CD38 HC | ISGSGGGTYYADSVKGRFTISRDNSKNTLYLQMNSLRAEDTAVYFCAKDKI |
| fused to VEGFR | LWFGEPVFDYWGQGTLVTVSSASTKGPSVFPLAPSSKSTSGGTAALGCLVK |
| ECD | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSSDTGRPFVEMYSEIPEIIHMTEGRELVIPCRVTSPNITVTLK |
| | KFPLDTLIPDGKRIIWDSRKGFIISNATYKEIGLLTCEATVNGHLYKTNYLTH |
| | RQTNTIIDVVLSPSHGIELSVGEKLVLNCTARTELNVGIDFNWEYPSSKHQH |
| | KKLVNRDLKTQSGSEMKKFLSTLTIDGVTRSDQGLYTCAASSGLMTKKNS |
| | TFVRVHEK |
| SEQ ID NO: 301 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| HC fused to | NYWYFDVWGQGTLVTVSSASTKGPSVFPLAPSSKSTSGGTAALGCLVKDY |
| BTLA ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSKESCDVQLYIKRQSEHSILAGDPFELECPVKYCANRPHVTWC |
| | KLNGTTCVKLEDRQTSWKEEKNISFFILHEEPVLPNDNGSYRCSANFQSNLI |
| | ESHSTTLYVTDVKSASERPSKDEMAS |
| SEQ ID NO: 302 | DIQMTQSPSSLSASVGDRVTITCKASQDVGIAVAWYQQKPGKVPKLLIYWA |
| anti-SLAMF7 LC | STRHTGVPDRFSGSGSGTDFTLTISSLQPEDVATYYCQQYSSYPYTFGQGTK |
| fused to PD1 ECD | VEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPV |
| | TKSFNRGECGGGGSGGGGSGGGGSPGWFLDSPDRPWNPPTFSPALLVVTE |
| | GDNATFTCSFSNTSESFVLNWYRMSPSNQTDKLAAFPEDRSQPGQDCRFRV |
| | TQLPNGRDFHMSVVRARRNDSGTYLCGAISLAPKAQIKESLRAELRVTERR |
| | AEVPTAHPSPSPRPAGQFQTLV |
| SEQ ID NO: 303 | DIQMTQSPSSLSASVGDRVTITCKASQDVGIAVAWYQQKPGKVPKLLIYWA |
| anti-SLAMF7 LC | STRHTGVPDRFSGSGSGTDFTLTISSLQPEDVATYYCQQYSSYPYTFGQGTK |
| fused to | VEIKRTVAAPSVFIFPPSDEQLKSGTASVVCLLNNFYPREAKVQWKVDNAL |
| SIGLEC10 ECD | QSGNSQESVTEQDSKDSTYSLSSTLTLSKADYEKHKVYACEVTHQGLSSPV |
| | TKSFNRGECGGGGSGGGGSGGGGSMDGRFWIRVQESVMVPEGLCISVPCS |
| | FSYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 304 | DIQMTQSPSSLSASVGDRVTITCKASQDVGIAVAWYQQKPGKVPKLLIYWA |
| anti-SLAMF7 LC | STRHTGVPDRFSGSGSGTDFTLTISSLQPEDVATYYCQQYSSYPYTFGQGTK |
| fused to SIRPa | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSEEELQVIQPDKSVLVAAGETATLRCT |
| | ATSLIPVGPIQWFRGAGPGRELIYNQKEGHFPRVTTVSDLTKRNNMDFSIRI |
| | GNITPADAGTYYCVKFRKGSPDDVEFKSGAGTELSVRAKPS |
| SEQ ID NO: 305 | DIQMTQSPSSLSASVGDRVTITCKASQDVGIAVAWYQQKPGKVPKLLIYWA |
| anti-SLAMF7 LC | STRHTGVPDRFSGSGSGTDFTLTISSLQPEDVATYYCQQYSSYPYTFGQGTK |
| fused to TIM3 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSSEVEYRAEVGQNAYLPCFYTPAAPGN |
| | LVPVCWGKGACPVFECGNVVLRTDERDVNYWTSRYWLNGDFRKGDVSL |
| | TIENVTLADSGIYCCRIQIPGIMNDEKFNLKLVIKPAKVTPAPTRQRDFTAAF |
| | PRMLTTRGHGPAETQTLGSLPDINLTQISTLANELRDSRLANDLRDSGATIRI |
| | G |
| SEQ ID NO: 306 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| HC fused to PD1 | NYWYFDVWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSPGWFLDSPDRPWNPPTFSPALLVVTEGDNATFTCSFSNTSESF |
| | VLNWYRMSPSNQTDKLAAFPEDRSQPGQDCRFRVTQLPNGRDFHMSVVR |
| | ARRNDSGTYLCGAISLAPKAQIKESLRAELRVTERRAEVPTAHPSPSPRPAG |
| | QFQTLV |
| SEQ ID NO: 307 | DIQMTQSPSSLSASVGDRVTITCKASQDVGIAVAWYQQKPGKVPKLLIYWA |
| anti-SLAMF7 LC | STRHTGVPDRFSGSGSGTDFTLTISSLQPEDVATYYCQQYSSYPYTFGQGTK |
| fused to BTLA | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSKESCDVQLYIKRQSEHSILAGDPFELE |
| | CPVKYCANRPHVTWCKLNGTTCVKLEDRQTSWKEEKNISFFILHFEPVLPN |
| | DNGSYRCSANFQSNLIESHSTTLYVTDVKSASERPSKDEMAS |
| SEQ ID NO: 308 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| HC fused to | NYWYFDVWGQGTLVTVSSASTKGPSVFPLAPSSKSTSGGTAALGCLVKDY |
| SIGLEC10 ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSMDGRFWIRVQESVMVPEGLCISVPCSFSYPRQDWTGSTPAY |
| | GYWFKAVTETTKGAPVATNHQSREVEMSTRGRFQLTGDPAKGNCSLVIRD |
| | AQMQDESQYFFRVERGSYVRYNFMNDGFFLKVTALTQKPDVYIPETLEPG |
| | QPVTVICVFNWAFEECPPPSFSWTGAALSSQGTKPTTSHFSVLSFTPRPQDH |
| | NTDLTCHVDFSRKGVSAQRTVRLRVAYAPRDLVISISRDNTPALEPQPQGN |
| | VPYLEAQKGQFLRLLCAADSQPPATLSWVLQNRVLSSSHPWGPRPLGLELP |
| | GVKAGDSGRYTCRAENRLGSQQRALDLSVQYPPENLRVMVSQANRTVLE |
| | NLGNGTSLPVLEGQSLCLVCVTHSSPPARLSWTQRGQVLSPSQPSDPGVLE |
| | LPRVQVEHEGEFTCHARHPLGSQHVSLSLSVHYSPKLLGPSCSWEAEGLHC |
| | SCSSQASPAPSLRWWLGEELLEGNSSQDSFEVTPSSAGPWANSSLSLHGGLS |
| | SGLRLRCEAWNVHGAQSGSILQLPDKKGLIST |
| SEQ ID NO: 309 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| HC fused to | NYWYFDVWGQGTLVTVSSASTKGPSVFPLAPSSKSTSGGTAALGCLVKDY |
| SIRPa EC | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSEEELQVIQPDKSVLVAAGETATLRCTATSLIPVGPIQWFRGA |
| | GPGRELIYNQKEGHFPRVTTVSDLTKRNNMDFSIRIGNITPADAGTYYCVKF |
| | RKGSPDDVEFKSGAGTELSVRAKPS |
| SEQ ID NO: 310 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| HC fused to | NYWYFDVWGQGTLVTVSSASTKGPSVFPLAPSSKSTSGGTAALGCLVKDY |
| TGFbR EC | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSTIPPHVQKSVNNDMIVTDNNGAVKFPQLCKFCDVRFSTCDN |
| | QKSCMSNCSITSICEKPQEVCVAVWRKNDENITLETVCHDPKLPYHDFILED |
| | AASPKCIMKEKKKPGETFFMCSCSSDECNDNIIFSEEYNTSNPD |
| SEQ ID NO: 311 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| HC fused to TIM3 | NYWYFDVWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSSEVEYRAEVGQNAYLPCFYTPAAPGNLVPVCWGKGACPVF |
| | ECGNVVLRTDERDVNYWTSRYWLNGDFRKGDVSLTIENVTLADSGIYCCR |
| | IQIPGIMNDEKFNLKLVIKPAKVTPAPTRQRDFTAAFPRMLFERGHGPAETQ |
| | TLGSLPDINLTQISTLANELRDSRLANDLRDSGATIRIG |
| SEQ ID NO: 312 | EVQLVESGGGLVQPGGSLRLSCAASGFDFSRYWMSWVRQAPGKGLEWIG |
| anti-SLAMF7 | EINPDSSTINYAPSLKDKFIISRDNAKNSLYLQMNSLRAEDTAVYYCARPDG |
| HC fused to | NYWYFDVWGQGTLVTVSSASTKGPSVFPLAPSSKSTSGGTAALGCLVKDY |
| VEGFR ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSSDTGRPFVEMYSEIPEIIHMTEGRELVIPCRVTSPNITVTLKKF |
| | PLDTLIPDGKRIIWDSRKGFIISNATYKEIGLLTCEATVNGHLYKTNYLTHRQ |
| | TNTIIDVVLSPSHGIELSVGEKLVLNCTARTELNVGIDFNWEYPSSKHQHKK |
| | LVNRDLKTQSGSEMKKFLSTLTIDGVTRSDQGLYTCAASSGLMTKKNSTFV |
| | RVHEK |
| SEQ ID NO: 313 | QVQLQESGPGLVRPSQTLSLTCTVSGYSITSDHAWSWVRQPPGRGLEWIGY |
| anti-IL6R HC | ISYSGITTYNPSLKSRVTMLRDTSKNQFSLRLSSVTAADTAVYYCARSLART |
| fused to BTLA | TAMDYWGQGSLVTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSKESCDVQLYIKRQSEHSILAGDPFELECPVKYCANRPHVTWCKLN |
| | GTTCVKLEDRQTSWKEEKNISFFILHFEPVLPNDNGSYRCSANFQSNLIESHS |
| | TTLYVTDVKSASERPSKDEMAS |
| SEQ ID NO: 314 | DIQMTQSPSSLSASVGDRVTITCRASQDISSYLNWYQQKPGKAPKLLIYYTS |
| anti-IL6R LC | RLHSGVPSRFSGSGSGTDFTFTISSLQPEDIATYYCQQGNTLPYTFGQGTKV |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 315 | DIQMTQSPSSLSASVGDRVTITCRASQDISSYLNWYQQKPGKAPKLLIYYTS |
| anti-IL6R LC | RLHSGVPSRFSGSGSGTDFTFTISSLQPEDIATYYCQQGNTLPYTFGQGTKV |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 316 | DIQMTQSPSSLSASVGDRVTITCRASQDISSYLNWYQQKPGKAPKLLIYYTS |
| anti-IL6R LC | RLHSGVPSRFSGSGSGTDFTFTISSLQPEDIATYYCQQGNTLPYTFGQGTKV |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 317 | DIQMTQSPSSLSASVGDRVTITCRASQDISSYLNWYQQKPGKAPKLLIYYTS |
| anti-IL6R LC | RLHSGVPSRFSGSGSGTDFTFTISSLQPEDIATYYCQQGNTLPYTFGQGTKV |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 318 | QVQLQESGPGLVRPSQTLSLTCTVSGYSITSDHAWSWVRQPPGRGLEWIGY |
| anti-IL6R HC | ISYSGITTYNPSLKSRVTMLRDTSKNQFSLRLSSVTAADTAVYYCARSLART |
| fused to PD1 ECD | TAMDYWGQGSLVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSPGWFLDSPDRPWNPPTFSPALLVVTEGDNATFTCSFSNTSESFVLN |
| | WYRMSPSNQTDKLAAFPEDRSQPGQDCRFRVTQLPNGRDFHMSVVRARR |
| | NDSGTYLCGAISLAPKAQIKESLRAELRVTERRAEVPTAHPSPSPRPAGQFQ |
| | TLV |
| SEQ ID NO: 319 | DIQMTQSPSSLSASVGDRVTITCRASQDISSYLNWYQQKPGKAPKLLIYYTS |
| anti-IL6R LC | RLHSGVPSRFSGSGSGTDFTFTISSLQPEDIATYYCQQGNTLPYTFGQGTKV |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 320 | QVQLQESGPGLVRPSQTLSLTCTVSGYSITSDHAWSWVRQPPGRGLEWIGY |
| anti-IL6R HC | ISYSGITTYNPSLKSRVTMLRDTSKNQFSLRLSSVTAADTAVYYCARSLART |
| fused to | TAMDYWGQGSLVTVSSASTKGPSVFPLAPSSKSTSGGTAALGCLVKDYFPE |
| SIGLEC10 ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSMDGRFWIRVQESVMVPEGLCISVPCSFSYPRQDWTGSTPAYGYW |
| | FKAVTETTKGAPVATNHQSREVEMSTRGRFQLTGDPAKGNCSLVIRDAQM |
| | QDESQYFFRVERGSYVRYNFMNDGFFLKVTALTQKPDVYIPETLEPGQPVT |
| | VICVFNWAFEECPPPSFSWTGAALSSQGTKPTTSHFSVLSFTPRPQDHNTDL |
| | TCHVDFSRKGVSAQRTVRLRVAYAPRDLVISISRDNTPALEPQPQGNVPYL |
| | EAQKGQFLRLLCAADSQPPATLSWVLQNRVLSSSHPWGPRPLGLELPGVK |
| | AGDSGRYTCRAENRLGSQQRALDLSVQYPPENLRVMVSQANRTVLENLG |
| | NGTSLPVLEGQSLCLVCVTHSSPPARLSWTQRGQVLSPSQPSDPGVLELPRV |
| | QVEHEGEFTCHARHPLGSQHVSLSLSVHYSPKLLGPSCSWEAEGLHCSCSS |
| | QASPAPSLRWWLGEELLEGNSSQDSFEVTPSSAGPWANSSLSLHGGLSSGL |
| | RLRCEAWNVHGAQSGSILQLPDKKGLIST |
| SEQ ID NO: 321 | QVQLQESGPGLVRPSQTLSLTCTVSGYSITSDHAWSWVRQPPGRGLEWIGY |
| anti-IL6R HC | ISYSGITTYNPSLKSRVTMLRDTSKNQFSLRLSSVTAADTAVYYCARSLART |
| fused to SIRPa | TAMDYWGQGSLVTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSEEELQVIQPDKSVLVAAGETATLRCTATSLIPVGPIQWFRGAGPGR |
| | ELIYNQKEGHFPRVTTVSDLTKRNNMDFSIRIGNITPADAGTYYCVKFRKGS |
| | PDDVEFKSGAGTELSVRAKPS |
| SEQ ID NO: 322 | QVQLQESGPGLVRPSQTLSLTCTVSGYSITSDHAWSWVRQPPGRGLEWIGY |
| anti-IL6R HC | ISYSGITTYNPSLKSRVTMLRDTSKNQFSLRLSSVTAADTAVYYCARSLART |
| fused to TGFbR | TAMDYWGQGSLVTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSTIPPHVQKSVNNDMIVTDNNGAVKFPQLCKFCDVRFSTCDNQKS |
| | CMSNCSITSICEKPQEVCVAVWRKNDENITLETVCHDPKLPYHDFILEDAAS |
| | PKCIMKEKKKPGETFFMCSCSSDECNDNIIFSEEYNTSNPD |
| SEQ ID NO: 323 | QVQLQESGPGLVRPSQTLSLTCTVSGYSITSDHAWSWVRQPPGRGLEWIGY |
| anti-IL6R HC | ISYSGITTYNPSLKSRVTMLRDTSKNQFSLRLSSVTAADTAVYYCARSLART |
| fused to TIM3 | TAMDYWGQGSLVTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSEVEYRAEVGQNAYLPCFYTPAAPGNLVPVCWGKGACPVFECGN |
| | VVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGS |
| | LPDINLTQISTLANELRDSRLANDLRDSGATIRIG |
| SEQ ID NO: 324 | QVQLQESGPGLVRPSQTLSLTCTVSGYSITSDHAWSWVRQPPGRGLEWIGY |
| anti-IL6R HC | ISYSGITTYNPSLKSRVTMLRDTSKNQFSLRLSSVTAADTAVYYCARSLART |
| fused to VEGFR | TAMDYWGQGSLVTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSDTGRPFVEMYSEIPEIIHMTEGRELVIPCRVTSPNITVTLKKFPLDT |
| | LIPDGKRIIWDSRKGFIISNATYKEIGLLTCEATVNGHLYKTNYLTHRQTNTI |
| | IDVVLSPSHGIELSVGEKLVLNCTARTELNVGIDFNWEYPSSKHQHKKLVN |
| | RDLKTQSGSEMKKFLSTLTIDGVTRSDQGLYTCAASSGLMTKKNSTFVRVH |
| | EK |
| SEQ ID NO: 325 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to BTLA | QLYFDYWGQGTLVTVSSASTKGPSVFPLAPCSRSTSESTAALGCLVKDYFP |
| ECD | EPVTVSWNSGALTSGVHTFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSKESCDVQLYIKRQSEHSILAGDPFELECPVKYCANRPHVTWCKLNGT |
| | TCVKLEDRQTSWKEEKNISFFILHFEPVLPNDNGSYRCSANFQSNLIESHSTT |
| | LYVTDVKSASERPSKDEMAS |
| SEQ ID NO: 326 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to PD1 ECD | VEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPV |
| | TKSFNRGECGGGGSGGGGSGGGGSPGWFLDSPDRPWNPPTFSPALLVVTE |
| | GDNATFTCSFSNTSESFVLNWYRMSPSNQTDKLAAFPEDRSQPGQDCRFRV |
| | TQLPNGRDFHMSVVRARRNDSGTYLCGAISLAPKAQIKESLRAELRVTERR |
| | AEVPTAHPSPSPRPAGQFQTLV |
| SEQ ID NO: 327 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to | VEIKRTVAAPSVFIFPPSDEQLKSGTASVVCLLNNFYPREAKVQWKVDNAL |
| SIGLEC10 ECD | QSGNSQESVTEQDSKDSTYSLSSTLTLSKADYEKHKVYACEVTHQGLSSPV |
| | TKSFNRGECGGGGSGGGGSGGGGSMDGRFWIRVQESVMVPEGLCISVPCS |
| | FSYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 328 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to SIRPa | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSEEELQVIQPDKSVLVAAGETATLRCT |
| | ATSLIPVGPIQWFRGAGPGRELIYNQKEGHFPRVTTVSDLTKRNNMDFSIRI |
| | GNITPADAGTYYCVKFRKGSPDDVEFKSGAGTELSVRAKPS |
| SEQ ID NO: 329 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to TIM3 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSSEVEYRAEVGQNAYLPCFYTPAAPGN |
| | LVPVCWGKGACPVFECGNVVLRTDERDVNYWTSRYWLNGDFRKGDVSL |
| | TIENVTLADSGIYCCRIQIPGIMNDEKFNLKLVIKPAKVTPAPTRQRDFTAAF |
| | PRMLTTRGHGPAETQTLGSLPDINLTQISTLANELRDSRLANDLRDSGATIRI |
| | G |
| SEQ ID NO: 330 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to PD1 ECD | QLYFDYWGQGTLVTVSSASTKGPSVFPLAPCSRSTSESTAALGCLVKDYFP |
| | EPVTVSWNSGALTSGVHTFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSPGWFLDSPDRPWNPPTFSPALLVVTEGDNATFTCSFSNTSESFVLNW |
| | YRMSPSNQTDKLAAFPEDRSQPGQDCRFRVTQLPNGRDFHMSVVRARRND |
| | SGTYLCGAISLAPKAQIKESLRAELRVTERRAEVPTAHPSPSPRPAGQFQTL |
| | V |
| SEQ ID NO: 331 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to BTLA | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSKESCDVQLYIKRQSEHSILAGDPFELE |
| | CPVKYCANRPHVTWCKLNGTTCVKLEDRQTSWKEEKNISFFILHFEPVLPN |
| | DNGSYRCSANFQSNLIESHSTTLYVTDVKSASERPSKDEMAS |
| SEQ ID NO: 332 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to | QLYFDYWGQGTLVTVSSASTKGPSVFPLAPCSRSTSESTAALGCLVKDYFP |
| SIGLEC10 ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSMDGRFWIRVQESVMVPEGLCISVPCSFSYPRQDWTGSTPAYGYWFK |
| | AVTETTKGAPVATNHQSREVEMSTRGRFQLTGDPAKGNCSLVIRDAQMQD |
| | ESQYFFRVERGSYVRYNFMNDGFFLKVTALTQKPDVYIPETLEPGQPVTVI |
| | CVFNWAFEECPPPSFSWTGAALSSQGTKPTTSHFSVLSFTPRPQDHNTDLTC |
| | HVDFSRKGVSAQRTVRLRVAYAPRDLVISISRDNTPALEPQPQGNVPYLEA |
| | QKGQFLRLLCAADSQPPATLSWVLQNRVLSSSHPWGPRPLGLELPGVKAG |
| | DSGRYTCRAENRLGSQQRALDLSVQYPPENLRVMVSQANRTVLENLGNGT |
| | SLPVLEGQSLCLVCVTHSSPPARLSWTQRGQVLSPSQPSDPGVLELPRVQVE |
| | HEGEFTCHARHPLGSQHVSLSLSVHYSPKLLGPSCSWEAEGLHCSCSSQASP |
| | APSLRWWLGEELLEGNSSQDSFEVTPSSAGPWANSSLSLHGGLSSGLRLRC |
| | EAWNVHGAQSGSILQLPDKKGLIST |
| SEQ ID NO: 333 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to SIRPa | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSEEELQVIQPDKSVLVAAGETATLRCTATSLIPVGPIQWFRGAGPGREL |
| | IYNQKEGHFPRVTTVSDLTKRNNMDFSIRIGNITPADAGTYYCVKFRKGSPD |
| | DVEFKSGAGTELSVRAKPS |
| SEQ ID NO: 334 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to TGFbR | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSTIPPHVQKSVNNDMIVTDNNGAVKFPQLCKFCDVRFSTCDNQKSCM |
| | SNCSITSICEKPQEVCVAVWRKNDENITLETVCHDPKLPYHDFILEDAASPK |
| | CIMKEKKKPGETFFMCSCSSDECNDNIIFSEEYNTSNPD |
| SEQ ID NO: 335 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to TIM3 | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSSEVEYRAEVGQNAYLPCFYTPAAPGNLVPVCWGKGACPVFECGNV |
| | VLRTDERDVNYWTSRYWLNGDFRKGDVSLTIENVTLADSGIYCCRIQIPGI |
| | MNDEKFNLKLVIKPAKVTPAPTRQRDFTAAFPRMLTTRGHGPAETQTLGSL |
| | PDINLTQISTLANELRDSRLANDLRDSGATIRIG |
| SEQ ID NO: 336 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to VEGFR | QLYFDYWGQGTLVTVSSASTKGPSVFPLAPCSRSTSESTAALGCLVKDYFP |
| ECD | EPVTVSWNSGALTSGVHTFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSSDTGRPFVEMYSEIPEIIHMTEGRELVIPCRVTSPNITVTLKKFPLDTLI |
| | PDGKRIIWDSRKGFIISNATYKEIGLLTCEATVNGHLYKTNYLTHRQTNTIID |
| | VVLSPSHGIELSVGEKLVLNCTARTELNVGIDFNWEYPSSKHQHKKLVNRD |
| | LKTQSGSEMKKFLSTLTIDGVTRSDQGLYTCAASSGLMTKKNSTFVRVHEK |
| SEQ ID NO: 337 | QVQLVQSGAEVKKPGSSVKVSCKASGYTFTDQTIHWMRQAPGQGLEWIG |
| anti-IL23 HC | YIYPRDDSPKYNENFKGKVTITADKSTSTAYMELSSLRSEDTAVYYCAIPDR |
| fused to BTLA | SGYAWFIYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSGG |
| | GGSGGGGSKESCDVQLYIKRQSEHSILAGDPFELECPVKYCANRPHVTWCK |
| | LNGTTCVKLEDRQTSWKEEKNISFFILHEEPVLPNDNGSYRCSANFQSNLIE |
| | SHSTTLYVTDVKSASERPSKDEMAS |
| SEQ ID NO: 338 | DIQMTQSPSSLSASVGDRVTITCKASRDVAIAVAWYQQKPGKVPKLLIYWA |
| anti-IL23 LC | STRHTGVPSRFSGSGSRTDFTLTISSLQPEDVADYFCHQYSSYPFTFGSGTKL |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 339 | DIQMTQSPSSLSASVGDRVTITCKASRDVAIAVAWYQQKPGKVPKLLIYWA |
| anti-IL23 LC | STRHTGVPSRFSGSGSRTDFTLTISSLQPEDVADYFCHQYSSYPFTFGSGTKL |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 340 | DIQMTQSPSSLSASVGDRVTITCKASRDVAIAVAWYQQKPGKVPKLLIYWA |
| anti-IL23 LC | STRHTGVPSRFSGSGSRTDFTLTISSLQPEDVADYFCHQYSSYPFTFGSGTKL |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 341 | DIQMTQSPSSLSASVGDRVTITCKASRDVAIAVAWYQQKPGKVPKLLIYWA |
| anti-IL23 LC | STRHTGVPSRFSGSGSRTDFTLTISSLQPEDVADYFCHQYSSYPFTFGSGTKL |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 342 | QVQLVQSGAEVKKPGSSVKVSCKASGYTFTDQTIHWMRQAPGQGLEWIG |
| anti-IL23 HC | YIYPRDDSPKYNENFKGKVTITADKSTSTAYMELSSLRSEDTAVYYCAIPDR |
| fused to PD1 ECD | SGYAWFIYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSGG |
| | GGSGGGGSPGWFLDSPDRPWNPPTFSPALLVVTEGDNATFTCSFSNTSESFV |
| | LNWYRMSPSNQTDKLAAFPEDRSQPGQDCRFRVTQLPNGRDFHMSVVRA |
| | RRNDSGTYLCGAISLAPKAQIKESLRAELRVTERRAEVPTAHPSPSPRPAGQ |
| | FQTLV |
| SEQ ID NO: 343 | DIQMTQSPSSLSASVGDRVTITCKASRDVAIAVAWYQQKPGKVPKLLIYWA |
| anti-IL23 LC | STRHTGVPSRFSGSGSRTDFTLTISSLQPEDVADYFCHQYSSYPFTFGSGTKL |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 344 | QVQLVQSGAEVKKPGSSVKVSCKASGYTFTDQTIHWMRQAPGQGLEWIG |
| anti-IL23 HC | YIYPRDDSPKYNENFKGKVTITADKSTSTAYMELSSLRSEDTAVYYCAIPDR |
| fused to | SGYAWFIYWGQGTLVTVSSASTKGPSVFPLAPSSKSTSGGTAALGCLVKDY |
| SIGLEC10 ECD | FPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSGG |
| | GGSGGGGSMDGRFWIRVQESVMVPEGLCISVPCSFSYPRQDWTGSTPAYG |
| | YWFKAVTETTKGAPVATNHQSREVEMSTRGRFQLTGDPAKGNCSLVIRDA |
| | QMQDESQYFFRVERGSYVRYNFMNDGFFLKVTALTQKPDVYIPETLEPGQ |
| | PVTVICVFNWAFEECPPPSFSWTGAALSSQGTKPTTSHFSVLSFTPRPQDHN |
| | TDLTCHVDFSRKGVSAQRTVRLRVAYAPRDLVISISRDNTPALEPQPQGNV |
| | PYLEAQKGQFLRLLCAADSQPPATLSWVLQNRVLSSSHPWGPRPLGLELPG |
| | VKAGDSGRYTCRAENRLGSQQRALDLSVQYPPENLRVMVSQANRTVLEN |
| | LGNGTSLPVLEGQSLCLVCVTHSSPPARLSWTQRGQVLSPSQPSDPGVLELP |
| | RVQVEHEGEFTCHARHPLGSQHVSLSLSVHYSPKLLGPSCSWEAEGLHCSC |
| | SSQASPAPSLRWWLGEELLEGNSSQDSFEVTPSSAGPWANSSLSLHGGLSS |
| | GLRLRCEAWNVHGAQSGSILQLPDKKGLIST |
| SEQ ID NO: 345 | QVQLVQSGAEVKKPGSSVKVSCKASGYTFTDQTIHWMRQAPGQGLEWIG |
| anti-IL23 HC | YIYPRDDSPKYNENFKGKVTITADKSTSTAYMELSSLRSEDTAVYYCAIPDR |
| fused to SIRPa | SGYAWFIYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSGG |
| | GGSGGGGSEEELQVIQPDKSVLVAAGETATLRCTATSLIPVGPIQWFRGAGP |
| | GRELIYNQKEGHFPRVTTVSDLTKRNNMDFSIRIGNITPADAGTYYCVKFR |
| | KGSPDDVEFKSGAGTELSVRAKPS |
| SEQ ID NO: 346 | QVQLVQSGAEVKKPGSSVKVSCKASGYTFTDQTIHWMRQAPGQGLEWIG |
| anti-IL23 HC | YIYPRDDSPKYNENFKGKVTITADKSTSTAYMELSSLRSEDTAVYYCAIPDR |
| fused to TGFbR | SGYAWFIYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSGG |
| | GGSGGGGSTIPPHVQKSVNNDMIVTDNNGAVKFPQLCKFCDVRFSTCDNQ |
| | KSCMSNCSITSICEKPQEVCVAVWRKNDENITLETVCHDPKLPYHDFILEDA |
| | ASPKCIMKEKKKPGETFFMCSCSSDECNDNIIFSEEYNTSNPD |
| SEQ ID NO: 347 | QVQLVQSGAEVKKPGSSVKVSCKASGYTFTDQTIHWMRQAPGQGLEWIG |
| anti-IL23 HC | YIYPRDDSPKYNENFKGKVTITADKSTSTAYMELSSLRSEDTAVYYCAIPDR |
| fused to TIM3 | SGYAWFIYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSGG |
| | GGSGGGGSSEVEYRAEVGQNAYLPCFYTPAAPGNLVPVCWGKGACPVFEC |
| | GNVVLRTDERDVNYWTSRYWLNGDFRKGDVSLTIENVTLADSGIYCCRIQI |
| | PGIMNDEKFNLKLVIKPAKVTPAPTRQRDFTAAFPRMLTTRGHGPAETQTL |
| | GSLPDINLTQISTLANELRDSRLANDLRDSGATIRIG |
| SEQ ID NO: 348 | QVQLVQSGAEVKKPGSSVKVSCKASGYTFTDQTIHWMRQAPGQGLEWIG |
| anti-IL23 HC | YIYPRDDSPKYNENFKGKVTITADKSTSTAYMELSSLRSEDTAVYYCAIPDR |
| fused to VEGFR | SGYAWFIYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPEAAGGPSVFLFPPKPKDTLM |
| | ISRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSGG |
| | GGSGGGGSSDTGRPFVEMYSEIPEIIHMTEGRELVIPCRVTSPNITVTLKKFP |
| | LDTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |
| SEQ ID NO: 349 | QVQLVESGGGVVQPGRSLRLSCAASGFTFSVYGMNWVRQAPGKGLEWVA |
| anti-IL1b HC | IIWYDGDNQYYADSVKGRFTISRDNSKNTLYLQMNGLRAEDTAVYYCARD |
| fused to BTLA | LRTGPFDYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSKESCDVQLYIKRQSEHSILAGDPFELECPVKYCANRPHVTWC |
| | KLNGTTCVKLEDRQTSWKEEKNISFFILHEEPVLPNDNGSYRCSANFQSNLI |
| | ESHSTTLYVTDVKSASERPSKDEMAS |
| SEQ ID NO: 350 | EIVLTQSPDFQSVTPKEKVTITCRASQSIGSSLHWYQQKPDQSPKLLIKYASQ |
| anti-IL1b LC | SFSGVPSRFSGSGSGTDFTLTINSLEAEDAAAYYCHQSSSLPFTFGPGTKVDI |
| fused to PD1 ECD | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKS |
| | FNRGECGGGGSGGGGSGGGGSPGWFLDSPDRPWNPPTFSPALLVVTEGDN |
| | ATFTCSFSNTSESFVLNWYRMSPSNQTDKLAAFPEDRSQPGQDCRFRVTQL |
| | PNGRDFHMSVVRARRNDSGTYLCGAISLAPKAQIKESLRAELRVTERRAEV |
| | PTAHPSPSPRPAGQFQTLV |
| SEQ ID NO: 351 | EIVLTQSPDFQSVTPKEKVTITCRASQSIGSSLHWYQQKPDQSPKLLIKYASQ |
| anti-IL1b LC | SFSGVPSRFSGSGSGTDFTLTINSLEAEDAAAYYCHQSSSLPFTFGPGTKVDI |
| fused to | KRTVAAPSVFIFPPSDEQLKSGTASVVCLLNNFYPREAKVQWKVDNALQSG |
| SIGLEC10 ECD | NSQESVTEQDSKDSTYSLSSTLTLSKADYEKHKVYACEVTHQGLSSPVTKS |
| | FNRGECGGGGSGGGGSGGGGSMDGRFWIRVQESVMVPEGLCISVPCSFSY |
| | PRQDWTGSTPAYGYWFKAVTETTKGAPVATNHQSREVEMSTRGRFQLTG |
| | DPAKGNCSLVIRDAQMQDESQYFFRVERGSYVRYNFMNDGFFLKVTALTQ |
| | KPDVYIPETLEPGQPVTVICVFNWAFEECPPPSFSWTGAALSSQGTKPTTSH |
| | FSVLSFTPRPQDHNTDLTCHVDFSRKGVSAQRTVRLRVAYAPRDLVISISRD |
| | NTPALEPQPQGNVPYLEAQKGQFLRLLCAADSQPPATLSWVLQNRVLSSSH |
| | PWGPRPLGLELPGVKAGDSGRYTCRAENRLGSQQRALDLSVQYPPENLRV |
| | MVSQANRTVLENLGNGTSLPVLEGQSLCLVCVTHSSPPARLSWTQRGQVL |
| | SPSQPSDPGVLELPRVQVEHEGEFTCHARHPLGSQHVSLSLSVHYSPKLLGP |
| | SCSWEAEGLHCSCSSQASPAPSLRWWLGEELLEGNSSQDSFEVTPSSAGPW |
| | ANSSLSLHGGLSSGLRLRCEAWNVHGAQSGSILQLPDKKGLIST |
| SEQ ID NO: 352 | EIVLTQSPDFQSVTPKEKVTITCRASQSIGSSLHWYQQKPDQSPKLLIKYASQ |
| anti-IL1b LC | SFSGVPSRFSGSGSGTDFTLTINSLEAEDAAAYYCHQSSSLPFTFGPGTKVDI |
| fused to SIRPa | KRTVAAPSVFIFPPSDEQLKSGTASVVCLLNNFYPREAKVQWKVDNALQSG |
| ECD | NSQESVTEQDSKDSTYSLSSTLTLSKADYEKHKVYACEVTHQGLSSPVTKS |
| | FNRGECGGGGSGGGGSGGGGSEEELQVIQPDKSVLVAAGETATLRCTATSL |
| | IPVGPIQWFRGAGPGRELIYNQKEGHFPRVTTVSDLTKRNNMDFSIRIGNITP |
| | ADAGTYYCVKFRKGSPDDVEFKSGAGTELSVRAKPS |
| SEQ ID NO: 353 | EIVLTQSPDFQSVTPKEKVTITCRASQSIGSSLHWYQQKPDQSPKLLIKYASQ |
| anti-IL1b LC | SFSGVPSRFSGSGSGTDFTLTINSLEAEDAAAYYCHQSSSLPFTFGPGTKVDI |
| fused to TIM3 | KRTVAAPSVFIFPPSDEQLKSGTASVVCLLNNFYPREAKVQWKVDNALQSG |
| ECD | NSQESVTEQDSKDSTYSLSSTLTLSKADYEKHKVYACEVTHQGLSSPVTKS |
| | FNRGECGGGGSGGGGSGGGGSSEVEYRAEVGQNAYLPCFYTPAAPGNLVP |
| | VCWGKGACPVFECGNVVLRTDERDVNYWTSRYWLNGDFRKGDVSLTIEN |
| | VTLADSGIYCCRIQIPGIVINDEKFNLKLVIKPAKVTPAPTRQRDFTAAFPRM |
| | LTTRGHGPAETQTLGSLPDINLTQISTLANELRDSRLANDLRDSGATIRIG |
| SEQ ID NO: 354 | QVQLVESGGGVVQPGRSLRLSCAASGFTFSVYGMNWVRQAPGKGLEWVA |
| anti-IL1b HC | IIWYDGDNQYYADSVKGRFTISRDNSKNTLYLQMNGLRAEDTAVYYCARD |
| fused to PD1 ECD | LRTGPFDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSPGWFLDSPDRPWNPPTFSPALLVVTEGDNATFTCSFSNTSESF |
| | VLNWYRMSPSNQTDKLAAFPEDRSQPGQDCRFRVTQLPNGRDFHMSVVR |
| | ARRNDSGTYLCGAISLAPKAQIKESLRAELRVTERRAEVPTAHPSPSPRPAG |
| | QFQTLV |
| SEQ ID NO: 355 | EIVLTQSPDFQSVTPKEKVTITCRASQSIGSSLHWYQQKPDQSPKLLIKYASQ |
| anti-IL1b LC | SFSGVPSRFSGSGSGTDFTLTINSLEAEDAAAYYCHQSSSLPFTFGPGTKVDI |
| fused to BTLA | KRTVAAPSVFIFPPSDEQLKSGTASVVCLLNNFYPREAKVQWKVDNALQSG |
| ECD | NSQESVTEQDSKDSTYSLSSTLTLSKADYEKHKVYACEVTHQGLSSPVTKS |
| | FNRGECGGGGSGGGGSGGGGSKESCDVQLYIKRQSEHSILAGDPFELECPV |
| | KYCANRPHVTWCKLNGTTCVKLEDRQTSWKEEKNISFFILHFEPVLPNDNG |
| | SYRCSANFQSNLIESHSTTLYVTDVKSASERPSKDEMAS |
| SEQ ID NO: 356 | QVQLVESGGGVVQPGRSLRLSCAASGFTFSVYGMNWVRQAPGKGLEWVA |
| anti-IL1b HC | IIWYDGDNQYYADSVKGRFTISRDNSKNTLYLQMNGLRAEDTAVYYCARD |
| fused to | LRTGPFDYWGQGTLVTVSSASTKGPSVFPLAPSSKSTSGGTAALGCLVKDY |
| SIGLEC10 ECD | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSMDGRFWIRVQESVMVPEGLCISVPCSFSYPRQDWTGSTPAY |
| | GYWFKAVTETTKGAPVATNHQSREVEMSTRGRFQLTGDPAKGNCSLVIRD |
| | AQMQDESQYFFRVERGSYVRYNFMNDGFFLKVTALTQKPDVYIPETLEPG |
| | QPVTVICVFNWAFEECPPPSFSWTGAALSSQGTKPTTSHFSVLSFTPRPQDH |
| | NTDLTCHVDFSRKGVSAQRTVRLRVAYAPRDLVISISRDNTPALEPQPQGN |
| | VPYLEAQKGQFLRLLCAADSQPPATLSWVLQNRVLSSSHPWGPRPLGLELP |
| | GVKAGDSGRYTCRAENRLGSQQRALDLSVQYPPENLRVMVSQANRTVLE |
| | NLGNGTSLPVLEGQSLCLVCVTHSSPPARLSWTQRGQVLSPSQPSDPGVLE |
| | LPRVQVEHEGEFTCHARHPLGSQHVSLSLSVHYSPKLLGPSCSWEAEGLHC |
| | SCSSQASPAPSLRWWLGEELLEGNSSQDSFEVTPSSAGPWANSSLSLHGGLS |
| | SGLRLRCEAWNVHGAQSGSILQLPDKKGLIST |
| SEQ ID NO: 357 | QVQLVESGGGVVQPGRSLRLSCAASGFTFSVYGMNWVRQAPGKGLEWVA |
| anti-IL1b HC | IIWYDGDNQYYADSVKGRFTISRDNSKNTLYLQMNGLRAEDTAVYYCARD |
| fused to SIRPa | LRTGPFDYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSEEELQVIQPDKSVLVAAGETATLRCTATSLIPVGPIQWFRGA |
| | GPGRELIYNQKEGHFPRVTTVSDLTKRNNMDFSIRIGNITPADAGTYYCVKF |
| | RKGSPDDVEFKSGAGTELSVRAKPS |
| SEQ ID NO: 358 | QVQLVESGGGVVQPGRSLRLSCAASGFTFSVYGMNWVRQAPGKGLEWVA |
| anti-IL1b HC | IIWYDGDNQYYADSVKGRFTISRDNSKNTLYLQMNGLRAEDTAVYYCARD |
| fused to TGFbR | LRTGPFDYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSTIPPHVQKSVNNDMIVTDNNGAVKFPQLCKFCDVRFSTCDN |
| | QKSCMSNCSITSICEKPQEVCVAVWRKNDENITLETVCHDPKLPYHDFILED |
| | AASPKCIMKEKKKPGETFFMCSCSSDECNDNIIFSEEYNTSNPD |
| SEQ ID NO: 359 | QVQLVESGGGVVQPGRSLRLSCAASGFTFSVYGMNWVRQAPGKGLEWVA |
| anti-IL1b HC | IIWYDGDNQYYADSVKGRFTISRDNSKNTLYLQMNGLRAEDTAVYYCARD |
| fused to TIM3 | LRTGPFDYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSSEVEYRAEVGQNAYLPCFYTPAAPGNLVPVCWGKGACPVF |
| | ECGNVVLRTDERDVNYWTSRYWLNGDFRKGDVSLTIENVTLADSGIYCCR |
| | IQIPGIMNDEKFNLKLVIKPAKVTPAPTRQRDFTAAFPRMLFERGHGPAETQ |
| | TLGSLPDINLTQISTLANELRDSRLANDLRDSGATIRIG |
| SEQ ID NO: 360 | QVQLVESGGGVVQPGRSLRLSCAASGFTFSVYGMNWVRQAPGKGLEWVA |
| anti-IL1b HC | IIWYDGDNQYYADSVKGRFTISRDNSKNTLYLQMNGLRAEDTAVYYCARD |
| fused to VEGFR | LRTGPFDYWGQGTLVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKRVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSSDTGRPFVEMYSEIPEIIHMTEGRELVIPCRVTSPNITVTLKKF |
| | PLDTLIPDGKRIIWDSRKGFIISNATYKEIGLLTCEATVNGHLYKTNYLTHRQ |
| | TNTIIDVVLSPSHGIELSVGEKLVLNCTARTELNVGIDFNWEYPSSKHQHKK |
| | LVNRDLKTQSGSEMKKFLSTLTIDGVTRSDQGLYTCAASSGLMTKKNSTFV |
| | RVHEK |
| SEQ ID NO: 361 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to BTLA | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSKESCDVQLYIKRQSEHSILAGDPFELECPVKYCANR |
| | PHVTWCKLNGTTCVKLEDRQTSWKEEKNISFFILHEEPVLPNDNGSYRCSA |
| | NFQSNLIESHSTTLYVTDVKSASERPSKDEMAS |
| SEQ ID NO: 362 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 363 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 364 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 365 | DIQMTQSPSSLSASVGDRVTITCSASQDISNYLNWYQQKPGKAPKVLIYFTS |
| anti- VEGF LC | SLHSGVPSRFSGSGSGTDFTLTISSLQPEDFATYYCQQYSTVPWTFGQGTKV |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 366 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to PD1 ECD | PHYYGSSHWYFDVWGQGTLVTVSSASTKGPSVFPLAPSSKSTSGGTAALG |
| | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSPGWFLDSPDRPWNPPTFSPALLVVTEGDNATFTCSF |
| | SNTSESFVLNWYRMSPSNQTDKLAAFPEDRSQPGQDCRFRVTQLPNGRDF |
| | HMSVVRARRNDSGTYLCGAISLAPKAQIKESLRAELRVTERRAEVPTAHPS |
| | PSPRPAGQFQTLV |
| SEQ ID NO: 367 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 368 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to | PHYYGSSHWYFDVWGQGTLVTVSSASTKGPSVFPLAPSSKSTSGGTAALG |
| SIGLEC10 ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSMDGRFWIRVQESVMVPEGLCISVPCSFSYPRQDWT |
| | GSTPAYGYWFKAVTETTKGAPVATNHQSREVEMSTRGRFQLTGDPAKGN |
| | CSLVIRDAQMQDESQYFFRVERGSYVRYNFMNDGFFLKVTALTQKPDVYI |
| | PETLEPGQPVTVICVFNWAFEECPPPSFSWTGAALSSQGTKPTTSHFSVLSFT |
| | PRPQDHNTDLTCHVDFSRKGVSAQRTVRLRVAYAPRDLVISISRDNTPALE |
| | PQPQGNVPYLEAQKGQFLRLLCAADSQPPATLSWVLQNRVLSSSHPWGPR |
| | PLGLELPGVKAGDSGRYTCRAENRLGSQQRALDLSVQYPPENLRVMVSQA |
| | NRTVLENLGNGTSLPVLEGQSLCLVCVTHSSPPARLSWTQRGQVLSPSQPS |
| | DPGVLELPRVQVEHEGEFTCHARHPLGSQHVSLSLSVHYSPKLLGPSCSWE |
| | AEGLHCSCSSQASPAPSLRWWLGEELLEGNSSQDSFEVTPSSAGPWANSSL |
| | SLHGGLSSGLRLRCEAWNVHGAQSGSILQLPDKKGLIST |
| SEQ ID NO: 369 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to SIRPa | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSEEELQVIQPDKSVLVAAGETATLRCTATSLIPVGPIQ |
| | WFRGAGPGRELIYNQKEGHFPRVTTVSDLTKRNNMDFSIRIGNITPADAGT |
| | YYCVKFRKGSPDDVEFKSGAGTELSVRAKPS |
| SEQ ID NO: 370 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to TGFbR | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSTIPPHVQKSVNNDMIVTDNNGAVKFPQLCKFCDVR |
| | FSTCDNQKSCMSNCSITSICEKPQEVCVAVWRKNDENITLETVCHDPKLPY |
| | HDFILEDAASPKCIMKEKKKPGETFFMCSCSSDECNDNIIFSEEYNTSNPD |
| SEQ ID NO: 371 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to TIM3 | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSSEVEYRAEVGQNAYLPCFYTPAAPGNLVPVCWGK |
| | GACPVFECGNVVLRTDERDVNYWTSRYWLNGDFRKGDVSLTIENVTLADS |
| | GIYCCRIQIPGIMNDEKFNLKLVIKPAKVTPAPTRQRDFTAAFPRMLTTRGH |
| | GPAETQTLGSLPDINLTQISTLANELRDSRLANDLRDSGATIRIG |
| SEQ ID NO: 372 | EVQLVQSGGGLVKPGGSLRLSCAASGFTFSSYSMNWVRQAPGKGLEWVSS |
| anti-VEGFR HC | ISSSSSYIYYADSVKGRFTISRDNAKNSLYLQMNSLRAEDTAVYYCARVTD |
| fused to BTLA | AFDIWGQGTMVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSKESCDVQLYIKRQSEHSILAGDPFELECPVKYCANRPHVTWCKLN |
| | GTTCVKLEDRQTSWKEEKNISFFILHFEPVLPNDNGSYRCSANFQSNLIESHS |
| | TTLYVTDVKSASERPSKDEMAS |
| SEQ ID NO: 373 | DIQMTQSPSSVSASIGDRVTITCRASQGIDNWLGWYQQKPGKAPKLLIYDA |
| anti-VEGFR LC | SNLDTGVPSRFSGSGSGTYFTLTISSLQAEDFAVYFCQQAKAFPPTFGGGTK |
| fused to PD1 ECD | VDIKGTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPV |
| | TKSFNRGECGGGGSGGGGSGGGGSPGWFLDSPDRPWNPPTFSPALLVVTE |
| | GDNATFTCSFSNTSESFVLNWYRMSPSNQTDKLAAFPEDRSQPGQDCRFRV |
| | TQLPNGRDFHMSVVRARRNDSGTYLCGAISLAPKAQIKESLRAELRVTERR |
| | AEVPTAHPSPSPRPAGQFQTLV |
| SEQ ID NO: 374 | DIQMTQSPSSVSASIGDRVTITCRASQGIDNWLGWYQQKPGKAPKLLIYDA |
| anti-VEGFR LC | SNLDTGVPSRFSGSGSGTYFTLTISSLQAEDFAVYFCQQAKAFPPTFGGGTK |
| fused to | VDIKGTVAAPSVFIFPPSDEQLKSGTASVVCLLNNFYPREAKVQWKVDNAL |
| SIGLEC10 ECD | QSGNSQESVTEQDSKDSTYSLSSTLTLSKADYEKHKVYACEVTHQGLSSPV |
| | TKSFNRGECGGGGSGGGGSGGGGSMDGRFWIRVQESVMVPEGLCISVPCS |
| | FSYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 375 | DIQMTQSPSSVSASIGDRVTITCRASQGIDNWLGWYQQKPGKAPKLLIYDA |
| anti-VEGFR LC | SNLDTGVPSRFSGSGSGTYFTLTISSLQAEDFAVYFCQQAKAFPPTFGGGTK |
| fused to SIRPa | VDIKGTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSEEELQVIQPDKSVLVAAGETATLRCT |
| | ATSLIPVGPIQWFRGAGPGRELIYNQKEGHFPRVTTVSDLTKRNNMDFSIRI |
| | GNITPADAGTYYCVKFRKGSPDDVEFKSGAGTELSVRAKPS |
| SEQ ID NO: 376 | DIQMTQSPSSVSASIGDRVTITCRASQGIDNWLGWYQQKPGKAPKLLIYDA |
| anti-VEGFR LC | SNLDTGVPSRFSGSGSGTYFTLTISSLQAEDFAVYFCQQAKAFPPTFGGGTK |
| fused to TIM3 | VDIKGTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSSEVEYRAEVGQNAYLPCFYTPAAPGN |
| | LVPVCWGKGACPVFECGNVVLRTDERDVNYWTSRYWLNGDFRKGDVSL |
| | TIENVTLADSGIYCCRIQIPGIMNDEKFNLKLVIKPAKVTPAPTRQRDFTAAF |
| | PRMLTTRGHGPAETQTLGSLPDINLTQISTLANELRDSRLANDLRDSGATIRI |
| | G |
| SEQ ID NO: 377 | EVQLVQSGGGLVKPGGSLRLSCAASGFTFSSYSMNWVRQAPGKGLEWVSS |
| anti-VEGFR HC | ISSSSSYIYYADSVKGRFTISRDNAKNSLYLQMNSLRAEDTAVYYCARVTD |
| fused to PD1 ECD | AFDIWGQGTMVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSPGWFLDSPDRPWNPPTFSPALLVVTEGDNATFTCSFSNTSESFVLN |
| | WYRMSPSNQTDKLAAFPEDRSQPGQDCRFRVTQLPNGRDFHMSVVRARR |
| | NDSGTYLCGAISLAPKAQIKESLRAELRVTERRAEVPTAHPSPSPRPAGQFQ |
| | TLV |
| SEQ ID NO: 378 | DIQMTQSPSSVSASIGDRVTITCRASQGIDNWLGWYQQKPGKAPKLLIYDA |
| anti-VEGFR LC | SNLDTGVPSRFSGSGSGTYFTLTISSLQAEDFAVYFCQQAKAFPPTFGGGTK |
| fused to BTLA | VDIKGTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSKESCDVQLYIKRQSEHSILAGDPFELE |
| | CPVKYCANRPHVTWCKLNGTTCVKLEDRQTSWKEEKNISFFILHFEPVLPN |
| | DNGSYRCSANFQSNLIESHSTTLYVTDVKSASERPSKDEMAS |
| SEQ ID NO: 379 | EVQLVQSGGGLVKPGGSLRLSCAASGFTFSSYSMNWVRQAPGKGLEWVSS |
| anti-VEGFR HC | ISSSSSYIYYADSVKGRFTISRDNAKNSLYLQMNSLRAEDTAVYYCARVTD |
| fused to | AFDIWGQGTMVTVSSASTKGPSVFPLAPSSKSTSGGTAALGCLVKDYFPEP |
| SIGLEC10 ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSMDGRFWIRVQESVMVPEGLCISVPCSFSYPRQDWTGSTPAYGYW |
| | FKAVTETTKGAPVATNHQSREVEMSTRGRFQLTGDPAKGNCSLVIRDAQM |
| | QDESQYFFRVERGSYVRYNFMNDGFFLKVTALTQKPDVYIPETLEPGQPVT |
| | VICVFNWAFEECPPPSFSWTGAALSSQGTKPTTSHFSVLSFTPRPQDHNTDL |
| | TCHVDFSRKGVSAQRTVRLRVAYAPRDLVISISRDNTPALEPQPQGNVPYL |
| | EAQKGQFLRLLCAADSQPPATLSWVLQNRVLSSSHPWGPRPLGLELPGVK |
| | AGDSGRYTCRAENRLGSQQRALDLSVQYPPENLRVMVSQANRTVLENLG |
| | NGTSLPVLEGQSLCLVCVTHSSPPARLSWTQRGQVLSPSQPSDPGVLELPRV |
| | QVEHEGEFTCHARHPLGSQHVSLSLSVHYSPKLLGPSCSWEAEGLHCSCSS |
| | QASPAPSLRWWLGEELLEGNSSQDSFEVTPSSAGPWANSSLSLHGGLSSGL |
| | RLRCEAWNVHGAQSGSILQLPDKKGLIST |
| SEQ ID NO: 380 | EVQLVQSGGGLVKPGGSLRLSCAASGFTFSSYSMNWVRQAPGKGLEWVSS |
| anti-VEGFR HC | ISSSSSYIYYADSVKGRFTISRDNAKNSLYLQMNSLRAEDTAVYYCARVTD |
| fused to SIRPa | AFDIWGQGTMVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSEEELQVIQPDKSVLVAAGETATLRCTATSLIPVGPIQWFRGAGPGR |
| | ELIYNQKEGHFPRVTTVSDLTKRNNMDFSIRIGNITPADAGTYYCVKFRKGS |
| | PDDVEFKSGAGTELSVRAKPS |
| SEQ ID NO: 381 | EVQLVQSGGGLVKPGGSLRLSCAASGFTFSSYSMNWVRQAPGKGLEWVSS |
| anti-VEGFR HC | ISSSSSYIYYADSVKGRFTISRDNAKNSLYLQMNSLRAEDTAVYYCARVTD |
| fused to TGFbR | AFDIWGQGTMVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSTIPPHVQKSVNNDMIVTDNNGAVKFPQLCKFCDVRFSTCDNQKS |
| | CMSNCSITSICEKPQEVCVAVWRKNDENITLETVCHDPKLPYHDFILEDAAS |
| | PKCIMKEKKKPGETFFMCSCSSDECNDNIIFSEEYNTSNPD |
| SEQ ID NO: 382 | EVQLVQSGGGLVKPGGSLRLSCAASGFTFSSYSMNWVRQAPGKGLEWVSS |
| anti-VEGFR HC | ISSSSSYIYYADSVKGRFTISRDNAKNSLYLQMNSLRAEDTAVYYCARVTD |
| fused to TIM3 | AFDIWGQGTMVTVSSASTKGPSVFPLAPSSKSTSGGTAALGCLVKDYFPEP |
| ECD | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSEVEYRAEVGQNAYLPCFYTPAAPGNLVPVCWGKGACPVFECGN |
| | VVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGS |
| | LPDINLTQISTLANELRDSRLANDLRDSGATIRIG |
| SEQ ID NO: 383 | QVQLQQPGAELVKPGASVMMSCKASGYTFTNYNMHWVKQTPGQGLEWI |
| anti-CD47 HC | GTIYPGNDDTSYNQKFKDKATLTADKSSSAAYMQLSSLTSEDSAVYYCAR |
| fused to BTLA | GGYRAMDYWGQTSVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSKESCDVQLYIKRQSEHSILAGDPFELECPVKYCANRPHVTWC |
| | KLNGTTCVKLEDRQTSWKEEKNISFFILHEEPVLPNDNGSYRCSANFQSNLI |
| | ESHSTTLYVTDVKSASERPSKDEMAS |
| SEQ ID NO: 384 | DVLMTQTPLSLPVSLGDQASISCRSSQSIVYSNGNTYLGWYLQKPGQSPKL |
| anti-CD47 LC | LIYKVSNRFSGVPDRFSGSGSGTDFTLKISRVEAEDLGVYHCFQGSHVPYTF |
| fused to PD1 ECD | GGGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQ |
| | GLSSPVTKSFNRGECGGGGSGGGGSGGGGSPGWFLDSPDRPWNPPTFSPAL |
| | LVVTEGDNATFTCSFSNTSESFVLNWYRMSPSNQTDKLAAFPEDRSQPGQD |
| | CRFRVTQLPNGRDFHMSVVRARRNDSGTYLCGAISLAPKAQIKESLRAELR |
| | VTERRAEVPTAHPSPSPRPAGQFQTLV |
| SEQ ID NO: 385 | DVLMTQTPLSLPVSLGDQASISCRSSQSIVYSNGNTYLGWYLQKPGQSPKL |
| anti-CD47 LC | LIYKVSNRFSGVPDRFSGSGSGTDFTLKISRVEAEDLGVYHCFQGSHVPYTF |
| fused to | GGGTKVEIKRTVAAPSVFIFPPSDEQLKSGTASVVCLLNNFYPREAKVQWK |
| SIGLEC10 ECD | VDNALQSGNSQESVTEQDSKDSTYSLSSTLTLSKADYEKHKVYACEVTHQ |
| | GLSSPVTKSFNRGECGGGGSGGGGSGGGGSMDGRFWIRVQESVMVPEGLC |
| | ISVPCSFSYPRQDWTGSTPAYGYWFKAVTETTKGAPVATNHQSREVEMST |
| | RGRFQLTGDPAKGNCSLVIRDAQMQDESQYFFRVERGSYVRYNFMNDGFF |
| | LKVTALTQKPDVYIPETLEPGQPVTVICVFNWAFEECPPPSFSWTGAALSSQ |
| | GTKPTTSHFSVLSFTPRPQDHNTDLTCHVDFSRKGVSAQRTVRLRVAYAPR |
| | DLVISISRDNTPALEPQPQGNVPYLEAQKGQFLRLLCAADSQPPATLSWVL |
| | QNRVLSSSHPWGPRPLGLELPGVKAGDSGRYTCRAENRLGSQQRALDLSV |
| | QYPPENLRVMVSQANRTVLENLGNGTSLPVLEGQSLCLVCVTHSSPPARLS |
| | WTQRGQVLSPSQPSDPGVLELPRVQVEHEGEFTCHARHPLGSQHVSLSLSV |
| | HYSPKLLGPSCSWEAEGLHCSCSSQASPAPSLRWWLGEELLEGNSSQDSFE |
| | VTPSSAGPWANSSLSLHGGLSSGLRLRCEAWNVHGAQSGSILQLPDKKGLI |
| | ST |
| SEQ ID NO: 386 | DVLMTQTPLSLPVSLGDQASISCRSSQSIVYSNGNTYLGWYLQKPGQSPKL |
| anti-CD47 LC | LIYKVSNRFSGVPDRFSGSGSGTDFTLKISRVEAEDLGVYHCFQGSHVPYTF |
| fused to TIM3 | GGGTKVEIKRTVAAPSVFIFPPSDEQLKSGTASVVCLLNNFYPREAKVQWK |
| ECD | VDNALQSGNSQESVTEQDSKDSTYSLSSTLTLSKADYEKHKVYACEVTHQ |
| | GLSSPVTKSFNRGECGGGGSGGGGSGGGGSSEVEYRAEVGQNAYLPCFYT |
| | PAAPGNLVPVCWGKGACPVFECGNVVLRTDERDVNYWTSRYWLNGDFR |
| | KGDVSLTIENVTLADSGIYCCRIQIPGIMNDEKFNLKLVIKPAKVTPAPTRQR |
| | DFTAAFPRMLTTRGHGPAETQTLGSLPDINLTQISTLANELRDSRLANDLRD |
| | SGATIRIG |
| SEQ ID NO: 387 | QVQLQQPGAELVKPGASVMNISCKASGYTFTNYNMHWVKQTPGQGLEWI |
| anti-CD47 HC | GTIYPGNDDTSYNQKFKDKATLTADKSSSAAYMQLSSLTSEDSAVYYCAR |
| fused to PD1 ECD | GGYRAMDYWGQTSVTVSSASTKGPSVFPLAPSSKSTSGGTAALGCLVKDY |
| | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSPGWFLDSPDRPWNPPTFSPALLVVTEGDNATFTCSFSNTSESF |
| | VLNWYRMSPSNQTDKLAAFPEDRSQPGQDCRFRVTQLPNGRDFHMSVVR |
| | ARRNDSGTYLCGAISLAPKAQIKESLRAELRVTERRAEVPTAHPSPSPRPAG |
| | QFQTLV |
| SEQ ID NO: 388 | DVLMTQTPLSLPVSLGDQASISCRSSQSIVYSNGNTYLGWYLQKPGQSPKL |
| anti-CD47 LC | LIYKVSNRFSGVPDRFSGSGSGTDFTLKISRVEAEDLGVYHCFQGSHVPYTF |
| fused to BTLA | GGGTKVEIKRTVAAPSVFIFPPSDEQLKSGTASVVCLLNNFYPREAKVQWK |
| ECD | VDNALQSGNSQESVTEQDSKDSTYSLSSTLTLSKADYEKHKVYACEVTHQ |
| | GLSSPVTKSFNRGECGGGGSGGGGSGGGGSKESCDVQLYIKRQSEHSILAG |
| | DPFELECPVKYCANRPHVTWCKLNGTTCVKLEDRQTSWKEEKNISFFILHF |
| | EPVLPNDNGSYRCSANFQSNLIESHSTTLYVTDVKSASERPSKDEMAS |
| SEQ ID NO: 389 | QVQLQQPGAELVKPGASVMMSCKASGYTFTNYNMHWVKQTPGQGLEWI |
| anti-CD47 HC | GTIYPGNDDTSYNQKFKDKATLTADKSSSAAYMQLSSLTSEDSAVYYCAR |
| fused to | GGYRAMDYWGQTSVTVSSASTKGPSVFPLAPSSKSTSGGTAALGCLVKDY |
| SIGLEC10 ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSMDGRFWIRVQESVMVPEGLCISVPCSFSYPRQDWTGSTPAY |
| | GYWFKAVTETTKGAPVATNHQSREVEMSTRGRFQLTGDPAKGNCSLVIRD |
| | AQMQDESQYFFRVERGSYVRYNFMNDGFFLKVTALTQKPDVYIPETLEPG |
| | QPVTVICVFNWAFEECPPPSFSWTGAALSSQGTKPTTSHFSVLSFTPRPQDH |
| | NTDLTCHVDFSRKGVSAQRTVRLRVAYAPRDLVISISRDNTPALEPQPQGN |
| | VPYLEAQKGQFLRLLCAADSQPPATLSWVLQNRVLSSSHPWGPRPLGLELP |
| | GVKAGDSGRYTCRAENRLGSQQRALDLSVQYPPENLRVMVSQANRTVLE |
| | NLGNGTSLPVLEGQSLCLVCVTHSSPPARLSWTQRGQVLSPSQPSDPGVLE |
| | LPRVQVEHEGEFTCHARHPLGSQHVSLSLSVHYSPKLLGPSCSWEAEGLHC |
| | SCSSQASPAPSLRWWLGEELLEGNSSQDSFEVTPSSAGPWANSSLSLHGGLS |
| | SGLRLRCEAWNVHGAQSGSILQLPDKKGLIST |
| SEQ ID NO: 390 | QVQLQQPGAELVKPGASVMMSCKASGYTFTNYNMHWVKQTPGQGLEWI |
| anti-CD47 HC | GTIYPGNDDTSYNQKFKDKATLTADKSSSAAYMQLSSLTSEDSAVYYCAR |
| fused to TGFbR | GGYRAMDYWGQTSVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSTIPPHVQKSVNNDMIVTDNNGAVKFPQLCKFCDVRFSTCDN |
| | QKSCMSNCSITSICEKPQEVCVAVWRKNDENITLETVCHDPKLPYHDFILED |
| | AASPKCIMKEKKKPGETFFMCSCSSDECNDNIIFSEEYNTSNPD |
| SEQ ID NO: 391 | QVQLQQPGAELVKPGASVMNISCKASGYTFTNYNMHWVKQTPGQGLEWI |
| anti-CD47 HC | GTIYPGNDDTSYNQKFKDKATLTADKSSSAAYMQLSSLTSEDSAVYYCAR |
| fused to TIM3 | GGYRAMDYWGQTSVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSSEVEYRAEVGQNAYLPCFYTPAAPGNLVPVCWGKGACPVF |
| | ECGNVVLRTDERDVNYWTSRYWLNGDFRKGDVSLTIENVTLADSGIYCCR |
| | IQIPGIMNDEKFNLKLVIKPAKVTPAPTRQRDFTAAFPRMLFERGHGPAETQ |
| | TLGSLPDINLTQISTLANELRDSRLANDLRDSGATIRIG |
| SEQ ID NO: 392 | QVQLQQPGAELVKPGASVMMSCKASGYTFTNYNMHWVKQTPGQGLEWI |
| anti-CD47 HC | GTIYPGNDDTSYNQKFKDKATLTADKSSSAAYMQLSSLTSEDSAVYYCAR |
| fused to VEGFR | GGYRAMDYWGQTSVTVSSASTKGPSVFPLAPSSKSTSGGTAALGCLVKDY |
| ECD | FPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGKGGGGSG |
| | GGGSGGGGSSDTGRPFVEMYSEIPEIIHMTEGRELVIPCRVTSPNITVTLKKF |
| | PLDTLIPDGKRIIWDSRKGFIISNATYKEIGLLTCEATVNGHLYKTNYLTHRQ |
| | TNTIIDVVLSPSHGIELSVGEKLVLNCTARTELNVGIDFNWEYPSSKHQHKK |
| | LVNRDLKTQSGSEMKKFLSTLTIDGVTRSDQGLYTCAASSGLMTKKNSTFV |
| | RVHEK |
| SEQ ID NO: 393 | QVQLVQSGAEVKKPGSSVKVSCKASGYTFSSNVISWVRQAPGQGLEWMG |
| anti-TGFb HC | GVIPIVDIANYAQRFKGRVTITADESTSTTYMELSSLRSEDTAVYYCASTLG |
| fused to BTLA | LVLDAMDYWGQGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPSCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSKESCDVQLYIKRQSEHSILAGDPFELECPVKYCANRPHVTWCKL |
| | NGTTCVKLEDRQTSWKEEKNISFFILHEEPVLPNDNGSYRCSANFQSNLIES |
| | HSTTLYVTDVKSASERPSKDEMAS |
| SEQ ID NO: 394 | ETVLTQSPGTLSLSPGERATLSCRASQSLGSSYLAWYQQKPGQAPRLLIYGA |
| anti-TGFb LC | SSRAPGIPDRFSGSGSGTDFTLTISRLEPEDFAVYYCQQYADSPITFGQGTRL |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 395 | ETVLTQSPGTLSLSPGERATLSCRASQSLGSSYLAWYQQKPGQAPRLLIYGA |
| anti-TGFb LC | SSRAPGIPDRFSGSGSGTDFTLTISRLEPEDFAVYYCQQYADSPITFGQGTRL |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 396 | ETVLTQSPGTLSLSPGERATLSCRASQSLGSSYLAWYQQKPGQAPRLLIYGA |
| anti-TGFb LC | SSRAPGIPDRFSGSGSGTDFTLTISRLEPEDFAVYYCQQYADSPITFGQGTRL |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 397 | ETVLTQSPGTLSLSPGERATLSCRASQSLGSSYLAWYQQKPGQAPRLLIYGA |
| anti-TGFb LC | SSRAPGIPDRFSGSGSGTDFTLTISRLEPEDFAVYYCQQYADSPITFGQGTRL |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 398 | QVQLVQSGAEVKKPGSSVKVSCKASGYTFSSNVISWVRQAPGQGLEWMG |
| anti-TGFb HC | GVIPIVDIANYAQRFKGRVTITADESTSTTYMELSSLRSEDTAVYYCASTLG |
| fused to PD1 ECD | LVLDAMDYWGQGTLVTVSSASTKGPSVFPLAPCSRSTSESTAALGCLVKD |
| | YFPEPVTVSWNSGALTSGVHTFPAVLQSSGLYSLSSVVTVPSSSLGTKTYTC |
| | NVDHKPSNTKVDKRVESKYGPPCPSCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSPGWFLDSPDRPWNPPTFSPALLVVTEGDNATFTCSFSNTSESFVL |
| | NWYRMSPSNQTDKLAAFPEDRSQPGQDCRFRVTQLPNGRDFHMSVVRAR |
| | RNDSGTYLCGAISLAPKAQIKESLRAELRVTERRAEVPTAHPSPSPRPAGQF |
| | QTLV |
| SEQ ID NO: 399 | ETVLTQSPGTLSLSPGERATLSCRASQSLGSSYLAWYQQKPGQAPRLLIYGA |
| anti-TGFb LC | SSRAPGIPDRFSGSGSGTDFTLTISRLEPEDFAVYYCQQYADSPITFGQGTRL |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 400 | QVQLVQSGAEVKKPGSSVKVSCKASGYTFSSNVISWVRQAPGQGLEWMG |
| anti-TGFb HC | GVIPIVDIANYAQRFKGRVTITADESTSTTYMELSSLRSEDTAVYYCASTLG |
| fused to | LVLDAMDYWGQGTLVTVSSASTKGPSVFPLAPCSRSTSESTAALGCLVKD |
| SIGLEC10 ECD | YFPEPVTVSWNSGALTSGVHTFPAVLQSSGLYSLSSVVTVPSSSLGTKTYTC |
| | NVDHKPSNTKVDKRVESKYGPPCPSCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSMDGRFWIRVQESVMVPEGLCISVPCSFSYPRQDWTGSTPAYGY |
| | WFKAVTETTKGAPVATNHQSREVEMSTRGRFQLTGDPAKGNCSLVIRDAQ |
| | MQDESQYFFRVERGSYVRYNFMNDGFFLKVTALTQKPDVYIPETLEPGQP |
| | VTVICVFNWAFEECPPPSFSWTGAALSSQGTKPTTSHFSVLSFTPRPQDHNT |
| | DLTCHVDFSRKGVSAQRTVRLRVAYAPRDLVISISRDNTPALEPQPQGNVP |
| | YLEAQKGQFLRLLCAADSQPPATLSWVLQNRVLSSSHPWGPRPLGLELPGV |
| | KAGDSGRYTCRAENRLGSQQRALDLSVQYPPENLRVMVSQANRTVLENL |
| | GNGTSLPVLEGQSLCLVCVTHSSPPARLSWTQRGQVLSPSQPSDPGVLELPR |
| | VQVEHEGEFTCHARHPLGSQHVSLSLSVHYSPKLLGPSCSWEAEGLHCSCS |
| | SQASPAPSLRWWLGEELLEGNSSQDSFEVTPSSAGPWANSSLSLHGGLSSG |
| | LRLRCEAWNVHGAQSGSILQLPDKKGLIST |
| SEQ ID NO: 401 | QVQLVQSGAEVKKPGSSVKVSCKASGYTFSSNVISWVRQAPGQGLEWMG |
| anti-TGFb HC | GVIPIVDIANYAQRFKGRVTITADESTSTTYMELSSLRSEDTAVYYCASTLG |
| fused to SIRPa | LVLDAMDYWGQGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPSCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSEEELQVIQPDKSVLVAAGETATLRCTATSLIPVGPIQWFRGAGPG |
| | RELIYNQKEGHFPRVTTVSDLTKRNNMDFSIRIGNITPADAGTYYCVKFRK |
| | GSPDDVEFKSGAGTELSVRAKPS |
| SEQ ID NO: 402 | QVQLVQSGAEVKKPGSSVKVSCKASGYTFSSNVISWVRQAPGQGLEWMG |
| anti-TGFb HC | GVIPIVDIANYAQRFKGRVTITADESTSTTYMELSSLRSEDTAVYYCASTLG |
| fused to TIM3 | LVLDAMDYWGQGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPSCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSSEVEYRAEVGQNAYLPCFYTPAAPGNLVPVCWGKGACPVFECG |
| | NVVLRTDERDVNYWTSRYWLNGDFRKGDVSLTIENVTLADSGIYCCRIQIP |
| | GIMNDEKFNLKLVIKPAKVTPAPTRQRDFTAAFPRMLTTRGHGPAETQTLG |
| | SLPDINLTQISTLANELRDSRLANDLRDSGATIRIG |
| SEQ ID NO: 403 | QVQLVQSGAEVKKPGSSVKVSCKASGYTFSSNVISWVRQAPGQGLEWMG |
| anti-TGFb HC | GVIPIVDIANYAQRFKGRVTITADESTSTTYMELSSLRSEDTAVYYCASTLG |
| fused to VEGFR | LVLDAMDYWGQGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPSCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSSDTGRPFVEMYSEIPEIIHMTEGRELVIPCRVTSPNITVTLKKFPL |
| | DTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |
| SEQ ID NO: 404 | DVQLQESGPGLVKPSQSLSLTCSVTGYSITSGYYWNWIRQFPGNKLEWMG |
| anti-LAP HC | YISYDGTNNYNPSLKNRISITRDTSKHQFFLKLNSVTTEDTATYYCARSFYN |
| fused to BTLA | NYFDFWGQGTTLTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSKESCDVQLYIKRQSEHSILAGDPFELECPVKYCANRPHVTWCKLN |
| | GTTCVKLEDRQTSWKEEKNISFFILHFEPVLPNDNGSYRCSANFQSNLIESHS |
| | TTLYVTDVKSASERPSKDEMAS |
| SEQ ID NO: 405 | DVQLQESGPGLVKPSQSLSLTCSVTGYSITSGYYWNWIRQFPGNKLEWMG |
| anti-LAP HC | YISYDGTNNYNPSLKNRISITRDTSKHQFFLKLNSVTTEDTATYYCARSFYN |
| fused to PD1 ECD | NYFDFWGQGTTLTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSPGWFLDSPDRPWNPPTFSPALLVVTEGDNATFTCSFSNTSESFVLN |
| | WYRMSPSNQTDKLAAFPEDRSQPGQDCRFRVTQLPNGRDFHMSVVRARR |
| | NDSGTYLCGAISLAPKAQIKESLRAELRVTERRAEVPTAHPSPSPRPAGQFQ |
| | TLV |
| SEQ ID NO: 406 | DVQLQESGPGLVKPSQSLSLTCSVTGYSITSGYYWNWIRQFPGNKLEWMG |
| anti-LAP HC | YISYDGTNNYNPSLKNRISITRDTSKHQFFLKLNSVTTEDTATYYCARSFYN |
| fused to | NYFDFWGQGTTLTVSSASTKGPSVFPLAPSSKSTSGGTAALGCLVKDYFPE |
| SIGLEC10 ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSMDGRFWIRVQESVMVPEGLCISVPCSFSYPRQDWTGSTPAYGYW |
| | FKAVTETTKGAPVATNHQSREVEMSTRGRFQLTGDPAKGNCSLVIRDAQM |
| | QDESQYFFRVERGSYVRYNFMNDGFFLKVTALTQKPDVYIPETLEPGQPVT |
| | VICVFNWAFEECPPPSFSWTGAALSSQGTKPTTSHFSVLSFTPRPQDHNTDL |
| | TCHVDFSRKGVSAQRTVRLRVAYAPRDLVISISRDNTPALEPQPQGNVPYL |
| | EAQKGQFLRLLCAADSQPPATLSWVLQNRVLSSSHPWGPRPLGLELPGVK |
| | AGDSGRYTCRAENRLGSQQRALDLSVQYPPENLRVMVSQANRTVLENLG |
| | NGTSLPVLEGQSLCLVCVTHSSPPARLSWTQRGQVLSPSQPSDPGVLELPRV |
| | QVEHEGEFTCHARHPLGSQHVSLSLSVHYSPKLLGPSCSWEAEGLHCSCSS |
| | QASPAPSLRWWLGEELLEGNSSQDSFEVTPSSAGPWANSSLSLHGGLSSGL |
| | RLRCEAWNVHGAQSGSILQLPDKKGLIST |
| SEQ ID NO: 407 | DVQLQESGPGLVKPSQSLSLTCSVTGYSITSGYYWNWIRQFPGNKLEWMG |
| anti-LAP HC | YISYDGTNNYNPSLKNRISITRDTSKHQFFLKLNSVTTEDTATYYCARSFYN |
| fused to SIRPa | NYFDFWGQGTTLTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSEEELQVIQPDKSVLVAAGETATLRCTATSLIPVGPIQWFRGAGPGR |
| | ELIYNQKEGHFPRVTTVSDLTKRNNMDFSIRIGNITPADAGTYYCVKFRKGS |
| | PDDVEFKSGAGTELSVRAKPS |
| SEQ ID NO: 408 | DVQLQESGPGLVKPSQSLSLTCSVTGYSITSGYYWNWIRQFPGNKLEWMG |
| anti-LAP HC | YISYDGTNNYNPSLKNRISITRDTSKHQFFLKLNSVTTEDTATYYCARSFYN |
| fused to TIM3 | NYFDFWGQGTTLTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSEVEYRAEVGQNAYLPCFYTPAAPGNLVPVCWGKGACPVFECGN |
| | VVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGS |
| | LPDINLTQISTLANELRDSRLANDLRDSGATIRIG |
| SEQ ID NO: 409 | DVQLQESGPGLVKPSQSLSLTCSVTGYSITSGYYWNWIRQFPGNKLEWMG |
| anti-LAP HC | YISYDGTNNYNPSLKNRISITRDTSKHQFFLKLNSVTTEDTATYYCARSFYN |
| fused to VEGFR | NYFDFWGQGTTLTVSSASTKGPSVFPLAPSSKSTSGGTAALGCLVKDYFPE |
| ECD | PVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSDTGRPFVEMYSEIPEIIHMTEGRELVIPCRVTSPNITVTLKKFPLDT |
| | LIPDGKRIIWDSRKGFIISNATYKEIGLLTCEATVNGHLYKTNYLTHRQTNTI |
| | IDVVLSPSHGIELSVGEKLVLNCTARTELNVGIDFNWEYPSSKHQHKKLVN |
| | RDLKTQSGSEMKKFLSTLTIDGVTRSDQGLYTCAASSGLMTKKNSTFVRVH |
| | EK |
| SEQ ID NO: 410 | QVQLVQPGAEVRKPGASVKVSCKASGYRFTSYYIDWVRQAPGQGLEWMG |
| anti-GARP HC | RIDPEDAGTKYAQKFQGRVTMTADTSTSTVYVELSSLRSEDTAVYYCARY |
| fused to BTLA | EWETVVVGDLMYEYEYWGQGTLVTVSSASTKGPSVFPLAPCSRSTSESTA |
| ECD | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGGG |
| | GGSGGGGSGGGGSKESCDVQLYIKRQSEHSILAGDPFELECPVKYCANRPH |
| | VTWCKLNGTTCVKLEDRQTSWKEEKNISFFILHFEPVLPNDNGSYRCSANF |
| | QSNLIESHSTTLYVTDVKSASERPSKDEMAS |
| SEQ ID NO: 411 | QVQLVQPGAEVRKPGASVKVSCKASGYRFTSYYIDWVRQAPGQGLEWMG |
| anti-GARP HC | RIDPEDAGTKYAQKFQGRVTMTADTSTSTVYVELSSLRSEDTAVYYCARY |
| fused to PD1 ECD | EWETVVVGDLMYEYEYWGQGTLVTVSSASTKGPSVFPLAPCSRSTSESTA |
| | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGGG |
| | GGSGGGGSGGGGSPGWFLDSPDRPWNPPTFSPALLVVTEGDNATFTCSFSN |
| | TSESFVLNWYRMSPSNQTDKLAAFPEDRSQPGQDCRFRVTQLPNGRDFHM |
| | SVVRARRNDSGTYLCGAISLAPKAQIKESLRAELRVTERRAEVPTAHPSPSP |
| | RPAGQFQTLV |
| SEQ ID NO: 412 | QVQLVQPGAEVRKPGASVKVSCKASGYRFTSYYIDWVRQAPGQGLEWMG |
| anti-GARP HC | RIDPEDAGTKYAQKFQGRVTMTADTSTSTVYVELSSLRSEDTAVYYCARY |
| fused to | EWETVVVGDLMYEYEYWGQGTLVTVSSASTKGPSVFPLAPCSRSTSESTA |
| SIGLEC10 ECD | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGGG |
| | GGSGGGGSGGGGSMDGRFWIRVQESVMVPEGLCISVPCSFSYPRQDWTGS |
| | TPAYGYWFKAVTETTKGAPVATNHQSREVEMSTRGRFQLTGDPAKGNCSL |
| | VIRDAQMQDESQYFFRVERGSYVRYNFMNDGFFLKVTALTQKPDVYIPET |
| | LEPGQPVTVICVFNWAFEECPPPSFSWTGAALSSQGTKPTTSHFSVLSFTPRP |
| | QDHNTDLTCHVDFSRKGVSAQRTVRLRVAYAPRDLVISISRDNTPALEPQP |
| | QGNVPYLEAQKGQFLRLLCAADSQPPATLSWVLQNRVLSSSHPWGPRPLG |
| | LELPGVKAGDSGRYTCRAENRLGSQQRALDLSVQYPPENLRVMVSQANRT |
| | VLENLGNGTSLPVLEGQSLCLVCVTHSSPPARLSWTQRGQVLSPSQPSDPG |
| | VLELPRVQVEHEGEFTCHARHPLGSQHVSLSLSVHYSPKLLGPSCSWEAEG |
| | LHCSCSSQASPAPSLRWWLGEELLEGNSSQDSFEVTPSSAGPWANSSLSLH |
| | GGLSSGLRLRCEAWNVHGAQSGSILQLPDKKGLIST |
| SEQ ID NO: 413 | QVQLVQPGAEVRKPGASVKVSCKASGYRFTSYYIDWVRQAPGQGLEWMG |
| anti-GARP HC | RIDPEDAGTKYAQKFQGRVTMTADTSTSTVYVELSSLRSEDTAVYYCARY |
| fused to SIRPa | EWETVVVGDLMYEYEYWGQGTLVTVSSASTKGPSVFPLAPCSRSTSESTA |
| ECD | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGGG |
| | GGSGGGGSGGGGSEEELQVIQPDKSVLVAAGETATLRCTATSLIPVGPIQW |
| | FRGAGPGRELIYNQKEGHFPRVTTVSDLTKRNNMDFSIRIGNITPADAGTYY |
| | CVKFRKGSPDDVEFKSGAGTELSVRAKPS |
| SEQ ID NO: 414 | QVQLVQPGAEVRKPGASVKVSCKASGYRFTSYYIDWVRQAPGQGLEWMG |
| anti-GARP HC | RIDPEDAGTKYAQKFQGRVTMTADTSTSTVYVELSSLRSEDTAVYYCARY |
| fused to TIM3 | EWETVVVGDLMYEYEYWGQGTLVTVSSASTKGPSVFPLAPCSRSTSESTA |
| ECD | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGGG |
| | GGSGGGGSGGGGSSEVEYRAEVGQNAYLPCFYTPAAPGNLVPVCWGKGA |
| | CPVFECGNVVLRTDERDVNYWTSRYWLNGDFRKGDVSLTIENVTLADSGI |
| | YCCRIQIPGIMNDEKFNLKLVIKPAKVTPAPTRQRDFTAAFPRMLTTRGHGP |
| | AETQTLGSLPDINLTQISTLANELRDSRLANDLRDSGATIRIG |
| SEQ ID NO: 415 | QVQLVQPGAEVRKPGASVKVSCKASGYRFTSYYIDWVRQAPGQGLEWMG |
| anti-GARP HC | RIDPEDAGTKYAQKFQGRVTMTADTSTSTVYVELSSLRSEDTAVYYCARY |
| fused to VEGFR | EWETVVVGDLMYEYEYWGQGTLVTVSSASTKGPSVFPLAPCSRSTSESTA |
| ECD | ALGCLVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGGG |
| | GGSGGGGSGGGGSSDTGRPFVEMYSEIPEIIHMTEGRELVIPCRVTSPNITVT |
| | LKKFPLDTLIPDGKRIIWDSRKGFIISNATYKEIGLLTCEATVNGHLYKTNYL |
| | THRQTNTIIDVVLSPSHGIELSVGEKLVLNCTARTELNVGIDFNWEYPSSKH |
| | QHKKLVNRDLKTQSGSEMKKFLSTLTIDGVTRSDQGLYTCAASSGLMTKK |
| | NSTFVRVHEK |
| SEQ ID NO: 416 | EVQLVQSGAEVKKPGESLKISCKASGYAFSSSWINWVRQMPGKGLEWMG |
| anti-CD73 HC | RIYPRAGDTNYAGKFKDQVTISADKSISTAYLQWSSLKASDTAMYYCASLL |
| fused to BTLA | DYSMDYWGQGTLVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSKESCDVQLYIKRQSEHSILAGDPFELECPVKYCANRPHVTWCK |
| | LNGTTCVKLEDRQTSWKEEKNISFFILHFEPVLPNDNGSYRCSANFQSNLIE |
| | SHSTTLYVTDVKSASERPSKDEMAS |
| SEQ ID NO: 417 | DIQMTQSPSSLSASVGDRVTITCRASQDISNYLNWYQQKPGKAPKLLIYYTS |
| anti-CD73 LC | RLHSGVPSRFSGSGSGTDFTFTISSLQPEDIATYYCQQGNTLPLTFGQGTKVE |
| fused to PD1 ECD | IKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGECGGGGSGGGGSGGGGSPGWFLDSPDRPWNPPTFSPALLVVTEGD |
| | NATFTCSFSNTSESFVLNWYRMSPSNQTDKLAAFPEDRSQPGQDCRFRVTQ |
| | LPNGRDFHMSVVRARRNDSGTYLCGAISLAPKAQIKESLRAELRVTERRAE |
| | VPTAHPSPSPRPAGQFQTLV |
| SEQ ID NO: 418 | DIQMTQSPSSLSASVGDRVTITCRASQDISNYLNWYQQKPGKAPKLLIYYTS |
| anti-CD73 LC | RLHSGVPSRFSGSGSGTDFTFTISSLQPEDIATYYCQQGNTLPLTFGQGTKVE |
| fused to | IKRTVAAPSVFIFPPSDEQLKSGTASVVCLLNNFYPREAKVQWKVDNALQS |
| SIGLEC10 ECD | GNSQESVTEQDSKDSTYSLSSTLTLSKADYEKHKVYACEVTHQGLSSPVTK |
| | SFNRGECGGGGSGGGGSGGGGSMDGRFWIRVQESVMVPEGLCISVPCSFS |
| | YPRQDWTGSTPAYGYWFKAVTETTKGAPVATNHQSREVEMSTRGRFQLT |
| | GDPAKGNCSLVIRDAQMQDESQYFFRVERGSYVRYNFMNDGFFLKVTALT |
| | QKPDVYIPETLEPGQPVTVICVFNWAFEECPPPSFSWTGAALSSQGTKPTTS |
| | HFSVLSFTPRPQDHNTDLTCHVDFSRKGVSAQRTVRLRVAYAPRDLVISISR |
| | DNTPALEPQPQGNVPYLEAQKGQFLRLLCAADSQPPATLSWVLQNRVLSSS |
| | HPWGPRPLGLELPGVKAGDSGRYTCRAENRLGSQQRALDLSVQYPPENLR |
| | VMVSQANRTVLENLGNGTSLPVLEGQSLCLVCVTHSSPPARLSWTQRGQV |
| | LSPSQPSDPGVLELPRVQVEHEGEFTCHARHPLGSQHVSLSLSVHYSPKLLG |
| | PSCSWEAEGLHCSCSSQASPAPSLRWWLGEELLEGNSSQDSFEVTPSSAGP |
| | WANSSLSLHGGLSSGLRLRCEAWNVHGAQSGSILQLPDKKGLIST |
| SEQ ID NO: 419 | DIQMTQSPSSLSASVGDRVTITCRASQDISNYLNWYQQKPGKAPKLLIYYTS |
| anti-CD73 LC | RLHSGVPSRFSGSGSGTDFTFTISSLQPEDIATYYCQQGNTLPLTFGQGTKVE |
| fused to SIRPa | IKRTVAAPSVFIFPPSDEQLKSGTASVVCLLNNFYPREAKVQWKVDNALQS |
| ECD | GNSQESVTEQDSKDSTYSLSSTLTLSKADYEKHKVYACEVTHQGLSSPVTK |
| | SFNRGECGGGGSGGGGSGGGGSEEELQVIQPDKSVLVAAGETATLRCTATS |
| | LIPVGPIQWFRGAGPGRELIYNQKEGHFPRVTTVSDLTKRNNMDFSIRIGNIT |
| | PADAGTYYCVKFRKGSPDDVEFKSGAGTELSVRAKPS |
| SEQ ID NO: 420 | DIQMTQSPSSLSASVGDRVTITCRASQDISNYLNWYQQKPGKAPKLLIYYTS |
| anti-CD73 LC | RLHSGVPSRFSGSGSGTDFTFTISSLQPEDIATYYCQQGNTLPLTFGQGTKVE |
| fused to TIM3 | IKRTVAAPSVFIFPPSDEQLKSGTASVVCLLNNFYPREAKVQWKVDNALQS |
| ECD | GNSQESVTEQDSKDSTYSLSSTLTLSKADYEKHKVYACEVTHQGLSSPVTK |
| | SFNRGECGGGGSGGGGSGGGGSSEVEYRAEVGQNAYLPCFYTPAAPGNLV |
| | PVCWGKGACPVFECGNVVLRTDERDVNYWTSRYWLNGDFRKGDVSLTIE |
| | NVTLADSGIYCCRIQIPGIMNDEKFNLKLVIKPAKVTPAPTRQRDFTAAFPR |
| | MLTTRGHGPAETQTLGSLPDINLTQISTLANELRDSRLANDLRDSGATIRIG |
| SEQ ID NO: 421 | EVQLVQSGAEVKKPGESLKISCKASGYAFSSSWINWVRQMPGKGLEWMG |
| anti-CD73 HC | RIYPRAGDTNYAGKFKDQVTISADKSISTAYLQWSSLKASDTAMYYCASLL |
| fused to PD1 ECD | DYSMDYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSPGWFLDSPDRPWNPPTFSPALLVVTEGDNATFTCSFSNTSESFV |
| | LNWYRMSPSNQTDKLAAFPEDRSQPGQDCRFRVTQLPNGRDFHMSVVRA |
| | RRNDSGTYLCGAISLAPKAQIKESLRAELRVTERRAEVPTAHPSPSPRPAGQ |
| | FQTLV |
| SEQ ID NO: 422 | DIQMTQSPSSLSASVGDRVTITCRASQDISNYLNWYQQKPGKAPKLLIYYTS |
| anti-CD73 LC | RLHSGVPSRFSGSGSGTDFTFTISSLQPEDIATYYCQQGNTLPLTFGQGTKVE |
| fused to BTLA | IKRTVAAPSVFIFPPSDEQLKSGTASVVCLLNNFYPREAKVQWKVDNALQS |
| ECD | GNSQESVTEQDSKDSTYSLSSTLTLSKADYEKHKVYACEVTHQGLSSPVTK |
| | SFNRGECGGGGSGGGGSGGGGSKESCDVQLYIKRQSEHSILAGDPFELECP |
| | VKYCANRPHVTWCKLNGTTCVKLEDRQTSWKEEKNISFFILHFEPVLPNDN |
| | GSYRCSANFQSNLIESHSTTLYVTDVKSASERPSKDEMAS |
| SEQ ID NO: 423 | EVQLVQSGAEVKKPGESLKISCKASGYAFSSSWINWVRQMPGKGLEWMG |
| anti-CD73 HC | RIYPRAGDTNYAGKFKDQVTISADKSISTAYLQWSSLKASDTAMYYCASLL |
| fused to | DYSMDYWGQGTLVTVSSASTKGPSVFPLAPSSKSTSGGTAALGCLVKDYF |
| SIGLEC10 ECD | PEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSMDGRFWIRVQESVMVPEGLCISVPCSFSYPRQDWTGSTPAYG |
| | YWFKAVTETTKGAPVATNHQSREVEMSTRGRFQLTGDPAKGNCSLVIRDA |
| | QMQDESQYFFRVERGSYVRYNFMNDGFFLKVTALTQKPDVYIPETLEPGQ |
| | PVTVICVFNWAFEECPPPSFSWTGAALSSQGTKPTTSHFSVLSFTPRPQDHN |
| | TDLTCHVDFSRKGVSAQRTVRLRVAYAPRDLVISISRDNTPALEPQPQGNV |
| | PYLEAQKGQFLRLLCAADSQPPATLSWVLQNRVLSSSHPWGPRPLGLELPG |
| | VKAGDSGRYTCRAENRLGSQQRALDLSVQYPPENLRVMVSQANRTVLEN |
| | LGNGTSLPVLEGQSLCLVCVTHSSPPARLSWTQRGQVLSPSQPSDPGVLELP |
| | RVQVEHEGEFTCHARHPLGSQHVSLSLSVHYSPKLLGPSCSWEAEGLHCSC |
| | SSQASPAPSLRWWLGEELLEGNSSQDSFEVTPSSAGPWANSSLSLHGGLSS |
| | GLRLRCEAWNVHGAQSGSILQLPDKKGLIST |
| SEQ ID NO: 424 | EVQLVQSGAEVKKPGESLKISCKASGYAFSSSWINWVRQMPGKGLEWMG |
| anti-CD73 HC | RIYPRAGDTNYAGKFKDQVTISADKSISTAYLQWSSLKASDTAMYYCASLL |
| fused to SIRPa | DYSMDYWGQGTLVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSEEELQVIQPDKSVLVAAGETATLRCTATSLIPVGPIQWFRGAGP |
| | GRELIYNQKEGHFPRVTTVSDLTKRNNMDFSIRIGNITPADAGTYYCVKFR |
| | KGSPDDVEFKSGAGTELSVRAKPS |
| SEQ ID NO: 425 | EVQLVQSGAEVKKPGESLKISCKASGYAFSSSWINWVRQMPGKGLEWMG |
| anti-CD73 HC | RIYPRAGDTNYAGKFKDQVTISADKSISTAYLQWSSLKASDTAMYYCASLL |
| fused to TGFbR | DYSMDYWGQGTLVTVSSASTKGPSVFPLAPSSKSTSGGTAALGCLVKDYF |
| ECD | PEPVTVSWNSGALTSGVHIFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSTIPPHVQKSVNNDMIVTDNNGAVKFPQLCKFCDVRFSTCDNQ |
| | KSCMSNCSITSICEKPQEVCVAVWRKNDENITLETVCHDPKLPYHDFILEDA |
| | ASPKCIMKEKKKPGETFFMCSCSSDECNDNIIFSEEYNTSNPD |
| SEQ ID NO: 426 | EVQLVQSGAEVKKPGESLKISCKASGYAFSSSWINWVRQMPGKGLEWMG |
| anti-CD73 HC | RIYPRAGDTNYAGKFKDQVTISADKSISTAYLQWSSLKASDTAMYYCASLL |
| fused to TIM3 | DYSMDYWGQGTLVTVSSASTKGPSVFPLAPSSKSTSGGTAALGCLVKDYF |
| ECD | PEPVTVSWNSGALTSGVHIFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSSEVEYRAEVGQNAYLPCFYTPAAPGNLVPVCWGKGACPVFEC |
| | GNVVLRTDERDVNYWTSRYWLNGDFRKGDVSLTIENVTLADSGIYCCRIQI |
| | PGIMNDEKFNLKLVIKPAKVTPAPTRQRDFTAAFPRMLTTRGHGPAETQTL |
| | GSLPDINLTQISTLANELRDSRLANDLRDSGATIRIG |
| SEQ ID NO: 427 | EVQLVQSGAEVKKPGESLKISCKASGYAFSSSWINWVRQMPGKGLEWMG |
| anti-CD73 HC | RIYPRAGDTNYAGKFKDQVTISADKSISTAYLQWSSLKASDTAMYYCASLL |
| fused to VEGFR | DYSMDYWGQGTLVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSSDTGRPFVEMYSEIPEIIHMTEGRELVIPCRVTSPNITVTLKKFP |
| | LDTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |
| SEQ ID NO: 428 | QVOLVOSGSELKKPGASVKVSCKASGYTFTHYGMNWVRQAPGQGLKWM |
| anti-CD39 HC | GWINTYTGEPTYADDFKGRFVFSLDTSVSTAYLQISSLKAEDTAVYYCARR |
| fused to BTLA | RYEGNYVFYYFDYWGQGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSKESCDVQLYIKRQSEHSILAGDPFELECPVKYCANRPH |
| | VTWCKLNGTTCVKLEDRQTSWKEEKNISFFILHFEPVLPNDNGSYRCSANF |
| | QSNLIESHSTTLYVTDVKSASERPSKDEMAS |
| SEQ ID NO: 429 | QVOLVOSGSELKKPGASVKVSCKASGYTFTHYGMNWVRQAPGQGLKWM |
| anti-CD39 HC | GWINTYTGEPTYADDFKGRFVFSLDTSVSTAYLQISSLKAEDTAVYYCARR |
| fused to PD1 ECD | RYEGNYVFYYFDYWGQGTTVTVSSASTKGPSVFPLAPSSKSTSGGTAALGC |
| | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSPGWFLDSPDRPWNPPTFSPALLVVTEGDNATFTCSFSN |
| | TSESFVLNWYRMSPSNQTDKLAAFPEDRSQPGQDCRFRVTQLPNGRDFHM |
| | SVVRARRNDSGTYLCGAISLAPKAQIKESLRAELRVTERRAEVPTAHPSPSP |
| | RPAGQFQTLV |
| SEQ ID NO: 430 | QVOLVOSGSELKKPGASVKVSCKASGYTFTHYGMNWVRQAPGQGLKWM |
| anti-CD39 HC | GWINTYTGEPTYADDFKGRFVFSLDTSVSTAYLQISSLKAEDTAVYYCARR |
| fused to | RYEGNYVFYYFDYWGQGTTVTVSSASTKGPSVFPLAPSSKSTSGGTAALGC |
| SIGLEC10 ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSMDGRFWIRVQESVMVPEGLCISVPCSFSYPRQDWTGS |
| | TPAYGYWFKAVTETTKGAPVATNHQSREVEMSTRGRFQLTGDPAKGNCSL |
| | VIRDAQMQDESQYFFRVERGSYVRYNFMNDGFFLKVTALTQKPDVYIPET |
| | LEPGQPVTVICVFNWAFEECPPPSFSWTGAALSSQGTKPTTSHFSVLSFTPRP |
| | QDHNTDLTCHVDFSRKGVSAQRTVRLRVAYAPRDLVISISRDNTPALEPQP |
| | QGNVPYLEAQKGQFLRLLCAADSQPPATLSWVLQNRVLSSSHPWGPRPLG |
| | LELPGVKAGDSGRYTCRAENRLGSQQRALDLSVQYPPENLRVMVSQANRT |
| | VLENLGNGTSLPVLEGQSLCLVCVTHSSPPARLSWTQRGQVLSPSQPSDPG |
| | VLELPRVQVEHEGEFTCHARHPLGSQHVSLSLSVHYSPKLLGPSCSWEAEG |
| | LHCSCSSQASPAPSLRWWLGEELLEGNSSQDSFEVTPSSAGPWANSSLSLH |
| | GGLSSGLRLRCEAWNVHGAQSGSILQLPDKKGLIST |
| SEQ ID NO: 431 | QVOLVOSGSELKKPGASVKVSCKASGYTFTHYGMNWVRQAPGQGLKWM |
| anti-CD39 HC | GWINTYTGEPTYADDFKGRFVFSLDTSVSTAYLQISSLKAEDTAVYYCARR |
| fused to SIRPa | RYEGNYVFYYFDYWGQGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSEEELQVIQPDKSVLVAAGETATLRCTATSLIPVGPIQW |
| | FRGAGPGRELIYNQKEGHFPRVTTVSDLTKRNNMDFSIRIGNITPADAGTYY |
| | CVKFRKGSPDDVEFKSGAGTELSVRAKPS |
| SEQ ID NO: 432 | QVOLVOSGSELKKPGASVKVSCKASGYTFTHYGMNWVRQAPGQGLKWM |
| anti-CD39 HC | GWINTYTGEPTYADDFKGRFVFSLDTSVSTAYLQISSLKAEDTAVYYCARR |
| fused to TGFbR | RYEGNYVFYYFDYWGQGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSTIPPHVQKSVNNDMIVTDNNGAVKFPQLCKFCDVRFS |
| | TCDNQKSCMSNCSITSICEKPQEVCVAVWRKNDENITLETVCHDPKLPYHD |
| | FILEDAASPKCIMKEKKKPGETFFMCSCSSDECNDNIIFSEEYNTSNPD |
| SEQ ID NO: 433 | QVOLVOSGSELKKPGASVKVSCKASGYTFTHYGMNWVRQAPGQGLKWM |
| anti-CD39 HC | GWINTYTGEPTYADDFKGRFVFSLDTSVSTAYLQISSLKAEDTAVYYCARR |
| fused to TIM3 | RYEGNYVFYYFDYWGQGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSSEVEYRAEVGQNAYLPCFYTPAAPGNLVPVCWGKGA |
| | CPVFECGNVVLRTDERDVNYWTSRYWLNGDFRKGDVSLTIENVTLADSGI |
| | YCCRIQIPGIMNDEKFNLKLVIKPAKVTPAPTRQRDFTAAFPRMLTTRGHGP |
| | AETQTLGSLPDINLTQISTLANELRDSRLANDLRDSGATIRIG |
| SEQ ID NO: 434 | QVOLVOSGSELKKPGASVKVSCKASGYTFTHYGMNWVRQAPGQGLKWM |
| anti-CD39 HC | GWINTYTGEPTYADDFKGRFVFSLDTSVSTAYLQISSLKAEDTAVYYCARR |
| fused to VEGFR | RYEGNYVFYYFDYWGQGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSSDTGRPFVEMYSEIPEIIHMTEGRELVIPCRVTSPNITVT |
| | LKKFPLDTLIPDGKRIIWDSRKGFIISNATYKEIGLLTCEATVNGHLYKTNYL |
| | THRQTNTIIDVVLSPSHGIELSVGEKLVLNCTARTELNVGIDFNWEYPSSKH |
| | QHKKLVNRDLKTQSGSEMKKFLSTLTIDGVTRSDQGLYTCAASSGLMTKK |
| | NSTFVRVHEK |
| SEQ ID NO: 435 | QVQLVESGGGVVQPGRSLRLSCAASGFTFSSYTMHWVRQAPGKGLEWVT |
| anti-CTLA4 HC | FISYDGNNKYYADSVKGRFTISRDNSKNTLYLQMNSLRAEDTAIYYCARTG |
| fused to BTLA | WLGPFDYWGQGTLVTVSSASTKGPSVFPLAPSSKSTSGGTAALGCLVKDYF |
| ECD | PEPVTVSWNSGALTSGVHTFYAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGGGGSGGGGSGGGGSKESCDVQLYIKRQSEHSILAG |
| | DPFELECPVKYCANRPHVTWCKLNGTTCVKLEDRQTSWKEEKNISFFILHF |
| | EPVLPNDNGSYRCSANFQSNLIESHSTTLYVTDVKSASERPSKDEMAS |
| SEQ ID NO: 436 | EIVLTQSPGTLSLSPGERATLSCRASQSVGSSYLAWYQQKPGQAPRLLIYGA |
| anti-CTLA4 LC | FSRATGIPDRFSGSGSGTDFTLTISRLEPEDFAVYYCQQYGSSPWTFGQGTK |
| fused to PD1 ECD | VEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPV |
| | TKSFNRGECGGGGSGGGGSGGGGSPGWFLDSPDRPWNPPTFSPALLVVTE |
| | GDNATFTCSFSNTSESFVLNWYRMSPSNQTDKLAAFPEDRSQPGQDCRFRV |
| | TQLPNGRDFHMSVVRARRNDSGTYLCGAISLAPKAQIKESLRAELRVTERR |
| | AEVPTAHPSPSPRPAGQFQTLV |
| SEQ ID NO: 437 | EIVLTQSPGTLSLSPGERATLSCRASQSVGSSYLAWYQQKPGQAPRLLIYGA |
| anti-CTLA4 LC | FSRATGIPDRFSGSGSGTDFTLTISRLEPEDFAVYYCQQYGSSPWTFGQGTK |
| fused to | VEIKRTVAAPSVFIFPPSDEQLKSGTASVVCLLNNFYPREAKVQWKVDNAL |
| SIGLEC10 ECD | QSGNSQESVTEQDSKDSTYSLSSTLTLSKADYEKHKVYACEVTHQGLSSPV |
| | TKSFNRGECGGGGSGGGGSGGGGSMDGRFWIRVQESVMVPEGLCISVPCS |
| | FSYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 438 | EIVLTQSPGTLSLSPGERATLSCRASQSVGSSYLAWYQQKPGQAPRLLIYGA |
| anti-CTLA4 LC | FSRATGIPDRFSGSGSGTDFTLTISRLEPEDFAVYYCQQYGSSPWTFGQGTK |
| fused to SIRPa | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSEEELQVIQPDKSVLVAAGETATLRCT |
| | ATSLIPVGPIQWFRGAGPGRELIYNQKEGHFPRVTTVSDLTKRNNMDFSIRI |
| | GNITPADAGTYYCVKFRKGSPDDVEFKSGAGTELSVRAKPS |
| SEQ ID NO: 439 | EIVLTQSPGTLSLSPGERATLSCRASQSVGSSYLAWYQQKPGQAPRLLIYGA |
| anti-CTLA4 LC | FSRATGIPDRFSGSGSGTDFTLTISRLEPEDFAVYYCQQYGSSPWTFGQGTK |
| fused to TIM3 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSSEVEYRAEVGQNAYLPCFYTPAAPGN |
| | LVPVCWGKGACPVFECGNVVLRTDERDVNYWTSRYWLNGDFRKGDVSL |
| | TIENVTLADSGIYCCRIQIPGIMNDEKFNLKLVIKPAKVTPAPTRQRDFTAAF |
| | PRMLTTRGHGPAETQTLGSLPDINLTQISTLANELRDSRLANDLRDSGATIRI |
| | G |
| SEQ ID NO: 440 | QVQLVESGGGVVQPGRSLRLSCAASGFTFSSYTMHWVRQAPGKGLEWVT |
| anti-CTLA4 HC | FISYDGNNKYYADSVKGRFTISRDNSKNTLYLQMNSLRAEDTAIYYCARTG |
| fused to PD1 ECD | WLGPFDYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGGGGSGGGGSGGGGSPGWFLDSPDRPWNPPTFSPAL |
| | LVVTEGDNATFTCSFSNTSESFVLNWYRMSPSNQTDKLAAFPEDRSQPGQD |
| | CRFRVTQLPNGRDFHMSVVRARRNDSGTYLCGAISLAPKAQIKESLRAELR |
| | VTERRAEVPTAHPSPSPRPAGQFQTLV |
| SEQ ID NO: 441 | EIVLTQSPGTLSLSPGERATLSCRASQSVGSSYLAWYQQKPGQAPRLLIYGA |
| anti-CTLA4 LC | FSRATGIPDRFSGSGSGTDFTLTISRLEPEDFAVYYCQQYGSSPWTFGQGTK |
| fused to BTLA | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSKESCDVQLYIKRQSEHSILAGDPFELE |
| | CPVKYCANRPHVTWCKLNGTTCVKLEDRQTSWKEEKNISFFILHFEPVLPN |
| | DNGSYRCSANFQSNLIESHSTTLYVTDVKSASERPSKDEMAS |
| SEQ ID NO: 442 | QVQLVESGGGVVQPGRSLRLSCAASGFTFSSYTMHWVRQAPGKGLEWVT |
| anti-CTLA4 HC | FISYDGNNKYYADSVKGRFTISRDNSKNTLYLQMNSLRAEDTAIYYCARTG |
| fused to | WLGPFDYWGQGTLVTVSSASTKGPSVFPLAPSSKSTSGGTAALGCLVKDYF |
| SIGLEC10 ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGGGGSGGGGSGGGGSMDGRFWIRVQESVMVPEGLC |
| | ISVPCSFSYPRQDWTGSTPAYGYWFKAVTETTKGAPVATNHQSREVEMST |
| | RGRFQLTGDPAKGNCSLVIRDAQMQDESQYFFRVERGSYVRYNFMNDGFF |
| | LKVTALTQKPDVYIPETLEPGQPVTVICVFNWAFEECPPPSFSWTGAALSSQ |
| | GTKPTTSHFSVLSFTPRPQDHNTDLTCHVDFSRKGVSAQRTVRLRVAYAPR |
| | DLVISISRDNTPALEPQPQGNVPYLEAQKGQFLRLLCAADSQPPATLSWVL |
| | QNRVLSSSHPWGPRPLGLELPGVKAGDSGRYTCRAENRLGSQQRALDLSV |
| | QYPPENLRVMVSQANRTVLENLGNGTSLPVLEGQSLCLVCVTHSSPPARLS |
| | WTQRGQVLSPSQPSDPGVLELPRVQVEHEGEFTCHARHPLGSQHVSLSLSV |
| | HYSPKLLGPSCSWEAEGLHCSCSSQASPAPSLRWWLGEELLEGNSSQDSFE |
| | VTPSSAGPWANSSLSLHGGLSSGLRLRCEAWNVHGAQSGSILQLPDKKGLI |
| | ST |
| SEQ ID NO: 443 | QVQLVESGGGVVQPGRSLRLSCAASGFTFSSYTMHWVRQAPGKGLEWVT |
| anti-CTLA4 HC | FISYDGNNKYYADSVKGRFTISRDNSKNTLYLQMNSLRAEDTAIYYCARTG |
| fused to SIRPa | WLGPFDYWGQGTLVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGGGGSGGGGSGGGGSEEELQVIQPDKSVLVAAGETA |
| | TLRCTATSLIPVGPIQWFRGAGPGRELIYNQKEGHFPRVTTVSDLTKRNNM |
| | DFSIRIGNITPADAGTYYCVKFRKGSPDDVEFKSGAGTELSVRAKPS |
| SEQ ID NO: 444 | QVQLVESGGGVVQPGRSLRLSCAASGFTFSSYTMHWVRQAPGKGLEWVT |
| anti-CTLA4 HC | FISYDGNNKYYADSVKGRFTISRDNSKNTLYLQMNSLRAEDTAIYYCARTG |
| fused to TGFbR | WLGPFDYWGQGTLVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGGGGSGGGGSGGGGSTIPPHVQKSVNNDMIVTDNN |
| | GAVKFPQLCKFCDVRFSTCDNQKSCMSNCSITSICEKPQEVCVAVWRKNDE |
| | NITLETVCHDPKLPYHDFILEDAASPKCIMKEKKKPGETFFMCSCSSDECND |
| | NIIFSEEYNTSNPD |
| SEQ ID NO: 445 | QVQLVESGGGVVQPGRSLRLSCAASGFTFSSYTMHWVRQAPGKGLEWVT |
| anti-CTLA4 HC | FISYDGNNKYYADSVKGRFTISRDNSKNTLYLQMNSLRAEDTAIYYCARTG |
| fused to TIM3 | WLGPFDYWGQGTLVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGGGGSGGGGSGGGGSSEVEYRAEVGQNAYLPCFYT |
| | PAAPGNLVPVCWGKGACPVFECGNVVLRTDERDVNYWTSRYWLNGDFR |
| | KGDVSLTIENVTLADSGIYCCRIQIPGIMNDEKFNLKLVIKPAKVTPAPTRQR |
| | DFTAAFPRMLTTRGHGPAETQTLGSLPDINLTQISTLANELRDSRLANDLRD |
| | SGATIRIG |
| SEQ ID NO: 446 | QVQLVESGGGVVQPGRSLRLSCAASGFTFSSYTMHWVRQAPGKGLEWVT |
| anti-CTLA4 HC | FISYDGNNKYYADSVKGRFTISRDNSKNTLYLQMNSLRAEDTAIYYCARTG |
| fused to VEGFR | WLGPFDYWGQGTLVTVSSASTKGPSVFPLAPSSKSTSGGTAALGCLVKDYF |
| ECD | PEPVTVSWNSGALTSGVHTFPAVLQSSGLYSLSSVVTVPSSSLGTQTYICNV |
| | NHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPS |
| | RDELTKNQVSLTCLVKGFYPSDIAVEWESNGQPENNYKTTPPVLDSDGSFF |
| | LYSKLTVDKSRWQQGGGGSGGGGSGGGGSSDTGRPFVEMYSEIPEIIHMTE |
| | GRELVIPCRVTSPNITVTLKKFPLDTLIPDGKRIIWDSRKGFIISNATYKEIGLL |
| | TCEATVNGHLYKTNYLTHRQTNTIIDVVLSPSHGIELSVGEKLVLNCTARTE |
| | LNVGIDFNWEYPSSKHQHKKLVNRDLKTQSGSEMKKFLSTLTIDGVTRSDQ |
| | GLYTCAASSGLMTKKNSTFVRVHEK |
| SEQ ID NO: 447 | QVQLVQSGVEVKKPGASVKVSCKASGYTFTNYYMYWVRQAPGQGLEWM |
| anti-PD1 HC | GGINPSNGGTNFNEKFKNRVTLTTDSSTTTAYMELKSLQFDDTAVYYCARR |
| fused to BTLA | DYRFDMGFDYWGQGTTVTVSSASTKGPSVFPLAPCSRSTSESTAALGCLVK |
| ECD | DYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSKESCDVQLYIKRQSEHSILAGDPFELECPVKYCANRPHVTWCK |
| | LNGTTCVKLEDRQTSWKEEKNISFFILHEEPVLPNDNGSYRCSANFQSNLIE |
| | SHSTTLYVTDVKSASERPSKDEMAS |
| SEQ ID NO: 448 | EIVLTQSPATLSLSPGERATLSCRASKGVSTSGYSYLHWYQQKPGQAPRLLI |
| anti-PD1 LC | YLASYLESGVPARFSGSGSGTDFTLTISSLEPEDFAVYYCQHSRDLPLTFGG |
| fused to PD1 ECD | GTKVEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQGLS |
| | SPVTKSFNRGECGGGGSGGGGSGGGGSPGWFLDSPDRPWNPPTFSPALLVV |
| | TEGDNATFTCSFSNTSESFVLNWYRMSPSNQTDKLAAFPEDRSQPGQDCRF |
| | RVTQLPNGRDFHMSVVRARRNDSGTYLCGAISLAPKAQIKESLRAELRVTE |
| | RRAEVPTAHPSPSPRPAGQFQTLV |
| SEQ ID NO: 449 | EIVLTQSPATLSLSPGERATLSCRASKGVSTSGYSYLHWYQQKPGQAPRLLI |
| anti-PD1 LC | YLASYLESGVPARFSGSGSGTDFTLTISSLEPEDFAVYYCQHSRDLPLTFGG |
| fused to | GTKVEIKRTVAAPSVFIFPPSDEQLKSGTASVVCLLNNFYPREAKVQWKVD |
| SIGLEC10 ECD | NALQSGNSQESVTEQDSKDSTYSLSSTLTLSKADYEKHKVYACEVTHQGLS |
| | SPVTKSFNRGECGGGGSGGGGSGGGGSMDGRFWIRVQESVMVPEGLCISV |
| | PCSFSYPRQDWTGSTPAYGYWFKAVTETTKGAPVATNHQSREVEMSTRGR |
| | FQLTGDPAKGNCSLVIRDAQMQDESQYFFRVERGSYVRYNFMNDGFFLKV |
| | TALTQKPDVYIPETLEPGQPVTVICVFNWAFEECPPPSFSWTGAALSSQGTK |
| | PTTSHFSVLSFTPRPQDHNTDLTCHVDFSRKGVSAQRTVRLRVAYAPRDLV |
| | ISISRDNTPALEPQPQGNVPYLEAQKGQFLRLLCAADSQPPATLSWVLQNR |
| | VLSSSHPWGPRPLGLELPGVKAGDSGRYTCRAENRLGSQQRALDLSVQYPP |
| | ENLRVMVSQANRTVLENLGNGTSLPVLEGQSLCLVCVTHSSPPARLSWTQ |
| | RGQVLSPSQPSDPGVLELPRVQVEHEGEFTCHARHPLGSQHVSLSLSVHYSP |
| | KLLGPSCSWEAEGLHCSCSSQASPAPSLRWWLGEELLEGNSSQDSFEVTPSS |
| | AGPWANSSLSLHGGLSSGLRLRCEAWNVHGAQSGSILQLPDKKGLIST |
| SEQ ID NO: 450 | EIVLTQSPATLSLSPGERATLSCRASKGVSTSGYSYLHWYQQKPGQAPRLLI |
| anti-PD1 LC | YLASYLESGVPARFSGSGSGTDFTLTISSLEPEDFAVYYCQHSRDLPLTFGG |
| fused to SIRPa | GTKVEIKRTVAAPSVFIFPPSDEQLKSGTASVVCLLNNFYPREAKVQWKVD |
| ECD | NALQSGNSQESVTEQDSKDSTYSLSSTLTLSKADYEKHKVYACEVTHQGLS |
| | SPVTKSFNRGECGGGGSGGGGSGGGGSEEELQVIQPDKSVLVAAGETATLR |
| | CTATSLIPVGPIQWFRGAGPGRELIYNQKEGHFPRVTTVSDLTKRNNMDFSI |
| | RIGNITPADAGTYYCVKFRKGSPDDVEFKSGAGTELSVRAKPS |
| SEQ ID NO: 451 | EIVLTQSPATLSLSPGERATLSCRASKGVSTSGYSYLHWYQQKPGQAPRLLI |
| anti-PD1 LC | YLASYLESGVPARFSGSGSGTDFTLTISSLEPEDFAVYYCQHSRDLPLTFGG |
| fused to TIM3 | GTKVEIKRTVAAPSVFIFPPSDEQLKSGTASVVCLLNNFYPREAKVQWKVD |
| ECD | NALQSGNSQESVTEQDSKDSTYSLSSTLTLSKADYEKHKVYACEVTHQGLS |
| | SPVTKSFNRGECGGGGSGGGGSGGGGSSEVEYRAEVGQNAYLPCFYTPAA |
| | PGNLVPVCWGKGACPVFECGNVVLRTDERDVNYWTSRYWLNGDFRKGD |
| | VSLTIENVTLADSGIYCCRIQIPGIMNDEKFNLKLVIKPAKVTPAPTRQRDFT |
| | AAFPRMLTTRGHGPAETQTLGSLPDINLTQISTLANELRDSRLANDLRDSGA |
| | TIRIG |
| SEQ ID NO: 452 | QVQLVQSGVEVKKPGASVKVSCKASGYTFTNYYMYWVRQAPGQGLEWM |
| anti-PD1 HC | GGINPSNGGTNFNEKFKNRVTLTTDSSTTTAYMELKSLQFDDTAVYYCARR |
| fused to PD1 ECD | DYRFDMGFDYWGQGTTVTVSSASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSPGWFLDSPDRPWNPPTFSPALLVVTEGDNATFTCSFSNTSESFV |
| | LNWYRMSPSNQTDKLAAFPEDRSQPGQDCRFRVTQLPNGRDFHMSVVRA |
| | RRNDSGTYLCGAISLAPKAQIKESLRAELRVTERRAEVPTAHPSPSPRPAGQ |
| | FQTLV |
| SEQ ID NO: 453 | EIVLTQSPATLSLSPGERATLSCRASKGVSTSGYSYLHWYQQKPGQAPRLLI |
| anti-PD1 LC | YLASYLESGVPARFSGSGSGTDFTLTISSLEPEDFAVYYCQHSRDLPLTFGG |
| fused to BTLA | GTKVEIKRTVAAPSVFIFPPSDEQLKSGTASVVCLLNNFYPREAKVQWKVD |
| ECD | NALQSGNSQESVTEQDSKDSTYSLSSTLTLSKADYEKHKVYACEVTHQGLS |
| | SPVTKSFNRGECGGGGSGGGGSGGGGSKESCDVQLYIKRQSEHSILAGDPF |
| | ELECPVKYCANRPHVTWCKLNGTTCVKLEDRQTSWKEEKNISFFILHEEPV |
| | LPNDNGSYRCSANFQSNLIESHSTTLYVTDVKSASERPSKDEMAS |
| SEQ ID NO: 454 | QVQLVQSGVEVKKPGASVKVSCKASGYTFTNYYMYWVRQAPGQGLEWM |
| anti-PD1 HC | GGINPSNGGTNFNEKFKNRVTLTTDSSTTTAYMELKSLQFDDTAVYYCARR |
| fused to | DYRFDMGFDYWGQGTTVTVSSASTKGPSVFPLAPCSRSTSESTAALGCLVK |
| SIGLEC10 ECD | DYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSMDGRFWIRVQESVMVPEGLCISVPCSFSYPRQDWTGSTPAYG |
| | YWFKAVTETTKGAPVATNHQSREVEMSTRGRFQLTGDPAKGNCSLVIRDA |
| | QMQDESQYFFRVERGSYVRYNFMNDGFFLKVTALTQKPDVYIPETLEPGQ |
| | PVTVICVFNWAFEECPPPSFSWTGAALSSQGTKPTTSHFSVLSFTPRPQDHN |
| | TDLTCHVDFSRKGVSAQRTVRLRVAYAPRDLVISISRDNTPALEPQPQGNV |
| | PYLEAQKGQFLRLLCAADSQPPATLSWVLQNRVLSSSHPWGPRPLGLELPG |
| | VKAGDSGRYTCRAENRLGSQQRALDLSVQYPPENLRVMVSQANRTVLEN |
| | LGNGTSLPVLEGQSLCLVCVTHSSPPARLSWTQRGQVLSPSQPSDPGVLELP |
| | RVQVEHEGEFTCHARHPLGSQHVSLSLSVHYSPKLLGPSCSWEAEGLHCSC |
| | SSQASPAPSLRWWLGEELLEGNSSQDSFEVTPSSAGPWANSSLSLHGGLSS |
| | GLRLRCEAWNVHGAQSGSILQLPDKKGLIST |
| SEQ ID NO: 455 | QVQLVQSGVEVKKPGASVKVSCKASGYTFTNYYMYWVRQAPGQGLEWM |
| anti-PD1 HC | GGINPSNGGTNFNEKFKNRVTLTTDSSTTTAYMELKSLQFDDTAVYYCARR |
| fused to SIRPa | DYRFDMGFDYWGQGTTVTVSSASTKGPSVFPLAPCSRSTSESTAALGCLVK |
| ECD | DYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSEEELQVIQPDKSVLVAAGETATLRCTATSLIPVGPIQWFRGAGP |
| | GRELIYNQKEGHFPRVTTVSDLTKRNNMDFSIRIGNITPADAGTYYCVKFR |
| | KGSPDDVEFKSGAGTELSVRAKPS |
| SEQ ID NO: 456 | QVQLVQSGVEVKKPGASVKVSCKASGYTFTNYYMYWVRQAPGQGLEWM |
| anti-PD1 HC | GGINPSNGGTNFNEKFKNRVTLTTDSSTTTAYMELKSLQFDDTAVYYCARR |
| fused to TGFbR | DYRFDMGFDYWGQGTTVTVSSASTKGPSVFPLAPCSRSTSESTAALGCLVK |
| ECD | DYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSTIPPHVQKSVNNDMIVTDNNGAVKFPQLCKFCDVRFSTCDNQ |
| | KSCMSNCSITSICEKPQEVCVAVWRKNDENITLETVCHDPKLPYHDFILEDA |
| | ASPKCIMKEKKKPGETFFMCSCSSDECNDNIIFSEEYNTSNPD |
| SEQ ID NO: 457 | QVQLVQSGVEVKKPGASVKVSCKASGYTFTNYYMYWVRQAPGQGLEWM |
| anti-PD1 HC | GGINPSNGGTNFNEKFKNRVTLTTDSSTTTAYMELKSLQFDDTAVYYCARR |
| fused to TIM3 | DYRFDMGFDYWGQGTTVTVSSASTKGPSVFPLAPCSRSTSESTAALGCLVK |
| ECD | DYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSSEVEYRAEVGQNAYLPCFYTPAAPGNLVPVCWGKGACPVFEC |
| | GNVVLRTDERDVNYWTSRYWLNGDFRKGDVSLTIENVTLADSGIYCCRIQI |
| | PGIMNDEKFNLKLVIKPAKVTPAPTRQRDFTAAFPRMLTTRGHGPAETQTL |
| | GSLPDINLTQISTLANELRDSRLANDLRDSGATIRIG |
| SEQ ID NO: 458 | QVQLVQSGVEVKKPGASVKVSCKASGYTFTNYYMYWVRQAPGQGLEWM |
| anti-PD1 HC | GGINPSNGGTNFNEKFKNRVTLTTDSSTTTAYMELKSLQFDDTAVYYCARR |
| fused to VEGFR | DYRFDMGFDYWGQGTTVTVSSASTKGPSVFPLAPCSRSTSESTAALGCLVK |
| ECD | DYFPEPVTVSWNSGALTSGVHTFPAVLQSSGLYSLSSVVTVPSSSLGTKTYT |
| | CNVDHKPSNTKVDKRVESKYGPPCPPCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSSDTGRPFVEMYSEIPEIIHMTEGRELVIPCRVTSPNITVTLKKFP |
| | LDTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |
| SEQ ID NO: 459 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to BTLA | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSKESCDVQLYIKRQSEHSILAGDPFELECPVKYCANRPHVTWC |
| | KLNGTTCVKLEDRQTSWKEEKNISFFILHFEPVLPNDNGSYRCSANFQSNLI |
| | ESHSTTLYVTDVKSASERPSKDEMAS |
| SEQ ID NO: 460 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to | VEIKRTVAAPSVFIFPPSDEQLKSGTASVVCLLNNFYPREAKVQWKVDNAL |
| SIGLEC10 ECD | QSGNSQESVTEQDSKDSTYSLSSTLTLSKADYEKHKVYACEVTHQGLSSPV |
| | TKSFNRGECGGGGSGGGGSGGGGSMDGRFWIRVQESVMVPEGLCISVPCS |
| | FSYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 461 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to SIRPa | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSEEELQVIQPDKSVLVAAGETATLRCT |
| | ATSLIPVGPIQWFRGAGPGRELIYNQKEGHFPRVTTVSDLTKRNNMDFSIRI |
| | GNITPADAGTYYCVKFRKGSPDDVEFKSGAGTELSVRAKPS |
| SEQ ID NO: 462 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to TIM3 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSSEVEYRAEVGQNAYLPCFYTPAAPGN |
| | LVPVCWGKGACPVFECGNVVLRTDERDVNYWTSRYWLNGDFRKGDVSL |
| | TIENVTLADSGIYCCRIQIPGIMNDEKFNLKLVIKPAKVTPAPTRQRDFTAAF |
| | PRMLTTRGHGPAETQTLGSLPDINLTQISTLANELRDSRLANDLRDSGATIRI |
| | G |
| SEQ ID NO: 463 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to | HWPGGFDYWGQGTLVTVSAASTKGPSVFPLAPSSKSTSGGTAALGCLVKD |
| SIGLEC10 ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSMDGRFWIRVQESVMVPEGLCISVPCSFSYPRQDWTGSTPAY |
| | GYWFKAVTETTKGAPVATNHQSREVEMSTRGRFQLTGDPAKGNCSLVIRD |
| | AQMQDESQYFFRVERGSYVRYNFMNDGFFLKVTALTQKPDVYIPETLEPG |
| | QPVTVICVFNWAFEECPPPSFSWTGAALSSQGTKPTTSHFSVLSFTPRPQDH |
| | NTDLTCHVDFSRKGVSAQRTVRLRVAYAPRDLVISISRDNTPALEPQPQGN |
| | VPYLEAQKGQFLRLLCAADSQPPATLSWVLQNRVLSSSHPWGPRPLGLELP |
| | GVKAGDSGRYTCRAENRLGSQQRALDLSVQYPPENLRVMVSQANRTVLE |
| | NLGNGTSLPVLEGQSLCLVCVTHSSPPARLSWTQRGQVLSPSQPSDPGVLE |
| | LPRVQVEHEGEFTCHARHPLGSQHVSLSLSVHYSPKLLGPSCSWEAEGLHC |
| | SCSSQASPAPSLRWWLGEELLEGNSSQDSFEVTPSSAGPWANSSLSLHGGLS |
| | SGLRLRCEAWNVHGAQSGSILQLPDKKGLIST |
| SEQ ID NO: 464 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to BTLA | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSKESCDVQLYIKRQSEHSILAGDPFELE |
| | CPVKYCANRPHVTWCKLNGTTCVKLEDRQTSWKEEKNISFFILHFEPVLPN |
| | DNGSYRCSANFQSNLIESHSTTLYVTDVKSASERPSKDEMAS |
| SEQ ID NO: 465 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to SIRPa | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSEEELQVIQPDKSVLVAAGETATLRCTATSLIPVGPIQWFRGA |
| | GPGRELIYNQKEGHFPRVTTVSDLTKRNNMDFSIRIGNITPADAGTYYCVKF |
| | RKGSPDDVEFKSGAGTELSVRAKPS |
| SEQ ID NO: 466 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to TGFbR | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSTIPPHVQKSVNNDMIVTDNNGAVKFPQLCKFCDVRFSTCDN |
| | QKSCMSNCSITSICEKPQEVCVAVWRKNDENITLETVCHDPKLPYHDFILED |
| | AASPKCIMKEKKKPGETFFMCSCSSDECNDNIIFSEEYNTSNPD |
| SEQ ID NO: 467 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to TIM3 | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSSEVEYRAEVGQNAYLPCFYTPAAPGNLVPVCWGKGACPVF |
| | ECGNVVLRTDERDVNYWTSRYWLNGDFRKGDVSLTIENVTLADSGIYCCR |
| | IQIPGIMNDEKFNLKLVIKPAKVTPAPTRQRDFTAAFPRMLIIRGHGPAETQ |
| | TLGSLPDINLTQISTLANELRDSRLANDLRDSGATIRIG |
| SEQ ID NO: 468 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to VEGFR | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSSDTGRPFVEMYSEIPEIIHMTEGRELVIPCRVTSPNITVTLKKF |
| | PLDTLIPDGKRIIWDSRKGFIISNATYKEIGLLTCEATVNGHLYKTNYLTHRQ |
| | TNTIIDVVLSPSHGIELSVGEKLVLNCTARTELNVGIDFNWEYPSSKHQHKK |
| | LVNRDLKTQSGSEMKKFLSTLTIDGVTRSDQGLYTCAASSGLMTKKNSTFV |
| | RVHEK |
| SEQ ID NO: 469 | EVQLQQSGPGLVKPSQTLSLTCAISGDSVSSNSAAWNWIRQSPSRGLEWLG |
| anti-TIGIT HC | KTYYRFKWYSDYAVSVKGRITINPDTSKNQFSLQLNSVTPEDTAVFYCTRE |
| fused to BTLA | STTYDLLAGPFDYWGQGTLVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSKESCDVQLYIKRQSEHSILAGDPFELECPVKYCANRPH |
| | VTWCKLNGTTCVKLEDRQTSWKEEKNISFFILHFEPVLPNDNGSYRCSANF |
| | QSNLIESHSTTLYVTDVKSASERPSKDEMAS |
| SEQ ID NO: 470 | DIVMTQSPDSLAVSLGERATINCKSSQTVLYSSNNKKYLAWYQQKPGQPPN |
| anti-TIGIT LC | LLIYWASTRESGVPDRFSGSGSGTDFTLTISSLQAEDVAVYYCQQYYSTPFT |
| fused to PD1 ECD | FGPGTKVEIKRTVAAPSVFIFPPSDEQLKSGTASVVCLLNNFYPREAKVQW |
| | KVDNALQSGNSQESVTEQDSKDSTYSLSSTLTLSKADYEKHKVYACEVTH |
| | QGLSSPVTKSFNRGECGGGGSGGGGSGGGGSPGWFLDSPDRPWNPPTFSPA |
| | LLVVTEGDNATFTCSFSNTSESFVLNWYRMSPSNQTDKLAAFPEDRSQPGQ |
| | DCRFRVTQLPNGRDFHMSVVRARRNDSGTYLCGAISLAPKAQIKESLRAEL |
| | RVTERRAEVPTAHPSPSPRPAGQFQTLV |
| SEQ ID NO: 471 | DIVMTQSPDSLAVSLGERATINCKSSQTVLYSSNNKKYLAWYQQKPGQPPN |
| anti-TIGIT LC | LLIYWASTRESGVPDRFSGSGSGTDFTLTISSLQAEDVAVYYCQQYYSTPFT |
| fused to | FGPGTKVEIKRTVAAPSVFIFPPSDEQLKSGTASVVCLLNNFYPREAKVQW |
| SIGLEC10 ECD | KVDNALQSGNSQESVTEQDSKDSTYSLSSTLTLSKADYEKHKVYACEVTH |
| | QGLSSPVTKSFNRGECGGGGSGGGGSGGGGSMDGRFWIRVQESVMVPEGL |
| | CISVPCSFSYPRQDWTGSTPAYGYWFKAVTETTKGAPVATNHQSREVEMS |
| | TRGRFQLTGDPAKGNCSLVIRDAQMQDESQYFFRVERGSYVRYNFMNDGF |
| | FLKVTALTQKPDVYIPETLEPGQPVTVICVFNWAFEECPPPSFSWTGAALSS |
| | QGTKPTTSHFSVLSFTPRPQDHNTDLTCHVDFSRKGVSAQRTVRLRVAYAP |
| | RDLVISISRDNTPALEPQPQGNVPYLEAQKGQFLRLLCAADSQPPATLSWVL |
| | QNRVLSSSHPWGPRPLGLELPGVKAGDSGRYTCRAENRLGSQQRALDLSV |
| | QYPPENLRVMVSQANRTVLENLGNGTSLPVLEGQSLCLVCVTHSSPPARLS |
| | WTQRGQVLSPSQPSDPGVLELPRVQVEHEGEFTCHARHPLGSQHVSLSLSV |
| | HYSPKLLGPSCSWEAEGLHCSCSSQASPAPSLRWWLGEELLEGNSSQDSEE |
| | VTPSSAGPWANSSLSLHGGLSSGLRLRCEAWNVHGAQSGSILQLPDKKGLI |
| | ST |
| SEQ ID NO: 472 | DIVMTQSPDSLAVSLGERATINCKSSQTVLYSSNNKKYLAWYQQKPGQPPN |
| anti-TIGIT LC | LLIYWASTRESGVPDRFSGSGSGTDFTLTISSLQAEDVAVYYCQQYYSTPFT |
| fused to SIRPa | FGPGTKVEIKRTVAAPSVFIFPPSDEQLKSGTASVVCLLNNFYPREAKVQW |
| ECD | KVDNALQSGNSQESVTEQDSKDSTYSLSSTLTLSKADYEKHKVYACEVTH |
| | QGLSSPVTKSFNRGECGGGGSGGGGSGGGGSEEELQVIQPDKSVLVAAGET |
| | ATLRCTATSLIPVGPIQWFRGAGPGRELIYNQKEGHFPRVTTVSDLTKRNN |
| | MDFSIRIGNITPADAGTYYCVKFRKGSPDDVEFKSGAGTELSVRAKPS |
| SEQ ID NO: 473 | DIVMTQSPDSLAVSLGERATINCKSSQTVLYSSNNKKYLAWYQQKPGQPPN |
| anti-TIGIT LC | LLIYWASTRESGVPDRFSGSGSGTDFTLTISSLQAEDVAVYYCQQYYSTPFT |
| fused to TIM3 | FGPGTKVEIKRTVAAPSVFIFPPSDEQLKSGTASVVCLLNNFYPREAKVQW |
| ECD | KVDNALQSGNSQESVTEQDSKDSTYSLSSTLTLSKADYEKHKVYACEVTH |
| | QGLSSPVTKSFNRGECGGGGSGGGGSGGGGSSEVEYRAEVGQNAYLPCFY |
| | TPAAPGNLVPVCWGKGACPVFECGNVVLRTDERDVNYWTSRYWLNGDFR |
| | KGDVSLTIENVTLADSGIYCCRIQIPGIMNDEKFNLKLVIKPAKVTPAPTRQR |
| | DFTAAFPRMLTTRGHGPAETQTLGSLPDINLTQISTLANELRDSRLANDLRD |
| | SGATIRIG |
| SEQ ID NO: 474 | EVQLQQSGPGLVKPSQTLSLTCAISGDSVSSNSAAWNWIRQSPSRGLEWLG |
| anti-TIGIT HC | KTYYRFKWYSDYAVSVKGRITINPDTSKNQFSLQLNSVTPEDTAVFYCTRE |
| fused to PD1 ECD | STTYDLLAGPFDYWGQGTLVTVSSASTKGPSVFPLAPSSKSTSGGTAALGC |
| | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSPGWFLDSPDRPWNPPTFSPALLVVTEGDNATFTCSFSN |
| | TSESFVLNWYRMSPSNQTDKLAAFPEDRSQPGQDCRFRVTQLPNGRDFHM |
| | SVVRARRNDSGTYLCGAISLAPKAQIKESLRAELRVTERRAEVPTAHPSPSP |
| | RPAGQFQTLV |
| SEQ ID NO: 475 | DIVMTQSPDSLAVSLGERATINCKSSQTVLYSSNNKKYLAWYQQKPGQPPN |
| anti-TIGIT LC | LLIYWASTRESGVPDRFSGSGSGTDFTLTISSLQAEDVAVYYCQQYYSTPFT |
| fused to BTLA | FGPGTKVEIKRTVAAPSVFIFPPSDEQLKSGTASVVCLLNNFYPREAKVQW |
| ECD | KVDNALQSGNSQESVTEQDSKDSTYSLSSTLTLSKADYEKHKVYACEVTH |
| | QGLSSPVTKSFNRGECGGGGSGGGGSGGGGSKESCDVQLYIKRQSEHSILA |
| | GDPFELECPVKYCANRPHVTWCKLNGTTCVKLEDRQTSWKEEKNISFFILH |
| | FEPVLPNDNGSYRCSANFQSNLIESHSTTLYVTDVKSASERPSKDEMAS |
| SEQ ID NO: 476 | EVQLQQSGPGLVKPSQTLSLTCAISGDSVSSNSAAWNWIRQSPSRGLEWLG |
| anti-TIGIT HC | KTYYRFKWYSDYAVSVKGRITINPDTSKNQFSLQLNSVTPEDTAVFYCTRE |
| fused to | STTYDLLAGPFDYWGQGTLVTVSSASTKGPSVFPLAPSSKSTSGGTAALGC |
| SIGLEC10 ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSMDGRFWIRVQESVMVPEGLCISVPCSFSYPRQDWTGS |
| | TPAYGYWFKAVTETTKGAPVATNHQSREVEMSTRGRFQLTGDPAKGNCSL |
| | VIRDAQMQDESQYFFRVERGSYVRYNFMNDGFFLKVTALTQKPDVYIPET |
| | LEPGQPVTVICVFNWAFEECPPPSFSWTGAALSSQGTKPTTSHFSVLSFTPRP |
| | QDHNTDLTCHVDFSRKGVSAQRTVRLRVAYAPRDLVISISRDNTPALEPQP |
| | QGNVPYLEAQKGQFLRLLCAADSQPPATLSWVLQNRVLSSSHPWGPRPLG |
| | LELPGVKAGDSGRYTCRAENRLGSQQRALDLSVQYPPENLRVMVSQANRT |
| | VLENLGNGTSLPVLEGQSLCLVCVTHSSPPARLSWTQRGQVLSPSQPSDPG |
| | VLELPRVQVEHEGEFTCHARHPLGSQHVSLSLSVHYSPKLLGPSCSWEAEG |
| | LHCSCSSQASPAPSLRWWLGEELLEGNSSQDSFEVTPSSAGPWANSSLSLH |
| | GGLSSGLRLRCEAWNVHGAQSGSILQLPDKKGLIST |
| SEQ ID NO: 477 | EVQLQQSGPGLVKPSQTLSLTCAISGDSVSSNSAAWNWIRQSPSRGLEWLG |
| anti-TIGIT HC | KTYYRFKWYSDYAVSVKGRITINPDTSKNQFSLQLNSVTPEDTAVFYCTRE |
| fused to SIRPa | STTYDLLAGPFDYWGQGTLVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSEEELQVIQPDKSVLVAAGETATLRCTATSLIPVGPIQW |
| | FRGAGPGRELIYNQKEGHFPRVTTVSDLTKRNNMDFSIRIGNITPADAGTYY |
| | CVKFRKGSPDDVEFKSGAGTELSVRAKPS |
| SEQ ID NO: 478 | EVQLQQSGPGLVKPSQTLSLTCAISGDSVSSNSAAWNWIRQSPSRGLEWLG |
| anti-TIGIT HC | KTYYRFKWYSDYAVSVKGRITINPDTSKNQFSLQLNSVTPEDTAVFYCTRE |
| fused to TGFbR | STTYDLLAGPFDYWGQGTLVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSTIPPHVQKSVNNDMIVTDNNGAVKFPQLCKFCDVRFS |
| | TCDNQKSCMSNCSITSICEKPQEVCVAVWRKNDENITLETVCHDPKLPYHD |
| | FILEDAASPKCIMKEKKKPGETFFMCSCSSDECNDNIIFSEEYNTSNPD |
| SEQ ID NO: 479 | EVQLQQSGPGLVKPSQTLSLTCAISGDSVSSNSAAWNWIRQSPSRGLEWLG |
| anti-TIGIT HC | KTYYRFKWYSDYAVSVKGRITINPDTSKNQFSLQLNSVTPEDTAVFYCTRE |
| fused to TIM3 | STTYDLLAGPFDYWGQGTLVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSSEVEYRAEVGQNAYLPCFYTPAAPGNLVPVCWGKGA |
| | CPVFECGNVVLRTDERDVNYWTSRYWLNGDFRKGDVSLTIENVTLADSGI |
| | YCCRIQIPGIMNDEKFNLKLVIKPAKVTPAPTRQRDFTAAFPRMLTTRGHGP |
| | AETQTLGSLPDINLTQISTLANELRDSRLANDLRDSGATIRIG |
| SEQ ID NO: 480 | EVQLQQSGPGLVKPSQTLSLTCAISGDSVSSNSAAWNWIRQSPSRGLEWLG |
| anti-TIGIT HC | KTYYRFKWYSDYAVSVKGRITINPDTSKNQFSLQLNSVTPEDTAVFYCTRE |
| fused to VEGFR | STTYDLLAGPFDYWGQGTLVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGKGG |
| | GGSGGGGSGGGGSSDTGRPFVEMYSEIPEIIHMTEGRELVIPCRVTSPNITVT |
| | LKKFPLDTLIPDGKRIIWDSRKGFIISNATYKEIGLLTCEATVNGHLYKTNYL |
| | THRQTNTI1DVVLSPSHGIELSVGEKLVLNCTARTELNVGIDFNWEYPSSKH |
| | QHKKLVNRDLKTQSGSEMKKFLSTLTIDGVTRSDQGLYTCAASSGLMTKK |
| | NSTFVRVHEK |
| SEQ ID NO: 481 | QSALTQPRSVSGSPGQSVTISCTGTSSDVGGYNYVSWYQQHPGKAPKLMIY |
| anti-TIM3 HC | DVSKRPSGVPDRFSGSKSGNTASLTISGLQAEDEADYYCSSYADSVVFGGG |
| fused to BTLA | TKVTVLGQPKAAPSVTLFPPSSEELQANKATLVCLISDFYPGAVTVAWKAD |
| ECD | SSPVKAGVETTTPSKQSNNKYAASSYLSLTPEQWKSHKSYSCQVTHEGSTV |
| | EKTVAPTECSGGGGSGGGGSGGGGSKESCDVQLYIKRQSEHSILAGDPFEL |
| | ECPVKYCANRPHVTWCKLNGTTCVKLEDRQTSWKEEKNISFFILHFEPVLP |
| | NDNGSYRCSANFQSNLIESHSTTLYVTDVKSASERPSKDEMAS |
| SEQ ID NO: 482 | EVQLVESGGGLVQPGGSLRLSCAASGFTFSSYAMSWVRQAPGKGLEWVSA |
| anti-TIM3 LC | ISVSGGSTYYADSVKGRFTISRDNSKNTLYLQMNSLRAEDTAVYYCAKAN |
| fused to PD1 ECD | WGFFDYWGQGTLVTVSSASTKGPSVFPLAPSSKSTSGGTAALGCLVKDYFP |
| | EPVTVSWNSGALTSGVHIFPAVLQSSGLYSLSSVVTVPSSSLGTQTYICNVN |
| | HKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSPGWFLDSPDRPWNPPTFSPALLVVTEGDNATFTCSFSNTSESFVL |
| | NWYRMSPSNQTDKLAAFPEDRSQPGQDCRFRVTQLPNGRDFHMSVVRAR |
| | RNDSGTYLCGAISLAPKAQIKESLRAELRVTERRAEVPTAHPSPSPRPAGQF |
| | QTLV |
| SEQ ID NO: 483 | EVQLVESGGGLVQPGGSLRLSCAASGFTFSSYAMSWVRQAPGKGLEWVSA |
| anti-TIM3 LC | ISVSGGSTYYADSVKGRFTISRDNSKNTLYLQMNSLRAEDTAVYYCAKAN |
| fused to | WGFFDYWGQGTLVTVSSASTKGPSVFPLAPSSKSTSGGTAALGCLVKDYFP |
| SIGLEC10 ECD | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSMDGRFWIRVQESVMVPEGLCISVPCSFSYPRQDWTGSTPAYGY |
| | WFKAVTETTKGAPVATNHQSREVEMSTRGRFQLTGDPAKGNCSLVIRDAQ |
| | MQDESQYFFRVERGSYVRYNFMNDGFFLKVTALTQKPDVYIPETLEPGQP |
| | VTVICVFNWAFEECPPPSFSWTGAALSSQGTKPTTSHFSVLSFTPRPQDHNT |
| | DLTCHVDFSRKGVSAQRTVRLRVAYAPRDLVISISRDNTPALEPQPQGNVP |
| | YLEAQKGQFLRLLCAADSQPPATLSWVLQNRVLSSSHPWGPRPLGLELPGV |
| | KAGDSGRYTCRAENRLGSQQRALDLSVQYPPENLRVMVSQANRTVLENL |
| | GNGTSLPVLEGQSLCLVCVTHSSPPARLSWTQRGQVLSPSQPSDPGVLELPR |
| | VQVEHEGEFTCHARHPLGSQHVSLSLSVHYSPKLLGPSCSWEAEGLHCSCS |
| | SQASPAPSLRWWLGEELLEGNSSQDSFEVTPSSAGPWANSSLSLHGGLSSG |
| | LRLRCEAWNVHGAQSGSILQLPDKKGLIST |
| SEQ ID NO: 484 | EVQLVESGGGLVQPGGSLRLSCAASGFTFSSYAMSWVRQAPGKGLEWVSA |
| anti-TIM3 LC | ISVSGGSTYYADSVKGRFTISRDNSKNTLYLQMNSLRAEDTAVYYCAKAN |
| fused to SIRPa | WGFFDYWGQGTLVTVSSASTKGPSVFPLAPSSKSTSGGTAALGCLVKDYFP |
| ECD | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSEEELQVIQPDKSVLVAAGETATLRCTATSLIPVGPIQWFRGAGPG |
| | RELIYNQKEGHFPRVTTVSDLTKRNNMDFSIRIGNITPADAGTYYCVKFRK |
| | GSPDDVEFKSGAGTELSVRAKPS |
| SEQ ID NO: 485 | EVQLVESGGGLVQPGGSLRLSCAASGFTFSSYAMSWVRQAPGKGLEWVSA |
| anti-TIM3 LC | ISVSGGSTYYADSVKGRFTISRDNSKNTLYLQMNSLRAEDTAVYYCAKAN |
| fused to TIM3 | WGFFDYWGQGTLVTVSSASTKGPSVFPLAPSSKSTSGGTAALGCLVKDYFP |
| ECD | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSSEVEYRAEVGQNAYLPCFYTPAAPGNLVPVCWGKGACPVFECG |
| | NVVLRTDERDVNYWTSRYWLNGDFRKGDVSLTIENVTLADSGIYCCRIQIP |
| | GIMNDEKFNLKLVIKPAKVTPAPTRQRDFTAAFPRMLTTRGHGPAETQTLG |
| | SLPDINLTQISTLANELRDSRLANDLRDSGATIRIG |
| SEQ ID NO: 486 | QSALTQPRSVSGSPGQSVTISCTGTSSDVGGYNYVSWYQQHPGKAPKLMIY |
| anti-TIM3 HC | DVSKRPSGVPDRFSGSKSGNTASLTISGLQAEDEADYYCSSYADSVVFGGG |
| fused to PD1 ECD | TKVTVLGQPKAAPSVTLFPPSSEELQANKATLVCLISDFYPGAVTVAWKAD |
| | SSPVKAGVETTTPSKQSNNKYAASSYLSLTPEQWKSHKSYSCQVTHEGSTV |
| | EKTVAPTECSGGGGSGGGGSGGGGSPGWFLDSPDRPWNPPTFSPALLVVTE |
| | GDNATFTCSFSNTSESFVLNWYRMSPSNQTDKLAAFPEDRSQPGQDCRFRV |
| | TQLPNGRDFHMSVVRARRNDSGTYLCGAISLAPKAQIKESLRAELRVTERR |
| | AEVPTAHPSPSPRPAGQFQTLV |
| SEQ ID NO: 487 | EVQLVESGGGLVQPGGSLRLSCAASGFTFSSYAMSWVRQAPGKGLEWVSA |
| anti-TIM3 LC | ISVSGGSTYYADSVKGRFTISRDNSKNTLYLQMNSLRAEDTAVYYCAKAN |
| fused to BTLA | WGFFDYWGQGTLVTVSSASTKGPSVFPLAPSSKSTSGGTAALGCLVKDYFP |
| ECD | EPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSKESCDVQLYIKRQSEHSILAGDPFELECPVKYCANRPHVTWCKL |
| | NGTTCVKLEDRQTSWKEEKNISFFILHEEPVLPNDNGSYRCSANFQSNLIES |
| | HSTTLYVTDVKSASERPSKDEMAS |
| SEQ ID NO: 488 | QSALTQPRSVSGSPGQSVTISCTGTSSDVGGYNYVSWYQQHPGKAPKLMIY |
| anti-TIM3 HC | DVSKRPSGVPDRFSGSKSGNTASLTISGLQAEDEADYYCSSYADSVVFGGG |
| fused to | TKVTVLGQPKAAPSVTLFPPSSEELQANKATLVCLISDFYPGAVTVAWKAD |
| SIGLEC10 ECD | SSPVKAGVETTTPSKQSNNKYAASSYLSLTPEQWKSHKSYSCQVTHEGSTV |
| | EKTVAPTECSGGGGSGGGGSGGGGSMDGRFWIRVQESVMVPEGLCISVPC |
| | SFSYPRQDWTGSTPAYGYWFKAVTETTKGAPVATNHQSREVEMSTRGRFQ |
| | LTGDPAKGNCSLVIRDAQMQDESQYFFRVERGSYVRYNFMNDGFFLKVTA |
| | LTQKPDVYIPETLEPGQPVTVICVFNWAFEECPPPSFSWTGAALSSQGTKPT |
| | TSHFSVLSFTPRPQDHNTDLTCHVDFSRKGVSAQRTVRLRVAYAPRDLVISI |
| | SRDNTPALEPQPQGNVPYLEAQKGQFLRLLCAADSQPPATLSWVLQNRVL |
| | SSSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 489 | QSALTQPRSVSGSPGQSVTISCTGTSSDVGGYNYVSWYQQHPGKAPKLMIY |
| anti-TIM3 HC | DVSKRPSGVPDRFSGSKSGNTASLTISGLQAEDEADYYCSSYADSVVFGGG |
| fused to SIRPa | TKVTVLGQPKAAPSVTLFPPSSEELQANKATLVCLISDFYPGAVTVAWKAD |
| ECD | SSPVKAGVETTTPSKQSNNKYAASSYLSLTPEQWKSHKSYSCQVTHEGSTV |
| | EKTVAPTECSGGGGSGGGGSGGGGSEEELQVIQPDKSVLVAAGETATLRCT |
| | ATSLIPVGPIQWFRGAGPGRELIYNQKEGHFPRVTTVSDLTKRNNMDFSIRI |
| | GNITPADAGTYYCVKFRKGSPDDVEFKSGAGTELSVRAKPS |
| SEQ ID NO: 490 | QSALTQPRSVSGSPGQSVTISCTGTSSDVGGYNYVSWYQQHPGKAPKLMIY |
| anti-TIM3 HC | DVSKRPSGVPDRFSGSKSGNTASLTISGLQAEDEADYYCSSYADSVVFGGG |
| fused to TGFbR | TKVTVLGQPKAAPSVTLFPPSSEELQANKATLVCLISDFYPGAVTVAWKAD |
| ECD | SSPVKAGVETTTPSKQSNNKYAASSYLSLTPEQWKSHKSYSCQVTHEGSTV |
| | EKTVAPTECSGGGGSGGGGSGGGGSTIPPHVQKSVNNDMIVTDNNGAVKF |
| | PQLCKFCDVRFSTCDNQKSCMSNCSITSICEKPQEVCVAVWRKNDENITLE |
| | TVCHDPKLPYHDFILEDAASPKCIMKEKKKPGETFFMCSCSSDECNDNIIFSE |
| | EYNTSNPD |
| SEQ ID NO: 491 | QSALTQPRSVSGSPGQSVTISCTGTSSDVGGYNYVSWYQQHPGKAPKLMIY |
| anti-TIM3 HC | DVSKRPSGVPDRFSGSKSGNTASLTISGLQAEDEADYYCSSYADSVVFGGG |
| fused to TIM3 | TKVTVLGQPKAAPSVTLFPPSSEELQANKATLVCLISDFYPGAVTVAWKAD |
| ECD | SSPVKAGVETTTPSKQSNNKYAASSYLSLTPEQWKSHKSYSCQVTHEGSTV |
| | EKTVAPTECSGGGGSGGGGSGGGGSSEVEYRAEVGQNAYLPCFYTPAAPG |
| | NLVPVCWGKGACPVFECGNVVLRTDERDVNYWTSRYWLNGDFRKGDVS |
| | LTIENVTLADSGIYCCRIQIPGIMNDEKFNLKLVIKPAKVTPAPTRQRDFTAA |
| | FPRMLTTRGHGPAETQTLGSLPDINLTQISTLANELRDSRLANDLRDSGATI |
| | RIG |
| SEQ ID NO: 492 | QSALTQPRSVSGSPGQSVTISCTGTSSDVGGYNYVSWYQQHPGKAPKLMIY |
| anti-TIM3 HC | DVSKRPSGVPDRFSGSKSGNTASLTISGLQAEDEADYYCSSYADSVVFGGG |
| fused to VEGFR | TKVTVLGQPKAAPSVTLFPPSSEELQANKATLVCLISDFYPGAVTVAWKAD |
| ECD | SSPVKAGVETTTPSKQSNNKYAASSYLSLTPEQWKSHKSYSCQVTHEGSTV |
| | EKTVAPTECSGGGGSGGGGSGGGGSSDTGRPFVEMYSEIPEIIHMTEGRELV |
| | IPCRVTSPNITVTLKKFPLDTLIPDGKRIIWDSRKGFIISNATYKEIGLLTCEAT |
| | VNGHLYKTNYLTHRQTNTIIDVVLSPSHGIELSVGEKLVLNCTARTELNVGI |
| | DFNWEYPSSKHQHKKLVNRDLKTQSGSEMKKFLSTLTIDGVTRSDQGLYT |
| | CAASSGLMTKKNSTFVRVHEK |
| SEQ ID NO: 493 | QVQLQQWGAGLLKPSETLSLTCAVYGGSFSGYYWSWIRQSPEKGLEWIGEI |
| anti-41BB HC | NHGGYVTYNPSLESRVTISVDTSKNQFSLKLSSVTAADTAVYYCARDYGPG |
| fused to BTLA | NYDWYFDLWGRGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSKESCDVQLYIKRQSEHSILAGDPFELECPVKYCANRPHVTWCKL |
| | NGTTCVKLEDRQTSWKEEKNISFFILHEEPVLPNDNGSYRCSANFQSNLIES |
| | HSTTLYVTDVKSASERPSKDEMAS |
| SEQ ID NO: 494 | EIVLTQSPATLSLSPGERATLSCRASQSVSSYLAWYQQKPGQAPRLLIYDAS |
| anti-41BB LC | NRATGIPARFSGSGSGTDFTLTISSLEPEDFAVYYCQQRSNWPPALTFCGGT |
| fused to PD1 ECD | KVEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSSP |
| | VTKSFNRGECGGGGSGGGGSGGGGSPGWFLDSPDRPWNPPTFSPALLVVT |
| | EGDNATFTCSFSNTSESFVLNWYRMSPSNQTDKLAAFPEDRSQPGQDCRFR |
| | VTQLPNGRDFHMSVVRARRNDSGTYLCGAISLAPKAQIKESLRAELRVTER |
| | RAEVPTAHPSPSPRPAGQFQTLV |
| SEQ ID NO: 495 | EIVLTQSPATLSLSPGERATLSCRASQSVSSYLAWYQQKPGQAPRLLIYDAS |
| anti-41BB LC | NRATGIPARFSGSGSGTDFTLTISSLEPEDFAVYYCQQRSNWPPALTFCGGT |
| fused to | KVEIKRTVAAPSVFIFPPSDEQLKSGTASVVCLLNNFYPREAKVQWKVDNA |
| SIGLEC10 ECD | LQSGNSQESVTEQDSKDSTYSLSSTLTLSKADYEKHKVYACEVTHQGLSSP |
| | VTKSFNRGECGGGGSGGGGSGGGGSMDGRFWIRVQESVMVPEGLCISVPC |
| | SFSYPRQDWTGSTPAYGYWFKAVTETTKGAPVATNHQSREVEMSTRGRFQ |
| | LTGDPAKGNCSLVIRDAQMQDESQYFFRVERGSYVRYNFMNDGFFLKVTA |
| | LTQKPDVYIPETLEPGQPVTVICVFNWAFEECPPPSFSWTGAALSSQGTKPT |
| | TSHFSVLSFTPRPQDHNTDLTCHVDFSRKGVSAQRTVRLRVAYAPRDLVISI |
| | SRDNTPALEPQPQGNVPYLEAQKGQFLRLLCAADSQPPATLSWVLQNRVL |
| | SSSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 496 | EIVLTQSPATLSLSPGERATLSCRASQSVSSYLAWYQQKPGQAPRLLIYDAS |
| anti-41BB LC | NRATGIPARFSGSGSGTDFTLTISSLEPEDFAVYYCQQRSNWPPALTFCGGT |
| fused to SIRPa | KVEIKRTVAAPSVFIFPPSDEQLKSGTASVVCLLNNFYPREAKVQWKVDNA |
| ECD | LQSGNSQESVTEQDSKDSTYSLSSTLTLSKADYEKHKVYACEVTHQGLSSP |
| | VTKSFNRGECGGGGSGGGGSGGGGSEEELQVIQPDKSVLVAAGETATLRC |
| | TATSLIPVGPIQWFRGAGPGRELIYNQKEGHFPRVTTVSDLTKRNNMDFSIR |
| | IGNITPADAGTYYCVKFRKGSPDDVEFKSGAGTELSVRAKPS |
| SEQ ID NO: 497 | EIVLTQSPATLSLSPGERATLSCRASQSVSSYLAWYQQKPGQAPRLLIYDAS |
| anti-41BB LC | NRATGIPARFSGSGSGTDFTLTISSLEPEDFAVYYCQQRSNWPPALTFCGGT |
| fused to TIM3 | KVEIKRTVAAPSVFIFPPSDEQLKSGTASVVCLLNNFYPREAKVQWKVDNA |
| ECD | LQSGNSQESVTEQDSKDSTYSLSSTLTLSKADYEKHKVYACEVTHQGLSSP |
| | VTKSFNRGECGGGGSGGGGSGGGGSSEVEYRAEVGQNAYLPCFYTPAAPG |
| | NLVPVCWGKGACPVFECGNVVLRTDERDVNYWTSRYWLNGDFRKGDVS |
| | LTIENVTLADSGIYCCRIQIPGIMNDEKFNLKLVIKPAKVTPAPTRQRDFTAA |
| | FPRMLTTRGHGPAETQTLGSLPDINLTQISTLANELRDSRLANDLRDSGATT |
| | RIG |
| SEQ ID NO: 498 | QVQLQQWGAGLLKPSETLSLTCAVYGGSFSGYYWSWIRQSPEKGLEWIGEI |
| anti-41BB HC | NHGGYVTYNPSLESRVTISVDTSKNQFSLKLSSVTAADTAVYYCARDYGPG |
| fused to PD1 ECD | NYDWYFDLWGRGTLVTVSSASTKGPSVFPLAPCSRSTSESTAALGCLVKD |
| | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSPGWFLDSPDRPWNPPTFSPALLVVTEGDNATFTCSFSNTSESFVL |
| | NWYRMSPSNQTDKLAAFPEDRSQPGQDCRFRVTQLPNGRDFHMSVVRAR |
| | RNDSGTYLCGAISLAPKAQIKESLRAELRVTERRAEVPTAHPSPSPRPAGQF |
| | QTLV |
| SEQ ID NO: 499 | EIVLTQSPATLSLSPGERATLSCRASQSVSSYLAWYQQKPGQAPRLLIYDAS |
| anti-41BB LC | NRATGIPARFSGSGSGTDFTLTISSLEPEDFAVYYCQQRSNWPPALTFCGGT |
| fused to BTLA | KVEIKRTVAAPSVFIFPPSDEQLKSGTASVVCLLNNFYPREAKVQWKVDNA |
| ECD | LQSGNSQESVTEQDSKDSTYSLSSTLTLSKADYEKHKVYACEVTHQGLSSP |
| | VTKSFNRGECGGGGSGGGGSGGGGSKESCDVQLYIKRQSEHSILAGDPFEL |
| | ECPVKYCANRPHVTWCKLNGTTCVKLEDRQTSWKEEKNISFFILHFEPVLP |
| | NDNGSYRCSANFQSNLIESHSTTLYVTDVKSASERPSKDEMAS |
| SEQ ID NO: 500 | QVQLQQWGAGLLKPSETLSLTCAVYGGSFSGYYWSWIRQSPEKGLEWIGEI |
| anti-41BB HC | NHGGYVTYNPSLESRVTISVDTSKNQFSLKLSSVTAADTAVYYCARDYGPG |
| fused to | NYDWYFDLWGRGTLVTVSSASTKGPSVFPLAPCSRSTSESTAALGCLVKD |
| SIGLEC10 ECD | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSMDGRFWIRVQESVMVPEGLCISVPCSFSYPRQDWTGSTPAYGY |
| | WFKAVTETTKGAPVATNHQSREVEMSTRGRFQLTGDPAKGNCSLVIRDAQ |
| | MQDESQYFFRVERGSYVRYNFMNDGFFLKVTALTQKPDVYIPETLEPGQP |
| | VTVICVFNWAFEECPPPSFSWTGAALSSQGTKPTTSHFSVLSFTPRPQDHNT |
| | DLTCHVDFSRKGVSAQRTVRLRVAYAPRDLVISISRDNTPALEPQPQGNVP |
| | YLEAQKGQFLRLLCAADSQPPATLSWVLQNRVLSSSHPWGPRPLGLELPGV |
| | KAGDSGRYTCRAENRLGSQQRALDLSVQYPPENLRVMVSQANRTVLENL |
| | GNGTSLPVLEGQSLCLVCVTHSSPPARLSWTQRGQVLSPSQPSDPGVLELPR |
| | VQVEHEGEFTCHARHPLGSQHVSLSLSVHYSPKLLGPSCSWEAEGLHCSCS |
| | SQASPAPSLRWWLGEELLEGNSSQDSFEVTPSSAGPWANSSLSLHGGLSSG |
| | LRLRCEAWNVHGAQSGSILQLPDKKGLIST |
| SEQ ID NO: 501 | QVQLQQWGAGLLKPSETLSLTCAVYGGSFSGYYWSWIRQSPEKGLEWIGEI |
| anti-41BB HC | NHGGYVTYNPSLESRVTISVDTSKNQFSLKLSSVTAADTAVYYCARDYGPG |
| fused to SIRPa | NYDWYFDLWGRGTLVTVSSASTKGPSVFPLAPCSRSTSESTAALGCLVKD |
| EC | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSEEELQVIQPDKSVLVAAGETATLRCTATSLIPVGPIQWFRGAGPG |
| | RELIYNQKEGHFPRVTTVSDLTKRNNMDFSIRIGNITPADAGTYYCVKFRK |
| | GSPDDVEFKSGAGTELSVRAKPS |
| SEQ ID NO: 502 | QVQLQQWGAGLLKPSETLSLTCAVYGGSFSGYYWSWIRQSPEKGLEWIGEI |
| anti-41BB HC | NHGGYVTYNPSLESRVTISVDTSKNQFSLKLSSVTAADTAVYYCARDYGPG |
| fused to TGFbR | NYDWYFDLWGRGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSTIPPHVQKSVNNDMIVTDNNGAVKFPQLCKFCDVRFSTCDNQK |
| | SCMSNCSITSICEKPQEVCVAVWRKNDENITLETVCHDPKLPYHDFILEDAA |
| | SPKCIMKEKKKPGETFFMCSCSSDECNDNIIFSEEYNTSNPD |
| SEQ ID NO: 503 | QVQLQQWGAGLLKPSETLSLTCAVYGGSFSGYYWSWIRQSPEKGLEWIGEI |
| anti-41BB HC | NHGGYVTYNPSLESRVTISVDTSKNQFSLKLSSVTAADTAVYYCARDYGPG |
| fused to TIM3 | NYDWYFDLWGRGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSSEVEYRAEVGQNAYLPCFYTPAAPGNLVPVCWGKGACPVFECG |
| | NVVLRTDERDVNYWTSRYWLNGDFRKGDVSLTIENVTLADSGIYCCRIQIP |
| | GIMNDEKFNLKLVIKPAKVTPAPTRQRDFTAAFPRMLTTRGHGPAETQTLG |
| | SLPDINLTQISTLANELRDSRLANDLRDSGATIRIG |
| SEQ ID NO: 504 | QVQLQQWGAGLLKPSETLSLTCAVYGGSFSGYYWSWIRQSPEKGLEWIGEI |
| anti-41BB HC | NHGGYVTYNPSLESRVTISVDTSKNQFSLKLSSVTAADTAVYYCARDYGPG |
| fused to VEGFR | NYDWYFDLWGRGTLVTVSSASTKGPSVFPLAPCSRSTSESTAALGCLVKD |
| ECD | YFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISR |
| | TPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQ |
| | EEMTKNQVSLTCLVKGFYPSDIAVEWESNGQPENNYKTTPPVLDSDGSFFL |
| | YSRLTVDKSRWQEGNVFSCSVMHEALHNHYTQKSLSLSLGKGGGGSGGG |
| | GSGGGGSSDTGRPFVEMYSEIPEIIHMTEGRELVIPCRVTSPNITVTLKKFPL |
| | DTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |
| SEQ ID NO: 505 | QVQLVQSGSELKKPGASVKVSCKASGYTFTDYSMHWVRQAPGQGLKWM |
| anti-OX40 HC | GWINTETGEPTYADDFKGRFVFSLDTSVSTAYLQISSLKAEDTAVYYCANP |
| fused to BTLA | YYDYVSYYAMDYWGOGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKFIPPV |
| | LDSDGSFFLYSKLTVDKSRWQQGNVFSCSVMHEALHNHYTQKSLSLSPGK |
| | GGGGSGGGGSGGGGSKESCDVQLYIKRQSEHSILAGDPFELECPVKYCANR |
| | PHVTWCKLNGTTCVKLEDRQTSWKEEKNISFFILHEEPVLPNDNGSYRCSA |
| | NFQSNLIESHSTTLYVTDVKSASERPSKDEMAS |
| SEQ ID NO: 506 | DIQMTQSPSSLSASVGDRVTITCKASQDVSTAVAWYQQKPGKAPKLLIYSA |
| anti-OX40 LC | SYLYTGVPSRFSGSGSGTDFTFTISSLQPEDIATYYCOQHYSTPRTFGQGTKL |
| fused to PD1 ECD | EIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVT |
| | KSFNRGECGGGGSGGGGSGGGGSPGWFLDSPDRPWNPPTFSPALLVVTEG |
| | DNATFTCSFSNTSESFVLNWYRMSPSNQTDKLAAFPEDRSQPGQDCRFRVT |
| | QLPNGRDFHMSVVRARRNDSGTYLCGAISLAPKAQIKESLRAELRVTERRA |
| | EVPTAHPSPSPRPAGQFQTLV |
| SEQ ID NO: 507 | DIQMTQSPSSLSASVGDRVTITCKASQDVSTAVAWYQQKPGKAPKLLIYSA |
| anti-OX40 LC | SYLYTGVPSRFSGSGSGTDFTFTISSLQPEDIATYYCOQHYSTPRTFGQGTKL |
| fused to | EIKRTVAAPSVFIFPPSDEQLKSGTASVVCLLNNFYPREAKVQWKVDNALQ |
| SIGLEC10 ECD | SGNSQESVTEQDSKDSTYSLSSTLTLSKADYEKHKVYACEVTHQGLSSPVT |
| | KSFNRGECGGGGSGGGGSGGGGSMDGRFWIRVQESVMVPEGLCISVPCSF |
| | SYPRQDWTGSTPAYGYWFKAVTETTKGAPVATNHQSREVEMSTRGRFQL |
| | TGDPAKGNCSLVIRDAQMQDESQYFFRVERGSYVRYNFMNDGFFLKVTAL |
| | TQKPDVYIPETLEPGQPVTVICVFNWAFEECPPPSFSWTGAALSSQGTKPTT |
| | SHFSVLSFTPRPQDHNTDLTCHVDFSRKGVSAQRTVRLRVAYAPRDLVISIS |
| | RDNTPALEPQPQGNVPYLEAQKGQFLRLLCAADSQPPATLSWVLQNRVLS |
| | SSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 508 | DIQMTQSPSSLSASVGDRVTITCKASQDVSTAVAWYQQKPGKAPKLLIYSA |
| anti-OX40 LC | SYLYTGVPSRFSGSGSGTDFTFTISSLQPEDIATYYCOQHYSTPRTFGQGTKL |
| fused to SIRPa | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSEEELQVIQPDKSVLVAAGETATLRCTA |
| | TSLIPVGPIQWFRGAGPGRELIYNQKEGHFPRVTTVSDLTKRNNMDFSIRIG |
| | NITPADAGTYYCVKFRKGSPDDVEFKSGAGTELSVRAKPS |
| SEQ ID NO: 509 | DIQMTQSPSSLSASVGDRVTITCKASQDVSTAVAWYQQKPGKAPKLLIYSA |
| anti-OX40 LC | SYLYTGVPSRFSGSGSGTDFTFTISSLQPEDIATYYCOQHYSTPRTFGQGTKL |
| fused to TIM3 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSSEVEYRAEVGQNAYLPCFYTPAAPGNL |
| | VPVCWGKGACPVFECGNVVLRTDERDVNYWTSRYWLNGDFRKGDVSLTI |
| | ENVTLADSGIYCCRIQIPGIMNDEKFNLKLVIKPAKVTPAPTRQRDFTAAFP |
| | RMLTTRGHGPAETQTLGSLPDINLTQISTLANELRDSRLANDLRDSGATIRI |
| | G |
| SEQ ID NO: 510 | QVQLVQSGSELKKPGASVKVSCKASGYTFTDYSMHWVRQAPGQGLKWM |
| anti-OX40 HC | GWINTETGEPTYADDFKGRFVFSLDTSVSTAYLQISSLKAEDTAVYYCANP |
| fused to PD1 ECD | YYDYVSYYAMDYWGOGTTVTVSSASTKGPSVFPLAPSSKSTSGGTAALGC |
| | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSPGWFLDSPDRPWNPPTFSPALLVVTEGDNATFTCSF |
| | SNTSESFVLNWYRMSPSNQTDKLAAFPEDRSQPGQDCRFRVTQLPNGRDF |
| | HMSVVRARRNDSGTYLCGAISLAPKAQIKESLRAELRVTERRAEVPTAHPS |
| | PSPRPAGQFQTLV |
| SEQ ID NO: 511 | DIQMTQSPSSLSASVGDRVTITCKASQDVSTAVAWYQQKPGKAPKLLIYSA |
| anti-OX40 LC | SYLYTGVPSRFSGSGSGTDFTFTISSLQPEDIATYYCOQHYSTPRTFGQGTKL |
| fused to BTLA | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSKESCDVQLYIKRQSEHSILAGDPFELEC |
| | PVKYCANRPHVTWCKLNGTTCVKLEDRQTSWKEEKNISFFILHFEPVLPND |
| | NGSYRCSANFQSNLIESHSTTLYVTDVKSASERPSKDEMAS |
| SEQ ID NO: 512 | QVQLVQSGSELKKPGASVKVSCKASGYTFTDYSMHWVRQAPGQGLKWM |
| anti-OX40 HC | GWINTETGEPTYADDFKGRFVFSLDTSVSTAYLQISSLKAEDTAVYYCANP |
| fused to | YYDYVSYYAMDYWGOGTTVTVSSASTKGPSVFPLAPSSKSTSGGTAALGC |
| SIGLEC10 ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSMDGRFWIRVQESVMVPEGLCISVPCSFSYPRQDWT |
| | GSTPAYGYWFKAVTETTKGAPVATNHQSREVEMSTRGRFQLTGDPAKGN |
| | CSLVIRDAQMQDESQYFFRVERGSYVRYNFMNDGFFLKVTALTQKPDVYI |
| | PETLEPGQPVTVICVFNWAFEECPPPSFSWTGAALSSQGTKPTTSHFSVLSFT |
| | PRPQDHNTDLTCHVDFSRKGVSAQRTVRLRVAYAPRDLVISISRDNTPALE |
| | PQPQGNVPYLEAQKGQFLRLLCAADSQPPATLSWVLQNRVLSSSHPWGPR |
| | PLGLELPGVKAGDSGRYTCRAENRLGSQQRALDLSVQYPPENLRVMVSQA |
| | NRTVLENLGNGTSLPVLEGQSLCLVCVTHSSPPARLSWTQRGQVLSPSQPS |
| | DPGVLELPRVQVEHEGEFTCHARHPLGSQHVSLSLSVHYSPKLLGPSCSWE |
| | AEGLHCSCSSQASPAPSLRWWLGEELLEGNSSQDSFEVTPSSAGPWANSSL |
| | SLHGGLSSGLRLRCEAWNVHGAQSGSILQLPDKKGLIST |
| SEQ ID NO: 513 | QVQLVQSGSELKKPGASVKVSCKASGYTFTDYSMHWVRQAPGQGLKWM |
| anti-OX40 HC | GWINTETGEPTYADDFKGRFVFSLDTSVSTAYLQISSLKAEDTAVYYCANP |
| fused to SIRPa | YYDYVSYYAMDYWGOGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSEEELQVIQPDKSVLVAAGETATLRCTATSLIPVGPIQ |
| | WFRGAGPGRELIYNQKEGHFPRVTTVSDLTKRNNMDFSIRIGNITPADAGT |
| | YYCVKFRKGSPDDVEFKSGAGTELSVRAKPS |
| SEQ ID NO: 514 | QVQLVQSGSELKKPGASVKVSCKASGYTFTDYSMHWVRQAPGQGLKWM |
| anti-OX40 HC | GWINTETGEPTYADDFKGRFVFSLDTSVSTAYLQISSLKAEDTAVYYCANP |
| fused to TGFbR | YYDYVSYYAMDYWGOGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSTIPPHVQKSVNNDMIVTDNNGAVKFPQLCKFCDVR |
| | FSTCDNQKSCMSNCSITSICEKPQEVCVAVWRKNDENITLETVCHDPKLPY |
| | HDFILEDAASPKCIMKEKKKPGETFFMCSCSSDECNDNIIFSEEYNTSNPD |
| SEQ ID NO: 515 | QVQLVQSGSELKKPGASVKVSCKASGYTFTDYSMHWVRQAPGQGLKWM |
| anti-OX40 HC | GWINTETGEPTYADDFKGRFVFSLDTSVSTAYLQISSLKAEDTAVYYCANP |
| fused to TIM3 | YYDYVSYYAMDYWGOGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSSEVEYRAEVGQNAYLPCFYTPAAPGNLVPVCWGK |
| | GACPVFECGNVVLRTDERDVNYWTSRYWLNGDFRKGDVSLTIENVTLADS |
| | GIYCCRIQIPGIMNDEKFNLKLVIKPAKVTPAPTRQRDFTAAFPRMLTTRGH |
| | GPAETQTLGSLPDINLTQISTLANELRDSRLANDLRDSGATIRIG |
| SEQ ID NO: 516 | QVQLVQSGSELKKPGASVKVSCKASGYTFTDYSMHWVRQAPGQGLKWM |
| anti-OX40 HC | GWINTETGEPTYADDFKGRFVFSLDTSVSTAYLQISSLKAEDTAVYYCANP |
| fused to VEGFR | YYDYVSYYAMDYWGOGTTVTVSSASTKGPSVFPLAPSSKSTSGGTAALGC |
| ECD | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSSDTGRPFVEMYSEIPEIIHMTEGRELVIPCRVTSPNIT |
| | VTLKKFPLDTLIPDGKRIIWDSRKGFIISNATYKEIGLLTCEATVNGHLYKTN |
| | YLTHRQTNTIIDVVLSPSHGIELSVGEKLVLNCTARTELNVGIDFNWEYPSS |
| | KHQHKKLVNRDLKTQSGSEMKKFLSTLTIDGVTRSDQGLYTCAASSGLMT |
| | KKNSTFVRVHEK |
| SEQ ID NO: 517 | QVQLVQSGAEVKKPGSSVKVSCKASGYTFTDYAMHWVRQAPGQGLEWM |
| anti-ICOS HC | GLISIYSDHTNYNQKFQGRVTITADKSTSTAYMELSSLRSEDTAVYYCGRN |
| fused to BTLA | NYGNYGWYFDVWGQGTTVTVSSGGGGSGGGGSGGGGSKESCDVQLYIKR |
| ECD | QSEHSILAGDPFELECPVKYCANRPHVTWCKLNGTTCVKLEDRQTSWKEE |
| | KNISFFILHFEPVLPNDNGSYRCSANFQSNLIESHSTTLYVTDVKSASERPSK |
| | DEMAS |
| SEQ ID NO: 518 | EIVLTQSPATLSLSPGERATLSCSASSSVSYMHWYQQKPGQAPRLLIYDTSK |
| anti-ICOS LC | LASGIPARFSGSGSGTDYTLTISSLEPEDFAVYYCFQGSGYPYTFGOGTKLEI |
| fused to PD1 ECD | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKS |
| | FNRGECGGGGSGGGGSGGGGSPGWFLDSPDRPWNPPTFSPALLVVTEGDN |
| | ATFTCSFSNTSESFVLNWYRMSPSNQTDKLAAFPEDRSQPGQDCRFRVTQL |
| | PNGRDFHMSVVRARRNDSGTYLCGAISLAPKAQIKESLRAELRVTERRAEV |
| | PTAHPSPSPRPAGQFQTLV |
| SEQ ID NO: 519 | EIVLTQSPATLSLSPGERATLSCSASSSVSYMHWYQQKPGQAPRLLIYDTSK |
| anti-ICOS LC | LASGIPARFSGSGSGTDYTLTISSLEPEDFAVYYCFQGSGYPYTFGOGTKLEI |
| fused to | KRTVAAPSVFIFPPSDEQLKSGTASVVCLLNNFYPREAKVQWKVDNALQSG |
| SIGLEC10 ECD | NSQESVTEQDSKDSTYSLSSTLTLSKADYEKHKVYACEVTHQGLSSPVTKS |
| | FNRGECGGGGSGGGGSGGGGSMDGRFWIRVQESVMVPEGLCISVPCSFSY |
| | PRQDWTGSTPAYGYWFKAVTETTKGAPVATNHQSREVEMSTRGRFQLTG |
| | DPAKGNCSLVIRDAQMQDESQYFFRVERGSYVRYNFMNDGFFLKVTALTQ |
| | KPDVYIPETLEPGQPVTVICVFNWAFEECPPPSFSWTGAALSSQGTKPTTSH |
| | FSVLSFTPRPQDHNTDLTCHVDFSRKGVSAQRTVRLRVAYAPRDLVISISRD |
| | NTPALEPQPQGNVPYLEAQKGQFLRLLCAADSQPPATLSWVLQNRVLSSSH |
| | PWGPRPLGLELPGVKAGDSGRYTCRAENRLGSQQRALDLSVQYPPENLRV |
| | MVSQANRTVLENLGNGTSLPVLEGQSLCLVCVTHSSPPARLSWTQRGQVL |
| | SPSQPSDPGVLELPRVQVEHEGEFTCHARHPLGSQHVSLSLSVHYSPKLLGP |
| | SCSWEAEGLHCSCSSQASPAPSLRWWLGEELLEGNSSQDSFEVTPSSAGPW |
| | ANSSLSLHGGLSSGLRLRCEAWNVHGAQSGSILQLPDKKGLIST |
| SEQ ID NO: 520 | EIVLTQSPATLSLSPGERATLSCSASSSVSYMHWYQQKPGQAPRLLIYDTSK |
| anti-ICOS LC | LASGIPARFSGSGSGTDYTLTISSLEPEDFAVYYCFQGSGYPYTFGOGTKLEI |
| fused to SIRPa | KRTVAAPSVFIFPPSDEQLKSGTASVVCLLNNFYPREAKVQWKVDNALQSG |
| EC | NSQESVTEQDSKDSTYSLSSTLTLSKADYEKHKVYACEVTHQGLSSPVTKS |
| | FNRGECGGGGSGGGGSGGGGSEEELQVIQPDKSVLVAAGETATLRCTATSL |
| | IPVGPIQWFRGAGPGRELIYNQKEGHFPRVTTVSDLTKRNNMDFSIRIGNITP |
| | ADAGTYYCVKFRKGSPDDVEFKSGAGTELSVRAKPS |
| SEQ ID NO: 521 | EIVLTQSPATLSLSPGERATLSCSASSSVSYMHWYQQKPGQAPRLLIYDTSK |
| anti-ICOS LC | LASGIPARFSGSGSGTDYTLTISSLEPEDFAVYYCFQGSGYPYTFGOGTKLEI |
| fused to TIM3 | KRTVAAPSVFIFPPSDEQLKSGTASVVCLLNNFYPREAKVQWKVDNALQSG |
| ECD | NSQESVTEQDSKDSTYSLSSTLTLSKADYEKHKVYACEVTHQGLSSPVTKS |
| | FNRGECGGGGSGGGGSGGGGSSEVEYRAEVGQNAYLPCFYTPAAPGNLVP |
| | VCWGKGACPVFECGNVVLRTDERDVNYWTSRYWLNGDFRKGDVSLTIEN |
| | VTLADSGIYCCRIQIPG1MNDEKFNLKLVIKPAKVTPAPTRQRDFTAAFPRM |
| | LTTRGHGPAETQTLGSLPDINLTQISTLANELRDSRLANDLRDSGATIRIG |
| SEQ ID NO: 522 | QVQLVQSGAEVKKPGSSVKVSCKASGYTFTDYAMHWVRQAPGQGLEWM |
| anti-ICOS HC | GLISIYSDHTNYNQKFQGRVTITADKSTSTAYMELSSLRSEDTAVYYCGRN |
| fused to PD1 ECD | NYGNYGWYFDVWGQGTTVTVSSGGGGSGGGGSGGGGSPGWFLDSPDRP |
| | WNPPTFSPALLVVTEGDNATFTCSFSNTSESFVLNWYRMSPSNQTDKLAAF |
| | PEDRSQPGQDCRFRVTQLPNGRDFHMSVVRARRNDSGTYLCGAISLAPKA |
| | QIKESLRAELRVTERRAEVPTAHPSPSPRPAGQFQTLV |
| SEQ ID NO: 523 | EIVLTQSPATLSLSPGERATLSCSASSSVSYMHWYQQKPGQAPRLLIYDTSK |
| anti-ICOS LC | LASGIPARFSGSGSGTDYTLTISSLEPEDFAVYYCFQGSGYPYTFGOGTKLEI |
| fused to BTLA | KRTVAAPSVFIFPPSDEQLKSGTASVVCLLNNFYPREAKVQWKVDNALQSG |
| ECD | NSQESVTEQDSKDSTYSLSSTLTLSKADYEKHKVYACEVTHQGLSSPVTKS |
| | FNRGECGGGGSGGGGSGGGGSKESCDVQLYIKRQSEHSILAGDPFELECPV |
| | KYCANRPHVTWCKLNGTTCVKLEDRQTSWKEEKNISFFILHFEPVLPNDNG |
| | SYRCSANFQSNLIESHSTTLYVTDVKSASERPSKDEMAS |
| SEQ ID NO: 524 | QVQLVQSGAEVKKPGSSVKVSCKASGYTFTDYAMHWVRQAPGQGLEWM |
| anti-ICOS HC | GLISIYSDHTNYNQKFQGRVTITADKSTSTAYMELSSLRSEDTAVYYCGRN |
| fused to | NYGNYGWYFDVWGQGTTVTVSSGGGGSGGGGSGGGGSMDGRFWIRVQE |
| SIGLEC10 ECD | SVMVPEGLCISVPCSFSYPRQDWTGSTPAYGYWFKAVTETTKGAPVATNH |
| | QSREVEMSTRGRFQLTGDPAKGNCSLVIRDAQMQDESQYFFRVERGSYVR |
| | YNFMNDGFFLKVTALTQKPDVYIPETLEPGQPVTVICVFNWAFEECPPPSFS |
| | WTGAALSSQGTKPTTSHFSVLSFTPRPQDHNTDLTCHVDFSRKGVSAQRTV |
| | RLRVAYAPRDLVISISRDNTPALEPQPQGNVPYLEAQKGQFLRLLCAADSQ |
| | PPATLSWVLQNRVLSSSHPWGPRPLGLELPGVKAGDSGRYTCRAENRLGS |
| | QQRALDLSVQYPPENLRVMVSQANRTVLENLGNGTSLPVLEGQSLCLVCV |
| | THSSPPARLSWTQRGQVLSPSQPSDPGVLELPRVQVEHEGEFTCHARHPLG |
| | SQHVSLSLSVHYSPKLLGPSCSWEAEGLHCSCSSQASPAPSLRWWLGEELL |
| | EGNSSQDSFEVTPSSAGPWANSSLSLHGGLSSGLRLRCEAWNVHGAQSGSI |
| | LQLPDKKGLIST |
| SEQ ID NO: 525 | QVQLVQSGAEVKKPGSSVKVSCKASGYTFTDYAMHWVRQAPGQGLEWM |
| anti-ICOS HC | GUSTYSDHTNYNQKFQGRVTITADKSTSTAYMELSSLRSEDTAVYYCGRN |
| fused to SIRPa | NYGNYGWYFDVWGQGTTVTVSSGGGGSGGGGSGGGGSEEELQVIQPDKS |
| ECD | VLVAAGETATLRCTATSLIPVGPIQWFRGAGPGRELIYNQKEGHFPRVTTVS |
| | DLTKRNNMDFSIRIGNITPADAGTYYCVKFRKGSPDDVEFKSGAGTELSVR |
| | AKPS |
| SEQ ID NO: 526 | QVQLVQSGAEVKKPGSSVKVSCKASGYTFTDYAMHWVRQAPGQGLEWM |
| anti-ICOS HC | GLISIYSDHTNYNQKFQGRVTITADKSTSTAYMELSSLRSEDTAVYYCGRN |
| fused to TGFbR | NYGNYGWYFDVWGQGTTVTVSSGGGGSGGGGSGGGGSTIPPHVQKSVNN |
| ECD | DMIVTDNNGAVKFPQLCKFCDVRFSTCDNQKSCMSNCSITSICEKPQEVCV |
| | AVWRKNDENITLETVCHDPKLPYHDFILEDAASPKCIMKEKKKPGETFFMC |
| | SCSSDECNDNIIFSEEYNTSNPD |
| SEQ ID NO: 527 | QVQLVQSGAEVKKPGSSVKVSCKASGYTFTDYAMHWVRQAPGQGLEWM |
| anti-ICOS HC | GLISIYSDHTNYNQKFQGRVTITADKSTSTAYMELSSLRSEDTAVYYCGRN |
| fused to TIM3 | NYGNYGWYFDVWGQGTTVTVSSGGGGSGGGGSGGGGSSEVEYRAEVGQ |
| ECD | NAYLPCFYTPAAPGNLVPVCWGKGACPVFECGNVVLRTDERDVNYWTSR |
| | YWLNGDFRKGDVSLTIENVTLADSGIYCCRIQIPGIMNDEKFNLKLVIKPAK |
| | VTPAPTRQRDFTAAFPRMLTTRGHGPAETQTLGSLPDINLTQISTLANELRD |
| | SRLANDLRDSGATIRIG |
| SEQ ID NO: 528 | QVQLVQSGAEVKKPGSSVKVSCKASGYTFTDYAMHWVRQAPGQGLEWM |
| anti-ICOS HC | GLISIYSDHTNYNQKFQGRVTITADKSTSTAYMELSSLRSEDTAVYYCGRN |
| fused to VEGFR | NYGNYGWYFDVWGQGTTVTVSSGGGGSGGGGSGGGGSSDTGRPFVEMY |
| ECD | SEIPEIIHMTEGRELVIPCRVTSPNITVTLKKFPLDTLIPDGKRIIWDSRKGFIIS |
| | NATYKEIGLLTCEATVNGHLYKTNYLTHRQTNTIIDVVLSPSHGIELSVGEK |
| | LVLNCTARTELNVGIDFNWEYPSSKHQHKKLVNRDLKTQSGSEMKKFLST |
| | LTIDGVTRSDQGLYTCAASSGLMTKKNSTFVRVHEK |
| SEQ ID NO: 529 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| PD1ecd | DVKSASERPSKDEMASASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSPGWFLDSPDRPWNPPTFSPALLVVTEGDNATFTCSFSNTSESFVLN |
| | WYRMSPSNQTDKLAAFPEDRSQPGQDCRFRVTQLPNGRDFHMSVVRARR |
| | NDSGTYLCGAISLAPKAQIKESLRAELRVTERRAEVPTAHPSPSPRPAGQFQ |
| | TLV |
| SEQ ID NO: 530 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| SIGLEC10ecd | DVKSASERPSKDEMASASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSMDGRFWIRVQESVMVPEGLCISVPCSFSYPRQDWTGSTPAYGYW |
| | FKAVTETTKGAPVATNHQSREVEMSTRGRFQLTGDPAKGNCSLVIRDAQM |
| | QDESQYFFRVERGSYVRYNFMNDGFFLKVTALTQKPDVYIPETLEPGQPVT |
| | VICVFNWAFEECPPPSFSWTGAALSSQGTKPTTSHFSVLSFTPRPQDHNTDL |
| | TCHVDFSRKGVSAQRTVRLRVAYAPRDLVISISRDNTPALEPQPQGNVPYL |
| | EAQKGQFLRLLCAADSQPPATLSWVLQNRVLSSSHPWGPRPLGLELPGVK |
| | AGDSGRYTCRAENRLGSQQRALDLSVQYPPENLRVMVSQANRTVLENLG |
| | NGTSLPVLEGQSLCLVCVTHSSPPARLSWTQRGQVLSPSQPSDPGVLELPRV |
| | QVEHEGEFTCHARHPLGSQHVSLSLSVHYSPKLLGPSCSWEAEGLHCSCSS |
| | QASPAPSLRWWLGEELLEGNSSQDSFEVTPSSAGPWANSSLSLHGGLSSGL |
| | RLRCEAWNVHGAQSGSILQLPDKKGLIST |
| SEQ ID NO: 531 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| SIRPaecd | DVKSASERPSKDEMASASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSEEELQVIQPDKSVLVAAGETATLRCTATSLIPVGPIQWFRGAGPGR |
| | ELIYNQKEGHFPRVTTVSDLTKRNNMDFSIRIGNITPADAGTYYCVKFRKGS |
| | PDDVEFKSGAGTELSVRAKPS |
| SEQ ID NO: 532 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| TGFbRecd | DVKSASERPSKDEMASASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSTIPPHVQKSVNNDMIVTDNNGAVKFPQLCKFCDVRFSTCDNQKS |
| | CMSNCSITSICEKPQEVCVAVWRKNDENITLETVCHDPKLPYHDFILEDAAS |
| | PKCIMKEKKKPGETFFMCSCSSDECNDNIIFSEEYNTSNPD |
| SEQ ID NO: 533 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| TIM3ecd | DVKSASERPSKDEMASASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSEVEYRAEVGQNAYLPCFYTPAAPGNLVPVCWGKGACPVFECGN |
| | VVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGS |
| | LPDINLTQISTLANELRDSRLANDLRDSGATIRIG |
| SEQ ID NO: 534 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| VEGFRecd | DVKSASERPSKDEMASASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSDTGRPFVEMYSEIPEIIHMTEGRELVIPCRVTSPNITVTLKKFPLDT |
| | LIPDGKRIIWDSRKGFIISNATYKEIGLLTCEATVNGHLYKTNYLTHRQTNTI |
| | IDVVLSPSHGIELSVGEKLVLNCTARTELNVGIDFNWEYPSSKHQHKKLVN |
| | RDLKTQSGSEMKKFLSTLTIDGVTRSDQGLYTCAASSGLMTKKNSTFVRVH |
| | EK |
| SEQ ID NO: 535 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| BTLAecd | LCGAISLAPKAQIKESLRAELRVTERRAEVPTAHPSPSPRPAGQFQTLVAST |
| | KGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSC |
| | SVMHEALHNHYTQKSLSLSPGKGGGGSGGGGSGGGGSKESCDVQLYIKRQ |
| | SEHSILAGDPFELECPVKYCANRPHVTWCKLNGTTCVKLEDRQTSWKEEK |
| | NISFFILHFEPVLPNDNGSYRCSANFQSNLIESHSTTLYVTDVKSASERPSKD |
| | EMAS |
| SEQ ID NO: 536 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| SIGLEC10ecd | LCGAISLAPKAQIKESLRAELRVTERRAEVPTAHPSPSPRPAGQFQTLVAST |
| | KGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSC |
| | SVMHEALHNHYTQKSLSLSPGKGGGGSGGGGSGGGGSMDGRFWIRVQES |
| | VMVPEGLCISVPCSFSYPRQDWTGSTPAYGYWFKAVTETTKGAPVATNHQ |
| | SREVEMSTRGRFQLTGDPAKGNCSLVIRDAQMQDESQYFFRVERGSYVRY |
| | NFMNDGFFLKVTALTQKPDVYIPETLEPGQPVTVICVFNWAFEECPPPSFSW |
| | TGAALSSQGTKPTTSHFSVLSFTPRPQDHNTDLTCHVDFSRKGVSAQRTVR |
| | LRVAYAPRDLVISISRDNTPALEPQPQGNVPYLEAQKGQFLRLLCAADSQPP |
| | ATLSWVLQNRVLSSSHPWGPRPLGLELPGVKAGDSGRYTCRAENRLGSQQ |
| | RALDLSVQYPPENLRVMVSQANRTVLENLGNGTSLPVLEGQSLCLVCVTH |
| | SSPPARLSWTQRGQVLSPSQPSDPGVLELPRVQVEHEGEFTCHARHPLGSQ |
| | HVSLSLSVHYSPKLLGPSCSWEAEGLHCSCSSQASPAPSLRWWLGEELLEG |
| | NSSQDSFEVTPSSAGPWANSSLSLHGGLSSGLRLRCEAWNVHGAQSGSILQ |
| | LPDKKGLIST |
| SEQ ID NO: 537 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| SIRPaecd | LCGAISLAPKAQIKESLRAELRVTERRAEVPTAHPSPSPRPAGQFQTLVAST |
| | KGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSC |
| | SVMHEALHNHYTQKSLSLSPGKGGGGSGGGGSGGGGSEEELQVIQPDKSV |
| | LVAAGETATLRCTATSLIPVGPIQWFRGAGPGRELIYNQKEGHFPRVTTVSD |
| | LTKRNNMDFSIRIGNITPADAGTYYCVKFRKGSPDDVEFKSGAGTELSVRA |
| | KPS |
| SEQ ID NO: 538 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| TGFbRecd | LCGAISLAPKAQIKESLRAELRVTERRAEVPTAHPSPSPRPAGQFQTLVAST |
| | KGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSC |
| | SVMHEALHNHYTQKSLSLSPGKGGGGSGGGGSGGGGSTIPPHVQKSVNND |
| | MIVTDNNGAVKFPQLCKFCDVRFSTCDNQKSCMSNCSITSICEKPQEVCVA |
| | VWRKNDENITLETVCHDPKLPYHDFILEDAASPKCIMKEKKKPGETFFMCS |
| | CSSDECNDNIIFSEEYNTSNPD |
| SEQ ID NO: 539 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| TIM3ecd | LCGAISLAPKAQIKESLRAELRVTERRAEVPTAHPSPSPRPAGQFQTLVAST |
| | KGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSC |
| | SVMHEALHNHYTQKSLSLSPGKGGGGSGGGGSGGGGSSEVEYRAEVGQN |
| | AYLPCFYTPAAPGNLVPVCWGKGACPVFECGNVVLRTDERDVNYWTSRY |
| | WLNGDFRKGDVSLTIENVTLADSGIYCCRIQIPGIMNDEKFNLKLVIKPAKV |
| | TPAPTRQRDFTAAFPRMLTTRGHGPAETQTLGSLPDINLTQISTLANELRDS |
| | RLANDLRDSGATIRIG |
| SEQ ID NO: 540 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| VEGFRecd | LCGAISLAPKAQIKESLRAELRVTERRAEVPTAHPSPSPRPAGQFQTLVAST |
| | KGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSC |
| | SVMHEALHNHYTQKSLSLSPGKGGGGSGGGGSGGGGSSDTGRPFVEMYSE |
| | IPEIIHMTEGRELVIPCRVTSPNITVTLKKFPLDTLIPDGKRIIWDSRKGFIISN |
| | ATYKEIGLLTCEATVNGHLYKTNYLTHRQTNTIIDVVLSPSHGIELSVGEKL |
| | VLNCTARTELNVGIDFNWEYPSSKHQHKKLVNRDLKTQSGSEMKKFLSTL |
| | TIDGVTRSDQGLYTCAASSGLMTKKNSTFVRVHEK |
| SEQ ID NO: 541 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-BTLAecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSKESCDVQLYIKRQSEHSILAGDPFELECPVKYCANRPHVT |
| | WCKLNGTTCVKLEDRQTSWKEEKNISFFILHFEPVLPNDNGSYRCSANFQS |
| | NLIESHSTTLYVTDVKSASERPSKDEMAS |
| SEQ ID NO: 542 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-PD1ecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSPGWFLDSPDRPWNPPTFSPALLVVTEGDNATFTCSFSNTS |
| | ESFVLNWYRMSPSNQTDKLAAFPEDRSQPGQDCRFRVTQLPNGRDFHMSV |
| | VRARRNDSGTYLCGAISLAPKAQIKESLRAELRVTERRAEVPTAHPSPSPRP |
| | AGQFQTLV |
| SEQ ID NO: 543 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-SIRPaecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSEEELQVIQPDKSVLVAAGETATLRCTATSLIPVGPIQWFRG |
| | AGPGRELIYNQKEGHFPRVTTVSDLTKRNNMDFSIRIGNITPADAGTYYCV |
| | KFRKGSPDDVEFKSGAGTELSVRAKPS |
| SEQ ID NO: 544 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-TGFbRecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSTIPPHVQKSVNNDMIVTDNNGAVKFPQLCKFCDVRFSTC |
| | DNQKSCMSNCSITSICEKPQEVCVAVWRKNDENITLETVCHDPKLPYHDFIL |
| | EDAASPKCIMKEKKKPGETFFMCSCSSDECNDNIIFSEEYNTSNPD |
| SEQ ID NO: 545 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-TIM3ecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSSEVEYRAEVGQNAYLPCFYTPAAPGNLVPVCWGKGACP |
| | VFECGNVVLRTDERDVNYWTSRYWLNGDFRKGDVSLTIENVTLADSGIYC |
| | CRIQIPGIMNDEKFNLKLVIKPAKVTPAPTRQRDFTAAFPRMLTTRGHGPAE |
| | TQTLGSLPDINLTQISTLANELRDSRLANDLRDSGATIRIG |
| SEQ ID NO: 546 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-VEGFRecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGKGGGG |
| | SGGGGSGGGGSSDTGRPFVEMYSEIPEIIHMTEGRELVIPCRVTSPNITVTLK |
| | KFPLDTLIPDGKRIIWDSRKGFIISNATYKEIGLLTCEATVNGHLYKTNYLTH |
| | RQTNTIIDVVLSPSHGIELSVGEKLVLNCTARTELNVGIDFNWEYPSSKHQH |
| | KKLVNRDLKTQSGSEMKKFLSTLTIDGVTRSDQGLYTCAASSGLMTKKNS |
| | TFVRVHEK |
| SEQ ID NO: 547 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| BTLAecd | FKSGAGTELSVRAKPSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSKESCDVQLYIKRQSEHSILAGDPFELECPVKYCANRPHVTWCKLN |
| | GTTCVKLEDRQTSWKEEKNISFFILHFEPVLPNDNGSYRCSANFQSNLIESHS |
| | TTLYVTDVKSASERPSKDEMAS |
| SEQ ID NO: 548 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| PD1ecd | FKSGAGTELSVRAKPSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSPGWFLDSPDRPWNPPTFSPALLVVTEGDNATFTCSFSNTSESFVLN |
| | WYRMSPSNQTDKLAAFPEDRSQPGQDCRFRVTQLPNGRDFHMSVVRARR |
| | NDSGTYLCGAISLAPKAQIKESLRAELRVTERRAEVPTAHPSPSPRPAGQFQ |
| | TLV |
| SEQ ID NO: 549 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| SIGLEC10ecd | FKSGAGTELSVRAKPSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSMDGRFWIRVQESVMVPEGLCISVPCSFSYPRQDWTGSTPAYGYW |
| | FKAVTETTKGAPVATNHQSREVEMSTRGRFQLTGDPAKGNCSLVIRDAQM |
| | QDESQYFFRVERGSYVRYNFMNDGFFLKVTALTQKPDVYIPETLEPGQPVT |
| | VICVFNWAFEECPPPSFSWTGAALSSQGTKPTTSHFSVLSFTPRPQDHNTDL |
| | TCHVDFSRKGVSAQRTVRLRVAYAPRDLVISISRDNTPALEPQPQGNVPYL |
| | EAQKGQFLRLLCAADSQPPATLSWVLQNRVLSSSHPWGPRPLGLELPGVK |
| | AGDSGRYTCRAENRLGSQQRALDLSVQYPPENLRVMVSQANRTVLENLG |
| | NGTSLPVLEGQSLCLVCVTHSSPPARLSWTQRGQVLSPSQPSDPGVLELPRV |
| | QVEHEGEFTCHARHPLGSQHVSLSLSVHYSPKLLGPSCSWEAEGLHCSCSS |
| | QASPAPSLRWWLGEELLEGNSSQDSFEVTPSSAGPWANSSLSLHGGLSSGL |
| | RLRCEAWNVHGAQSGSILQLPDKKGLIST |
| SEQ ID NO: 550 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| TGFbRecd | FKSGAGTELSVRAKPSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSTIPPHVQKSVNNDMIVTDNNGAVKFPQLCKFCDVRFSTCDNQKS |
| | CMSNCSITSICEKPQEVCVAVWRKNDENITLETVCHDPKLPYHDFILEDAAS |
| | PKCIMKEKKKPGETFFMCSCSSDECNDNIIFSEEYNTSNPD |
| SEQ ID NO: 551 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| TIM3ecd | FKSGAGTELSVRAKPSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSEVEYRAEVGQNAYLPCFYTPAAPGNLVPVCWGKGACPVFECGN |
| | VVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGS |
| | LPDINLTQISTLANELRDSRLANDLRDSGATIRIG |
| SEQ ID NO: 552 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| VEGFRec | FKSGAGTELSVRAKPSASTKGPSVFPLAPSSKSTSGGTAALGCLVKDYFPEP |
| | VTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGKGGGGSGGGG |
| | SGGGGSSDTGRPFVEMYSEIPEIIHMTEGRELVIPCRVTSPNITVTLKKFPLDT |
| | LIPDGKRIIWDSRKGFIISNATYKEIGLLTCEATVNGHLYKTNYLTHRQTNTI |
| | IDVVLSPSHGIELSVGEKLVLNCTARTELNVGIDFNWEYPSSKHQHKKLVN |
| | RDLKTQSGSEMKKFLSTLTIDGVTRSDQGLYTCAASSGLMTKKNSTFVRVH |
| | EK |
| SEQ ID NO: 553 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| BTLAecd | KKKPGETFFMCSCSSDECNDNIIFSEEYNTSNPDASTKGPSVFPLAPSSKSTS |
| | GGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKS |
| | LSLSPGKGGGGSGGGGSGGGGSKESCDVQLYIKRQSEHSILAGDPFELECPV |
| | KYCANRPHVTWCKLNGTTCVKLEDRQTSWKEEKNISFFILHFEPVLPNDNG |
| | SYRCSANFQSNLIESHSTTLYVTDVKSASERPSKDEMAS |
| SEQ ID NO: 554 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| PD1ecd | KKKPGETFFMCSCSSDECNDNIIFSEEYNTSNPDASTKGPSVFPLAPSSKSTS |
| | GGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKS |
| | LSLSPGKGGGGSGGGGSGGGGSPGWFLDSPDRPWNPPTFSPALLVVTEGD |
| | NATFTCSFSNTSESFVLNWYRMSPSNQTDKLAAFPEDRSQPGQDCRFRVTQ |
| | LPNGRDFHMSVVRARRNDSGTYLCGAISLAPKAQIKESLRAELRVTERRAE |
| | VPTAHPSPSPRPAGQFQTLV |
| SEQ ID NO: 555 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| SIGLEC10ecd | KKKPGETFFMCSCSSDECNDNIIFSEEYNTSNPDASTKGPSVFPLAPSSKSTS |
| | GGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKS |
| | LSLSPGKGGGGSGGGGSGGGGSMDGRFWIRVQESVMVPEGLCISVPCSFSY |
| | PRQDWTGSTPAYGYWFKAVTETTKGAPVATNHQSREVEMSTRGRFQLTG |
| | DPAKGNCSLVIRDAQMQDESQYFFRVERGSYVRYNFMNDGFFLKVTALTQ |
| | KPDVYIPETLEPGQPVTVICVFNWAFEECPPPSFSWTGAALSSQGTKPTTSH |
| | FSVLSFTPRPQDHNTDLTCHVDFSRKGVSAQRTVRLRVAYAPRDLVISISRD |
| | NTPALEPQPQGNVPYLEAQKGQFLRLLCAADSQPPATLSWVLQNRVLSSSH |
| | PWGPRPLGLELPGVKAGDSGRYTCRAENRLGSQQRALDLSVQYPPENLRV |
| | MVSQANRTVLENLGNGTSLPVLEGQSLCLVCVTHSSPPARLSWTQRGQVL |
| | SPSQPSDPGVLELPRVQVEHEGEFTCHARHPLGSQHVSLSLSVHYSPKLLGP |
| | SCSWEAEGLHCSCSSQASPAPSLRWWLGEELLEGNSSQDSFEVTPSSAGPW |
| | ANSSLSLHGGLSSGLRLRCEAWNVHGAQSGSILQLPDKKGLIST |
| SEQ ID NO: 556 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| SIRPaecd | KKKPGETFFMCSCSSDECNDNIIFSEEYNTSNPDASTKGPSVFPLAPSSKSTS |
| | GGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKS |
| | LSLSPGKGGGGSGGGGSGGGGSEEELQVIQPDKSVLVAAGETATLRCTATS |
| | LIPVGPIQWFRGAGPGRELIYNQKEGHFPRVTTVSDLTKRNNMDFSIRIGNIT |
| | PADAGTYYCVKFRKGSPDDVEFKSGAGTELSVRAKPS |
| SEQ ID NO: 557 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| TIM3ecd | KKKPGETFFMCSCSSDECNDNIIFSEEYNTSNPDASTKGPSVFPLAPSSKSTS |
| | GGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKS |
| | LSLSPGKGGGGSGGGGSGGGGSSEVEYRAEVGQNAYLPCFYTPAAPGNLV |
| | PVCWGKGACPVFECGNVVLRTDERDVNYWTSRYWLNGDFRKGDVSLTIE |
| | NVTLADSGIYCCRIQIPGIMNDEKFNLKLVIKPAKVTPAPTRQRDFTAAFPR |
| | MLTTRGHGPAETQTLGSLPDINLTQISTLANELRDSRLANDLRDSGATIRIG |
| SEQ ID NO: 558 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| VEGFRecd | KKKPGETFFMCSCSSDECNDNIIFSEEYNTSNPDASTKGPSVFPLAPSSKSTS |
| | GGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKS |
| | LSLSPGKGGGGSGGGGSGGGGSSDTGRPFVEMYSEIPEIIHMTEGRELVIPC |
| | RVTSPNITVTLKKFPLDTLIPDGKRIIWDSRKGFIISNATYKEIGLLTCEATVN |
| | GHLYKTNYLTHRQTNTIIDVVLSPSHGIELSVGEKLVLNCTARTELNVGIDF |
| | NWEYPSSKHQHKKLVNRDLKTQSGSEMKKFLSTLTIDGVTRSDQGLYTCA |
| | ASSGLMTKKNSTFVRVHEK |
| SEQ ID NO: 559 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| BTLAecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPSSKSTSGGTAA |
| | LGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSKESCDVQLYIKRQSEHSILAGDPFELECPVKYCAN |
| | RPHVTWCKLNGTTCVKLEDRQTSWKEEKNISFFILHFEPVLPNDNGSYRCS |
| | ANFQSNLIESHSTTLYVTDVKSASERPSKDEMAS |
| SEQ ID NO: 560 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| PD1ecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPSSKSTSGGTAA |
| | LGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSPGWFLDSPDRPWNPPTFSPALLVVTEGDNATFTC |
| | SFSNTSESFVLNWYRMSPSNQTDKLAAFPEDRSQPGQDCRFRVTQLPNGRD |
| | FHMSVVRARRNDSGTYLCGAISLAPKAQIKESLRAELRVTERRAEVPTAHP |
| | SPSPRPAGQFQTLV |
| SEQ ID NO: 561 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| SIGLEC10ecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPSSKSTSGGTAA |
| | LGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSMDGRFW1RVQESVMVPEGLCISVPCSFSYPRQDW |
| | TGSTPAYGYWFKAVTETTKGAPVATNHQSREVEMSTRGRFQLTGDPAKG |
| | NCSLVIRDAQMQDESQYFFRVERGSYVRYNFMNDGFFLKVTALTQKPDVY |
| | IPETLEPGQPVTVICVFNWAFEECPPPSFSWTGAALSSQGTKPTTSHFSVLSF |
| | TPRPQDHNTDLTCHVDFSRKGVSAQRTVRLRVAYAPRDLVISISRDNTPAL |
| | EPQPQGNVPYLEAQKGQFLRLLCAADSQPPATLSWVLQNRVLSSSHPWGP |
| | RPLGLELPGVKAGDSGRYTCRAENRLGSQQRALDLSVQYPPENLRVMVSQ |
| | ANRTVLENLGNGTSLPVLEGQSLCLVCVTHSSPPARLSWTQRGQVLSPSQP |
| | SDPGVLELPRVQVEHEGEFTCHARHPLGSQHVSLSLSVHYSPKLLGPSCSW |
| | EAEGLHCSCSSQASPAPSLRWWLGEELLEGNSSQDSFEVTPSSAGPWANSS |
| | LSLHGGLSSGLRLRCEAWNVHGAQSGSILQLPDKKGLIST |
| SEQ ID NO: 562 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| SIRPaecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPSSKSTSGGTAA |
| | LGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSEEELQVIQPDKSVLVAAGETATLRCTATSLIPVGPI |
| | QWFRGAGPGRELIYNQKEGHFPRVTTVSDLTKRNNMDFSIRIGNITPADAG |
| | TYYCVKFRKGSPDDVEFKSGAGTELSVRAKPS |
| SEQ ID NO: 563 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| TGFbRecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPSSKSTSGGTAA |
| | LGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSTIPPHVQKSVNNDMIVTDNNGAVKFPQLCKFCDV |
| | RFSTCDNQKSCMSNCSITSICEKPQEVCVAVWRKNDENITLETVCHDPKLP |
| | YHDFILEDAASPKCIMKEKKKPGETFFMCSCSSDECNDNIIFSEEYNTSNPD |
| SEQ ID NO: 564 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| VEGFRecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPSSKSTSGGTAA |
| | LGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | KGGGGSGGGGSGGGGSSDTGRPFVEMYSEIPEIIHMTEGRELVIPCRVTSPNI |
| | TVTLKKFPLDTLIPDGKRIIWDSRKGFIISNATYKEIGLLTCEATVNGHLYKT |
| | NYLTHRQTNTIIDVVLSPSHGIELSVGEKLVLNCTARTELNVGIDFNWEYPS |
| | SKHQHKKLVNRDLKTQSGSEMKKFLSTLTIDGVTRSDQGLYTCAASSGLM |
| | TKKNSTFVRVHEK |
| SEQ ID NO: 565 | SDTGRPFVEMYSEIPEIIHMTEGRELVIPCRVTSPNITVTLKKFPLDTLIPDGK |
| VEGFRecd-Fc - | RIIWDSRKGFIISNATYKEIGLLTCEATVNGHLYKTNYLTHRQTNTIIDVVLS |
| BTLAecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSKESCDVQLYIKRQS |
| | EHSILAGDPFELECPVKYCANRPHVTWCKLNGTTCVKLEDRQTSWKEEKNI |
| | SFFILHFEPVLPNDNGSYRCSANFQSNLIESHSTTLYVTDVKSASERPSKDEM |
| | AS |
| SEQ ID NO: 566 | SDTGRPFVEMYSEIPEIIHMTEGRELVIPCRVTSPNITVTLKKFPLDTLIPDGK |
| VEGFRecd-Fc - | RIIWDSRKGFIISNATYKEIGLLTCEATVNGHLYKTNYLTHRQTNTIIDVVLS |
| PD1ecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSPGWFLDSPDRPWN |
| | PPTFSPALLVVTEGDNATFTCSFSNTSESFVLNWYRMSPSNQTDKLAAFPED |
| | RSQPGQDCRFRVTQLPNGRDFHMSVVRARRNDSGTYLCGAISLAPKAQIKE |
| | SLRAELRVTERRAEVPTAHPSPSPRPAGQFQTLV |
| SEQ ID NO: 567 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| SIGLEC10ecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSMDGRFWIRVQESV |
| | MVPEGLCISVPCSFSYPRQDWTGSTPAYGYWFKAVTETTKGAPVATNHQS |
| | REVEMSTRGRFQLTGDPAKGNCSLVIRDAQMQDESQYFFRVERGSYVRYN |
| | FMNDGFFLKVTALTQKPDVYIPETLEPGQPVTVICVFNWAFEECPPPSFSWT |
| | GAALSSQGTKPTTSHFSVLSFTPRPQDHNTDLTCHVDFSRKGVSAQRTVRL |
| | RVAYAPRDLVISISRDNTPALEPQPQGNVPYLEAQKGQFLRLLCAADSQPP |
| | ATLSWVLQNRVLSSSHPWGPRPLGLELPGVKAGDSGRYTCRAENRLGSQQ |
| | RALDLSVQYPPENLRVMVSQANRTVLENLGNGTSLPVLEGQSLCLVCVTH |
| | SSPPARLSWTQRGQVLSPSQPSDPGVLELPRVQVEHEGEFTCHARHPLGSQ |
| | HVSLSLSVHYSPKLLGPSCSWEAEGLHCSCSSQASPAPSLRWWLGEELLEG |
| | NSSQDSFEVTPSSAGPWANSSLSLHGGLSSGLRLRCEAWNVHGAQSGSILQ |
| | LPDKKGLIST |
| SEQ ID NO: 568 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| SIRPaecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSEEELQVIQPDKSVL |
| | VAAGETATLRCTATSLIPVGPIQWFRGAGPGRELIYNQKEGHFPRVTTVSDL |
| | TKRNNMDFSIRIGNITPADAGTYYCVKFRKGSPDDVEFKSGAGTELSVRAK |
| | PS |
| SEQ ID NO: 569 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| TGFbRecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSTIPPHVQKSVNND |
| | MIVTDNNGAVKFPQLCKFCDVRFSTCDNQKSCMSNCSITSICEKPQEVCVA |
| | VWRKNDENITLETVCHDPKLPYHDFILEDAASPKCIMKEKKKPGETFFMCS |
| | CSSDECNDNIIFSEEYNTSNPD |
| SEQ ID NO: 570 | SDTGRPFVEMYSEIPEIIHMTEGRELVIPCRVTSPNITVTLKKFPLDTLIPDGK |
| VEGFRecd-Fc - | RIIWDSRKGFIISNATYKEIGLLTCEATVNGHLYKTNYLTHRQTNTIIDVVLS |
| TIM3ecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSSEVEYRAEVGQNA |
| | YLPCFYTPAAPGNLVPVCWGKGACPVFECGNVVLRTDERDVNYWTSRYW |
| | LNGDFRKGDVSLTIENVTLADSGIYCCRIQIPGIMNDEKFNLKLVIKPAKVTP |
| | APTRQRDFTAAFPRMLTTRGHGPAETQTLGSLPDINLTQISTLANELRDSRL |
| | ANDLRDSGATIRIG |
| SEQ ID NO: 571 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| IL12ecd | DVKSASERPSKDEMASASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPSCPAPEFLGGPSVFLFPPKPKDTLMISRTPEVT |
| | CVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGKGGGGSGGGGSGGG |
| | GSIWELKKDVYVVELDWYPDAPGEMVVLTCDTPEEDGITWTLDQSSEVLG |
| | SGKTLTIQVKEFGDAGQYTCHKGGEVLSHSLLLLHKKEDGIWSTDILKDQK |
| | EPKNKTFLRCEAKNYSGRFTCWWLTTISTDLTFSVKSSRGSSDPQGVTCGA |
| | ATLSAERVRGDNKEYEYSVECQEDSACPAAEESLPIEVMVDAVHKLKYEN |
| | YTSSFFIRDIIKPDPPKNLQLKPLKNSRQVEVSWEYPDTWSTPHSYFSLTFCV |
| | QVQGKSKREKKDRVFTDKTSATVICRKNASISVRAQDRYYSSSWSEWASV |
| | PCSGGGGSGGGGSGGGGSRNLPVATPDPGMFPCLHHSQNLLRAVSNMLQK |
| | ARQTLEFYPCTSEEIDHEDITKDKTSTVEACLPLELTKNESCLNSRETSFITN |
| | GSCLASRKTSFMNIALCLSSIYEDLKMYQVEFKTMNAKLLMDPKRQIFLDQ |
| | NMLAVIDELMQALNFNSETVPQKSSLEEPDFYKTKIKLCILLHAFRIRAVTID |
| | RVMSYLNAS |
| SEQ ID NO: 572 | IWELKKDVYVVELDWYPDAPGEMVVLTCDTPEEDGITWTLDQSSEVLGSG |
| IL12ecd-Fc- | KTLTIQVKEFGDAGQYTCHKGGEVLSHSLLLLHKKEDGIWSTDILKDQKEP |
| BTLAecd | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMNIALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNASASTKGPSVFPLAPCSRSTSESTAALGCLVKDYFPEPVTVSWNSG |
| | ALTSGVHTFPAVLQSSGLYSLSSVVTVPSSSLGTKTYTCNVDHKPSNTKVD |
| | KRVESKYGPPCPSCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQE |
| | GNVFSCSVMHEALHNHYTQKSLSLSLGKGGGGSGGGGSGGGGSKESCDV |
| | QLYIKRQSEHSILAGDPFELECPVKYCANRPHVTWCKLNGTTCVKLEDRQT |
| | SWKEEKNISFFILHEEPVLPNDNGSYRCSANFQSNLIESHSTTLYVTDVKSAS |
| | ERPSKDEMAS |
| SEQ ID NO: 573 | KESCDVQLYIKRQSEHSILAGDPFELECPVKYCANRPHVTWCKLNGTTCVK |
| BTLAecd-Fc- | LEDRQTSWKEEKNISFFILHFEPVLPNDNGSYRCSANFQSNLIESHSTTLYVT |
| IL15ecd | DVKSASERPSKDEMASASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPSCPAPEFLGGPSVFLFPPKPKDTLMISRTPEVT |
| | CVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGKGGGGSGGGGSGGG |
| | GSNWVNVISDLKKIEDLIQSMHIDATLYTESDVHPSCKVTAMKCFLLELQVI |
| | SLESGDASIHDTVENLIILANNSLSSNGNVTESGCKECEELEEKNIKEFLQSF |
| | VHIVQMFINTS |
| SEQ ID NO: 574 | NWVNVISDLKKIEDLIQSMHIDATLYTESDVHPSCKVTAMKCFLLELQVISL |
| IL15ecd-Fc- | ESGDASIHDTVENLIILANNSLSSNGNVTESGCKECEELEEKNIKEFLQSFVH |
| BTLAecd | IVQMFINTSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKV |
| | DKRVESKYGPPCPSCPAPEFLGGPSVFLFPPKPKDTLMISRTPEVTCVVVDV |
| | SQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQ |
| | EGNVFSCSVMHEALHNHYTQKSLSLSLGKGGGGSGGGGSGGGGSKESCDV |
| | QLYIKRQSEHSILAGDPFELECPVKYCANRPHVTWCKLNGTTCVKLEDRQT |
| | SWKEEKNISFFILHEEPVLPNDNGSYRCSANFQSNLIESHSTTLYVTDVKSAS |
| | ERPSKDEMAS |
| SEQ ID NO: 575 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| IL12ecd | LCGAISLAPKAQIKESLRAELRVTERRAEVPTAHPSPSPRPAGQFQTLVAST |
| | KGPSVFPLAPCSRSTSESTAALGCLVKDYFPEPVTVSWNSGALTSGVHTFPA |
| | VLQSSGLYSLSSVVTVPSSSLGTKTYTCNVDHKPSNTKVDKRVESKYGPPC |
| | PSCPAPEFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCKVSNKG |
| | LPSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSIWELKKDVYVVELDWYP |
| | DAPGEMVVLTCDTPEEDGITWTLDQSSEVLGSGKTLTIQVKEFGDAGQYTC |
| | HKGGEVLSHSLLLLHKKEDGIWSTDILKDQKEPKNKTFLRCEAKNYSGRFT |
| | CWWLTTISTDLTFSVKSSRGSSDPQGVTCGAATLSAERVRGDNKEYEYSVE |
| | CQEDSACPAAEESLPIEVMVDAVHKLKYENYTSSFFIRDIIKPDPPKNLQLKP |
| | LKNSRQVEVSWEYPDTWSTPHSYFSLTFCVQVQGKSKREKKDRVFTDKTS |
| | ATVICRKNASISVRAQDRYYSSSWSEWASVPCSGGGGSGGGGSGGGGSRN |
| | LPVATPDPGMFPCLHHSQNLLRAVSNMLQKARQTLEFYPCTSEEIDHEDIT |
| | KDKTSTVEACLPLELTKNESCLNSRETSFITNGSCLASRKTSFMMALCLSSIY |
| | EDLKMYQVEFKTMNAKLLMDPKRQIFLDQNMLAVIDELMQALNFNSETV |
| | PQKSSLEEPDFYKTKIKLCILLHAFRIRAVTIDRVMSYLNAS |
| SEQ ID NO: 576 | IWELKKDVYVVELDWYPDAPGEMVVLTCDTPEEDGITWTLDQSSEVLGSG |
| IL12ecd-Fc- | KTLTIQVKEFGDAGQYTCHKGGEVLSHSLLLLHKKEDGIWSTDILKDQKEP |
| PD1ecd | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMMALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNASASTKGPSVFPLAPCSRSTSESTAALGCLVKDYFPEPVTVSWNSG |
| | ALTSGVHTFPAVLQSSGLYSLSSVVTVPSSSLGTKTYTCNVDHKPSNTKVD |
| | KRVESKYGPPCPSCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQE |
| | GNVFSCSVMHEALHNHYTQKSLSLSLGKGGGGSGGGGSGGGGSPGWFLD |
| | SPDRPWNPPTFSPALLVVTEGDNATFTCSFSNTSESFVLNWYRMSPSNQTD |
| | KLAAFPEDRSQPGQDCRFRVTQLPNGRDFHMSVVRARRNDSGTYLCGAIS |
| | LAPKAQIKESLRAELRVTERRAEVPTAHPSPSPRPAGQFQTLV |
| SEQ ID NO: 577 | PGWFLDSPDRPWNPPTFSPALLVVTEGDNATFTCSFSNTSESFVLNWYRMS |
| PD1ecd-Fc- | PSNQTDKLAAFPEDRSQPGQDCRFRVTQLPNGRDFHMSVVRARRNDSGTY |
| IL15ecd | LCGAISLAPKAQIKESLRAELRVTERRAEVPTAHPSPSPRPAGQFQTLVAST |
| | KGPSVFPLAPCSRSTSESTAALGCLVKDYFPEPVTVSWNSGALTSGVHTFPA |
| | VLQSSGLYSLSSVVTVPSSSLGTKTYTCNVDHKPSNTKVDKRVESKYGPPC |
| | PSCPAPEFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCKVSNKG |
| | LPSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSNWVNVISDLKKIEDLIQS |
| | MHIDATLYTESDVHPSCKVTAMKCFLLELQVISLESGDASIHDTVENLIILA |
| | NNSLSSNGNVTESGCKECEELEEKNIKEFLQSFVHIVQMFINTS |
| SEQ ID NO: 578 | NWVNVISDLKKIEDLIQSMHIDATLYTESDVHPSCKVTAMKCFLLELQVISL |
| IL15ecd-Fc- | ESGDASIHDTVENLIILANNSLSSNGNVTESGCKECEELEEKNIKEFLQSFVH |
| PD1ecd | IVQMFINTSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKV |
| | DKRVESKYGPPCPSCPAPEFLGGPSVFLFPPKPKDTLMISRTPEVTCVVVDV |
| | SQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQ |
| | EGNVFSCSVMHEALHNHYTQKSLSLSLGKGGGGSGGGGSGGGGSPGWFL |
| | DSPDRPWNPPTFSPALLVVTEGDNATFTCSFSNTSESFVLNWYRMSPSNQT |
| | DKLAAFPEDRSQPGQDCRFRVTQLPNGRDFHMSVVRARRNDSGTYLCGAI |
| | SLAPKAQIKESLRAELRVTERRAEVPTAHPSPSPRPAGQFQTLV |
| SEQ ID NO: 579 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-IL12ecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSIWELKKDVYVVELDWYPDAPGEMVVLTCDTPEEDGITWTLD |
| | QSSEVLGSGKTLTIQVKEFGDAGQYTCHKGGEVLSHSLLLLHKKEDGIWST |
| | DILKDQKEPKNKTFLRCEAKNYSGRFTCWWLTTISTDLTFSVKSSRGSSDPQ |
| | GVTCGAATLSAERVRGDNKEYEYSVECQEDSACPAAEESLPIEVMVDAVH |
| | KLKYENYTSSFFIRDIIKPDPPKNLQLKPLKNSRQVEVSWEYPDTWSTPHSY |
| | FSLTFCVQVQGKSKREKKDRVFTDKTSATVICRKNASISVRAQDRYYSSSW |
| | SEWASVPCSGGGGSGGGGSGGGGSRNLPVATPDPGMFPCLHHSQNLLRAV |
| | SNMLQKARQTLEFYPCTSEEIDHEDITKDKTSTVEACLPLELTKNESCLNSR |
| | ETSFITNGSCLASRKTSFMMALCLSSIYEDLKMYQVEFKTMNAKLLMDPKR |
| | QIFLDQNMLAVIDELMQALNFNSETVPQKSSLEEPDFYKTKIKLCILLHAFRI |
| | RAVTIDRVMSYLNAS |
| SEQ ID NO: 580 | IWELKKDVYVVELDWYPDAPGEMVVLTCDTPEEDGITWTLDQSSEVLGSG |
| IL12ecd-Fc- | KTLTIQVKEFGDAGQYTCHKGGEVLSHSLLLLHKKEDGIWSTDILKDQKEP |
| SIGLEC10ecd | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMMALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNASASTKGPSVFPLAPCSRSTSESTAALGCLVKDYFPEPVTVSWNSG |
| | ALTSGVHTFPAVLQSSGLYSLSSVVTVPSSSLGTKTYTCNVDHKPSNTKVD |
| | KRVESKYGPPCPSCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQE |
| | GNVFSCSVMHEALHNHYTQKSLSLSLGKGGGGSGGGGSGGGGSMDGRFW |
| | IRVQESVMVPEGLCISVPCSFSYPRQDWTGSTPAYGYWFKAVTETTKGAPV |
| | ATNHQSREVEMSTRGRFQLTGDPAKGNCSLVIRDAQMQDESQYFFRVERG |
| | SYVRYNFMNDGFFLKVTALTQKPDVYIPETLEPGQPVTVICVFNWAFEECP |
| | PPSFSWTGAALSSQGTKPTTSHFSVLSFTPRPQDHNTDLTCHVDFSRKGVSA |
| | QRTVRLRVAYAPRDLVISISRDNTPALEPQPQGNVPYLEAQKGQFLRLLCA |
| | ADSQPPATLSWVLQNRVLSSSHPWGPRPLGLELPGVKAGDSGRYTCRAEN |
| | RLGSQQRALDLSVQYPPENLRVMVSQANRTVLENLGNGTSLPVLEGQSLC |
| | LVCVTHSSPPARLSWTQRGQVLSPSQPSDPGVLELPRVQVEHEGEFTCHAR |
| | HPLGSQHVSLSLSVHYSPKLLGPSCSWEAEGLHCSCSSQASPAPSLRWWLG |
| | EELLEGNSSQDSFEVTPSSAGPWANSSLSLHGGLSSGLRLRCEAWNVHGAQ |
| | SGSILQLPDKKGLIST |
| SEQ ID NO: 581 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-IL15ecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSNWVNVISDLKKIEDLIQSMHIDATLYTESDVHPSCKVTAMKCF |
| | LLELQVISLESGDASIHDTVENLIILANNSLSSNGNVTESGCKECEELEEKNI |
| | KEFLQSFVHIVQMFINTS |
| SEQ ID NO: 582 | NWVNVISDLKKIEDLIQSMHIDATLYTESDVHPSCKVTAMKCFLLELQVISL |
| IL15ecd-Fc- | ESGDASIHDTVENLIILANNSLSSNGNVTESGCKECEELEEKNIKEFLQSFVH |
| SIGLEC10ecd | IVQMFINTSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKV |
| | DKRVESKYGPPCPSCPAPEFLGGPSVFLFPPKPKDTLMISRTPEVTCVVVDV |
| | SQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQ |
| | EGNVFSCSVMHEALHNHYTQKSLSLSLGKGGGGSGGGGSGGGGSMDGRF |
| | WIRVQESVMVPEGLCISVPCSFSYPRQDWTGSTPAYGYWFKAVTETTKGA |
| | PVATNHQSREVEMSTRGRFQLTGDPAKGNCSLVIRDAQMQDESQYFFRVE |
| | RGSYVRYNFMNDGFFLKVTALTQKPDVYIPETLEPGQPVTVICVFNWAFEE |
| | CPPPSFSWTGAALSSQGTKPTTSHFSVLSFTPRPQDHNTDLTCHVDFSRKGV |
| | SAQRTVRLRVAYAPRDLVISISRDNTPALEPQPQGNVPYLEAQKGQFLRLL |
| | CAADSQPPATLSWVLQNRVLSSSHPWGPRPLGLELPGVKAGDSGRYTCRA |
| | ENRLGSQQRALDLSVQYPPENLRVMVSQANRTVLENLGNGTSLPVLEGQS |
| | LCLVCVTHSSPPARLSWTQRGQVLSPSQPSDPGVLELPRVQVEHEGEFTCH |
| | ARHPLGSQHVSLSLSVHYSPKLLGPSCSWEAEGLHCSCSSQASPAPSLRWW |
| | LGEELLEGNSSQDSFEVTPSSAGPWANSSLSLHGGLSSGLRLRCEAWNVHG |
| | AQSGSILQLPDKKGLIST |
| SEQ ID NO: 583 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| IL12ecd | FKSGAGTELSVRAKPSASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPSCPAPEFLGGPSVFLFPPKPKDTLMISRTPEVT |
| | CVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGKGGGGSGGGGSGGG |
| | GSIWELKKDVYVVELDWYPDAPGEMVVLTCDTPEEDGITWTLDQSSEVLG |
| | SGKTLTIQVKEFGDAGQYTCHKGGEVLSHSLLLLHKKEDGIWSTDILKDQK |
| | EPKNKTFLRCEAKNYSGRFTCWWLTTISTDLTFSVKSSRGSSDPQGVTCGA |
| | ATLSAERVRGDNKEYEYSVECQEDSACPAAEESLPIEVMVDAVHKLKYEN |
| | YTSSFFIRDIIKPDPPKNLQLKPLKNSRQVEVSWEYPDTWSTPHSYFSLTFCV |
| | QVQGKSKREKKDRVFTDKTSATVICRKNASISVRAQDRYYSSSWSEWASV |
| | PCSGGGGSGGGGSGGGGSRNLPVATPDPGMFPCLHHSQNLLRAVSNMLQK |
| | ARQTLEFYPCTSEEIDHEDITKDKTSTVEACLPLELTKNESCLNSRETSFITN |
| | GSCLASRKTSFMMALCLSSIYEDLKMYQVEFKTMNAKLLMDPKRQIFLDQ |
| | NMLAVIDELMQALNFNSETVPQKSSLEEPDFYKTKIKLCILLHAFRIRAVTID |
| | RVMSYLNAS |
| SEQ ID NO: 584 | IWELKKDVYVVELDWYPDAPGEMVVLTCDTPEEDGITWTLDQSSEVLGSG |
| IL12ecd-Fc- | KTLTIQVKEFGDAGQYTCHKGGEVLSHSLLLLHKKEDGIWSTDILKDQKEP |
| SIRPaecd | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMMALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNASASTKGPSVFPLAPCSRSTSESTAALGCLVKDYFPEPVTVSWNSG |
| | ALTSGVHTFPAVLQSSGLYSLSSVVTVPSSSLGTKTYTCNVDHKPSNTKVD |
| | KRVESKYGPPCPSCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQE |
| | GNVFSCSVMHEALHNHYTQKSLSLSLGKGGGGSGGGGSGGGGSEEELQVI |
| | QPDKSVLVAAGETATLRCTATSLIPVGPIQWFRGAGPGRELIYNQKEGHFPR |
| | VTTVSDLTKRNNMDFSIRIGNITPADAGTYYCVKFRKGSPDDVEFKSGAGT |
| | ELSVRAKPS |
| SEQ ID NO: 585 | EEELQVIQPDKSVLVAAGETATLRCTATSLIPVGPIQWFRGAGPGRELIYNQ |
| SIRPaecd-Fc- | KEGHFPRVTTVSDLTKRNNMDFSIRIGNITPADAGTYYCVKFRKGSPDDVE |
| IL15ecd | FKSGAGTELSVRAKPSASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPSCPAPEFLGGPSVFLFPPKPKDTLMISRTPEVT |
| | CVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGKGGGGSGGGGSGGG |
| | GSNWVNVISDLKKIEDLIQSMHIDATLYTESDVHPSCKVTAMKCFLLELQVI |
| | SLESGDASIHDTVENLIILANNSLSSNGNVTESGCKECEELEEKNIKEFLQSF |
| | VHIVQMFINTS |
| SEQ ID NO: 586 | NWVNVISDLKKIEDLIQSMHIDATLYTESDVHPSCKVTAMKCFLLELQVISL |
| IL15ecd-Fc- | ESGDASIHDTVENLIILANNSLSSNGNVTESGCKECEELEEKNIKEFLQSFVH |
| SIRPaecd | IVQMFINTSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKV |
| | DKRVESKYGPPCPSCPAPEFLGGPSVFLFPPKPKDTLMISRTPEVTCVVVDV |
| | SQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQ |
| | EGNVFSCSVMHEALHNHYTQKSLSLSLGKGGGGSGGGGSGGGGSEEELQV |
| | IQPDKSVLVAAGETATLRCTATSLIPVGPIQWFRGAGPGRELIYNQKEGHFP |
| | RVTTVSDLTKRNNMDFSIRIGNITPADAGTYYCVKFRKGSPDDVEFKSGAG |
| | TELSVRAKPS |
| SEQ ID NO: 587 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| IL12ecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSIWELKKDVYVVELDWYPDAPGEMVVLTCDTPE |
| | EDGITWTLDQSSEVLGSGKTLTIQVKEFGDAGQYTCHKGGEVLSHSLLLLH |
| | KKEDGIWSTDILKDQKEPKNKIFLRCEAKNYSGRFTCWWLTTISTDLTFSV |
| | KSSRGSSDPQGVTCGAATLSAERVRGDNKEYEYSVECQEDSACPAAEESLP |
| | IEVMVDAVHKLKYENYTSSFFIRDIIKPDPPKNLQLKPLKNSRQVEVSWEYP |
| | DTWSTPHSYFSLTFCVQVQGKSKREKKDRVFTDKTSATVICRKNASISVRA |
| | QDRYYSSSWSEWASVPCSGGGGSGGGGSGGGGSRNLPVATPDPGMFPCLH |
| | HSQNLLRAVSNMLQKARQTLEFYPCTSEEIDHEDITKDKTSTVEACLPLELT |
| | KNESCLNSRETSFITNGSCLASRKTSFMMALCLSSIYEDLKMYQVEFKTMN |
| | AKLLMDPKRQIFLDQNMLAVIDELMQALNFNSETVPQKSSLEEPDFYKTKI |
| | KLCILLHAFRIRAVTIDRVMSYLNAS |
| SEQ ID NO: 588 | IWELKKDVYVVELDWYPDAPGEMVVLTCDTPEEDGITWTLDQSSEVLGSG |
| IL12ecd-Fc- | KTLTIQVKEFGDAGQYTCHKGGEVLSHSLLLLHKKEDGIWSTDILKDQKEP |
| TGFbRecd | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMMALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNASASTKGPSVFPLAPCSRSTSESTAALGCLVKDYFPEPVTVSWNSG |
| | ALTSGVHTFPAVLQSSGLYSLSSVVTVPSSSLGTKTYTCNVDHKPSNTKVD |
| | KRVESKYGPPCPSCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQE |
| | GNVFSCSVMHEALHNHYTQKSLSLSLGKGGGGSGGGGSGGGGSTIPPHVQ |
| | KSVNNDMIVTDNNGAVKFPQLCKFCDVRFSTCDNQKSCMSNCSITSICEKP |
| | QEVCVAVWRKNDENITLETVCHDPKLPYHDFILEDAASPKCIMKEKKKPGE |
| | TFFMCSCSSDECNDNIIFSEEYNTSNPD |
| SEQ ID NO: 589 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| IL15ecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSNWVNVISDLKKIEDLIQSMHIDATLYTESDVHPS |
| | CKVTAMKCFLLELQVISLESGDASIHDTVENLIILANNSLSSNGNVTESGCK |
| | ECEELEEKNIKEFLQSFVHIVQMFINTS |
| SEQ ID NO: 590 | NWVNVISDLKKIEDLIQSMHIDATLYTESDVHPSCKVTAMKCFLLELQVISL |
| IL15ecd-Fc- | ESGDASIHDTVENLIILANNSLSSNGNVTESGCKECEELEEKNIKEFLQSFVH |
| TGFbRecd | IVQMFINTSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKV |
| | DKRVESKYGPPCPSCPAPEFLGGPSVFLFPPKPKDTLMISRTPEVTCVVVDV |
| | SQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQ |
| | EGNVFSCSVMHEALHNHYTQKSLSLSLGKGGGGSGGGGSGGGGSTIPPHV |
| | QKSVNNDMIVTDNNGAVKFPQLCKFCDVRFSTCDNQKSCMSNCSITSICEK |
| | PQEVCVAVWRKNDENITLETVCHDPKLPYHDFILEDAASPKCIMKEKKKPG |
| | ETFFMCSCSSDECNDNIIFSEEYNTSNPD |
| SEQ ID NO: 591 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| IL12ecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPCSRSTSESTAA |
| | LGCLVKDYFPEPVTVSWNSGALTSGVHTFPAVLQSSGLYSLSSVVTVPSSSL |
| | GTKTYTCNVDHKPSNTKVDKRVESKYGPPCPSCPAPEFLGGPSVFLFPPKPK |
| | DTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGKGG |
| | GGSGGGGSGGGGSIWELKKDVYVVELDWYPDAPGEMVVLTCDTPEEDGI |
| | TWTLDQSSEVLGSGKTLTIQVKEFGDAGQYTCHKGGEVLSHSLLLLHKKE |
| | DGIWSTDILKDQKEPKNKTFLRCEAKNYSGRFTCWWLTTISTDLTFSVKSS |
| | RGSSDPQGVTCGAATLSAERVRGDNKEYEYSVECQEDSACPAAEESLPIEV |
| | MVDAVHKLKYENYTSSFFIRDIIKPDPPKNLQLKPLKNSRQVEVSWEYPDT |
| | WSTPHSYFSLTFCVQVQGKSKREKKDRVFTDKTSATVICRKNASISVRAQD |
| | RYYSSSWSEWASVPCSGGGGSGGGGSGGGGSRNLPVATPDPGMFPCLHHS |
| | QNLLRAVSNMLQKARQTLEFYPCTSEEIDHEDITKDKTSTVEACLPLELTKN |
| | ESCLNSRETSFITNGSCLASRKTSFMMALCLSSIYEDLKMYQVEFKTMNAK |
| | LLMDPKRQIFLDQNMLAVIDELMQALNFNSETVPQKSSLEEPDFYKTKIKL |
| | CILLHAFRIRAVTIDRVMSYLNAS |
| SEQ ID NO: 592 | IWELKKDVYVVELDWYPDAPGEMVVLTCDTPEEDGITWTLDQSSEVLGSG |
| IL12ecd-Fc- | KTLTIQVKEFGDAGQYTCHKGGEVLSHSLLLLHKKEDGIWSTDILKDQKEP |
| TIM3ecd | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMMALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNASASTKGPSVFPLAPCSRSTSESTAALGCLVKDYFPEPVTVSWNSG |
| | ALTSGVHTFPAVLQSSGLYSLSSVVTVPSSSLGTKTYTCNVDHKPSNTKVD |
| | KRVESKYGPPCPSCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQE |
| | GNVFSCSVMHEALHNHYTQKSLSLSLGKGGGGSGGGGSGGGGSSEVEYRA |
| | EVGQNAYLPCFYTPAAPGNLVPVCWGKGACPVFECGNVVLRTDERDVNY |
| | WTSRYWLNGDFRKGDVSLTIENVTLADSGIYCCRIQIPGIMNDEKFNLKLVI |
| | KPAKVTPAPTRQRDFTAAFPRMLTTRGHGPAETQTLGSLPDINLTQISTLAN |
| | ELRDSRLANDLRDSGATIRIG |
| SEQ ID NO: 593 | SEVEYRAEVGQNAYLPCFYTPAAPGNLVPVCWGKGACPVFECGNVVLRT |
| TIM3ecd-Fc- | DERDVNYWTSRYWLNGDFRKGDVSLTIENVTLADSGIYCCRIQIPGIMNDE |
| IL15ecd | KFNLKLVIKPAKVTPAPTRQRDFTAAFPRMLTTRGHGPAETQTLGSLPDINL |
| | TQISTLANELRDSRLANDLRDSGATIRIGASTKGPSVFPLAPCSRSTSESTAA |
| | LGCLVKDYFPEPVTVSWNSGALTSGVHTFPAVLQSSGLYSLSSVVTVPSSSL |
| | GTKTYTCNVDHKPSNTKVDKRVESKYGPPCPSCPAPEFLGGPSVFLFPPKPK |
| | DTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGKGG |
| | GGSGGGGSGGGGSNWVNVISDLKKIEDLIQSMHIDATLYTESDVHPSCKVT |
| | AMKCFLLELQVISLESGDASIHDTVENLIILANNSLSSNGNVTESGCKECEEL |
| | EEKNIKEFLQSFVHIVQMFINTS |
| SEQ ID NO: 594 | NWVNVISDLKKIEDLIQSMHIDATLYTESDVHPSCKVTAMKCFLLELQVISL |
| IL15ecd-Fc- | ESGDASIHDTVENLIILANNSLSSNGNVTESGCKECEELEEKNIKEFLQSFVH |
| TIM3ecd | IVQMFINTSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKV |
| | DKRVESKYGPPCPSCPAPEFLGGPSVFLFPPKPKDTLMISRTPEVTCVVVDV |
| | SQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQ |
| | EGNVFSCSVMHEALHNHYTQKSLSLSLGKGGGGSGGGGSGGGGSSEVEYR |
| | AEVGQNAYLPCFYTPAAPGNLVPVCWGKGACPVFECGNVVLRTDERDVN |
| | YWTSRYWLNGDFRKGDVSLTIENVTLADSGIYCCRIQIPGIMNDEKFNLKL |
| | VIKPAKVTPAPTRQRDFTAAFPRMLTTRGHGPAETQTLGSLPDINLTQISTL |
| | ANELRDSRLANDLRDSGATIRIG |
| SEQ ID NO: 595 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| IL12ecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHIFPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSIWELKKDVYVVELDWYP |
| | DAPGEMVVLTCDTPEEDGITWTLDQSSEVLGSGKTLTIQVKEFGDAGQYTC |
| | HKGGEVLSHSLLLLHKKEDGIWSTDILKDQKEPKNKTFLRCEAKNYSGRFT |
| | CWWLTTISTDLTFSVKSSRGSSDPQGVTCGAATLSAERVRGDNKEYEYSVE |
| | CQEDSACPAAEESLPIEVMVDAVHKLKYENYTSSFFIRDIIKPDPPKNLQLKP |
| | LKNSRQVEVSWEYPDTWSTPHSYFSLTFCVQVQGKSKREKKDRVFTDKTS |
| | ATVICRKNASISVRAQDRYYSSSWSEWASVPCSGGGGSGGGGSGGGGSRN |
| | LPVATPDPGMFPCLHHSQNLLRAVSNMLQKARQTLEFYPCTSEEIDHEDIT |
| | KDKTSTVEACLPLELTKNESCLNSRETSFITNGSCLASRKTSFMMALCLSSIY |
| | EDLKMYQVEFKTMNAKLLMDPKRQIFLDQNMLAVIDELMQALNFNSETV |
| | PQKSSLEEPDFYKTKIKLCILLHAFRIRAVTIDRVMSYLNAS |
| SEQ ID NO: 596 | IWELKKDVYVVELDWYPDAPGEMVVLTCDTPEEDGITWTLDQSSEVLGSG |
| IL12ecd-Fc- | KTLTIQVKEFGDAGQYTCHKGGEVLSHSLLLLHKKEDGIWSTDILKDQKEP |
| VEGFRecd | KNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYT |
| | SSFFIRDIIKPDPPKNLQLKPLKNSRQVEVSWEYPDTWSTPHSYFSLTFCVQV |
| | QGKSKREKKDRVFTDKTSATVICRKNASISVRAQDRYYSSSWSEWASVPCS |
| | GGGGSGGGGSGGGGSRNLPVATPDPGMFPCLHHSQNLLRAVSNMLQKAR |
| | QTLEFYPCTSEEIDHEDITKDKTSTVEACLPLELTKNESCLNSRETSFITNGSC |
| | LASRKTSFMNIALCLSSIYEDLKMYQVEFKTMNAKLLMDPKRQIFLDQNML |
| | AVIDELMQALNFNSETVPQKSSLEEPDFYKTKIKLCILLHAFRIRAVTIDRV |
| | MSYLNASASTKGPSVFPLAPCSRSTSESTAALGCLVKDYFPEPVTVSWNSG |
| | ALTSGVHTFPAVLQSSGLYSLSSVVTVPSSSLGTKTYTCNVDHKPSNTKVD |
| | KRVESKYGPPCPSCPAPEFLGGPSVFLFPPKPKDTLMISRTPEVTCVVVDVS |
| | QEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQE |
| | GNVFSCSVMHEALHNHYTQKSLSLSLGKGGGGSGGGGSGGGGSSDTGRPF |
| | VEMYSEIPEIIHMTEGRELVIPCRVTSPNITVTLKKFPLDTLIPDGKRIIWDSR |
| | KGFIISNATYKEIGLLTCEATVNGHLYKTNYLTHRQTNTIIDVVLSPSHGIEL |
| | SVGEKLVLNCTARTELNVGIDFNWEYPSSKHQHKKLVNRDLKTQSGSEMK |
| | KFLSTLTIDGVTRSDQGLYTCAASSGLMTKKNSTFVRVHEK |
| SEQ ID NO: 597 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| IL15ecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSNWVNVISDLKKIEDLIQS |
| | MHIDATLYTESDVHPSCKVTAMKCFLLELQVISLESGDASIHDTVENLIILA |
| | NNSLSSNGNVTESGCKECEELEEKNIKEFLQSFVHIVQMFINTS |
| SEQ ID NO: 598 | NWVNVISDLKKIEDLIQSMHIDATLYTESDVHPSCKVTAMKCFLLELQVISL |
| IL15ecd-Fc- | ESGDASIHDTVENLIILANNSLSSNGNVTESGCKECEELEEKNIKEFLQSFVH |
| VEGFRecd | IVQMFINTSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKV |
| | DKRVESKYGPPCPSCPAPEFLGGPSVFLFPPKPKDTLMISRTPEVTCVVVDV |
| | SQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQ |
| | EGNVFSCSVMHEALHNHYTQKSLSLSLGKGGGGSGGGGSGGGGSSDTGRP |
| | FVEMYSEIPEIIHMTEGRELVIPCRVTSPNITVTLKKFPLDTLIPDGKRIIWDS |
| | RKGFIISNATYKEIGLLTCEATVNGHLYKTNYLTHRQTNTIIDVVLSPSHGIE |
| | LSVGEKLVLNCTARTELNVGIDFNWEYPSSKHQHKKLVNRDLKTQSGSEM |
| | KKFLSTLTIDGVTRSDQGLYTCAASSGLMTKKNSTFVRVHEK |
| SEQ ID NO: 599 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-41BBLecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSACPWAVSGARASPGSAASPRLREGPELSPDDPAGLLDLRQGM |
| | FAQLVAQNVLLIDGPLSWYSDPGLAGVSLTGGLSYKEDTKELVVAKAGVY |
| | YVFFQLELRRVVAGEGSGSVSLALHLQPLRSAAGAAALALTVDLPPASSEA |
| | RNSAFGFQGRLLHLSAGQRLGVHLHTEARARHAWQLTQGATVLGLFRVTP |
| | EIPAGLPSPRSE |
| SEQ ID NO: 600 | ACPWAVSGARASPGSAASPRLREGPELSPDDPAGLLDLRQGMFAQLVAQN |
| 41BBLecd-Fc- | VLLIDGPLSWYSDPGLAGVSLTGGLSYKEDTKELVVAKAGVYYVFFQLEL |
| SIGLEC10ecd | RRVVAGEGSGSVSLALHLQPLRSAAGAAALALTVDLPPASSEARNSAFGFQ |
| | GRLLHLSAGQRLGVHLHTEARARHAWQLTQGATVLGLFRVTPEIPAGLPSP |
| | RSEASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVES |
| | KYGPPCPSCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGKEYK |
| | CKVSNKGLPSSIEKTISKAKGQPREPQVYTLPPSQEEMTKNQVSLTCLVKGF |
| | YPSDIAVEWESNGQPENNYKTTPPVLDSDGSFFLYSRLTVDKSRWQEGNVF |
| | SCSVMHEALHNHYTQKSLSLSLGKGGGGSGGGGSGGGGSMDGRFWIRVQ |
| | ESVMVPEGLCISVPCSFSYPRQDWTGSTPAYGYWFKAVTETTKGAPVATN |
| | HQSREVEMSTRGRFQLTGDPAKGNCSLVIRDAQMQDESQYFFRVERGSYV |
| | RYNFMNDGFFLKVTALTQKPDVYIPETLEPGQPVTVICVFNWAFEECPPPSF |
| | SWTGAALSSQGTKPTTSHFSVLSFTPRPQDHNTDLTCHVDFSRKGVSAQRT |
| | VRLRVAYAPRDLVISISRDNTPALEPQPQGNVPYLEAQKGQFLRLLCAADS |
| | QPPATLSWVLQNRVLSSSHPWGPRPLGLELPGVKAGDSGRYTCRAENRLG |
| | SQQRALDLSVQYPPENLRVMVSQANRTVLENLGNGTSLPVLEGQSLCLVC |
| | VTHSSPPARLSWTQRGQVLSPSQPSDPGVLELPRVQVEHEGEFTCHARHPL |
| | GSQHVSLSLSVHYSPKLLGPSCSWEAEGLHCSCSSQASPAPSLRWWLGEEL |
| | LEGNSSQDSFEVTPSSAGPWANSSLSLHGGLSSGLRLRCEAWNVHGAQSGS |
| | ILQLPDKKGLIST |
| SEQ ID NO: 601 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-CD30Lecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSQRTDSIPNSPDNVPLKGGNCSEDLLCILKRAPFKKSWAYLQVA |
| | KHLNKTKLSWNKDGILHGVRYQDGNLVIQFPGLYFIICQLQFLVQCPNNSV |
| | DLKLELLINKHIKKQALVTVCESGMQTKHVYQNLSQFLLDYLQVNTTISVN |
| | VDTFQYIDTSTFPLENVLSIFLYSNSD |
| SEQ ID NO: 602 | QRTDSIPNSPDNVPLKGGNCSEDLLCILKRAPFKKSWAYLQVAKHLNKTKL |
| CD30Lecd-Fc- | SWNKDGILHGVRYQDGNLVIQFPGLYFIICQLQFLVQCPNNSVDLKLELLIN |
| SIGLEC10ecd | KHIKKQALVTVCESGMQTKHVYQNLSQFLLDYLQVNTTISVNVDTFQYIDT |
| | STFPLENVLSIFLYSNSDASTKGPSVFPLAPCSRSTSESTAALGCLVKDYFPE |
| | PVTVSWNSGALTSGVHTFPAVLQSSGLYSLSSVVTVPSSSLGTKTYTCNVD |
| | HKPSNTKVDKRVESKYGPPCPSCPAPEFLGGPSVFLFPPKPKDTLMISRTPE |
| | VTCVVVDVSQEDPEVQFNWYVDGVEVHNAKTKPREEQFNSTYRVVSVLT |
| | VLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSR |
| | LTVDKSRWQEGNVFSCSVMHEALHNHYTQKSLSLSLGKGGGGSGGGGSG |
| | GGGSMDGRFWIRVQESVMVPEGLCISVPCSFSYPRQDWTGSTPAYGYWFK |
| | AVTETTKGAPVATNHQSREVEMSTRGRFQLTGDPAKGNCSLVIRDAQMQD |
| | ESQYFFRVERGSYVRYNFMNDGFFLKVTALTQKPDVYIPETLEPGQPVTVI |
| | CVFNWAFEECPPPSFSWTGAALSSQGTKPTTSHFSVLSFTPRPQDHNTDLTC |
| | HVDFSRKGVSAQRTVRLRVAYAPRDLVISISRDNTPALEPQPQGNVPYLEA |
| | QKGQFLRLLCAADSQPPATLSWVLQNRVLSSSHPWGPRPLGLELPGVKAG |
| | DSGRYTCRAENRLGSQQRALDLSVQYPPENLRVMVSQANRTVLENLGNGT |
| | SLPVLEGQSLCLVCVTHSSPPARLSWTQRGQVLSPSQPSDPGVLELPRVQVE |
| | HEGEFTCHARHPLGSQHVSLSLSVHYSPKLLGPSCSWEAEGLHCSCSSQASP |
| | APSLRWWLGEELLEGNSSQDSFEVTPSSAGPWANSSLSLHGGLSSGLRLRC |
| | EAWNVHGAQSGSILQLPDKKGLIST |
| SEQ ID NO: 603 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-CD40Lecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSGDQNPQIAAHVISEASSKTTSVLQWAEKGYYTMSNNLVTLEN |
| | GKQLTVKRQGLYYIYAQVTFCSNREASSQAPFIASLCLKSPGRFERILLRAA |
| | NTHSSAKPCGQQSIHLGGVFELQPGASVFVNVTDPSQVSHGTGFTSFGLLK |
| | L |
| SEQ ID NO: 604 | GDQNPQIAAHVISEASSKTTSVLQWAEKGYYTMSNNLVTLENGKQLTVKR |
| CD40Lecd-Fc- | QGLYYIYAQVTFCSNREASSQAPFIASLCLKSPGRFERILLRAANTHSSAKPC |
| SIGLEC10ecd | GQQSIHLGGVFELQPGASVFVNVTDPSQVSHGTGFTSFGLLKLASTKGPSVF |
| | PLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPSCPAPE |
| | FLGGPSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIAVEWESNG |
| | QPENNYKTTPPVLDSDGSFFLYSRLTVDKSRWQEGNVFSCSVMHEALHNH |
| | YTQKSLSLSLGKGGGGSGGGGSGGGGSMDGRFWIRVQESVMVPEGLCISV |
| | PCSFSYPRQDWTGSTPAYGYWFKAVTETTKGAPVATNHQSREVEMSTRGR |
| | FQLTGDPAKGNCSLVIRDAQMQDESQYFFRVERGSYVRYNFMNDGFFLKV |
| | TALTQKPDVYIPETLEPGQPVTVICVFNWAFEECPPPSFSWTGAALSSQGTK |
| | PTTSHFSVLSFTPRPQDHNTDLTCHVDFSRKGVSAQRTVRLRVAYAPRDLV |
| | ISISRDNTPALEPQPQGNVPYLEAQKGQFLRLLCAADSQPPATLSWVLQNR |
| | VLSSSHPWGPRPLGLELPGVKAGDSGRYTCRAENRLGSQQRALDLSVQYPP |
| | ENLRVMVSQANRTVLENLGNGTSLPVLEGQSLCLVCVTHSSPPARLSWTQ |
| | RGQVLSPSQPSDPGVLELPRVQVEHEGEFTCHARHPLGSQHVSLSLSVHYSP |
| | KLLGPSCSWEAEGLHCSCSSQASPAPSLRWWLGEELLEGNSSQDSFEVTPSS |
| | AGPWANSSLSLHGGLSSGLRLRCEAWNVHGAQSGSILQLPDKKGLIST |
| SEQ ID NO: 605 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-CD70ecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSQRFAQAQQQLPLESLGWDVAELQLNHTGPQQDPRLYWQGGP |
| | ALGRSFLHGPELDKGQLRIHRDGIYMVHIQVTLAICSSTTASRHHPTTLAVG |
| | ICSPASRSISLLRLSFHQGCTIASQRLTPLARGDTLCTNLTGTLLPSRNTDETF |
| | FGVQWVRP |
| SEQ ID NO: 606 | QRFAQAQQQLPLESLGWDVAELQLNHTGPQQDPRLYWQGGPALGRSFLH |
| CD70ecd-Fc- | GPELDKGQLRIHRDGIYMVHIQVTLAICSSTTASRHHPTTLAVGICSPASRSI |
| SIGLEC10ecd | SLLRLSFHQGCTIASQRLTPLARGDTLCTNLTGTLLPSRNTDETFFGVQWVR |
| | PASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKY |
| | GPPCPSCPAPEFLGGPSVFLFPPKPKDTLMISRTPEVTCVVVDVSQEDPEVQF |
| | NWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSCS |
| | VMHEALHNHYTQKSLSLSLGKGGGGSGGGGSGGGGSMDGRFWIRVQESV |
| | MVPEGLCISVPCSFSYPRQDWTGSTPAYGYWFKAVTETTKGAPVATNHQS |
| | REVEMSTRGRFQLTGDPAKGNCSLVIRDAQMQDESQYFFRVERGSYVRYN |
| | FMNDGFFLKVTALTQKPDVYIPETLEPGQPVTVICVFNWAFEECPPPSFSWT |
| | GAALSSQGTKPTTSHFSVLSFTPRPQDHNTDLTCHVDFSRKGVSAQRTVRL |
| | RVAYAPRDLVISISRDNTPALEPQPQGNVPYLEAQKGQFLRLLCAADSQPP |
| | ATLSWVLQNRVLSSSHPWGPRPLGLELPGVKAGDSGRYTCRAENRLGSQQ |
| | RALDLSVQYPPENLRVMVSQANRTVLENLGNGTSLPVLEGQSLCLVCVTH |
| | SSPPARLSWTQRGQVLSPSQPSDPGVLELPRVQVEHEGEFTCHARHPLGSQ |
| | HVSLSLSVHYSPKLLGPSCSWEAEGLHCSCSSQASPAPSLRWWLGEELLEG |
| | NSSQDSFEVTPSSAGPWANSSLSLHGGLSSGLRLRCEAWNVHGAQSGSILQ |
| | LPDKKGLIST |
| SEQ ID NO: 607 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-GITRLecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSQLETAKEPCMAKFGPLPSKWQMASSEPPCVNKVSDWKLEILQ |
| | NGLYLIYGQVAPNANYNDVAPFEVRLYKNKDMIQTLTNKSKIQNVGGTYE |
| | LHVGDTIDLIFNSEHQVLKNNTYWGIILLANPQFIS |
| SEQ ID NO: 608 | QLETAKEPCMAKFGPLPSKWQMASSEPPCVNKVSDWKLEILQNGLYLIYG |
| GITRLecd-Fc- | QVAPNANYNDVAPFEVRLYKNKDMIQTLTNKSKIQNVGGTYELHVGDTID |
| SIGLEC10ecd | LIFNSEHQVLKNNTYWGIILLANPQFISASTKGPSVFPLAPCSRSTSESTAAL |
| | GCLVKDYFPEPVTVSWNSGALTSGVHTFPAVLQSSGLYSLSSVVTVPSSSL |
| | GTKTYTCNVDHKPSNTKVDKRVESKYGPPCPSCPAPEFLGGPSVFLFPPKPK |
| | DTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGKGG |
| | GGSGGGGSGGGGSMDGRFWIRVQESVMVPEGLCISVPCSFSYPRQDWTGS |
| | TPAYGYWFKAVTETTKGAPVATNHQSREVEMSTRGRFQLTGDPAKGNCSL |
| | VIRDAQMQDESQYFFRVERGSYVRYNFMNDGFFLKVTALTQKPDVYIPET |
| | LEPGQPVTVICVFNWAFEECPPPSFSWTGAALSSQGTKPTTSHFSVLSFTPRP |
| | QDHNTDLTCHVDFSRKGVSAQRTVRLRVAYAPRDLVISISRDNTPALEPQP |
| | QGNVPYLEAQKGQFLRLLCAADSQPPATLSWVLQNRVLSSSHPWGPRPLG |
| | LELPGVKAGDSGRYTCRAENRLGSQQRALDLSVQYPPENLRVMVSQANRT |
| | VLENLGNGTSLPVLEGQSLCLVCVTHSSPPARLSWTQRGQVLSPSQPSDPG |
| | VLELPRVQVEHEGEFTCHARHPLGSQHVSLSLSVHYSPKLLGPSCSWEAEG |
| | LHCSCSSQASPAPSLRWWLGEELLEGNSSQDSFEVTPSSAGPWANSSLSLH |
| | GGLSSGLRLRCEAWNVHGAQSGSILQLPDKKGLIST |
| SEQ ID NO: 609 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-ICOSLecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSDTQEKEVRAMVGSDVELSCACPEGSRFDLNDVYVYWQTSES |
| | KTVVTYHIPQNSSLENVDSRYRNRALMSPAGMLRGDFSLRLFNVTPQDEQ |
| | KFHCLVLSQSLGFQEVLSVEVTLHVAANFSVPVVSAPHSPSQDELTFTCTSI |
| | NGYPRPNVYWINKTDNSLLDQALQNDTVFLNMRGLYDVVSVLRIARTPSV |
| | NIGCCIENVLLQQNLTVGSQTGNDIGERDKITENPVSTGEKNAATWS |
| SEQ ID NO: 610 | DTQEKEVRAMVGSDVELSCACPEGSRFDLNDVYVYWQTSESKTVVTYHIP |
| ICOSLecd-Fc- | QNSSLENVDSRYRNRALMSPAGMLRGDFSLRLFNVTPQDEQKFHCLVLSQ |
| SIGLEC10ec | SLGFQEVLSVEVTLHVAANFSVPVVSAPHSPSQDELTFTCTSINGYPRPNVY |
| | WINKTDNSLLDQALQNDTVFLNMRGLYDVVSVLRIARTPSVNIGCCIENVL |
| | LQQNLTVGSQTGNDIGERDKITENPVSTGEKNAATWSASTKGPSVFPLAPC |
| | SRSTSESTAALGCLVKDYFPEPVTVSWNSGALTSGVHITPAVLQSSGLYSLS |
| | SVVTVPSSSLGTKTYTCNVDHKPSNTKVDKRVESKYGPPCPSCPAPEFLGG |
| | PSVFLFPPKPKDTLMISRTPEVTCVVVDVSQEDPEVQFNWYVDGVEVHNA |
| | KTKPREEQFNSTYRVVSVLTVLHQDWLNGKEYKCKVSNKGLPSSIEKTISK |
| | AKGQPREPQVYTLPPSQEEMTKNQVSLTCLVKGFYPSDIAVEWESNGQPEN |
| | NYKTTPPVLDSDGSFFLYSRLTVDKSRWQEGNVFSCSVMHEALHNHYTQK |
| | SLSLSLGKGGGGSGGGGSGGGGSMDGRFWIRVQESVMVPEGLCISVPCSFS |
| | YPRQDWTGSTPAYGYWFKAVTETTKGAPVATNHQSREVEMSTRGRFQLT |
| | GDPAKGNCSLVIRDAQMQDESQYFFRVERGSYVRYNFMNDGFFLKVTALT |
| | QKPDVYIPETLEPGQPVTVICVFNWAFEECPPPSFSWTGAALSSQGTKPTTS |
| | HFSVLSFTPRPQDHNTDLTCHVDFSRKGVSAQRTVRLRVAYAPRDLVISISR |
| | DNTPALEPQPQGNVPYLEAQKGQFLRLLCAADSQPPATLSWVLQNRVLSSS |
| | HPWGPRPLGLELPGVKAGDSGRYTCRAENRLGSQQRALDLSVQYPPENLR |
| | VMVSQANRTVLENLGNGTSLPVLEGQSLCLVCVTHSSPPARLSWTQRGQV |
| | LSPSQPSDPGVLELPRVQVEHEGEFTCHARHPLGSQHVSLSLSVHYSPKLLG |
| | PSCSWEAEGLHCSCSSQASPAPSLRWWLGEELLEGNSSQDSFEVTPSSAGP |
| | WANSSLSLHGGLSSGLRLRCEAWNVHGAQSGSILQLPDKKGLIST |
| SEQ ID NO: 611 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-LIGHTecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSDGPAGSWEQLIQERRSHEVNPAAHLTGANSSLTGSGGPLLWE |
| | TQLGLAFLRGLSYHDGALVVTKAGYYYIYSKVQLGGVGCPLGLASTITHG |
| | LYKRTPRYPEELELLVSQQSPCGRATSSSRVWWDSSFLGGVVHLEAGEKV |
| | VVRVLDERLVRLRDGTRSYFGAFMV |
| SEQ ID NO: 612 | DGPAGSWEQLIQERRSHEVNPAAHLTGANSSLTGSGGPLLWETQLGLAFLR |
| LIGHTecd-Fc- | GLSYHDGALVVTKAGYYYIYSKVQLGGVGCPLGLASTITHGLYKRTPRYP |
| SIGLEC10ecd | EELELLVSQQSPCGRATSSSRVWWDSSFLGGVVHLEAGEKVVVRVLDERL |
| | VRLRDGTRSYFGAFMVASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPSCPAPEFLGGPSVFLFPPKPKDTLMISRTPEVT |
| | CVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGKGGGGSGGGGSGGG |
| | GSMDGRFWIRVQESVMVPEGLCISVPCSFSYPRQDWTGSTPAYGYWFKAV |
| | TETTKGAPVATNHQSREVEMSTRGRFQLTGDPAKGNCSLVIRDAQMQDES |
| | QYFFRVERGSYVRYNFMNDGFFLKVTALTQKPDVYIPETLEPGQPVTVICV |
| | FNWAFEECPPPSFSWTGAALSSQGTKPTTSHFSVLSFTPRPQDHNTDLTCHV |
| | DFSRKGVSAQRTVRLRVAYAPRDLVISISRDNTPALEPQPQGNVPYLEAQK |
| | GQFLRLLCAADSQPPATLSWVLQNRVLSSSHPWGPRPLGLELPGVKAGDS |
| | GRYTCRAENRLGSQQRALDLSVQYPPENLRVMVSQANRTVLENLGNGTSL |
| | PVLEGQSLCLVCVTHSSPPARLSWTQRGQVLSPSQPSDPGVLELPRVQVEH |
| | EGEFTCHARHPLGSQHVSLSLSVHYSPKLLGPSCSWEAEGLHCSCSSQASPA |
| | PSLRWWLGEELLEGNSSQDSFEVTPSSAGPWANSSLSLHGGLSSGLRLRCE |
| | AWNVHGAQSGSILQLPDKKGLIST |
| SEQ ID NO: 613 | MDGRFWIRVQESVMVPEGLCISVPCSFSYPRQDWTGSTPAYGYWFKAVTE |
| SIGLEC10ecd- | TTKGAPVATNHQSREVEMSTRGRFQLTGDPAKGNCSLVIRDAQMQDESQY |
| Fc-OX40Lecd | FFRVERGSYVRYNFMNDGFFLKVTALTQKPDVYIPETLEPGQPVTVICVFN |
| | WAFEECPPPSFSWTGAALSSQGTKPTTSHFSVLSFTPRPQDHNTDLTCHVDF |
| | SRKGVSAQRTVRLRVAYAPRDLVISISRDNTPALEPQPQGNVPYLEAQKGQ |
| | FLRLLCAADSQPPATLSWVLQNRVLSSSHPWGPRPLGLELPGVKAGDSGRY |
| | TCRAENRLGSQQRALDLSVQYPPENLRVMVSQANRTVLENLGNGTSLPVL |
| | EGQSLCLVCVTHSSPPARLSWTQRGQVLSPSQPSDPGVLELPRVQVEHEGE |
| | FTCHARHPLGSQHVSLSLSVHYSPKLLGPSCSWEAEGLHCSCSSQASPAPSL |
| | RWWLGEELLEGNSSQDSFEVTPSSAGPWANSSLSLHGGLSSGLRLRCEAW |
| | NVHGAQSGSILQLPDKKGLISTASTKGPSVFPLAPCSRSTSESTAALGCLVK |
| | DYFPEPVTVSWNSGALTSGVHTFPAVLQSSGLYSLSSVVTVPSSSLGTKTYT |
| | CNVDHKPSNTKVDKRVESKYGPPCPSCPAPEFLGGPSVFLFPPKPKDTLMIS |
| | RTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLGKGGGGSGG |
| | GGSGGGGSQVSHRYPRIQSIKVQFTEYKKEKGFILTSQKEDEIMKVQNNSVI |
| | INCDGFYLISLKGYFSQEVNISLHYQKDEEPLFQLKKVRSVNSLMVASLTYK |
| | DKVYLNVTTDNTSLDDFHVNGGELILIHQNPGEFCVL |
| SEQ ID NO: 614 | QVSHRYPRIQSIKVQFTEYKKEKGFILTSQKEDEIMKVQNNSVIINCDGFYLI |
| OX40Lecd-Fc- | SLKGYFSQEVNISLHYQKDEEPLFQLKKVRSVNSLMVASLTYKDKVYLNV |
| SIGLEC10ecd | TTDNTSLDDFHVNGGELILIHQNPGEFCVLASTKGPSVFPLAPCSRSTSESTA |
| | ALGCLVKDYFPEPVTVSWNSGALTSGVHTFPAVLQSSGLYSLSSVVTVPSS |
| | SLGTKTYTCNVDHKPSNTKVDKRVESKYGPPCPSCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGKG |
| | GGGSGGGGSGGGGSMDGRFWIRVQESVMVPEGLCISVPCSFSYPRQDWTG |
| | STPAYGYWFKAVTETTKGAPVATNHQSREVEMSTRGRFQLTGDPAKGNCS |
| | LVIRDAQMQDESQYFFRVERGSYVRYNFMNDGFFLKVTALTQKPDVYIPE |
| | TLEPGQPVTVICVFNWAFEECPPPSFSWTGAALSSQGTKPTTSHFSVLSFTPR |
| | PQDHNTDLTCHVDFSRKGVSAQRTVRLRVAYAPRDLVISISRDNTPALEPQ |
| | PQGNVPYLEAQKGQFLRLLCAADSQPPATLSWVLQNRVLSSSHPWGPRPL |
| | GLELPGVKAGDSGRYTCRAENRLGSQQRALDLSVQYPPENLRVMVSQANR |
| | TVLENLGNGTSLPVLEGQSLCLVCVTHSSPPARLSWTQRGQVLSPSQPSDP |
| | GVLELPRVQVEHEGEFTCHARHPLGSQHVSLSLSVHYSPKLLGPSCSWEAE |
| | GLHCSCSSQASPAPSLRWWLGEELLEGNSSQDSFEVTPSSAGPWANSSLSL |
| | HGGLSSGLRLRCEAWNVHGAQSGSILQLPDKKGLIST |
| SEQ ID NO: 615 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| 41BBLecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSACPWAVSGARASPGSAASPRLREGPELSPDDPA |
| | GLLDLRQGMFAQLVAQNVLLIDGPLSWYSDPGLAGVSLTGGLSYKEDTKE |
| | LVVAKAGVYYVFFQLELRRVVAGEGSGSVSLALHLQPLRSAAGAAALALT |
| | VDLPPASSEARNSAFGFQGRLLHLSAGQRLGVHLHTEARARHAWQLTQGA |
| | TVLGLFRVTPEIPAGLPSPRSE |
| SEQ ID NO: 616 | ACPWAVSGARASPGSAASPRLREGPELSPDDPAGLLDLRQGMFAQLVAQN |
| 41BBLecd-Fc- | VLLIDGPLSWYSDPGLAGVSLTGGLSYKEDTKELVVAKAGVYYVFFQLEL |
| TGFbRecd | RRVVAGEGSGSVSLALHLQPLRSAAGAAALALTVDLPPASSEARNSAFGFQ |
| | GRLLHLSAGQRLGVHLHTEARARHAWQLTQGATVLGLFRVTPEIPAGLPSP |
| | RSEASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVES |
| | KYGPPCPSCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGKEYK |
| | CKVSNKGLPSSIEKTISKAKGQPREPQVYTLPPSQEEMTKNQVSLTCLVKGF |
| | YPSDIAVEWESNGQPENNYKTTPPVLDSDGSFFLYSRLTVDKSRWQEGNVF |
| | SCSVMHEALHNHYTQKSLSLSLGKGGGGSGGGGSGGGGSTIPPHVQKSVN |
| | NDMIVTDNNGAVKFPQLCKFCDVRFSTCDNQKSCMSNCSITSICEKPQEVC |
| | VAVWRKNDENITLETVCHDPKLPYHDFILEDAASPKCIMKEKKKPGETFFM |
| | CSCSSDECNDNIIFSEEYNTSNPD |
| SEQ ID NO: 617 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| CD30Lecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSQRTDSIPNSPDNVPLKGGNCSEDLLCILKRAPFK |
| | KSWAYLQVAKHLNKTKLSWNKDGILHGVRYQDGNLVIQFPGLYFIICQLQ |
| | FLVQCPNNSVDLKLELLINKHIKKQALVTVCESGMQTKHVYQNLSQFLLD |
| | YLQVNTTISVNVDTFQYIDTSTFPLENVLSIFLYSNSD |
| SEQ ID NO: 618 | QRTDSIPNSPDNVPLKGGNCSEDLLCILKRAPFKKSWAYLQVAKHLNKTKL |
| CD30Lecd-Fc- | SWNKDGILHGVRYQDGNLVIQFPGLYFIICQLQFLVQCPNNSVDLKLELLIN |
| TGFbRecd | KHIKKQALVTVCESGMQTKHVYQNLSQFLLDYLQVNTTISVNVDTFQYIDT |
| | STFPLENVLSIFLYSNSDASTKGPSVFPLAPCSRSTSESTAALGCLVKDYFPE |
| | PVTVSWNSGALTSGVHTFPAVLQSSGLYSLSSVVTVPSSSLGTKTYTCNVD |
| | HKPSNTKVDKRVESKYGPPCPSCPAPEFLGGPSVFLFPPKPKDTLMISRTPE |
| | VTCVVVDVSQEDPEVQFNWYVDGVEVHNAKTKPREEQFNSTYRVVSVLT |
| | VLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSR |
| | LTVDKSRWQEGNVFSCSVMHEALHNHYTQKSLSLSLGKGGGGSGGGGSG |
| | GGGSTIPPHVQKSVNNDMIVTDNNGAVKFPQLCKFCDVRFSTCDNQKSCM |
| | SNCSITSICEKPQEVCVAVWRKNDENITLETVCHDPKLPYHDFILEDAASPK |
| | CIMKEKKKPGETFFMCSCSSDECNDNIIFSEEYNTSNPD |
| SEQ ID NO: 619 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| CD40Lecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSGDQNPQIAAHVISEASSKTTSVLQWAEKGYYT |
| | MSNNLVTLENGKQLTVKRQGLYYIYAQVTFCSNREASSQAPFIASLCLKSP |
| | GRFERILLRAANTHSSAKPCGQQSIHLGGVFELQPGASVFVNVTDPSQVSH |
| | GTGFTSFGLLKL |
| SEQ ID NO: 620 | GDQNPQIAAHVISEASSKTTSVLQWAEKGYYTMSNNLVTLENGKQLTVKR |
| CD40Lecd-Fc- | QGLYYIYAQVTFCSNREASSQAPFIASLCLKSPGRFERILLRAANTHSSAKPC |
| TGFbRecd | GQQSIHLGGVFELQPGASVFVNVTDPSQVSHGTGFTSFGLLKLASTKGPSVF |
| | PLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPSCPAPE |
| | FLGGPSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIAVEWESNG |
| | QPENNYKTTPPVLDSDGSFFLYSRLTVDKSRWQEGNVFSCSVMHEALHNH |
| | YTQKSLSLSLGKGGGGSGGGGSGGGGSTIPPHVQKSVNNDMIVTDNNGAV |
| | KFPQLCKFCDVRFSTCDNQKSCMSNCSITSICEKPQEVCVAVWRKNDENIT |
| | LETVCHDPKLPYHDFILEDAASPKCIMKEKKKPGETFFMCSCSSDECNDNII |
| | FSEEYNTSNPD |
| SEQ ID NO: 621 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| CD70ecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSQRFAQAQQQLPLESLGWDVAELQLNHTGPQQD |
| | PRLYWQGGPALGRSFLHGPELDKGQLRIHRDGIYMVHIQVTLAICSSTTASR |
| | HHPFILAVGICSPASRSISLLRLSFHQGCTIASQRLTPLARGDTLCTNLTGTL |
| | LPSRNTDETFFGVQWVRP |
| SEQ ID NO: 622 | QRFAQAQQQLPLESLGWDVAELQLNHTGPQQDPRLYWQGGPALGRSFLH |
| CD70ecd-Fc- | GPELDKGQLRIHRDGIYMVHIQVTLAICSSTTASRHHPTTLAVGICSPASRSI |
| TGFbRecd | SLLRLSFHQGCTIASQRLTPLARGDTLCTNLTGTLLPSRNTDETFFGVQWVR |
| | PASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKY |
| | GPPCPSCPAPEFLGGPSVFLFPPKPKDTLMISRTPEVTCVVVDVSQEDPEVQF |
| | NWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSCS |
| | VMHEALHNHYTQKSLSLSLGKGGGGSGGGGSGGGGSTIPPHVQKSVNND |
| | MIVTDNNGAVKFPQLCKFCDVRFSTCDNQKSCMSNCSITSICEKPQEVCVA |
| | VWRKNDENITLETVCHDPKLPYHDFILEDAASPKCIMKEKKKPGETFFMCS |
| | CSSDECNDNIIFSEEYNTSNPD |
| SEQ ID NO: 623 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| GITRLecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSQLETAKEPCMAKFGPLPSKWQMASSEPPCVNK |
| | VSDWKLEILQNGLYLIYGQVAPNANYNDVAPFEVRLYKNKDMIQTLTNKS |
| | KIQNVGGTYELHVGDTIDLIFNSEHQVLKNNTYWGIILLANPQFIS |
| SEQ ID NO: 624 | QLETAKEPCMAKFGPLPSKWQMASSEPPCVNKVSDWKLEILQNGLYLIYG |
| GITRLecd-Fc- | QVAPNANYNDVAPFEVRLYKNKDMIQTLTNKSKIQNVGGTYELHVGDTID |
| TGFbRecd | LIFNSEHQVLKNNTYWGIILLANPQFISASTKGPSVFPLAPCSRSTSESTAAL |
| | GCLVKDYFPEPVTVSWNSGALTSGVHTFPAVLQSSGLYSLSSVVTVPSSSL |
| | GTKTYTCNVDHKPSNTKVDKRVESKYGPPCPSCPAPEFLGGPSVFLFPPKPK |
| | DTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGKGG |
| | GGSGGGGSGGGGSTIPPHVQKSVNNDMIVTDNNGAVKFPQLCKFCDVRFS |
| | TCDNQKSCMSNCSITSICEKPQEVCVAVWRKNDENITLETVCHDPKLPYHD |
| | FILEDAASPKCIMKEKKKPGETFFMCSCSSDECNDNIIFSEEYNTSNPD |
| SEQ ID NO: 625 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| ICOSLecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSDTQEKEVRAMVGSDVELSCACPEGSRFDLNDV |
| | YVYWQTSESKTVVTYHIPQNSSLENVDSRYRNRALMSPAGMLRGDFSLRL |
| | FNVTPQDEQKFHCLVLSQSLGFQEVLSVEVTLHVAANFSVPVVSAPHSPSQ |
| | DELTFTCTSINGYPRPNVYWINKTDNSLLDQALQNDTVFLNMRGLYDVVS |
| | VLRIARTPSVNIGCCIENVLLQQNLTVGSQTGNDIGERDKITENPVSTGEKN |
| | AATWS |
| SEQ ID NO: 626 | DTQEKEVRAMVGSDVELSCACPEGSRFDLNDVYVYWQTSESKTVVTYHIP |
| ICOSLecd-Fc- | QNSSLENVDSRYRNRALMSPAGMLRGDFSLRLFNVTPQDEQKFHCLVLSQ |
| TGFbRecd | SLGFQEVLSVEVTLHVAANFSVPVVSAPHSPSQDELTFTCTSINGYPRPNVY |
| | WINKTDNSLLDQALQNDTVFLNMRGLYDVVSVLRIARTPSVNIGCCIENVL |
| | LQQNLTVGSQTGNDIGERDKITENPVSTGEKNAATWSASTKGPSVFPLAPC |
| | SRSTSESTAALGCLVKDYFPEPVTVSWNSGALTSGVHITPAVLQSSGLYSLS |
| | SVVTVPSSSLGTKTYTCNVDHKPSNTKVDKRVESKYGPPCPSCPAPEFLGG |
| | PSVFLFPPKPKDTLMISRTPEVTCVVVDVSQEDPEVQFNWYVDGVEVHNA |
| | KTKPREEQFNSTYRVVSVLTVLHQDWLNGKEYKCKVSNKGLPSSIEKTISK |
| | AKGQPREPQVYTLPPSQEEMTKNQVSLTCLVKGFYPSDIAVEWESNGQPEN |
| | NYKTTPPVLDSDGSFFLYSRLTVDKSRWQEGNVFSCSVMHEALHNHYTQK |
| | SLSLSLGKGGGGSGGGGSGGGGSTIPPHVQKSVNNDMIVTDNNGAVKFPQ |
| | LCKFCDVRFSTCDNQKSCMSNCSITSICEKPQEVCVAVWRKNDENITLETV |
| | CHDPKLPYHDFILEDAASPKCIMKEKKKPGETFFMCSCSSDECNDNIIFSEEY |
| | NTSNPD |
| SEQ ID NO: 627 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| LIGHTecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSDGPAGSWEQLIQERRSHEVNPAAHLTGANSSLT |
| | GSGGPLLWETQLGLAFLRGLSYHDGALVVTKAGYYYIYSKVQLGGVGCPL |
| | GLASTITHGLYKRTPRYPEELELLVSQQSPCGRATSSSRVWWDSSFLGGVV |
| | HLEAGEKVVVRVLDERLVRLRDGTRSYFGAFMV |
| SEQ ID NO: 628 | DGPAGSWEQLIQERRSHEVNPAAHLTGANSSLTGSGGPLLWETQLGLAFLR |
| LIGHTecd-Fc- | GLSYHDGALVVTKAGYYYIYSKVQLGGVGCPLGLASTITHGLYKRTPRYP |
| TGFbRecd | EELELLVSQQSPCGRATSSSRVWWDSSFLGGVVHLEAGEKVVVRVLDERL |
| | VRLRDGTRSYFGAFMVASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPSCPAPEFLGGPSVFLFPPKPKDTLMISRTPEVT |
| | CVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGKGGGGSGGGGSGGG |
| | GSTIPPHVQKSVNNDMIVTDNNGAVKFPQLCKFCDVRFSTCDNQKSCMSN |
| | CSITSICEKPQEVCVAVWRKNDENITLETVCHDPKLPYHDFILEDAASPKCI |
| | MKEKKKPGETFFMCSCSSDECNDNIIFSEEYNTSNPD |
| SEQ ID NO: 629 | TIPPHVQKSVNNDMIVTDNNGAVKFPQLCKFCDVRFSTCDNQKSCMSNCSI |
| TGFbRecd-Fc- | TSICEKPQEVCVAVWRKNDENITLETVCHDPKLPYHDFILEDAASPKCIMKE |
| OX40Lecd | KKKPGETFFMCSCSSDECNDNIIFSEEYNTSNPDASTKGPSVFPLAPCSRSTS |
| | ESTAALGCLVKDYFPEPVTVSWNSGALTSGVHTFPAVLQSSGLYSLSSVVT |
| | VPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTT |
| | PPVLDSDGSFFLYSRLTVDKSRWQEGNVFSCSVMHEALHNHYTQKSLSLSL |
| | GKGGGGSGGGGSGGGGSQVSHRYPRIQSIKVQFTEYKKEKGFILTSQKEDEI |
| | MKVQNNSVIINCDGFYLISLKGYFSQEVNISLHYQKDEEPLFQLKKVRSVNS |
| | LMVASLTYKDKVYLNVTTDNTSLDDFHVNGGELILIHQNPGEFCVL |
| SEQ ID NO: 630 | QVSHRYPRIQSIKVQFTEYKKEKGFILTSQKEDEIMKVQNNSVIINCDGFYLI |
| OX40Lecd-Fc- | SLKGYFSQEVNISLHYQKDEEPLFQLKKVRSVNSLMVASLTYKDKVYLNV |
| TGFbRecd | TTDNTSLDDFHVNGGELILIHQNPGEFCVLASTKGPSVFPLAPCSRSTSESTA |
| | ALGCLVKDYFPEPVTVSWNSGALTSGVHTFPAVLQSSGLYSLSSVVTVPSS |
| | SLGTKTYTCNVDHKPSNTKVDKRVESKYGPPCPSCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGKG |
| | GGGSGGGGSGGGGSTIPPHVQKSVNNDMIVTDNNGAVKFPQLCKFCDVRF |
| | STCDNQKSCMSNCSITSICEKPQEVCVAVWRKNDENITLETVCHDPKLPYH |
| | DFILEDAASPKCIMKEKKKPGETFFMCSCSSDECNDNIIFSEEYNTSNPD |
| SEQ ID NO: 631 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| 41BBLecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSACPWAVSGARASPGSAA |
| | SPRLREGPELSPDDPAGLLDLRQGMFAQLVAQNVLLIDGPLSWYSDPGLAG |
| | VSLTGGLSYKEDTKELVVAKAGVYYVFFQLELRRVVAGEGSGSVSLALHL |
| | QPLRSAAGAAALALTVDLPPASSEARNSAFGFQGRLLHLSAGQRLGVHLHT |
| | EARARHAWQLTQGATVLGLFRVTPEIPAGLPSPRSE |
| SEQ ID NO: 632 | ACPWAVSGARASPGSAASPRLREGPELSPDDPAGLLDLRQGMFAQLVAQN |
| 41BBLecd-Fc- | VLLIDGPLSWYSDPGLAGVSLTGGLSYKEDTKELVVAKAGVYYVFFQLEL |
| VEGFRecd | RRVVAGEGSGSVSLALHLQPLRSAAGAAALALTVDLPPASSEARNSAFGFQ |
| | GRLLHLSAGQRLGVHLHTEARARHAWQLTQGATVLGLFRVTPEIPAGLPSP |
| | RSEASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVES |
| | KYGPPCPSCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGKEYK |
| | CKVSNKGLPSSIEKTISKAKGQPREPQVYTLPPSQEEMTKNQVSLTCLVKGF |
| | YPSDIAVEWESNGQPENNYKTTPPVLDSDGSFFLYSRLTVDKSRWQEGNVF |
| | SCSVMHEALHNHYTQKSLSLSLGKGGGGSGGGGSGGGGSSDTGRPFVEMY |
| | SEIPEIIHMTEGRELVIPCRVTSPNITVTLKKFPLDTLIPDGKRIIWDSRKGFIIS |
| | NATYKEIGLLTCEATVNGHLYKTNYLTHRQTNTIIDVVLSPSHGIELSVGEK |
| | LVLNCTARTELNVGIDFNWEYPSSKHQHKKLVNRDLKTQSGSEMKKFLST |
| | LTIDGVTRSDQGLYTCAASSGLMTKKNSTFVRVHEK |
| SEQ ID NO: 633 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| CD30Lecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSQRTDSIPNSPDNVPLKGG |
| | NCSEDLLCILKRAPFKKSWAYLQVAKHLNKTKLSWNKDGILHGVRYQDG |
| | NLVIQFPGLYFIICQLQFLVQCPNNSVDLKLELLINKHIKKQALVTVCESGM |
| | QTKHVYQNLSQFLLDYLQVNTTISVNVDTFQYIDTSTFPLENVLSIFLYSNS |
| | D |
| SEQ ID NO: 634 | QRTDSIPNSPDNVPLKGGNCSEDLLCILKRAPFKKSWAYLQVAKHLNKTKL |
| CD30Lecd-Fc- | SWNKDGILHGVRYQDGNLVIQFPGLYFIICQLQFLVQCPNNSVDLKLELLIN |
| VEGFRecd | KHIKKQALVTVCESGMQTKHVYQNLSQFLLDYLQVNTTISVNVDTFQYIDT |
| | STFPLENVLSIFLYSNSDASTKGPSVFPLAPCSRSTSESTAALGCLVKDYFPE |
| | PVTVSWNSGALTSGVHTFPAVLQSSGLYSLSSVVTVPSSSLGTKTYTCNVD |
| | HKPSNTKVDKRVESKYGPPCPSCPAPEFLGGPSVFLFPPKPKDTLMISRTPE |
| | VTCVVVDVSQEDPEVQFNWYVDGVEVHNAKTKPREEQFNSTYRVVSVLT |
| | VLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSR |
| | LTVDKSRWQEGNVFSCSVMHEALHNHYTQKSLSLSLGKGGGGSGGGGSG |
| | GGGSSDTGRPFVEMYSEIPEIIHMTEGRELVIPCRVTSPNITVTLKKFPLDTLI |
| | PDGKRIIWDSRKGFIISNATYKEIGLLTCEATVNGHLYKTNYLTHRQTNTIID |
| | VVLSPSHGIELSVGEKLVLNCTARTELNVGIDFNWEYPSSKHQHKKLVNRD |
| | LKTQSGSEMKKFLSTLTIDGVTRSDQGLYTCAASSGLMTKKNSTFVRVHEK |
| SEQ ID NO: 635 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| CD40Lecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHITPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSGDQNPQIAAHVISEASSKT |
| | TSVLQWAEKGYYTMSNNLVTLENGKQLTVKRQGLYYIYAQVTFCSNREA |
| | SSQAPFIASLCLKSPGRFERILLRAANTHSSAKPCGQQSIHLGGVFELQPGAS |
| | VFVNVTDPSQVSHGTGFTSFGLLKL |
| SEQ ID NO: 636 | GDQNPQIAAHVISEASSKTTSVLQWAEKGYYTMSNNLVTLENGKQLTVKR |
| CD40Lecd-Fc- | QGLYYIYAQVTFCSNREASSQAPFIASLCLKSPGRFERILLRAANTHSSAKPC |
| VEGFRecd | GQQSIHLGGVFELQPGASVFVNVTDPSQVSHGTGFTSFGLLKLASTKGPSVF |
| | PLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPSCPAPE |
| | FLGGPSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIAVEWESNG |
| | QPENNYKTTPPVLDSDGSFFLYSRLTVDKSRWQEGNVFSCSVMHEALHNH |
| | YTQKSLSLSLGKGGGGSGGGGSGGGGSSDTGRPFVEMYSEIPEIIHMTEGRE |
| | LVIPCRVTSPNITVTLKKFPLDTLIPDGKRIIWDSRKGFIISNATYKEIGLLTCE |
| | ATVNGHLYKTNYLTHRQTNTIIDVVLSPSHGIELSVGEKLVLNCTARTELN |
| | VGIDFNWEYPSSKHQHKKLVNRDLKTQSGSEMKKFLSTLTIDGVTRSDQGL |
| | YTCAASSGLMTKKNSTFVRVHEK |
| SEQ ID NO: 637 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| CD70ecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHITPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSQRFAQAQQQLPLESLGW |
| | DVAELQLNHTGPQQDPRLYWQGGPALGRSFLHGPELDKGQLRIHRDGIYM |
| | VHIQVTLAICSSTTASRHHPTTLAVGICSPASRSISLLRLSFHQGCTIASQRLT |
| | PLARGDTLCTNLTGTLLPSRNTDEIFFGVQWVRP |
| SEQ ID NO: 638 | QRFAQAQQQLPLESLGWDVAELQLNHTGPQQDPRLYWQGGPALGRSFLH |
| CD70ecd-Fc - | GPELDKGQLRIHRDGIYMVHIQVTLAICSSTTASRHHPTTLAVGICSPASRSI |
| VEGFRecd | SLLRLSFHQGCTIASQRLTPLARGDTLCTNLTGTLLPSRNTDETFFGVQWVR |
| | PASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKY |
| | GPPCPSCPAPEFLGGPSVFLFPPKPKDTLMISRTPEVTCVVVDVSQEDPEVQF |
| | NWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSCS |
| | VMHEALHNHYTQKSLSLSLGKGGGGSGGGGSGGGGSSDTGRPFVEMYSEI |
| | PEIIHMTEGRELVIPCRVTSPNITVTLKKFPLDTLIPDGKRIIWDSRKGFIISNA |
| | TYKEIGLLTCEATVNGHLYKTNYLTHRQTNTIIDVVLSPSHGIELSVGEKLV |
| | LNCTARTELNVGIDFNWEYPSSKHQHKKLVNRDLKTQSGSEMKKFLSTLTI |
| | DGVTRSDQGLYTCAASSGLMTKKNSTFVRVHEK |
| SEQ ID NO: 639 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| GITRLecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHITPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSQLETAKEPCMAKFGPLPS |
| | KWQMASSEPPCVNKVSDWKLEILQNGLYLIYGQVAPNANYNDVAPFEVRL |
| | YKNKDMIQTLTNKSKIQNVGGTYELHVGDTIDLIFNSEHQVLKNNTYWGII |
| | LLANPQFIS |
| SEQ ID NO: 640 | QLETAKEPCMAKFGPLPSKWQMASSEPPCVNKVSDWKLEILQNGLYLIYG |
| GITRLecd-Fc- | QVAPNANYNDVAPFEVRLYKNKDMIQTLTNKSKIQNVGGTYELHVGDTID |
| VEGFRecd | LIFNSEHQVLKNNTYWGIILLANPQFISASTKGPSVFPLAPCSRSTSESTAAL |
| | GCLVKDYFPEPVTVSWNSGALTSGVHTFPAVLQSSGLYSLSSVVTVPSSSL |
| | GTKTYTCNVDHKPSNTKVDKRVESKYGPPCPSCPAPEFLGGPSVFLFPPKPK |
| | DTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGKGG |
| | GGSGGGGSGGGGSSDTGRPFVEMYSEIPEIIHMTEGRELVIPCRVTSPNITVT |
| | LKKFPLDTLIPDGKRIIWDSRKGFIISNATYKEIGLLTCEATVNGHLYKTNYL |
| | THRQTNTIIDVVLSPSHGIELSVGEKLVLNCTARTELNVGIDFNWEYPSSKH |
| | QHKKLVNRDLKTQSGSEMKKFLSTLTIDGVTRSDQGLYTCAASSGLMTKK |
| | NSTFVRVHEK |
| SEQ ID NO: 641 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| ICOSLecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSDTQEKEVRAMVGSDVEL |
| | SCACPEGSRFDLNDVYVYWQTSESKTVVTYHIPQNSSLENVDSRYRNRAL |
| | MSPAGMLRGDFSLRLFNVTPQDEQKFHCLVLSQSLGFQEVLSVEVTLHVA |
| | ANFSVPVVSAPHSPSQDELTFTCTSINGYPRPNVYWINKTDNSLLDQALQN |
| | DTVFLNMRGLYDVVSVLRIARTPSVNIGCCIENVLLQQNLTVGSQTGNDIG |
| | ERDKITENPVSTGEKNAATWS |
| SEQ ID NO: 642 | DTQEKEVRAMVGSDVELSCACPEGSRFDLNDVYVYWQTSESKTVVTYHIP |
| ICOSLecd-Fc- | QNSSLENVDSRYRNRALMSPAGMLRGDFSLRLFNVTPQDEQKFHCLVLSQ |
| VEGFRecd | SLGFQEVLSVEVTLHVAANFSVPVVSAPHSPSQDELTFTCTSINGYPRPNVY |
| | WINKTDNSLLDQALQNDTVFLNMRGLYDVVSVLRIARTPSVNIGCCIENVL |
| | LQQNLTVGSQTGNDIGERDKITENPVSTGEKNAATWSASTKGPSVFPLAPC |
| | SRSTSESTAALGCLVKDYFPEPVTVSWNSGALTSGVHTFPAVLQSSGLYSLS |
| | SVVTVPSSSLGTKTYTCNVDHKPSNTKVDKRVESKYGPPCPSCPAPEFLGG |
| | PSVFLFPPKPKDTLMISRTPEVTCVVVDVSQEDPEVQFNWYVDGVEVHNA |
| | KTKPREEQFNSTYRVVSVLTVLHQDWLNGKEYKCKVSNKGLPSSIEKTISK |
| | AKGQPREPQVYTLPPSQEEMTKNQVSLTCLVKGFYPSDIAVEWESNGQPEN |
| | NYKTTPPVLDSDGSFFLYSRLTVDKSRWQEGNVFSCSVMHEALHNHYTQK |
| | SLSLSLGKGGGGSGGGGSGGGGSSDTGRPFVEMYSEIPEIIHMTEGRELVIP |
| | CRVTSPNITVTLKKFPLDTLIPDGKRIIWDSRKGFIISNATYKEIGLLTCEATV |
| | NGHLYKTNYLTHRQTNTIIDVVLSPSHGIELSVGEKLVLNCTARTELNVGID |
| | FNWEYPSSKHQHKKLVNRDLKTQSGSEMKKFLSTLTIDGVTRSDQGLYTC |
| | AASSGLMTKKNSTFVRVHEK |
| SEQ ID NO: 643 | SDTGRPFVEMYSEIPEIIHMTEGRELVIPCRVTSPNITVTLKKFPLDTLIPDGK |
| VEGFRecd-Fc- | RIIWDSRKGFIISNATYKEIGLLTCEATVNGHLYKTNYLTHRQTNTIIDVVLS |
| LIGHTecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSDGPAGSWEQLIQERRSHE |
| | VNPAAHLTGANSSLTGSGGPLLWETQLGLAFLRGLSYHDGALVVTKAGYY |
| | YIYSKVQLGGVGCPLGLASTITHGLYKRTPRYPEELELLVSQQSPCGRATSS |
| | SRVWWDSSFLGGVVHLEAGEKVVVRVLDERLVRLRDGTRSYFGAFMV |
| SEQ ID NO: 644 | DGPAGSWEQLIQERRSHEVNPAAHLTGANSSLTGSGGPLLWETQLGLAFLR |
| LIGHTecd-Fc- | GLSYHDGALVVTKAGYYYIYSKVQLGGVGCPLGLASTITHGLYKRTPRYP |
| VEGFRecd | EELELLVSQQSPCGRATSSSRVWWDSSFLGGVVHLEAGEKVVVRVLDERL |
| | VRLRDGTRSYFGAFMVASTKGPSVFPLAPCSRSTSESTAALGCLVKDYFPEP |
| | VTVSWNSGALTSGVHTFPAVLQSSGLYSLSSVVTVPSSSLGTKTYTCNVDH |
| | KPSNTKVDKRVESKYGPPCPSCPAPEFLGGPSVFLFPPKPKDTLMISRTPEVT |
| | CVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLT |
| | VDKSRWQEGNVFSCSVMHEALHNHYTQKSLSLSLGKGGGGSGGGGSGGG |
| | GSSDTGRPFVEMYSEIPEIIHMTEGRELVIPCRVTSPNITVTLKKFPLDTLIPD |
| | GKRIIWDSRKGFIISNATYKEIGLLTCEATVNGHLYKTNYLTHRQTNTIIDVV |
| | LSPSHGIELSVGEKLVLNCTARTELNVGIDFNWEYPSSKHQHKKLVNRDLK |
| | TQSGSEMKKFLSTLTIDGVTRSDQGLYTCAASSGLMTKKNSTFVRVHEK |
| SEQ ID NO: 645 | SDTGRPFVEMYSEIPEIIHMTEGRELVIPCRVTSPNITVTLKKFPLDTLIPDGK |
| VEGFRecd-Fc - | RIIWDSRKGFIISNATYKEIGLLTCEATVNGHLYKTNYLTHRQTNTIIDVVLS |
| OX40Lecd | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKASTK |
| | GPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPS |
| | CPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGL |
| | PSSIEKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCSVMHE |
| | ALHNHYTQKSLSLSLGKGGGGSGGGGSGGGGSQVSHRYPRIQSIKVQFTEY |
| | KKEKGFILTSQKEDEIMKVQNNSVIINCDGFYLISLKGYFSQEVNISLHYQK |
| | DEEPLFQLKKVRSVNSLMVASLTYKDKVYLNVTTDNTSLDDFHVNGGELI |
| | LIHQNPGEFCVL |
| SEQ ID NO: 646 | QVSHRYPRIQSIKVQFTEYKKEKGFILTSQKEDEIMKVQNNSVIINCDGFYLI |
| OX40Lecd-Fc- | SLKGYFSQEVNISLHYQKDEEPLFQLKKVRSVNSLMVASLTYKDKVYLNV |
| VEGFRecd | TTDNTSLDDFHVNGGELILIHQNPGEFCVLASTKGPSVFPLAPCSRSTSESTA |
| | ALGCLVKDYFPEPVTVSWNSGALTSGVHTFPAVLQSSGLYSLSSVVTVPSS |
| | SLGTKTYTCNVDHKPSNTKVDKRVESKYGPPCPSCPAPEFLGGPSVFLFPPK |
| | PKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVL |
| | DSDGSFFLYSRLTVDKSRWQEGNVFSCSVMHEALHNHYTQKSLSLSLGKG |
| | GGGSGGGGSGGGGSSDTGRPFVEMYSEIPEIIHMTEGRELVIPCRVTSPNITV |
| | TLKKFPLDTLIPDGKRIIWDSRKGFIISNATYKEIGLLTCEATVNGHLYKTNY |
| | LTHRQTNTIIDVVLSPSHGIELSVGEKLVLNCTARTELNVGIDFNWEYPSSK |
| | HQHKKLVNRDLKTQSGSEMKKFLSTLTIDGVTRSDQGLYTCAASSGLMTK |
| | KNSTFVRVHEK |
| SEQ ID NO: 647 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to 41BBL | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSACPWAVSGARASPGSAASPRLREGPELSPDDPAGLLDLRQG |
| | MFAQLVAQNVLLIDGPLSWYSDPGLAGVSLTGGLSYKEDTKELVVAKAGV |
| | YYVFFQLELRRVVAGEGSGSVSLALHLQPLRSAAGAAALALTVDLPPASSE |
| | ARNSAFGFQGRLLHLSAGQRLGVHLHTEARARHAWQLTQGATVLGLFRV |
| | TPEIPAGLPSPRSE |
| SEQ ID NO: 648 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to CD30L | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSQRTDSIPNSPDNVPLKGGNCSEDLLCILKRAPFKKSWAYLQV |
| | AKHLNKTKLSWNKDGILHGVRYQDGNLVIQFPGLYFIICQLQFLVQCPNNS |
| | VDLKLELLINKHIKKQALVTVCESGMQTKHVYQNLSQFLLDYLQVNTTISV |
| | NVDTFQYIDTSTFPLENVLSIFLYSNSD |
| SEQ ID NO: 649 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to CD40L | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSGDQNPQIAAHVISEASSKTTSVLQWAEKGYYTMSNNLVTLE |
| | NGKQLTVKRQGLYYIYAQVTFCSNREASSQAPFIASLCLKSPGRFERILLRA |
| | ANTHSSAKPCGQQSIHLGGVFELQPGASVFVNVTDPSQVSHGTGFTSFGLL |
| | KL |
| SEQ ID NO: 650 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to CD70 | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSQRFAQAQQQLPLESLGWDVAELQLNHTGPQQDPRLYWQGG |
| | PALGRSFLHGPELDKGQLRIHRDGIYMVHIQVTLAICSSTTASRHHPTTLAV |
| | GICSPASRSISLLRLSFHQGCTIASQRLTPLARGDTLCTNLTGTLLPSRNTDET |
| | FFGVQWVRP |
| SEQ ID NO: 651 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to GITRL | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSQLETAKEPCMAKFGPLPSKWQMASSEPPCVNKVSDWKLEIL |
| | QNGLYLIYGQVAPNANYNDVAPFEVRLYKNKDMIQTLTNKSKIQNVGGTY |
| | ELHVGDTIDLIFNSEHQVLKNNTYWGIILLANPQFIS |
| SEQ ID NO: 652 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to ICOSL | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSDTQEKEVRAMVGSDVELSCACPEGSRFDLNDVYVYWQTSE |
| | SKTVVTYHIPQNSSLENVDSRYRNRALMSPAGMLRGDFSLRLFNVTPQDEQ |
| | KFHCLVLSQSLGFQEVLSVEVTLHVAANFSVPVVSAPHSPSQDELTFTCTSI |
| | NGYPRPNVYWINKTDNSLLDQALQNDTVFLNMRGLYDVVSVLRIARTPSV |
| | NIGCCIENVLLQQNLTVGSQTGNDIGERDKITENPVSTGEKNAATWS |
| SEQ ID NO: 653 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to IL12 | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSIWELKKDVYVVELDWYPDAPGEMVVLTCDTPEEDGITWTL |
| | DQSSEVLGSGKTLTIQVKEFGDAGQYTCHKGGEVLSHSLLLLHKKEDGIWS |
| | TDILKDQKEPKNKTFLRCEAKNYSGRFTCWWLTTISTDLTFSVKSSRGSSDP |
| | QGVTCGAATLSAERVRGDNKEYEYSVECQEDSACPAAEESLPIEVMVDAV |
| | HKLKYENYTSSFFIRDIIKPDPPKNLQLKPLKNSRQVEVSWEYPDTWSTPHS |
| | YFSLTFCVQVQGKSKREKKDRVFTDKTSATVICRKNASISVRAQDRYYSSS |
| | WSEWASVPCSGGGGSGGGGSGGGGSRNLPVATPDPGMFPCLHHSQNLLR |
| | AVSNMLQKARQTLEFYPCTSEEIDHEDITKDKTSTVEACLPLELTKNESCLN |
| | SRETSFITNGSCLASRKTSFMMALCLSSIYEDLKMYQVEFKTMNAKLLMDP |
| | KRQIFLDQNMLAVIDELMQALNFNSETVPQKSSLEEPDFYKTKIKLCILLHA |
| | FRIRAVTIDRVMSYLNAS |
| SEQ ID NO: 654 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to IL15 | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSNWVNVISDLKKIEDLIQSMHIDATLYTESDVHPSCKVTAMK |
| | CFLLELQVISLESGDASIHDTVENLIILANNSLSSNGNVTESGCKECEELEEK |
| | NIKEFLQSFVHIVQMFINTS |
| SEQ ID NO: 655 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to LIGHT | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSDGPAGSWEQLIQERRSHEVNPAAHLTGANSSLTGSGGPLLW |
| | ETQLGLAFLRGLSYHDGALVVTKAGYYYIYSKVQLGGVGCPLGLASTITH |
| | GLYKRTPRYPEELELLVSQQSPCGRATSSSRVWWDSSFLGGVVHLEAGEK |
| | VVVRVLDERLVRLRDGTRSYFGAFMV |
| SEQ ID NO: 656 | EVQLVESGGGLVQPGGSLRLSCAASGFTFSDSWIHWVRQAPGKGLEWVA |
| anti-PDL1 HC | WISPYGGSTYYADSVKGRFTISADTSKNTAYLQMNSLRAEDTAVYYCARR |
| fused to OX40L | HWPGGFDYWGQGTLVTVSAASTKGPSVFPLAPSSKSTSGGTAALGCLVKD |
| ECD | YFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYASTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGKGGGGSG |
| | GGGSGGGGSQVSHRYPRIQSIKVQFTEYKKEKGFILTSQKEDEIMKVQNNS |
| | VIINCDGFYLISLKGYFSQEVNISLHYQKDEEPLFQLKKVRSVNSLMVASLT |
| | YKDKVYLNVTTDNTSLDDFHVNGGELILIHQNPGEFCVL |
| SEQ ID NO: 657 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to 41BBL | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSACPWAVSGARASPGSAASPRLREGPE |
| | LSPDDPAGLLDLRQGMFAQLVAQNVLLIDGPLSWYSDPGLAGVSLTGGLS |
| | YKEDTKELVVAKAGVYYVFFQLELRRVVAGEGSGSVSLALHLQPLRSAAG |
| | AAALALTVDLPPASSEARNSAFGFQGRLLHLSAGQRLGVHLHTEARARHA |
| | WQLTQGATVLGLFRVTPEIPAGLPSPRSE |
| SEQ ID NO: 658 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to CD30L | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQRTDSIPNSPDNVPLKGGNCSEDLLCI |
| | LKRAPFKKSWAYLQVAKHLNKTKLSWNKDGILHGVRYQDGNLVIQFPGL |
| | YFIICQLQFLVQCPNNSVDLKLELLINKHIKKQALVTVCESGMQTKHVYQN |
| | LSQFLLDYLQVNTTISVNVDTFQYIDTSTFPLENVLSIFLYSNSD |
| SEQ ID NO: 659 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to CD40L | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSGDQNPQIAAHVISEASSKTTSVLQWA |
| | EKGYYTMSNNLVTLENGKQLTVKRQGLYYIYAQVTFCSNREASSQAPFIAS |
| | LCLKSPGRFERILLRAANTHSSAKPCGQQSIHLGGVFELQPGASVFVNVTDP |
| | SQVSHGTGFTSFGLLKL |
| SEQ ID NO: 660 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to CD70 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQRFAQAQQQLPLESLGWDVAELQLN |
| | HTGPQQDPRLYWQGGPALGRSFLHGPELDKGQLRIHRDGIYMVHIQVTLAI |
| | CSSTTASRHHPTTLAVGICSPASRSISLLRLSFHQGCTIASQRLTPLARGDTLC |
| | TNLTGTLLPSRNTDETFFGVQWVRP |
| SEQ ID NO: 661 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to GITRL | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQLETAKEPCMAKFGPLPSKWQMASSE |
| | PPCVNKVSDWKLEILQNGLYLIYGQVAPNANYNDVAPFEVRLYKNKDMIQ |
| | TLTNKSKIQNVGGTYELHVGDTIDLIFNSEHQVLKNNTYWGIILLANPQFIS |
| SEQ ID NO: 662 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to ICOSL | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSDTQEKEVRAMVGSDVELSCACPEGSR |
| | FDLNDVYVYWQTSESKTVVTYHIPQNSSLENVDSRYRNRALMSPAGMLRG |
| | DFSLRLFNVTPQDEQKFHCLVLSQSLGFQEVLSVEVTLHVAANFSVPVVSA |
| | PHSPSQDELTFTCTSINGYPRPNVYWINKTDNSLLDQALQNDTVFLNMRGL |
| | YDVVSVLRIARTPSVNIGCCIENVLLQQNLTVGSQTGNDIGERDKITENPVS |
| | TGEKNAATWS |
| SEQ ID NO: 663 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to IL12 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSIWELKKDVYVVELDWYPDAPGEMVV |
| | LTCDTPEEDGITWTLDQSSEVLGSGKTLTIQVKEFGDAGQYTCHKGGEVLS |
| | HSLLLLHKKEDGIWSTDILKDQKEPKNKTFLRCEAKNYSGRFTCWWLTTIS |
| | TDLTFSVKSSRGSSDPQGVTCGAATLSAERVRGDNKEYEYSVECQEDSACP |
| | AAEESLPIEVMVDAVHKLKYENYTSSFFIRDIIKPDPPKNLQLKPLKNSRQV |
| | EVSWEYPDTWSTPHSYFSLTFCVQVQGKSKREKKDRVFTDKTSATVICRK |
| | NASISVRAQDRYYSSSWSEWASVPCSGGGGSGGGGSGGGGSRNLPVATPD |
| | PGMFPCLHHSQNLLRAVSNMLQKARQTLEFYPCTSEEIDHEDITKDKTSTV |
| | EACLPLELTKNESCLNSRETSFITNGSCLASRKTSFMMALCLSSIYEDLKMY |
| | QVEFKTMNAKLLMDPKRQIFLDQNMLAV1DELMQALNFNSETVPQKSSLE |
| | EPDFYKTKIKLCILLHAFRIRAVTIDRVMSYLNAS |
| SEQ ID NO: 664 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to IL15 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSNWVNVISDLKKIEDLIQSMHIDATLYT |
| | ESDVHPSCKVTAMKCFLLELQVISLESGDASIHDTVENLIILANNSLSSNGN |
| | VTESGCKECEELEEKNIKEFLQSFVHIVQMFINTS |
| SEQ ID NO: 665 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to LIGHT | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSDGPAGSWEQLIQERRSHEVNPAAHLT |
| | GANSSLTGSGGPLLWETQLGLAFLRGLSYHDGALVVTKAGYYYIYSKVQL |
| | GGVGCPLGLASTITHGLYKRTPRYPEELELLVSQQSPCGRATSSSRVWWDS |
| | SFLGGVVHLEAGEKVVVRVLDERLVRLRDGTRSYFGAFMV |
| SEQ ID NO: 666 | DIQMTQSPSSLSASVGDRVTITCRASQDVSTAVAWYQQKPGKAPKLLIYSA |
| anti-PDL1 LC | SFLYSGVPSRFSGSGSGTDFTLTISSLQPEDFATYYCQQYLYHPATFGQGTK |
| fused to OX40L | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQVSHRYPRIQSIKVQFTEYKKEKGFIL |
| | TSQKEDEIMKVQNNSVIINCDGFYLISLKGYFSQEVNISLHYQKDEEPLFQL |
| | KKVRSVNSLMVASLTYKDKVYLNVTTDNTSLDDFHVNGGELILIHQNPGE |
| | FCVL |
| SEQ ID NO: 667 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to 41BBL | DYEFAYWGQGTLVTVSAASTKGPSVFPLAPSSKSTSGGTAALGCLVKDYFP |
| ECD | EPVTVSWNSGALTSGVHTFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSACPWAVSGARASPGSAASPRLREGPELSPDDPAGLLDLRQGMF |
| | AQLVAQNVLLIDGPLSWYSDPGLAGVSLTGGLSYKEDTKELVVAKAGVYY |
| | VFFQLELRRVVAGEGSGSVSLALHLQPLRSAAGAAALALTVDLPPASSEAR |
| | NSAFGFQGRLLHLSAGQRLGVHLHTEARARHAWQLTQGATVLGLFRVTPE |
| | IPAGLPSPRSE |
| SEQ ID NO: 668 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to CD30L | DYEFAYWGQGTLVTVSAASTKGPSVFPLAPSSKSTSGGTAALGCLVKDYFP |
| ECD | EPVTVSWNSGALTSGVHTFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSQRTDSIPNSPDNVPLKGGNCSEDLLCILKRAPFKKSWAYLQVAK |
| | HLNKTKLSWNKDGILHGVRYQDGNLVIQFPGLYFIICQLQFLVQCPNNSVD |
| | LKLELLINKHIKKQALVTVCESGMQTKHVYQNLSQFLLDYLQVNTTISVNV |
| | DTFQYIDTSTFPLENVLSIFLYSNSD |
| SEQ ID NO: 669 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to CD40L | DYEFAYWGQGTLVTVSAASTKGPSVFPLAPSSKSTSGGTAALGCLVKDYFP |
| ECD | EPVTVSWNSGALTSGVHTFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSGDQNPQIAAHVISEASSKTTSVLQWAEKGYYTMSNNLVTLENG |
| | KQLTVKRQGLYYIYAQVTFCSNREASSQAPFIASLCLKSPGRFERILLRAAN |
| | THSSAKPCGQQSIHLGGVFELQPGASVFVNVTDPSQVSHGTGFTSFGLLKL |
| SEQ ID NO: 670 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to CD70 | DYEFAYWGQGTLVTVSAASTKGPSVFPLAPSSKSTSGGTAALGCLVKDYFP |
| ECD | EPVTVSWNSGALTSGVHTFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSQRFAQAQQQLPLESLGWDVAELQLNHTGPQQDPRLYWQGGPA |
| | LGRSFLHGPELDKGQLRIHRDGIYMVHIQVTLAICSSTTASRHHPTTLAVGI |
| | CSPASRSISLLRLSFHQGCTIASQRLTPLARGDTLCTNLTGTLLPSRNTDETFF |
| | GVQWVRP |
| SEQ ID NO: 671 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to GITRL | DYEFAYWGQGTLVTVSAASTKGPSVFPLAPSSKSTSGGTAALGCLVKDYFP |
| ECD | EPVTVSWNSGALTSGVHTFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSQLETAKEPCMAKFGPLPSKWQMASSEPPCVNKVSDWKLEILQN |
| | GLYLIYGQVAPNANYNDVAPFEVRLYKNKDMIQTLTNKSKIQNVGGTYEL |
| | HVGDTIDLIFNSEHQVLKNNTYWGIILLANPQFIS |
| SEQ ID NO: 672 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to ICOSL | DYEFAYWGQGTLVTVSAASTKGPSVFPLAPSSKSTSGGTAALGCLVKDYFP |
| ECD | EPVTVSWNSGALTSGVHTFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSDTQEKEVRAMVGSDVELSCACPEGSRFDLNDVYVYWQTSESK |
| | TVVTYHIPQNSSLENVDSRYRNRALMSPAGMLRGDFSLRLFNVTPQDEQKF |
| | HCLVLSQSLGFQEVLSVEVTLHVAANFSVPVVSAPHSPSQDELTFTCTSING |
| | YPRPNVYWINKTDNSLLDQALQNDTVFLNMRGLYDVVSVLRIARTPSVNI |
| | GCCIENVLLQQNLTVGSQTGNDIGERDKITENPVSTGEKNAATWS |
| SEQ ID NO: 673 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to IL12 | DYEFAYWGQGTLVTVSAASTKGPSVFPLAPSSKSTSGGTAALGCLVKDYFP |
| ECD | EPVTVSWNSGALTSGVHIFYAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSIWELKKDVYVVELDWYPDAPGEMVVLTCDTPEEDGITWTLDQ |
| | SSEVLGSGKTLTIQVKEFGDAGQYTCHKGGEVLSHSLLLLHKKEDGIWSTD |
| | ILKDQKEPKNKTFLRCEAKNYSGRFTCWWLTTISTDLTFSVKSSRGSSDPQG |
| | VTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYF |
| | SLTFCVQVQGKSKREKKDRVFTDKTSATVICRKNASISVRAQDRYYSSSWS |
| | EWASVPCSGGGGSGGGGSGGGGSRNLPVATPDPGMFPCLHHSQNLLRAVS |
| | NMLQKARQTLEFYPCTSEEIDHEDITKDKTSTVEACLPLELTKNESCLNSRE |
| | TSFITNGSCLASRKTSFMNIALCLSSIYEDLKMYQVEFKTMNAKLLMDPKR |
| | QIFLDQNMLAVIDELMQALNFNSETVPQKSSLEEPDFYKTKIKLCILLHAFRI |
| | RAVTIDRVMSYLNAS |
| SEQ ID NO: 674 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to IL15 | DYEFAYWGQGTLVTVSAASTKGPSVFPLAPSSKSTSGGTAALGCLVKDYFP |
| ECD | EPVTVSWNSGALTSGVHTFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSNWVNVISDLKKIEDLIQSMHIDATLYTESDVHPSCKVTAMKCFL |
| | LELQVISLESGDASIHDTVENLIILANNSLSSNGNVTESGCKECEELEEKNIK |
| | EFLQSFVHIVQMFINTS |
| SEQ ID NO: 675 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to LIGHT | DYEFAYWGQGTLVTVSAASTKGPSVFPLAPSSKSTSGGTAALGCLVKDYFP |
| ECD | EPVTVSWNSGALTSGVHTFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSDGPAGSWEQLIQERRSHEVNPAAHLTGANSSLTGSGGPLLWET |
| | QLGLAFLRGLSYHDGALVVTKAGYYYIYSKVQLGGVGCPLGLASTITHGL |
| | YKRTPRYPEELELLVSQQSPCGRATSSSRVWWDSSFLGGVVHLEAGEKVV |
| | VRVLDERLVRLRDGTRSYFGAFMV |
| SEQ ID NO: 676 | QVQLKQSGPGLVQPSQSLSITCTVSGFSLTNYGVHWVRQSPGKGLEWLGVI |
| anti-EGFR HC | WSGGNTDYNTPFTSRLSINKDNSKSQVFFKMNSLQSNDTAIYYCARALTYY |
| fused to OX40L | DYEFAYWGQGTLVTVSAASTKGPSVFPLAPSSKSTSGGTAALGCLVKDYFP |
| ECD | EPVTVSWNSGALTSGVHTFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGGGSGGG |
| | GSGGGGSQVSHRYPRIQSIKVQFTEYKKEKGFILTSQKEDEIMKVQNNSVII |
| | NCDGFYLISLKGYFSQEVNISLHYQKDEEPLFQLKKVRSVNSLMVASLTYK |
| | DKVYLNVTTDNTSLDDFHVNGGELILIHQNPGEFCVL |
| SEQ ID NO: 677 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to 41BBL | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSACPWAVSGARASPGSAASPRLREGPELSPD |
| | DPAGLLDLRQGMFAQLVAQNVLLIDGPLSWYSDPGLAGVSLTGGLSYKED |
| | TKELVVAKAGVYYVFFQLELRRVVAGEGSGSVSLALHLQPLRSAAGAAAL |
| | ALTVDLPPASSEARNSAFGFQGRLLHLSAGQRLGVHLHTEARARHAWQLT |
| | QGATVLGLFRVTPEIPAGLPSPRSE |
| SEQ ID NO: 678 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to CD30L | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSQRTDSIPNSPDNVPLKGGNCSEDLLCILKRA |
| | PFKKSWAYLQVAKHLNKTKLSWNKDGILHGVRYQDGNLVIQFPGLYFIIC |
| | QLQFLVQCPNNSVDLKLELLINKHIKKQALVTVCESGMQTKHVYQNLSQF |
| | LLDYLQVNTTISVNVDTFQYIDTSTFPLENVLSIFLYSNSD |
| SEQ ID NO: 679 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to CD40L | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSGDQNPQIAAHVISEASSKTTSVLQWAEKGY |
| | YTMSNNLVTLENGKQLTVKRQGLYYIYAQVTFCSNREASSQAPFIASLCLK |
| | SPGRFERILLRAANTHSSAKPCGQQSIHLGGVFELQPGASVFVNVTDPSQVS |
| | HGTGFTSFGLLKL |
| SEQ ID NO: 680 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to CD70 | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSQRFAQAQQQLPLESLGWDVAELQLNHTGP |
| | QQDPRLYWQGGPALGRSFLHGPELDKGQLRIHRDGIYMVHIQVTLAICSST |
| | TASRHHPTTLAVGICSPASRSISLLRLSFHQGCTIASQRLTPLARGDTLCTNL |
| | TGTLLPSRNTDETFFGVQWVRP |
| SEQ ID NO: 681 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to GITRL | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSQLETAKEPCMAKFGPLPSKWQMASSEPPC |
| | VNKVSDWKLEILQNGLYLIYGQVAPNANYNDVAPFEVRLYKNKDMIQTLT |
| | NKSKIQNVGGTYELHVGDTIDLIFNSEHQVLKNNTYWGIILLANPQFIS |
| SEQ ID NO: 682 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to ICOSL | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSDTQEKEVRAMVGSDVELSCACPEGSRFDL |
| | NDVYVYWQTSESKTVVTYHIPQNSSLENVDSRYRNRALMSPAGMLRGDFS |
| | LRLFNVTPQDEQKFHCLVLSQSLGFQEVLSVEVTLHVAANFSVPVVSAPHS |
| | PSQDELTFTCTSINGYPRPNVYWINKTDNSLLDQALQNDTVFLNMRGLYDV |
| | VSVLRIARTPSVNIGCCIENVLLQQNLTVGSQTGNDIGERDKITENPVSTGEK |
| | NAATWS |
| SEQ ID NO: 683 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to IL12 | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSIWELKKDVYVVELDWYPDAPGEMVVLTC |
| | DTPEEDGITWTLDQSSEVLGSGKTLTIQVKEFGDAGQYTCHKGGEVLSHSL |
| | LLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAE |
| | ESLPIEVMVDAVHKLKYENYTSSFFIRDIIKPDPPKNLQLKPLKNSRQVEVS |
| | WEYPDTWSTPHSYFSLTFCVQVQGKSKREKKDRVFTDKTSATVICRKNASI |
| | SVRAQDRYYSSSWSEWASVPCSGGGGSGGGGSGGGGSRNLPVATPDPGM |
| | FPCLHHSQNLLRAVSNMLQKARQTLEFYPCTSEEIDHEDITKDKTSTVEACL |
| | PLELTKNESCLNSRETSFITNGSCLASRKTSFMNIALCLSSIYEDLKMYQVEF |
| | KTMNAKLLMDPKRQIFLDQNMLAVIDELMQALNFNSETVPQKSSLEEPDF |
| | YKTKIKLCILLHAFRIRAVTIDRVMSYLNAS |
| SEQ ID NO: 684 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to IL15 | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSNWVNVISDLKKIEDLIQSMHIDATLYTESD |
| | VHPSCKVTAMKCFLLELQVISLESGDASIHDTVENLIILANNSLSSNGNVTES |
| | GCKECEELEEKNIKEFLQSFVHIVQMFINTS |
| SEQ ID NO: 685 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to LIGHT | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSDGPAGSWEQLIQERRSHEVNPAAHLTGANS |
| | SLTGSGGPLLWETQLGLAFLRGLSYHDGALVVTKAGYYYIYSKVQLGGVG |
| | CPLGLASTITHGLYKRTPRYPEELELLVSQQSPCGRATSSSRVWWDSSFLGG |
| | VVHLEAGEKVVVRVLDERLVRLRDGTRSYFGAFMV |
| SEQ ID NO: 686 | DILLTQSPVILSVSPGERVSFSCRASQSIGTNIHWYQQRTNGSPRLLIKYASES |
| anti-EGFR LC | ISGIPSRFSGSGSGTDFTLSINSVESEDIADYYCQQNNNWPTTFGAGTKLELK |
| fused to OX40L | RTVAAPSVFIFPPSDEQLKSGTASVVCLLNNFYPREAKVQWKVDNALQSGN |
| ECD | SQESVTEQDSKDSTYSLSSTLTLSKADYEKHKVYACEVTHQGLSSPVTKSF |
| | NRGECGGGGSGGGGSGGGGSQVSHRYPRIQSIKVQFTEYKKEKGFILTSQK |
| | EDEIMKVQNNSVIINCDGFYLISLKGYFSQEVNISLHYQKDEEPLFQLKKVR |
| | SVNSLMVASLTYKDKVYLNVTTDNTSLDDFHVNGGELILIHQNPGEFCVL |
| SEQ ID NO: 687 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to 41BBL | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | STKNQVSLTCLVKGFYPSDIAVEWESNGQPENNYKTTPPMLDDGSFFLYSK |
| | LTVDKSRWQQGNVFSCSVMHEALHNHYTQKSLSLSPGKGGGGSGGGGSG |
| | GGGSACPWAVSGARASPGSAASPRLREGPELSPDDPAGLLDLRQGMFAQL |
| | VAQNVLLIDGPLSWYSDPGLAGVSLTGGLSYKEDTKELVVAKAGVYYVFF |
| | QLELRRVVAGEGSGSVSLALHLQPLRSAAGAAALALTVDLPPASSEARNSA |
| | FGFQGRLLHLSAGQRLGVHLHTEARARHAWQLTQGATVLGLFRVTPEIPA |
| | GLPSPRSE |
| SEQ ID NO: 688 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to CD30L | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSQRTDSIPNSPDNVPLKGGNCSEDLLCILKRAPFKKSWAYLQVAKHLN |
| | KTKLSWNKDGILHGVRYQDGNLVIQFPGLYFIICQLQFLVQCPNNSVDLKL |
| | ELLINKHIKKQALVTVCESGMQTKHVYQNLSQFLLDYLQVNTTISVNVDTF |
| | QYIDTSTFPLENVLSIFLYSNSD |
| SEQ ID NO: 689 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to CD40L | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSGDQNPQIAAHVISEASSKITSVLQWAEKGYYTMSNNLVTLENGKQL |
| | TVKRQGLYYIYAQVTFCSNREASSQAPFIASLCLKSPGRFERILLRAANTHSS |
| | AKPCGQQSIHLGGVFELQPGASVFVNVTDPSQVSHGTGFTSFGLLKL |
| SEQ ID NO: 690 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to CD70 | QLYFDYWGQGTLVTVSSASTKGPSVFPLAPCSRSTSESTAALGCLVKDYFP |
| ECD | EPVTVSWNSGALTSGVHTFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSQRFAQAQQQLPLESLGWDVAELQLNHTGPQQDPRLYWQGGPALGR |
| | SFLHGPELDKGQLRIHRDGIYMVHIQVTLAICSSTTASRHHPTTLAVGICSPA |
| | SRSISLLRLSFHQGCTIASQRLTPLARGDTLCTNLTGTLLPSRNTDETFFGVQ |
| | WVRP |
| SEQ ID NO: 691 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to GITRL | QLYFDYWGQGTLVTVSSASTKGPSVFPLAPCSRSTSESTAALGCLVKDYFP |
| ECD | EPVTVSWNSGALTSGVHTFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSQLETAKEPCMAKFGPLPSKWQMASSEPPCVNKVSDWKLEILQNGLY |
| | LIYGQVAPNANYNDVAPFEVRLYKNKDMIQTLTNKSKIQNVGGTYELHVG |
| | DTIDLIFNSEHQVLKNNTYWGIILLANPQFIS |
| SEQ ID NO: 692 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to ICOSL | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSDTQEKEVRAMVGSDVELSCACPEGSRFDLNDVYVYWQTSESKTVV |
| | TYHIPQNSSLENVDSRYRNRALMSPAGMLRGDFSLRLFNVTPQDEQKFHCL |
| | VLSQSLGFQEVLSVEVTLHVAANFSVPVVSAPHSPSQDELTFTCTSINGYPR |
| | PNVYWINKTDNSLLDQALQNDTVFLNMRGLYDVVSVLRIARTPSVNIGCCI |
| | ENVLLQQNLTVGSQTGNDIGERDKITENPVSTGEKNAATWS |
| SEQ ID NO: 693 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to IL12 | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSIWELKKDVYVVELDWYPDAPGEMVVLTCDTPEEDGITWTLDQSSEV |
| | LGSGKTLTIQVKEFGDAGQYTCHKGGEVLSHSLLLLHKKEDGIWSTDILKD |
| | QKEPKNKTFLRCEAKNYSGRFTCWWLTTISTDLTFSVKSSRGSSDPQGVTC |
| | GAATLSAERVRGDNKEYEYSVECQEDSACPAAEESLPIEVMVDAVHKLKY |
| | ENYTSSFFIRDIIKPDPPKNLQLKPLKNSRQVEVSWEYPDTWSTPHSYFSLTF |
| | CVQVQGKSKREKKDRVFTDKTSATVICRKNASISVRAQDRYYSSSWSEWA |
| | SVPCSGGGGSGGGGSGGGGSRNLPVATPDPGMFPCLHHSQNLLRAVSNML |
| | QKARQTLEFYPCTSEEIDHEDITKDKTSTVEACLPLELTKNESCLNSRETSFI |
| | TNGSCLASRKTSFMMALCLSSIYEDLKMYQVEFKTMNAKLLMDPKRQIFL |
| | DQNMLAVIDELMQALNFNSETVPQKSSLEEPDFYKTKIKLCILLHAFRIRAV |
| | TIDRVMSYLNAS |
| SEQ ID NO: 694 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to IL15 | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSNWVNVISDLKKIEDLIQSMHIDATLYTESDVHPSCKVTAMKCFLLEL |
| | QVISLESGDASIHDTVENLIILANNSLSSNGNVTESGCKECEELEEKNIKEFL |
| | QSFVHIVQMFINTS |
| SEQ ID NO: 695 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to LIGHT | QLYFDYWGQGTLVTVSSASTKGPSVFPLAPCSRSTSESTAALGCLVKDYFP |
| ECD | EPVTVSWNSGALTSGVHIFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSDGPAGSWEQLIQERRSHEVNPAAHLTGANSSLTGSGGPLLWETQLG |
| | LAFLRGLSYHDGALVVTKAGYYYIYSKVQLGGVGCPLGLASTITHGLYKR |
| | TPRYPEELELLVSQQSPCGRATSSSRVWWDSSFLGGVVHLEAGEKVVVRV |
| | LDERLVRLRDGTRSYFGAFMV |
| SEQ ID NO: 696 | QVQLVQSGAEVKKPGASVKVSCKASGYTFTRYGISWVRQAPGQGLEWMG |
| anti-IL17R HC | WISTYSGNTNYAQKLQGRVTMTTDTSTSTAYMELRSLRSDDTAVYYCARR |
| fused to OX40L | QLYFDYWGQGTLVTVSSASTKGPSVFPLAPCSRSTSESTAALGCLVKDYFP |
| ECD | EPVTVSWNSGALTSGVHTFPAVLQSSGLYSLSSVVTVPSSNFGTQTYTCNV |
| | DHKPSNTKVDKTVERKCCVECPPCPAPPVAGPSVFLFPPKPKDTLMISRTPE |
| | VTCVVVDVSHEDPEVQFNWYVDGVEVHNAKTKPREEQFNSTFRVVSVLT |
| | VVHQDWLNGKEYKCKVSNKGLPAPIEKTISKTKGQPREPQVYTLPPSREEM |
| | TKNQVSLTCLVKGFYPSDIAVEWESNGQPENNYKTTPPMLDSDGSFFLYSK |
| | LTVDKSRWQQGNVFSCSVMHEALHNHYTQKSLSLSPGKGGGGSGGGGSG |
| | GGGSQVSHRYPRIQSIKVQFTEYKKEKGFILTSQKEDEIMKVQNNSVIINCD |
| | GFYLISLKGYFSQEVNISLHYQKDEEPLFQLKKVRSVNSLMVASLTYKDKV |
| | YLNVTTDNTSLDDFHVNGGELILIHQNPGEFCVL |
| SEQ ID NO: 697 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to 41BBL | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSACPWAVSGARASPGSAASPRLREGPE |
| | LSPDDPAGLLDLRQGMFAQLVAQNVLLIDGPLSWYSDPGLAGVSLTGGLS |
| | YKEDTKELVVAKAGVYYVFFQLELRRVVAGEGSGSVSLALHLQPLRSAAG |
| | AAALALTVDLPPASSEARNSAFGFQGRLLHLSAGQRLGVHLHTEARARHA |
| | WQLTQGATVLGLFRVTPEIPAGLPSPRSE |
| SEQ ID NO: 698 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to CD30L | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQRTDSIPNSPDNVPLKGGNCSEDLLCI |
| | LKRAPFKKSWAYLQVAKHLNKTKLSWNKDGILHGVRYQDGNLVIQFPGL |
| | YFIICQLQFLVQCPNNSVDLKLELLINKHIKKQALVTVCESGMQTKHVYQN |
| | LSQFLLDYLQVNTTISVNVDTFQYIDTSTFPLENVLSIFLYSNSD |
| SEQ ID NO: 699 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to CD40L | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSGDQNPQIAAHVISEASSKTTSVLQWA |
| | EKGYYTMSNNLVTLENGKQLTVKRQGLYYIYAQVTFCSNREASSQAPFIAS |
| | LCLKSPGRFERILLRAANTHSSAKPCGQQSIHLGGVFELQPGASVFVNVTDP |
| | SQVSHGTGFTSFGLLKL |
| SEQ ID NO: 700 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to CD70 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQRFAQAQQQLPLESLGWDVAELQLN |
| | HTGPQQDPRLYWQGGPALGRSFLHGPELDKGQLRIHRDGIYMVHIQVTLAI |
| | CSSTTASRHHPTTLAVGICSPASRSISLLRLSFHQGCTIASQRLTPLARGDTLC |
| | TNLTGTLLPSRNTDEITTGVQWVRP |
| SEQ ID NO: 701 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to GITRL | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQLETAKEPCMAKFGPLPSKWQMASSE |
| | PPCVNKVSDWKLEILQNGLYLIYGQVAPNANYNDVAPFEVRLYKNKDMIQ |
| | TLTNKSKIQNVGGTYELHVGDTIDLIFNSEHQVLKNNTYWGIILLANPQFIS |
| SEQ ID NO: 702 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to ICOSL | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSDTQEKEVRAMVGSDVELSCACPEGSR |
| | FDLNDVYVYWQTSESKTVVTYHIPQNSSLENVDSRYRNRALMSPAGMLRG |
| | DFSLRLFNVTPQDEQKFHCLVLSQSLGFQEVLSVEVTLHVAANFSVPVVSA |
| | PHSPSQDELTFTCTSINGYPRPNVYWINKTDNSLLDQALQNDTVFLNMRGL |
| | YDVVSVLRIARTPSVNIGCCIENVLLQQNLTVGSQTGNDIGERDKITENPVS |
| | TGEKNAATWS |
| SEQ ID NO: 703 | EIVMTQSPATLSVSPGERATLSCRASQSVSSNLAWFQQKPGQAPRPLIYDAS |
| anti- IL17R LC | TRATGVPARFSGSGSGTDFTLTISSLQSEDFAVYYCQQYDNWPLTFGGGTK |
| fused to IL12 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSIWELKKDVYVVELDWYPDAPGEMVV |
| | LTCDTPEEDGITWTLDQSSEVLGSGKTLTIQVKEFGDAGQYTCHKGGEVLS |
| | HSLLLLHKKEDGIWSTDILKDQKEPKNKIFLRCEAKNYSGRFTCWWLTTIS |
| | TDLTFSVKSSRGSSDPQGVTCGAATLSAERVRGDNKEYEYSVECQEDSACP |
| | AAEESLPIEVMVDAVHKLKYENYTSSFFIRDIIKPDPPKNLQLKPLKNSRQV |
| | EVSWEYPDTWSTPHSYFSLTFCVQVQGKSKREKKDRVFTDKTSATVICRK |
| | NASISVRAQDRYYSSSWSEWASVPCSGGGGSGGGGSGGGGSRNLPVATPD |
| | PGMFPCLHHSQNLLRAVSNMLQKARQTLEFYPCTSEEIDHEDITKDKTSTV |
| | EACLPLELTKNESCLNSRETSFITNGSCLASRKTSFMMALCLSSIYEDLKMY |
| | QVEFKTMNAKLLMDPKRQIFLDQNMLAVIDELMQALNFNSETVPQKSSLE |
| | EPDFYKTKIKLCILLHAFRIRAVTIDRVMSYLNAS |
| SEQ ID NO: 704 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to IL15 | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSNWVNVISDLKKIEDLIQSMHIDATLYT |
| | ESDVHPSCKVTAMKCFLLELQVISLESGDASIHDTVENLIILANNSLSSNGN |
| | VTESGCKECEELEEKNIKEFLQSFVHIVQMFINTS |
| SEQ ID NO: 705 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to LIGHT | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSDGPAGSWEQLIQERRSHEVNPAAHLT |
| | GANSSLTGSGGPLLWETQLGLAFLRGLSYHDGALVVTKAGYYYIYSKVQL |
| | GGVGCPLGLASTITHGLYKRTPRYPEELELLVSQQSPCGRATSSSRVWWDS |
| | SFLGGVVHLEAGEKVVVRVLDERLVRLRDGTRSYFGAFMV |
| SEQ ID NO: 706 | EIVMTQSPATLSVSPGERATLSCRASQSVSSNLAWFQQKPGQAPRPLIYDAS |
| anti-IL17R LC | TRATGVPARFSGSGSGTDFTLTISSLQSEDFAVYYCQQYDNWPLTFGGGTK |
| fused to OX40L | VEIKRTVAAPSVFIFPPSDEQLKSGTASVVCLLNNFYPREAKVQWKVDNAL |
| ECD | QSGNSQESVTEQDSKDSTYSLSSTLTLSKADYEKHKVYACEVTHQGLSSPV |
| | TKSFNRGECGGGGSGGGGSGGGGSQVSHRYPRIQSIKVQFTEYKKEKGFIL |
| | TSQKEDEIMKVQNNSVIINCDGFYLISLKGYFSQEVNISLHYQKDEEPLFQL |
| | KKVRSVNSLMVASLTYKDKVYLNVTTDNTSLDDFHVNGGELILIHQNPGE |
| | FCVL |
| SEQ ID NO: 707 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to 41BBL | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSACPWAVSGARASPGSAASPRLREGPELSPDDPAGL |
| | LDLRQGMFAQLVAQNVLLIDGPLSWYSDPGLAGVSLTGGLSYKEDTKELV |
| | VAKAGVYYVFFQLELRRVVAGEGSGSVSLALHLQPLRSAAGAAALALTVD |
| | LPPASSEARNSAFGFQGRLLHLSAGQRLGVHLHTEARARHAWQLTQGATV |
| | LGLFRVTPEIPAGLPSPRSE |
| SEQ ID NO: 708 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to CD30L | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSQRTDSIPNSPDNVPLKGGNCSEDLLCILKRAPFKKS |
| | WAYLQVAKHLNKTKLSWNKDGILHGVRYQDGNLVIQFPGLYFIICQLQFL |
| | VQCPNNSVDLKLELLINKHIKKQALVTVCESGMQTKHVYQNLSQFLLDYL |
| | QVNTTISVNVDTFQYIDTSTFPLENVLSIFLYSNSD |
| SEQ ID NO: 709 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to CD40L | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSGDQNPQIAAHVISEASSKTTSVLQWAEKGYYTMSN |
| | NLVTLENGKQLTVKRQGLYYIYAQVTFCSNREASSQAPFIASLCLKSPGRFE |
| | RILLRAANTHSSAKPCGQQSIHLGGVFELQPGASVFVNVTDPSQVSHGTGFT |
| | SFGLLKL |
| SEQ ID NO: 710 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to CD70 | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSQRFAQAQQQLPLESLGWDVAELQLNHTGPQQDPR |
| | LYWQGGPALGRSFLHGPELDKGQLRIHRDGIYMVHIQVTLAICSSTTASRH |
| | HPTTLAVGICSPASRSISLLRLSFHQGCTIASQRLTPLARGDTLCTNLTGTLLP |
| | SRNTDETFFGVQWVRP |
| SEQ ID NO: 711 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to GITRL | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSQLETAKEPCMAKFGPLPSKWQMASSEPPCVNKVS |
| | DWKLEILQNGLYLIYGQVAPNANYNDVAPFEVRLYKNKDMIQTLTNKSKI |
| | QNVGGTYELHVGDTIDLIFNSEHQVLKNNTYWGIILLANPQFIS |
| SEQ ID NO: 712 | EVQLVESGGGLVQPGGSLRLSCAASGYTFTNYGMNWVRQAPGKGLEWVG |
| anfi-VEGF HC | WINTYTGEPTYAADFKRRFTFSLDTSKSTAYLQMNSLRAEDTAVYYCAKY |
| fused to ICOSL | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKFIPPV |
| | LDSDGSFFLYSKLTVDKSRWQQGNVFSCSVMHEALHNHYTQKSLSLSPGK |
| | GGGGSGGGGSGGGGSDTQEKEVRAMVGSDVELSCACPEGSRFDLNDVYV |
| | YWQTSESKTVVTYHIPQNSSLENVDSRYRNRALMSPAGMLRGDFSLRLFN |
| | VTPQDEQKFHCLVLSQSLGFQEVLSVEVTLHVAANFSVPVVSAPHSPSQDE |
| | LTFTCTSINGYPRPNVYWINKTDNSLLDQALQNDTVFLNMRGLYDVVSVL |
| | RIARTPSVNIGCCIENVLLQQNLTVGSQTGNDIGERDKITENPVSTGEKNAA |
| | TWS |
| | EVQLVESGGGLVQPGGSLRLSCAASGYTFTNYGMNWVRQAPGKGLEWVG |
| SEQ ID NO: 713 | WINTYTGEPTYAADFKRRFTFSLDTSKSTAYLQMNSLRAEDTAVYYCAKY |
| anti-VEGF HC | PHYYGSSHWYFDVWGQGTLVTVSSASTKGPSVFPLAPSSKSTSGGTAALG |
| fused to IL12 | CLVKDYFPEPVTVSWNSGALTSGVHTFPAVLQSSGLYSLSSVVTVPSSSLGT |
| ECD | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSIWELKKDVYVVELDWYPDAPGEMVVLTCDTPEED |
| | GITWTLDQSSEVLGSGKTLTIQVKEFGDAGQYTCHKGGEVLSHSLLLLHKK |
| | EDGIWSTDILKDQKEPKNKTFLRCEAKNYSGRFTCWWLTTISTDLTFSVKSS |
| | RGSSDPQGVTCGAATLSAERVRGDNKEYEYSVECQEDSACPAAEESLPIEV |
| | MVDAVHKLKYENYTSSFFIRDIIKPDPPKNLQLKPLKNSRQVEVSWEYPDT |
| | WSTPHSYFSLTFCVQVQGKSKREKKDRVFTDKTSATVICRKNASISVRAQD |
| | RYYSSSWSEWASVPCSGGGGSGGGGSGGGGSRNLPVATPDPGMFPCLHHS |
| | QNLLRAVSNMLQKARQTLEFYPCTSEEIDHEDITKDKTSTVEACLPLELTKN |
| | ESCLNSRETSFITNGSCLASRKTSFMMALCLSSIYEDLKMYQVEFKTMNAK |
| | LLMDPKRQIFLDQNMLAVIDELMQALNFNSETVPQKSSLEEPDFYKTKIKL |
| | CILLHAFRIRAVTIDRVMSYLNAS |
| SEQ ID NO: 714 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to IL15 | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSNWVNVISDLKKIEDLIQSMHIDATLYTESDVHPSCK |
| | VTAMKCFLLELQVISLESGDASIHDTVENLIILANNSLSSNGNVTESGCKECE |
| | ELEEKNIKEFLQSFVHIVQMFINTS |
| SEQ ID NO: 715 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to LIGHT | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSDGPAGSWEQLIQERRSHEVNPAAHLTGANSSLTGS |
| | GGPLLWETQLGLAFLRGLSYHDGALVVTKAGYYYIYSKVQLGGVGCPLGL |
| | ASTITHGLYKRTPRYPEELELLVSQQSPCGRATSSSRVWWDSSFLGGVVHL |
| | EAGEKVVVRVLDERLVRLRDGTRSYFGAFMV |
| SEQ ID NO: 716 | EVQLVESGGGLVQPGGSLRLSCAASGYTFTNYGMNWVRQAPGKGLEWVG |
| anti-VEGF HC | WINTYTGEPTYAADFKRRFTFSLDTSKSTAYLQMNSLRAEDTAVYYCAKY |
| fused to OX40L | PHYYGSSHWYFDVWGQGTLVTVSSASTKGPSVFPLAPSSKSTSGGTAALG |
| ECD | CLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | GGGGSGGGGSGGGGSQVSHRYPRIQSIKVQFTEYKKEKGFILTSQKEDEIM |
| | KVQNNSVIINCDGFYLISLKGYFSQEVNISLHYQKDEEPLFQLKKVRSVNSL |
| | MVASLTYKDKVYLNVTTDNTSLDDFHVNGGELILIHQNPGEFCVL |
| SEQ ID NO: 717 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to 41BBL | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSACPWAVSGARASPGSAASPRLREGPEL |
| | SPDDPAGLLDLRQGMFAQLVAQNVLLIDGPLSWYSDPGLAGVSLTGGLSY |
| | KEDTKELVVAKAGVYYVFFQLELRRVVAGEGSGSVSLALHLQPLRSAAGA |
| | AALALTVDLPPASSEARNSAFGFQGRLLHLSAGQRLGVHLHTEARARHAW |
| | QLTQGATVLGLFRVTPEIPAGLPSPRSE |
| SEQ ID NO: 718 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to CD30L | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSQRTDSIPNSPDNVPLKGGNCSEDLLCIL |
| | KRAPFKKSWAYLQVAKHLNKTKLSWNKDGILHGVRYQDGNLVIQFPGLY |
| | FIICQLQFLVQCPNNSVDLKLELLINKHIKKQALVTVCESGMQTKHVYQNL |
| | SQFLLDYLQVNTTISVNVDTFQYIDTSTFPLENVLSIFLYSNSD |
| SEQ ID NO: 719 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to CD40L | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSGDQNPQIAAHVISEASSKTTSVLQWAE |
| | KGYYTMSNNLVTLENGKQLTVKRQGLYYIYAQVTFCSNREASSQAPFIASL |
| | CLKSPGRFERILLRAANTHSSAKPCGQQSIHLGGVFELQPGASVFVNVTDPS |
| | QVSHGTGFTSFGLLKL |
| SEQ ID NO: 720 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to CD70 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSQRFAQAQQQLPLESLGWDVAELQLNH |
| | TGPQQDPRLYWQGGPALGRSFLHGPELDKGQLRIHRDGIYMVHIQVTLAIC |
| | SSTTASRHHPTTLAVGICSPASRSISLLRLSFHQGCTIASQRLTPLARGDTLCT |
| | NLTGTLLPSRNTDETFFGVQWVRP |
| SEQ ID NO: 721 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to GITRL | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSQLETAKEPCMAKFGPLPSKWQMASSEP |
| | PCVNKVSDWKLEILQNGLYLIYGQVAPNANYNDVAPFEVRLYKNKDMIQT |
| | LTNKSKIQNVGGTYELHVGDTIDLIFNSEHQVLKNNTYWGIILLANPQFIS |
| SEQ ID NO: 722 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to ICOSL | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSDTQEKEVRAMVGSDVELSCACPEGSRF |
| | DLNDVYVYWQTSESKTVVTYHIPQNSSLENVDSRYRNRALMSPAGMLRG |
| | DFSLRLFNVTPQDEQKFHCLVLSQSLGFQEVLSVEVTLHVAANFSVPVVSA |
| | PHSPSQDELTFTCTSINGYPRPNVYWINKTDNSLLDQALQNDTVFLNMRGL |
| | YDVVSVLRIARTPSVNIGCCIENVLLQQNLTVGSQTGNDIGERDKITENPVS |
| | TGEKNAATWS |
| SEQ ID NO: 723 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to IL12 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSIWELKKDVYVVELDWYPDAPGEMVVL |
| | TCDTPEEDGITWTLDQSSEVLGSGKTLTIQVKEFGDAGQYTCHKGGEVLSH |
| | SLLLLHKKEDGIWSTDILKDQKEPKNKITLRCEAKNYSGRFTCWWLTTIST |
| | DLTFSVKSSRGSSDPQGVTCGAATLSAERVRGDNKEYEYSVECQEDSACPA |
| | AEESLPIEVMVDAVHKLKYENYTSSFFIRDIIKPDPPKNLQLKPLKNSRQVE |
| | VSWEYPDTWSTPHSYFSLTFCVQVQGKSKREKKDRVFTDKTSATVICRKN |
| | ASISVRAQDRYYSSSWSEWASVPCSGGGGSGGGGSGGGGSRNLPVATPDP |
| | GMFPCLHHSQNLLRAVSNMLQKARQTLEFYPCTSEEIDHEDITKDKTSTVE |
| | ACLPLELTKNESCLNSRETSFITNGSCLASRKTSFMMALCLSSIYEDLKMYQ |
| | VEFKTMNAKLLMDPKRQIFLDQNMLAVIDELMQALNFNSETVPQKSSLEEP |
| | DFYKTKIKLCILLHAFRIRAVTIDRVMSYLNAS |
| SEQ ID NO: 724 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to IL15 | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSNWVNVISDLKKIEDLIQSMHIDATLYTE |
| | SDVHPSCKVTAMKCFLLELQVISLESGDASIHDTVENLIILANNSLSSNGNV |
| | TESGCKECEELEEKNIKEFLQSFVHIVQMFINTS |
| SEQ ID NO: 725 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to LIGHT | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSDGPAGSWEQLIQERRSHEVNPAAHLTG |
| | ANSSLTGSGGPLLWETQLGLAFLRGLSYHDGALVVTKAGYYYIYSKVQLG |
| | GVGCPLGLASTITHGLYKRTPRYPEELELLVSQQSPCGRATSSSRVWWDSS |
| | FLGGVVHLEAGEKVVVRVLDERLVRLRDGTRSYFGAFMV |
| SEQ ID NO: 726 | DIQMTQSPSSLSASVGDRVTITCSASQDISNYLNWYQQKPGKAPKVLIYFTS |
| anti-VEGF LC | SLHSGVPSRFSGSGSGTDFTLTISSLQPEDFATYYCQQYSTVPWTFGQGTKV |
| fused to OX40L | EIKRTVAAPSVFIFPPSDEQLKSGTASVVCLLNNFYPREAKVQWKVDNALQ |
| ECD | SGNSQESVTEQDSKDSTYSLSSTLTLSKADYEKHKVYACEVTHQGLSSPVT |
| | KSFNRGECGGGGSGGGGSGGGGSQVSHRYPRIQSIKVQFTEYKKEKGFILT |
| | SQKEDEIMKVQNNSVIINCDGFYLISLKGYFSQEVNISLHYQKDEEPLFQLK |
| | KVRSVNSLMVASLTYKDKVYLNVTTDNTSLDDFHVNGGELILIHQNPGEFC |
| | VL |
| SEQ ID NO: 727 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-Fc- | GGTKLEIKGGGGSGGGGSGGGGSQVQLQQSGAELVRPGSSVKISCKASGY |
| BTLAecd | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSKESCDVQLYIKRQS |
| | EHSILAGDPFELECPVKYCANRPHVTWCKLNGTTCVKLEDRQTSWKEEKNI |
| | SFFILHFEPVLPNDNGSYRCSANFQSNLIESHSTTLYVTDVKSASERPSKDEM |
| | AS |
| SEQ ID NO: 728 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-BTLAecd | GGTKLEIKGGGGSGGGGSGGGGSQVQLQQSGAELVRPGSSVKISCKASGY |
| | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKGGGG |
| | SGGGGSGGGGSKESCDVQLYIKRQSEHSILAGDPFELECPVKYCANRPHVT |
| | WCKLNGTTCVKLEDRQTSWKEEKNISFFILHFEPVLPNDNGSYRCSANFQS |
| | NLIESHSTTLYVTDVKSASERPSKDEMASHHHHHH |
| SEQ ID NO: 729 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-Fc- | LLRHGAMDYWGQGTLVTVSSGGGGSGGGGSGGGGSDIQMTQSPSSLSASV |
| BTLAecd | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSKESCDVQLYIKRQS |
| | EHSILAGDPFELECPVKYCANRPHVTWCKLNGTTCVKLEDRQTSWKEEKNI |
| | SFFILHFEPVLPNDNGSYRCSANFQSNLIESHSTTLYVTDVKSASERPSKDEM |
| | AS |
| SEQ ID NO: 730 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-BTLAecd | LLRHGAMDYWGQGTLVTVSSGGGGSGGGGSGGGGSDIQMTQSPSSLSASV |
| | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLGGGG |
| | SGGGGSGGGGSKESCDVQLYIKRQSEHSILAGDPFELECPVKYCANRPHVT |
| | WCKLNGTTCVKLEDRQTSWKEEKNISFFILHFEPVLPNDNGSYRCSANFQS |
| | NLIESHSTTLYVTDVKSASERPSKDEMASHHHHHH |
| SEQ ID NO: 731 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-Fc-PD1ecd | GGTKLEIKGGGGSGGGGSGGGGSQVQLQQSGAELVRPGSSVKISCKASGY |
| | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSPGWFLDSPDRPWN |
| | PPTFSPALLVVTEGDNATFTCSFSNTSESFVLNWYRMSPSNQTDKLAAFPED |
| | RSQPGQDCRFRVTQLPNGRDFHMSVVRARRNDSGTYLCGAISLAPKAQIKE |
| | SLRAELRVTERRAEVPTAHPSPSPRPAGQFQTLV |
| SEQ ID NO: 732 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-PD1ecd | GGTKLEIKGGGGSGGGGSGGGGSQVQLQQSGAELVRPGSSVKISCKASGY |
| | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKGGGG |
| | SGGGGSGGGGSPGWFLDSPDRPWNPPTFSPALLVVTEGDNATFTCSFSNTS |
| | ESFVLNWYRMSPSNQTDKLAAFPEDRSQPGQDCRFRVTQLPNGRDFHMSV |
| | VRARRNDSGTYLCGAISLAPKAQIKESLRAELRVTERRAEVPTAHPSPSPRP |
| | AGQFQTLVHHHHHH |
| SEQ ID NO: 733 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-Fc-PD1ecd | LLRHGAMDYWGQGTLVTVSSGGGGSGGGGSGGGGSDIQMTQSPSSLSASV |
| | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSPGWFLDSPDRPWN |
| | PPTFSPALLVVTEGDNATFTCSFSNTSESFVLNWYRMSPSNQTDKLAAFPED |
| | RSQPGQDCRFRVTQLPNGRDFHMSVVRARRNDSGTYLCGAISLAPKAQIKE |
| | SLRAELRVTERRAEVPTAHPSPSPRPAGQFQTLV |
| SEQ ID NO: 734 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-PD1ecd | LLRHGAMDYWGQGTLVTVSSGGGGSGGGGSGGGGSDIQMTQSPSSLSASV |
| | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLGGGG |
| | SGGGGSGGGGSPGWFLDSPDRPWNPPTFSPALLVVTEGDNATFTCSFSNTS |
| | ESFVLNWYRMSPSNQTDKLAAFPEDRSQPGQDCRFRVTQLPNGRDFHMSV |
| | VRARRNDSGTYLCGAISLAPKAQIKESLRAELRVTERRAEVPTAHPSPSPRP |
| | AGQFQTLVHHHHHH |
| SEQ ID NO: 735 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-Fc- | GGTKLEIKGGGGSGGGGSGGGGSQVQLQQSGAELVRPGSSVKISCKASGY |
| SIGLEC10ecd | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSMDGRFWIRVQESV |
| | MVPEGLCISVPCSFSYPRQDWTGSTPAYGYWFKAVTETTKGAPVATNHQS |
| | REVEMSTRGRFQLTGDPAKGNCSLVIRDAQMQDESQYFFRVERGSYVRYN |
| | FMNDGFFLKVTALTQKPDVYIPETLEPGQPVTVICVFNWAFEECPPPSFSWT |
| | GAALSSQGTKPTTSHFSVLSFTPRPQDHNTDLTCHVDFSRKGVSAQRTVRL |
| | RVAYAPRDLVISISRDNTPALEPQPQGNVPYLEAQKGQFLRLLCAADSQPP |
| | ATLSWVLQNRVLSSSHPWGPRPLGLELPGVKAGDSGRYTCRAENRLGSQQ |
| | RALDLSVQYPPENLRVMVSQANRTVLENLGNGTSLPVLEGQSLCLVCVTH |
| | SSPPARLSWTQRGQVLSPSQPSDPGVLELPRVQVEHEGEFTCHARHPLGSQ |
| | HVSLSLSVHYSPKLLGPSCSWEAEGLHCSCSSQASPAPSLRWWLGEELLEG |
| | NSSQDSFEVTPSSAGPWANSSLSLHGGLSSGLRLRCEAWNVHGAQSGSILQ |
| | LPDKKGLIST |
| SEQ ID NO: 736 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE- | GGTKLEIKGGGGSGGGGSGGGGSQVQLQQSGAELVRPGSSVKISCKASGY |
| SIGLEC10ecd | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKGGGG |
| | SGGGGSGGGGSMDGRFWIRVQESVMVPEGLCISVPCSFSYPRQDWTGSTP |
| | AYGYWFKAVTETTKGAPVATNHQSREVEMSTRGRFQLTGDPAKGNCSLVI |
| | RDAQMQDESQYFFRVERGSYVRYNFMNDGFFLKVTALTQKPDVYIPETLE |
| | PGQPVTVICVFNWAFEECPPPSFSWTGAALSSQGTKPTTSHFSVLSFTPRPQ |
| | DHNTDLTCHVDFSRKGVSAQRTVRLRVAYAPRDLVISISRDNTPALEPQPQ |
| | GNVPYLEAQKGQFLRLLCAADSQPPATLSWVLQNRVLSSSHPWGPRPLGL |
| | ELPGVKAGDSGRYTCRAENRLGSQQRALDLSVQYPPENLRVMVSQANRTV |
| | LENLGNGTSLPVLEGQSLCLVCVTHSSPPARLSWTQRGQVLSPSQPSDPGV |
| | LELPRVQVEHEGEFTCHARHPLGSQHVSLSLSVHYSPKLLGPSCSWEAEGL |
| | HCSCSSQASPAPSLRWWLGEELLEGNSSQDSFEVTPSSAGPWANSSLSLHG |
| | GLSSGLRLRCEAWNVHGAQSGSILQLPDKKGLISTHHHHHH |
| SEQ ID NO: 737 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-Fc- | LLRHGAMDYWGQGTLVTVSSGGGGSGGGGSGGGGSDIQMTQSPSSLSASV |
| SIGLEC10ecd | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSMDGRFWIRVQESV |
| | MVPEGLCISVPCSFSYPRQDWTGSTPAYGYWFKAVTETTKGAPVATNHQS |
| | REVEMSTRGRFQLTGDPAKGNCSLVIRDAQMQDESQYFFRVERGSYVRYN |
| | FMNDGFFLKVTALTQKPDVYIPETLEPGQPVTVICVFNWAFEECPPPSFSWT |
| | GAALSSQGTKPTTSHFSVLSFTPRPQDHNTDLTCHVDFSRKGVSAQRTVRL |
| | RVAYAPRDLVISISRDNTPALEPQPQGNVPYLEAQKGQFLRLLCAADSQPP |
| | ATLSWVLQNRVLSSSHPWGPRPLGLELPGVKAGDSGRYTCRAENRLGSQQ |
| | RALDLSVQYPPENLRVMVSQANRTVLENLGNGTSLPVLEGQSLCLVCVTH |
| | SSPPARLSWTQRGQVLSPSQPSDPGVLELPRVQVEHEGEFTCHARHPLGSQ |
| | HVSLSLSVHYSPKLLGPSCSWEAEGLHCSCSSQASPAPSLRWWLGEELLEG |
| | NSSQDSFEVTPSSAGPWANSSLSLHGGLSSGLRLRCEAWNVHGAQSGSILQ |
| | LPDKKGLIST |
| SEQ ID NO: 738 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE- | LLRHGAMDYWGQGTLVTVSSGGGGSGGGGSGGGGSDIQMTQSPSSLSASV |
| SIGLEC10ecd | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLGGGG |
| | SGGGGSGGGGSMDGRFWIRVQESVMVPEGLCISVPCSFSYPRQDWTGSTP |
| | AYGYWFKAVTETTKGAPVATNHQSREVEMSTRGRFQLTGDPAKGNCSLVI |
| | RDAQMQDESQYFFRVERGSYVRYNFMNDGFFLKVTALTQKPDVYIPETLE |
| | PGQPVTVICVFNWAFEECPPPSFSWTGAALSSQGTKPTTSHFSVLSFTPRPQ |
| | DHNTDLTCHVDFSRKGVSAQRTVRLRVAYAPRDLVISISRDNTPALEPQPQ |
| | GNVPYLEAQKGQFLRLLCAADSQPPATLSWVLQNRVLSSSHPWGPRPLGL |
| | ELPGVKAGDSGRYTCRAENRLGSQQRALDLSVQYPPENLRVMVSQANRTV |
| | LENLGNGTSLPVLEGQSLCLVCVTHSSPPARLSWTQRGQVLSPSQPSDPGV |
| | LELPRVQVEHEGEFTCHARHPLGSQHVSLSLSVHYSPKLLGPSCSWEAEGL |
| | HCSCSSQASPAPSLRWWLGEELLEGNSSQDSFEVTPSSAGPWANSSLSLHG |
| | GLSSGLRLRCEAWNVHGAQSGSILQLPDKKGLISTHHHHHH |
| SEQ ID NO: 739 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-Fc- | GGTKLEIKGGGGSGGGGSGGGGSQVQLQQSGAELVRPGSSVKISCKASGY |
| SIRPaecd | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSEEELQVIQPDKSVL |
| | VAAGETATLRCTATSLIPVGPIQWFRGAGPGRELIYNQKEGHFPRVTTVSDL |
| | TKRNNMDFSIRIGNITPADAGTYYCVKFRKGSPDDVEFKSGAGTELSVRAK |
| | PS |
| SEQ ID NO: 740 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-SIRPaecd | GGTKLEIKGGGGSGGGGSGGGGSQVQLQQSGAELVRPGSSVKISCKASGY |
| | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKGGGG |
| | SGGGGSGGGGSEEELQVIQPDKSVLVAAGETATLRCTATSLIPVGPIQWFRG |
| | AGPGRELIYNQKEGHFPRVTTVSDLTKRNNMDFSIRIGNITPADAGTYYCV |
| | KFRKGSPDDVEFKSGAGTELSVRAKPSHHHHHH |
| SEQ ID NO: 741 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-Fc- | LLRHGAMDYWGQGTLVTVSSGGGGSGGGGSGGGGSDIQMTQSPSSLSASV |
| SIRPaecd | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSEEELQVIQPDKSVL |
| | VAAGETATLRCTATSLIPVGPIQWFRGAGPGRELIYNQKEGHFPRVTTVSDL |
| | TKRNNMDFSIRIGNITPADAGTYYCVKFRKGSPDDVEFKSGAGTELSVRAK |
| | PS |
| SEQ ID NO: 742 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-SIRPaecd | LLRHGAMDYWGQGTLVTVSSGGGGSGGGGSGGGGSDIQMTQSPSSLSASV |
| | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLGGGG |
| | SGGGGSGGGGSEEELQVIQPDKSVLVAAGETATLRCTATSLIPVGPIQWFRG |
| | AGPGRELIYNQKEGHFPRVTTVSDLTKRNNMDFSIRIGNITPADAGTYYCV |
| | KFRKGSPDDVEFKSGAGTELSVRAKPSHHHHHH |
| SEQ ID NO: 743 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-Fc- | GGTKLEIKGGGGSGGGGSGGGGSQVQLQQSGAELVRPGSSVKISCKASGY |
| TIM3ecd | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSSEVEYRAEVGQNA |
| | YLPCFYTPAAPGNLVPVCWGKGACPVFECGNVVLRTDERDVNYWTSRYW |
| | LNGDFRKGDVSLTIENVTLADSGIYCCRIQIPGIMNDEKFNLKLVIKPAKVTP |
| | APTRQRDFTAAFPRMLTTRGHGPAETQTLGSLPDINLTQISTLANELRDSRL |
| | ANDLRDSGATIRIG |
| SEQ ID NO: 744 | DIQLTQSPASLAVSLGQRATISCKASQSVDYDGDSYLNWYQQIPGQPPKLLI |
| CD3/CD19 | YDASNLVSGIPPRFSGSGSGTDFTLNIHPVEKVDAATYHCQQSTEDPWTFG |
| BiTE-TIM3ecd | GGTKLEIKGGGGSGGGGSGGGGSQVQLQQSGAELVRPGSSVKISCKASGY |
| | AFSSYWMNWVKQRPGQGLEWIGQIWPGDGDTNYNGKFKGKATLTADESS |
| | STAYMQLSSLASEDSAVYFCARRETTTVGRYYYAMDYWGQGTTVTVSSG |
| | GGGSDIKLQQSGAELARPGASVKMSCKTSGYTFTRYTMHWVKQRPGQGL |
| | EWIGYINPSRGYTNYNQKFKDKATLTTDKSSSTAYMQLSSLTSEDSAVYYC |
| | ARYYDDHYCLDYWGQGTTLTVSSVEGGSGGSGGSGGSGGVDDIQLTQSPA |
| | IMSASPGEKVTMTCRASSSVSYMNWYQQKSGTSPKRWIYDTSKVASGVPY |
| | RFSGSGSGTSYSLTISSMEAEDAATYYCQQWSSNPLTFGAGTKLELKGGGG |
| | SGGGGSGGGGSSEVEYRAEVGQNAYLPCFYTPAAPGNLVPVCWGKGACP |
| | VFECGNVVLRTDERDVNYWTSRYWLNGDFRKGDVSLTIENVTLADSGIYC |
| | CRIQIPGIMNDEKFNLKLVIKPAKVTPAPTRQRDFTAAFPRMLTTRGHGPAE |
| | TQTLGSLPDINLTQISTLANELRDSRLANDLRDSGATIRIGHHHHHH |
| SEQ ID NO: 745 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-Fc- | LLRHGAMDYWGQGTLVTVSSGGGGSGGGGSGGGGSDIQMTQSPSSLSASV |
| TIM3ecd | GDRVTITCKASQNVDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLASTK |
| | GPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCS |
| | VMHEALHNHYTQKSLSLSPGKGGGGSGGGGSGGGGSSEVEYRAEVGQNA |
| | YLPCFYTPAAPGNLVPVCWGKGACPVFECGNVVLRTDERDVNYWTSRYW |
| | LNGDFRKGDVSLTIENVTLADSGIYCCRIQIPGIMNDEKFNLKLVIKPAKVTP |
| | APTRQRDFTAAFPRMLTTRGHGPAETQTLGSLPDINLTQISTLANELRDSRL |
| | ANDLRDSGATIRIG |
| SEQ ID NO: 746 | QVQLVESGGGLVKPGESLRLSCAASGFTFSDYYMYWVRQAPGKGLEWVA |
| CD3/PSMA | IISDGGYYTYYSDIIKGRFTISRDNAKNSLYLQMNSLKAEDTAVYYCARGFP |
| BiTE-TIM3ecd | LLRHGAMDYWGQGTLVTVSSGGGGSGGGGSGGGGSDIQMTQSPSSLSASV |
| | GDRVTITCKASQNvDTNVAWYQQKPGQAPKSLIYSASYRYSDVPSRFSGSA |
| | SGTDFTLTISSVQSEDFATYYCQQYDSYPYTFGGGTKLEIKSGGGGSEVQLV |
| | ESGGGLVQPGGSLKLSCAASGFTFNKYAMNWVRQAPGKGLEWVARIRSK |
| | YNNYATYYADSVKDRFTISRDDSKNTAYLQMNNLKTEDTAVYYCVRHGN |
| | FGNSYISYWAYWGQGTLVTVSSGGGGSGGGGSGGGGSQTVVTQEPSLTVS |
| | PGGTVTLTCGSSTGAVTSGNYPNWVQQKPGQAPRGLIGGTKFLAPGTPARF |
| | SGSLLGGKAALTLSGVQPEDEAEYYCVLWYSNRWVFGGGTKLTVLGGGG |
| | SGGGGSGGGGSSEVEYRAEVGQNAYLPCFYTPAAPGNLVPVCWGKGACP |
| | VFECGNVVLRTDERDVNYWTSRYWLNGDFRKGDVSLTIENVTLADSGIYC |
| | CRIQIPGIMNDEKFNLKLVIKPAKVTPAPTRQRDFTAAFPRMLTTRGHGPAE |
| | TQTLGSLPDINLTQISTLANELRDSRLANDLRDSGATIRIGHHHHHH |
| SEQ ID NO: 747 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC1-BTLAecd | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | GGGGSGGGGSGGGGSKESCDVQLYIKRQSEHSILAGDPFELECPVKYCANR |
| | PHVTWCKLNGTTCVKLEDRQTSWKEEKNISFFILHEEPVLPNDNGSYRCSA |
| | NFQSNLIESHSTTLYVTDVKSASERPSKDEMAS |
| SEQ ID NO: 748 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC2-BTLAecd | LVKDYFPEPVTVSWNSGALTSGVHTFPAVLQSSGLYSLSSVVTVPSSSLGTQ |
| | TYICNVNHKPSNTKVDKKVEPKSCDGGGGSGGGGSEVQLLESGGGLVQPG |
| | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSKESCDVQLYIKRQSEHSILAGDPFELECPVKYCANRPHVTWCK |
| | LNGTTCVKLEDRQTSWKEEKNISFFILHEEPVLPNDNGSYRCSANFQSNLIE |
| | SHSTTLYVTDVKSASERPSKDEMAS |
| SEQ ID NO: 749 | DIQMTQSPSSLSASVGDRVTITCKASAAVGTYVAWYQQKPGKAPKLLIYSA |
| a- | SYRKRGVPSRFSGSGSGTDFTLTISSLQPEDFATYYCHQYYTYPLFTFGQGT |
| CEACAM5/CD3 | KLEIKRTVAAPSVFIFPPSDEQLKSGTASVVCLLNNFYPREAKVQWKVDNA |
| LC1-BTLAecd | LQSGNSQESVTEQDSKDSTYSLSSTLTLSKADYEKHKVYACEVTHQGLSSP |
| | VTKSFNRGECGGGGSGGGGSGGGGSKESCDVQLYIKRQSEHSILAGDPFEL |
| | ECPVKYCANRPHVTWCKLNGTTCVKLEDRQTSWKEEKNISFFILHFEPVLP |
| | NDNGSYRCSANFQSNLIESHSTTLYVTDVKSASERPSKDEMAS |
| SEQ ID NO: 750 | QAVVTQEPSLTVSPGGTVTLTCGSSTGAVTTSNYANWVQEKPGQAFRGLIG |
| a- | GTNKRAPGTPARFSGSLLGGKAALTLSGAQPEDEAEYYCALWYSNLWVFG |
| CEACAM5/CD3 | GGTKLTVLSSASTKGPSVFPLAPSSKSTSGGTAALGCLVKDYFPEPVTVSW |
| LC2-BTLAecd | NSGALTSGVHTFPAVLQSSGLYSLSSVVTVPSSSLGTQTYICNVNHKPSNTK |
| | VDKKVEPKSCGGGGSGGGGSGGGGSKESCDVQLYIKRQSEHSILAGDPFEL |
| | ECPVKYCANRPHVTWCKLNGTTCVKLEDRQTSWKEEKNISFFILHFEPVLP |
| | NDNGSYRCSANFQSNLIESHSTTLYVTDVKSASERPSKDEMAS |
| SEQ ID NO: 751 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC1-PD1ecd | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | GGGGSGGGGSGGGGSPGWFLDSPDRPWNPPTFSPALLVVTEGDNATFTCSF |
| | SNTSESFVLNWYRMSPSNQTDKLAAFPEDRSQPGQDCRFRVTQLPNGRDF |
| | HMSVVRARRNDSGTYLCGAISLAPKAQIKESLRAELRVTERRAEVPTAHPS |
| | PSPRPAGQFQTLV |
| SEQ ID NO: 752 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC2-PD1ecd | LVKDYFPEPVTVSWNSGALTSGVHTFPAVLQSSGLYSLSSVVTVPSSSLGTQ |
| | TYICNVNHKPSNTKVDKKVEPKSCDGGGGSGGGGSEVQLLESGGGLVQPG |
| | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSPGWFLDSPDRPWNPPTFSPALLVVTEGDNATFTCSFSNTSESFV |
| | LNWYRMSPSNQTDKLAAFPEDRSQPGQDCRFRVTQLPNGRDFHMSVVRA |
| | RRNDSGTYLCGAISLAPKAQIKESLRAELRVTERRAEVPTAHPSPSPRPAGQ |
| | FQTLV |
| SEQ ID NO: 753 | DIQMTQSPSSLSASVGDRVTITCKASAAVGTYVAWYQQKPGKAPKLLIYSA |
| a- | SYRKRGVPSRFSGSGSGTDFTLTISSLQPEDFATYYCHQYYTYPLFTFGQGT |
| CEACAM5/CD3 | KLEIKRTVAAPSVFIFPPSDEQLKSGTASVVCLLNNFYPREAKVQWKVDNA |
| LC1-PD1ecd | LQSGNSQESVTEQDSKDSTYSLSSTLTLSKADYEKHKVYACEVTHQGLSSP |
| | VTKSFNRGECGGGGSGGGGSGGGGSPGWFLDSPDRPWNPPTFSPALLVVT |
| | EGDNATFTCSFSNTSESFVLNWYRMSPSNQTDKLAAFPEDRSQPGQDCRFR |
| | VTQLPNGRDFHMSVVRARRNDSGTYLCGAISLAPKAQIKESLRAELRVTER |
| | RAEVPTAHPSPSPRPAGQFQTLV |
| SEQ ID NO: 754 | QAVVTQEPSLTVSPGGTVTLTCGSSTGAVTTSNYANWVQEKPGQAFRGLIG |
| a- | GTNKRAPGTPARFSGSLLGGKAALTLSGAQPEDEAEYYCALWYSNLWVFG |
| CEACAM5/CD3 | GGTKLTVLSSASTKGPSVFPLAPSSKSTSGGTAALGCLVKDYFPEPVTVSW |
| LC2-PD1ecd | NSGALTSGVHTFPAVLQSSGLYSLSSVVTVPSSSLGTQTYICNVNHKPSNTK |
| | VDKKVEPKSCGGGGSGGGGSGGGGSPGWFLDSPDRPWNPPTFSPALLVVT |
| | EGDNATFTCSFSNTSESFVLNWYRMSPSNQTDKLAAFPEDRSQPGQDCRFR |
| | VTQLPNGRDFHMSVVRARRNDSGTYLCGAISLAPKAQIKESLRAELRVTER |
| | RAEVPTAHPSPSPRPAGQFQTLV |
| SEQ ID NO: 755 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC1- | LVKDYFPEPVTVSWNSGALTSGVHTFPAVLQSSGLYSLSSVVTVPSSSLGTQ |
| SIGLEC10ecd | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | GGGGSGGGGSGGGGSMDGRFWIRVQESVMVPEGLCISVPCSFSYPRQDWT |
| | GSTPAYGYWFKAVTETTKGAPVATNHQSREVEMSTRGRFQLTGDPAKGN |
| | CSLVIRDAQMQDESQYFFRVERGSYVRYNFMNDGFFLKVTALTQKPDVYI |
| | PETLEPGQPVTVICVFNWAFEECPPPSFSWTGAALSSQGTKPTTSHFSVLSFT |
| | PRPQDHNTDLTCHVDFSRKGVSAQRTVRLRVAYAPRDLVISISRDNTPALE |
| | PQPQGNVPYLEAQKGQFLRLLCAADSQPPATLSWVLQNRVLSSSHPWGPR |
| | PLGLELPGVKAGDSGRYTCRAENRLGSQQRALDLSVQYPPENLRVMVSQA |
| | NRTVLENLGNGTSLPVLEGQSLCLVCVTHSSPPARLSWTQRGQVLSPSQPS |
| | DPGVLELPRVQVEHEGEFTCHARHPLGSQHVSLSLSVHYSPKLLGPSCSWE |
| | AEGLHCSCSSQASPAPSLRWWLGEELLEGNSSQDSFEVTPSSAGPWANSSL |
| | SLHGGLSSGLRLRCEAWNVHGAQSGSILQLPDKKGLIST |
| SEQ ID NO: 756 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC2- | LVKDYFPEPVTVSWNSGALTSGVHTFPAVLQSSGLYSLSSVVTVPSSSLGTQ |
| SIGLEC10ecd | TYICNVNHKPSNTKVDKKVEPKSCDGGGGSGGGGSEVQLLESGGGLVQPG |
| | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSMDGRFWIRVQESVMVPEGLCISVPCSFSYPRQDWTGSTPAYG |
| | YWFKAVTETTKGAPVATNHQSREVEMSTRGRFQLTGDPAKGNCSLVIRDA |
| | QMQDESQYFFRVERGSYVRYNFMNDGFFLKVTALTQKPDVYIPETLEPGQ |
| | PVTVICVFNWAFEECPPPSFSWTGAALSSQGTKPTTSHFSVLSFTPRPQDHN |
| | TDLTCHVDFSRKGVSAQRTVRLRVAYAPRDLVISISRDNTPALEPQPQGNV |
| | PYLEAQKGQFLRLLCAADSQPPATLSWVLQNRVLSSSHPWGPRPLGLELPG |
| | VKAGDSGRYTCRAENRLGSQQRALDLSVQYPPENLRVMVSQANRTVLEN |
| | LGNGTSLPVLEGQSLCLVCVTHSSPPARLSWTQRGQVLSPSQPSDPGVLELP |
| | RVQVEHEGEFTCHARHPLGSQHVSLSLSVHYSPKLLGPSCSWEAEGLHCSC |
| | SSQASPAPSLRWWLGEELLEGNSSQDSFEVTPSSAGPWANSSLSLHGGLSS |
| | GLRLRCEAWNVHGAQSGSILQLPDKKGLIST |
| SEQ ID NO: 757 | DIQMTQSPSSLSASVGDRVTITCKASAAVGTYVAWYQQKPGKAPKLLIYSA |
| a- | SYRKRGVPSRFSGSGSGTDFTLTISSLQPEDFATYYCHQYYTYPLFTFGQGT |
| CEACAM5/CD3 | KLEIKRTVAAPSVFIFPPSDEQLKSGTASVVCLLNNFYPREAKVQWKVDNA |
| LC1- | LQSGNSQESVTEQDSKDSTYSLSSTLTLSKADYEKHKVYACEVTHQGLSSP |
| SIGLEC10ecd | VTKSFNRGECGGGGSGGGGSGGGGSMDGRFWIRVQESVMVPEGLCISVPC |
| | SFSYPRQDWTGSTPAYGYWFKAVTETTKGAPVATNHQSREVEMSTRGRFQ |
| | LTGDPAKGNCSLVIRDAQMQDESQYFFRVERGSYVRYNFMNDGFFLKVTA |
| | LTQKPDVYIPETLEPGQPVTVICVFNWAFEECPPPSFSWTGAALSSQGTKPT |
| | TSHFSVLSFTPRPQDHNTDLTCHVDFSRKGVSAQRTVRLRVAYAPRDLVISI |
| | SRDNTPALEPQPQGNVPYLEAQKGQFLRLLCAADSQPPATLSWVLQNRVL |
| | SSSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 758 | QAVVTQEPSLTVSPGGTVTLTCGSSTGAVTTSNYANWVQEKPGQAFRGLIG |
| a- | GTNKRAPGTPARFSGSLLGGKAALTLSGAQPEDEAEYYCALWYSNLWVFG |
| CEACAM5/CD3 | GGTKLTVLSSASTKGPSVFPLAPSSKSTSGGTAALGCLVKDYFPEPVTVSW |
| LC2- | NSGALTSGVHTFPAVLQSSGLYSLSSVVTVPSSSLGTQTYICNVNHKPSNTK |
| SIGLEC10ecd | VDKKVEPKSCGGGGSGGGGSGGGGSMDGRFWIRVQESVMVPEGLCISVPC |
| | SFSYPRQDWTGSTPAYGYWFKAVTETTKGAPVATNHQSREVEMSTRGRFQ |
| | LTGDPAKGNCSLVIRDAQMQDESQYFFRVERGSYVRYNFMNDGFFLKVTA |
| | LTQKPDVYIPETLEPGQPVTVICVFNWAFEECPPPSFSWTGAALSSQGTKPT |
| | TSHFSVLSFTPRPQDHNTDLTCHVDFSRKGVSAQRTVRLRVAYAPRDLVISI |
| | SRDNTPALEPQPQGNVPYLEAQKGQFLRLLCAADSQPPATLSWVLQNRVL |
| | SSSHPWGPRPLGLELPGVKAGDSGRYTCRAENRLGSQQRALDLSVQYPPEN |
| | LRVMVSQANRTVLENLGNGTSLPVLEGQSLCLVCVTHSSPPARLSWTQRG |
| | QVLSPSQPSDPGVLELPRVQVEHEGEFTCHARHPLGSQHVSLSLSVHYSPKL |
| | LGPSCSWEAEGLHCSCSSQASPAPSLRWWLGEELLEGNSSQDSFEVTPSSA |
| | GPWANSSLSLHGGLSSGLRLRCEAWNVHGAQSGSILQLPDKKGLIST |
| SEQ ID NO: 759 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC1-SIRPaecd | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | GGGGSGGGGSGGGGSEEELQVIQPDKSVLVAAGETATLRCTATSLIPVGPIQ |
| | WFRGAGPGRELIYNQKEGHFPRVTTVSDLTKRNNMDFSIRIGNITPADAGT |
| | YYCVKFRKGSPDDVEFKSGAGTELSVRAKPS |
| SEQ ID NO: 760 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC2-SIRPaecd | LVKDYFPEPVTVSWNSGALTSGVHTFPAVLQSSGLYSLSSVVTVPSSSLGTQ |
| | TYICNVNHKPSNTKVDKKVEPKSCDGGGGSGGGGSEVQLLESGGGLVQPG |
| | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSEEELQVIQPDKSVLVAAGETATLRCTATSLIPVGPIQWFRGAGP |
| | GRELIYNQKEGHFPRVTTVSDLTKRNNMDFSIRIGNITPADAGTYYCVKFR |
| | KGSPDDVEFKSGAGTELSVRAKPS |
| SEQ ID NO: 761 | DIQMTQSPSSLSASVGDRVTITCKASAAVGTYVAWYQQKPGKAPKLLIYSA |
| a- | SYRKRGVPSRFSGSGSGTDFTLTISSLQPEDFATYYCHQYYTYPLFTFGQGT |
| CEACAM5/CD3 | KLEIKRTVAAPSVFIFPPSDEQLKSGTASVVCLLNNFYPREAKVQWKVDNA |
| LC1-SIRPaecd | LQSGNSQESVTEQDSKDSTYSLSSTLTLSKADYEKHKVYACEVTHQGLSSP |
| | VTKSFNRGECGGGGSGGGGSGGGGSEEELQVIQPDKSVLVAAGETATLRC |
| | TATSLIPVGPIQWFRGAGPGRELIYNQKEGHFPRVTTVSDLTKRNNMDFSIR |
| | IGNITPADAGTYYCVKFRKGSPDDVEFKSGAGTELSVRAKPS |
| SEQ ID NO: 762 | QAVVTQEPSLTVSPGGTVTLTCGSSTGAVTTSNYANWVQEKPGQAFRGLIG |
| a- | GTNKRAPGTPARFSGSLLGGKAALTLSGAQPEDEAEYYCALWYSNLWVFG |
| CEACAM5/CD3 | GGTKLTVLSSASTKGPSVFPLAPSSKSTSGGTAALGCLVKDYFPEPVTVSW |
| LC2-SIRPaecd | NSGALTSGVHTFPAVLQSSGLYSLSSVVTVPSSSLGTQTYICNVNHKPSNTK |
| | VDKKVEPKSCGGGGSGGGGSGGGGSEEELQVIQPDKSVLVAAGETATLRC |
| | TATSLIPVGPIQWFRGAGPGRELIYNQKEGHFPRVTTVSDLTKRNNMDFSIR |
| | IGNITPADAGTYYCVKFRKGSPDDVEFKSGAGTELSVRAKPS |
| SEQ ID NO: 763 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC1-TGFbRecd | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | GGGGSGGGGSGGGGSTIPPHVQKSVNNDMIVTDNNGAVKFPQLCKFCDVR |
| | FSTCDNQKSCMSNCSITSICEKPQEVCVAVWRKNDENITLETVCHDPKLPY |
| | HDFILEDAASPKCIMKEKKKPGETFFMCSCSSDECNDNIIFSEEYNTSNPD |
| SEQ ID NO: 764 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC2-TGFbRecd | LVKDYFPEPVTVSWNSGALTSGVHTFPAVLQSSGLYSLSSVVTVPSSSLGTQ |
| | TYICNVNHKPSNTKVDKKVEPKSCDGGGGSGGGGSEVQLLESGGGLVQPG |
| | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSTIPPHVQKSVNNDMIVTDNNGAVKFPQLCKFCDVRFSTCDNQ |
| | KSCMSNCSITSICEKPQEVCVAVWRKNDENITLETVCHDPKLPYHDFILEDA |
| | ASPKCIMKEKKKPGETFFMCSCSSDECNDNIIFSEEYNTSNPD |
| SEQ ID NO: 765 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC1-TIM3ecd | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | GGGGSGGGGSGGGGSSEVEYRAEVGQNAYLPCFYTPAAPGNLVPVCWGK |
| | GACPVFECGNVVLRTDERDVNYWTSRYWLNGDFRKGDVSLTIENVTLADS |
| | GIYCCRIQIPGIMNDEKFNLKLVIKPAKVTPAPTRQRDFTAAFPRMLTTRGH |
| | GPAETQTLGSLPDINLTQISTLANELRDSRLANDLRDSGATIRIG |
| SEQ ID NO: 766 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC2-TIM3ecd | LVKDYFPEPVTVSWNSGALTSGVHTFPAVLQSSGLYSLSSVVTVPSSSLGTQ |
| | TYICNVNHKPSNTKVDKKVEPKSCDGGGGSGGGGSEVQLLESGGGLVQPG |
| | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSSEVEYRAEVGQNAYLPCFYTPAAPGNLVPVCWGKGACPVFEC |
| | GNVVLRTDERDVNYWTSRYWLNGDFRKGDVSLTIENVTLADSGIYCCRIQI |
| | PGIMNDEKFNLKLVIKPAKVTPAPTRQRDFTAAFPRMLTTRGHGPAETQTL |
| | GSLPDINLTQISTLANELRDSRLANDLRDSGATIRIG |
| SEQ ID NO: 767 | DIQMTQSPSSLSASVGDRVTITCKASAAVGTYVAWYQQKPGKAPKLLIYSA |
| a- | SYRKRGVPSRFSGSGSGTDFTLTISSLQPEDFATYYCHQYYTYPLFTFGQGT |
| CEACAM5/CD3 | KLEIKRTVAAPSVFIFPPSDEQLKSGTASVVCLLNNFYPREAKVQWKVDNA |
| LC1-TIM3ecd | LQSGNSQESVTEQDSKDSTYSLSSTLTLSKADYEKHKVYACEVTHQGLSSP |
| | VTKSFNRGECGGGGSGGGGSGGGGSSEVEYRAEVGQNAYLPCFYTPAAPG |
| | NLVPVCWGKGACPVFECGNVVLRTDERDVNYWTSRYWLNGDFRKGDVS |
| | LTIENVTLADSGIYCCRIQIPGIMNDEKFNLKLVIKPAKVTPAPTRQRDFTAA |
| | FPRMLTTRGHGPAETQTLGSLPDINLTQISTLANELRDSRLANDLRDSGATI |
| | RIG |
| SEQ ID NO: 768 | QAVVTQEPSLTVSPGGTVTLTCGSSTGAVTTSNYANWVQEKPGQAFRGLIG |
| a- | GTNKRAPGTPARFSGSLLGGKAALTLSGAQPEDEAEYYCALWYSNLWVFG |
| CEACAM5/CD3 | GGTKLTVLSSASTKGPSVFPLAPSSKSTSGGTAALGCLVKDYFPEPVTVSW |
| LC2-TIM3ecd | NSGALTSGVHTFPAVLQSSGLYSLSSVVTVPSSSLGTQTYICNVNHKPSNTK |
| | VDKKVEPKSCGGGGSGGGGSGGGGSSEVEYRAEVGQNAYLPCFYTPAAPG |
| | NLVPVCWGKGACPVFECGNVVLRTDERDVNYWTSRYWLNGDFRKGDVS |
| | LTIENVTLADSGIYCCRIQIPGIMNDEKFNLKLVIKPAKVTPAPTRQRDFTAA |
| | FPRMLTTRGHGPAETQTLGSLPDINLTQISTLANELRDSRLANDLRDSGATI |
| | RIG |
| SEQ ID NO: 769 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC1-VEGFRecd | LVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPEAAGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALGAPIEKTISKAKGQPREP |
| | QVCTLPPSRDELTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | GGGGSGGGGSGGGGSSDTGRPFVEMYSEIPEIIHMTEGRELVIPCRVTSPNIT |
| | VTLKKFPLDTLIPDGKRIIWDSRKGFIISNATYKEIGLLTCEATVNGHLYKTN |
| | YLTHRQTNTIIDVVLSPSHGIELSVGEKLVLNCTARTELNVGIDFNWEYPSS |
| | KHQHKKLVNRDLKTQSGSEMKKFLSTLTIDGVTRSDQGLYTCAASSGLMT |
| | KKNSTFVRVHEK |
| SEQ ID NO: 770 | QVQLVQSGAEVKKPGASVKVSCKASGYTFTEFGMNWVRQAPGQGLEWM |
| a- | GWINTKTGEATYVEEFKGRVTFTTDTSTSTAYMELRSLRSDDTAVYYCAR |
| CEACAM5/CD3 | WDFAYYVEAMDYWGQGTTVTVSSASTKGPSVFPLAPSSKSTSGGTAALGC |
| HC2-VEGFRecd | LVKDYFPEPVTVSWNSGALTSGVHTFPAVLQSSGLYSLSSVVTVPSSSLGTQ |
| | TYICNVNHKPSNTKVDKKVEPKSCDGGGGSGGGGSEVQLLESGGGLVQPG |
| | GSLRLSCAASGFTFSTYAMNWVRQAPGKGLEWVSRIRSKYNNYATYYADS |
| | VKGRFTISRDDSKNTLYLQMNSLRAEDTAVYYCVRHGNFGNSYVSWFAY |
| | WGQGTLVTVSSASVAAPSVFIFPPSDEQLKSGTASVVCLLNNFYPREAKVQ |
| | WKVDNALQSGNSQESVTEQDSKDSTYSLSSTLTLSKADYEKHKVYACEVT |
| | HQGLSSPVTKSFNRGECDKTHTCPPCPAPEAAGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALGAPIEKTISKAKGQPREPQVYTLPPC |
| | RDELTKNQVSLWCLVKGFYPSDIAVEWESNGQPENNYKTTPPVLDSDGSFF |
| | LYSKLTVDKSRWQQGNVFSCSVMHEALHNHYTQKSLSLSPGKGGGGSGG |
| | GGSGGGGSSDTGRPFVEMYSEIPEIIHMTEGRELVIPCRVTSPNITVTLKKFP |
| | LDTLIPDGKRIIWDSRKGFIISNATYKEIGLLTCEATVNGHLYKTNYLTHRQT |
| | NTIIDVVLSPSHGIELSVGEKLVLNCTARTELNVGIDFNWEYPSSKHQHKKL |
| | VNRDLKTQSGSEMKKFLSTLTIDGVTRSDQGLYTCAASSGLMTKKNSTFVR |
| | VHEK |

Sequences of the Alts of the present invention are found in Table 2.

TABLE 2

| Fusion protein of the invention | Sequence IDs |
|---|---|
| anti-EGFR-BTLAecd-PD1ecd | heavy chain = SEQ ID NO: 220 |
| (cetuximab) | light chain = SEQ ID NO: 221 |
| anti-EGFR-BTLAecd-SIGLEC10ecd | heavy chain = SEQ ID NO: 220 |
| (cetuximab) | light chain = SEQ ID NO: 222 |
| anti-EGFR-BTLAecd-SIRPaecd | heavy chain = SEQ ID NO: 220 |
| (cetuximab) | light chain = SEQ ID NO: 223 |
| anti-EGFR-BTLAecd-TIM3ecd | heavy chain = SEQ ID NO: 220 |
| (cetuximab) | light chain = SEQ ID NO: 224 |
| anti-EGFR-BTLAecd | heavy chain = SEQ ID NO: 220 |
| (cetuximab) | light chain = SEQ ID NO: 43 |
| anti-EGFR-PD1ecd-BTLAecd | heavy chain = SEQ ID NO: 225 |
| (cetuximab) | light chain = SEQ ID NO: 226 |
| anti-EGFR-PD1ecd-SIGLEC10ecd | heavy chain = SEQ ID NO: 225 |
| (cetuximab) | light chain = SEQ ID NO: 222 |
| anti-EGFR-PD1ecd-SIRPaecd | heavy chain = SEQ ID NO: 225 |
| (cetuximab) | light chain = SEQ ID NO: 223 |
| anti-EGFR-PD1ecd-TIM3ecd | heavy chain = SEQ ID NO: 225 |
| (cetuximab) | light chain = SEQ ID NO: 224 |
| anti-EGFR-PD1ecd | heavy chain = SEQ ID NO: 225 |
| (cetuximab) | light chain = SEQ ID NO: 43 |
| anti-EGFR-SIGLEC10ecd-BTLAecd | heavy chain = SEQ ID NO: 227 |
| (cetuximab) | light chain = SEQ ID NO: 226 |
| anti-EGFR-SIGLEC10ecd-PD1ecd | heavy chain = SEQ ID NO: 227 |
| (cetuximab) | light chain = SEQ ID NO: 221 |
| anti-EGFR-SIGLEC10ecd-SIRPaecd | heavy chain = SEQ ID NO: 227 |
| (cetuximab) | light chain = SEQ ID NO: 223 |
| anti-EGFR-SIGLEC10ecd-TIM3ecd | heavy chain = SEQ ID NO: 227 |
| (cetuximab) | light chain = SEQ ID NO: 224 |
| anti-EGFR-SIGLEC10ecd | heavy chain = SEQ ID NO: 227 |
| (cetuximab) | light chain = SEQ ID NO: 43 |
| anti-EGFR-SIRPaecd-BTLAecd | heavy chain = SEQ ID NO: 228 |
| (cetuximab) | light chain = SEQ ID NO: 226 |
| anti-EGFR-SIRPaecd-PD1ecd | heavy chain = SEQ ID NO: 228 |
| (cetuximab) | light chain = SEQ ID NO: 221 |
| anti-EGFR-SIRPaecd-SIGLEC10ecd | heavy chain = SEQ ID NO: 228 |
| (cetuximab) | light chain = SEQ ID NO: 222 |
| anti-EGFR-SIRPaecd-TIM3ecd | heavy chain = SEQ ID NO: 228 |
| (cetuximab) | light chain = SEQ ID NO: 224 |
| anti-EGFR-SIRPaecd | heavy chain = SEQ ID NO: 228 |
| (cetuximab) | light chain = SEQ ID NO: 43 |
| anti-EGFR-TGFbRecd-BTLAecd | heavy chain = SEQ ID NO: 229 |
| (cetuximab) | light chain = SEQ ID NO: 226 |
| anti-EGFR-TGFbRecd-PD1ecd | heavy chain = SEQ ID NO: 229 |
| (cetuximab) | light chain = SEQ ID NO: 221 |
| anti-EGFR-TGFbRecd-SIGLEC10ecd | heavy chain = SEQ ID NO: 229 |
| (cetuximab) | light chain = SEQ ID NO: 222 |
| anti-EGFR-TGFbRecd-SIRPaecd | heavy chain = SEQ ID NO: 229 |
| (cetuximab) | light chain = SEQ ID NO: 223 |
| anti-EGFR-TGFbRecd-TIM3ecd | heavy chain = SEQ ID NO: 229 |
| (cetuximab) | light chain = SEQ ID NO: 224 |
| anti-EGFR-TGFbRecd | heavy chain = SEQ ID NO: 229 |
| (cetuximab) | light chain = SEQ ID NO: 43 |
| anti-EGFR-TIM3ecd-BTLAecd | heavy chain = SEQ ID NO: 230 |
| (cetuximab) | light chain = SEQ ID NO: 226 |
| anti-EGFR-TIM3ecd-PD1ecd | heavy chain = SEQ ID NO: 230 |
| (cetuximab) | light chain = SEQ ID NO: 221 |
| anti-EGFR-TIM3ecd-SIGLEC10ecd | heavy chain = SEQ ID NO: 230 |
| (cetuximab) | light chain = SEQ ID NO: 222 |
| anti-EGFR-TIM3ecd-SIRPaecd | heavy chain = SEQ ID NO: 230 |
| (cetuximab) | light chain = SEQ ID NO: 223 |
| anti-EGFR-TIM3ecd | heavy chain = SEQ ID NO: 230 |
| (cetuximab) | light chain = SEQ ID NO: 43 |
| anti-EGFR-VEGFRecd-BTLAecd | heavy chain = SEQ ID NO: 231 |
| (cetuximab) | light chain = SEQ ID NO: 226 |
| anti-EGFR-VEGFRecd-PD1ecd | heavy chain = SEQ ID NO: 231 |
| (cetuximab) | light chain = SEQ ID NO: 221 |
| anti-EGFR-VEGFRecd-SIGLEC10ecd | heavy chain = SEQ ID NO: 231 |
| (cetuximab) | light chain = SEQ ID NO: 222 |
| anti-EGFR-VEGFRecd-SIRPaecd | heavy chain = SEQ ID NO: 231 |
| (cetuximab) | light chain = SEQ ID NO: 223 |
| anti-EGFR-VEGFRecd-TIM3ecd | heavy chain = SEQ ID NO: 231 |
| (cetuximab) | light chain = SEQ ID NO: 224 |
| anti-EGFR-VEGFRecd | heavy chain = SEQ ID NO: 231 |
| (cetuximab) | light chain = SEQ ID NO: 43 |
| anti-EGFRvIII-BTLAecd-PD1ecd | heavy chain = SEQ ID NO: 232 |
| (depatuxizumab) | light chain = SEQ ID NO: 233 |
| anti-EGFRvIII-BTLAecd-SIGLEC10ecd | heavy chain = SEQ ID NO: 232 |
| (depatuxizumab) | light chain = SEQ ID NO: 234 |
| anti-EGFRvIII-BTLAecd-SIRPaecd | heavy chain = SEQ ID NO: 232 |
| (depatuxizumab) | light chain = SEQ ID NO: 235 |
| anti-EGFRvIII-BTLAecd-TIM3ecd | heavy chain = SEQ ID NO: 232 |
| (depatuxizumab) | light chain = SEQ ID NO: 236 |
| anti-EGFRvIII-BTLAecd | heavy chain = SEQ ID NO: 232 |
| (depatuxizumab) | light chain = SEQ ID NO: 47 |
| anti-EGFRvIII-PD1ecd-BTLAecd | heavy chain = SEQ ID NO: 237 |
| (depatuxizumab) | light chain = SEQ ID NO: 238 |
| anti-EGFRvIII-PD1ecd-SIGLEC10ecd | heavy chain = SEQ ID NO: 237 |
| (depatuxizumab) | light chain = SEQ ID NO: 234 |
| anti-EGFRvIII-PD1ecd-SIRPaecd | heavy chain = SEQ ID NO: 237 |
| (depatuxizumab) | light chain = SEQ ID NO: 235 |
| anti-EGFRvIII-PD1ecd-TIM3ecd | heavy chain = SEQ ID NO: 237 |
| (depatuxizumab) | light chain = SEQ ID NO: 236 |
| anti-EGFRvIII-PD1ecd | heavy chain = SEQ ID NO: 237 |
| (depatuxizumab) | light chain = SEQ ID NO: 47 |
| anti-EGFRvIII-SIGLEC10ecd-BTLAecd | heavy chain = SEQ ID NO: 239 |
| (depatuxizumab) | light chain = SEQ ID NO: 238 |
| anti-EGFRvIII-SIGLEC10ecd-PD1ecd | heavy chain = SEQ ID NO: 239 |
| (depatuxizumab) | light chain = SEQ ID NO: 233 |
| anti-EGFRvIII-SIGLEC10ecd-SIRPaecd | heavy chain = SEQ ID NO: 239 |
| (depatuxizumab) | light chain = SEQ ID NO: 235 |
| anti-EGFRvIII-SIGLEC10ecd-TIM3ecd | heavy chain = SEQ ID NO: 239 |
| (depatuxizumab) | light chain = SEQ ID NO: 236 |
| anti-EGFRvIII-SIGLEC10ecd | heavy chain = SEQ ID NO: 239 |
| (depatuxizumab) | light chain = SEQ ID NO: 47 |
| anti-EGFRvIII-SIRPaecd-BTLAecd | heavy chain = SEQ ID NO: 240 |
| (depatuxizumab) | light chain = SEQ ID NO: 238 |
| anti-EGFRvIII-SIRPaecd-PD1ecd | heavy chain = SEQ ID NO: 240 |
| (depatuxizumab) | light chain = SEQ ID NO: 233 |
| anti-EGFRvIII-SIRPaecd-SIGLEC10ecd | heavy chain = SEQ ID NO: 240 |
| (depatuxizumab) | light chain = SEQ ID NO: 234 |
| anti-EGFRvIII-SIRPaecd-TIM3ecd | heavy chain = SEQ ID NO: 240 |
| (depatuxizumab) | light chain = SEQ ID NO: 236 |
| anti-EGFRvIII-SIRPaecd | heavy chain = SEQ ID NO: 240 |
| (depatuxizumab) | light chain = SEQ ID NO: 47 |
| anti-EGFRvIII-TGFbRecd-BTLAecd | heavy chain = SEQ ID NO: 241 |
| (depatuxizumab) | light chain = SEQ ID NO: 238 |
| anti-EGFRvIII-TGFbRecd-PD1ecd | heavy chain = SEQ ID NO: 241 |
| (depatuxizumab) | light chain = SEQ ID NO: 233 |
| anti-EGFRvIII-TGFbRecd-SIGLEC10ecd | heavy chain = SEQ ID NO: 241 |
| (depatuxizumab) | light chain = SEQ ID NO: 234 |
| anti-EGFRvIII-TGFbRecd-SIRPaecd | heavy chain = SEQ ID NO: 241 |
| (depatuxizumab) | light chain = SEQ ID NO: 235 |
| anti-EGFRvIII-TGFbRecd-TIM3ecd | heavy chain = SEQ ID NO: 241 |
| (depatuxizumab) | light chain = SEQ ID NO: 236 |
| anti-EGFRvIII-TGFbRecd | heavy chain = SEQ ID NO: 241 |
| (depatuxizumab) | light chain = SEQ ID NO: 47 |
| anti-EGFRvIII-TIM3ecd-BTLAecd | heavy chain = SEQ ID NO: 242 |
| (depatuxizumab) | light chain = SEQ ID NO: 238 |
| anti-EGFRvIII-TIM3ecd-PD1ecd | heavy chain = SEQ ID NO: 242 |
| (depatuxizumab) | light chain = SEQ ID NO: 233 |
| anti-EGFRvIII-TIM3ecd-SIGLEC10ecd | heavy chain = SEQ ID NO: 242 |
| (depatuxizumab) | light chain = SEQ ID NO: 234 |
| anti-EGFRvIII-TIM3ecd-SIRPaecd | heavy chain = SEQ ID NO: 242 |
| (depatuxizumab) | light chain = SEQ ID NO: 235 |
| anti-EGFRvIII-TIM3ecd | heavy chain = SEQ ID NO: 242 |
| (depatuxizumab) | light chain = SEQ ID NO: 47 |
| anti-EGFRvIII-VEGFRecd-BTLAecd | heavy chain = SEQ ID NO: 243 |
| (depatuxizumab) | light chain = SEQ ID NO: 238 |
| anti-EGFRvIII-VEGFRecd-PD1ecd | heavy chain = SEQ ID NO: 243 |
| (depatuxizumab) | light chain = SEQ ID NO: 233 |
| anti-EGFRvIII-VEGFRecd-SIGLEC10ecd | heavy chain = SEQ ID NO: 243 |
| (depatuxizumab) | light chain = SEQ ID NO: 234 |
| anti-EGFRvIII-VEGFRecd-SIRPaecd | heavy chain = SEQ ID NO: 243 |
| (depatuxizumab) | light chain = SEQ ID NO: 235 |
| anti-EGFRvIII-VEGFRecd-TIM3ecd | heavy chain = SEQ ID NO: 243 |
| (depatuxizumab) | light chain = SEQ ID NO: 236 |
| anti-EGFRvIII-VEGFRecd | heavy chain = SEQ ID NO: 243 |
| (depatuxizumab) | light chain = SEQ ID NO: 47 |
| anti-HER2-BTLAecd-PD1ecd | heavy chain = SEQ ID NO: 244 |
| (trastuzumab) | light chain = SEQ ID NO: 245 |
| anti-HER2-BTLAecd-SIGLEC10ecd | heavy chain = SEQ ID NO: 244 |
| (trastuzumab) | light chain = SEQ ID NO: 246 |
| anti-HER2-BTLAecd-SIRPaecd | heavy chain = SEQ ID NO: 244 |
| (trastuzumab) | light chain = SEQ ID NO: 247 |
| anti-HER2-BTLAecd-TIM3ecd | heavy chain = SEQ ID NO: 244 |
| (trastuzumab) | light chain = SEQ ID NO: 248 |
| anti-HER2-BTLAecd | heavy chain = SEQ ID NO: 244 |
| (trastuzumab) | light chain = SEQ ID NO: 55 |
| anti-HER2-PD1ecd-BTLAecd | heavy chain = SEQ ID NO: 249 |
| (trastuzumab) | light chain = SEQ ID NO: 250 |
| anti-HER2-PD1ecd-SIGLEC10ecd | heavy chain = SEQ ID NO: 249 |
| (trastuzumab) | light chain = SEQ ID NO: 246 |
| anti-HER2-PD1ecd-SIRPaecd | heavy chain = SEQ ID NO: 249 |
| (trastuzumab) | light chain = SEQ ID NO: 247 |
| anti-HER2-PD1ecd-TIM3ecd | heavy chain = SEQ ID NO: 249 |
| (trastuzumab) | light chain = SEQ ID NO: 248 |
| anti-HER2-PD1ecd | heavy chain = SEQ ID NO: 249 |
| (trastuzumab) | light chain = SEQ ID NO: 55 |
| anti-HER2-SIGLEC10ecd-BTLAecd | heavy chain = SEQ ID NO: 251 |
| (trastuzumab) | light chain = SEQ ID NO: 250 |
| anti-HER2-SIGLEC10ecd-PD1ecd | heavy chain = SEQ ID NO: 251 |
| (trastuzumab) | light chain = SEQ ID NO: 245 |
| anti-HER2-SIGLEC10ecd-SIRPaecd | heavy chain = SEQ ID NO: 251 |
| (trastuzumab) | light chain = SEQ ID NO: 247 |
| anti-HER2-SIGLEC10ecd-TIM3ecd | heavy chain = SEQ ID NO: 251 |
| (trastuzumab) | light chain = SEQ ID NO: 248 |
| anti-HER2-SIGLEC10ecd | heavy chain = SEQ ID NO: 251 |
| (trastuzumab) | light chain = SEQ ID NO: 55 |
| anti-HER2-SIRPaecd-BTLAecd | heavy chain = SEQ ID NO: 252 |
| (trastuzumab) | light chain = SEQ ID NO: 250 |
| anti-HER2-SIRPaecd-PD1ecd | heavy chain = SEQ ID NO: 252 |
| (trastuzumab) | light chain = SEQ ID NO: 245 |
| anti-HER2-SIRPaecd-SIGLEC10ecd | heavy chain = SEQ ID NO: 252 |
| (trastuzumab) | light chain = SEQ ID NO: 246 |
| anti-HER2-SIRPaecd-TIM3ecd | heavy chain = SEQ ID NO: 252 |
| (trastuzumab) | light chain = SEQ ID NO: 248 |
| anti-HER2-SIRPaecd | heavy chain = SEQ ID NO: 252 |
| (trastuzumab) | light chain = SEQ ID NO: 55 |
| anti-HER2-TGFbRecd-BTLAecd | heavy chain = SEQ ID NO: 253 |
| (trastuzumab) | light chain = SEQ ID NO: 250 |
| anti-HER2-TGFbRecd-PD1ecd | heavy chain = SEQ ID NO: 253 |
| (trastuzumab) | light chain = SEQ ID NO: 245 |
| anti-HER2-TGFbRecd-SIGLEC10ecd | heavy chain = SEQ ID NO: 253 |
| (trastuzumab) | light chain = SEQ ID NO: 246 |
| anti-HER2-TGFbRecd-SIRPaecd | heavy chain = SEQ ID NO: 253 |
| (trastuzumab) | light chain = SEQ ID NO: 247 |
| anti-HER2-TGFbRecd-TIM3ecd | heavy chain = SEQ ID NO: 253 |
| (trastuzumab) | light chain = SEQ ID NO: 248 |
| anti-HER2-TGFbRecd | heavy chain = SEQ ID NO: 253 |
| (trastuzumab) | light chain = SEQ ID NO: 55 |
| anti-HER2-TIM3ecd-BTLAecd | heavy chain = SEQ ID NO: 254 |
| (trastuzumab) | light chain = SEQ ID NO: 250 |
| anti-HER2-TIM3ecd-PD1ecd | heavy chain = SEQ ID NO: 254 |
| (trastuzumab) | light chain = SEQ ID NO: 245 |
| anti-HER2-TIM3ecd-SIGLEC10ecd | heavy chain = SEQ ID NO: 254 |
| (trastuzumab) | light chain = SEQ ID NO: 246 |
| anti-HER2-TIM3ecd-SIRPaecd | heavy chain = SEQ ID NO: 254 |
| (trastuzumab) | light chain = SEQ ID NO: 247 |
| anti-HER2-TIM3ecd | heavy chain = SEQ ID NO: 254 |
| (trastuzumab) | light chain = SEQ ID NO: 55 |
| anti-HER2-VEGFRecd-BTLAecd | heavy chain = SEQ ID NO: 255 |
| (trastuzumab) | light chain = SEQ ID NO: 250 |
| anti-HER2-VEGFRecd-PD1ecd | heavy chain = SEQ ID NO: 255 |
| (trastuzumab) | light chain = SEQ ID NO: 245 |
| anti-HER2-VEGFRecd-SIGLEC10ecd | heavy chain = SEQ ID NO: 255 |
| (trastuzumab) | light chain = SEQ ID NO: 246 |
| anti-HER2-VEGFRecd-SIRPaecd | heavy chain = SEQ ID NO: 255 |
| (trastuzumab) | light chain = SEQ ID NO: 247 |
| anti-HER2-VEGFRecd-TIM3ecd | heavy chain = SEQ ID NO: 255 |
| (trastuzumab) | light chain = SEQ ID NO: 248 |
| anti-HER2-VEGFRecd | heavy chain = SEQ ID NO: 255 |
| (trastuzumab) | light chain = SEQ ID NO: 55 |
| anti-nectin4-BTLAecd-PD1ecd | heavy chain = SEQ ID NO: 256 |
| (enfortumab) | light chain = SEQ ID NO: 257 |
| anti-nectin4-BTLAecd-SIGLEC10ecd | heavy chain = SEQ ID NO: 256 |
| (enfortumab) | light chain = SEQ ID NO: 258 |
| anti-nectin4-BTLAecd-SIRPaecd | heavy chain = SEQ ID NO: 256 |
| (enfortumab) | light chain = SEQ ID NO: 259 |
| anti-nectin4-BTLAecd-TIM3ecd | heavy chain = SEQ ID NO: 256 |
| (enfortumab) | light chain = SEQ ID NO: 260 |
| anti-nectin4-BTLAecd | heavy chain = SEQ ID NO: 256 |
| (enfortumab) | light chain = SEQ ID NO: 160 |
| anti-nectin4-PD1ecd-BTLAecd | heavy chain = SEQ ID NO: 261 |
| (enfortumab) | light chain = SEQ ID NO: 262 |
| anti-nectin4-PD1ecd-SIGLEC10ecd | heavy chain = SEQ ID NO: 261 |
| (enfortumab) | light chain = SEQ ID NO: 258 |
| anti-nectin4-PD1ecd-SIRPaecd | heavy chain = SEQ ID NO: 261 |
| (enfortumab) | light chain = SEQ ID NO: 259 |
| anti-nectin4-PD1ecd-TIM3ecd | heavy chain = SEQ ID NO: 261 |
| (enfortumab) | light chain = SEQ ID NO: 260 |
| anti-nectin4-PD1ecd | heavy chain = SEQ ID NO: 261 |
| (enfortumab) | light chain = SEQ ID NO: 160 |
| anti-nectin4-SIGLEC10ecd-BTLAecd | heavy chain = SEQ ID NO: 263 |
| (enfortumab) | light chain = SEQ ID NO: 262 |
| anti-nectin4-SIGLEC10ecd-PD1ecd | heavy chain = SEQ ID NO: 263 |
| (enfortumab) | light chain = SEQ ID NO: 257 |
| anti-nectin4-SIGLEC10ecd-SIRPaecd | heavy chain = SEQ ID NO: 263 |
| (enfortumab) | light chain = SEQ ID NO: 259 |
| anti-nectin4-SIGLEC10ecd-TIM3ecd | heavy chain = SEQ ID NO: 263 |
| (enfortumab) | light chain = SEQ ID NO: 260 |
| anti-nectin4-SIGLEC10ecd | heavy chain = SEQ ID NO: 263 |
| (enfortumab) | light chain = SEQ ID NO: 160 |
| anti-nectin4-SIRPaecd-BTLAecd | heavy chain = SEQ ID NO: 264 |
| (enfortumab) | light chain = SEQ ID NO: 262 |
| anti-nectin4-SIRPaecd-PD1ecd | heavy chain = SEQ ID NO: 264 |
| (enfortumab) | light chain = SEQ ID NO: 257 |
| anti-nectin4-SIRPaecd-SIGLEC10ecd | heavy chain = SEQ ID NO: 264 |
| (enfortumab) | light chain = SEQ ID NO: 258 |
| anti-nectin4-SIRPaecd-TIM3ecd | heavy chain = SEQ ID NO: 264 |
| (enfortumab) | light chain = SEQ ID NO: 260 |
| anti-nectin4-SIRPaecd | heavy chain = SEQ ID NO: 264 |
| (enfortumab) | light chain = SEQ ID NO: 160 |
| anti-nectin4-TGFbRecd-BTLAecd | heavy chain = SEQ ID NO: 265 |
| (enfortumab) | light chain = SEQ ID NO: 262 |
| anti-nectin4-TGFbRecd-PD1ecd | heavy chain = SEQ ID NO: 265 |
| (enfortumab) | light chain = SEQ ID NO: 257 |
| anti-nectin4-TGFbRecd-SIGLEC10ecd | heavy chain = SEQ ID NO: 265 |
| (enfortumab) | light chain = SEQ ID NO: 258 |
| anti-nectin4-TGFbRecd-SIRPaecd | heavy chain = SEQ ID NO: 265 |
| (enfortumab) | light chain = SEQ ID NO: 259 |
| anti-nectin4-TGFbRecd-TIM3ecd | heavy chain = SEQ ID NO: 265 |
| (enfortumab) | light chain = SEQ ID NO: 260 |
| anti-nectin4-TGFbRecd | heavy chain = SEQ ID NO: 265 |
| (enfortumab) | light chain = SEQ ID NO: 160 |
| anti-nectin4-TIM3ecd-BTLAecd | heavy chain = SEQ ID NO: 266 |
| (enfortumab) | light chain = SEQ ID NO: 262 |
| anti-nectin4-TIM3ecd-PD1ecd | heavy chain = SEQ ID NO: 266 |
| (enfortumab) | light chain = SEQ ID NO: 257 |
| anti-nectin4-TIM3ecd-SIGLEC10ecd | heavy chain = SEQ ID NO: 266 |
| (enfortumab) | light chain = SEQ ID NO: 258 |
| anti-nectin4-TIM3ecd-SIRPaecd | heavy chain = SEQ ID NO: 266 |
| (enfortumab) | light chain = SEQ ID NO: 259 |
| anti-nectin4-TIM3ecd | heavy chain = SEQ ID NO: 266 |
| (enfortumab) | light chain = SEQ ID NO: 160 |
| anti-nectin4-VEGFRecd-BTLAecd | heavy chain = SEQ ID NO: 267 |
| (enfortumab) | light chain = SEQ ID NO: 262 |
| anti-nectin4-VEGFRecd-PD1ecd | heavy chain = SEQ ID NO: 267 |
| (enfortumab) | light chain = SEQ ID NO: 257 |
| anti-nectin4-VEGFRecd-SIGLEC10ecd | heavy chain = SEQ ID NO: 267 |
| (enfortumab) | light chain = SEQ ID NO: 258 |
| anti-nectin4-VEGFRecd-SIRPaecd | heavy chain = SEQ ID NO: 267 |
| (enfortumab) | light chain = SEQ ID NO: 259 |
| anti-nectin4-VEGFRecd-TIM3ecd | heavy chain = SEQ ID NO: 267 |
| (enfortumab) | light chain = SEQ ID NO: 260 |
| anti-nectin4-VEGFRecd | heavy chain = SEQ ID NO: 267 |
| (enfortumab) | light chain = SEQ ID NO: 160 |
| anti-uPAR-BTLAecd | heavy chain = SEQ ID NO: 268 |
| (ab1) | light chain = SEQ ID NO: 162 |
| anti-uPAR-PD1ecd | heavy chain = SEQ ID NO: 269 |
| (ab1) | light chain = SEQ ID NO: 162 |
| anti-uPAR-SIGLEC10ecd | heavy chain = SEQ ID NO: 270 |
| (ab1) | light chain = SEQ ID NO: 162 |
| anti-uPAR-SIRPaecd | heavy chain = SEQ ID NO: 271 |
| (ab1) | light chain = SEQ ID NO: 162 |
| anti-uPAR-TGFbRecd | heavy chain = SEQ ID NO: 272 |
| (ab1) | light chain = SEQ ID NO: 162 |
| anti-uPAR-TIM3ecd | heavy chain = SEQ ID NO: 273 |
| (ab1) | light chain = SEQ ID NO: 162 |
| anti-uPAR-VEGFRecd | heavy chain = SEQ ID NO: 274 |
| (ab1) | light chain = SEQ ID NO: 162 |
| anti-PSMA-BTLAecd | heavy chain = SEQ ID NO: 275 |
| (ab1) | light chain = SEQ ID NO: 121 |
| anti-PSMA-PD1ecd | heavy chain = SEQ ID NO: 276 |
| (ab1) | light chain = SEQ ID NO: 121 |
| anti-PSMA-SIGLEC10ecd | heavy chain = SEQ ID NO: 277 |
| (ab1) | light chain = SEQ ID NO: 121 |
| anti-PSMA-SIRPaecd | heavy chain = SEQ ID NO: 278 |
| (ab1) | light chain = SEQ ID NO: 121 |
| anti-PSMA-TGFbRecd | heavy chain = SEQ ID NO: 279 |
| (ab1) | light chain = SEQ ID NO: 121 |
| anti-PSMA-TIM3ecd | heavy chain = SEQ ID NO: 280 |
| (ab1) | light chain = SEQ ID NO: 121 |
| anti-PSMA-VEGFRecd | heavy chain = SEQ ID NO: 281 |
| (ab1) | light chain = SEQ ID NO: 121 |
| anti-CEACAM5-BTLAecd | heavy chain = SEQ ID NO: 282 |
| (labetuzumab) | light chain = SEQ ID NO: 26 |
| anti-CEACAM5-PD1ecd | heavy chain = SEQ ID NO: 283 |
| (labetuzumab) | light chain = SEQ ID NO: 26 |
| anti-CEACAM5-SIGLEC10ecd | heavy chain = SEQ ID NO: 284 |
| (labetuzumab) | light chain = SEQ ID NO: 26 |
| anti-CEACAM5-SIRPaecd | heavy chain = SEQ ID NO: 285 |
| (labetuzumab) | light chain = SEQ ID NO: 26 |
| anti-CEACAM5-TGFbRecd | heavy chain = SEQ ID NO: 286 |
| (labetuzumab) | light chain = SEQ ID NO: 26 |
| anti-CEACAM5-TIM3ecd | heavy chain = SEQ ID NO: 287 |
| (labetuzumab) | light chain = SEQ ID NO: 26 |
| anti-CEACAM5-VEGFRecd | heavy chain = SEQ ID NO: 288 |
| (labetuzumab) | light chain = SEQ ID NO: 26 |
| anti-CD38-BTLAecd-PD1ecd | heavy chain = SEQ ID NO: 289 |
| (daratumumab) | light chain = SEQ ID NO: 290 |
| anti-CD38-BTLAecd-SIGLEC10ecd | heavy chain = SEQ ID NO: 289 |
| (daratumumab) | light chain = SEQ ID NO: 291 |
| anti-CD38-BTLAecd-SIRPaecd | heavy chain = SEQ ID NO: 289 |
| (daratumumab) | light chain = SEQ ID NO: 292 |
| anti-CD38-BTLAecd-TIM3ecd | heavy chain = SEQ ID NO: 289 |
| (daratumumab) | light chain = SEQ ID NO: 293 |
| anti-CD38-BTLAecd | heavy chain = SEQ ID NO: 289 |
| (daratumumab) | light chain = SEQ ID NO: 16 |
| anti-CD38-PD1ecd-BTLAecd | heavy chain = SEQ ID NO: 294 |
| (daratumumab) | light chain = SEQ ID NO: 295 |
| anti-CD38-PD1ecd-SIGLEC10ecd | heavy chain = SEQ ID NO: 294 |
| (daratumumab) | light chain = SEQ ID NO: 291 |
| anti-CD38-PD1ecd-SIRPaecd | heavy chain = SEQ ID NO: 294 |
| (daratumumab) | light chain = SEQ ID NO: 292 |
| anti-CD38-PD1ecd-TIM3ecd | heavy chain = SEQ ID NO: 294 |
| (daratumumab) | light chain = SEQ ID NO: 293 |
| anti-CD38-PD1ecd | heavy chain = SEQ ID NO: 294 |
| (daratumumab) | light chain = SEQ ID NO: 16 |
| anti-CD38-SIGLEC10ecd-BTLAecd | heavy chain = SEQ ID NO: 296 |
| (daratumumab) | light chain = SEQ ID NO: 295 |
| anti-CD38-SIGLEC10ecd-PD1ecd | heavy chain = SEQ ID NO: 296 |
| (daratumumab) | light chain = SEQ ID NO: 290 |
| anti-CD38-SIGLEC10ecd-SIRPaecd | heavy chain = SEQ ID NO: 296 |
| (daratumumab) | light chain = SEQ ID NO: 292 |
| anti-CD38-SIGLEC10ecd-TIM3ecd | heavy chain = SEQ ID NO: 296 |
| (daratumumab) | light chain = SEQ ID NO: 293 |
| anti-CD38-SIGLEC10ecd | heavy chain = SEQ ID NO: 296 |
| (daratumumab) | light chain = SEQ ID NO: 16 |
| anti-CD38-SIRPaecd-BTLAecd | heavy chain = SEQ ID NO: 297 |
| (daratumumab) | light chain = SEQ ID NO: 295 |
| anti-CD38-SIRPaecd-PD1ecd | heavy chain = SEQ ID NO: 297 |
| (daratumumab) | light chain = SEQ ID NO: 290 |
| anti-CD38-SIRPaecd-SIGLEC10ecd | heavy chain = SEQ ID NO: 297 |
| (daratumumab) | light chain = SEQ ID NO: 291 |
| anti-CD38-SIRPaecd-TIM3ecd | heavy chain = SEQ ID NO: 297 |
| (daratumumab) | light chain = SEQ ID NO: 293 |
| anti-CD38-SIRPaecd | heavy chain = SEQ ID NO: 297 |
| (daratumumab) | light chain = SEQ ID NO: 16 |
| anti-CD38-TGFbRecd-BTLAecd | heavy chain = SEQ ID NO: 298 |
| (daratumumab) | light chain = SEQ ID NO: 295 |
| anti-CD38-TGFbRecd-PD1ecd | heavy chain = SEQ ID NO: 298 |
| (daratumumab) | light chain = SEQ ID NO: 290 |
| anti-CD38-TGFbRecd-SIGLEC10ecd | heavy chain = SEQ ID NO: 298 |
| (daratumumab) | light chain = SEQ ID NO: 291 |
| anti-CD38-TGFbRecd-SIRPaecd | heavy chain = SEQ ID NO: 298 |
| (daratumumab) | light chain = SEQ ID NO: 292 |
| anti-CD38-TGFbRecd-TIM3ecd | heavy chain = SEQ ID NO: 298 |
| (daratumumab) | light chain = SEQ ID NO: 293 |
| anti-CD38-TGFbRecd | heavy chain = SEQ ID NO: 298 |
| (daratumumab) | light chain = SEQ ID NO: 16 |
| anti-CD38-TIM3ecd-BTLAecd | heavy chain = SEQ ID NO: 299 |
| (daratumumab) | light chain = SEQ ID NO: 295 |
| anti-CD38-TIM3ecd-PD1ecd | heavy chain = SEQ ID NO: 299 |
| (daratumumab) | light chain = SEQ ID NO: 290 |
| anti-CD38-TIM3ecd-SIGLEC10ecd | heavy chain = SEQ ID NO: 299 |
| (daratumumab) | light chain = SEQ ID NO: 291 |
| anti-CD38-TIM3ecd-SIRPaecd | heavy chain = SEQ ID NO: 299 |
| (daratumumab) | light chain = SEQ ID NO: 292 |
| anti-CD38-TIM3ecd | heavy chain = SEQ ID NO: 299 |
| (daratumumab) | light chain = SEQ ID NO: 16 |
| anti-CD38-VEGFRecd-BTLAecd | heavy chain = SEQ ID NO: 300 |
| (daratumumab) | light chain = SEQ ID NO: 295 |
| anti-CD38-VEGFRecd-PD1ecd | heavy chain = SEQ ID NO: 300 |
| (daratumumab) | light chain = SEQ ID NO: 290 |
| anti-CD38-VEGFRecd-SIGLEC10ecd | heavy chain = SEQ ID NO: 300 |
| (daratumumab) | light chain = SEQ ID NO: 291 |
| anti-CD38-VEGFRecd-SIRPaecd | heavy chain = SEQ ID NO: 300 |
| (daratumumab) | light chain = SEQ ID NO: 292 |
| anti-CD38-VEGFRecd-TIM3ecd | heavy chain = SEQ ID NO: 300 |
| (daratumumab) | light chain = SEQ ID NO: 293 |
| anti-CD38-VEGFRecd | heavy chain = SEQ ID NO: 300 |
| (daratumumab) | light chain = SEQ ID NO: 16 |
| anti-SLAMF7-BTLAecd-PD1ecd | heavy chain = SEQ ID NO: 301 |
| (elotuzumab) | light chain = SEQ ID NO: 302 |
| anti-SLAMF7-BTLAecd-SIGLEC10ecd | heavy chain = SEQ ID NO: 301 |
| (elotuzumab) | light chain = SEQ ID NO: 303 |
| anti-SLAMF7-BTLAecd-SIRPaecd | heavy chain = SEQ ID NO: 301 |
| (elotuzumab) | light chain = SEQ ID NO: 304 |
| anti-SLAMF7-BTLAecd-TIM3ecd | heavy chain = SEQ ID NO: 301 |
| (elotuzumab) | light chain = SEQ ID NO: 305 |
| anti-SLAMF7-BTLAecd | heavy chain = SEQ ID NO: 301 |
| (elotuzumab) | light chain = SEQ ID NO: 127 |
| anti-SLAMF7-PD1ecd-BTLAecd | heavy chain = SEQ ID NO: 306 |
| (elotuzumab) | light chain = SEQ ID NO: 307 |
| anti-SLAMF7-PD1ecd-SIGLEC10ecd | heavy chain = SEQ ID NO: 306 |
| (elotuzumab) | light chain = SEQ ID NO: 303 |
| anti-SLAMF7-PD1ecd-SIRPaecd | heavy chain = SEQ ID NO: 306 |
| (elotuzumab) | light chain = SEQ ID NO: 304 |
| anti-SLAMF7-PD1ecd-TIM3ecd | heavy chain = SEQ ID NO: 306 |
| (elotuzumab) | light chain = SEQ ID NO: 305 |
| anti-SLAMF7-PD1ecd | heavy chain = SEQ ID NO: 306 |
| (elotuzumab) | light chain = SEQ ID NO: 127 |
| anti-SLAMF7-SIGLEC10ecd-BTLAecd | heavy chain = SEQ ID NO: 308 |
| (elotuzumab) | light chain = SEQ ID NO: 307 |
| anti-SLAMF7-SIGLEC10ecd-PD1ecd | heavy chain = SEQ ID NO: 308 |
| (elotuzumab) | light chain = SEQ ID NO: 302 |
| anti-SLAMF7-SIGLEC10ecd-SIRPaecd | heavy chain = SEQ ID NO: 308 |
| (elotuzumab) | light chain = SEQ ID NO: 304 |
| anti-SLAMF7-SIGLEC10ecd-TIM3ecd | heavy chain = SEQ ID NO: 308 |
| (elotuzumab) | light chain = SEQ ID NO: 305 |
| anti-SLAMF7-SIGLEC10ecd | heavy chain = SEQ ID NO: 308 |
| (elotuzumab) | light chain = SEQ ID NO: 127 |
| anti-SLAMF7-SIRPaecd-BTLAecd | heavy chain = SEQ ID NO: 309 |
| (elotuzumab) | light chain = SEQ ID NO: 307 |
| anti-SLAMF7-SIRPaecd-PD1ecd | heavy chain = SEQ ID NO: 309 |
| (elotuzumab) | light chain = SEQ ID NO: 302 |
| anti-SLAMF7-SIRPaecd-SIGLEC10ecd | heavy chain = SEQ ID NO: 309 |
| (elotuzumab) | light chain = SEQ ID NO: 303 |
| anti-SLAMF7-SIRPaecd-TIM3ecd | heavy chain = SEQ ID NO: 309 |
| (elotuzumab) | light chain = SEQ ID NO: 305 |
| anti-SLAMF7-SIRPaecd | heavy chain = SEQ ID NO: 309 |
| (elotuzumab) | light chain = SEQ ID NO: 127 |
| anti-SLAMF7-TGFbRecd-BTLAecd | heavy chain = SEQ ID NO: 310 |
| (elotuzumab) | light chain = SEQ ID NO: 307 |
| anti-SLAMF7-TGFbRecd-PD1ecd | heavy chain = SEQ ID NO: 310 |
| (elotuzumab) | light chain = SEQ ID NO: 302 |
| anti-SLAMF7-TGFbRecd-SIGLEC10ecd | heavy chain = SEQ ID NO: 310 |
| (elotuzumab) | light chain = SEQ ID NO: 303 |
| anti-SLAMF7-TGFbRecd-SIRPaecd | heavy chain = SEQ ID NO: 310 |
| (elotuzumab) | light chain = SEQ ID NO: 304 |
| anti-SLAMF7-TGFbRecd-TIM3ecd | heavy chain = SEQ ID NO: 310 |
| (elotuzumab) | light chain = SEQ ID NO: 305 |
| anti-SLAMF7-TGFbRecd | heavy chain = SEQ ID NO: 310 |
| (elotuzumab) | light chain = SEQ ID NO: 127 |
| anti-SLAMF7-TIM3ecd-BTLAecd | heavy chain = SEQ ID NO: 311 |
| (elotuzumab) | light chain = SEQ ID NO: 307 |
| anti-SLAMF7-TIM3ecd-PD1ecd | heavy chain = SEQ ID NO: 311 |
| (elotuzumab) | light chain = SEQ ID NO: 302 |
| anti-SLAMF7-TIM3ecd-SIGLEC10ecd | heavy chain = SEQ ID NO: 311 |
| (elotuzumab) | light chain = SEQ ID NO: 303 |
| anti-SLAMF7-TIM3ecd-SIRPaecd | heavy chain = SEQ ID NO: 311 |
| (elotuzumab) | light chain = SEQ ID NO: 304 |
| anti-SLAMF7-TIM3ecd | heavy chain = SEQ ID NO: 311 |
| (elotuzumab) | light chain = SEQ ID NO: 127 |
| anti-SLAMF7-VEGFRecd-BTLAecd | heavy chain = SEQ ID NO: 312 |
| (elotuzumab) | light chain = SEQ ID NO: 307 |
| anti-SLAMF7-VEGFRecd-PD1ecd | heavy chain = SEQ ID NO: 312 |
| (elotuzumab) | light chain = SEQ ID NO: 302 |
| anti-SLAMF7-VEGFRecd-SIGLEC10ecd | heavy chain = SEQ ID NO: 312 |
| (elotuzumab) | light chain = SEQ ID NO: 303 |
| anti-SLAMF7-VEGFRecd-SIRPaecd | heavy chain = SEQ ID NO: 312 |
| (elotuzumab) | light chain = SEQ ID NO: 304 |
| anti-SLAMF7-VEGFRecd-TIM3ecd | heavy chain = SEQ ID NO: 312 |
| (elotuzumab) | light chain = SEQ ID NO: 305 |
| anti-SLAMF7-VEGFRecd | heavy chain = SEQ ID NO: 312 |
| (elotuzumab) | light chain = SEQ ID NO: 127 |
| anti-IL6R-BTLAecd-PD1ecd | heavy chain = SEQ ID NO: 313 |
| (tocilizumab) | light chain = SEQ ID NO: 314 |
| anti-IL6R-BTLAecd-SIGLEC10ecd | heavy chain = SEQ ID NO: 313 |
| (tocilizumab) | light chain = SEQ ID NO: 315 |
| anti-IL6R-BTLAecd-SIRPaecd | heavy chain = SEQ ID NO: 313 |
| (tocilizumab) | light chain = SEQ ID NO: 316 |
| anti-IL6R-BTLAecd-TIM3ecd | heavy chain = SEQ ID NO: 313 |
| (tocilizumab) | light chain = SEQ ID NO: 317 |
| anti-IL6R-BTLAecd | heavy chain = SEQ ID NO: 313 |
| (tocilizumab) | light chain = SEQ ID NO: 79 |
| anti-IL6R-PD1ecd-BTLAecd | heavy chain = SEQ ID NO: 318 |
| (tocilizumab) | light chain = SEQ ID NO: 319 |
| anti-IL6R-PD1ecd-SIGLEC10ecd | heavy chain = SEQ ID NO: 318 |
| (tocilizumab) | light chain = SEQ ID NO: 315 |
| anti-IL6R-PD1ecd-SIRPaecd | heavy chain = SEQ ID NO: 318 |
| (tocilizumab) | light chain = SEQ ID NO: 316 |
| anti-IL6R-PD1ecd-TIM3ecd | heavy chain = SEQ ID NO: 318 |
| (tocilizumab) | light chain = SEQ ID NO: 317 |
| anti-IL6R-PD1ecd | heavy chain = SEQ ID NO: 318 |
| (tocilizumab) | light chain = SEQ ID NO: 79 |
| anti-IL6R-SIGLEC10ecd-BTLAecd | heavy chain = SEQ ID NO: 320 |
| (tocilizumab) | light chain = SEQ ID NO: 319 |
| anti-IL6R-SIGLEC10ecd-PD1ecd | heavy chain = SEQ ID NO: 320 |
| (tocilizumab) | light chain = SEQ ID NO: 314 |
| anti-IL6R-SIGLEC10ecd-SIRPaecd | heavy chain = SEQ ID NO: 320 |
| (tocilizumab) | light chain = SEQ ID NO: 316 |
| anti-IL6R-SIGLEC10ecd-TIM3ecd | heavy chain = SEQ ID NO: 320 |
| (tocilizumab) | light chain = SEQ ID NO: 317 |
| anti-IL6R-SIGLEC10ecd | heavy chain = SEQ ID NO: 320 |
| (tocilizumab) | light chain = SEQ ID NO: 79 |
| anti-IL6R-SIRPaecd-BTLAecd | heavy chain = SEQ ID NO: 321 |
| (tocilizumab) | light chain = SEQ ID NO: 319 |
| anti-IL6R-SIRPaecd-PD1ecd | heavy chain = SEQ ID NO: 321 |
| (tocilizumab) | light chain = SEQ ID NO: 314 |
| anti-IL6R-SIRPaecd-SIGLEC10ecd | heavy chain = SEQ ID NO: 321 |
| (tocilizumab) | light chain = SEQ ID NO: 315 |
| anti-IL6R-SIRPaecd-TIM3ecd | heavy chain = SEQ ID NO: 321 |
| (tocilizumab) | light chain = SEQ ID NO: 317 |
| anti-IL6R-SIRPaecd | heavy chain = SEQ ID NO: 321 |
| (tocilizumab) | light chain = SEQ ID NO: 79 |
| anti-IL6R-TGFbRecd-BTLAecd | heavy chain = SEQ ID NO: 322 |
| (tocilizumab) | light chain = SEQ ID NO: 319 |
| anti-IL6R-TGFbRecd-PD1ecd | heavy chain = SEQ ID NO: 322 |
| (tocilizumab) | light chain = SEQ ID NO: 314 |
| anti-IL6R-TGFbRecd-SIGLEC10ecd | heavy chain = SEQ ID NO: 322 |
| (tocilizumab) | light chain = SEQ ID NO: 315 |
| anti-IL6R-TGFbRecd-SIRPaecd | heavy chain = SEQ ID NO: 322 |
| (tocilizumab) | light chain = SEQ ID NO: 316 |
| anti-IL6R-TGFbRecd-TIM3ecd | heavy chain = SEQ ID NO: 322 |
| (tocilizumab) | light chain = SEQ ID NO: 317 |
| anti-IL6R-TGFbRecd | heavy chain = SEQ ID NO: 322 |
| (tocilizumab) | light chain = SEQ ID NO: 79 |
| anti-IL6R-TIM3ecd-BTLAecd | heavy chain = SEQ ID NO: 323 |
| (tocilizumab) | light chain = SEQ ID NO: 319 |
| anti-IL6R-TIM3ecd-PD1ecd | heavy chain = SEQ ID NO: 323 |
| (tocilizumab) | light chain = SEQ ID NO: 314 |
| anti-IL6R-TIM3ecd-SIGLEC10ecd | heavy chain = SEQ ID NO: 323 |
| (tocilizumab) | light chain = SEQ ID NO: 315 |
| anti-IL6R-TIM3ecd-SIRPaecd | heavy chain = SEQ ID NO: 323 |
| (tocilizumab) | light chain = SEQ ID NO: 316 |
| anti-IL6R-TIM3ecd | heavy chain = SEQ ID NO: 323 |
| (tocilizumab) | light chain = SEQ ID NO: 79 |
| anti-IL6R-VEGFRecd-BTLAecd | heavy chain = SEQ ID NO: 324 |
| (tocilizumab) | light chain = SEQ ID NO: 319 |
| anti-IL6R-VEGFRecd-PD1ecd | heavy chain = SEQ ID NO: 324 |
| (tocilizumab) | light chain = SEQ ID NO: 314 |
| anti-IL6R-VEGFRecd-SIGLEC10ecd | heavy chain = SEQ ID NO: 324 |
| (tocilizumab) | light chain = SEQ ID NO: 315 |
| anti-IL6R-VEGFRecd-SIRPaecd | heavy chain = SEQ ID NO: 324 |
| (tocilizumab) | light chain = SEQ ID NO: 316 |
| anti-IL6R-VEGFRecd-TIM3ecd | heavy chain = SEQ ID NO: 324 |
| (tocilizumab) | light chain = SEQ ID NO: 317 |
| anti-IL6R-VEGFRecd | heavy chain = SEQ ID NO: 324 |
| (tocilizumab) | light chain = SEQ ID NO: 79 |
| anti-IL17R-BTLAecd-PD1ecd | heavy chain = SEQ ID NO: 325 |
| (brodalumab) | light chain = SEQ ID NO: 326 |
| anti-IL17R-BTLAecd-SIGLEC10ecd | heavy chain = SEQ ID NO: 325 |
| (brodalumab) | light chain = SEQ ID NO: 327 |
| anti-IL17R-BTLAecd-SIRPaecd | heavy chain = SEQ ID NO: 325 |
| (brodalumab) | light chain = SEQ ID NO: 328 |
| anti-IL17R-BTLAecd-TIM3ecd | heavy chain = SEQ ID NO: 325 |
| (brodalumab) | light chain = SEQ ID NO: 329 |
| anti-IL17R-BTLAecd | heavy chain = SEQ ID NO: 325 |
| (brodalumab) | light chain = SEQ ID NO: 63 |
| anti-IL17R-PD1ecd-BTLAecd | heavy chain = SEQ ID NO: 330 |
| (brodalumab) | light chain = SEQ ID NO: 331 |
| anti-IL17R-PD1ecd-SIGLEC10ecd | heavy chain = SEQ ID NO: 330 |
| (brodalumab) | light chain = SEQ ID NO: 327 |
| anti-IL17R-PD1ecd-SIRPaecd | heavy chain = SEQ ID NO: 330 |
| (brodalumab) | light chain = SEQ ID NO: 328 |
| anti-IL17R-PD1ecd-TIM3ecd | heavy chain = SEQ ID NO: 330 |
| (brodalumab) | light chain = SEQ ID NO: 329 |
| anti-IL17R-PD1ecd | heavy chain = SEQ ID NO: 330 |
| (brodalumab) | light chain = SEQ ID NO: 63 |
| anti-IL17R-SIGLEC10cd-BTLAecd | heavy chain = SEQ ID NO: 332 |
| (brodalumab) | light chain = SEQ ID NO: 331 |
| anti-IL17R-SIGLEC10cd-PD1ecd | heavy chain = SEQ ID NO: 332 |
| (brodalumab) | light chain = SEQ ID NO: 326 |
| anti-IL17R-SIGLEC10ecd-SIRPaecd | heavy chain = SEQ ID NO: 332 |
| (brodalumab) | light chain = SEQ ID NO: 328 |
| anti-IL17R-SIGLEC10ecd-TIM3ecd | heavy chain = SEQ ID NO: 332 |
| (brodalumab) | light chain = SEQ ID NO: 329 |
| anti-IL17R-SIGLEC10ecd | heavy chain = SEQ ID NO: 332 |
| (brodalumab) | light chain = SEQ ID NO: 63 |
| anti-IL17R-SIRPaecd-BTLAecd | heavy chain = SEQ ID NO: 333 |
| (brodalumab) | light chain = SEQ ID NO: 331 |
| anti-IL17R-SIRPaecd-PD1ecd | heavy chain = SEQ ID NO: 333 |
| (brodalumab) | light chain = SEQ ID NO: 326 |
| anti-IL17R-SIRPaecd-SIGLEC10ecd | heavy chain = SEQ ID NO: 333 |
| (brodalumab) | light chain = SEQ ID NO: 327 |
| anti-IL17R-SIRPaecd-TIM3ecd | heavy chain = SEQ ID NO: 333 |
| (brodalumab) | light chain = SEQ ID NO: 329 |
| anti-IL17R-SIRPaecd | heavy chain = SEQ ID NO: 333 |
| (brodalumab) | light chain = SEQ ID NO: 63 |
| anti-IL17R-TGFbRecd-BTLAecd | heavy chain = SEQ ID NO: 334 |
| (brodalumab) | light chain = SEQ ID NO: 331 |
| anti-IL17R-TGFbRecd-PD1ecd | heavy chain = SEQ ID NO: 334 |
| (brodalumab) | light chain = SEQ ID NO: 326 |
| anti-IL17R-TGFbRecd-SIGLEC10ecd | heavy chain = SEQ ID NO: 334 |
| (brodalumab) | light chain = SEQ ID NO: 327 |
| anti-IL17R-TGFbRecd-SIRPaecd | heavy chain = SEQ ID NO: 334 |
| (brodalumab) | light chain = SEQ ID NO: 328 |
| anti-IL17R-TGFbRecd-TIM3ecd | heavy chain = SEQ ID NO: 334 |
| (brodalumab) | light chain = SEQ ID NO: 329 |
| anti-IL17R-TGFbRecd | heavy chain = SEQ ID NO: 334 |
| (brodalumab) | light chain = SEQ ID NO: 63 |
| anti-IL17R-TIM3ecd-BTLAecd | heavy chain = SEQ ID NO: 335 |
| (brodalumab) | light chain = SEQ ID NO: 331 |
| anti-IL17R-TIM3ecd-PD1ecd | heavy chain = SEQ ID NO: 335 |
| (brodalumab) | light chain = SEQ ID NO: 326 |
| anti-IL17R-TIM3ecd-SIGLEC10ecd | heavy chain = SEQ ID NO: 335 |
| (brodalumab) | light chain = SEQ ID NO: 327 |
| anti-IL17R-TIM3ecd-SIRPaecd | heavy chain = SEQ ID NO: 335 |
| (brodalumab) | light chain = SEQ ID NO: 328 |
| anti-IL17R-TIM3ecd | heavy chain = SEQ ID NO: 335 |
| (brodalumab) | light chain = SEQ ID NO: 63 |
| anti-IL17R-VEGFRecd-BTLAecd | heavy chain = SEQ ID NO: 336 |
| (brodalumab) | light chain = SEQ ID NO: 331 |
| anti-IL17R-VEGFRecd-PD1ecd | heavy chain = SEQ ID NO: 336 |
| (brodalumab) | light chain = SEQ ID NO: 326 |
| anti-IL17R-VEGFRecd-SIGLEC10ecd | heavy chain = SEQ ID NO: 336 |
| (brodalumab) | light chain = SEQ ID NO: 327 |
| anti-IL17R-VEGFRecd-SIRPaecd | heavy chain = SEQ ID NO: 336 |
| (brodalumab) | light chain = SEQ ID NO: 328 |
| anti-IL17R-VEGFRecd-TIM3ecd | heavy chain = SEQ ID NO: 336 |
| (brodalumab) | light chain = SEQ ID NO: 329 |
| anti-IL17R-VEGFRecd | heavy chain = SEQ ID NO: 336 |
| (brodalumab) | light chain = SEQ ID NO: 63 |
| anti-IL23-BTLAecd-PD1ecd | heavy chain = SEQ ID NO: 337 |
| (risankizumab) | light chain = SEQ ID NO: 338 |
| anti-IL23-BTLAecd-SIGLEC10ecd | heavy chain = SEQ ID NO: 337 |
| (risankizumab) | light chain = SEQ ID NO: 339 |
| anti-IL23-BTLAecd-SIRPaecd | heavy chain = SEQ ID NO: 337 |
| (risankizumab) | light chain = SEQ ID NO: 340 |
| anti-IL23-BTLAecd-TIM3ecd | heavy chain = SEQ ID NO: 337 |
| (risankizumab) | light chain = SEQ ID NO: 341 |
| anti-IL23-BTLAecd | heavy chain = SEQ ID NO: 337 |
| (risankizumab) | light chain = SEQ ID NO: 75 |
| anti-IL23-PD1ecd-BTLAecd | heavy chain = SEQ ID NO: 342 |
| (risankizumab) | light chain = SEQ ID NO: 343 |
| anti-IL23-PD1ecd-SIGLEC10ecd | heavy chain = SEQ ID NO: 342 |
| (risankizumab) | light chain = SEQ ID NO: 339 |
| anti-IL23-PD1ecd-SIRPaecd | heavy chain = SEQ ID NO: 342 |
| (risankizumab) | light chain = SEQ ID NO: 340 |
| anti-IL23-PD1ecd-TIM3ecd | heavy chain = SEQ ID NO: 342 |
| (risankizumab) | light chain = SEQ ID NO: 341 |
| anti-IL23-PD1ecd | heavy chain = SEQ ID NO: 342 |
| (risankizumab) | light chain = SEQ ID NO: 75 |
| anti-IL23-SIGLEC10ecd-BTLAecd | heavy chain = SEQ ID NO: 344 |
| (risankizumab) | light chain = SEQ ID NO: 343 |
| anti-IL23-SIGLEC10ecd-PD1ecd | heavy chain = SEQ ID NO: 344 |
| (risankizumab) | light chain = SEQ ID NO: 338 |
| anti-IL23-SIGLEC10ecd-SIRPaecd | heavy chain = SEQ ID NO: 344 |
| (risankizumab) | light chain = SEQ ID NO: 340 |
| anti-IL23-SIGLEC10ecd-TIM3ecd | heavy chain = SEQ ID NO: 344 |
| (risankizumab) | light chain = SEQ ID NO: 341 |
| anti-IL23-SIGLEC10ecd | heavy chain = SEQ ID NO: 344 |
| (risankizumab) | light chain = SEQ ID NO: 75 |
| anti-IL23-SIRPaecd-BTLAecd | heavy chain = SEQ ID NO: 345 |
| (risankizumab) | light chain = SEQ ID NO: 343 |
| anti-IL23-SIRPaecd-PD1ecd | heavy chain = SEQ ID NO: 345 |
| (risankizumab) | light chain = SEQ ID NO: 338 |
| anti-IL23-SIRPaecd-SIGLEC10ecd | heavy chain = SEQ ID NO: 345 |
| (risankizumab) | light chain = SEQ ID NO: 339 |
| anti-IL23-SIRPaecd-TIM3ecd | heavy chain = SEQ ID NO: 345 |
| (risankizumab) | light chain = SEQ ID NO: 341 |
| anti-IL23-SIRPaecd | heavy chain = SEQ ID NO: 345 |
| (risankizumab) | light chain = SEQ ID NO: 75 |
| anti-IL23-TGFbRecd-BTLAecd | heavy chain = SEQ ID NO: 346 |
| (risankizumab) | light chain = SEQ ID NO: 343 |
| anti-IL23-TGFbRecd-PD1ecd | heavy chain = SEQ ID NO: 346 |
| (risankizumab) | light chain = SEQ ID NO: 338 |
| anti-IL23-TGFbRecd-SIGLEC10ecd | heavy chain = SEQ ID NO: 346 |
| (risankizumab) | light chain = SEQ ID NO: 339 |
| anti-IL23-TGFbRecd-SIRPaecd | heavy chain = SEQ ID NO: 346 |
| (risankizumab) | light chain = SEQ ID NO: 340 |
| anti-IL23-TGFbRecd-TIM3ecd | heavy chain = SEQ ID NO: 346 |
| (risankizumab) | light chain = SEQ ID NO: 341 |
| anti-IL23-TGFbRecd | heavy chain = SEQ ID NO: 346 |
| (risankizumab) | light chain = SEQ ID NO: 75 |
| anti-IL23-TIM3ecd-BTLAecd | heavy chain = SEQ ID NO: 347 |
| (risankizumab) | light chain = SEQ ID NO: 343 |
| anti-IL23-TIM3ecd-PD1ecd | heavy chain = SEQ ID NO: 347 |
| (risankizumab) | light chain = SEQ ID NO: 338 |
| anti-IL23-TIM3ecd-SIGLEC10ecd | heavy chain = SEQ ID NO: 347 |
| (risankizumab) | light chain = SEQ ID NO: 339 |
| anti-IL23-TIM3ecd-SIRPaecd | heavy chain = SEQ ID NO: 347 |
| (risankizumab) | light chain = SEQ ID NO: 340 |
| anti-IL23-TIM3ecd | heavy chain = SEQ ID NO: 347 |
| (risankizumab) | light chain = SEQ ID NO: 75 |
| anti-IL23-VEGFRecd-BTLAecd | heavy chain = SEQ ID NO: 348 |
| (risankizumab) | light chain = SEQ ID NO: 343 |
| anti-IL23-VEGFRecd-PD1ecd | heavy chain = SEQ ID NO: 348 |
| (risankizumab) | light chain = SEQ ID NO: 338 |
| anti-IL23-VEGFRecd-SIGLEC10ecd | heavy chain = SEQ ID NO: 348 |
| (risankizumab) | light chain = SEQ ID NO: 339 |
| anti-IL23-VEGFRecd-SIRPaecd | heavy chain = SEQ ID NO: 348 |
| (risankizumab) | light chain = SEQ ID NO: 340 |
| anti-IL23-VEGFRecd-TIM3ecd | heavy chain = SEQ ID NO: 348 |
| (risankizumab) | light chain = SEQ ID NO: 341 |
| anti-IL23-VEGFRecd | heavy chain = SEQ ID NO: 348 |
| (risankizumab) | light chain = SEQ ID NO: 75 |
| anti-IL1b-BTLAecd-PD1ecd | heavy chain = SEQ ID NO: 349 |
| (canakinumab) | light chain = SEQ ID NO: 350 |
| anti-IL1b-BTLAecd-SIGLEC10ecd | heavy chain = SEQ ID NO: 349 |
| (canakinumab) | light chain = SEQ ID NO: 351 |
| anti-IL1b-BTLAecd-SIRPaecd | heavy chain = SEQ ID NO: 349 |
| (canakinumab) | light chain = SEQ ID NO: 352 |
| anti-IL1b-BTLAecd-TIM3ecd | heavy chain = SEQ ID NO: 349 |
| (canakinumab) | light chain = SEQ ID NO: 353 |
| anti-IL1b-BTLAecd | heavy chain = SEQ ID NO: 349 |
| (canakinumab) | light chain = SEQ ID NO: 69 |
| anti-IL1b-PD1ecd-BTLAecd | heavy chain = SEQ ID NO: 354 |
| (canakinumab) | light chain = SEQ ID NO: 355 |
| anti-IL1b-PD1ecd-SIGLEC10ecd | heavy chain = SEQ ID NO: 354 |
| (canakinumab) | light chain = SEQ ID NO: 351 |
| anti-IL1b-PD1ecd-SIRPaecd | heavy chain = SEQ ID NO: 354 |
| (canakinumab) | light chain = SEQ ID NO: 352 |
| anti-IL1b-PD1ecd-TIM3ecd | heavy chain = SEQ ID NO: 354 |
| (canakinumab) | light chain = SEQ ID NO: 353 |
| anti-IL1b-PD1ecd | heavy chain = SEQ ID NO: 354 |
| (canakinumab) | light chain = SEQ ID NO: 69 |
| anti-IL1b-SIGLEC10ecd-BTLAecd | heavy chain = SEQ ID NO: 356 |
| (canakinumab) | light chain = SEQ ID NO: 355 |
| anti-IL1b-SIGLEC10ecd-PD1ecd | heavy chain = SEQ ID NO: 356 |
| (canakinumab) | light chain = SEQ ID NO: 350 |
| anti-IL1b-SIGLEC10ecd-SIRPaecd | heavy chain = SEQ ID NO: 356 |
| (canakinumab) | light chain = SEQ ID NO: 352 |
| anti-IL1b-SIGLEC10ecd-TIM3ecd | heavy chain = SEQ ID NO: 356 |
| (canakinumab) | light chain = SEQ ID NO: 353 |
| anti-IL1b-SIGLEC10ecd | heavy chain = SEQ ID NO: 356 |
| (canakinumab) | light chain = SEQ ID NO: 69 |
| anti-IL1b-SIRPaecd-BTLAecd | heavy chain = SEQ ID NO: 357 |
| (canakinumab) | light chain = SEQ ID NO: 355 |
| anti-IL1b-SIRPaecd-PD1ecd | heavy chain = SEQ ID NO: 357 |
| (canakinumab) | light chain = SEQ ID NO: 350 |
| anti-IL1b-SIRPaecd-SIGLEC10ecd | heavy chain = SEQ ID NO: 357 |
| (canakinumab) | light chain = SEQ ID NO: 351 |
| anti-IL1b-SIRPaecd-TIM3ecd | heavy chain = SEQ ID NO: 357 |
| (canakinumab) | light chain = SEQ ID NO: 353 |
| anti-IL1b-SIRPaecd | heavy chain = SEQ ID NO: 357 |
| (canakinumab) | light chain = SEQ ID NO: 69 |
| anti-IL1b-TGFbRecd-BTLAecd | heavy chain = SEQ ID NO: 358 |
| (canakinumab) | light chain = SEQ ID NO: 355 |
| anti-IL1b-TGFbRecd-PD1ecd | heavy chain = SEQ ID NO: 358 |
| (canakinumab) | light chain = SEQ ID NO: 350 |
| anti-IL1b-TGFbRecd-SIGLEC10ecd | heavy chain = SEQ ID NO: 358 |
| (canakinumab) | light chain = SEQ ID NO: 351 |
| anti-IL1b-TGFbRecd-SIRPaecd | heavy chain = SEQ ID NO: 358 |
| (canakinumab) | light chain = SEQ ID NO: 352 |
| anti-IL1b-TGFbRecd-TIM3ecd | heavy chain = SEQ ID NO: 358 |
| (canakinumab) | light chain = SEQ ID NO: 353 |
| anti-IL1b-TGFbRecd | heavy chain = SEQ ID NO: 358 |
| (canakinumab) | light chain = SEQ ID NO: 69 |
| anti-IL1b-TIM3ecd-BTLAecd | heavy chain = SEQ ID NO: 359 |
| (canakinumab) | light chain = SEQ ID NO: 355 |
| anti-IL1b-TIM3ecd-PD1ecd | heavy chain = SEQ ID NO: 359 |
| (canakinumab) | light chain = SEQ ID NO: 350 |
| anti-IL1b-TIM3ecd-SIGLEC10ecd | heavy chain = SEQ ID NO: 359 |
| (canakinumab) | light chain = SEQ ID NO: 351 |
| anti-IL1b-TIM3ecd-SIRPaecd | heavy chain = SEQ ID NO: 359 |
| (canakinumab) | light chain = SEQ ID NO: 352 |
| anti-IL1b-TIM3ecd | heavy chain = SEQ ID NO: 359 |
| (canakinumab) | light chain = SEQ ID NO: 69 |
| anti-IL1b-VEGFRecd-BTLAecd | heavy chain = SEQ ID NO: 360 |
| (canakinumab) | light chain = SEQ ID NO: 355 |
| anti-IL1b-VEGFRecd-PD1ecd | heavy chain = SEQ ID NO: 360 |
| (canakinumab) | light chain = SEQ ID NO: 350 |
| anti-IL1b-VEGFRecd-SIGLEC10ecd | heavy chain = SEQ ID NO: 360 |
| (canakinumab) | light chain = SEQ ID NO: 351 |
| anti-IL1b-VEGFRecd-SIRPaecd | heavy chain = SEQ ID NO: 360 |
| (canakinumab) | light chain = SEQ ID NO: 352 |
| anti-IL1b-VEGFRecd-TIM3ecd | heavy chain = SEQ ID NO: 360 |
| (canakinumab) | light chain = SEQ ID NO: 353 |
| anti-IL1b-VEGFRecd | heavy chain = SEQ ID NO: 360 |
| (canakinumab) | light chain = SEQ ID NO: 69 |
| anti-VEGF-BTLAecd-PD1ecd | heavy chain = SEQ ID NO: 361 |
| (bevacizumab) | light chain = SEQ ID NO: 362 |
| anti-VEGF-BTLAecd-SIGLEC10ecd | heavy chain = SEQ ID NO: 361 |
| (bevacizumab) | light chain = SEQ ID NO: 363 |
| anti-VEGF-BTLAecd-SIRPaecd | heavy chain = SEQ ID NO: 361 |
| (bevacizumab) | light chain = SEQ ID NO: 364 |
| anti-VEGF-BTLAecd-TIM3ecd | heavy chain = SEQ ID NO: 361 |
| (bevacizumab) | light chain = SEQ ID NO: 365 |
| anti-VEGF-BTLAecd | heavy chain = SEQ ID NO: 361 |
| (bevacizumab) | light chain = SEQ ID NO: 32 |
| anti-VEGF-PD1ecd-BTLAecd | heavy chain = SEQ ID NO: 366 |
| (bevacizumab) | light chain = SEQ ID NO: 367 |
| anti-VEGF-PD1ecd-SIGLEC10ecd | heavy chain = SEQ ID NO: 366 |
| (bevacizumab) | light chain = SEQ ID NO: 363 |
| anti-VEGF-PD1ecd-SIRPaecd | heavy chain = SEQ ID NO: 366 |
| (bevacizumab) | light chain = SEQ ID NO: 364 |
| anti-VEGF-PD1ecd-TIM3ecd | heavy chain = SEQ ID NO: 366 |
| (bevacizumab) | light chain = SEQ ID NO: 365 |
| anti-VEGF-PD1ecd | heavy chain = SEQ ID NO: 366 |
| (bevacizumab) | light chain = SEQ ID NO: 32 |
| anti-VEGF-SIGLEC10ecd-BTLAecd | heavy chain = SEQ ID NO: 368 |
| (bevacizumab) | light chain = SEQ ID NO: 367 |
| anti-VEGF-SIGLEC10ecd-PD1ecd | heavy chain = SEQ ID NO: 368 |
| (bevacizumab) | light chain = SEQ ID NO: 362 |
| anti-VEGF-SIGLEC10ecd-SIRPaecd | heavy chain = SEQ ID NO: 368 |
| (bevacizumab) | light chain = SEQ ID NO: 364 |
| anti-VEGF-SIGLEC10ecd-TIM3ecd | heavy chain = SEQ ID NO: 368 |
| (bevacizumab) | light chain = SEQ ID NO: 365 |
| anti-VEGF-SIGLEC10ecd | heavy chain = SEQ ID NO: 368 |
| (bevacizumab) | light chain = SEQ ID NO: 32 |
| anti-VEGF-SIRPaecd-BTLAecd | heavy chain = SEQ ID NO: 369 |
| (bevacizumab) | light chain = SEQ ID NO: 367 |
| anti-VEGF-SIRPaecd-PD1ecd | heavy chain = SEQ ID NO: 369 |
| (bevacizumab) | light chain = SEQ ID NO: 362 |
| anti-VEGF-SIRPaecd-SIGLEC10ecd | heavy chain = SEQ ID NO: 369 |
| (bevacizumab) | light chain = SEQ ID NO: 363 |
| anti-VEGF-SIRPaecd-TIM3ecd | heavy chain = SEQ ID NO: 369 |
| (bevacizumab) | light chain = SEQ ID NO: 365 |
| anti-VEGF-SIRPaecd | heavy chain = SEQ ID NO: 369 |
| (bevacizumab) | light chain = SEQ ID NO: 32 |
| anti-VEGF-TGFbRecd-BTLAecd | heavy chain = SEQ ID NO: 370 |
| (bevacizumab) | light chain = SEQ ID NO: 367 |
| anti-VEGF-TGFbRecd-PD1ecd | heavy chain = SEQ ID NO: 370 |
| (bevacizumab) | light chain = SEQ ID NO: 362 |
| anti-VEGF-TGFbRecd-SIGLEC10ecd | heavy chain = SEQ ID NO: 370 |
| (bevacizumab) | light chain = SEQ ID NO: 363 |
| anti-VEGF-TGFbRecd-SIRPaecd | heavy chain = SEQ ID NO: 370 |
| (bevacizumab) | light chain = SEQ ID NO: 364 |
| anti-VEGF-TGFbRecd-TIM3ecd | heavy chain = SEQ ID NO: 370 |
| (bevacizumab) | light chain = SEQ ID NO: 365 |
| anti-VEGF-TGFbRecd | heavy chain = SEQ ID NO: 370 |
| (bevacizumab) | light chain = SEQ ID NO: 32 |
| anti-VEGF-TIM3ecd-BTLAecd | heavy chain = SEQ ID NO: 371 |
| (bevacizumab) | light chain = SEQ ID NO: 367 |
| anti-VEGF-TIM3ecd-PD1ecd | heavy chain = SEQ ID NO: 371 |
| (bevacizumab) | light chain = SEQ ID NO: 362 |
| anti-VEGF-TIM3ecd-SIGLEC10ecd | heavy chain = SEQ ID NO: 371 |
| (bevacizumab) | light chain = SEQ ID NO: 363 |
| anti-VEGF-TIM3ecd-SIRPaecd | heavy chain = SEQ ID NO: 371 |
| (bevacizumab) | light chain = SEQ ID NO: 364 |
| anti-VEGF-TIM3ecd | heavy chain = SEQ ID NO: 371 |
| (bevacizumab) | light chain = SEQ ID NO: 32 |
| anti-VEGFR-BTLAecd-PD1ecd | heavy chain = SEQ ID NO: 372 |
| (ramucirumab) | light chain = SEQ ID NO: 373 |
| anti-VEGFR-BTLAecd-SIGLEC10ecd | heavy chain = SEQ ID NO: 372 |
| (ramucirumab) | light chain = SEQ ID NO: 374 |
| anti-VEGFR-BTLAecd-SIRPaecd | heavy chain = SEQ ID NO: 372 |
| (ramucirumab) | light chain = SEQ ID NO: 375 |
| anti-VEGFR-BTLAecd-TIM3ecd | heavy chain = SEQ ID NO: 372 |
| (ramucirumab) | light chain = SEQ ID NO: 376 |
| anti-VEGFR-BTLAecd | heavy chain = SEQ ID NO: 372 |
| (ramucirumab) | light chain = SEQ ID NO: 148 |
| anti-VEGFR-PD1ecd-BTLAecd | heavy chain = SEQ ID NO: 377 |
| (ramucirumab) | light chain = SEQ ID NO: 378 |
| anti-VEGFR-PD1ecd-SIGLEC10ecd | heavy chain = SEQ ID NO: 377 |
| (ramucirumab) | light chain = SEQ ID NO: 374 |
| anti-VEGFR-PD1ecd-SIRPaecd | heavy chain = SEQ ID NO: 377 |
| (ramucirumab) | light chain = SEQ ID NO: 375 |
| anti-VEGFR-PD1ecd-TIM3ecd | heavy chain = SEQ ID NO: 377 |
| (ramucirumab) | light chain = SEQ ID NO: 376 |
| anti-VEGFR-PD1ecd | heavy chain = SEQ ID NO: 377 |
| (ramucirumab) | light chain = SEQ ID NO: 148 |
| anti-VEGFR-SIGLEC10ecd-BTLAecd | heavy chain = SEQ ID NO: 379 |
| (ramucirumab) | light chain = SEQ ID NO: 378 |
| anti-VEGFR-SIGLEC10ecd-PD1ecd | heavy chain = SEQ ID NO: 379 |
| (ramucirumab) | light chain = SEQ ID NO: 373 |
| anti-VEGFR-SIGLEC10ecd-SIRPaecd | heavy chain = SEQ ID NO: 379 |
| (ramucirumab) | light chain = SEQ ID NO: 375 |
| anti-VEGFR-SIGLEC10ecd-TIM3ecd | heavy chain = SEQ ID NO: 379 |
| (ramucirumab) | light chain = SEQ ID NO: 376 |
| anti-VEGFR-SIGLEC10ecd | heavy chain = SEQ ID NO: 379 |
| (ramucirumab) | light chain = SEQ ID NO: 148 |
| anti-VEGFR-SIRPaecd-BTLAecd | heavy chain = SEQ ID NO: 380 |
| (ramucirumab) | light chain = SEQ ID NO: 378 |
| anti-VEGFR-SIRPaecd-PD1ecd | heavy chain = SEQ ID NO: 380 |
| (ramucirumab) | light chain = SEQ ID NO: 373 |
| anti-VEGFR-SIRPaecd-SIGLEC10ecd | heavy chain = SEQ ID NO: 380 |
| (ramucirumab) | light chain = SEQ ID NO: 374 |
| anti-VEGFR-SIRPaecd-TIM3ecd | heavy chain = SEQ ID NO: 380 |
| (ramucirumab) | light chain = SEQ ID NO: 376 |
| anti-VEGFR-SIRPaecd | heavy chain = SEQ ID NO: 380 |
| (ramucirumab) | light chain = SEQ ID NO: 148 |
| anti-VEGFR-TGFbRecd-BTLAecd | heavy chain = SEQ ID NO: 381 |
| (ramucirumab) | light chain = SEQ ID NO: 378 |
| anti-VEGFR-TGFbRecd-PD1ecd | heavy chain = SEQ ID NO: 381 |
| (ramucirumab) | light chain = SEQ ID NO: 373 |
| anti-VEGFR-TGFbRecd-SIGLEC10ecd | heavy chain = SEQ ID NO: 381 |
| (ramucirumab) | light chain = SEQ ID NO: 374 |
| anti-VEGFR-TGFbRecd-SIRPaecd | heavy chain = SEQ ID NO: 381 |
| (ramucirumab) | light chain = SEQ ID NO: 375 |
| anti-VEGFR-TGFbRecd-TIM3ecd | heavy chain = SEQ ID NO: 381 |
| (ramucirumab) | light chain = SEQ ID NO: 376 |
| anti-VEGFR-TGFbRecd | heavy chain = SEQ ID NO: 381 |
| (ramucirumab) | light chain = SEQ ID NO: 148 |
| anti-VEGFR-TIM3ecd-BTLAecd | heavy chain = SEQ ID NO: 382 |
| (ramucirumab) | light chain = SEQ ID NO: 378 |
| anti-VEGFR-TIM3ecd-PD1ecd | heavy chain = SEQ ID NO: 382 |
| (ramucirumab) | light chain = SEQ ID NO: 373 |
| anti-VEGFR-TIM3ecd-SIGLEC10ecd | heavy chain = SEQ ID NO: 382 |
| (ramucirumab) | light chain = SEQ ID NO: 374 |
| anti-VEGFR-TIM3ecd-SIRPaecd | heavy chain = SEQ ID NO: 382 |
| (ramucirumab) | light chain = SEQ ID NO: 375 |
| anti-VEGFR-TIM3ecd | heavy chain = SEQ ID NO: 382 |
| (ramucirumab) | light chain = SEQ ID NO: 148 |
| anti-CD47-BTLAecd-PD1ecd | heavy chain = SEQ ID NO: 383 |
| (5F9) | light chain = SEQ ID NO: 384 |
| anti-CD47-BTLAecd-SIGLEC10ecd | heavy chain = SEQ ID NO: 383 |
| (5F9) | light chain = SEQ ID NO: 385 |
| anti-CD47-BTLAecd-TIM3ecd | heavy chain = SEQ ID NO: 383 |
| (5F9) | light chain = SEQ ID NO: 386 |
| anti-CD47-BTLAecd | heavy chain = SEQ ID NO: 383 |
| (5F9) | light chain = SEQ ID NO: 22 |
| anti-CD47-PD1ecd-BTLAecd | heavy chain = SEQ ID NO: 387 |
| (5F9) | light chain = SEQ ID NO: 388 |
| anti-CD47-PD1ecd-SIGLEC10ecd | heavy chain = SEQ ID NO: 387 |
| (5F9) | light chain = SEQ ID NO: 385 |
| anti-CD47-PD1ecd-TIM3ecd | heavy chain = SEQ ID NO: 387 |
| (5F9) | light chain = SEQ ID NO: 386 |
| anti-CD47-PD1ecd | heavy chain = SEQ ID NO: 387 |
| (5F9) | light chain = SEQ ID NO: 22 |
| anti-CD47-SIGLEC10ecd-BTLAecd | heavy chain = SEQ ID NO: 389 |
| (5F9) | light chain = SEQ ID NO: 388 |
| anti-CD47-SIGLEC10ecd-PD1ecd | heavy chain = SEQ ID NO: 389 |
| (5F9) | light chain = SEQ ID NO: 384 |
| anti-CD47-SIGLEC10ecd-TIM3ecd | heavy chain = SEQ ID NO: 389 |
| (5F9) | light chain = SEQ ID NO: 386 |
| anti-CD47-SIGLEC10ecd | heavy chain = SEQ ID NO: 389 |
| (5F9) | light chain = SEQ ID NO: 22 |
| anti-CD47-TGFbRecd-BTLAecd | heavy chain = SEQ ID NO: 390 |
| (5F9) | light chain = SEQ ID NO: 388 |
| anti-CD47-TGFbRecd-PD1ecd | heavy chain = SEQ ID NO: 390 |
| (5F9) | light chain = SEQ ID NO: 384 |
| anti-CD47-TGFbRecd-SIGLEC10ecd | heavy chain = SEQ ID NO: 390 |
| (5F9) | light chain = SEQ ID NO: 385 |
| anti-CD47-TGFbRecd-TIM3ecd | heavy chain = SEQ ID NO: 390 |
| (5F9) | light chain = SEQ ID NO: 386 |
| anti-CD47-TGFbRecd | heavy chain = SEQ ID NO: 390 |
| (5F9) | light chain = SEQ ID NO: 22 |
| anti-CD47-TIM3ecd-BTLAecd | heavy chain = SEQ ID NO: 391 |
| (5F9) | light chain = SEQ ID NO: 388 |
| anti-CD47-TIM3ecd-PD1ecd | heavy chain = SEQ ID NO: 391 |
| (5F9) | light chain = SEQ ID NO: 384 |
| anti-CD47-TIM3ecd-SIGLEC10ecd | heavy chain = SEQ ID NO: 391 |
| (5F9) | light chain = SEQ ID NO: 385 |
| anti-CD47-TIM3ecd | heavy chain = SEQ ID NO: 391 |
| (5F9) | light chain = SEQ ID NO: 22 |
| anti-CD47-VEGFRecd-BTLAecd | heavy chain = SEQ ID NO: 392 |
| (5F9) | light chain = SEQ ID NO: 388 |
| anti-CD47-VEGFRecd-PD1ecd | heavy chain = SEQ ID NO: 392 |
| (5F9) | light chain = SEQ ID NO: 384 |
| anti-CD47-VEGFRecd-SIGLEC10ecd | heavy chain = SEQ ID NO: 392 |
| (5F9) | light chain = SEQ ID NO: 385 |
| anti-CD47-VEGFRecd-TIM3ecd | heavy chain = SEQ ID NO: 392 |
| (5F9) | light chain = SEQ ID NO: 386 |
| anti-CD47-VEGFRecd | heavy chain = SEQ ID NO: 392 |
| (5F9) | light chain = SEQ ID NO: 22 |
| anti-TGFb-BTLAecd-PD1ecd | heavy chain = SEQ ID NO: 393 |
| (fresolimumab) | light chain = SEQ ID NO: 394 |
| anti-TGFb-BTLAecd-SIGLEC10ecd | heavy chain = SEQ ID NO: 393 |
| (fresolimumab) | light chain = SEQ ID NO: 395 |
| anti-TGFb-BTLAecd-SIRPaecd | heavy chain = SEQ ID NO: 393 |
| (fresolimumab) | light chain = SEQ ID NO: 396 |
| anti-TGFb-BTLAecd-TIM3ecd | heavy chain = SEQ ID NO: 393 |
| (fresolimumab) | light chain = SEQ ID NO: 397 |
| anti-TGFb-BTLAecd | heavy chain = SEQ ID NO: 393 |
| (fresolimumab) | light chain = SEQ ID NO: 133 |
| anti-TGFb-PD1ecd-BTLAecd | heavy chain = SEQ ID NO: 398 |
| (fresolimumab) | light chain = SEQ ID NO: 399 |
| anti-TGFb-PD1ecd-SIGLEC10ecd | heavy chain = SEQ ID NO: 398 |
| (fresolimumab) | light chain = SEQ ID NO: 395 |
| anti-TGFb-PD1ecd-SIRPaecd | heavy chain = SEQ ID NO: 398 |
| (fresolimumab) | light chain = SEQ ID NO: 396 |
| anti-TGFb-PD1ecd-TIM3ecd | heavy chain = SEQ ID NO: 398 |
| (fresolimumab) | light chain = SEQ ID NO: 397 |
| anti-TGFb-PD1ecd | heavy chain = SEQ ID NO: 398 |
| (fresolimumab) | light chain = SEQ ID NO: 133 |
| anti-TGFb-SIGLEC10ecd-BTLAecd | heavy chain = SEQ ID NO: 400 |
| (fresolimumab) | light chain = SEQ ID NO: 399 |
| anti-TGFb-SIGLEC10ecd-PD1ecd | heavy chain = SEQ ID NO: 400 |
| (fresolimumab) | light chain = SEQ ID NO: 394 |
| anti-TGFb-SIGLEC10ecd-SIRPaecd | heavy chain = SEQ ID NO: 400 |
| (fresolimumab) | light chain = SEQ ID NO: 396 |
| anti-TGFb-SIGLEC10ecd-TIM3ecd | heavy chain = SEQ ID NO: 400 |
| (fresolimumab) | light chain = SEQ ID NO: 397 |
| anti-TGFb-SIGLEC10ecd | heavy chain = SEQ ID NO: 400 |
| (fresolimumab) | light chain = SEQ ID NO: 133 |
| anti-TGFb-SIRPaecd-BTLAecd | heavy chain = SEQ ID NO: 401 |
| (fresolimumab) | light chain = SEQ ID NO: 399 |
| anti-TGFb-SIRPaecd-PD1ecd | heavy chain = SEQ ID NO: 401 |
| (fresolimumab) | light chain = SEQ ID NO: 394 |
| anti-TGFb-SIRPaecd-SIGLEC10ecd | heavy chain = SEQ ID NO: 401 |
| (fresolimumab) | light chain = SEQ ID NO: 395 |
| anti-TGFb-SIRPaecd-TIM3ecd | heavy chain = SEQ ID NO: 401 |
| (fresolimumab) | light chain = SEQ ID NO: 397 |
| anti-TGFb-SIRPaecd | heavy chain = SEQ ID NO: 401 |
| (fresolimumab) | light chain = SEQ ID NO: 133 |
| anti-TGFb-TIM3ecd-BTLAecd | heavy chain = SEQ ID NO: 402 |
| (fresolimumab) | light chain = SEQ ID NO: 399 |
| anti-TGFb-TIM3ecd-PD1ecd | heavy chain = SEQ ID NO: 402 |
| (fresolimumab) | light chain = SEQ ID NO: 394 |
| anti-TGFb-TIM3ecd-SIGLEC10ecd | heavy chain = SEQ ID NO: 402 |
| (fresolimumab) | light chain = SEQ ID NO: 395 |
| anti-TGFb-TIM3ecd-SIRPaecd | heavy chain = SEQ ID NO: 402 |
| (fresolimumab) | light chain = SEQ ID NO: 396 |
| anti-TGFb-TIM3ecd | heavy chain = SEQ ID NO: 402 |
| (fresolimumab) | light chain = SEQ ID NO: 133 |
| anti-TGFb-VEGFRecd-BTLAecd | heavy chain = SEQ ID NO: 403 |
| (fresolimumab) | light chain = SEQ ID NO: 399 |
| anti-TGFb-VEGFRecd-PD1ecd | heavy chain = SEQ ID NO: 403 |
| (fresolimumab) | light chain = SEQ ID NO: 394 |
| anti-TGFb-VEGFRecd-SIGLEC10ecd | heavy chain = SEQ ID NO: 403 |
| (fresolimumab) | light chain = SEQ ID NO: 395 |
| anti-TGFb-VEGFRecd-SIRPaecd | heavy chain = SEQ ID NO: 403 |
| (fresolimumab) | light chain = SEQ ID NO: 396 |
| anti-TGFb-VEGFRecd-TIM3ecd | heavy chain = SEQ ID NO: 403 |
| (fresolimumab) | light chain = SEQ ID NO: 397 |
| anti-TGFb-VEGFRecd | heavy chain = SEQ ID NO: 403 |
| (fresolimumab) | light chain = SEQ ID NO: 133 |
| anti-LAP-BTLAecd | heavy chain = SEQ ID NO: 404 |
| (7H4) | light chain = SEQ ID NO: 81 |
| anti-LAP-PD1ecd | heavy chain = SEQ ID NO: 405 |
| (7H4) | light chain = SEQ ID NO: 81 |
| anti-LAP-SIGLEC10ecd | heavy chain = SEQ ID NO: 406 |
| (7H4) | light chain = SEQ ID NO: 81 |
| anti-LAP-SIRPaecd | heavy chain = SEQ ID NO: 407 |
| (7H4) | light chain = SEQ ID NO: 81 |
| anti-LAP-TIM3ecd | heavy chain = SEQ ID NO: 408 |
| (7H4) | light chain = SEQ ID NO: 81 |
| anti-LAP-VEGFRecd | heavy chain = SEQ ID NO: 409 |
| (7H4) | light chain = SEQ ID NO: 81 |
| anti-GARP-BTLAecd | heavy chain = SEQ ID NO: 410 |
| (ARGX-115) | light chain = SEQ ID NO: 49 |
| anti-GARP-PD1ecd | heavy chain = SEQ ID NO: 411 |
| (ARGX-115) | light chain = SEQ ID NO: 49 |
| anti-GARP-SIGLEC10ecd | heavy chain = SEQ ID NO: 412 |
| (ARGX-115) | light chain = SEQ ID NO: 49 |
| anti-GARP-SIRPaecd | heavy chain = SEQ ID NO: 413 |
| (ARGX-115) | light chain = SEQ ID NO: 49 |
| anti-GARP-TIM3ecd | heavy chain = SEQ ID NO: 414 |
| (ARGX-115) | light chain = SEQ ID NO: 49 |
| anti-GARP-VEGFRecd | heavy chain = SEQ ID NO: 415 |
| (ARGX-115) | light chain = SEQ ID NO: 49 |
| anti-CD73-BTLAecd-PD1ecd | heavy chain = SEQ ID NO: 416 |
| (GS1423) | light chain = SEQ ID NO: 417 |
| anti-CD73-BTLAecd-SIGLEC10ecd | heavy chain = SEQ ID NO: 416 |
| (GS1423) | light chain = SEQ ID NO: 418 |
| anti-CD73-BTLAecd-SIRPaecd | heavy chain = SEQ ID NO: 416 |
| (GS1423) | light chain = SEQ ID NO: 419 |
| anti-CD73-BTLAecd-TIM3ecd | heavy chain = SEQ ID NO: 416 |
| (GS1423) | light chain = SEQ ID NO: 420 |
| anti-CD73-BTLAecd | heavy chain = SEQ ID NO: 416 |
| (GS1423) | light chain = SEQ ID NO: 24 |
| anti-CD73-PD1ecd-BTLAecd | heavy chain = SEQ ID NO: 421 |
| (GS1423) | light chain = SEQ ID NO: 422 |
| anti-CD73-PD1ecd-SIGLEC10ecd | heavy chain = SEQ ID NO: 421 |
| (GS1423) | light chain = SEQ ID NO: 418 |
| anti-CD73-PD1ecd-SIRPaecd | heavy chain = SEQ ID NO: 421 |
| (GS1423) | light chain = SEQ ID NO: 419 |
| anti-CD73-PD1ecd-TIM3ecd | heavy chain = SEQ ID NO: 421 |
| (GS1423) | light chain = SEQ ID NO: 420 |
| anti-CD73-PD1ecd | heavy chain = SEQ ID NO: 421 |
| (GS1423) | light chain = SEQ ID NO: 24 |
| anti-CD73-SIGLEC10ecd-BTLAecd | heavy chain = SEQ ID NO: 423 |
| (GS1423) | light chain = SEQ ID NO: 422 |
| anti-CD73-SIGLEC10ecd-PD1ecd | heavy chain = SEQ ID NO: 423 |
| (GS1423) | light chain = SEQ ID NO: 417 |
| anti-CD73-SIGLEC10ecd-SIRPaecd | heavy chain = SEQ ID NO: 423 |
| (GS1423) | light chain = SEQ ID NO: 419 |
| anti-CD73-SIGLEC10ecd-TIM3ecd | heavy chain = SEQ ID NO: 423 |
| (GS1423) | light chain = SEQ ID NO: 420 |
| anti-CD73-SIGLEC10ecd | heavy chain = SEQ ID NO: 423 |
| (GS1423) | light chain = SEQ ID NO: 24 |
| anti-CD73-SIRPaecd-BTLAecd | heavy chain = SEQ ID NO: 424 |
| (GS1423) | light chain = SEQ ID NO: 422 |
| anti-CD73-SIRPaecd-PD1ecd | heavy chain = SEQ ID NO: 424 |
| (GS1423) | light chain = SEQ ID NO: 417 |
| anti-CD73-SIRPaecd-SIGLEC10ecd | heavy chain = SEQ ID NO: 424 |
| (GS1423) | light chain = SEQ ID NO: 418 |
| anti-CD73-SIRPaecd-TIM3ecd | heavy chain = SEQ ID NO: 424 |
| (GS1423) | light chain = SEQ ID NO: 420 |
| anti-CD73-SIRPaecd | heavy chain = SEQ ID NO: 424 |
| (GS1423) | light chain = SEQ ID NO: 24 |
| anti-CD73-TGFbRecd-BTLAecd | heavy chain = SEQ ID NO: 425 |
| (GS1423) | light chain = SEQ ID NO: 422 |
| anti-CD73-TGFbRecd-PD1ecd | heavy chain = SEQ ID NO: 425 |
| (GS1423) | light chain = SEQ ID NO: 417 |
| anti-CD73-TGFbRecd-SIGLEC10ecd | heavy chain = SEQ ID NO: 425 |
| (GS1423) | light chain = SEQ ID NO: 418 |
| anti-CD73-TGFbRecd-SIRPaecd | heavy chain = SEQ ID NO: 425 |
| (GS1423) | light chain = SEQ ID NO: 419 |
| anti-CD73-TGFbRecd-TIM3ecd | heavy chain = SEQ ID NO: 425 |
| (GS1423) | light chain = SEQ ID NO: 420 |
| anti-CD73-TGFbRecd | heavy chain = SEQ ID NO: 425 |
| (GS1423) | light chain = SEQ ID NO: 24 |
| anti-CD73-TIM3ecd-BTLAecd | heavy chain = SEQ ID NO: 426 |
| (GS1423) | light chain = SEQ ID NO: 422 |
| anti-CD73-TIM3ecd-PD1ecd | heavy chain = SEQ ID NO: 426 |
| (GS1423) | light chain = SEQ ID NO: 417 |
| anti-CD73-TIM3ecd-SIGLEC10ecd | heavy chain = SEQ ID NO: 426 |
| (GS1423) | light chain = SEQ ID NO: 418 |
| anti-CD73-TIM3ecd-SIRPaecd | heavy chain = SEQ ID NO: 426 |
| (GS1423) | light chain = SEQ ID NO: 419 |
| anti-CD73-TIM3ecd | heavy chain = SEQ ID NO: 426 |
| (GS1423) | light chain = SEQ ID NO: 24 |
| anti-CD73-VEGFRecd-BTLAecd | heavy chain = SEQ ID NO: 427 |
| (GS1423) | light chain = SEQ ID NO: 422 |
| anti-CD73-VEGFRecd-PD1ecd | heavy chain = SEQ ID NO: 427 |
| (GS1423) | light chain = SEQ ID NO: 417 |
| anti-CD73-VEGFRecd-SIGLEC10ecd | heavy chain = SEQ ID NO: 427 |
| (GS1423) | light chain = SEQ ID NO: 418 |
| anti-CD73-VEGFRecd-SIRPaecd | heavy chain = SEQ ID NO: 427 |
| (GS1423) | light chain = SEQ ID NO: 419 |
| anti-CD73-VEGFRecd-TIM3ecd | heavy chain = SEQ ID NO: 427 |
| (GS1423) | light chain = SEQ ID NO: 420 |
| anti-CD73-VEGFRecd | heavy chain = SEQ ID NO: 427 |
| (GS1423) | light chain = SEQ ID NO: 24 |
| anti-CD39-BTLAecd | heavy chain = SEQ ID NO: 428 |
| (IPH5201) | light chain = SEQ ID NO: 18 |
| anti-CD39-PD1ecd | heavy chain = SEQ ID NO: 429 |
| (IPH5201) | light chain = SEQ ID NO: 18 |
| anti-CD39-SIGLEC10ecd | heavy chain = SEQ ID NO: 430 |
| (IPH5201) | light chain = SEQ ID NO: 18 |
| anti-CD39-SIRPaecd | heavy chain = SEQ ID NO: 431 |
| (IPH5201) | light chain = SEQ ID NO: 18 |
| anti-CD39-TGFbRecd | heavy chain = SEQ ID NO: 432 |
| (IPH5201) | light chain = SEQ ID NO: 18 |
| anti-CD39-TIM3ecd | heavy chain = SEQ ID NO: 433 |
| (IPH5201) | light chain = SEQ ID NO: 18 |
| anti-CD39-VEGFRecd | heavy chain = SEQ ID NO: 434 |
| (IPH5201) | light chain = SEQ ID NO: 18 |
| anti-CTLA4-BTLAecd-PD1ecd | heavy chain = SEQ ID NO: 435 |
| (ipilimumab) | light chain = SEQ ID NO: 436 |
| anti-CTLA4-BTLAecd-SIGLEC10ecd | heavy chain = SEQ ID NO: 435 |
| (ipilimumab) | light chain = SEQ ID NO: 437 |
| anti-CTLA4-BTLAecd-SIRPaecd | heavy chain = SEQ ID NO: 435 |
| (ipilimumab) | light chain = SEQ ID NO: 438 |
| anti-CTLA4-BTLAecd-TIM3ecd | heavy chain = SEQ ID NO: 435 |
| (ipilimumab) | light chain = SEQ ID NO: 439 |
| anti-CTLA4-BTLAecd | heavy chain = SEQ ID NO: 435 |
| (ipilimumab) | light chain = SEQ ID NO: 28 |
| anti-CTLA4-PD1ecd-BTLAecd | heavy chain = SEQ ID NO: 440 |
| (ipilimumab) | light chain = SEQ ID NO: 441 |
| anti-CTLA4-PD1ecd-SIGLEC10ecd | heavy chain = SEQ ID NO: 440 |
| (ipilimumab) | light chain = SEQ ID NO: 437 |
| anti-CTLA4-PD1ecd-SIRPaecd | heavy chain = SEQ ID NO: 440 |
| (ipilimumab) | light chain = SEQ ID NO: 438 |
| anti-CTLA4-PD1ecd-TIM3ecd | heavy chain = SEQ ID NO: 440 |
| (ipilimumab) | light chain = SEQ ID NO: 439 |
| anti-CTLA4-PD1ecd | heavy chain = SEQ ID NO: 440 |
| (ipilimumab) | light chain = SEQ ID NO: 28 |
| anti-CTLA4-SIGLEC10ecd-BTLAecd | heavy chain = SEQ ID NO: 442 |
| (ipilimumab) | light chain = SEQ ID NO: 441 |
| anti-CTLA4-SIGLEC10ecd-PD1ecd | heavy chain = SEQ ID NO: 442 |
| (ipilimumab) | light chain = SEQ ID NO: 436 |
| anti-CTLA4-SIGLEC10ecd-SIRPaecd | heavy chain = SEQ ID NO: 442 |
| (ipilimumab) | light chain = SEQ ID NO: 438 |
| anti-CTLA4-SIGLEC10ecd-TIM3ecd | heavy chain = SEQ ID NO: 442 |
| (ipilimumab) | light chain = SEQ ID NO: 439 |
| anti-CTLA4-SIGLEC10ecd | heavy chain = SEQ ID NO: 442 |
| (ipilimumab) | light chain = SEQ ID NO: 28 |
| anti-CTLA4-SIRPaecd-BTLAecd | heavy chain = SEQ ID NO: 443 |
| (ipilimumab) | light chain = SEQ ID NO: 441 |
| anti-CTLA4-SIRPaecd-PD1ecd | heavy chain = SEQ ID NO: 443 |
| (ipilimumab) | light chain = SEQ ID NO: 436 |
| anti-CTLA4-SIRPaecd-SIGLEC10ecd | heavy chain = SEQ ID NO: 443 |
| (ipilimumab) | light chain = SEQ ID NO: 437 |
| anti-CTLA4-SIRPaecd-TIM3ecd | heavy chain = SEQ ID NO: 443 |
| (ipilimumab) | light chain = SEQ ID NO: 439 |
| anti-CTLA4-SIRPaecd | heavy chain = SEQ ID NO: 443 |
| (ipilimumab) | light chain = SEQ ID NO: 28 |
| anti-CTLA4-TGFbRecd-BTLAecd | heavy chain = SEQ ID NO: 444 |
| (ipilimumab) | light chain = SEQ ID NO: 441 |
| anti-CTLA4-TGFbRecd-PD1ecd | heavy chain = SEQ ID NO: 444 |
| (ipilimumab) | light chain = SEQ ID NO: 436 |
| anti-CTLA4-TGFbRecd-SIGLEC10ecd | heavy chain = SEQ ID NO: 444 |
| (ipilimumab) | light chain = SEQ ID NO: 437 |
| anti-CTLA4-TGFbRecd-SIRPaecd | heavy chain = SEQ ID NO: 444 |
| (ipilimumab) | light chain = SEQ ID NO: 438 |
| anti-CTLA4-TGFbRecd-TIM3ecd | heavy chain = SEQ ID NO: 444 |
| (ipilimumab) | light chain = SEQ ID NO: 439 |
| anti-CTLA4-TGFbRecd | heavy chain = SEQ ID NO: 444 |
| (ipilimumab) | light chain = SEQ ID NO: 28 |
| anti-CTLA4-TIM3ecd-BTLAecd | heavy chain = SEQ ID NO: 445 |
| (ipilimumab) | light chain = SEQ ID NO: 441 |
| anti-CTLA4-TIM3ecd-PD1ecd | heavy chain = SEQ ID NO: 445 |
| (ipilimumab) | light chain = SEQ ID NO: 436 |
| anti-CTLA4-TIM3ecd-SIGLEC10ecd | heavy chain = SEQ ID NO: 445 |
| (ipilimumab) | light chain = SEQ ID NO: 437 |
| anti-CTLA4-TIM3ecd-SIRPaecd | heavy chain = SEQ ID NO: 445 |
| (ipilimumab) | light chain = SEQ ID NO: 438 |
| anti-CTLA4-TIM3ecd | heavy chain = SEQ ID NO: 445 |
| (ipilimumab) | light chain = SEQ ID NO: 28 |
| anti-CTLA4-VEGFRecd-BTLAecd | heavy chain = SEQ ID NO: 446 |
| (ipilimumab) | light chain = SEQ ID NO: 441 |
| anti-CTLA4-VEGFRecd-PD1ecd | heavy chain = SEQ ID NO: 446 |
| (ipilimumab) | light chain = SEQ ID NO: 436 |
| anti-CTLA4-VEGFRecd-SIGLEC10ecd | heavy chain = SEQ ID NO: 446 |
| (ipilimumab) | light chain = SEQ ID NO: 437 |
| anti-CTLA4-VEGFRecd-SIRPaecd | heavy chain = SEQ ID NO: 446 |
| (ipilimumab) | light chain = SEQ ID NO: 438 |
| anti-CTLA4-VEGFRecd-TIM3ecd | heavy chain = SEQ ID NO: 446 |
| (ipilimumab) | light chain = SEQ ID NO: 439 |
| anti-CTLA4-VEGFRecd | heavy chain = SEQ ID NO: 446 |
| (ipilimumab) | light chain = SEQ ID NO: 28 |
| anti-PD1-BTLAecd-PD1ecd | heavy chain = SEQ ID NO: 447 |
| (pembrolizumab) | light chain = SEQ ID NO: 448 |
| anti-PD1-BTLAecd-SIGLEC10ecd | heavy chain = SEQ ID NO: 447 |
| (pembrolizumab) | light chain = SEQ ID NO: 449 |
| anti-PD1-BTLAecd-SIRPaecd | heavy chain = SEQ ID NO: 447 |
| (pembrolizumab) | light chain = SEQ ID NO: 450 |
| anti-PD1-BTLAecd-TIM3ecd | heavy chain = SEQ ID NO: 447 |
| (pembrolizumab) | light chain = SEQ ID NO: 451 |
| anti-PD1-BTLAecd | heavy chain = SEQ ID NO: 447 |
| (pembrolizumab) | light chain = SEQ ID NO: 101 |
| anti-PD1-PD1ecd-BTLAecd | heavy chain = SEQ ID NO: 452 |
| (pembrolizumab) | light chain = SEQ ID NO: 453 |
| anti-PD1-PD1ecd-SIGLEC10ecd | heavy chain = SEQ ID NO: 452 |
| (pembrolizumab) | light chain = SEQ ID NO: 449 |
| anti-PD1-PD1ecd-SIRPaecd | heavy chain = SEQ ID NO: 452 |
| (pembrolizumab) | light chain = SEQ ID NO: 450 |
| anti-PD1-PD1ecd-TIM3ecd | heavy chain = SEQ ID NO: 452 |
| (pembrolizumab) | light chain = SEQ ID NO: 451 |
| anti-PD1-PD1ecd | heavy chain = SEQ ID NO: 452 |
| (pembrolizumab) | light chain = SEQ ID NO: 101 |
| anti-PD1-SIGLEC10ecd-BTLAecd | heavy chain = SEQ ID NO: 454 |
| (pembrolizumab) | light chain = SEQ ID NO: 453 |
| anti-PD1-SIGLEC10ecd-PD1ecd | heavy chain = SEQ ID NO: 454 |
| (pembrolizumab) | light chain = SEQ ID NO: 448 |
| anti-PD1-SIGLEC10ecd-SIRPaecd | heavy chain = SEQ ID NO: 454 |
| (pembrolizumab) | light chain = SEQ ID NO: 450 |
| anti-PD1-SIGLEC10ecd-TIM3ecd | heavy chain = SEQ ID NO: 454 |
| (pembrolizumab) | light chain = SEQ ID NO: 451 |
| anti-PD1-SIGLEC10ecd | heavy chain = SEQ ID NO: 454 |
| (pembrolizumab) | light chain = SEQ ID NO: 101 |
| anti-PD1-SIRPaecd-BTLAecd | heavy chain = SEQ ID NO: 455 |
| (pembrolizumab) | light chain = SEQ ID NO: 453 |
| anti-PD1-SIRPaecd-PD1ecd | heavy chain = SEQ ID NO: 455 |
| (pembrolizumab) | light chain = SEQ ID NO: 448 |
| anti-PD1-SIRPaecd-SIGLEC10ecd | heavy chain = SEQ ID NO: 455 |
| (pembrolizumab) | light chain = SEQ ID NO: 449 |
| anti-PD1-SIRPaecd-TIM3ecd | heavy chain = SEQ ID NO: 455 |
| (pembrolizumab) | light chain = SEQ ID NO: 451 |
| anti-PD1-SIRPaecd | heavy chain = SEQ ID NO: 455 |
| (pembrolizumab) | light chain = SEQ ID NO: 101 |
| anti-PD1-TGFbRecd-BTLAecd | heavy chain = SEQ ID NO: 456 |
| (pembrolizumab) | light chain = SEQ ID NO: 453 |
| anti-PD1-TGFbRecd-PD1ecd | heavy chain = SEQ ID NO: 456 |
| (pembrolizumab) | light chain = SEQ ID NO: 448 |
| anti-PD1-TGFbRecd-SIGLEC10ecd | heavy chain = SEQ ID NO: 456 |
| (pembrolizumab) | light chain = SEQ ID NO: 449 |
| anti-PD1-TGFbRecd-SIRPaecd | heavy chain = SEQ ID NO: 456 |
| (pembrolizumab) | light chain = SEQ ID NO: 450 |
| anti-PD1-TGFbRecd-TIM3ecd | heavy chain = SEQ ID NO: 456 |
| (pembrolizumab) | light chain = SEQ ID NO: 451 |
| anti-PD1-TGFbRecd | heavy chain = SEQ ID NO: 456 |
| (pembrolizumab) | light chain = SEQ ID NO: 101 |
| anti-PD1-TIM3ecd-BTLAecd | heavy chain = SEQ ID NO: 457 |
| (pembrolizumab) | light chain = SEQ ID NO: 453 |
| anti-PD1-TIM3ecd-PD1ecd | heavy chain = SEQ ID NO: 457 |
| (pembrolizumab) | light chain = SEQ ID NO: 448 |
| anti-PD1-TIM3ecd-SIGLEC10ecd | heavy chain = SEQ ID NO: 457 |
| (pembrolizumab) | light chain = SEQ ID NO: 449 |
| anti-PD1-TIM3ecd-SIRPaecd | heavy chain = SEQ ID NO: 457 |
| (pembrolizumab) | light chain = SEQ ID NO: 450 |
| anti-PD1-TIM3ecd | heavy chain = SEQ ID NO: 457 |
| (pembrolizumab) | light chain = SEQ ID NO: 101 |
| anti-PD1-VEGFRecd-BTLAecd | heavy chain = SEQ ID NO: 458 |
| (pembrolizumab) | light chain = SEQ ID NO: 453 |
| anti-PD1-VEGFRecd-PD1ecd | heavy chain = SEQ ID NO: 458 |
| (pembrolizumab) | light chain = SEQ ID NO: 448 |
| anti-PD1-VEGFRecd-SIGLEC10ecd | heavy chain = SEQ ID NO: 458 |
| (pembrolizumab) | light chain = SEQ ID NO: 449 |
| anti-PD1-VEGFRecd-SIRPaecd | heavy chain = SEQ ID NO: 458 |
| (pembrolizumab) | light chain = SEQ ID NO: 450 |
| anti-PD1-VEGFRecd-TIM3ecd | heavy chain = SEQ ID NO: 458 |
| (pembrolizumab) | light chain = SEQ ID NO: 451 |
| anti-PD1-VEGFRecd | heavy chain = SEQ ID NO: 458 |
| (pembrolizumab) | light chain = SEQ ID NO: 101 |
| anti-PDL1-BTLAecd-SIGLEC10ecd | heavy chain = SEQ ID NO: 459 |
| (atezolizumab) | light chain = SEQ ID NO: 460 |
| anti-PDL1-BTLAecd-SIRPaecd | heavy chain = SEQ ID NO: 459 |
| (atezolizumab) | light chain = SEQ ID NO: 461 |
| anti-PDL1-BTLAecd-TIM3ecd | heavy chain = SEQ ID NO: 459 |
| (atezolizumab) | light chain = SEQ ID NO: 462 |
| anti-PDL1-BTLAecd | heavy chain = SEQ ID NO: 459 |
| (atezolizumab) | light chain = SEQ ID NO: 109 |
| anti-PDL1-SIGLEC10ecd-BTLAecd | heavy chain = SEQ ID NO: 463 |
| (atezolizumab) | light chain = SEQ ID NO: 464 |
| anti-PDL1-SIGLEC10ecd-SIRPaecd | heavy chain = SEQ ID NO: 463 |
| (atezolizumab) | light chain = SEQ ID NO: 461 |
| anti-PDL1-SIGLEC10ecd-TIM3ecd | heavy chain = SEQ ID NO: 463 |
| (atezolizumab) | light chain = SEQ ID NO: 462 |
| anti-PDL1-SIGLEC10ecd | heavy chain = SEQ ID NO: 463 |
| (atezolizumab) | light chain = SEQ ID NO: 109 |
| anti-PDL1-SIRPaecd-BTLAecd | heavy chain = SEQ ID NO: 465 |
| (atezolizumab) | light chain = SEQ ID NO: 464 |
| anti-PDL1-SIRPaecd-SIGLEC10ecd | heavy chain = SEQ ID NO: 465 |
| (atezolizumab) | light chain = SEQ ID NO: 460 |
| anti-PDL1-SIRPaecd-TIM3ecd | heavy chain = SEQ ID NO: 465 |
| (atezolizumab) | light chain = SEQ ID NO: 462 |
| anti-PDL1-SIRPaecd | heavy chain = SEQ ID NO: 465 |
| (atezolizumab) | light chain = SEQ ID NO: 109 |
| anti-PDL1-TGFbRecd-BTLAecd | heavy chain = SEQ ID NO: 466 |
| (atezolizumab) | light chain = SEQ ID NO: 464 |
| anti-PDL1-TGFbRecd-SIGLEC10ecd | heavy chain = SEQ ID NO: 466 |
| (atezolizumab) | light chain = SEQ ID NO: 460 |
| anti-PDL1-TGFbRecd-SIRPaecd | heavy chain = SEQ ID NO: 466 |
| (atezolizumab) | light chain = SEQ ID NO: 461 |
| anti-PDL1-TGFbRecd-TIM3ecd | heavy chain = SEQ ID NO: 466 |
| (atezolizumab) | light chain = SEQ ID NO: 462 |
| anti-PDL1-TGFbRecd | heavy chain = SEQ ID NO: 466 |
| (atezolizumab) | light chain = SEQ ID NO: 109 |
| anti-PDL1-TIM3ecd-BTLAecd | heavy chain = SEQ ID NO: 467 |
| (atezolizumab) | light chain = SEQ ID NO: 464 |
| anti-PDL1-TIM3ecd-SIGLEC10ecd | heavy chain = SEQ ID NO: 467 |
| (atezolizumab) | light chain = SEQ ID NO: 460 |
| anti-PDL1-TIM3ecd-SIRPaecd | heavy chain = SEQ ID NO: 467 |
| (atezolizumab) | light chain = SEQ ID NO: 461 |
| anti-PDL1-TIM3ecd | heavy chain = SEQ ID NO: 467 |
| (atezolizumab) | light chain = SEQ ID NO: 109 |
| anti-PDL1-VEGFRecd-BTLAecd | heavy chain = SEQ ID NO: 468 |
| (atezolizumab) | light chain = SEQ ID NO: 464 |
| anti-PDL1-VEGFRecd-SIGLEC10ecd | heavy chain = SEQ ID NO: 468 |
| (atezolizumab) | light chain = SEQ ID NO: 460 |
| anti-PDL1-VEGFRecd-SIRPaecd | heavy chain = SEQ ID NO: 468 |
| (atezolizumab) | light chain = SEQ ID NO: 461 |
| anti-PDL1-VEGFRecd-TIM3ecd | heavy chain = SEQ ID NO: 468 |
| (atezolizumab) | light chain = SEQ ID NO: 462 |
| anti-PDL1-VEGFRecd | heavy chain = SEQ ID NO: 468 |
| (atezolizumab) | light chain = SEQ ID NO: 109 |
| anti-TIGIT-BTLAecd-PD1ecd | heavy chain = SEQ ID NO: 469 |
| (tiragolumab) | light chain = SEQ ID NO: 470 |
| anti-TIGIT-BTLAecd-SIGLEC10ecd | heavy chain = SEQ ID NO: 469 |
| (tiragolumab) | light chain = SEQ ID NO: 471 |
| anti-TIGIT-BTLAecd-SIRPaecd | heavy chain = SEQ ID NO: 469 |
| (tiragolumab) | light chain = SEQ ID NO: 472 |
| anti-TIGIT-BTLAecd-TIM3ecd | heavy chain = SEQ ID NO: 469 |
| (tiragolumab) | light chain = SEQ ID NO: 473 |
| anti-TIGIT-BTLAecd | heavy chain = SEQ ID NO: 469 |
| (tiragolumab) | light chain = SEQ ID NO: 139 |
| anti-TIGIT-PD1ecd-BTLAecd | heavy chain = SEQ ID NO: 474 |
| (tiragolumab) | light chain = SEQ ID NO: 475 |
| anti-TIGIT-PD1ecd-SIGLEC10ecd | heavy chain = SEQ ID NO: 474 |
| (tiragolumab) | light chain = SEQ ID NO: 471 |
| anti-TIGIT-PD1ecd-SIRPaecd | heavy chain = SEQ ID NO: 474 |
| (tiragolumab) | light chain = SEQ ID NO: 472 |
| anti-TIGIT-PD1ecd-TIM3ecd | heavy chain = SEQ ID NO: 474 |
| (tiragolumab) | light chain = SEQ ID NO: 473 |
| anti-TIGIT-PD1ecd | heavy chain = SEQ ID NO: 474 |
| (tiragolumab) | light chain = SEQ ID NO: 139 |
| anti-TIGIT-SIGLEC10ecd-BTLAecd | heavy chain = SEQ ID NO: 476 |
| (tiragolumab) | light chain = SEQ ID NO: 475 |
| anti-TIGIT-SIGLEC10ecd-PD1ecd | heavy chain = SEQ ID NO: 476 |
| (tiragolumab) | light chain = SEQ ID NO: 470 |
| anti-TIGIT-SIGLEC10ecd-SIRPaecd | heavy chain = SEQ ID NO: 476 |
| (tiragolumab) | light chain = SEQ ID NO: 472 |
| anti-TIGIT-SIGLEC10ecd-TIM3ecd | heavy chain = SEQ ID NO: 476 |
| (tiragolumab) | light chain = SEQ ID NO: 473 |
| anti-TIGIT-SIGLEC10ecd | heavy chain = SEQ ID NO: 476 |
| (tiragolumab) | light chain = SEQ ID NO: 139 |
| anti-TIGIT-SIRPaecd-BTLAecd | heavy chain = SEQ ID NO: 477 |
| (tiragolumab) | light chain = SEQ ID NO: 475 |
| anti-TIGIT-SIRPaecd-PD1ecd | heavy chain = SEQ ID NO: 477 |
| (tiragolumab) | light chain = SEQ ID NO: 470 |
| anti-TIGIT-SIRPaecd-SIGLEC10ecd | heavy chain = SEQ ID NO: 477 |
| (tiragolumab) | light chain = SEQ ID NO: 471 |
| anti-TIGIT-SIRPaecd-TIM3ecd | heavy chain = SEQ ID NO: 477 |
| (tiragolumab) | light chain = SEQ ID NO: 473 |
| anti-TIGIT-SIRPaecd | heavy chain = SEQ ID NO: 477 |
| (tiragolumab) | light chain = SEQ ID NO: 139 |
| anti-TIGIT-TGFbRecd-BTLAecd | heavy chain = SEQ ID NO: 478 |
| (tiragolumab) | light chain = SEQ ID NO: 475 |
| anti-TIGIT-TGFbRecd-PD1ecd | heavy chain = SEQ ID NO: 478 |
| (tiragolumab) | light chain = SEQ ID NO: 470 |
| anti-TIGIT-TGFbRecd-SIGLEC10ecd | heavy chain = SEQ ID NO: 478 |
| (tiragolumab) | light chain = SEQ ID NO: 471 |
| anti-TIGIT-TGFbRecd-SIRPaecd | heavy chain = SEQ ID NO: 478 |
| (tiragolumab) | light chain = SEQ ID NO: 472 |
| anti-TIGIT-TGFbRecd-TIM3ecd | heavy chain = SEQ ID NO: 478 |
| (tiragolumab) | light chain = SEQ ID NO: 473 |
| anti-TIGIT-TGFbRecd | heavy chain = SEQ ID NO: 478 |
| (tiragolumab) | light chain = SEQ ID NO: 139 |
| anti-TIGIT-TIM3ecd-BTLAecd | heavy chain = SEQ ID NO: 479 |
| (tiragolumab) | light chain = SEQ ID NO: 475 |
| anti-TIGIT-TIM3ecd-PD1ecd | heavy chain = SEQ ID NO: 479 |
| (tiragolumab) | light chain = SEQ ID NO: 470 |
| anti-TIGIT-TIM3ecd-SIGLEC10ecd | heavy chain = SEQ ID NO: 479 |
| (tiragolumab) | light chain = SEQ ID NO: 471 |
| anti-TIGIT-TIM3ecd-SIRPaecd | heavy chain = SEQ ID NO: 479 |
| (tiragolumab) | light chain = SEQ ID NO: 472 |
| anti-TIGIT-TIM3ecd | heavy chain = SEQ ID NO: 479 |
| (tiragolumab) | light chain = SEQ ID NO: 139 |
| anti-TIGIT-VEGFRecd-BTLAecd | heavy chain = SEQ ID NO: 480 |
| (tiragolumab) | light chain = SEQ ID NO: 475 |
| anti-TIGIT-VEGFRecd-PD1ecd | heavy chain = SEQ ID NO: 480 |
| (tiragolumab) | light chain = SEQ ID NO: 470 |
| anti-TIGIT-VEGFRecd-SIGLEC10ecd | heavy chain = SEQ ID NO: 480 |
| (tiragolumab) | light chain = SEQ ID NO: 471 |
| anti-TIGIT-VEGFRecd-SIRPaecd | heavy chain = SEQ ID NO: 480 |
| (tiragolumab) | light chain = SEQ ID NO: 472 |
| anti-TIGIT-VEGFRecd-TIM3ecd | heavy chain = SEQ ID NO: 480 |
| (tiragolumab) | light chain = SEQ ID NO: 473 |
| anti-TIGIT-VEGFRecd | heavy chain = SEQ ID NO: 480 |
| (tiragolumab) | light chain = SEQ ID NO: 139 |
| anti-TIM3-BTLAecd-PD1ecd | heavy chain = SEQ ID NO: 481 |
| (M6903) | light chain = SEQ ID NO: 482 |
| anti-TIM3-BTLAecd-SIGLEC10ecd | heavy chain = SEQ ID NO: 481 |
| (M6903) | light chain = SEQ ID NO: 483 |
| anti-TIM3-BTLAecd-SIRPaecd | heavy chain = SEQ ID NO: 481 |
| (M6903) | light chain = SEQ ID NO: 484 |
| anti-TIM3-BTLAecd-TIM3ecd | heavy chain = SEQ ID NO: 481 |
| (M6903) | light chain = SEQ ID NO: 485 |
| anti-TIM3-BTLAecd | heavy chain = SEQ ID NO: 481 |
| (M6903) | light chain = SEQ ID NO: 141 |
| anti-TIM3-PD1ecd-BTLAecd | heavy chain = SEQ ID NO: 486 |
| (M6903) | light chain = SEQ ID NO: 487 |
| anti-TIM3-PD1ecd-SIGLEC10ecd | heavy chain = SEQ ID NO: 486 |
| (M6903) | light chain = SEQ ID NO: 483 |
| anti-TIM3-PD1ecd-SIRPaecd | heavy chain = SEQ ID NO: 486 |
| (M6903) | light chain = SEQ ID NO: 484 |
| anti-TIM3-PD1ecd-TIM3ecd | heavy chain = SEQ ID NO: 486 |
| (M6903) | light chain = SEQ ID NO: 485 |
| anti-TIM3-PD1ecd | heavy chain = SEQ ID NO: 486 |
| (M6903) | light chain = SEQ ID NO: 141 |
| anti-TIM3-SIGLEC10ecd-BTLAecd | heavy chain = SEQ ID NO: 488 |
| (M6903) | light chain = SEQ ID NO: 487 |
| anti-TIM3-SIGLEC10ecd-PD1ecd | heavy chain = SEQ ID NO: 488 |
| (M6903) | light chain = SEQ ID NO: 482 |
| anti-TIM3-SIGLEC10ecd-SIRPaecd | heavy chain = SEQ ID NO: 488 |
| (M6903) | light chain = SEQ ID NO: 484 |
| anti-TIM3-SIGLEC10ecd-TIM3ecd | heavy chain = SEQ ID NO: 488 |
| (M6903) | light chain = SEQ ID NO: 485 |
| anti-TIM3-SIGLEC10ecd | heavy chain = SEQ ID NO: 488 |
| (M6903) | light chain = SEQ ID NO: 141 |
| anti-TIM3-SIRPaecd-BTLAecd | heavy chain = SEQ ID NO: 489 |
| (M6903) | light chain = SEQ ID NO: 487 |
| anti-TIM3-SIRPaecd-PD1ecd | heavy chain = SEQ ID NO: 489 |
| (M6903) | light chain = SEQ ID NO: 482 |
| anti-TIM3-SIRPaecd-SIGLEC10ecd | heavy chain = SEQ ID NO: 489 |
| (M6903) | light chain = SEQ ID NO: 483 |
| anti-TIM3-SIRPaecd-TIM3ecd | heavy chain = SEQ ID NO: 489 |
| (M6903) | light chain = SEQ ID NO: 485 |
| anti-TIM3-SIRPaecd | heavy chain = SEQ ID NO: 489 |
| (M6903) | light chain = SEQ ID NO: 141 |
| anti-TIM3-TGFbRecd-BTLAecd | heavy chain = SEQ ID NO: 490 |
| (M6903) | light chain = SEQ ID NO: 487 |
| anti-TIM3-TGFbRecd-PD1ecd | heavy chain = SEQ ID NO: 490 |
| (M6903) | light chain = SEQ ID NO: 482 |
| anti-TIM3-TGFbRecd-SIGLEC10ecd | heavy chain = SEQ ID NO: 490 |
| (M6903) | light chain = SEQ ID NO: 483 |
| anti-TIM3-TGFbRecd-SIRPaecd | heavy chain = SEQ ID NO: 490 |
| (M6903) | light chain = SEQ ID NO: 484 |
| anti-TIM3-TGFbRecd-TIM3ecd | heavy chain = SEQ ID NO: 490 |
| (M6903) | light chain = SEQ ID NO: 485 |
| anti-TIM3-TGFbRecd | heavy chain = SEQ ID NO: 490 |
| (M6903) | light chain = SEQ ID NO: 141 |
| anti-TIM3-TIM3ecd-BTLAecd | heavy chain = SEQ ID NO: 491 |
| (M6903) | light chain = SEQ ID NO: 487 |
| anti-TIM3-TIM3ecd-PD1ecd | heavy chain = SEQ ID NO: 491 |
| (M6903) | light chain = SEQ ID NO: 482 |
| anti-TIM3-TIM3ecd-SIGLEC10ecd | heavy chain = SEQ ID NO: 491 |
| (M6903) | light chain = SEQ ID NO: 483 |
| anti-TIM3-TIM3ecd-SIRPaecd | heavy chain = SEQ ID NO: 491 |
| (M6903) | light chain = SEQ ID NO: 484 |
| anti-TIM3-TIM3ecd | heavy chain = SEQ ID NO: 491 |
| (M6903) | light chain = SEQ ID NO: 141 |
| anti-TIM3-VEGFRecd-BTLAecd | heavy chain = SEQ ID NO: 492 |
| (M6903) | light chain = SEQ ID NO: 487 |
| anti-TIM3-VEGFRecd-PD1ecd | heavy chain = SEQ ID NO: 492 |
| (M6903) | light chain = SEQ ID NO: 482 |
| anti-TIM3-VEGFRecd-SIGLEC10ecd | heavy chain = SEQ ID NO: 492 |
| (M6903) | light chain = SEQ ID NO: 483 |
| anti-TIM3-VEGFRecd-SIRPaecd | heavy chain = SEQ ID NO: 492 |
| (M6903) | light chain = SEQ ID NO: 484 |
| anti-TIM3-VEGFRecd-TIM3ecd | heavy chain = SEQ ID NO: 492 |
| (M6903) | light chain = SEQ ID NO: 485 |
| anti-TIM3-VEGFRecd | heavy chain = SEQ ID NO: 492 |
| (M6903) | light chain = SEQ ID NO: 141 |
| anti-41BB-BTLAecd-PD1ecd | heavy chain = SEQ ID NO: 493 |
| (urelumab) | light chain = SEQ ID NO: 494 |
| anti-41BB-BTLAecd-SIGLEC10ecd | heavy chain = SEQ ID NO: 493 |
| (urelumab) | light chain = SEQ ID NO: 495 |
| anti-41BB-BTLAecd-SIRPaecd | heavy chain = SEQ ID NO: 493 |
| (urelumab) | light chain = SEQ ID NO: 496 |
| anti-41BB-BTLAecd-TIM3ecd | heavy chain = SEQ ID NO: 493 |
| (urelumab) | light chain = SEQ ID NO: 497 |
| anti-41BB-BTLAecd | heavy chain = SEQ ID NO: 493 |
| (urelumab) | light chain = SEQ ID NO: 2 |
| anti-41BB-PD1ecd-BTLAecd | heavy chain = SEQ ID NO: 498 |
| (urelumab) | light chain = SEQ ID NO: 499 |
| anti-41BB-PD1ecd-SIGLEC10ecd | heavy chain = SEQ ID NO: 498 |
| (urelumab) | light chain = SEQ ID NO: 495 |
| anti-41BB-PD1ecd-SIRPaecd | heavy chain = SEQ ID NO: 498 |
| (urelumab) | light chain = SEQ ID NO: 496 |
| anti-41BB-PD1ecd-TIM3ecd | heavy chain = SEQ ID NO: 498 |
| (urelumab) | light chain = SEQ ID NO: 497 |
| anti-41BB-PD1ecd | heavy chain = SEQ ID NO: 498 |
| (urelumab) | light chain = SEQ ID NO: 2 |
| anti-41BB-SIGLEC10ecd-BTLAecd | heavy chain = SEQ ID NO: 500 |
| (urelumab) | light chain = SEQ ID NO: 499 |
| anti-41BB-SIGLEC10ecd-PD1ecd | heavy chain = SEQ ID NO: 500 |
| (urelumab) | light chain = SEQ ID NO: 494 |
| anti-41BB-SIGLEC10ecd-SIRPaecd | heavy chain = SEQ ID NO: 500 |
| (urelumab) | light chain = SEQ ID NO: 496 |
| anti-41BB-SIGLEC10ecd-TIM3ecd | heavy chain = SEQ ID NO: 500 |
| (urelumab) | light chain = SEQ ID NO: 497 |
| anti-41BB-SIGLEC10ecd | heavy chain = SEQ ID NO: 500 |
| (urelumab) | light chain = SEQ ID NO: 2 |
| anti-41BB-SIRPaecd-BTLAecd | heavy chain = SEQ ID NO: 501 |
| (urelumab) | light chain = SEQ ID NO: 499 |
| anti-41BB-SIRPaecd-PD1ecd | heavy chain = SEQ ID NO: 501 |
| (urelumab) | light chain = SEQ ID NO: 494 |
| anti-41BB-SIRPaecd-SIGLEC10ecd | heavy chain = SEQ ID NO: 501 |
| (urelumab) | light chain = SEQ ID NO: 495 |
| anti-41BB-SIRPaecd-TIM3ecd | heavy chain = SEQ ID NO: 501 |
| (urelumab) | light chain = SEQ ID NO: 497 |
| anti-41BB-SIRPaecd | heavy chain = SEQ ID NO: 501 |
| (urelumab) | light chain = SEQ ID NO: 2 |
| anti-41BB-TGFbRecd-BTLAecd | heavy chain = SEQ ID NO: 502 |
| (urelumab) | light chain = SEQ ID NO: 499 |
| anti-41BB-TGFbRecd-PD1ecd | heavy chain = SEQ ID NO: 502 |
| (urelumab) | light chain = SEQ ID NO: 494 |
| anti-41BB-TGFbRecd-SIGLEC10ecd | heavy chain = SEQ ID NO: 502 |
| (urelumab) | light chain = SEQ ID NO: 495 |
| anti-41BB-TGFbRecd-SIRPaecd | heavy chain = SEQ ID NO: 502 |
| (urelumab) | light chain = SEQ ID NO: 496 |
| anti-41BB-TGFbRecd-TIM3ecd | heavy chain = SEQ ID NO: 502 |
| (urelumab) | light chain = SEQ ID NO: 497 |
| anti-41BB-TGFbRecd | heavy chain = SEQ ID NO: 502 |
| (urelumab) | light chain = SEQ ID NO: 2 |
| anti-41BB-TIM3ecd-BTLAecd | heavy chain = SEQ ID NO: 503 |
| (urelumab) | light chain = SEQ ID NO: 499 |
| anti-41BB-TIM3ecd-PD1ecd | heavy chain = SEQ ID NO: 503 |
| (urelumab) | light chain = SEQ ID NO: 494 |
| anti-41BB-TIM3ecd-SIGLEC10ecd | heavy chain = SEQ ID NO: 503 |
| (urelumab) | light chain = SEQ ID NO: 495 |
| anti-41BB-TIM3ecd-SIRPaecd | heavy chain = SEQ ID NO: 503 |
| (urelumab) | light chain = SEQ ID NO: 496 |
| anti-41BB-TIM3ecd | heavy chain = SEQ ID NO: 503 |
| (urelumab) | light chain = SEQ ID NO: 2 |
| anti-41BB-VEGFRecd-BTLAecd | heavy chain = SEQ ID NO: 504 |
| (urelumab) | light chain = SEQ ID NO: 499 |
| anti-41BB-VEGFRecd-PD1ecd | heavy chain = SEQ ID NO: 504 |
| (urelumab) | light chain = SEQ ID NO: 494 |
| anti-41BB-VEGFRecd-SIGLEC10ecd | heavy chain = SEQ ID NO: 504 |
| (urelumab) | light chain = SEQ ID NO: 495 |
| anti-41BB-VEGFRecd-SIRPaecd | heavy chain = SEQ ID NO: 504 |
| (urelumab) | light chain = SEQ ID NO: 496 |
| anti-41BB-VEGFRecd-TIM3ecd | heavy chain = SEQ ID NO: 504 |
| (urelumab) | light chain = SEQ ID NO: 497 |
| anti-41BB-VEGFRecd | heavy chain = SEQ ID NO: 504 |
| (urelumab) | light chain = SEQ ID NO: 2 |
| anti-OX40-BTLAecd-PD1ecd | heavy chain = SEQ ID NO: 505 |
| (GSK3174998) | light chain = SEQ ID NO: 506 |
| anti-OX40-BTLAecd-SIGLEC10ecd | heavy chain = SEQ ID NO: 505 |
| (GSK3174998) | light chain = SEQ ID NO: 507 |
| anti-OX40-BTLAecd-SIRPaecd | heavy chain = SEQ ID NO: 505 |
| (GSK3174998) | light chain = SEQ ID NO: 508 |
| anti-OX40-BTLAecd-TIM3ecd | heavy chain = SEQ ID NO: 505 |
| (GSK3174998) | light chain = SEQ ID NO: 509 |
| anti-OX40-BTLAecd | heavy chain = SEQ ID NO: 505 |
| (GSK3174998) | light chain = SEQ ID NO: 97 |
| anti-OX40-PD1ecd-BTLAecd | heavy chain = SEQ ID NO: 510 |
| (GSK3174998) | light chain = SEQ ID NO: 511 |
| anti-OX40-PD1ecd-SIGLEC10ecd | heavy chain = SEQ ID NO: 510 |
| (GSK3174998) | light chain = SEQ ID NO: 507 |
| anti-OX40-PD1ecd-SIRPaecd | heavy chain = SEQ ID NO: 510 |
| (GSK3174998) | light chain = SEQ ID NO: 508 |
| anti-OX40-PD1ecd-TIM3ecd | heavy chain = SEQ ID NO: 510 |
| (GSK3174998) | light chain = SEQ ID NO: 509 |
| anti-OX40-PD1ecd | heavy chain = SEQ ID NO: 510 |
| (GSK3174998) | light chain = SEQ ID NO: 97 |
| anti-OX40-SIGLEC10ecd-BTLAecd | heavy chain = SEQ ID NO: 512 |
| (GSK3174998) | light chain = SEQ ID NO: 511 |
| anti-OX40-SIGLEC10ecd-PD1ecd | heavy chain = SEQ ID NO: 512 |
| (GSK3174998) | light chain = SEQ ID NO: 506 |
| anti-OX40-SIGLEC10ecd-SIRPaecd | heavy chain = SEQ ID NO: 512 |
| (GSK3174998) | light chain = SEQ ID NO: 508 |
| anti-OX40-SIGLEC10ecd-TIM3ecd | heavy chain = SEQ ID NO: 512 |
| (GSK3174998) | light chain = SEQ ID NO: 509 |
| anti-OX40-SIGLEC10ecd | heavy chain = SEQ ID NO: 512 |
| (GSK3174998) | light chain = SEQ ID NO: 97 |
| anti-OX40-SIRPaecd-BTLAecd | heavy chain = SEQ ID NO: 513 |
| (GSK3174998) | light chain = SEQ ID NO: 511 |
| anti-OX40-SIRPaecd-PD1ecd | heavy chain = SEQ ID NO: 513 |
| (GSK3174998) | light chain = SEQ ID NO: 506 |
| anti-OX40-SIRPaecd-SIGLEC10ecd | heavy chain = SEQ ID NO: 513 |
| (GSK3174998) | light chain = SEQ ID NO: 507 |
| anti-OX40-SIRPaecd-TIM3ecd | heavy chain = SEQ ID NO: 513 |
| (GSK3174998) | light chain = SEQ ID NO: 509 |
| anti-OX40-SIRPaecd | heavy chain = SEQ ID NO: 513 |
| (GSK3174998) | light chain = SEQ ID NO: 97 |
| anti-OX40-TGFbRecd-BTLAecd | heavy chain = SEQ ID NO: 514 |
| (GSK3174998) | light chain = SEQ ID NO: 511 |
| anti-OX40-TGFbRecd-PD1ecd | heavy chain = SEQ ID NO: 514 |
| (GSK3174998) | light chain = SEQ ID NO: 506 |
| anti-OX40-TGFbRecd-SIGLEC10ecd | heavy chain = SEQ ID NO: 514 |
| (GSK3174998) | light chain = SEQ ID NO: 507 |
| anti-OX40-TGFbRecd-SIRPaecd | heavy chain = SEQ ID NO: 514 |
| (GSK3174998) | light chain = SEQ ID NO: 508 |
| anti-OX40-TGFbRecd-TIM3ecd | heavy chain = SEQ ID NO: 514 |
| (GSK3174998) | light chain = SEQ ID NO: 509 |
| anti-OX40-TGFbRecd | heavy chain = SEQ ID NO: 514 |
| (GSK3174998) | light chain = SEQ ID NO: 97 |
| anti-OX40-TIM3ecd-BTLAecd | heavy chain = SEQ ID NO: 515 |
| (GSK3174998) | light chain = SEQ ID NO: 511 |
| anti-OX40-TIM3ecd-PD1ecd | heavy chain = SEQ ID NO: 515 |
| (GSK3174998) | light chain = SEQ ID NO: 506 |
| anti-OX40-TIM3ecd-SIGLEC10ecd | heavy chain = SEQ ID NO: 515 |
| (GSK3174998) | light chain = SEQ ID NO: 507 |
| anti-OX40-TIM3ecd-SIRPaecd | heavy chain = SEQ ID NO: 515 |
| (GSK3174998) | light chain = SEQ ID NO: 508 |
| anti-OX40-TIM3ecd | heavy chain = SEQ ID NO: 515 |
| (GSK3174998) | light chain = SEQ ID NO: 97 |
| anti-OX40-VEGFRecd-BTLAecd | heavy chain = SEQ ID NO: 516 |
| (GSK3174998) | light chain = SEQ ID NO: 511 |
| anti-OX40-VEGFRecd-PD1ecd | heavy chain = SEQ ID NO: 516 |
| (GSK3174998) | light chain = SEQ ID NO: 506 |
| anti-OX40-VEGFRecd-SIGLEC10ecd | heavy chain = SEQ ID NO: 516 |
| (GSK3174998) | light chain = SEQ ID NO: 507 |
| anti-OX40-VEGFRecd-SIRPaecd | heavy chain = SEQ ID NO: 516 |
| (GSK3174998) | light chain = SEQ ID NO: 508 |
| anti-OX40-VEGFRecd-TIM3ecd | heavy chain = SEQ ID NO: 516 |
| (GSK3174998) | light chain = SEQ ID NO: 509 |
| anti-OX40-VEGFRecd | heavy chain = SEQ ID NO: 516 |
| (GSK3174998) | light chain = SEQ ID NO: 97 |
| anti-ICOS-BTLAecd-PD1ecd | heavy chain = SEQ ID NO: 517 |
| (GSK3359609) | light chain = SEQ ID NO: 518 |
| anti-ICOS-BTLAecd-SIGLEC10ecd | heavy chain = SEQ ID NO: 517 |
| (GSK3359609) | light chain = SEQ ID NO: 519 |
| anti-ICOS-BTLAecd-SIRPaecd | heavy chain = SEQ ID NO: 517 |
| (GSK3359609) | light chain = SEQ ID NO: 520 |
| anti-ICOS-BTLAecd-TIM3ecd | heavy chain = SEQ ID NO: 517 |
| (GSK3359609) | light chain = SEQ ID NO: 521 |
| anti-ICOS-BTLAecd | heavy chain = SEQ ID NO: 517 |
| (GSK3359609) | light chain = SEQ ID NO: 59 |
| anti-ICOS-PD1ecd-BTLAecd | heavy chain = SEQ ID NO: 522 |
| (GSK3359609) | light chain = SEQ ID NO: 523 |
| anti-ICOS-PD1ecd-SIGLEC10ecd | heavy chain = SEQ ID NO: 522 |
| (GSK3359609) | light chain = SEQ ID NO: 519 |
| anti-ICOS-PD1ecd-SIRPaecd | heavy chain = SEQ ID NO: 522 |
| (GSK3359609) | light chain = SEQ ID NO: 520 |
| anti-ICOS-PD1ecd-TIM3ecd | heavy chain = SEQ ID NO: 522 |
| (GSK3359609) | light chain = SEQ ID NO: 521 |
| anti-ICOS-PD1ecd | heavy chain = SEQ ID NO: 522 |
| (GSK3359609) | light chain = SEQ ID NO: 59 |
| anti-ICOS-SIGLEC10ecd-BTLAecd | heavy chain = SEQ ID NO: 524 |
| (GSK3359609) | light chain = SEQ ID NO: 523 |
| anti-ICOS-SIGLEC10ecd-PD1ecd | heavy chain = SEQ ID NO: 524 |
| (GSK3359609) | light chain = SEQ ID NO: 518 |
| anti-ICOS-SIGLEC10ecd-SIRPaecd | heavy chain = SEQ ID NO: 524 |
| (GSK3359609) | light chain = SEQ ID NO: 520 |
| anti-ICOS-SIGLEC10ecd-TIM3ecd | heavy chain = SEQ ID NO: 524 |
| (GSK3359609) | light chain = SEQ ID NO: 521 |
| anti-ICOS-SIGLEC10ecd | heavy chain = SEQ ID NO: 524 |
| (GSK3359609) | light chain = SEQ ID NO: 59 |
| anti-ICOS-SIRPaecd-BTLAecd | heavy chain = SEQ ID NO: 525 |
| (GSK3359609) | light chain = SEQ ID NO: 523 |
| anti-ICOS-SIRPaecd-PD1ecd | heavy chain = SEQ ID NO: 525 |
| (GSK3359609) | light chain = SEQ ID NO: 518 |
| anti-ICOS-SIRPaecd-SIGLEC10ecd | heavy chain = SEQ ID NO: 525 |
| (GSK3359609) | light chain = SEQ ID NO: 519 |
| anti-ICOS-SIRPaecd-TIM3ecd | heavy chain = SEQ ID NO: 525 |
| (GSK3359609) | light chain = SEQ ID NO: 521 |
| anti-ICOS-SIRPaecd | heavy chain = SEQ ID NO: 525 |
| (GSK3359609) | light chain = SEQ ID NO: 59 |
| anti-ICOS-TGFbRecd-BTLAecd | heavy chain = SEQ ID NO: 526 |
| (GSK3359609) | light chain = SEQ ID NO: 523 |
| anti-ICOS-TGFbRecd-PD1ecd | heavy chain = SEQ ID NO: 526 |
| (GSK3359609) | light chain = SEQ ID NO: 518 |
| anti-ICOS-TGFbRecd-SIGLEC10ecd | heavy chain = SEQ ID NO: 526 |
| (GSK3359609) | light chain = SEQ ID NO: 519 |
| anti-ICOS-TGFbRecd-SIRPaecd | heavy chain = SEQ ID NO: 526 |
| (GSK3359609) | light chain = SEQ ID NO: 520 |
| anti-ICOS-TGFbRecd-TIM3ecd | heavy chain = SEQ ID NO: 526 |
| (GSK3359609) | light chain = SEQ ID NO: 521 |
| anti-ICOS-TGFbRecd | heavy chain = SEQ ID NO: 526 |
| (GSK3359609) | light chain = SEQ ID NO: 59 |
| anti-ICOS-TIM3ecd-BTLAecd | heavy chain = SEQ ID NO: 527 |
| (GSK3359609) | light chain = SEQ ID NO: 523 |
| anti-ICOS-TIM3ecd-PD1ecd | heavy chain = SEQ ID NO: 527 |
| (GSK3359609) | light chain = SEQ ID NO: 518 |
| anti-ICOS-TIM3ecd-SIGLEC10ecd | heavy chain = SEQ ID NO: 527 |
| (GSK3359609) | light chain = SEQ ID NO: 519 |
| anti-ICOS-TIM3ecd-SIRPaecd | heavy chain = SEQ ID NO: 527 |
| (GSK3359609) | light chain = SEQ ID NO: 520 |
| anti-ICOS-TIM3ecd | heavy chain = SEQ ID NO: 527 |
| (GSK3359609) | light chain = SEQ ID NO: 59 |
| anti-ICOS-VEGFRecd-BTLAecd | heavy chain = SEQ ID NO: 528 |
| (GSK3359609) | light chain = SEQ ID NO: 523 |
| anti-ICOS-VEGFRecd-PD1ecd | heavy chain = SEQ ID NO: 528 |
| (GSK3359609) | light chain = SEQ ID NO: 518 |
| anti-ICOS-VEGFRecd-SIGLEC10ecd | heavy chain = SEQ ID NO: 528 |
| (GSK3359609) | light chain = SEQ ID NO: 519 |
| anti-ICOS-VEGFRecd-SIRPaecd | heavy chain = SEQ ID NO: 528 |
| (GSK3359609) | light chain = SEQ ID NO: 520 |
| anti-ICOS-VEGFRecd-TIM3ecd | heavy chain = SEQ ID NO: 528 |
| (GSK3359609) | light chain = SEQ ID NO: 521 |
| anti-ICOS-VEGFRecd | heavy chain = SEQ ID NO: 528 |
| (GSK3359609) | light chain = SEQ ID NO: 59 |
| BTLAecd-Fc-PD1ecd | SEQ ID NO: 529 |
| BTLAecd-Fc-SIGLEC10ecd | SEQ ID NO: 530 |
| BTLAecd-Fc-SIRPaecd | SEQ ID NO: 531 |
| BTLAecd-Fc-TGFbRecd | SEQ ID NO: 532 |
| BTLAecd-Fc-TIM3ecd | SEQ ID NO: 533 |
| BTLAecd-Fc-VEGFRecd | SEQ ID NO: 534 |
| PD1ecd-Fc-BTLAecd | SEQ ID NO: 535 |
| PD1ecd-Fc-SIGLEC10ecd | SEQ ID NO: 536 |
| PD1ecd-Fc-SIRPaecd | SEQ ID NO: 537 |
| PD1ecd-Fc-TGFbRecd | SEQ ID NO: 538 |
| PD1ecd-Fc-TIM3ecd | SEQ ID NO: 539 |
| PD1ecd-Fc-VEGFRecd | SEQ ID NO: 540 |
| SIGLEC10ecd-Fc-BTLAecd | SEQ ID NO: 541 |
| SIGLEC10ecd-Fc-PD1ecd | SEQ ID NO: 542 |
| SIGLEC10ecd-Fc-SIRPaecd | SEQ ID NO: 543 |
| SIGLEC10ecd-Fc-TGFbRecd | SEQ ID NO: 544 |
| SIGLEC10ecd-Fc-TIM3ecd | SEQ ID NO: 545 |
| SIGLEC10ecd-Fc-VEGFRecd | SEQ ID NO: 546 |
| SIRPaecd-Fc-BTLAecd | SEQ ID NO: 547 |
| SIRPaecd-Fc-PD1ecd | SEQ ID NO: 548 |
| SIRPaecd-Fc-SIGLEC10ecd | SEQ ID NO: 549 |
| SIRPaecd-Fc-TGFbRecd | SEQ ID NO: 550 |
| SIRPaecd-Fc-TIM3ecd | SEQ ID NO: 551 |
| SIRPaecd-Fc-VEGFRecd | SEQ ID NO: 552 |
| TGFbRecd-Fc-BTLAecd | SEQ ID NO: 553 |
| TGFbRecd-Fc-PD1ecd | SEQ ID NO: 554 |
| TGFbRecd-Fc-SIGLEC10ecd | SEQ ID NO: 555 |
| TGFbRecd-Fc-SIRPaecd | SEQ ID NO: 556 |
| TGFbRecd-Fc-TIM3ecd | SEQ ID NO: 557 |
| TGFbRecd-Fc-VEGFRecd | SEQ ID NO: 558 |
| TIM3ecd-Fc-BTLAecd | SEQ ID NO: 559 |
| TIM3ecd-Fc-PD1ecd | SEQ ID NO: 560 |
| TIM3ecd-Fc-SIGLEC10ecd | SEQ ID NO: 561 |
| TIM3ecd-Fc-SIRPaecd | SEQ ID NO: 562 |
| TIM3ecd-Fc-TGFbRecd | SEQ ID NO: 563 |
| TIM3ecd-Fc-VEGFRecd | SEQ ID NO: 564 |
| VEGFRecd-Fc-BTLAecd | SEQ ID NO: 565 |
| VEGFRecd-Fc-PD1ecd | SEQ ID NO: 566 |
| VEGFRecd-Fc-SIGLEC10ecd | SEQ ID NO: 567 |
| VEGFRecd-Fc-SIRPaecd | SEQ ID NO: 568 |
| VEGFRecd-Fc-TGFbRecd | SEQ ID NO: 569 |
| VEGFRecd-Fc-TIM3ecd | SEQ ID NO: 570 |
| BTLAecd-Fc-IL12ecd | SEQ ID NO: 571 |
| IL12ecd-Fc-BTLAecd | SEQ ID NO: 572 |
| BTLAecd-Fc-IL15ecd | SEQ ID NO: 573 |
| IL15ecd-Fc-BTLAecd | SEQ ID NO: 574 |
| PD1ecd-Fc-IL12ecd | SEQ ID NO: 575 |
| IL12ecd-Fc-PD1ecd | SEQ ID NO: 576 |
| PD1ecd-Fc-IL15ecd | SEQ ID NO: 577 |
| IL15ecd-Fc-PD1ecd | SEQ ID NO: 578 |
| SIGLEC10ecd-Fc-IL12ecd | SEQ ID NO: 579 |
| IL12ecd-Fc-SIGLEC10ecd | SEQ ID NO: 580 |
| SIGLEC10ecd-Fc-IL15ecd | SEQ ID NO: 581 |
| IL15ecd-Fc-SIGLEC10ecd | SEQ ID NO: 582 |
| SIRPaecd-Fc-IL12ecd | SEQ ID NO: 583 |
| IL12ecd-Fc-SIRPaecd | SEQ ID NO: 584 |
| SIRPaecd-Fc-IL15ecd | SEQ ID NO: 585 |
| IL15ecd-Fc-SIRPaecd | SEQ ID NO: 586 |
| TGFbRecd-Fc-IL12ecd | SEQ ID NO: 587 |
| IL12ecd-Fc-TGFbRecd | SEQ ID NO: 588 |
| TGFbRecd-Fc-IL15ecd | SEQ ID NO: 589 |
| IL15ecd-Fc-TGFbRecd | SEQ ID NO: 590 |
| TIM3ecd-Fc-IL12ecd | SEQ ID NO: 591 |
| IL12ecd-Fc-TIM3ecd | SEQ ID NO: 592 |
| TIM3ecd-Fc-IL15ecd | SEQ ID NO: 593 |
| IL15ecd-Fc-TIM3ecd | SEQ ID NO: 594 |
| VEGFRecd-Fc-IL12ecd | SEQ ID NO: 595 |
| IL12ecd-Fc-VEGFRecd | SEQ ID NO: 596 |
| VEGFRecd-Fc-IL15ecd | SEQ ID NO: 597 |
| IL15ecd-Fc-VEGFRecd | SEQ ID NO: 598 |
| SIGLEC10ecd-Fc-41BBLecd | SEQ ID NO: 599 |
| 41BBLecd-Fc-SIGLEC10ecd | SEQ ID NO: 600 |
| SIGLEC10ecd-Fc-CD30Lecd | SEQ ID NO: 601 |
| CD30Lecd-Fc-SIGLEC10ecd | SEQ ID NO: 602 |
| SIGLEC10ecd-Fc-CD40Lecd | SEQ ID NO: 603 |
| CD40Lecd-Fc-SIGLEC10ecd | SEQ ID NO: 604 |
| SIGLEC10ecd-Fc-CD70ecd | SEQ ID NO: 605 |
| CD70ecd-Fc-SIGLEC10ecd | SEQ ID NO: 606 |
| SIGLEC10ecd-Fc-GITRLecd | SEQ ID NO: 607 |
| GITRLecd-Fc-SIGLEC10ecd | SEQ ID NO: 608 |
| SIGLEC10ecd-Fc-ICOSLecd | SEQ ID NO: 609 |
| ICOSLecd-Fc-SIGLEC10ecd | SEQ ID NO: 610 |
| SIGLEC10ecd-Fc-IL12ecd | SEQ ID NO: 579 |
| IL12ecd-Fc-SIGLEC10ecd | SEQ ID NO: 580 |
| SIGLEC10ecd-Fc-IL15ecd | SEQ ID NO: 581 |
| IL15ecd-Fc-SIGLEC10ecd | SEQ ID NO: 582 |
| SIGLEC10ecd-Fc-LIGHTecd | SEQ ID NO: 611 |
| LIGHTecd-Fc-SIGLEC10ecd | SEQ ID NO: 612 |
| SIGLEC10ecd-Fc-OX40Lecd | SEQ ID NO: 613 |
| OX40Lecd-Fc-SIGLEC10ecd | SEQ ID NO: 614 |
| TGFbRecd-Fc-41BBLecd | SEQ ID NO: 615 |
| 41BBLecd-Fc-TGFbRecd | SEQ ID NO: 616 |
| TGFbRecd-Fc-CD30Lecd | SEQ ID NO: 617 |
| CD30Lecd-Fc-TGFbRecd | SEQ ID NO: 618 |
| TGFbRecd-Fc-CD40Lecd | SEQ ID NO: 619 |
| CD40Lecd-Fc-TGFbRecd | SEQ ID NO: 620 |
| TGFbRecd-Fc-CD70ecd | SEQ ID NO: 621 |
| CD70ecd-Fc-TGFbRecd | SEQ ID NO: 622 |
| TGFbRecd-Fc-GITRLecd | SEQ ID NO: 623 |
| GITRLecd-Fc-TGFbRecd | SEQ ID NO: 624 |
| TGFbRecd-Fc-ICOSLecd | SEQ ID NO: 625 |
| ICOSLecd-Fc-TGFbRecd | SEQ ID NO: 626 |
| TGFbRecd-Fc-IL12ecd | SEQ ID NO: 587 |
| IL12ecd-Fc-TGFbRecd | SEQ ID NO: 588 |
| TGFbRecd-Fc-IL15ecd | SEQ ID NO: 589 |
| IL15ecd-Fc-TGFbRecd | SEQ ID NO: 590 |
| TGFbRecd-Fc-LIGHTecd | SEQ ID NO: 627 |
| LIGHTecd-Fc-TGFbRecd | SEQ ID NO: 628 |
| TGFbRecd-Fc-OX40Lecd | SEQ ID NO: 629 |
| OX40Lecd-Fc-TGFbRecd | SEQ ID NO: 630 |
| VEGFRecd-Fc-41BBLecd | SEQ ID NO: 631 |
| 41BBLecd-Fc-VEGFRecd | SEQ ID NO: 632 |
| VEGFRecd-Fc-CD30Lecd | SEQ ID NO: 633 |
| CD30Lecd-Fc-VEGFRecd | SEQ ID NO: 634 |
| VEGFRecd-Fc-CD40Lecd | SEQ ID NO: 635 |
| CD40Lecd-Fc-VEGFRecd | SEQ ID NO: 636 |
| VEGFRecd-Fc-CD70ecd | SEQ ID NO: 637 |
| CD70ecd-Fc-VEGFRecd | SEQ ID NO: 638 |
| VEGFRecd-Fc-GITRLecd | SEQ ID NO: 639 |
| GITRLecd-Fc-VEGFRecd | SEQ ID NO: 640 |
| VEGFRecd-Fc-ICOSLecd | SEQ ID NO: 641 |
| ICOSLecd-Fc-VEGFRecd | SEQ ID NO: 642 |
| VEGFRecd-Fc-IL12ecd | SEQ ID NO: 595 |
| IL12ecd-Fc-VEGFRecd | SEQ ID NO: 596 |
| VEGFRecd-Fc-IL15ecd | SEQ ID NO: 597 |
| IL15ecd-Fc-VEGFRecd | SEQ ID NO: 598 |
| VEGFRecd-Fc-LIGHTecd | SEQ ID NO: 643 |
| LIGHTecd-Fc-VEGFRecd | SEQ ID NO: 644 |
| VEGFRecd-Fc-OX40Lecd | SEQ ID NO: 645 |
| OX40Lecd-Fc-VEGFRecd | SEQ ID NO: 646 |
| anti-PDL1-41BBLecd | heavy chain = SEQ ID NO: 647 |
| (atezolizumab) | light chain = SEQ ID NO: 109 |
| anti-PDL1-41BBLecd-BTLAecd | heavy chain = SEQ ID NO: 647 |
| (atezolizumab) | light chain = SEQ ID NO: 464 |
| anti-PDL1-41BBLecd-SIGLEC10ecd | heavy chain = SEQ ID NO: 647 |
| (atezolizumab) | light chain = SEQ ID NO: 460 |
| anti-PDL1-41BBLecd-SIRPaecd | heavy chain = SEQ ID NO: 647 |
| (atezolizumab) | light chain = SEQ ID NO: 461 |
| anti-PDL1-41BBLecd-TIM3ecd | heavy chain = SEQ ID NO: 647 |
| (atezolizumab) | light chain = SEQ ID NO: 462 |
| anti-PDL1-CD30Lecd | heavy chain = SEQ ID NO: 648 |
| (atezolizumab) | light chain = SEQ ID NO: 109 |
| anti-PDL1-CD30Lecd-BTLAecd | heavy chain = SEQ ID NO: 648 |
| (atezolizumab) | light chain = SEQ ID NO: 464 |
| anti-PDL1-CD30Lecd-SIGLEC10ecd | heavy chain = SEQ ID NO: 648 |
| (atezolizumab) | light chain = SEQ ID NO: 460 |
| anti-PDL1-CD30Lecd-SIRPaecd | heavy chain = SEQ ID NO: 648 |
| (atezolizumab) | light chain = SEQ ID NO: 461 |
| anti-PDL1-CD30Lecd-TIM3ecd | heavy chain = SEQ ID NO: 648 |
| (atezolizumab) | light chain = SEQ ID NO: 462 |
| anti-PDL1-CD40Lecd | heavy chain = SEQ ID NO: 649 |
| (atezolizumab) | light chain = SEQ ID NO: 109 |
| anti-PDL1-CD40Lecd-BTLAecd | heavy chain = SEQ ID NO: 649 |
| (atezolizumab) | light chain = SEQ ID NO: 464 |
| anti-PDL1-CD40Lecd-SIGLEC10ecd | heavy chain = SEQ ID NO: 649 |
| (atezolizumab) | light chain = SEQ ID NO: 460 |
| anti-PDL1-CD40Lecd-SIRPaecd | heavy chain = SEQ ID NO: 649 |
| (atezolizumab) | light chain = SEQ ID NO: 461 |
| anti-PDL1-CD40Lecd-TIM3ecd | heavy chain = SEQ ID NO: 649 |
| (atezolizumab) | light chain = SEQ ID NO: 462 |
| anti-PDL1-CD70ecd | heavy chain = SEQ ID NO: 650 |
| (atezolizumab) | light chain = SEQ ID NO: 109 |
| anti-PDL1-CD70ecd-BTLAecd | heavy chain = SEQ ID NO: 650 |
| (atezolizumab) | light chain = SEQ ID NO: 464 |
| anti-PDL1-CD70ecd-SIGLEC10ecd | heavy chain = SEQ ID NO: 650 |
| (atezolizumab) | light chain = SEQ ID NO: 460 |
| anti-PDL1-CD70ecd-SIRPaecd | heavy chain = SEQ ID NO: 650 |
| (atezolizumab) | light chain = SEQ ID NO: 461 |
| anti-PDL1-CD70ecd-TIM3ecd | heavy chain = SEQ ID NO: 650 |
| (atezolizumab) | light chain = SEQ ID NO: 462 |
| anti-PDL1-GITRLecd | heavy chain = SEQ ID NO: 651 |
| (atezolizumab) | light chain = SEQ ID NO: 109 |
| anti-PDL1-GITRLecd-BTLAecd | heavy chain = SEQ ID NO: 651 |
| (atezolizumab) | light chain = SEQ ID NO: 464 |
| anti-PDL1-GITRLecd-SIGLEC10ecd | heavy chain = SEQ ID NO: 651 |
| (atezolizumab) | light chain = SEQ ID NO: 460 |
| anti-PDL1-GITRLecd-SIRPaecd | heavy chain = SEQ ID NO: 651 |
| (atezolizumab) | light chain = SEQ ID NO: 461 |
| anti-PDL1-GITRLecd-TIM3ecd | heavy chain = SEQ ID NO: 651 |
| (atezolizumab) | light chain = SEQ ID NO: 462 |
| anti-PDL1-ICOSLecd | heavy chain = SEQ ID NO: 652 |
| (atezolizumab) | light chain = SEQ ID NO: 109 |
| anti-PDL1-ICOSLecd-BTLAecd | heavy chain = SEQ ID NO: 652 |
| (atezolizumab) | light chain = SEQ ID NO: 464 |
| anti-PDL1-ICOSLecd-SIGLEC10ecd | heavy chain = SEQ ID NO: 652 |
| (atezolizumab) | light chain = SEQ ID NO: 460 |
| anti-PDL1-ICOSLecd-SIRPaecd | heavy chain = SEQ ID NO: 652 |
| (atezolizumab) | light chain = SEQ ID NO: 461 |
| anti-PDL1-ICOSLecd-TIM3ecd | heavy chain = SEQ ID NO: 652 |
| (atezolizumab) | light chain = SEQ ID NO: 462 |
| anti-PDL1-IL12ecd | heavy chain = SEQ ID NO: 653 |
| (atezolizumab) | light chain = SEQ ID NO: 109 |
| anti-PDL1-IL12ecd-BTLAecd | heavy chain = SEQ ID NO: 653 |
| (atezolizumab) | light chain = SEQ ID NO: 464 |
| anti-PDL1-IL12ecd-SIGLEC10ecd | heavy chain = SEQ ID NO: 653 |
| (atezolizumab) | light chain = SEQ ID NO: 460 |
| anti-PDL1-IL12ecd-SIRPaecd | heavy chain = SEQ ID NO: 653 |
| (atezolizumab) | light chain = SEQ ID NO: 461 |
| anti-PDL1-IL12ecd-TIM3ecd | heavy chain = SEQ ID NO: 653 |
| (atezolizumab) | light chain = SEQ ID NO: 462 |
| anti-PDL1-IL15ecd | heavy chain = SEQ ID NO: 654 |
| (atezolizumab) | light chain = SEQ ID NO: 109 |
| anti-PDL1-IL15ecd-BTLAecd | heavy chain = SEQ ID NO: 654 |
| (atezolizumab) | light chain = SEQ ID NO: 464 |
| anti-PDL1-IL15ecd-SIGLEC10ecd | heavy chain = SEQ ID NO: 654 |
| (atezolizumab) | light chain = SEQ ID NO: 460 |
| anti-PDL1-IL15ecd-SIRPaecd | heavy chain = SEQ ID NO: 654 |
| (atezolizumab) | light chain = SEQ ID NO: 461 |
| anti-PDL1-IL15ecd-TIM3ecd | heavy chain = SEQ ID NO: 654 |
| (atezolizumab) | light chain = SEQ ID NO: 462 |
| anti-PDL1-LIGHTecd | heavy chain = SEQ ID NO: 655 |
| (atezolizumab) | light chain = SEQ ID NO: 109 |
| anti-PDL1-LIGHTecd-BTLAecd | heavy chain = SEQ ID NO: 655 |
| (atezolizumab) | light chain = SEQ ID NO: 464 |
| anti-PDL1-LIGHTecd-SIGLEC10ecd | heavy chain = SEQ ID NO: 655 |
| (atezolizumab) | light chain = SEQ ID NO: 460 |
| anti-PDL1-LIGHTecd-SIRPaecd | heavy chain = SEQ ID NO: 655 |
| (atezolizumab) | light chain = SEQ ID NO: 461 |
| anti-PDL1-LIGHTecd-TIM3ecd | heavy chain = SEQ ID NO: 655 |
| (atezolizumab) | light chain = SEQ ID NO: 462 |
| anti-PDL1-OX40Lecd | heavy chain = SEQ ID NO: 656 |
| (atezolizumab) | light chain = SEQ ID NO: 109 |
| anti-PDL1-OX40Lecd-BTLAecd | heavy chain = SEQ ID NO: 656 |
| (atezolizumab) | light chain = SEQ ID NO: 464 |
| anti-PDL1-OX40Lecd-SIGLEC10ecd | heavy chain = SEQ ID NO: 656 |
| (atezolizumab) | light chain = SEQ ID NO: 460 |
| anti-PDL1-OX40Lecd-SIRPaecd | heavy chain = SEQ ID NO: 656 |
| (atezolizumab) | light chain = SEQ ID NO: 461 |
| anti-PDL1-OX40Lecd-TIM3ecd | heavy chain = SEQ ID NO: 656 |
| (atezolizumab) | light chain = SEQ ID NO: 462 |
| anti-PDL1-BTLAecd-41BBLecd | heavy chain = SEQ ID NO: 459 |
| (atezolizumab) | light chain = SEQ ID NO: 657 |
| anti-PDL1-BTLAecd-CD30Lecd | heavy chain = SEQ ID NO: 459 |
| (atezolizumab) | light chain = SEQ ID NO: 658 |
| anti-PDL1-BTLAecd-CD40Lecd | heavy chain = SEQ ID NO: 459 |
| (atezolizumab) | light chain = SEQ ID NO: 659 |
| anti-PDL1-BTLAecd-CD70ecd | heavy chain = SEQ ID NO: 459 |
| (atezolizumab) | light chain = SEQ ID NO: 660 |
| anti-PDL1-BTLAecd-GITRLecd | heavy chain = SEQ ID NO: 459 |
| (atezolizumab) | light chain = SEQ ID NO: 661 |
| anti-PDL1-BTLAecd-ICOSLecd | heavy chain = SEQ ID NO: 459 |
| (atezolizumab) | light chain = SEQ ID NO: 662 |
| anti-PDL1-BTLAecd-IL12ecd | heavy chain = SEQ ID NO: 459 |
| (atezolizumab) | light chain = SEQ ID NO: 663 |
| anti-PDL1-BTLAecd-IL15ecd | heavy chain = SEQ ID NO: 459 |
| (atezolizumab) | light chain = SEQ ID NO: 664 |
| anti-PDL1-BTLAecd-LIGHTecd | heavy chain = SEQ ID NO: 459 |
| (atezolizumab) | light chain = SEQ ID NO: 665 |
| anti-PDL1-BTLAecd-OX40Lecd | heavy chain = SEQ ID NO: 459 |
| (atezolizumab) | light chain = SEQ ID NO: 666 |
| anti-PDL1-SIGLEC10ecd-41BBLecd | heavy chain = SEQ ID NO: 463 |
| (atezolizumab) | light chain = SEQ ID NO: 657 |
| anti-PDL1-SIGLEC10ecd-CD30Lecd | heavy chain = SEQ ID NO: 463 |
| (atezolizumab) | light chain = SEQ ID NO: 658 |
| anti-PDL1-SIGLEC10ecd-CD40Lecd | heavy chain = SEQ ID NO: 463 |
| (atezolizumab) | light chain = SEQ ID NO: 659 |
| anti-PDL1-SIGLEC10ecd-CD70ecd | heavy chain = SEQ ID NO: 463 |
| (atezolizumab) | light chain = SEQ ID NO: 660 |
| anti-PDL1-SIGLEC10ecd-GITRLecd | heavy chain = SEQ ID NO: 463 |
| (atezolizumab) | light chain = SEQ ID NO: 661 |
| anti-PDL1-SIGLEC10ecd-ICOSLecd | heavy chain = SEQ ID NO: 463 |
| (atezolizumab) | light chain = SEQ ID NO: 662 |
| anti-PDL1-SIGLEC10ecd-IL12ecd | heavy chain = SEQ ID NO: 463 |
| (atezolizumab) | light chain = SEQ ID NO: 663 |
| anti-PDL1-SIGLEC10ecd-IL15ecd | heavy chain = SEQ ID NO: 463 |
| (atezolizumab) | light chain = SEQ ID NO: 664 |
| anti-PDL1-SIGLEC10ecd-LIGHTecd | heavy chain = SEQ ID NO: 463 |
| (atezolizumab) | light chain = SEQ ID NO: 665 |
| anti-PDL1-SIGLEC10ecd-OX40Lecd | heavy chain = SEQ ID NO: 463 |
| (atezolizumab) | light chain = SEQ ID NO: 666 |
| anti-PDL1-SIRPaecd-41BBLecd | heavy chain = SEQ ID NO: 465 |
| (atezolizumab) | light chain = SEQ ID NO: 657 |
| anti-PDL1-SIRPaecd-CD30Lecd | heavy chain = SEQ ID NO: 465 |
| (atezolizumab) | light chain = SEQ ID NO: 658 |
| anti-PDL1-SIRPaecd-CD40Lecd | heavy chain = SEQ ID NO: 465 |
| (atezolizumab) | light chain = SEQ ID NO: 659 |
| anti-PDL1-SIRPaecd-CD70ecd | heavy chain = SEQ ID NO: 465 |
| (atezolizumab) | light chain = SEQ ID NO: 660 |
| anti-PDL1-SIRPaecd-GITRLecd | heavy chain = SEQ ID NO: 465 |
| (atezolizumab) | light chain = SEQ ID NO: 661 |
| anti-PDL1-SIRPaecd-ICOSLecd | heavy chain = SEQ ID NO: 465 |
| (atezolizumab) | light chain = SEQ ID NO: 662 |
| anti-PDL1-SIRPaecd-IL12ecd | heavy chain = SEQ ID NO: 465 |
| (atezolizumab) | light chain = SEQ ID NO: 663 |
| anti-PDL1-SIRPaecd-IL15ecd | heavy chain = SEQ ID NO: 465 |
| (atezolizumab) | light chain = SEQ ID NO: 664 |
| anti-PDL1-SIRPaecd-LIGHTecd | heavy chain = SEQ ID NO: 465 |
| (atezolizumab) | light chain = SEQ ID NO: 665 |
| anti-PDL1-SIRPaecd-OX40Lecd | heavy chain = SEQ ID NO: 465 |
| (atezolizumab) | light chain = SEQ ID NO: 666 |
| anti-PDL1-TGFbRecd-41BBLecd | heavy chain = SEQ ID NO: 466 |
| (atezolizumab) | light chain = SEQ ID NO: 657 |
| anti-PDL1-TGFbRecd-CD30Lecd | heavy chain = SEQ ID NO: 466 |
| (atezolizumab) | light chain = SEQ ID NO: 658 |
| anti-PDL1-TGFbRecd-CD40Lecd | heavy chain = SEQ ID NO: 466 |
| (atezolizumab) | light chain = SEQ ID NO: 659 |
| anti-PDL1-TGFbRecd-CD70ecd | heavy chain = SEQ ID NO: 466 |
| (atezolizumab) | light chain = SEQ ID NO: 660 |
| anti-PDL1-TGFbRecd-GITRLecd | heavy chain = SEQ ID NO: 466 |
| (atezolizumab) | light chain = SEQ ID NO: 661 |
| anti-PDL1-TGFbRecd-ICOSLecd | heavy chain = SEQ ID NO: 466 |
| (atezolizumab) | light chain = SEQ ID NO: 662 |
| anti-PDL1-TGFbRecd-IL12ecd | heavy chain = SEQ ID NO: 466 |
| (atezolizumab) | light chain = SEQ ID NO: 663 |
| anti-PDL1-TGFbRecd-IL15ecd | heavy chain = SEQ ID NO: 466 |
| (atezolizumab) | light chain = SEQ ID NO: 664 |
| anti-PDL1-TGFbRecd-LIGHTecd | heavy chain = SEQ ID NO: 466 |
| (atezolizumab) | light chain = SEQ ID NO: 665 |
| anti-PDL1-TGFbRecd-OX40Lecd | heavy chain = SEQ ID NO: 466 |
| (atezolizumab) | light chain = SEQ ID NO: 666 |
| anti-PDL1-TIM3ecd-41BBLecd | heavy chain = SEQ ID NO: 467 |
| (atezolizumab) | light chain = SEQ ID NO: 657 |
| anti-PDL1-TIM3ecd-CD30Lecd | heavy chain = SEQ ID NO: 467 |
| (atezolizumab) | light chain = SEQ ID NO: 658 |
| anti-PDL1-TIM3ecd-CD40Lecd | heavy chain = SEQ ID NO: 467 |
| (atezolizumab) | light chain = SEQ ID NO: 659 |
| anti-PDL1-TIM3ecd-CD70ecd | heavy chain = SEQ ID NO: 467 |
| (atezolizumab) | light chain = SEQ ID NO: 660 |
| anti-PDL1-TIM3ecd-GITRLecd | heavy chain = SEQ ID NO: 467 |
| (atezolizumab) | light chain = SEQ ID NO: 661 |
| anti-PDL1-TIM3ecd-ICOSLecd | heavy chain = SEQ ID NO: 467 |
| (atezolizumab) | light chain = SEQ ID NO: 662 |
| anti-PDL1-TIM3ecd-IL12ecd | heavy chain = SEQ ID NO: 467 |
| (atezolizumab) | light chain = SEQ ID NO: 663 |
| anti-PDL1-TIM3ecd-IL15ecd | heavy chain = SEQ ID NO: 467 |
| (atezolizumab) | light chain = SEQ ID NO: 664 |
| anti-PDL1-TIM3ecd-LIGHTecd | heavy chain = SEQ ID NO: 467 |
| (atezolizumab) | light chain = SEQ ID NO: 665 |
| anti-PDL1-TIM3ecd-OX40Lecd | heavy chain = SEQ ID NO: 467 |
| (atezolizumab) | light chain = SEQ ID NO: 666 |
| anti-PDL1-VEGFRecd-41BBLecd | heavy chain = SEQ ID NO: 468 |
| (atezolizumab) | light chain = SEQ ID NO: 657 |
| anti-PDL1-VEGFRecd-CD30Lecd | heavy chain = SEQ ID NO: 468 |
| (atezolizumab) | light chain = SEQ ID NO: 658 |
| anti-PDL1-VEGFRecd-CD40Lecd | heavy chain = SEQ ID NO: 468 |
| (atezolizumab) | light chain = SEQ ID NO: 659 |
| anti-PDL1-VEGFRecd-CD70ecd | heavy chain = SEQ ID NO: 468 |
| (atezolizumab) | light chain = SEQ ID NO: 660 |
| anti-PDL1-VEGFRecd-GITRLecd | heavy chain = SEQ ID NO: 468 |
| (atezolizumab) | light chain = SEQ ID NO: 661 |
| anti-PDL1-VEGFRecd-ICOSLecd | heavy chain = SEQ ID NO: 468 |
| (atezolizumab) | light chain = SEQ ID NO: 662 |
| anti-PDL1-VEGFRecd-IL12ecd | heavy chain = SEQ ID NO: 468 |
| (atezolizumab) | light chain = SEQ ID NO: 663 |
| anti-PDL1-VEGFRecd-IL15ecd | heavy chain = SEQ ID NO: 468 |
| (atezolizumab) | light chain = SEQ ID NO: 664 |
| anti-PDL1-VEGFRecd-LIGHTecd | heavy chain = SEQ ID NO: 468 |
| (atezolizumab) | light chain = SEQ ID NO: 665 |
| anti-PDL1-VEGFRecd-OX40Lecd | heavy chain = SEQ ID NO: 468 |
| (atezolizumab) | light chain = SEQ ID NO: 666 |
| anti-EGFR-41BBLecd | heavy chain = SEQ ID NO: 667 |
| (cetuximab) | light chain = SEQ ID NO: 43 |
| anti-EGFR-41BBLecd-BTLAecd | heavy chain = SEQ ID NO: 667 |
| (cetuximab) | light chain = SEQ ID NO: 226 |
| anti-EGFR-41BBLecd-PD1ecd | heavy chain = SEQ ID NO: 667 |
| (cetuximab) | light chain = SEQ ID NO: 221 |
| anti-EGFR-41BBLecd-SIGLEC10ecd | heavy chain = SEQ ID NO: 667 |
| (cetuximab) | light chain = SEQ ID NO: 222 |
| anti-EGFR-41BBLecd-SIRPaecd | heavy chain = SEQ ID NO: 667 |
| (cetuximab) | light chain = SEQ ID NO: 223 |
| anti-EGFR-41BBLecd-TIM3ecd | heavy chain = SEQ ID NO: 667 |
| (cetuximab) | light chain = SEQ ID NO: 224 |
| anti-EGFR-CD30Lecd | heavy chain = SEQ ID NO: 668 |
| (cetuximab) | light chain = SEQ ID NO: 43 |
| anti-EGFR-CD30Lecd-BTLAecd | heavy chain = SEQ ID NO: 668 |
| (cetuximab) | light chain = SEQ ID NO: 226 |
| anti-EGFR-CD30Lecd-PD1ecd | heavy chain = SEQ ID NO: 668 |
| (cetuximab) | light chain = SEQ ID NO: 221 |
| anti-EGFR-CD30Lecd-SIGLEC10ecd | heavy chain = SEQ ID NO: 668 |
| (cetuximab) | light chain = SEQ ID NO: 222 |
| anti-EGFR-CD30Lecd-SIRPaecd | heavy chain = SEQ ID NO: 668 |
| (cetuximab) | light chain = SEQ ID NO: 223 |
| anti-EGFR-CD30Lecd-TIM3ecd | heavy chain = SEQ ID NO: 668 |
| (cetuximab) | light chain = SEQ ID NO: 224 |
| anti-EGFR-CD40Lecd | heavy chain = SEQ ID NO: 669 |
| (cetuximab) | light chain = SEQ ID NO: 43 |
| anti-EGFR-CD40Lecd-BTLAecd | heavy chain = SEQ ID NO: 669 |
| (cetuximab) | light chain = SEQ ID NO: 226 |
| anti-EGFR-CD40Lecd-PD1ecd | heavy chain = SEQ ID NO: 669 |
| (cetuximab) | light chain = SEQ ID NO: 221 |
| anti-EGFR-CD40Lecd-SIGLEC10ecd | heavy chain = SEQ ID NO: 669 |
| (cetuximab) | light chain = SEQ ID NO: 222 |
| anti-EGFR-CD40Lecd-SIRPaecd | heavy chain = SEQ ID NO: 669 |
| (cetuximab) | light chain = SEQ ID NO: 223 |
| anti-EGFR-CD40Lecd-TIM3ecd | heavy chain = SEQ ID NO: 669 |
| (cetuximab) | light chain = SEQ ID NO: 224 |
| anti-EGFR-CD70ecd | heavy chain = SEQ ID NO: 670 |
| (cetuximab) | light chain = SEQ ID NO: 43 |
| anti-EGFR-CD70ecd-BTLAecd | heavy chain = SEQ ID NO: 670 |
| (cetuximab) | light chain = SEQ ID NO: 226 |
| anti-EGFR-CD70ecd-PD1ecd | heavy chain = SEQ ID NO: 670 |
| (cetuximab) | light chain = SEQ ID NO: 221 |
| anti-EGFR-CD70ecd-SIGLEC10ecd | heavy chain = SEQ ID NO: 670 |
| (cetuximab) | light chain = SEQ ID NO: 222 |
| anti-EGFR-CD70ecd-SIRPaecd | heavy chain = SEQ ID NO: 670 |
| (cetuximab) | light chain = SEQ ID NO: 223 |
| anti-EGFR-CD70ecd-TIM3ecd | heavy chain = SEQ ID NO: 670 |
| (cetuximab) | light chain = SEQ ID NO: 224 |
| anti-EGFR-GITRLecd | heavy chain = SEQ ID NO: 671 |
| (cetuximab) | light chain = SEQ ID NO: 43 |
| anti-EGFR-GITRLecd-BTLAecd | heavy chain = SEQ ID NO: 671 |
| (cetuximab) | light chain = SEQ ID NO: 226 |
| anti-EGFR-GITRLecd-PD1ecd | heavy chain = SEQ ID NO: 671 |
| (cetuximab) | light chain = SEQ ID NO: 221 |
| anti-EGFR-GITRLecd-SIGLEC10ecd | heavy chain = SEQ ID NO: 671 |
| (cetuximab) | light chain = SEQ ID NO: 222 |
| anti-EGFR-GITRLecd-SIRPaecd | heavy chain = SEQ ID NO: 671 |
| (cetuximab) | light chain = SEQ ID NO: 223 |
| anti-EGFR-GITRLecd-TIM3ecd | heavy chain = SEQ ID NO: 671 |
| (cetuximab) | light chain = SEQ ID NO: 224 |
| anti-EGFR-ICOSLecd | heavy chain = SEQ ID NO: 672 |
| (cetuximab) | light chain = SEQ ID NO: 43 |
| anti-EGFR-ICOSLecd-BTLAecd | heavy chain = SEQ ID NO: 672 |
| (cetuximab) | light chain = SEQ ID NO: 226 |
| anti-EGFR-ICOSLecd-PD1ecd | heavy chain = SEQ ID NO: 672 |
| (cetuximab) | light chain = SEQ ID NO: 221 |
| anti-EGFR-ICOSLecd-SIGLEC10ecd | heavy chain = SEQ ID NO: 672 |
| (cetuximab) | light chain = SEQ ID NO: 222 |
| anti-EGFR-ICOSLecd-SIRPaecd | heavy chain = SEQ ID NO: 672 |
| (cetuximab) | light chain = SEQ ID NO: 223 |
| anti-EGFR-ICOSLecd-TIM3ecd | heavy chain = SEQ ID NO: 672 |
| (cetuximab) | light chain = SEQ ID NO: 224 |
| anti-EGFR-IL12ecd | heavy chain = SEQ ID NO: 673 |
| (cetuximab) | light chain = SEQ ID NO: 43 |
| anti-EGFR-IL12ecd-BTLAecd | heavy chain = SEQ ID NO: 673 |
| (cetuximab) | light chain = SEQ ID NO: 226 |
| anti-EGFR-IL12ecd-PD1ecd | heavy chain = SEQ ID NO: 673 |
| (cetuximab) | light chain = SEQ ID NO: 221 |
| anti-EGFR-IL12ecd-SIGLEC10ecd | heavy chain = SEQ ID NO: 673 |
| (cetuximab) | light chain = SEQ ID NO: 222 |
| anti-EGFR-IL12ecd-SIRPaecd | heavy chain = SEQ ID NO: 673 |
| (cetuximab) | light chain = SEQ ID NO: 223 |
| anti-EGFR-IL12ecd-TIM3ecd | heavy chain = SEQ ID NO: 673 |
| (cetuximab) | light chain = SEQ ID NO: 224 |
| anti-EGFR-IL15ecd | heavy chain = SEQ ID NO: 674 |
| (cetuximab) | light chain = SEQ ID NO: 43 |
| anti-EGFR-IL15ecd-BTLAecd | heavy chain = SEQ ID NO: 674 |
| (cetuximab) | light chain = SEQ ID NO: 226 |
| anti-EGFR-IL15ecd-PD1ecd | heavy chain = SEQ ID NO: 674 |
| (cetuximab) | light chain = SEQ ID NO: 221 |
| anti-EGFR-IL15ecd-SIGLEC10ecd | heavy chain = SEQ ID NO: 674 |
| (cetuximab) | light chain = SEQ ID NO: 222 |
| anti-EGFR-IL15ecd-SIRPaecd | heavy chain = SEQ ID NO: 674 |
| (cetuximab) | light chain = SEQ ID NO: 223 |
| anti-EGFR-IL15ecd-TIM3ecd | heavy chain = SEQ ID NO: 674 |
| (cetuximab) | light chain = SEQ ID NO: 224 |
| anti-EGFR-LIGHTecd | heavy chain = SEQ ID NO: 675 |
| (cetuximab) | light chain = SEQ ID NO: 43 |
| anti-EGFR-LIGHTecd-BTLAecd | heavy chain = SEQ ID NO: 675 |
| (cetuximab) | light chain = SEQ ID NO: 226 |
| anti-EGFR-LIGHTecd-PD1ecd | heavy chain = SEQ ID NO: 675 |
| (cetuximab) | light chain = SEQ ID NO: 221 |
| anti-EGFR-LIGHTecd-SIGLEC10ecd | heavy chain = SEQ ID NO: 675 |
| (cetuximab) | light chain = SEQ ID NO: 222 |
| anti-EGFR-LIGHTecd-SIRPaecd | heavy chain = SEQ ID NO: 675 |
| (cetuximab) | light chain = SEQ ID NO: 223 |
| anti-EGFR-LIGHTecd-TIM3ecd | heavy chain = SEQ ID NO: 675 |
| (cetuximab) | light chain = SEQ ID NO: 224 |
| anti-EGFR-OX40Lecd | heavy chain = SEQ ID NO: 676 |
| (cetuximab) | light chain = SEQ ID NO: 43 |
| anti-EGFR-OX40Lecd-BTLAecd | heavy chain = SEQ ID NO: 676 |
| (cetuximab) | light chain = SEQ ID NO: 226 |
| anti-EGFR-OX40Lecd-PD1ecd | heavy chain = SEQ ID NO: 676 |
| (cetuximab) | light chain = SEQ ID NO: 221 |
| anti-EGFR-OX40Lecd-SIGLEC10ecd | heavy chain = SEQ ID NO: 676 |
| (cetuximab) | light chain = SEQ ID NO: 222 |
| anti-EGFR-OX40Lecd-SIRPaecd | heavy chain = SEQ ID NO: 676 |
| (cetuximab) | light chain = SEQ ID NO: 223 |
| anti-EGFR-OX40Lecd-TIM3ecd | heavy chain = SEQ ID NO: 676 |
| (cetuximab) | light chain = SEQ ID NO: 224 |
| anti-EGFR-BTLAecd-41BBLecd | heavy chain = SEQ ID NO: 220 |
| (cetuximab) | light chain = SEQ ID NO: 677 |
| anti-EGFR-BTLAecd-CD30Lecd | heavy chain = SEQ ID NO: 220 |
| (cetuximab) | light chain = SEQ ID NO: 678 |
| anti-EGFR-BTLAecd-CD40Lecd | heavy chain = SEQ ID NO: 220 |
| (cetuximab) | light chain = SEQ ID NO: 679 |
| anti-EGFR-BTLAecd-CD70ecd | heavy chain = SEQ ID NO: 220 |
| (cetuximab) | light chain = SEQ ID NO: 680 |
| anti-EGFR-BTLAecd-GITRLecd | heavy chain = SEQ ID NO: 220 |
| (cetuximab) | light chain = SEQ ID NO: 681 |
| anti-EGFR-BTLAecd-ICOSLecd | heavy chain = SEQ ID NO: 220 |
| (cetuximab) | light chain = SEQ ID NO: 682 |
| anti-EGFR-BTLAecd-IL12ecd | heavy chain = SEQ ID NO: 220 |
| (cetuximab) | light chain = SEQ ID NO: 683 |
| anti-EGFR-BTLAecd-IL15ecd | heavy chain = SEQ ID NO: 220 |
| (cetuximab) | light chain = SEQ ID NO: 684 |
| anti-EGFR-BTLAecd-LIGHTecd | heavy chain = SEQ ID NO: 220 |
| (cetuximab) | light chain = SEQ ID NO: 685 |
| anti-EGFR-BTLAecd-OX40Lecd | heavy chain = SEQ ID NO: 220 |
| (cetuximab) | light chain = SEQ ID NO: 686 |
| anti-EGFR-PD1ecd-41BBLecd | heavy chain = SEQ ID NO: 225 |
| (cetuximab) | light chain = SEQ ID NO: 677 |
| anti-EGFR-PD1ecd-CD30Lecd | heavy chain = SEQ ID NO: 225 |
| (cetuximab) | light chain = SEQ ID NO: 678 |
| anti-EGFR-PD1ecd-CD40Lecd | heavy chain = SEQ ID NO: 225 |
| (cetuximab) | light chain = SEQ ID NO: 679 |
| anti-EGFR-PD1ecd-CD70ecd | heavy chain = SEQ ID NO: 225 |
| (cetuximab) | light chain = SEQ ID NO: 680 |
| anti-EGFR-PD1ecd-GITRLecd | heavy chain = SEQ ID NO: 225 |
| (cetuximab) | light chain = SEQ ID NO: 681 |
| anti-EGFR-PD1ecd-ICOSLecd | heavy chain = SEQ ID NO: 225 |
| (cetuximab) | light chain = SEQ ID NO: 682 |
| anti-EGFR-PD1ecd-IL12ecd | heavy chain = SEQ ID NO: 225 |
| (cetuximab) | light chain = SEQ ID NO: 683 |
| anti-EGFR-PD1ecd-IL15ecd | heavy chain = SEQ ID NO: 225 |
| (cetuximab) | light chain = SEQ ID NO: 684 |
| anti-EGFR-PD1ecd-LIGHTecd | heavy chain = SEQ ID NO: 225 |
| (cetuximab) | light chain = SEQ ID NO: 685 |
| anti-EGFR-PD1ecd-OX40Lecd | heavy chain = SEQ ID NO: 225 |
| (cetuximab) | light chain = SEQ ID NO: 686 |
| anti-EGFR-SIGLEC10ecd-41BBLecd | heavy chain = SEQ ID NO: 227 |
| (cetuximab) | light chain = SEQ ID NO: 677 |
| anti-EGFR-SIGLEC10ecd-CD30Lecd | heavy chain = SEQ ID NO: 227 |
| (cetuximab) | light chain = SEQ ID NO: 678 |
| anti-EGFR-SIGLEC10ecd-CD40Lecd | heavy chain = SEQ ID NO: 227 |
| (cetuximab) | light chain = SEQ ID NO: 679 |
| anti-EGFR-SIGLEC10ecd-CD70ecd | heavy chain = SEQ ID NO: 227 |
| (cetuximab) | light chain = SEQ ID NO: 680 |
| anti-EGFR-SIGLEC10ecd-GITRLecd | heavy chain = SEQ ID NO: 227 |
| (cetuximab) | light chain = SEQ ID NO: 681 |
| anti-EGFR-SIGLEC10ecd-ICOSLecd | heavy chain = SEQ ID NO: 227 |
| (cetuximab) | light chain = SEQ ID NO: 682 |
| anti-EGFR-SIGLEC10ecd-IL12ecd | heavy chain = SEQ ID NO: 227 |
| (cetuximab) | light chain = SEQ ID NO: 683 |
| anti-EGFR-SIGLEC10ecd-IL15ecd | heavy chain = SEQ ID NO: 227 |
| (cetuximab) | light chain = SEQ ID NO: 684 |
| anti-EGFR-SIGLEC10ecd-LIGHTecd | heavy chain = SEQ ID NO: 227 |
| (cetuximab) | light chain = SEQ ID NO: 685 |
| anti-EGFR-SIGLEC10ecd-OX40Lecd | heavy chain = SEQ ID NO: 227 |
| (cetuximab) | light chain = SEQ ID NO: 686 |
| anti-EGFR-SIRPaecd-41BBLecd | heavy chain = SEQ ID NO: 228 |
| (cetuximab) | light chain = SEQ ID NO: 677 |
| anti-EGFR-SIRPaecd-CD30Lecd | heavy chain = SEQ ID NO: 228 |
| (cetuximab) | light chain = SEQ ID NO: 678 |
| anti-EGFR-SIRPaecd-CD40Lecd | heavy chain = SEQ ID NO: 228 |
| (cetuximab) | light chain = SEQ ID NO: 679 |
| anti-EGFR-SIRPaecd-CD70ecd | heavy chain = SEQ ID NO: 228 |
| (cetuximab) | light chain = SEQ ID NO: 680 |
| anti-EGFR-SIRPaecd-GITRLecd | heavy chain = SEQ ID NO: 228 |
| (cetuximab) | light chain = SEQ ID NO: 681 |
| anti-EGFR-SIRPaecd-ICOSLecd | heavy chain = SEQ ID NO: 228 |
| (cetuximab) | light chain = SEQ ID NO: 682 |
| anti-EGFR-SIRPaecd-IL12ecd | heavy chain = SEQ ID NO: 228 |
| (cetuximab) | light chain = SEQ ID NO: 683 |
| anti-EGFR-SIRPaecd-IL15ecd | heavy chain = SEQ ID NO: 228 |
| (cetuximab) | light chain = SEQ ID NO: 684 |
| anti-EGFR-SIRPaecd-LIGHTecd | heavy chain = SEQ ID NO: 228 |
| (cetuximab) | light chain = SEQ ID NO: 685 |
| anti-EGFR-SIRPaecd-OX40Lecd | heavy chain = SEQ ID NO: 228 |
| (cetuximab) | light chain = SEQ ID NO: 686 |
| anti-EGFR-TGFbRecd-41BBLecd | heavy chain = SEQ ID NO: 229 |
| (cetuximab) | light chain = SEQ ID NO: 677 |
| anti-EGFR-TGFbRecd-CD30Lecd | heavy chain = SEQ ID NO: 229 |
| (cetuximab) | light chain = SEQ ID NO: 678 |
| anti-EGFR-TGFbRecd-CD40Lecd | heavy chain = SEQ ID NO: 229 |
| (cetuximab) | light chain = SEQ ID NO: 679 |
| anti-EGFR-TGFbRecd-CD70ecd | heavy chain = SEQ ID NO: 229 |
| (cetuximab) | light chain = SEQ ID NO: 680 |
| anti-EGFR-TGFbRecd-GITRLecd | heavy chain = SEQ ID NO: 229 |
| (cetuximab) | light chain = SEQ ID NO: 681 |
| anti-EGFR-TGFbRecd-ICOSLecd | heavy chain = SEQ ID NO: 229 |
| (cetuximab) | light chain = SEQ ID NO: 682 |
| anti-EGFR-TGFbRecd-IL12ecd | heavy chain = SEQ ID NO: 229 |
| (cetuximab) | light chain = SEQ ID NO: 683 |
| anti-EGFR-TGFbRecd-IL15ecd | heavy chain = SEQ ID NO: 229 |
| (cetuximab) | light chain = SEQ ID NO: 684 |
| anti-EGFR-TGFbRecd-LIGHTecd | heavy chain = SEQ ID NO: 229 |
| (cetuximab) | light chain = SEQ ID NO: 685 |
| anti-EGFR-TGFbRecd-OX40Lecd | heavy chain = SEQ ID NO: 229 |
| (cetuximab) | light chain = SEQ ID NO: 686 |
| anti-EGFR-TIM3ecd-41BBLecd | heavy chain = SEQ ID NO: 230 |
| (cetuximab) | light chain = SEQ ID NO: 677 |
| anti-EGFR-TIM3ecd-CD30Lecd | heavy chain = SEQ ID NO: 230 |
| (cetuximab) | light chain = SEQ ID NO: 678 |
| anti-EGFR-TIM3ecd-CD40Lecd | heavy chain = SEQ ID NO: 230 |
| (cetuximab) | light chain = SEQ ID NO: 679 |
| anti-EGFR-TIM3ecd-CD70ecd | heavy chain = SEQ ID NO: 230 |
| (cetuximab) | light chain = SEQ ID NO: 680 |
| anti-EGFR-TIM3ecd-GITRLecd | heavy chain = SEQ ID NO: 230 |
| (cetuximab) | light chain = SEQ ID NO: 681 |
| anti-EGFR-TIM3ecd-ICOSLecd | heavy chain = SEQ ID NO: 230 |
| (cetuximab) | light chain = SEQ ID NO: 682 |
| anti-EGFR-TIM3ecd-IL12ecd | heavy chain = SEQ ID NO: 230 |
| (cetuximab) | light chain = SEQ ID NO: 683 |
| anti-EGFR-TIM3ecd-IL15ecd | heavy chain = SEQ ID NO: 230 |
| (cetuximab) | light chain = SEQ ID NO: 684 |
| anti-EGFR-TIM3ecd-LIGHTecd | heavy chain = SEQ ID NO: 230 |
| (cetuximab) | light chain = SEQ ID NO: 685 |
| anti-EGFR-TIM3ecd-OX40Lecd | heavy chain = SEQ ID NO: 230 |
| (cetuximab) | light chain = SEQ ID NO: 686 |
| anti-EGFR-VEGFRecd-41BBLecd | heavy chain = SEQ ID NO: 231 |
| (cetuximab) | light chain = SEQ ID NO: 677 |
| anti-EGFR-VEGFRecd-CD30Lecd | heavy chain = SEQ ID NO: 231 |
| (cetuximab) | light chain = SEQ ID NO: 678 |
| anti-EGFR-VEGFRecd-CD40Lecd | heavy chain = SEQ ID NO: 231 |
| (cetuximab) | light chain = SEQ ID NO: 679 |
| anti-EGFR-VEGFRecd-CD70ecd | heavy chain = SEQ ID NO: 231 |
| (cetuximab) | light chain = SEQ ID NO: 680 |
| anti-EGFR-VEGFRecd-GITRLecd | heavy chain = SEQ ID NO: 231 |
| (cetuximab) | light chain = SEQ ID NO: 681 |
| anti-EGFR-VEGFRecd-ICOSLecd | heavy chain = SEQ ID NO: 231 |
| (cetuximab) | light chain = SEQ ID NO: 682 |
| anti-EGFR-VEGFRecd-IL12ecd | heavy chain = SEQ ID NO: 231 |
| (cetuximab) | light chain = SEQ ID NO: 683 |
| anti-EGFR-VEGFRecd-IL15ecd | heavy chain = SEQ ID NO: 231 |
| (cetuximab) | light chain = SEQ ID NO: 684 |
| anti-EGFR-VEGFRecd-LIGHTecd | heavy chain = SEQ ID NO: 231 |
| (cetuximab) | light chain = SEQ ID NO: 685 |
| anti-EGFR-VEGFRecd-OX40Lecd | heavy chain = SEQ ID NO: 231 |
| (cetuximab) | light chain = SEQ ID NO: 686 |
| anti-IL17R-41BBLecd | heavy chain = SEQ ID NO: 687 |
| (brodalumab) | light chain = SEQ ID NO: 63 |
| anti-IL17R-41BBLecd-BTLAecd | heavy chain = SEQ ID NO: 687 |
| (brodalumab) | light chain = SEQ ID NO: 331 |
| anti-IL17R-41BBLecd-PD1ecd | heavy chain = SEQ ID NO: 687 |
| (brodalumab) | light chain = SEQ ID NO: 326 |
| anti-IL17R-41BBLecd-SIGLEC10ecd | heavy chain = SEQ ID NO: 687 |
| (brodalumab) | light chain = SEQ ID NO: 327 |
| anti-IL17R-41BBLecd-SIRPaecd | heavy chain = SEQ ID NO: 687 |
| (brodalumab) | light chain = SEQ ID NO: 328 |
| anti-IL17R-41BBLecd-TIM3ecd | heavy chain = SEQ ID NO: 687 |
| (brodalumab) | light chain = SEQ ID NO: 329 |
| anti-IL17R-CD30Lecd | heavy chain = SEQ ID NO: 688 |
| (brodalumab) | light chain = SEQ ID NO: 63 |
| anti-IL17R-CD30Lecd-BTLAecd | heavy chain = SEQ ID NO: 688 |
| (brodalumab) | light chain = SEQ ID NO: 331 |
| anti-IL17R-CD30Lecd-PD1ecd | heavy chain = SEQ ID NO: 688 |
| (brodalumab) | light chain = SEQ ID NO: 326 |
| anti-IL17R-CD30Lecd-SIGLEC10ecd | heavy chain = SEQ ID NO: 688 |
| (brodalumab) | light chain = SEQ ID NO: 327 |
| anti-IL17R-CD30Lecd-SIRPaecd | heavy chain = SEQ ID NO: 688 |
| (brodalumab) | light chain = SEQ ID NO: 328 |
| anti-IL17R-CD30Lecd-TIM3ecd | heavy chain = SEQ ID NO: 688 |
| (brodalumab) | light chain = SEQ ID NO: 329 |
| anti-IL17R-CD40Lecd | heavy chain = SEQ ID NO: 689 |
| (brodalumab) | light chain = SEQ ID NO: 63 |
| anti-IL17R-CD40Lecd-BTLAecd | heavy chain = SEQ ID NO: 689 |
| (brodalumab) | light chain = SEQ ID NO: 331 |
| anti-IL17R-CD40Lecd-PD1ecd | heavy chain = SEQ ID NO: 689 |
| (brodalumab) | light chain = SEQ ID NO: 326 |
| anti-IL17R-CD40Lecd-SIGLEC10ecd | heavy chain = SEQ ID NO: 689 |
| (brodalumab) | light chain = SEQ ID NO: 327 |
| anti-IL17R-CD40Lecd-SIRPaecd | heavy chain = SEQ ID NO: 689 |
| (brodalumab) | light chain = SEQ ID NO: 328 |
| anti-IL17R-CD40Lecd-TIM3ecd | heavy chain = SEQ ID NO: 689 |
| (brodalumab) | light chain = SEQ ID NO: 329 |
| anti-IL17R-CD70ecd | heavy chain = SEQ ID NO: 690 |
| (brodalumab) | light chain = SEQ ID NO: 63 |
| anti-IL17R-CD70ecd-BTLAecd | heavy chain = SEQ ID NO: 690 |
| (brodalumab) | light chain = SEQ ID NO: 331 |
| anti-IL17R-CD70ecd-PD1ecd | heavy chain = SEQ ID NO: 690 |
| (brodalumab | light chain = SEQ ID NO: 326 |
| anti-IL17R-CD70ecd-SIGLEC10ecd | heavy chain = SEQ ID NO: 690 |
| (brodalumab) | light chain = SEQ ID NO: 327 |
| anti-IL17R-CD70ecd-SIRPaecd | heavy chain = SEQ ID NO: 690 |
| (brodalumab) | light chain = SEQ ID NO: 328 |
| anti-IL17R-CD70ecd-TIM3ecd | heavy chain = SEQ ID NO: 690 |
| (brodalumab) | light chain = SEQ ID NO: 329 |
| anti-IL17R-GITRLecd | heavy chain = SEQ ID NO: 691 |
| (brodalumab) | light chain = SEQ ID NO: 63 |
| anti-IL17R-GITRLecd-BTLAecd | heavy chain = SEQ ID NO: 691 |
| (brodalumab) | light chain = SEQ ID NO: 331 |
| anti-IL17R-GITRLecd-PD1ecd | heavy chain = SEQ ID NO: 691 |
| (brodalumab) | light chain = SEQ ID NO: 326 |
| anti-IL17R-GITRLecd-SIGLEC10ecd | heavy chain = SEQ ID NO: 691 |
| (brodalumab) | light chain = SEQ ID NO: 327 |
| anti-IL17R-GITRLecd-SIRPaecd | heavy chain = SEQ ID NO: 691 |
| (brodalumab) | light chain = SEQ ID NO: 328 |
| anti-IL17R-GITRLecd-TIM3ecd | heavy chain = SEQ ID NO: 691 |
| (brodalumab) | light chain = SEQ ID NO: 329 |
| anti-IL17R-ICOSLecd | heavy chain = SEQ ID NO: 692 |
| (brodalumab) | light chain = SEQ ID NO: 63 |
| anti-IL17R-ICOSLecd-BTLAecd | heavy chain = SEQ ID NO: 692 |
| (brodalumab) | light chain = SEQ ID NO: 331 |
| anti-IL17R-ICOSLecd-PD1ecd | heavy chain = SEQ ID NO: 692 |
| (brodalumab) | light chain = SEQ ID NO: 326 |
| anti-IL17R-ICOSLecd-SIGLEC10ecd | heavy chain = SEQ ID NO: 692 |
| (brodalumab) | light chain = SEQ ID NO: 327 |
| anti-IL17R-ICOSLecd-SIRPaecd | heavy chain = SEQ ID NO: 692 |
| (brodalumab) | light chain = SEQ ID NO: 328 |
| anti-IL17R-ICOSLecd-TIM3ecd | heavy chain = SEQ ID NO: 692 |
| (brodalumab) | light chain = SEQ ID NO: 329 |
| anti-IL17R-IL12ecd | heavy chain = SEQ ID NO: 693 |
| (brodalumab) | light chain = SEQ ID NO: 63 |
| anti-IL17R-IL12ecd-BTLAecd | heavy chain = SEQ ID NO: 693 |
| (brodalumab) | light chain = SEQ ID NO: 331 |
| anti-IL17R-IL12ecd-PD1ecd | heavy chain = SEQ ID NO: 693 |
| (brodalumab) | light chain = SEQ ID NO: 326 |
| anti-IL17R-IL12ecd-SIGLEC10ecd | heavy chain = SEQ ID NO: 693 |
| (brodalumab) | light chain = SEQ ID NO: 327 |
| anti-IL17R-IL12ecd-SIRPaecd | heavy chain = SEQ ID NO: 693 |
| (brodalumab) | light chain = SEQ ID NO: 328 |
| anti-IL17R-IL12ecd-TIM3ecd | heavy chain = SEQ ID NO: 693 |
| (brodalumab) | light chain = SEQ ID NO: 329 |
| anti-IL17R-IL15ecd | heavy chain = SEQ ID NO: 694 |
| (brodalumab) | light chain = SEQ ID NO: 63 |
| anti-IL17R-IL15ecd-BTLAecd | heavy chain = SEQ ID NO: 694 |
| (brodalumab) | light chain = SEQ ID NO: 331 |
| anti-IL17R-IL15ecd-PD1ecd | heavy chain = SEQ ID NO: 694 |
| (brodalumab) | light chain = SEQ ID NO: 326 |
| anti-IL17R-IL15ecd-SIGLEC10ecd | heavy chain = SEQ ID NO: 694 |
| (brodalumab) | light chain = SEQ ID NO: 327 |
| anti-IL17R-IL15ecd-SIRPaecd | heavy chain = SEQ ID NO: 694 |
| (brodalumab) | light chain = SEQ ID NO: 328 |
| anti-IL17R-IL15ecd-TIM3ecd | heavy chain = SEQ ID NO: 694 |
| (brodalumab) | light chain = SEQ ID NO: 329 |
| anti-IL17R-LIGHTecd | heavy chain = SEQ ID NO: 695 |
| (brodalumab) | light chain = SEQ ID NO: 63 |
| anti-IL17R-LIGHTecd-BTLAecd | heavy chain = SEQ ID NO: 695 |
| (brodalumab) | light chain = SEQ ID NO: 331 |
| anti-IL17R-LIGHTecd-PD1ecd | heavy chain = SEQ ID NO: 695 |
| (brodalumab) | light chain = SEQ ID NO: 326 |
| anti-IL17R-LIGHTecd-SIGLEC10ecd | heavy chain = SEQ ID NO: 695 |
| (brodalumab) | light chain = SEQ ID NO: 327 |
| anti-IL17R-LIGHTecd-SIRPaecd | heavy chain = SEQ ID NO: 695 |
| (brodalumab) | light chain = SEQ ID NO: 328 |
| anti-IL17R-LIGHTecd-TIM3ecd | heavy chain = SEQ ID NO: 695 |
| (brodalumab) | light chain = SEQ ID NO: 329 |
| anti-IL17R-OX40Lecd | heavy chain = SEQ ID NO: 696 |
| (brodalumab) | light chain = SEQ ID NO: 63 |
| anti-IL17R-OX40Lecd-BTLAecd | heavy chain = SEQ ID NO: 696 |
| (brodalumab) | light chain = SEQ ID NO: 331 |
| anti-IL17R-OX40Lecd-PD1ecd | heavy chain = SEQ ID NO: 696 |
| (brodalumab) | light chain = SEQ ID NO: 326 |
| anti-IL17R-OX40Lecd-SIGLEC10ecd | heavy chain = SEQ ID NO: 696 |
| (brodalumab) | light chain = SEQ ID NO: 327 |
| anti-IL17R-OX40Lecd-SIRPaecd | heavy chain = SEQ ID NO: 696 |
| (brodalumab) | light chain = SEQ ID NO: 328 |
| anti-IL17R-OX40Lecd-TIM3ecd | heavy chain = SEQ ID NO: 696 |
| (brodalumab) | light chain = SEQ ID NO: 329 |
| anti-IL17R-BTLAecd-41BBLecd | heavy chain = SEQ ID NO: 325 |
| (brodalumab) | light chain = SEQ ID NO: 697 |
| anti-IL17R-BTLAecd-CD30Lecd | heavy chain = SEQ ID NO: 325 |
| (brodalumab) | light chain = SEQ ID NO: 698 |
| anti-IL17R-BTLAecd-CD40Lecd | heavy chain = SEQ ID NO: 325 |
| (brodalumab) | light chain = SEQ ID NO: 699 |
| anti-IL17R-BTLAecd-CD70ecd | heavy chain = SEQ ID NO: 325 |
| (brodalumab) | light chain = SEQ ID NO: 700 |
| anti-IL17R-BTLAecd-GITRLecd | heavy chain = SEQ ID NO: 325 |
| (brodalumab) | light chain = SEQ ID NO: 701 |
| anti-IL17R-BTLAecd-ICOSLecd | heavy chain = SEQ ID NO: 325 |
| (brodalumab) | light chain = SEQ ID NO: 702 |
| anti-IL17R-BTLAecd-IL12ecd | heavy chain = SEQ ID NO: 325 |
| (brodalumab) | light chain = SEQ ID NO: 703 |
| anti-IL17R-BTLAecd-IL15ecd | heavy chain = SEQ ID NO: 325 |
| (brodalumab) | light chain = SEQ ID NO: 704 |
| anti-IL17R-BTLAecd-LIGHTecd | heavy chain = SEQ ID NO: 325 |
| (brodalumab) | light chain = SEQ ID NO: 705 |
| anti-IL17R-BTLAecd-OX40Lecd | heavy chain = SEQ ID NO: 325 |
| (brodalumab) | light chain = SEQ ID NO: 706 |
| anti-IL17R-PD1ecd-41BBLecd | heavy chain = SEQ ID NO: 330 |
| (brodalumab) | light chain = SEQ ID NO: 697 |
| anti-IL17R-PD1ecd-CD30Lecd | heavy chain = SEQ ID NO: 330 |
| (brodalumab) | light chain = SEQ ID NO: 698 |
| anti-IL17R-PD1ecd-CD40Lecd | heavy chain = SEQ ID NO: 330 |
| (brodalumab) | light chain = SEQ ID NO: 699 |
| anti-IL17R-PD1ecd-CD70ecd | heavy chain = SEQ ID NO: 330 |
| (brodalumab) | light chain = SEQ ID NO: 700 |
| anti-IL17R-PD1ecd-GITRLecd | heavy chain = SEQ ID NO: 330 |
| (brodalumab) | light chain = SEQ ID NO: 701 |
| anti-IL17R-PD1ecd-ICOSLecd | heavy chain = SEQ ID NO: 330 |
| (brodalumab) | light chain = SEQ ID NO: 702 |
| anti-IL17R-PD1ecd-IL12ecd | heavy chain = SEQ ID NO: 330 |
| (brodalumab) | light chain = SEQ ID NO: 703 |
| anti-IL17R-PD1ecd-IL15ecd | heavy chain = SEQ ID NO: 330 |
| (brodalumab) | light chain = SEQ ID NO: 704 |
| anti-IL17R-PD1ecd-LIGHTecd | heavy chain = SEQ ID NO: 330 |
| (brodalumab) | light chain = SEQ ID NO: 705 |
| anti-IL17R-PD1ecd-OX40Lecd | heavy chain = SEQ ID NO: 330 |
| (brodalumab) | light chain = SEQ ID NO: 706 |
| anti-IL17R-SIGLEC10ecd-41BBLecd | heavy chain = SEQ ID NO: 332 |
| (brodalumab) | light chain = SEQ ID NO: 697 |
| anti-IL17R-SIGLEC10ecd-CD30Lecd | heavy chain = SEQ ID NO: 332 |
| (brodalumab) | light chain = SEQ ID NO: 698 |
| anti-IL17R-SIGLEC10ecd-CD40Lecd | heavy chain = SEQ ID NO: 332 |
| (brodalumab) | light chain = SEQ ID NO: 699 |
| anti-IL17R-SIGLEC10ecd-CD70ecd | heavy chain = SEQ ID NO: 332 |
| (brodalumab) | light chain = SEQ ID NO: 700 |
| anti-IL17R-SIGLEC10ecd-GITRLecd | heavy chain = SEQ ID NO: 332 |
| (brodalumab) | light chain = SEQ ID NO: 701 |
| anti-IL17R-SIGLEC10ecd-ICOSLecd | heavy chain = SEQ ID NO: 332 |
| (brodalumab) | light chain = SEQ ID NO: 702 |
| anti-IL17R-SIGLEC10ecd-IL12ecd | heavy chain = SEQ ID NO: 332 |
| (brodalumab) | light chain = SEQ ID NO: 703 |
| anti-IL17R-SIGLEC10ecd-IL15ecd | heavy chain = SEQ ID NO: 332 |
| (brodalumab) | light chain = SEQ ID NO: 704 |
| anti-IL17R-SIGLEC10ecd-LIGHTecd | heavy chain = SEQ ID NO: 332 |
| (brodalumab) | light chain = SEQ ID NO: 705 |
| anti-IL17R-SIGLEC10ecd-OX40Lecd | heavy chain = SEQ ID NO: 332 |
| (brodalumab) | light chain = SEQ ID NO: 706 |
| anti-IL17R-SIRPaecd-41BBLecd | heavy chain = SEQ ID NO: 333 |
| (brodalumab) | light chain = SEQ ID NO: 697 |
| anti-IL17R-SIRPaecd-CD30Lecd | heavy chain = SEQ ID NO: 333 |
| (brodalumab) | light chain = SEQ ID NO: 698 |
| anti-IL17R-SIRPaecd-CD40Lecd | heavy chain = SEQ ID NO: 333 |
| (brodalumab) | light chain = SEQ ID NO: 699 |
| anti-IL17R-SIRPaecd-CD70ecd | heavy chain = SEQ ID NO: 333 |
| (brodalumab) | light chain = SEQ ID NO: 700 |
| anti-IL17R-SIRPaecd-GITRLecd | heavy chain = SEQ ID NO: 333 |
| (brodalumab) | light chain = SEQ ID NO: 701 |
| anti-IL17R-SIRPaecd-ICOSLecd | heavy chain = SEQ ID NO: 333 |
| (brodalumab) | light chain = SEQ ID NO: 702 |
| anti-IL17R-SIRPaecd-IL12ecd | heavy chain = SEQ ID NO: 333 |
| (brodalumab) | light chain = SEQ ID NO: 703 |
| anti-IL17R-SIRPaecd-IL15ecd | heavy chain = SEQ ID NO: 333 |
| (brodalumab) | light chain = SEQ ID NO: 704 |
| anti-IL17R-SIRPaecd-LIGHTecd | heavy chain = SEQ ID NO: 333 |
| (brodalumab) | light chain = SEQ ID NO: 705 |
| anti-IL17R-SIRPaecd-OX40Lecd | heavy chain = SEQ ID NO: 333 |
| (brodalumab) | light chain = SEQ ID NO: 706 |
| anti-IL17R-TGFbRecd-41BBLecd | heavy chain = SEQ ID NO: 334 |
| (brodalumab) | light chain = SEQ ID NO: 697 |
| anti-IL17R-TGFbRecd-CD30Lecd | heavy chain = SEQ ID NO: 334 |
| (brodalumab) | light chain = SEQ ID NO: 698 |
| anti-IL17R-TGFbRecd-CD40Lecd | heavy chain = SEQ ID NO: 334 |
| (brodalumab) | light chain = SEQ ID NO: 699 |
| anti-IL17R-TGFbRecd-CD70ecd | heavy chain = SEQ ID NO: 334 |
| (brodalumab) | light chain = SEQ ID NO: 700 |
| anti-IL17R-TGFbRecd-GITRLecd | heavy chain = SEQ ID NO: 334 |
| (brodalumab) | light chain = SEQ ID NO: 701 |
| anti-IL17R-TGFbRecd-ICOSLecd | heavy chain = SEQ ID NO: 334 |
| (brodalumab) | light chain = SEQ ID NO: 702 |
| anti-IL17R-TGFbRecd-IL12ecd | heavy chain = SEQ ID NO: 334 |
| (brodalumab) | light chain = SEQ ID NO: 703 |
| anti-IL17R-TGFbRecd-IL15ecd | heavy chain = SEQ ID NO: 334 |
| (brodalumab) | light chain = SEQ ID NO: 704 |
| anti-IL17R-TGFbRecd-LIGHTecd | heavy chain = SEQ ID NO: 334 |
| (brodalumab) | light chain = SEQ ID NO: 705 |
| anti-IL17R-TGFbRecd-OX40Lecd | heavy chain = SEQ ID NO: 334 |
| (brodalumab) | light chain = SEQ ID NO: 706 |
| anti-IL17R-TIM3ecd-41BBLecd | heavy chain = SEQ ID NO: 335 |
| (brodalumab) | light chain = SEQ ID NO: 697 |
| anti-IL17R-TIM3ecd-CD30Lecd | heavy chain = SEQ ID NO: 335 |
| (brodalumab) | light chain = SEQ ID NO: 698 |
| anti-IL17R-TIM3ecd-CD40Lecd | heavy chain = SEQ ID NO: 335 |
| (brodalumab) | light chain = SEQ ID NO: 699 |
| anti-IL17R-TIM3ecd-CD70ecd | heavy chain = SEQ ID NO: 335 |
| (brodalumab) | light chain = SEQ ID NO: 700 |
| anti-IL17R-TIM3ecd-GITRLecd | heavy chain = SEQ ID NO: 335 |
| (brodalumab) | light chain = SEQ ID NO: 701 |
| anti-IL17R-TIM3ecd-ICOSLecd | heavy chain = SEQ ID NO: 335 |
| (brodalumab) | light chain = SEQ ID NO: 702 |
| anti-IL17R-TIM3ecd-IL12ecd | heavy chain = SEQ ID NO: 335 |
| (brodalumab) | light chain = SEQ ID NO: 703 |
| anti-IL17R-TIM3ecd-IL15ecd | heavy chain = SEQ ID NO: 335 |
| (brodalumab) | light chain = SEQ ID NO: 704 |
| anti-IL17R-TIM3ecd-LIGHTecd | heavy chain = SEQ ID NO: 335 |
| (brodalumab) | light chain = SEQ ID NO: 705 |
| anti-IL17R-TIM3ecd-OX40Lecd | heavy chain = SEQ ID NO: 335 |
| (brodalumab) | light chain = SEQ ID NO: 706 |
| anti-IL17R-VEGFRecd-41BBLecd | heavy chain = SEQ ID NO: 336 |
| (brodalumab) | light chain = SEQ ID NO: 697 |
| anti-IL17R-VEGFRecd-CD30Lecd | heavy chain = SEQ ID NO: 336 |
| (brodalumab) | light chain = SEQ ID NO: 698 |
| anti-IL17R-VEGFRecd-CD40Lecd | heavy chain = SEQ ID NO: 336 |
| (brodalumab) | light chain = SEQ ID NO: 699 |
| anti-IL17R-VEGFRecd-CD70ecd | heavy chain = SEQ ID NO: 336 |
| (brodalumab) | light chain = SEQ ID NO: 700 |
| anti-IL17R-VEGFRecd-GITRLecd | heavy chain = SEQ ID NO: 336 |
| (brodalumab) | light chain = SEQ ID NO: 701 |
| anti-IL17R-VEGFRecd-ICOSLecd | heavy chain = SEQ ID NO: 336 |
| (brodalumab) | light chain = SEQ ID NO: 702 |
| anti-IL17R-VEGFRecd-IL12ecd | heavy chain = SEQ ID NO: 336 |
| (brodalumab) | light chain = SEQ ID NO: 703 |
| anti-IL17R-VEGFRecd-IL15ecd | heavy chain = SEQ ID NO: 336 |
| (brodalumab) | light chain = SEQ ID NO: 704 |
| anti-IL17R-VEGFRecd-LIGHTecd | heavy chain = SEQ ID NO: 336 |
| (brodalumab) | light chain = SEQ ID NO: 705 |
| anti-IL17R-VEGFRecd-OX40Lecd | heavy chain = SEQ ID NO: 336 |
| (brodalumab) | light chain = SEQ ID NO: 706 |
| anti-VEGF-41BBLecd | heavy chain = SEQ ID NO: 707 |
| (bevacizumab) | light chain = SEQ ID NO: 32 |
| anti-VEGF-41BBLecd-BTLAecd | heavy chain = SEQ ID NO: 707 |
| (bevacizumab) | light chain = SEQ ID NO: 367 |
| anti-VEGF-41BBLecd-PD1ecd | heavy chain = SEQ ID NO: 707 |
| (bevacizumab) | light chain = SEQ ID NO: 362 |
| anti-VEGF-41BBLecd-SIGLEC10ecd | heavy chain = SEQ ID NO: 707 |
| (bevacizumab) | light chain = SEQ ID NO: 363 |
| anti-VEGF-41BBLecd-SIRPaecd | heavy chain = SEQ ID NO: 707 |
| (bevacizumab) | light chain = SEQ ID NO: 364 |
| anti-VEGF-41BBLecd-TIM3ecd | heavy chain = SEQ ID NO: 707 |
| (bevacizumab) | light chain = SEQ ID NO: 365 |
| anti-VEGF-CD30Lecd | heavy chain = SEQ ID NO: 708 |
| (bevacizumab) | light chain = SEQ ID NO: 32 |
| anti-VEGF-CD30Lecd-BTLAecd | heavy chain = SEQ ID NO: 708 |
| (bevacizumab) | light chain = SEQ ID NO: 367 |
| anti-VEGF-CD30Lecd-PD1ecd | heavy chain = SEQ ID NO: 708 |
| (bevacizumab) | light chain = SEQ ID NO: 362 |
| anti-VEGF-CD30Lecd-SIGLEC10ecd | heavy chain = SEQ ID NO: 708 |
| (bevacizumab) | light chain = SEQ ID NO: 363 |
| anti-VEGF-CD30Lecd-SIRPaecd | heavy chain = SEQ ID NO: 708 |
| (bevacizumab) | light chain = SEQ ID NO: 364 |
| anti-VEGF-CD30Lecd-TIM3ecd | heavy chain = SEQ ID NO: 708 |
| (bevacizumab) | light chain = SEQ ID NO: 365 |
| anti-VEGF-CD40Lecd | heavy chain = SEQ ID NO: 709 |
| (bevacizumab) | light chain = SEQ ID NO: 32 |
| anti-VEGF-CD40Lecd-BTLAecd | heavy chain = SEQ ID NO: 709 |
| (bevacizumab) | light chain = SEQ ID NO: 367 |
| anti-VEGF-CD40Lecd-PD1ecd | heavy chain = SEQ ID NO: 709 |
| (bevacizumab) | light chain = SEQ ID NO: 362 |
| anti-VEGF-CD40Lecd-SIGLEC10ecd | heavy chain = SEQ ID NO: 709 |
| (bevacizumab) | light chain = SEQ ID NO: 363 |
| anti-VEGF-CD40Lecd-SIRPaecd | heavy chain = SEQ ID NO: 709 |
| (bevacizumab) | light chain = SEQ ID NO: 364 |
| anti-VEGF-CD40Lecd-TIM3ecd | heavy chain = SEQ ID NO: 709 |
| (bevacizumab) | light chain = SEQ ID NO: 365 |
| anti-VEGF-CD70ecd | heavy chain = SEQ ID NO: 710 |
| (bevacizumab) | light chain = SEQ ID NO: 32 |
| anti-VEGF-CD70ecd-BTLAecd | heavy chain = SEQ ID NO: 710 |
| (bevacizumab) | light chain = SEQ ID NO: 367 |
| anti-VEGF-CD70ecd-PD1ecd | heavy chain = SEQ ID NO: 710 |
| (bevacizumab) | light chain = SEQ ID NO: 362 |
| anti-VEGF-CD70ecd-SIGLEC10ecd | heavy chain = SEQ ID NO: 710 |
| (bevacizumab) | light chain = SEQ ID NO: 363 |
| anti-VEGF-CD70ecd-SIRPaecd | heavy chain = SEQ ID NO: 710 |
| (bevacizumab) | light chain = SEQ ID NO: 364 |
| anti-VEGF-CD70ecd-TIM3ecd | heavy chain = SEQ ID NO: 710 |
| (bevacizumab) | light chain = SEQ ID NO: 365 |
| anti-VEGF-GITRLecd | heavy chain = SEQ ID NO: 711 |
| (bevacizumab) | light chain = SEQ ID NO: 32 |
| anti-VEGF-GITRLecd-BTLAecd | heavy chain = SEQ ID NO: 711 |
| (bevacizumab) | light chain = SEQ ID NO: 367 |
| anti-VEGF-GITRLecd-PD1ecd | heavy chain = SEQ ID NO: 711 |
| (bevacizumab) | light chain = SEQ ID NO: 362 |
| anti-VEGF-GITRLecd-SIGLEC10ecd | heavy chain = SEQ ID NO: 711 |
| (bevacizumab) | light chain = SEQ ID NO: 363 |
| anti-VEGF-GITRLecd-SIRPaecd | heavy chain = SEQ ID NO: 711 |
| (bevacizumab) | light chain = SEQ ID NO: 364 |
| anti-VEGF-GITRLecd-TIM3ecd | heavy chain = SEQ ID NO: 711 |
| (bevacizumab) | light chain = SEQ ID NO: 365 |
| anti-VEGF-ICOSLecd | heavy chain = SEQ ID NO: 712 |
| (bevacizumab) | light chain = SEQ ID NO: 32 |
| anti-VEGF-ICOSLecd-BTLAecd | heavy chain = SEQ ID NO: 712 |
| (bevacizumab) | light chain = SEQ ID NO: 367 |
| anti-VEGF-ICOSLecd-PD1ecd | heavy chain = SEQ ID NO: 712 |
| (bevacizumab) | light chain = SEQ ID NO: 362 |
| anti-VEGF-ICOSLecd-SIGLEC10ecd | heavy chain = SEQ ID NO: 712 |
| (bevacizumab) | light chain = SEQ ID NO: 363 |
| anti-VEGF-ICOSLecd-SIRPaecd | heavy chain = SEQ ID NO: 712 |
| (bevacizumab) | light chain = SEQ ID NO: 364 |
| anti-VEGF-ICOSLecd-TIM3ecd | heavy chain = SEQ ID NO: 712 |
| (bevacizumab) | light chain = SEQ ID NO: 365 |
| anti-VEGF-IL12ecd | heavy chain = SEQ ID NO: 713 |
| (bevacizumab) | light chain = SEQ ID NO: 32 |
| anti-VEGF-IL12ecd-BTLAecd | heavy chain = SEQ ID NO: 713 |
| (bevacizumab) | light chain = SEQ ID NO: 367 |
| anti-VEGF-IL12ecd-PD1ecd | heavy chain = SEQ ID NO: 713 |
| (bevacizumab) | light chain = SEQ ID NO: 362 |
| anti-VEGF-IL12ecd-SIGLEC10ecd | heavy chain = SEQ ID NO: 713 |
| (bevacizumab) | light chain = SEQ ID NO: 363 |
| anti-VEGF-IL12ecd-SIRPaecd | heavy chain = SEQ ID NO: 713 |
| (bevacizumab) | light chain = SEQ ID NO: 364 |
| anti-VEGF-IL12ecd-TIM3ecd | heavy chain = SEQ ID NO: 713 |
| (bevacizumab) | light chain = SEQ ID NO: 365 |
| anti-VEGF-IL15ecd | heavy chain = SEQ ID NO: 714 |
| (bevacizumab) | light chain = SEQ ID NO: 32 |
| anti-VEGF-IL15ecd-BTLAecd | heavy chain = SEQ ID NO: 714 |
| (bevacizumab) | light chain = SEQ ID NO: 367 |
| anti-VEGF-IL15ecd-PD1ecd | heavy chain = SEQ ID NO: 714 |
| (bevacizumab) | light chain = SEQ ID NO: 362 |
| anti-VEGF-IL15ecd-SIGLEC10ecd | heavy chain = SEQ ID NO: 714 |
| (bevacizumab) | light chain = SEQ ID NO: 363 |
| anti-VEGF-IL15ecd-SIRPaecd | heavy chain = SEQ ID NO: 714 |
| (bevacizumab) | light chain = SEQ ID NO: 364 |
| anti-VEGF-IL15ecd-TIM3ecd | heavy chain = SEQ ID NO: 714 |
| (bevacizumab) | light chain = SEQ ID NO: 365 |
| anti-VEGF-LIGHTecd | heavy chain = SEQ ID NO: 715 |
| (bevacizumab) | light chain = SEQ ID NO: 32 |
| anti-VEGF-LIGHTecd-BTLAecd | heavy chain = SEQ ID NO: 715 |
| (bevacizumab) | light chain = SEQ ID NO: 367 |
| anti-VEGF-LIGHTecd-PD1ecd | heavy chain = SEQ ID NO: 715 |
| (bevacizumab) | light chain = SEQ ID NO: 362 |
| anti-VEGF-LIGHTecd-SIGLEC10ecd | heavy chain = SEQ ID NO: 715 |
| (bevacizumab) | light chain = SEQ ID NO: 363 |
| anti-VEGF-LIGHTecd-SIRPaecd | heavy chain = SEQ ID NO: 715 |
| (bevacizumab) | light chain = SEQ ID NO: 364 |
| anti-VEGF-LIGHTecd-TIM3ecd | heavy chain = SEQ ID NO: 715 |
| (bevacizumab) | light chain = SEQ ID NO: 365 |
| anti-VEGF-OX40Lecd | heavy chain = SEQ ID NO: 716 |
| (bevacizumab) | light chain = SEQ ID NO: 32 |
| anti-VEGF-OX40Lecd-BTLAecd | heavy chain = SEQ ID NO: 716 |
| (bevacizumab) | light chain = SEQ ID NO: 367 |
| anti-VEGF-OX40Lecd-PD1ecd | heavy chain = SEQ ID NO: 716 |
| (bevacizumab) | light chain = SEQ ID NO: 362 |
| anti-VEGF-OX40Lecd-SIGLEC10ecd | heavy chain = SEQ ID NO: 716 |
| (bevacizumab) | light chain = SEQ ID NO: 363 |
| anti-VEGF-OX40Lecd-SIRPaecd | heavy chain = SEQ ID NO: 716 |
| (bevacizumab) | light chain = SEQ ID NO: 364 |
| anti-VEGF-OX40Lecd-TIM3ecd | heavy chain = SEQ ID NO: 716 |
| (bevacizumab) | light chain = SEQ ID NO: 365 |
| anti-VEGF-BTLAecd-41BBLecd | heavy chain = SEQ ID NO: 361 |
| (bevacizumab) | light chain = SEQ ID NO: 717 |
| anti-VEGF-BTLAecd-CD30Lecd | heavy chain = SEQ ID NO: 361 |
| (bevacizumab) | light chain = SEQ ID NO: 718 |
| anti-VEGF-BTLAecd-CD40Lecd | heavy chain = SEQ ID NO: 361 |
| (bevacizumab) | light chain = SEQ ID NO: 719 |
| anti-VEGF-BTLAecd-CD70ecd | heavy chain = SEQ ID NO: 361 |
| (bevacizumab) | light chain = SEQ ID NO: 720 |
| anti-VEGF-BTLAecd-GITRLecd | heavy chain = SEQ ID NO: 361 |
| (bevacizumab) | light chain = SEQ ID NO: 721 |
| anti-VEGF-BTLAecd-ICOSLecd | heavy chain = SEQ ID NO: 361 |
| (bevacizumab) | light chain = SEQ ID NO: 722 |
| anti-VEGF-BTLAecd-IL12ecd | heavy chain = SEQ ID NO: 361 |
| (bevacizumab) | light chain = SEQ ID NO: 723 |
| anti-VEGF-BTLAecd-IL15ecd | heavy chain = SEQ ID NO: 361 |
| (bevacizumab) | light chain = SEQ ID NO: 724 |
| anti-VEGF-BTLAecd-LIGHTecd | heavy chain = SEQ ID NO: 361 |
| (bevacizumab) | light chain = SEQ ID NO: 725 |
| anti-VEGF-BTLAecd-OX40Lecd | heavy chain = SEQ ID NO: 361 |
| (bevacizumab) | light chain = SEQ ID NO: 726 |
| anti-VEGF-PD1ecd-41BBLecd | heavy chain = SEQ ID NO: 366 |
| (bevacizumab) | light chain = SEQ ID NO: 717 |
| anti-VEGF-PD1ecd-CD30Lecd | heavy chain = SEQ ID NO: 366 |
| (bevacizumab) | light chain = SEQ ID NO: 718 |
| anti-VEGF-PD1ecd-CD40Lecd | heavy chain = SEQ ID NO: 366 |
| (bevacizumab) | light chain = SEQ ID NO: 719 |
| anti-VEGF-PD1ecd-CD70ecd | heavy chain = SEQ ID NO: 366 |
| (bevacizumab) | light chain = SEQ ID NO: 720 |
| anti-VEGF-PD1ecd-GITRLecd | heavy chain = SEQ ID NO: 366 |
| (bevacizumab) | light chain = SEQ ID NO: 721 |
| anti-VEGF-PD1ecd-ICOSLecd | heavy chain = SEQ ID NO: 366 |
| (bevacizumab) | light chain = SEQ ID NO: 722 |
| anti-VEGF-PD1ecd-IL12ecd | heavy chain = SEQ ID NO: 366 |
| (bevacizumab) | light chain = SEQ ID NO: 723 |
| anti-VEGF-PD1ecd-IL15ecd | heavy chain = SEQ ID NO: 366 |
| (bevacizumab) | light chain = SEQ ID NO: 724 |
| anti-VEGF-PD1ecd-LIGHTecd | heavy chain = SEQ ID NO: 366 |
| (bevacizumab) | light chain = SEQ ID NO: 725 |
| anti-VEGF-PD1ecd-OX40Lecd | heavy chain = SEQ ID NO: 366 |
| (bevacizumab) | light chain = SEQ ID NO: 726 |
| anti-VEGF-SIGLEC10ecd-41BBLecd | heavy chain = SEQ ID NO: 368 |
| (bevacizumab) | light chain = SEQ ID NO: 717 |
| anti-VEGF-SIGLEC10ecd-CD30Lecd | heavy chain = SEQ ID NO: 368 |
| (bevacizumab) | light chain = SEQ ID NO: 718 |
| anti-VEGF-SIGLEC10ecd-CD40Lecd | heavy chain = SEQ ID NO: 368 |
| (bevacizumab) | light chain = SEQ ID NO: 719 |
| anti-VEGF-SIGLEC10ecd-CD70ecd | heavy chain = SEQ ID NO: 368 |
| (bevacizumab) | light chain = SEQ ID NO: 720 |
| anti-VEGF-SIGLEC10ecd-GITRLecd | heavy chain = SEQ ID NO: 368 |
| (bevacizumab) | light chain = SEQ ID NO: 721 |
| anti-VEGF-SIGLEC10ecd-ICOSLecd | heavy chain = SEQ ID NO: 368 |
| (bevacizumab) | light chain = SEQ ID NO: 722 |
| anti-VEGF-SIGLEC10ecd-IL12ecd | heavy chain = SEQ ID NO: 368 |
| (bevacizumab) | light chain = SEQ ID NO: 723 |
| anti-VEGF-SIGLEC10ecd-IL15ecd | heavy chain = SEQ ID NO: 368 |
| (bevacizumab) | light chain = SEQ ID NO: 724 |
| anti-VEGF-SIGLEC10ecd-LIGHTecd | heavy chain = SEQ ID NO: 368 |
| (bevacizumab) | light chain = SEQ ID NO: 725 |
| anti-VEGF-SIGLEC10ecd-OX40Lecd | heavy chain = SEQ ID NO: 368 |
| (bevacizumab) | light chain = SEQ ID NO: 726 |
| anti-VEGF-SIRPaecd-41BBLecd | heavy chain = SEQ ID NO: 369 |
| (bevacizumab) | light chain = SEQ ID NO: 717 |
| anti-VEGF-SIRPaecd-CD30Lecd | heavy chain = SEQ ID NO: 369 |
| (bevacizumab) | light chain = SEQ ID NO: 718 |
| anti-VEGF-SIRPaecd-CD40Lecd | heavy chain = SEQ ID NO: 369 |
| (bevacizumab) | light chain = SEQ ID NO: 719 |
| anti-VEGF-SIRPaecd-CD70ecd | heavy chain = SEQ ID NO: 369 |
| (bevacizumab) | light chain = SEQ ID NO: 720 |
| anti-VEGF-SIRPaecd-GITRLecd | heavy chain = SEQ ID NO: 369 |
| (bevacizumab) | light chain = SEQ ID NO: 721 |
| anti-VEGF-SIRPaecd-ICOSLecd | heavy chain = SEQ ID NO: 369 |
| (bevacizumab) | light chain = SEQ ID NO: 722 |
| anti-VEGF-SIRPaecd-IL12ecd | heavy chain = SEQ ID NO: 369 |
| (bevacizumab) | light chain = SEQ ID NO: 723 |
| anti-VEGF-SIRPaecd-IL15ecd | heavy chain = SEQ ID NO: 369 |
| (bevacizumab) | light chain = SEQ ID NO: 724 |
| anti-VEGF-SIRPaecd-LIGHTecd | heavy chain = SEQ ID NO: 369 |
| (bevacizumab) | light chain = SEQ ID NO: 725 |
| anti-VEGF-SIRPaecd-OX40Lecd | heavy chain = SEQ ID NO: 369 |
| (bevacizumab) | light chain = SEQ ID NO: 726 |
| anti-VEGF-TGFbRecd-41BBLecd | heavy chain = SEQ ID NO: 370 |
| (bevacizumab) | light chain = SEQ ID NO: 717 |
| anti-VEGF-TGFbRecd-CD30Lecd | heavy chain = SEQ ID NO: 370 |
| (bevacizumab) | light chain = SEQ ID NO: 718 |
| anti-VEGF-TGFbRecd-CD40Lecd | heavy chain = SEQ ID NO: 370 |
| (bevacizumab) | light chain = SEQ ID NO: 719 |
| anti-VEGF-TGFbRecd-CD70ecd | heavy chain = SEQ ID NO: 370 |
| (bevacizumab) | light chain = SEQ ID NO: 720 |
| anti-VEGF-TGFbRecd-GITRLecd | heavy chain = SEQ ID NO: 370 |
| (bevacizumab) | light chain = SEQ ID NO: 721 |
| anti-VEGF-TGFbRecd-ICOSLecd | heavy chain = SEQ ID NO: 370 |
| (bevacizumab) | light chain = SEQ ID NO: 722 |
| anti-VEGF-TGFbRecd-IL12ecd | heavy chain = SEQ ID NO: 370 |
| (bevacizumab) | light chain = SEQ ID NO: 723 |
| anti-VEGF-TGFbRecd-IL15ecd | heavy chain = SEQ ID NO: 370 |
| (bevacizumab) | light chain = SEQ ID NO: 724 |
| anti-VEGF-TGFbRecd-LIGHTecd | heavy chain = SEQ ID NO: 370 |
| (bevacizumab) | light chain = SEQ ID NO: 725 |
| anti-VEGF-TGFbRecd-OX40Lecd | heavy chain = SEQ ID NO: 370 |
| (bevacizumab) | light chain = SEQ ID NO: 726 |
| anti-VEGF-TIM3ecd-41BBLecd | heavy chain = SEQ ID NO: 371 |
| (bevacizumab) | light chain = SEQ ID NO: 717 |
| anti-VEGF-TIM3ecd-CD30Lecd | heavy chain = SEQ ID NO: 371 |
| (bevacizumab) | light chain = SEQ ID NO: 718 |
| anti-VEGF-TIM3ecd-CD40Lecd | heavy chain = SEQ ID NO: 371 |
| (bevacizumab) | light chain = SEQ ID NO: 719 |
| anti-VEGF-TIM3ecd-CD70ecd | heavy chain = SEQ ID NO: 371 |
| (bevacizumab) | light chain = SEQ ID NO: 720 |
| anti-VEGF-TIM3ecd-GITRLecd | heavy chain = SEQ ID NO: 371 |
| (bevacizumab) | light chain = SEQ ID NO: 721 |
| anti-VEGF-TIM3ecd-ICOSLecd | heavy chain = SEQ ID NO: 371 |
| (bevacizumab) | light chain = SEQ ID NO: 722 |
| anti-VEGF-TIM3ecd-IL12ecd | heavy chain = SEQ ID NO: 371 |
| (bevacizumab) | light chain = SEQ ID NO: 723 |
| anti-VEGF-TIM3ecd-IL15ecd | heavy chain = SEQ ID NO: 371 |
| (bevacizumab) | light chain = SEQ ID NO: 724 |
| anti-VEGF-TIM3ecd-LIGHTecd | heavy chain = SEQ ID NO: 371 |
| (bevacizumab) | light chain = SEQ ID NO: 725 |
| anti-VEGF-TIM3ecd-OX40Lecd | heavy chain = SEQ ID NO: 371 |
| (bevacizumab) | light chain = SEQ ID NO: 726 |
| anti-CEACAM5/CD3 (cibisatamab) fused | heavy chains = SEQ ID NOs: 747, 748 |
| to BTLA on HC | light chains = SEQ ID NOs: 218, 219 |
| anti-CEACAM5/CD3 (cibisatamab) fused | heavy chains = SEQ ID NOs: 216, 217 |
| to BTLA on LC | light chains = SEQ ID NOs: 749, 219 |
| anti-CEACAM5/CD3 (cibisatamab) fused | heavy chains = SEQ ID NOs: 216, 217 |
| to BTLA on LC | light chains = SEQ ID NOs: 218, 750 |
| anti-CEACAM5/CD3 (cibisatamab) fused | heavy chains = SEQ ID NOs: 751, 752 |
| to PD1 onHC | light chains = SEQ ID NOs: 218, 219 |
| anti-CEACAM5/CD3 (cibisatamab) fused | heavy chains = SEQ ID NOs: 216, 217 |
| to PD1 on LC | light chains = SEQ ID NOs: 753, 219 |
| anti-CEACAM5/CD3 (cibisatamab) fused | heavy chains = SEQ ID NOs: 216, 217 |
| to PD1 on LC | light chains = SEQ ID NOs: 218, 754 |
| anti-CEACAM5/CD3 (cibisatamab) fused | heavy chains = SEQ ID NOs: 755, 756 |
| to SIGLEC10 on HC | light chains = SEQ ID NOs: 218, 219 |
| anti-CEACAM5/CD3 (cibisatamab) fused | heavy chains = SEQ ID Nos: 216, 217 |
| to SIGLEC10 on LC | light chains = SEQ ID Nos: 757, 219 |
| anti-CEACAM5/CD3 (cibisatamab) fused | heavy chains = SEQ ID NOs: 216, 217 |
| to SIGLEC10 on LC | light chains = SEQ ID NOs: 218, 758 |
| anti-CEACAM5/CD3 (cibisatamab) fused | heavy chains = SEQ ID NOs: 759, 760 |
| to SIRPa on HC | light chains = SEQ ID NOs: 218, 219 |
| anti-CEACAM5/CD3 (cibisatamab) fused | heavy chains = SEQ ID NOs: 216, 217 |
| to SIRPa on LC | light chains = SEQ ID NOs: 761, 219 |
| anti-CEACAM5/CD3 (cibisatamab) fused | heavy chains = SEQ ID NOs: 216, 217 |
| to SIRPa on LC | light chains = SEQ ID NOs: 218, 762 |
| anti-CEACAM5/CD3 (cibisatamab) fused | heavy chains = SEQ ID NOs: 763, 764 |
| to TGFbR on HC | light chains = SEQ ID NOs: 218, 219 |
| anti-CEACAM5/CD3 (cibisatamab) fused | heavy chains = SEQ ID NOs: 765, 766 |
| to TIM3 on HC | light chains = SEQ ID NOs: 218, 219 |
| anti-CEACAM5/CD3 (cibisatamab) fused | heavy chains = SEQ ID NOs: 216, 217 |
| to TIM3 on LC | light chains = SEQ ID NOs: 767, 219 |
| anti-CEACAM5/CD3 (cibisatamab) fused | heavy chains = SEQ ID NOs: 216, 217 |
| to TIM3 on LC | light chains = SEQ ID NOs: 218, 768 |
| anti-CEACAM5/CD3 (cibisatamab) fused | heavy chains = SEQ ID NOs: 769, 770 |
| to VEGFR on HC | light chains = SEQ ID NOs: 218, 219 |
| anti-CD3/CD19 (blinatumomab) fused | SEQ ID NO: 727 |
| to BTLA | |
| anti-CD3/CD19 (blinatumomab) fused | SEQ ID NO: 728 |
| to BTLA | |
| anti-CD3/PSMA (pasotuxizumab) fused | SEQ ID NO: 729 |
| to BTLA | |
| anti-CD3/PSMA (pasotuxizumab) fused | SEQ ID NO: 730 |
| to BTLA | |
| anti-CD3/CD19 (blinatumomab) fused | SEQ ID NO: 731 |
| to PD1 | |
| anti-CD3/CD19 (blinatumomab) fused | SEQ ID NO: 732 |
| to PD1 | |
| anti-CD3/PSMA (pasotuxizumab) fused | SEQ ID NO: 733 |
| to PD1 | |
| anti-CD3/PSMA (pasotuxizumab) fused | SEQ ID NO: 734 |
| to PD1 | |
| anti-CD3/CD19 (blinatumomab) fused | SEQ ID NO: 735 |
| to SIGLEC10 | |
| anti-CD3/CD19 (blinatumomab) fused | SEQ ID NO: 736 |
| to SIGLEC10 | |
| anti-CD3/PSMA (pasotuxizumab) fused | SEQ ID NO: 737 |
| to SIGLEC10 | |
| anti-CD3/PSMA (pasotuxizumab) fused | SEQ ID NO: 738 |
| to SIGLEC10 | |
| anti-CD3/CD19 (blinatumomab) fused | SEQ ID NO: 739 |
| to SIRPa | |
| anti-CD3/CD19 (blinatumomab) fused | SEQ ID NO: 740 |
| to SIRPa | |
| anti-CD3/PSMA (pasotuxizumab) fused | SEQ ID NO: 741 |
| to SIRPa | |
| anti-CD3/PSMA (pasotuxizumab) fused | SEQ ID NO: 742 |
| to SIRPa | |
| anti-CD3/CD19 (blinatumomab) fused | SEQ ID NO: 743 |
| to TIM3 | |
| anti-CD3/CD19 (blinatumomab) fused | SEQ ID NO: 744 |
| to TIM3 | |
| anti-CD3/PSMA (pasotuxizumab) fused | SEQ ID NO: 745 |
| to TIM3 | |
| anti-CD3/PSMA (pasotuxizumab) fused | SEQ ID NO: 746 |
| to TIM3 | |

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Methods

The amino acid sequences of exemplary fusion proteins of the invention were codon optimized with GeneOptimizer®. The cDNA for the antibody heavy chain and the cDNA for the antibody light chain were gene synthesized and subsequently cloned into separate plasmids (pEvi3; evitria AG, Switzerland) under the control of a mammalian promoter and polyadenylation signal. Plasmid DNA was amplified in E. coli and DNA was purified using anion exchange kits for low endotoxin plasmid DNA preparation. DNA concentration was determined by measuring the absorption at a wavelength of 260 nm. Correctness of the sequences was verified with Sanger sequencing (with up to two sequencing reactions per plasmid depending on the size of the cDNA.) The plasmid DNAs for heavy and light chain were subsequently co-transfected into suspension-adapted CHO K1 cells (originally received from ATCC and adapted to serum-free growth in suspension culture at evitria). The seed was grown in eviGrow medium, a chemically defined, animal-component free, serum-free medium. Cells were transfected with eviFect (evitria AG, Switzerland). and the CHO cells were cultured in eviMake2 (evitria AG, Switzerland), a serum-free, animal-component free medium. Production was terminated once viability reached 75%, which occurred at day 8 after transfection. Supernatant was harvested by centrifugation and subsequent filtration (0.2 um filter). The antibody was purified using MabSelect™ Sure™ (Protein A affinity chromatography on a Bio-Rad BioLogic FuoFlow FPLC machine with subsequent gel filtration as polishing and rebuffering step). In some cases, the antibody was further purified using SEC purification.

The fusion proteins of the invention can also be produced via stable transfection of a mammalian cell line (e.g. CHO K1 cells) with plasmid DNA encoding the chains of the fusion protein, selection of stably transfected cell clones or cell pools expressing the fusion protein, development of a Master Cell Bank for production of the fusion protein, purification of the fusion protein by Protein A affinity chromatography and/or SEC, and formulation using methods well described in the art.

### Example 2

### Anti-PDL1-BTLAecd, Anti-PDL1-TGFbRII, Anti-PDL1-TGFbRIIecd-BTLAecd, Anti-PD1-BTLAecd, Anti-PD1-TGFbRIIecd, and Anti-PD1-TGFbRIIecd-BTLAecd Fusion Proteins

Anti-PDL1-BTLAecd was designed to target both PD-L1 and BTLA ligand (HVEM) by fusing the C-terminus of the heavy chain of an anti-PDL1 antibody with a ligand binding sequence of the extracellular domain of human BTLA (BTLAecd) via a flexible linker peptide, (GGGGS)3. Anti-PDL1-TGFbRIIecd-BTLAecd was designed to target PD-L1, TGFb, and BLTA ligands (HVEM) by fusing the C-terminus of the heavy chain of an anti-PDL1 antibody with a ligand binding sequence of the extracellular domain of human TGFbRII (TGFbRIIecd) via a flexible linker peptide, (GGGGS)3, and fusing the C-terminus of the light chain of an anti-PDL1 antibody with a ligand binding sequence of the extracellular domain of human BTLA (BTLAecd) via a flexible linker peptide, (GGGGS)3.

Anti-PD1-BTLAecd was designed to target both PD-1 and BTLA ligand (HVEM) by fusing the C-terminus of the heavy chain of an anti-PD1 antibody (pembrolizumab) with a ligand binding sequence of the extracellular domain of human BTLA (BTLAecd) via a flexible linker peptide, (GGGGS)3. Anti-PD1-TGFbRIIecd was designed to target PD-1 and TGFb by fusing the C-terminus of the heavy chain of an anti-PD1 antibody (pembrolizumab) with a ligand binding sequence of the extracellular domain of human TGFbRII (TGFbRIIecd) via a flexible linker peptide, (GGGGS)3. Anti-PD1-TGFbRIIecd-BTLAecd was designed to target PD-1, TGFb, and BTLA ligands (HVEM) by fusing the C-terminus of the heavy chain of an anti-PD1 antibody (pembrolizumab) with a ligand binding sequence of the extracellular domain of human TGFbRII (TGFbRIIecd) via a flexible linker peptide, (GGGGS)3, and fusing the C-terminus of the light chain of an anti-PD1 antibody with a ligand binding sequence of the extracellular domain of human BTLA (BTLAecd) via a flexible linker peptide, (GGGGS)3.

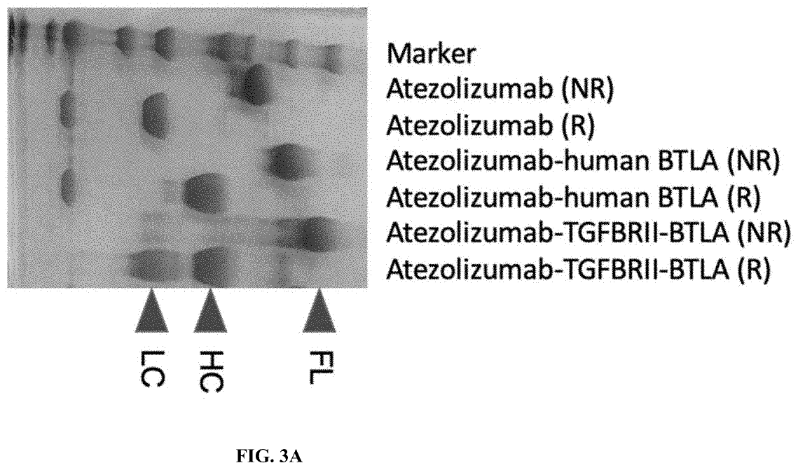

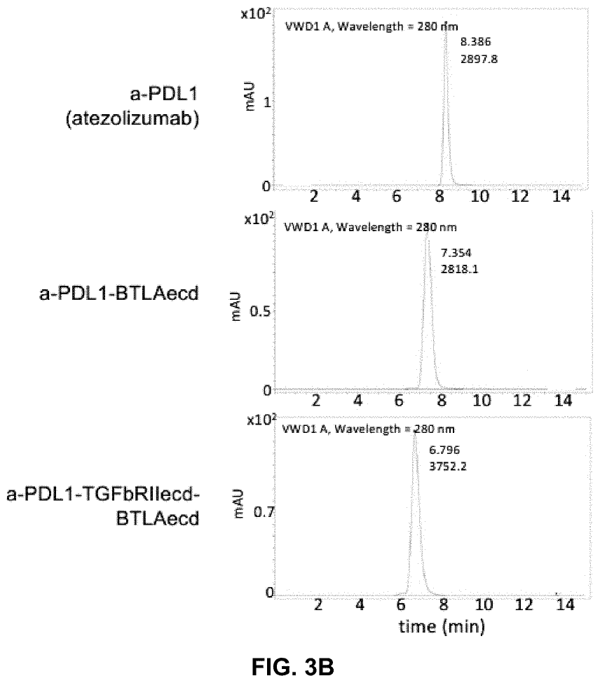

FIG. 3 shows the characterization of anti-PDL1-BTLAecd, anti-PDL1-TGFbRII and anti-PDL1-TGFbRIIecd-BTLAecd. SDS-PAGE under reducing (R) and non-reducing (NR) conditions was used to compare the full-length (FL), heavy chain (HC) and light chain (LC) of anti-PDL1 (atezolizumab), anti-PDL1-BTLAecd and anti-PDL1-TGFbRIIecd-BTLAecd (FIG. 3A). SDS-PAGE confirmed the expected higher molecular weight of the heavy chain of anti-PDL1-BTLAecd compared to the heavy chain of anti-PDL1 antibody. SDS-PAGE of anti-PDL1-BTLAecd confirmed the expected higher molecular weight of the heavy chain compared to the heavy chain of anti-PDL1 antibody (Light chain of anti-PDL1-BTLAecd is identical to anti-PDL1). SDS-PAGE of anti-PDL1-TGFbRIIecd-BTLAecd confirmed the expected higher molecular weight of the heavy chain (anti-PDL1 HC fused to TGFbRIIecd) compared to the heavy chain of anti-PDL1 antibody, and the expected higher molecular weight of the light chain (anti-PDL1 LC fused to BTLAecd) compared to the light chain of anti-PDL1.

SEC-HPLC analysis of anti-PDL1 (atezolizumab), anti-PDL1-BTLAecd and anti-PDL1-TGFbRIIecd-BTLAecd (>99% monomericity) (FIG. 3B).

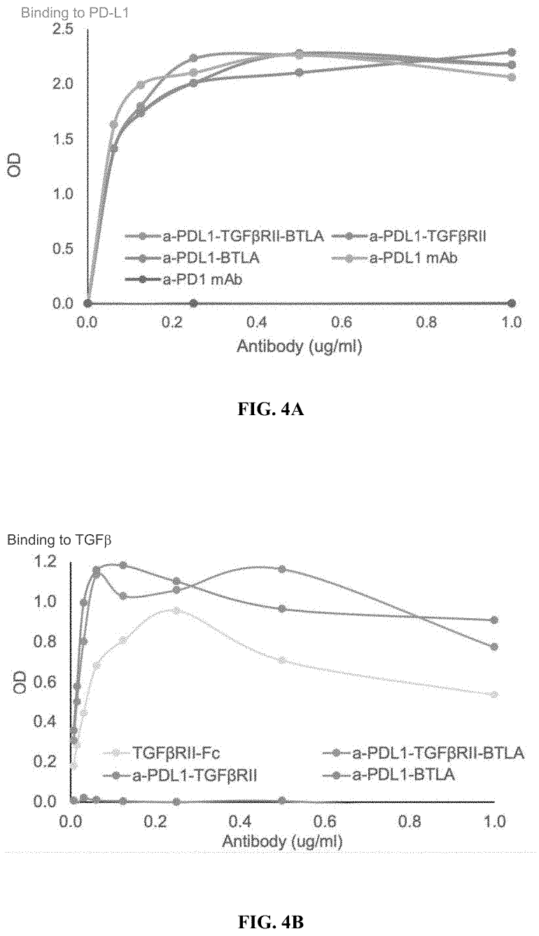

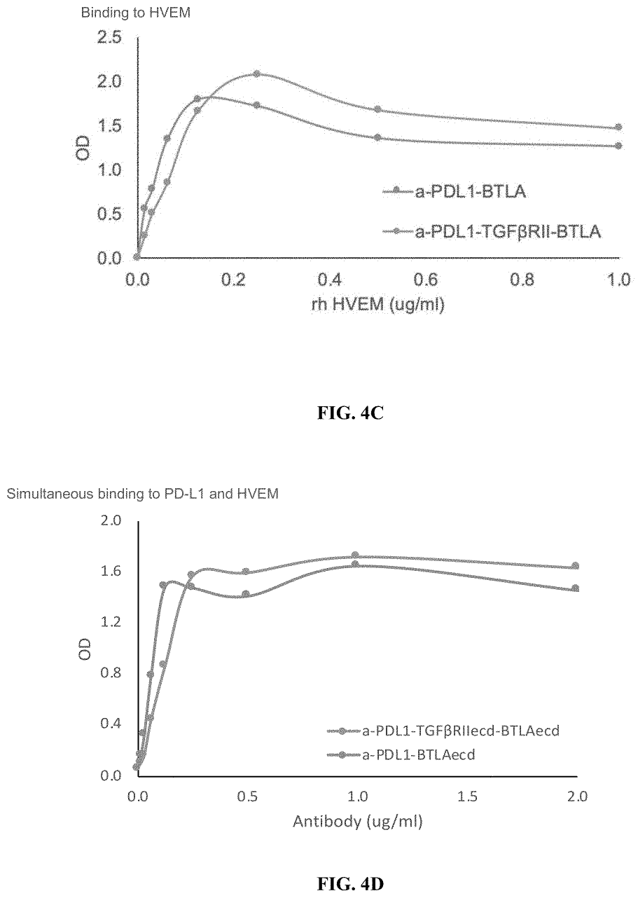

FIG. 4 shows the target binding ability of anti-PDL1-BTLAecd, anti-PDL1-TGFbRII and anti-PDL1-TGFbRIIecd-BTLAecd. Standard ELISA showing the ability of anti-PDL1 (atezolizumab), anti-PDL1-BTLAecd and anti-PDL1-TGFbRIIecd-BTLAecd to bind PD-L1. Plate was coated with the indicated amount of each antibody or ALT (0-1 ug/mL) followed by addition of biotinylated huPD-L1 (1 ug/ml) (FIG. 4A). Binding to biotinylated PD-L1 was detected by avidin-HRP (ELISA). Plate coated with anti-PD1 (nivolumab) served as a negative control (FIG. 4A).

Standard ELISA showing the ability of anti-PDL1-TGFbRIIecd and anti-PDL1-TGFbRIIecd-BTLAecd to bind TGFb. Plate was coated with the indicated amount of anti-PDL1-TGFbRIIecd, anti-PDL1-TGFbRIIecd-BTLAecd, anti-PDL1-BTLAecd, or TGFbRIIecd-Fc (0-1 ug/mL) followed by addition of biotinylated TGFb (1 ng/ml) (FIG. 4B). Binding to biotinylated TGFb was detected by avidin-HRP (ELISA). Plate coated with TGFbRIIecd-Fc served as a positive control, and plate coated with anti-PDL1-BTLAecd served as a negative control (FIG. 4B).

Standard ELISA showing the ability of anti-PDL1-BTLAecd and anti-PDL1-TGFbRIIecd-BTLAecd to bind the BTLA ligand HVEM. Plate was coated with 5 ug/mL of each antibody-ligand trap (anti-PDL1-BTLAecd and anti-PDL1-TGFbRIIecd-BTLAecd) followed by addition of varying amounts of biotinylated huHVEM (0-1 ug/ml). Binding to biotinylated HuHVEM was detected by avidin-HRP (ELISA) (FIG. 4C).

ELISA showing the ability of anti-PDL1-BTLAecd and anti-PDL1-TGFbRIIecd-BTLAecd to simultaneously bind PD-L1 and the BTLA ligand HVEM (FIG. 4D). Varying amounts of each antibody (0-2 ug/mL; anti-PDL1-BTLAecd or anti-PDL1-TGFbRIIecd-BTLAecd) were added to plate coated with 1 ug/mL PDL1-Fc, and then washed before addition of 500 ng/mL biotinylated huHVEM. The ability of PD-L1-bound anti-PDL1-BTLAecd or anti-PDL1-TGFbRIIecd-BTLAecd to simultaneously bind biotinylated huHVEM (via BTLAecd fused to the heavy or light chain respectively) was detected by avidin-HRP (ELISA).

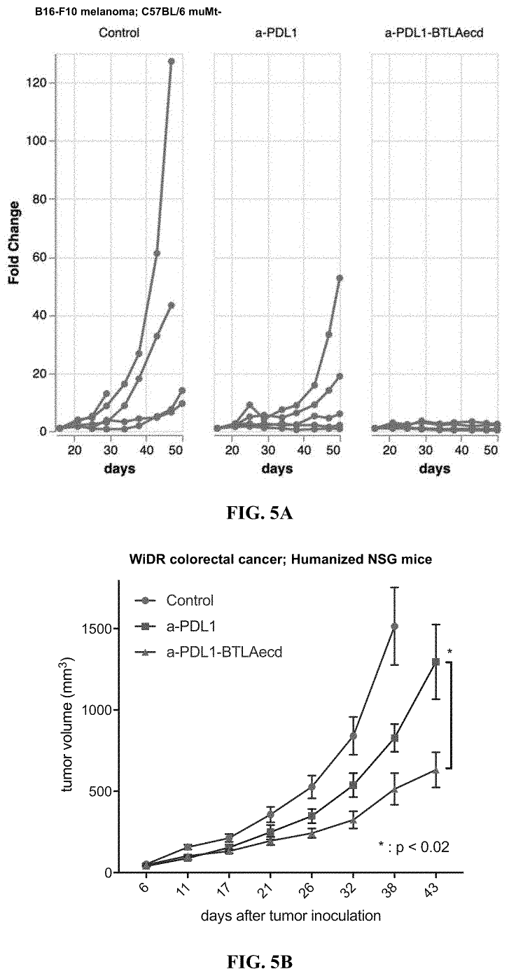

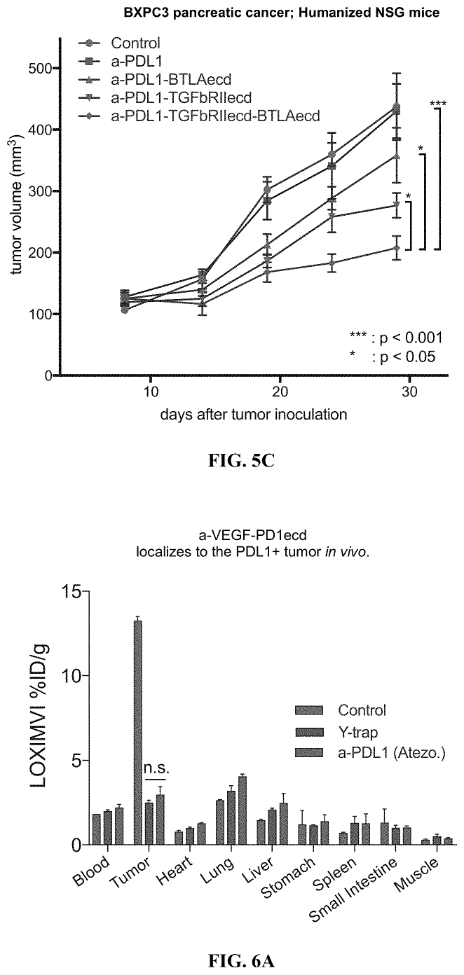

The ability of anti-PDL1, anti-PDL1-BTLAecd to elicit antitumor immunity and inhibit the growth of syngeneic B16-F10 tumors in C57BL/6 muMt-mice is shown in FIG. 5A. B16-F10 murine melanoma tumor cells were subcutaneously inoculated in B cell-deficient C57BL/6 muMt- (B cell deficient) mice (1×105 tumor cells/mouse). Mice bearing tumor xenografts were randomized into groups (n=5 per group) and treated with vehicle alone (control) or each of the following antibodies (5 mg/kg i.p/week): anti-PDL1 (atezolizumab) or anti-PDL1-BTLAecd. Treatment with anti-PDL1-BTLAecd was significantly more effective in inhibiting tumor growth compared to anti-PD-L1 (atezolizumab).

The ability of anti-PDL1, anti-PDL1-BTLAecd to elicit antitumor immunity and inhibit the growth of cancers was examined in human immune reconstituted NSG mice bearing human WiDR-colorectal cancer cells is shown in FIG. 5B. NSG mice immune were reconstituted with tumor-matched HLA A2+ human CD34+ HSC followed by subcutaneous implantation of WiDR tumor xenografts (3×106 tumor cells in Matrigel). Humanized mice bearing tumor xenografts were randomized into groups (n=6 per group) and treated with vehicle alone (control) or each of the following antibodies (5 mg/kg i.p q 6d): anti-PDL1 (atezolizumab), anti-PDL1-BTLAecd. Treatment with anti-PDL1-BTLAecd was significantly more effective in inhibiting tumor growth compared to anti-PD-L1 (atezolizumab)(p<0.02).

The ability of anti-PDL1, anti-PDL1-BTLAecd, anti-PDL1-TGFbRIIecd and anti-PDL1-BTLAecd-TGFbRIIecd to elicit antitumor immunity and inhibit the growth of cancers was examined in human immune reconstituted NSG mice bearing human BXPC3-pancreatic cancer cells. NSG mice immune were reconstituted with tumor-matched HLA A2+ human CD34+ HSC followed by subcutaneous implantation of BXPC3 tumor xenografts (3×106 tumor cells in Matrigel) is shown in FIG. 5C. Humanized mice bearing tumor xenografts were randomized into groups (n=6 per group) and treated with vehicle alone (control) or each of the following antibodies (5 mg/kg i.p q 6d): anti-PDL1 (atezolizumab), anti-PDL1-BTLAecd, anti-PDL1-TGFbRIIecd and anti-PDL1-BTLAecd-TGFbRIIecd. Treatment with anti-PDL1-BTLAecd-TGFbRIIecd was significantly more effective in inhibiting tumor growth compared to anti-PD-L1 (atezolizumab)(p<0.001), anti-PDL1-BTLAecd, or anti-PDL1-TGFbRIIecd (p<0.05).

These data demonstrate that BTLA ECD fused to the heavy or light chain of an antibody is capable of binding HVEM, thereby disrupting native BTLA-mediated SHP1/SHP2 inhibition and promoting HVEM-mediated co-stimulatory signaling. Furthermore, these data demonstrate that inhibition of BTLA/HVEM signaling with a decoy BTLA receptor ECD fused to a polypeptide that binds and disables another immuno-inhibitory molecule is effective in the treatment of cancer. Furthermore, these data demonstrate that the decoy BTLA receptor ECD fused to a polypeptide that binds and disables another T cell co-inhibitory molecule is effective in the treatment of cancer. Furthermore, these data demonstrate that decoy BTLA receptor ECD fused to a polypeptide that inhibits the interaction of a cytokine and its cytokine receptor is effective in the treatment of cancer. Furthermore, these data demonstrate that decoy BTLA receptor ECD fused to a polypeptide that binds a tumor cell surface molecule is effective in the treatment of cancer. Furthermore, these data demonstrate that decoy BTLA receptor ECD on either the heavy or light chain of the targeting polypeptide is capable of binding HVEM, thereby disrupting native BTLA-mediated SHP1/SHP2 inhibition and promoting HVEM-mediated co-stimulatory signaling, even while another receptor ECD is additionally fused to the antibody. Furthermore, these data demonstrate that decoy BTLA receptor ECD is effective in the treatment of cancer when part of a fusion protein comprising an additional ECD of a cytokine or cytokine receptor. Furthermore, these data demonstrate that decoy BTLA receptor ECD fused to an antibody can enable recruitment of T cells to tumor cells, since these data show that BTLA ECD can bind HVEM while the targeting antibody simultaneously binds a T cell surface molecule.

### Example 3

### Anti-VEGF-PD1 Fusion Protein

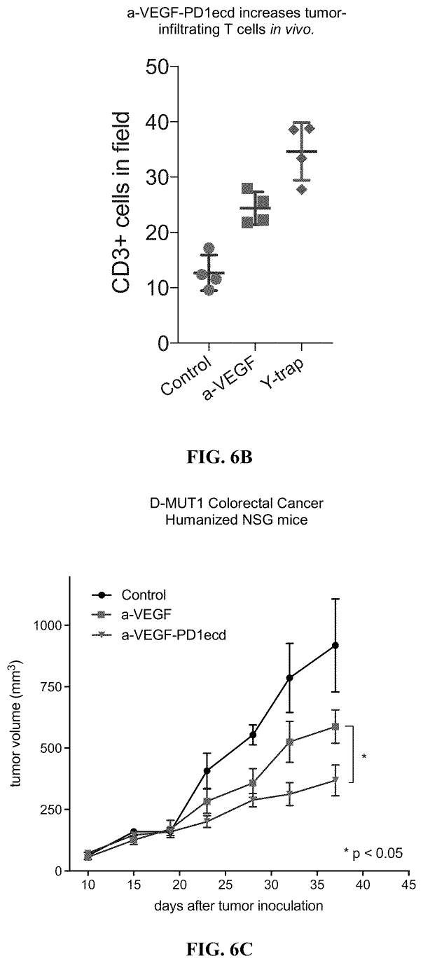

Mice are treated with mAbs 24 h prior to the radiotracer injection. Tissues are collected at 60 min after radiotracer injection, weighed and counted for radioactivity. Data is normalized for tissue weight and injected dose and presented as % ID/g. The radiotracer comprises a labeled high-affinity PDL1-binding peptide. Low % ID/g indicates effective competition with the labeled PD-L1 binding peptide. anti-VEGF-PD1 competes as effectively as anti-PDL1 mAb for binding PD-L1 in the tumor (FIG. 6A). CD3+ are counted in immunohistochemistry images of tumors in control group. anti-VEGF group, or anti-VEGF-PD1 ECD group. Treatment with anti-VEGF-PD1 results in significant increase of CD3+ cells (FIG. 6B). NSG mice immune reconstituted with tumor-matched HLA A2+ human CD34+ HSC and bearing KRAS mutant D-MUT1 human colorectal cancer tumor xenografts were treated (5 mg/kg i.p. weekly) with vehicle alone (untreated control) or the following antibodies (either alone or in combination), as indicated: with either vehicle alone (untreated control) or the following antibodies: anti-VEGF-PD1ecd; anti-VEGF (bevacizumab) [>=5 mice/group] (FIG. 6C). Tumor size was measured blinded to the treatment group and tumor volume was calculated using the formula (length×width×height). In vivo tumor growth curves (mean+SEM) are shown. p values were derived using unpaired, two-sided t-test.

These data demonstrate that a fusion protein that blocks VEGF and also comprises the ECD of a molecule that inhibits immune cells (e.g., T cells, dendritic cells, macrophages) is effective in treating cancer. These data demonstrate that a fusion protein that blocks VEGF and also comprises the ECD of a T cell co-inhibitory molecule is effective in treating cancer. These data demonstrate that a fusion protein that blocks VEGF and also comprises a polypeptide that binds a tumor cell surface molecule or molecule enriched in the tumor microenvironment is effective in localizing VEGF to the tumor microenvironment.

### Example 4

### Anti-VEGF-TGFbRII-PD1 Fusion Protein

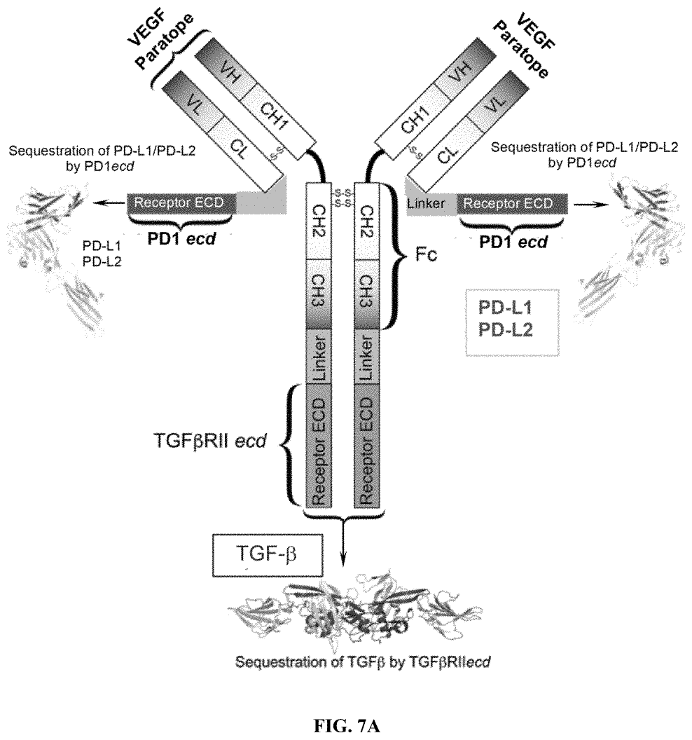

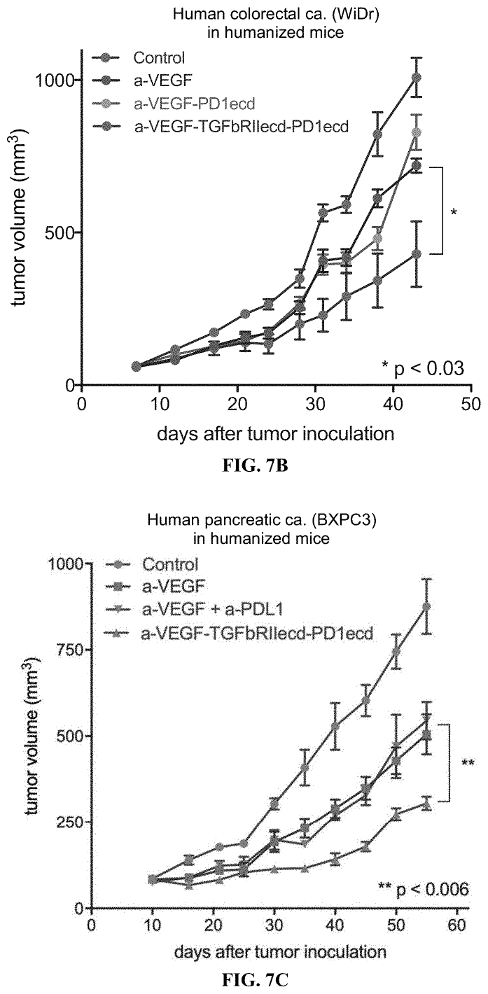

Structure of anti-VEGF-TGFbRII-PD1 (FIG. 7A). anti-VEGF binds VEGF, TGFbRII binds TGFb, PD1 binds PD-L1 and PD-L2. NSG mice immune were reconstituted with tumor-matched HLA A2+ human CD34+ HSC followed by subcutaneous implantation of WiDR tumor xenografts (1.5×106 tumor cells in Matrigel) (FIG. 7B). Humanized mice bearing tumor xenografts were randomized into groups (n=5 per group) and treated with vehicle alone (control) or equimolar doses of each of the following antibodies (i.p q6d): anti-VEGF, anti-VEGF-PD1, anti-VEGF-TGFbRII-PD1. Treatment with anti-VEGF-TGFbRII-PD1 was significantly more effective in inhibiting tumor growth compared to anti-VEGF-PD1 or anti-VEGF. NSG mice immune were reconstituted with tumor-matched HLA A2+ human CD34+ HSC followed by subcutaneous implantation of BXPC3 tumor xenografts (2×106 tumor cells in Matrigel) (FIG. 7C). Humanized mice bearing tumor xenografts were randomized into groups (n=5 per group) and treated with vehicle alone (control) or equimolar doses of each of the following antibodies (i.p q6d): anti-VEGF, anti-VEGF+anti-PDL1, anti-VEGF-TGFbRII-PD1. Treatment with anti-VEGF-TGFbRII-PD1 was significantly more effective in inhibiting tumor growth compared to anti-VEGF+anti-PDL1 or anti-VEGF.

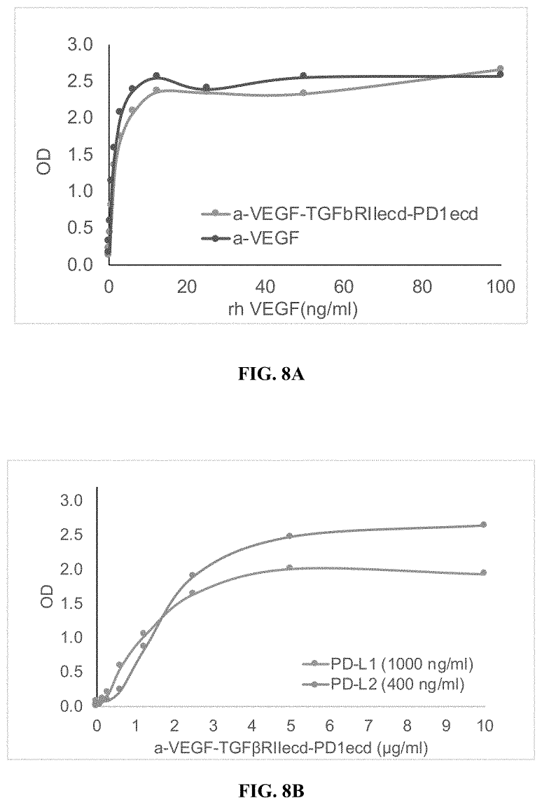

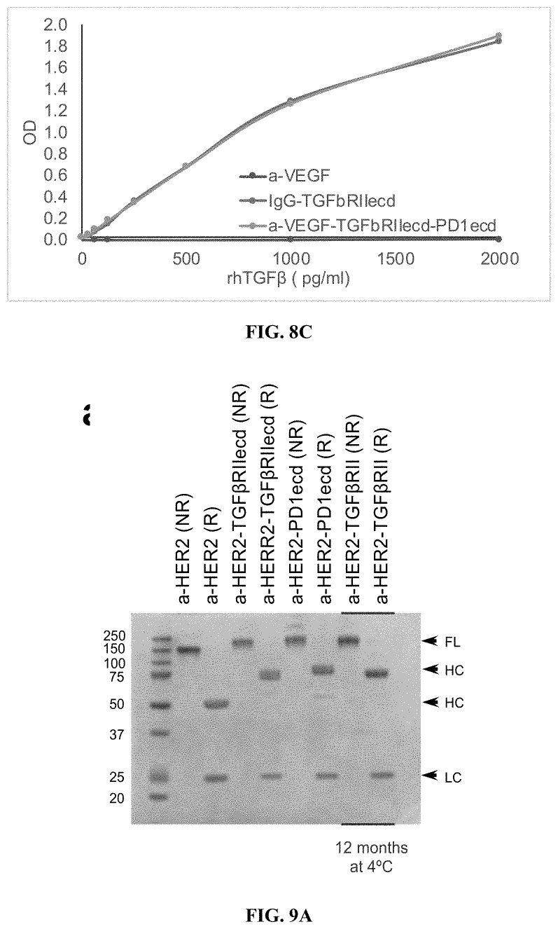

Standard ELISA showing the ability of anti-VEGF (bevacizumab), anti-VEGF-TGFbRII-PD1 to bind VEGF (FIG. 8A). Plate was coated with 2 ug/mL of anti-VEGF-PD1-TGFbRII or anti-VEGF (bevacizumab), followed by addition of biotinylated huVEGF in the amount indicated. Binding to biotinylated VEGF was detected by avidin-HRP (ELISA).

Standard ELISA showing the ability of anti-VEGF-TGFbRII-PD1 to bind TGFb (FIG. 8B). Plate was coated with the indicated amount of anti-VEGF-TGFbRII-PD1 (0-10 ug/mL) followed by addition of biotinylated PD-L1 (1000 ng/ml) or biotinylated PD-L1 (400 ng/mL). Binding to biotinylated PD-L1/PD-L2 was detected by avidin-HRP (ELISA).

Standard ELISA showing the ability of anti-VEGF-TGFbRII-PD1 to bind TGFb as well as IgG-TGFbRII (FIG. 8C). Plate was coated with 1 ug/mL of anti-VEGF-TGFbRII-PD1, IgG-TGFbRII as positive control, or anti-VEGF (bevacizumab) as negative control, followed by addition of varying amounts of biotinylated huTGFb1 (0-2000 pg/ml). Binding to biotinylated huTGFb1 was detected by avidin-HRP (ELISA)

These data demonstrate that blockade of TGFb and VEGF is more effective in treatment of cancer than blockade of VEGF alone. These data further demonstrate that a fusion protein comprising an anti-VEGF/VEGFR polypeptide and another polypeptide that inhibits angiogenesis (e.g., TGFb) is effective in the treatment of cancer. These data further demonstrate that a fusion protein comprising an antibody that inhibits angiogenesis fused to a receptor ECD that inhibits angiogenesis is effective in the treatment of cancer.

These data further demonstrate that a fusion protein comprising anti-VEGF/VEGFR antibody and a receptor ECD that inhibits angiogenesis is effective in the treatment of cancer. These data further demonstrate that a fusion protein comprising an anti-angiogenic polypeptide and a polypeptide that inhibits a key determinant of TH17 differentiation is effective in the treatment of cancer. These data further demonstrate that localized blockade of VEGF and/or TGFb in the tumor microenvironment is effective in the treatment of cancer.

### Example 5

### Anti-HER2-TGFbRII, Anti-HER2-PD1, Anti-EGFR-TGFbRII Fusion Proteins

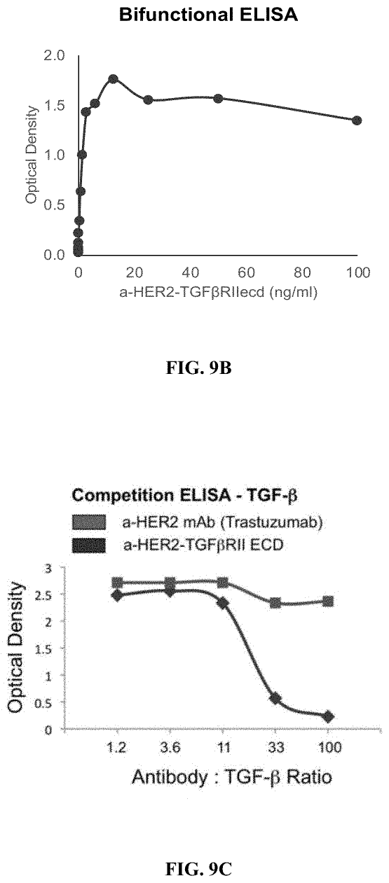

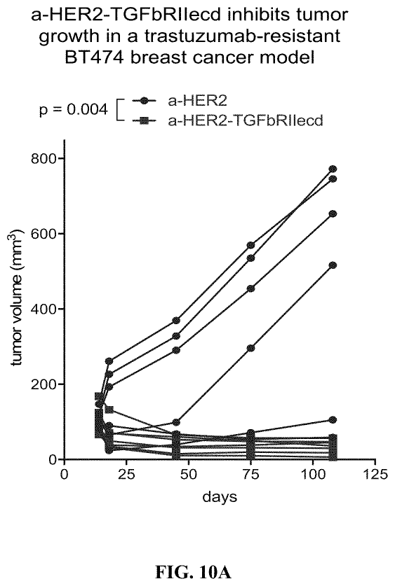

SDS-PAGE under reducing (R) and non-reducing (NR) conditions was used to compare the full-length (FL), heavy chain (HC) and light chain (LC) of anti-HER2-TGFβRIIecd, anti-HER2-PD1ecd, and anti-HER2 (Trastuzumab) (FIG. 9A). The assay was repeated after storage of anti-HER2-TGFbRIIecd for 12 months at 4° C. to ensure its stability (right-most two lanes; marked in red).

The ability of anti-HER2-TGFβRII to simultaneously bind HER2 and TGF-β1 was evaluated by a ‘double-sandwich’ ELISA wherein anti-HER2-TGFβRIIecd was added to HER2-Fc coated plates, followed by rhTGF-β1 (1 ng/ml) that was detected by a biotinylated anti-huTGF-β1 antibody (FIG. 9B). The plate was incubated with Avidin-HRP and developed with TMB substrate. anti-HER2-TGFβRIIecd exhibited simultaneous binding to HER2 and TGF-β1.

The ability of anti-HER2-TGFβRII to bind TGF-β1 was also evaluated by competition immunoassays (FIG. 9C). The ELISA plate was coated with capture antibody (anti-TGF-β Ab, 1 μg/ml), followed by rhTGF-β1 in the presence of either anti-HER2-TGFβRII or anti-HER2 mAb (TGF-β1: antibody ratio, 1:1 to 1:100) for 1 h at RT. Unlike anti-HER2 mAb, anti-HER2-TGFβRII antibody exhibited the ability to compete for binding to TGF-β1.

The HER2-overexpressing human breast cancer cell line BT-474 was cultured in vitro in the presence of escalating concentrations of anti-HER2 mAb (trastuzumab: 5 μg/ml-20 μg/ml) for 3 months. Trastuzumab-resistant cells that continued to grow in the presence of trastuzumab (20 μg/ml) for 30 days were isolated and implanted subcutaneously into the R flank of 4-6 week female BALB/c nude mice bearing estrogen pellets (8×10⁶ cells/mouse). At 21d following tumor cell inoculation, the mice were randomized and treated with anti-HER2 mAb (Trastuzumab) (5 mg/kg, i.p, weekly×6 wks). anti-HER2 mAb failed to stop tumor progression.

To evaluate the antitumor activity of anti-HER2-TGFβRIIecd against tumors that resisted anti-HER2 mAb in vivo (TrastuzumabR BT-474), trastuzumab-resistant tumors harvested from trastuzumab-treated F1 mice were sectioned into 2×2 mm pieces and implanted subcutaneously into a second cohort of female BALB/c nude mice (TrastuzumabR BT-474-F2). Tumor-bearing F2 mice were treated with either trastuzumab (5 mg/kg, i.p, every 2 weeks×6) or anti-HER2-TGFβRIIecd (5 mg/kg i.p. every 2 weeks×6).

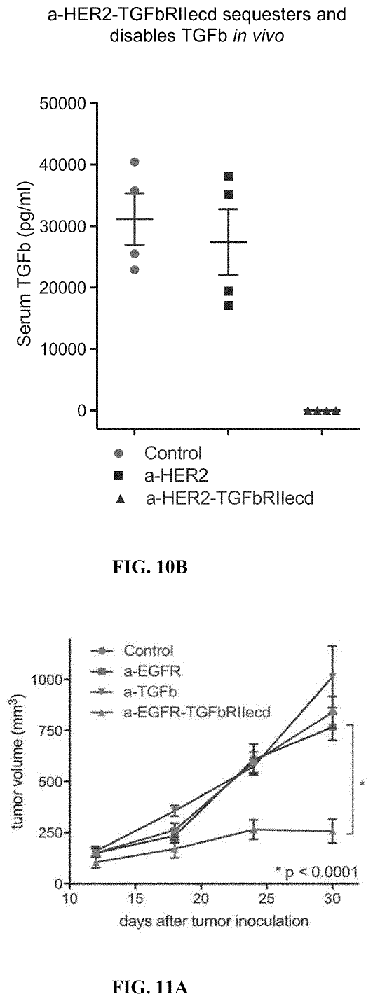

Residual tumors in TrastuzumabR BT-474 (BT-474-TR) F2 tumor-bearing mice following treatment with anti-HER2-TGFβRII were significantly smaller (mean±SEM=31.7±6.5) than those in F2 mice treated with anti-HER2 mAb (mean±SEM=453.9±121.4)(p=0.003) (FIG. 10A).

Whereas treatment of F2 mice bearing TrastuzumabR BT-474 tumor xenografts with trastuzumab showed continued tumor progression in 4/6 mice, all 7 mice treated with anti-HER2-TGFβRII showed complete inhibition of tumor growth (p=0.009).

Serum was collected from TrastuzumabR BT-474 tumor-bearing mice. A hydrochloric acid pre-treatment was performed, and serum concentrations of TGF-β1 were measured by ELISA (FIG. 10B).

In vivo treatment of tumor-bearing mice with anti-HER2-TGFbRIIecd completely sequestered activated serum TGFβ1 7 days after treatment.

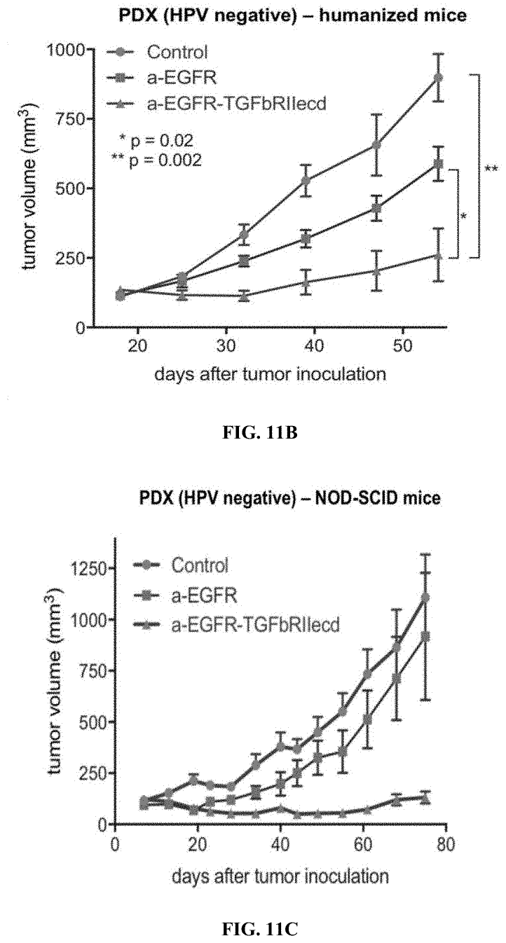

Human tumor xenografts were generated by mammary fat pad implantation of the MDA-MB-231-Luc (D3H2LN) TNBC line in female immune deficient NOG mice (NOD/Shi-scid IL-2rgnull) (FIG. 11A). NOG mice (6-8 week old) were irradiated at 200 cGy and rested for 6-8 h, followed by adoptive transfer of human CD34+ cells (7×104/mouse) from a normal donor (HLA-matched to the TNBC line) (ALLCELLS). Mice were tested for engraftment of human CD3+ T cells in peripheral blood obtained via tail-bleed at 6-7 weeks following injection of CD34+ cells. The cells were stained with anti-huCD3-PE and anti-huCD19-FITC, and analyzed by flow cytometry. Mice demonstrating human CD3+ cell engraftment were injected with MDA-MB-231-Luc cells (2×106 cells in 50% PBS/50% matrigel). At 7d following tumor cell inoculation, mice were randomized and treated for 4 weeks with the following: (i) anti-EGFR-TGFβRII (5 mg/kg i.p. weekly); (ii) anti-EGFR mAb (Cetuximab; 5 mg/kg, i.p, weekly); (iii) anti-TGFβ Ab (1D11; 5 mg/kg i.p. weekly); (iv) Vehicle (Control). Treatment with anti-EGFR-TGFβRII resulted in significantly smaller tumors (257.6±58.5) compared to tumors in mice treated with cetuximab (766.3±64) (p<0.0001) or untreated controls (839.4±77.4)(p<0.0001).

Immune deficient NSG mice (NOD/Shi-scid IL-2rgnull; 6-8 weeks old) were irradiated at 200 cGy and rested for 6-8 h, followed by adoptive transfer of human CD34+ cells (7×104/mouse) from a normal donor (HLA-matched to the D-MUT1 line)(ALLCELLS) (FIG. 11B). Mice were tested for engraftment of human CD3+ T cells in peripheral blood obtained via tail-bleed at 8 weeks following injection of CD34+ cells. The cells were stained with anti-huCD3-PE, and analyzed by flow cytometry. Mice demonstrating human CD3+ cell engraftment were inoculated subcutaneously with a PDX of human HNSCC (moderately-poorly differentiated SCC harvested from the vocal cord). At 18 days following tumor cell inoculation, mice were randomized and treated with the following: (i) anti-EGFR-TGFβRIIecd (5 mg/kg i.p. weekly); (ii) anti-EGFR mAb (Cetuximab; 5 mg/kg, i.p, weekly); (iii) Vehicle (Untreated Control). Treatment with anti-EGFR-TGFβRIIecd resulted in significantly smaller tumors (261.3.1±95.0) compared to tumors in mice treated with cetuximab (588.6.0±61.9) (p=0.03) or untreated controls (898.0±85.4) (p=0.002).

Nude mice were inoculated subcutaneously with a PDX of human HNSCC (SCC harvested from the floor of the mouth) (FIG. 11C). At 21 days following tumor cell inoculation, mice were randomized and treated with the following: (i) anti-EGFR-TGFβRIIecd (5 mg/kg i.p. weekly); (ii) anti-EGFR mAb (Cetuximab; 5 mg/kg, i.p, weekly); (iii) Vehicle (Untreated Control). Treatment with anti-EGFR-TGFβRIIecd resulted in significantly smaller tumors (131.1±29.3) compared to tumors in mice treated with cetuximab (918.4±311.2) (p<0.05) or untreated controls (1107.9±210.0) (p=0.002).

These data demonstrate that tumor-targeted TGFbRII is effective in treating cancer. These data further demonstrate that fusion proteins comprising TGFbRII and a polypeptide that induces or promotes ADCC/FcR-mediated cross-presentation is effective in treating cancer.

### Example 6

### Anti-PD1-TIM3 and Anti-PDL1-TIM3 Fusion Proteins

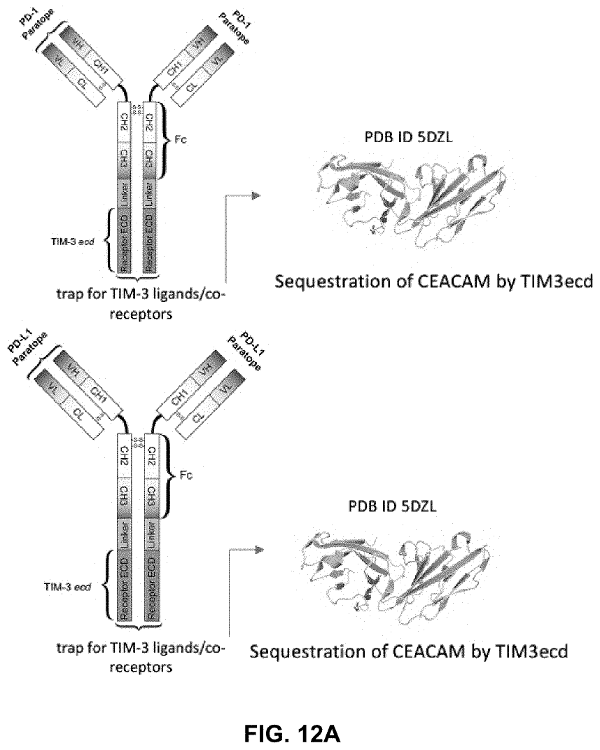

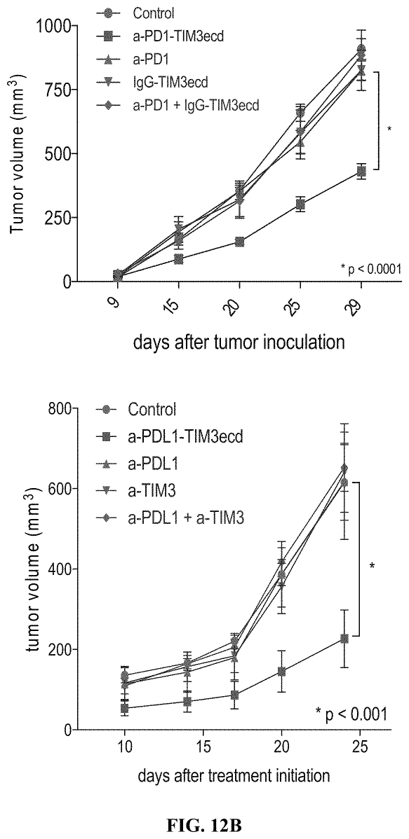

Schematic of anti-PD1-TIM3ecd and anti-PDL1-TIM3ecd (FIG. 12A). Anti-PDL1-TIM3ecd was designed to target both PD-L1 and TIM3 ligands by fusing the C-terminus of the heavy chain of an anti-PDL1 antibody with a ligand binding sequence of the extracellular domain of TIM3 (TIM3ecd) via a flexible linker peptide, (GGGGS)3. Anti-PD1-TIM3ecd was designed to target both PD-1 and TIM3 ligands by fusing the C-terminus of the heavy chain of an anti-PD1 antibody with a ligand binding sequence of the extracellular domain of TIM3 (TIM3ecd) via a flexible linker peptide, (GGGGS)3.

The ability of anti-PD1-TIM3ecd and anti-PDL1-TIM3ecd to elicit antitumor immunity and inhibit the growth of cancers that are refractory to current checkpoint inhibitors, such as triple-negative breast cancer (TNBC) was investigated. Approximately 15-25% of patients with breast cancer have TNBC, an aggressive type that does not respond to hormonal agents or targeted therapy and has an increased risk of metastases. We used human immune reconstituted NSG mice bearing the bioluminescent human MDA-MB-231-luc (D3H2LN) TNBC cell line that expresses elevated PD-L1.

Anti-PD1-TIM3ecd (430.3±29.9) inhibits tumor growth significantly more effectively than untreated control (908.2±40.3), anti-PD-1 (824.0±38.3), IgG-TIM3ecd (825.1±79.0) or the combination of IgG-TIM3ecd and anti-PD1 (884.7±97.4) (p<0.0001) (FIG. 12B).

NSG mice immune reconstituted with tumor-matched HLA A2+ human CD34+ HSC and bearing MDA-MB23 1-Luc human TNBC tumor xenografts were treated (5 mg/kg i.p. every 6 days) with vehicle alone (untreated control) or the following antibodies (either alone or in combination), as indicated: with either vehicle alone (untreated control) or the following antibodies: anti-PD1-TIM3ecd; anti-PD1 (nivolumab); anti-TIM-3 (F38-2E2); or combination of anti-PD1 and anti-TIM-3.

Anti-PDL1-TIM3ecd (226.4±71.4) inhibits tumor growth more effectively than anti-PDL1 (617.5±144.3), anti-TIM-3 (640.9±99.6) or the combination of anti-TIM-3 and anti-PDL1 mAbs (653.0±59.8) (p<0.001) (FIG. 12B).

Treatment of MDA-MB-231-luc-bearing mice with either anti-PDL1, anti-PD1, anti-TIM3, or the combination of anti-PDL1 and anti-TIM-3 mAbs failed to inhibit tumor growth compared to untreated animals. In contrast, treatment with anti-PDL1-TIM3ecd was significantly more effective at inhibiting the progression of MDA-MB-231-luc tumors compared with untreated controls, or animals treated with either anti-PDL1, anti-PD1, anti-TIM3, or the combination of anti-PDL1 and anti-TIM-3 mAbs. NSG mice immune reconstituted with tumor-matched HLA A2+ human CD34+ HSC and bearing MDA-MB231-Luc human TNBC tumor xenografts were treated (5 mg/kg i.p. every 6 days) with vehicle alone (untreated control) or the following antibodies (either alone or in combination), as indicated: with either vehicle alone (untreated control) or the following antibodies: anti-PDL1-TIM3ecd; anti-PDL1 (atezolizumab); anti-TIM-3; anti-PD-1 (pembrolizumab); combination of anti-PDL1 and anti-TIM-3.

Although the invention has been described with reference to the above examples, it will be understood that modifications and variations are encompassed within the spirit and scope of the invention. Accordingly, the invention is limited only by the following claims.

1-232. (canceled)

233. An isolated molecule comprising a targeting moiety and a ligand trap, wherein
(a) the targeting moiety comprises a polypeptide that inhibits SIRPa binding to CD47; and
(b) the ligand trap comprises an amino acid sequence of the extracellular domain of Transforming growth factor-beta receptor (TGFbR) or a ligand binding fragment thereof.

234. The molecule of claim 233, wherein the targeting moiety comprises an antibody, antibody fragment, antigen-binding domain of an immunoglobulin, single chain variable fragment (scFv), Fc-containing polypeptide, or fusion protein.

235. The molecule of claim 233, wherein the targeting moiety specifically binds SIRPa.

236. The molecule of claim 233, wherein the targeting moiety specifically binds CD47.

237. The molecule of claim 236, wherein the targeting moiety comprises an amino acid sequence of the extracellular domain of SIRPa or a ligand-binding fragment thereof.

238. The molecule of claim 237, wherein the targeting moiety comprises the amino acid sequence corresponding to SEQ ID NO: 174 or a ligand-binding fragment thereof.

239. The molecule of claim 233, wherein the ligand trap comprises an amino acid sequence of the extracellular domain of Transforming growth factor-beta receptor II (TGFbRII) or a ligand-binding fragment thereof.

240. The molecule of claim 239, wherein the ligand trap comprises the amino acid sequence corresponding to SEQ ID NO: 177 or a ligand-binding fragment thereof.

241. The molecule of claim 233, wherein the ligand trap is fused to the targeting moiety via a linker.

242. The molecule of claim 241, wherein the targeting moiety is an Fc-containing polypeptide.

243. The molecule of claim 242, wherein the ligand is trap fused to the targeting moiety via a linker attached to the C terminus of the CH3 region of the Fc-containing polypeptide.

244. The molecule of claim 241, wherein the linker is a flexible linker.

245. The molecule of claim 244, wherein the linker is (GGGGS)n and n is between 1 and 10.

246. The molecule of claim 233, wherein the molecule is a fusion protein comprising the amino acid sequence set forth in SEQ ID NO: 550 or 556.

247. The molecule of claim 233, wherein the molecule is a polypeptide comprising the amino acid sequence set forth in SEQ ID NOs: 390 and 22.

248. A method of treating a neoplastic disease or cancer comprising administrating to a subject in need thereof the molecule of claim 233, thereby treating the neoplastic disease or cancer.

249. The method of claim 248, further comprising administering a therapeutic agent.

250. The method of claim 249, wherein the therapeutic agent is an antibody, antibody-drug conjugate, chemotherapeutic agent, or anti-angiogenic agent.

251. The method of claim 250, wherein the anti-angiogenic agent is a polypeptide that binds VEGF, a polypeptide that binds VEGFR, or a VEGFR kinase inhibitor.

252. The method of claim 251, wherein the therapeutic agent is an immune checkpoint inhibitor or vaccine.
